(19) United States
(12) Patent Application Publication
(10) Pub. No.: US 2024/0318177 A1
(43) Pub. Date: Sep. 26, 2024

(54) MUSCLE TARGETING COMPLEXES AND USES THEREOF FOR TREATING DYSTROPHINOPATHIES

(71) Applicant: Dyne Therapeutics, Inc., Waltham, MA (US)

(72) Inventors: Cody A. Desjardins, Waltham, MA (US); Kim Tang, Waltham, MA (US); James McSwiggen, Arvada, CO (US); Romesh R. Subramanian, Framingham, MA (US); Timothy Weeden, Waltham, MA (US); Mohammed T. Qatanani, Waltham, MA (US); Brendan Quinn, Waltham, MA (US); John Najim, Waltham, MA (US)

(73) Assignee: Dyne Therapeutics, Inc., Waltham, MA (US)

(21) Appl. No.: 18/577,378

(22) PCT Filed: Jul. 8, 2022
(86) PCT No.: PCT/US2022/073529

§ 371 Date: Jan. 8, 2024

Related U.S. Application Data
(60) Provisional application No. 63/220,108, filed on Jul. 9, 2021.

(51) Int. Cl. C12N 15/113; A61K 31/7125; A61K 47/68; C07K 16/28

(52) U.S. Cl. CPC C12N 15/113; A61K 31/7125; A61K 47/6807; A61K 47/6849; C07K 16/2881; C07K 2317/55; C07K 2317/77; C12N 2310/3233; C12N 2310/3513; C12N 2320/32; C12N 2320/33

(57) ABSTRACT

Aspects of the disclosure relate to complexes comprising a muscle-targeting agent covalently linked to a molecular payload. In some embodiments, the muscle-targeting agent specifically binds to an internalizing cell surface receptor on muscle cells. In some embodiments, the molecular payload promotes the expression or activity of a functional dystrophin protein. In some embodiments, the molecular payload is an oligonucleotide, such as an antisense oligonucleotide, e.g., an oligonucleotide that causes exon skipping in a mRNA expressed from a mutant DMD allele.

### RELATED APPLICATIONS

This application claims priority under 35 U.S.C. § 119(e) to U.S. Provisional Application Ser. No. 63/220,108, entitled “MUSCLE TARGETING COMPLEXES AND USES THEREOF FOR TREATING DYSTROPHINOPATHIES”, filed on Jul. 9, 2021, the contents of which are incorporated herein by reference in their entirety.

### FIELD OF THE INVENTION

The present application relates to targeting complexes for delivering molecular payloads (e.g., oligonucleotides) to cells and uses thereof, particularly uses relating to treatment of disease.

### REFERENCE TO AN ELECTRONIC SEQUENCE LISTING

The contents of the electronic sequence listing (D082470063WO00-SEQ-COB.xml; Size: 729,857 bytes; and Date of Creation: Jul. 7, 2022) are herein incorporated by reference in their entirety.

### BACKGROUND OF INVENTION

Dystrophinopathies are a group of distinct neuromuscular diseases that result from mutations in the gene encoding dystrophin. Dystrophinopathies include Duchenne muscular dystrophy, Becker muscular dystrophy, and X-linked dilated cardiomyopathy. The DMD gene (“DMD”), which encodes dystrophin, is a large gene, containing 79 exons and about 2.6 million total base pairs. Numerous mutations in DMD, including exonic frameshift, deletion, substitution, and duplicative mutations, are able to diminish the expression of functional dystrophin, leading to dystrophinopathies. Several agents that target exons of human DMD have been approved by the U.S. Food and Drug Administration (FDA), including casimersen, viltolarsen, golodirsen, and eteplirsen.

### SUMMARY OF INVENTION

According to some aspects, the disclosure provides complexes that target muscle cells for purposes of delivering molecular payloads to those cells, as well as molecular payloads that can be used therein. In some embodiments, complexes provided herein are particularly useful for delivering molecular payloads that increase or restore expression or activity of functional dystrophin protein. In some embodiments, complexes comprise oligonucleotide based molecular payloads that promote expression of functional dystrophin protein through an in-frame exon skipping mechanism or suppression of stop codons, such as by facilitating skipping of DMD exon 44. In some embodiments, molecular payloads provided herein are useful for facilitating exon skipping in a DMD sequence, such as skipping of DMD exon 44. Accordingly, in some embodiments, complexes provided herein comprise muscle-targeting agents (e.g., muscle targeting antibodies) that specifically bind to receptors on the surface of muscle cells for purposes of delivering molecular payloads to the muscle cells. In some embodiments, the complexes are taken up into the cells via a receptor mediated internalization, following which the molecular payload may be released to perform a function inside the cells. For example, complexes engineered to deliver oligonucleotides may release the oligonucleotides such that the oligonucleotides can promote expression of functional dystrophin protein (e.g., through an exon skipping mechanism, such as by facilitating skipping of DMD exon 44) in the muscle cells. In some embodiments, the oligonucleotides are released by endosomal cleavage of covalent linkers connecting oligonucleotides and muscle-targeting agents of the complexes. Complexes and molecular payloads provided herein can be used for treating subjects having a mutated DMD gene, such as a mutated DMD gene that is amenable to exon 44 skipping.

According to some aspects, complexes comprising an anti-transferrin receptor 1 (TfR1) antibody covalently linked to an oligonucleotide configured for inducing skipping of exon 44 in a DMD pre-mRNA are provided herein, wherein the oligonucleotide comprises a region of complementarity that is complementary with at least 8 consecutive nucleotides of any one of SEQ ID NOs: 160-195.

In some embodiments, the anti-TfR1 antibody comprises:
(i) a heavy chain complementarity determining region 1 (CDR-H1) of SEQ ID NO: 33, a heavy chain complementarity determining region 2 (CDR-H2) of SEQ ID NO: 34, a heavy chain complementarity determining region 3 (CDR-H3) of SEQ ID NO: 35, a light chain complementarity determining region 1 (CDR-L1) of SEQ ID NO: 36, a light chain complementarity determining region 2 (CDR-L2) of SEQ ID NO: 37, and a light chain complementarity determining region 3 (CDR-L3) of SEQ ID NO: 32;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6;
(iii) a CDR-H1 of SEQ ID NO: 7, a CDR-H2 of SEQ ID NO: 20, a CDR-H3 of SEQ ID NO: 9, a CDR-L1 of SEQ ID NO: 10, a CDR-L2 of SEQ ID NO: 11, and a CDR-L3 of SEQ ID NO: 6;
(iv) a CDR-H1 of SEQ ID NO: 7, a CDR-H2 of SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6;
(v) a CDR-H1 of SEQ ID NO: 51, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50;
(vi) a CDR-H1 of SEQ ID NO: 64, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
(vii) a CDR-H1 of SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50.

In some embodiments, the anti-TfR1 antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a VH comprising an amino acid sequence at least 85% identical to SEQ ID NO: 69; and/or a VL comprising an amino acid sequence at least 85% identical to SEQ ID NO: 70;
(iii) a VH comprising an amino acid sequence at least 85% identical to SEQ ID NO: 71; and/or a VL comprising an amino acid sequence at least 85% identical to SEQ ID NO: 70;
(iv) a VH comprising an amino acid sequence at least 85% identical to SEQ ID NO: 72; and/or a VL comprising an amino acid sequence at least 85% identical to SEQ ID NO: 70;
(v) a VH comprising an amino acid sequence at least 85% identical to SEQ ID NO: 73; and/or a VL comprising an amino acid sequence at least 85% identical to SEQ ID NO: 74;
(vi) a VH comprising an amino acid sequence at least 85% identical to SEQ ID NO: 73; and/or a VL comprising an amino acid sequence at least 85% identical to SEQ ID NO: 75;
(vii) a VH comprising an amino acid sequence at least 85% identical to SEQ ID NO: 76; and/or a VL comprising an amino acid sequence at least 85% identical to SEQ ID NO: 74;
(viii) a VH comprising an amino acid sequence at least 85% identical to SEQ ID NO: 77; and/or a VL comprising an amino acid sequence at least 85% identical to SEQ ID NO: 78;
(ix) a VH comprising an amino acid sequence at least 85% identical to SEQ ID NO: 79; and/or a VL comprising an amino acid sequence at least 85% identical to SEQ ID NO: 80; or
(x) a VH comprising an amino acid sequence at least 85% identical to SEQ ID NO: 77; and/or a VL comprising an amino acid sequence at least 85% identical to SEQ ID NO: 80.

In some embodiments, the anti-TfR1 antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 69 and a VL comprising the amino acid sequence of SEQ ID NO: 70;
(iii) a VH comprising the amino acid sequence of SEQ ID NO: 71 and a VL comprising the amino acid sequence of SEQ ID NO: 70;
(iv) a VH comprising the amino acid sequence of SEQ ID NO: 72 and a VL comprising the amino acid sequence of SEQ ID NO: 70;
(v) a VH comprising the amino acid sequence of SEQ ID NO: 73 and a VL comprising the amino acid sequence of SEQ ID NO: 74;
(vi) a VH comprising the amino acid sequence of SEQ ID NO: 73 and a VL comprising the amino acid sequence of SEQ ID NO: 75;
(vii) a VH comprising the amino acid sequence of SEQ ID NO: 76 and a VL comprising the amino acid sequence of SEQ ID NO: 74;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.

In some embodiments, the anti-TfR1 antibody is a Fab fragment, a Fab′ fragment, a F(ab′)2 fragment, an scFv, an Fv, or a full-length IgG. In some embodiments, the anti-TfR1 antibody is a Fab fragment.

In some embodiments, the anti-TfR1 antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 97; and/or a light chain comprising an amino acid sequence at least 85% identical to SEQ ID NO: 85;
(iii) a heavy chain comprising an amino acid sequence at least 85% identical to SEQ ID NO: 98; and/or a light chain comprising an amino acid sequence at least 85% identical to SEQ ID NO: 85;
(iv) a heavy chain comprising an amino acid sequence at least 85% identical to SEQ ID NO: 99; and/or a light chain comprising an amino acid sequence at least 85% identical to SEQ ID NO: 85;
(v) a heavy chain comprising an amino acid sequence at least 85% identical to SEQ ID NO: 100; and/or a light chain comprising an amino acid sequence at least 85% identical to SEQ ID NO: 89;
(vi) a heavy chain comprising an amino acid sequence at least 85% identical to SEQ ID NO: 100; and/or a light chain comprising an amino acid sequence at least 85% identical to SEQ ID NO: 90;
(vii) a heavy chain comprising an amino acid sequence at least 85% identical to SEQ ID NO: 101; and/or a light chain comprising an amino acid sequence at least 85% identical to SEQ ID NO: 89;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.

In some embodiments, the anti-TfR1 antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 97; and a light chain comprising the amino acid sequence of SEQ ID NO: 85;
(iii) a heavy chain comprising the amino acid sequence of SEQ ID NO: 98; and a light chain comprising the amino acid sequence of SEQ ID NO: 85;
(iv) a heavy chain comprising the amino acid sequence of SEQ ID NO: 99; and a light chain comprising the amino acid sequence of SEQ ID NO: 85;
(v) a heavy chain comprising the amino acid sequence of SEQ ID NO: 100; and a light chain comprising the amino acid sequence of SEQ ID NO: 89;
(vi) a heavy chain comprising the amino acid sequence of SEQ ID NO: 100; and a light chain comprising the amino acid sequence of SEQ ID NO: 90;
(vii) a heavy chain comprising the amino acid sequence of SEQ ID NO: 101; and a light chain comprising the amino acid sequence of SEQ ID NO: 89;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.

In some embodiments, the anti-TfR1 antibody does not specifically bind to the transferrin binding site of the transferrin receptor 1 and/or the anti-TfR1 antibody does not inhibit binding of transferrin to the transferrin receptor 1.

In some embodiments, the oligonucleotide comprises a region of complementarity to at least 4 consecutive nucleotides of a splicing feature of the DMD pre-mRNA.

In some embodiments, the splicing feature is an exonic splicing enhancer (ESE) in exon 44 of the DMD pre-mRNA, optionally wherein the ESE comprises a sequence of any one of SEQ ID NOs: 286-296.

In some embodiments, the splicing feature is a branch point, a splice donor site, or a splice acceptor site, optionally wherein the splicing feature is across the junction of exon 43 and intron 43, in intron 43, across the junction of intron 43 and exon 44, across the junction of exon 44 and intron 44, in intron 44, or across the junction of intron 44 and exon 45 of the DMD pre-mRNA, and further optionally wherein the splicing feature comprises a sequence of any one of SEQ ID NOs: 282-285 and 297-301.

In some embodiments, the oligonucleotide comprises a sequence complementary to any one of SEQ ID NOs: 160-195 or comprises a sequence of any one of SEQ ID NOs: 196-267, wherein each thymine base (T) may independently and optionally be replaced with a uracil base (U), and each U may independently and optionally be replaced with a T.

In some embodiments, the oligonucleotide comprises one or more phosphorodiamidate morpholinos, optionally wherein the oligonucleotide is a phosphorodiamidate morpholino oligomer (PMO).

In some embodiments, the anti-TfR1 antibody is covalently linked to the oligonucleotide via a cleavable linker, optionally wherein the cleavable linker comprises a valine-citrulline sequence.

In some embodiments, the anti-TfR1 antibody is covalently linked to the oligonucleotide via conjugation to a lysine residue or a cysteine residue of the antibody.

According to some aspects, oligonucleotides that target DMD are provided herein, wherein the oligonucleotide comprises a region of complementarity to any one of SEQ ID NOs: 160-195, optionally wherein the region of complementarity comprises at least 15 consecutive nucleosides complementary to any one of SEQ ID NOs: 160-195.

In some embodiments, the oligonucleotide comprises at least 15 consecutive nucleosides of any one of SEQ ID NOs: 196-267, optionally wherein the oligonucleotide comprises a sequence of any one of SEQ ID NOs: 196-267, wherein each thymine base (T) may independently and optionally be replaced with a uracil base (U), and each U may independently and optionally be replaced with a T.

According to some aspects, methods of delivering an oligonucleotide to a cell are provided herein, wherein the method comprises contacting the cell with a complex disclosed herein or with an oligonucleotide disclosed herein.

According to some aspects, methods of promoting the expression or activity of a dystrophin protein in a cell are provided herein, wherein the method comprises contacting the cell with a complex disclosed or with an oligonucleotide disclosed herein in an amount effective for promoting internalization of the oligonucleotide to the cell, optionally wherein the cell is a muscle cell.

In some embodiments, the cell comprises a DMD gene that is amenable to skipping of exon 44.

In some embodiments, the dystrophin protein is a truncated dystrophin protein.

### BRIEF DESCRIPTION OF THE DRAWINGS

FIG. 1 shows data illustrating that conjugates containing anti-TfR1 Fab (3M12 VH4/Vκ3) conjugated to a DMD exon-skipping oligonucleotide resulted in enhanced exon skipping compared to the naked DMD exon skipping oligo in Duchenne muscular dystrophy patient myotubes.

### DETAILED DESCRIPTION OF INVENTION

Aspects of the disclosure relate to a recognition that while certain molecular payloads (e.g., oligonucleotides, peptides, small molecules) can have beneficial effects in muscle cells, it has proven challenging to effectively target such cells. Accordingly, as described herein, the present disclosure provides complexes comprising muscle-targeting agents covalently linked to molecular payloads in order to overcome such challenges. In some embodiments, the complexes are particularly useful for delivering molecular payloads that modulate (e.g., promote) the expression or activity of dystrophin protein (e.g., a truncated dystrophin protein) or DMD (e.g., a mutated DMD allele). In some embodiments, complexes provided herein may comprise oligonucleotides that promote expression and activity of dystrophin protein or DMD, such as by facilitating in-frame exon skipping and/or suppression of premature stop codons. For example, complexes may comprise oligonucleotides that induce skipping of exon(s) of DMD RNA (e.g., pre-mRNA), such as oligonucleotides that induce skipping of exon 44. In some embodiments, synthetic nucleic acid payloads (e.g., DNA or RNA payloads) may be used that express one or more proteins that promote normal expression and activity of dystrophin protein or DMD.

Duchenne muscular dystrophy is an X-linked muscular disorder caused by one or more mutations in the DMD gene located on Xp21. Dystrophin protein typically forms the dystrophin-associated glycoprotein complex (DGC) at the sarcolemma, which links the muscle sarcomeric structure to the extracellular matrix and protects the sarcolemma from contraction-induced injury. In patients with Duchenne muscular dystrophy, the dystrophin protein is generally absent and muscle fibers typically become damaged due to mechanical overextension. Mutations in the DMD gene are associated with two types of muscular dystrophy, Duchenne muscular dystrophy and Becker muscular dystrophy, depending on whether the translational reading frame is lost or maintained. Becker muscular dystrophy is a clinically milder form of Duchenne muscular dystrophy, and is characterized by features similar to Duchenne muscular dystrophy. In some embodiments, exon skipping induced by oligonucleotides (e.g., delivered using complexes provided herein) can be used to restore the reading frame of a mutated DMD allele resulting in production of a truncated dystrophin protein that is sufficiently functional to improve muscle function. In some embodiments, such exon skipping converts a Duchenne muscular dystrophy phenotype into a milder Becker muscular dystrophy phenotype.

Further aspects of the disclosure, including a description of defined terms, are provided below.

### I. Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (A), epsilon (e), gamma (γ) or mu (p) heavy chain. In some embodiments, the heavy chain of an antibody described herein can comprise a human alpha (a), delta (A), epsilon (e), gamma (γ) or mu (p) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Branch point: As used herein, the term “branch point” or “branch site” refers to a nucleic acid sequence motif within an intron of a gene or pre-mRNA that is involved in splicing of pre-mRNA into mRNA (i.e., removing introns from the pre-mRNA), and can be referred to as a splicing feature. A branch point is typically located 18 to 40 nucleotides from the 3′ end of an intron, and contains an adenine but is otherwise relatively unrestricted in sequence. Common sequence motifs for branch points are YNYYRAY, YTRAC, and YNYTRAY, where Y is a pyrimidine, N is any nucleotide, R is any purine, and A is adenine. During splicing, the pre-mRNA is cleaved at the 5′ end of the intron, which then attaches to the branch point region downstream through transesterification bonding between guanines and adenines from the 5′ end and the branch point, respectively, to form a looped lariat structure.

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleosides or two sets of nucleosides. In particular, complementary is a term that characterizes an extent of hydrogen bond pairing that brings about binding between two nucleosides or two sets of nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

DMD: As used herein, the term “DMD” refers to a gene that encodes dystrophin protein, a key component of the dystrophin-glycoprotein complex, which bridges the inner cytoskeleton and the extracellular matrix in muscle cells, particularly muscle fibers. Deletions, duplications, and point mutations in DMD may cause dystrophinopathies, such as Duchenne muscular dystrophy, Becker muscular dystrophy, or cardiomyopathy. Alternative promoter usage and alternative splicing result in numerous distinct transcript variants and protein isoforms for this gene. In some embodiments, a dystrophin gene (DMD or DMD gene) may be a human (Gene ID: 1756), non-human primate (e.g., Gene ID: 465559), or rodent gene (e.g., Gene ID: 13405; Gene ID: 24907). In addition, multiple human transcript variants (e.g., as annotated under GenBank RefSeq Accession Numbers: NM_000109.3, NM_004006.2, NM_004009.3, NM_004010.3 and NM_004011.3) have been characterized that encode different protein isoforms.

DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a dystrophin allele, e.g., exon 8, exon 23, exon 41, exon 44, exon 45, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657-66, the contents of which are incorporated herein by reference in its entirety.

Dystrophinopathy: As used herein, the term “dystrophinopathy” refers to a muscle disease results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.

Exonic splicing enhancer (ESE): As used herein, the term “exonic splicing enhancer” or “ESE” refers to a nucleic acid sequence motif within an exon of a gene, pre-mRNA, or mRNA that directs or enhances splicing of pre-mRNA into mRNA, e.g., as described in Blencowe et al., Trends Biochem Sci 25, 106-10. (2000), incorporated herein by reference. ESEs can be referred to as splicing features. ESEs may direct or enhance splicing, for example, to remove one or more introns and/or one or more exons from a gene transcript. ESE motifs are typically 6-8 nucleobases in length. SR proteins (e.g., proteins encoded by the gene SRSF1, SRSF2, SRSF3, SRSF4, SRSF5, SRSF6, SRSF7, SRSF8, SRSF9, SRSF10, SRSF11, SRSF12, TRA2A or TRA2B) bind to ESEs through their RNA recognition motif region to facilitate splicing. ESE motifs can be identified through a number of methods, including those described in Cartegni et al., Nucleic Acids Research, 2003, Vol. 31, No. 13, 3568-3571, incorporated herein by reference.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding non-human CDR sequences. In one embodiment, humanized anti-TfR1 antibodies and antigen binding portions are provided. Such antibodies may be generated by obtaining murine anti-TfR1 monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad. Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.

Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkages. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Splice acceptor site: As used herein, the term “splice acceptor site” or “splice acceptor” refers to a nucleic acid sequence motif at the 3′ end of an intron or across an intron/exon junction of a gene or pre-mRNA that is involved in splicing of pre-mRNA into mRNA (i.e., removing introns from the pre-mRNA), and can be referred to as a splicing feature. A splice acceptor site includes a terminal AG sequence at the 3′ end of an intron, which is typically preceded (5′-ward) by a region high in pyrimidines (C/U). Upstream from the splice acceptor site is the branch point. Formation of a lariat loop intermediate structure by a transesterification reaction between the branch point and the splice donor site releases a 3′-OH of the 5′ exon, which subsequently reacts with the first nucleotide of the 3′ exon, thereby joining the exons and releasing the intron lariat. The AG sequence at the 3′ end of the intron in the splice acceptor site is known to be critical for proper splicing, as changing one of these nucleotides results in inhibition of splicing. Rarely, alternative splice acceptor sites have an AC at the 3′ end of the intron, instead of the more common AG. A common splice acceptor site motif has a sequence of or similar to [Y-rich region]-NCAGG or YₓNYAGG, in which Y represents a pyrimidine, N represents any nucleotide, and x is a number from 4 to 20. The cut site follows the AG, which represent the 3′-terminal nucleotides of the excised intron.

Splice donor site: As used herein, the term “splice donor site” or “splice donor” refers to a nucleic acid sequence motif at the 5′ end of an intron or across an exon/intron junction of a gene or pre-mRNA that is involved in splicing of pre-mRNA into mRNA (i.e., removing introns from the pre-mRNA), and can be referred to as a splicing feature. A splice donor site includes a terminal GU sequence at the 5′ end of the intron, within a larger and fairly unconstrained sequence. During splicing, the 2′-OH of a nucleotide within the branch point initiates a transesterification reaction via a nucleophilic attack on the 5′ G of the intron within the splice donor site. The G is thereby cleaved from the pre-mRNA and bonds instead to the branch point nucleotide, forming a loop lariat structure. The 3′ nucleotide of the upstream exon subsequently binds the splice acceptor site, joining the exons and excising the intron. A typical splice donor site has a sequence of or similar to GGGURAGU or AGGURNG, in which R represents a purine and N represents any nucleotide. The cut site precedes the first GU (i.e., GG/GURAGU or AG/GURNG), which represent the 5′-terminal nucleotides of the excised intron.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a mutated DMD gene sequence, e.g., a mutation in an exon of a DMD gene sequence. In some embodiments, a subject has a dystrophinopathy, e.g., Duchenne muscular dystrophy. In some embodiments, a subject is a patient that has a mutation of the DMD gene that is amenable to exon 44 skipping.

Transferrin receptor: As used herein, the term, “transferrin receptor” (also known as TFRC, CD71, p90, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to a target sequences, relative to an unmodified oligonucleotide. Examples of structures of 2′-modified nucleosides are provided below:

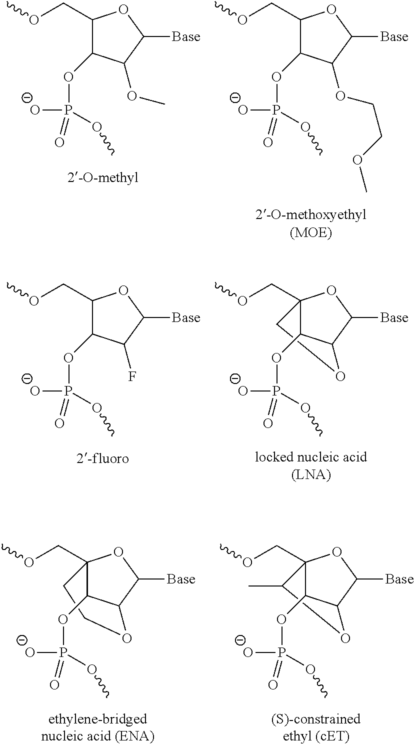

These examples are shown with phosphate groups, but any internucleoside linkages are contemplated between 2′-modified nucleosides.

### II. Complexes

Provided herein are complexes that comprise a targeting agent, e.g. an antibody, covalently linked to a molecular payload. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or (e.g., and) nucleic acid. In some embodiments, the molecular payload present within a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell.

In some embodiments, a complex comprises a muscle-targeting agent, e.g., an anti-transferrin receptor antibody, covalently linked to a molecular payload, e.g., an antisense oligonucleotide that targets DMD to promote exon skipping, e.g., in a transcript encoded from a mutated DMD allele. In some embodiments, the complex targets a DMD pre-mRNA to promote skipping of exon 44 in the DMD pre-mRNA.

### A. Muscle-Targeting Agents

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure, and that any muscle targets (e.g., muscle surface proteins) can be targeted by any type of muscle-targeting agent described herein. For example, the muscle-targeting agent may comprise, or consist of, a small molecule, a nucleic acid (e.g., DNA or RNA), a peptide (e.g., an antibody), a lipid (e.g., a microvesicle), or a sugar moiety (e.g., a polysaccharide). Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or (e.g., and) a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-TfR1 antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or (e.g., and) cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 March, 39(13):78309; the entire contents of each of which are incorporated herein by reference.

a. Anti-Transferrin Receptor (TfR) Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to interchangeably as an, transferrin receptor antibody, an anti-transferrin receptor antibody, or an anti-TfR1 antibody. Antibodies that bind, e.g. specifically bind, to a transferrin receptor may be internalized into the cell, e.g. through receptor-mediated endocytosis, upon binding to a transferrin receptor.

It should be appreciated that anti-TfR1 antibodies may be produced, synthesized, and/or (e.g., and) derivatized using several known methodologies, e.g. library design using phage display. Exemplary methodologies have been characterized in the art and are incorporated by reference (Diez, P. et al. “High-throughput phage-display screening in array format”, Enzyme and microbial technology, 2015, 79, 34-41.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-TfR1 antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052.).

In some embodiments, the anti-TfR1 antibody described herein binds to transferrin receptor with high specificity and affinity. In some embodiments, the anti-TfR1 antibody described herein specifically binds to any extracellular epitope of a transferrin receptor or an epitope that becomes exposed to an antibody. In some embodiments, anti-TfR1 antibodies provided herein bind specifically to transferrin receptor from human, non-human primates, mouse, rat, etc. In some embodiments, anti-TfR1 antibodies provided herein bind to human transferrin receptor. In some embodiments, the anti-TfR1 antibody described herein binds to an amino acid segment of a human or non-human primate transferrin receptor, as provided in SEQ ID NOs: 105-108. In some embodiments, the anti-TfR1 antibody described herein binds to an amino acid segment corresponding to amino acids 90-96 of a human transferrin receptor as set forth in SEQ ID NO: 105, which is not in the apical domain of the transferrin receptor.

In some embodiments, the anti-TfR1 antibodies described herein (e.g., Anti-TfR clone 8 in Table 2 below) bind an epitope in TfR1, wherein the epitope comprises residues in amino acids 214-241 and/or amino acids 354-381 of SEQ ID NO: 105. In some embodiments, the anti-TfR1 antibodies described herein bind an epitope comprising residues in amino acids 214-241 and amino acids 354-381 of SEQ ID NO: 105. In some embodiments, the anti-TfR1 antibodies described herein bind an epitope comprising one or more of residues Y222, T227, K231, H234, T367, S368, S370, T376, and S378 of human TfR1 as set forth in SEQ ID NO: 105. In some embodiments, the anti-TfR1 antibodies described herein bind an epitope comprising residues Y222, T227, K231, H234, T367, S368, S370, T376, and S378 of human TfR1 as set forth in SEQ ID NO: 105.

In some embodiments, the anti-TfR1 antibody described herein (e.g., 3M12 in Table 2 below and its variants) bind an epitope in TfR1, wherein the epitope comprises residues in amino acids 258-291 and/or amino acids 358-381 of SEQ ID NO: 105. In some embodiments, the anti-TfR1 antibodies (e.g., 3M12 in Table 2 below and its variants) described herein bind an epitope comprising residues in amino acids amino acids 258-291 and amino acids 358-381 of SEQ ID NO: 105. In some embodiments, the anti-TfR1 antibodies described herein (e.g., 3M12 in Table 2 below and its variants) bind an epitope comprising one or more of residues K261, S273, Y282, T362, S368, S370, and K371 of human TfR1 as set forth in SEQ ID NO: 105. In some embodiments, the anti-TfR1 antibodies described herein (e.g., 3M12 in Table 2 below and its variants) bind an epitope comprising residues K261, S273, Y282, T362, S368, S370, and K371 of human TfR1 as set forth in SEQ ID NO: 105.

An example human transferrin receptor amino acid sequence, corresponding to NCBI sequence NP_003225.2 (transferrin receptor protein 1 isoform 1, Homo sapiens) is as follows:

(SEQ ID NO: 105)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 106)
MMDQARSAFSNLFGGEPLSYTRFSLARQVDGDNSHVEMKLGVDEEENTD
NNTKPNGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 107)
MMDQARSAFSNLFGGEPLSYTRFSLARQVDGDNSHVEMKLGVDEEENTD
NNTKANGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF.

An example mouse transferrin receptor amino acid sequence, corresponding to NCBI sequence NP_001344227.1 (transferrin receptor protein 1, Mus musculus) is as follows:

(SEQ ID NO: 108)
MMDQARSAFSNLFGGEPLSYTRESLARQVDGDNSHVEMKLAADEEENAD
NNMKASVRKPKRFNGRLCFAAIALVIFFLIGFMSGYLGYCKRVEQKEEC
VKLAETEETDKSETMETEDVPTSSRLYWADLKTLLSEKLNSIEFADTIK
QLSQNTYTPREAGSQKDESLAYYIENQFHEFKFSKVWRDEHYVKIQVKS
SIGQNMVTIVQSNGNLDPVESPEGYVAFSKPTEVSGKLVHANFGTKKDF
EELSYSVNGSLVIVRAGEITFAEKVANAQSFNAIGVLIYMDKNKFPVVE
ADLALFGHAHLGTGDPYTPGFPSFNHTQFPPSQSSGLPNIPVQTISRAA
AEKLFGKMEGSCPARWNIDSSCKLELSQNQNVKLIVKNVLKERRILNIF
GVIKGYEEPDRYVVVGAQRDALGAGVAAKSSVGTGLLLKLAQVFSDMIS
KDGFRPSRSIIFASWTAGDFGAVGATEWLEGYLSSLHLKAFTYINLDKV
VLGTSNFKVSASPLLYTLMGKIMQDVKHPVDGKSLYRDSNWISKVEKLS
FDNAAYPFLAYSGIPAVSFCFCEDADYPYLGTRLDTYEALTQKVPQLNQ
MVRTAAEVAGQLIIKLTHDVELNLDYEMYNSKLLSFMKDLNQFKTDIRD
MGLSLQWLYSARGDYFRATSRLTTDFHNAEKTNRFVMREINDRIMKVEY
HFLSPYVSPRESPFRHIFWGSGSHTLSALVENLKLRQKNITAFNETLFR
NQLALATWTIQGVANALSGDIWNIDNEF

In some embodiments, an anti-TfR1 antibody binds to an amino acid segment of the receptor as follows:

(SEQ ID NO: 109)
FVKIQVKDSAQNSVIIVDKNGRLVYLVENPGGYVAYSKAATVTGKLVHA
NFGTKKDFEDLYTPVNGSIVIVRAGKITFAEKVANAESLNAIGVLIYMD
QTKFPIVNAELSFFGHAHLGTGDPYTPGFPSFNHTQFPPSRSSGLPNIP
VQTISRAAAEKLFGNMEGDCPSDWKTDSTCRMVTSESKNVKLTVSNVLK
E

and does not inhibit the binding interactions between transferrin receptors and transferrin and/or (e.g., and) human hemochromatosis protein (also known as HFE). In some embodiments, the anti-TfR1 antibody described herein does not bind an epitope in SEQ ID NO: 109.

Appropriate methodologies may be used to obtain and/or (e.g., and) produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, the anti-TfR1 antibody of the present disclosure comprises a VL domain and/or (e.g., and) a VH domain of any one of the anti-TfR1 antibodies selected from any one of Tables 2-7, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, agents binding to transferrin receptor, e.g., anti-TfR1 antibodies, are capable of targeting muscle cell and/or (e.g., and) mediate the transportation of an agent across the blood brain barrier. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Antibodies that bind, e.g. specifically bind, to a transferrin receptor may be internalized into the cell, e.g. through receptor-mediated endocytosis, upon binding to a transferrin receptor.

Provided herein, in some aspects, are humanized antibodies that bind to transferrin receptor with high specificity and affinity. In some embodiments, the humanized anti-TfR1 antibody described herein specifically binds to any extracellular epitope of a transferrin receptor or an epitope that becomes exposed to an antibody. In some embodiments, the humanized anti-TfR1 antibodies provided herein bind specifically to transferrin receptor from human, non-human primates, mouse, rat, etc. In some embodiments, the humanized anti-TfR1 antibodies provided herein bind to human transferrin receptor. In some embodiments, the humanized anti-TfR1 antibody described herein binds to an amino acid segment of a human or non-human primate transferrin receptor, as provided in SEQ ID NOs: 105-108. In some embodiments, the humanized anti-TfR1 antibody described herein binds to an amino acid segment corresponding to amino acids 90-96 of a human transferrin receptor as set forth in SEQ ID NO: 105, which is not in the apical domain of the transferrin receptor. In some embodiments, the humanized anti-TfR1 antibodies described herein binds to TfR1 but does not bind to TfR2.

In some embodiments, an anti-TFR1 antibody specifically binds a TfR1 (e.g., a human or non-human primate TfR1) with binding affinity (e.g., as indicated by Kd) of at least about 10⁻⁴ M, 10⁻¹ M, 10⁻⁶ M, 10⁻⁷ M, 10⁻¹ M, 10⁻⁹ M, 10⁻¹⁰ M, 10⁻¹¹ M, 10⁻¹² M, 10⁻¹³ M, or less. In some embodiments, the anti-TfR1 antibodies described herein bind to TfR1 with a KD of sub-nanomolar range. In some embodiments, the anti-TfR1 antibodies described herein selectively bind to transferrin receptor 1 (TfR1) but do not bind to transferrin receptor 2 (TfR2). In some embodiments, the anti-TfR1 antibodies described herein bind to human TfR1 and cyno TfR1 (e.g., with a Kd of 10⁻⁷ M, 10⁻¹ M, 10⁻⁹ M, 10⁻¹⁰ M, 10⁻¹¹ M, 10⁻¹² M, 10⁻¹³ M, or less), but do not bind to a mouse TfR1. The affinity and binding kinetics of the anti-TfR1 antibody can be tested using any suitable method including but not limited to biosensor technology (e.g., OCTET or BIACORE). In some embodiments, binding of any one of the anti-TfR1 antibodies described herein does not complete with or inhibit transferrin binding to the TfR1. In some embodiments, binding of any one of the anti-TfR1 antibodies described herein does not complete with or inhibit HFE-beta-2-microglobulin binding to the TfR 1.

Non-limiting examples of anti-TfR1 antibodies are provided in Table 2.

TABLE 2
Examples of Anti-TfR1 Antibodies

| | No. | | | |
|---|---|---|---|---|
| Ab | system | IMGT | Kabat | Chothia |
| 3-A4 | CDR- | GFNIKDDY (SEQ ID NO: | DDYMY (SEQ ID NO: 7) | GFNIKDD (SEQ ID NO: 12) |
| | H1 | 1) | | |
| | CDR- | IDPENGDT (SEQ ID NO: | WIDPENGDTEYASKFQD | ENG (SEQ ID NO: 13) |
| | H2 | 2) | (SEQ ID NO: 8) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS (SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPENGDT | | |
| | | EYASKFQDKATVTADTSSNTAYLQLSSLTSEDTAVYYCTLWLRRGLDYWGQGTSVTVS | | |
| | | S (SEQ ID NO: 17) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-A4 | CDR- | GFNIKDDY (SEQ ID NO: | DDYMY (SEQ ID NO: 7) | GFNIKDD (SEQ ID NO: 12) |
| N54T* | H1 | 1) | | |
| | CDR- | IDPETGDT (SEQ ID NO: | WIDPETGDTEYASKFQD | ETG (SEQ ID NO: 21) |
| | H2 | 19) | (SEQ ID NO: 20) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS(SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPETGDT | | |
| | | EYASKFQDKATVTADTSSNTAYLQLSSLTSEDTAVYYCTLWLRRGLDYWGQGTSVTVS | | |
| | | S (SEQ ID NO: 22) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-A4 | CDR- | GFNIKDDY (SEQ ID NO: | DDYMY (SEQ ID NO: 7) | GFNIKDD (SEQ ID NO: 12) |
| N54S* | H1 | 1) | | |
| | CDR- | IDPESGDT (SEQ ID NO: | WIDPESGDTEYASKFQD | ESG (SEQ ID NO: 25) |
| | H2 | 23) | (SEQ ID NO: 24) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS (SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPESGDT | | |
| | | EYASKFQDKATVTADTSSNTAYLQLSSLTSEDTAVYYCTLWLRRGLDYWGQGTSVTVS | | |
| | | S (SEQ ID NO: 26) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-M12 | CDR- | GYSITSGYY (SEQ ID | SGYYWN (SEQ ID NO: 33) | GYSITSGY (SEQ ID NO: |
| | H1 | NO: 27) | | 38) |
| | CDR- | ITFDGAN (SEQ ID NO: | YITFDGANNYNPSLKN (SEQ | FDG (SEQ ID NO: 39) |
| | H2 | 28) | ID NO: 34) | |
| | CDR- | TRSSYDYDVLDY (SEQ | SSYDYDVLDY (SEQ ID NO: | SYDYDVLD (SEQ ID NO: |
| | H3 | ID NO: 29) | 35) | 40) |
| | CDR- | QDISNF (SEQ ID NO: 30) | RASQDISNFLN (SEQ ID NO: | SQDISNF (SEQ ID NO: 41) |
| | L1 | | 36) | |
| | CDR- | YTS (SEQ ID NO: 31) | YTSRLHS (SEQ ID NO: 37) | YTS (SEQ ID NO: 31) |
| | L2 | | | |
| | CDR- | QQGHTLPYT (SEQ ID | QQGHTLPYT (SEQ ID NO: 32) | GHTLPY (SEQ ID NO: 42) |
| | L3 | NO: 32) | | |
| | VH | DVQLQESGPGLVKPSQSLSLTCSVTGYSITSGYYWNWIRQFPGNKLEWMGYITFDGAN | | |
| | | NYNPSLKNRISITRDTSKNQFFLKLTSVTTEDTATYYCTRSSYDYDVLDYWGQGTTLTV | | |
| | | SS (SEQ ID NO: 43) | | |
| | VL | DIQMTQTTSSLSASLGDRVTISCRASQDISNFLNWYQQRPDGTVKLLIYYTSRLHSGVPS | | |
| | | RFSGSGSGTDFSLTVSNLEQEDIATYFCQQGHTLPYTFGGGTKLEIK (SEQ ID NO: 44) | | |
| 5-H12 | CDR- | GYSFTDYC (SEQ ID NO: | DYCIN (SEQ ID NO: 51) | GYSFTDY (SEQ ID NO: 56) |
| | H1 | 45) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYCINWVNQRPGQGLEWIGWIYPGSGNTR | | |
| | | YSERFKGKATLTVDTSSNTAYMQLSSLTSEDSAVYFCAREDYYPYHGMDYWGQGTSV | | |
| | | TVSS (SEQ ID NO: 61) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| 5-H12 | CDR- | GYSFTDYY (SEQ ID | DYYIN (SEQ ID NO: 64) | GYSFTDY (SEQ ID NO: 56) |
| C33Y* | H1 | NO: 63) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYYINWVNQRPGQGLEWIGWIYPGSGNTR | | |
| | | YSERFKGKATLTVDTSSNTAYMQLSSLTSEDSAVYFCAREDYYPYHGMDYWGQGTSV | | |
| | | TVSS (SEQ ID NO: 65) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| 5-H12 | CDR- | GYSFTDYD (SEQ ID | DYDIN (SEQ ID NO: 67) | GYSFTDY (SEQ ID NO: 56) |
| C33D* | H1 | NO: 66) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYDINWVNQRPGQGLEWIGWIYPGSGNTRY | | |
| | | SERFKGKATLTVDTSSNTAYMQLSSLTSEDSAVYFCAREDYYPYHGMDYWGQGTSVTV | | |
| | | SS (SEQ ID NO: 68) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| Anti- | CDR- | GYSFTSYW (SEQ ID | SYWIG (SEQ ID NO: 144) | GYSFTSY (SEQ ID NO: |
| TfR | H1 | NO: 138) | | 149) |
| clone 8 | CDR- | IYPGDSDT (SEQ ID NO: | IIYPGDSDTRYSPSFQGQ | GDS (SEQ ID NO: 150) |
| | H2 | 139) | (SEQ ID NO: 145) | |
| | CDR- | ARFPYDSSGYYSFDY | FPYDSSGYYSFDY (SEQ ID | PYDSSGYYSFD (SEQ ID |
| | H3 | (SEQ ID NO: 140) | NO: 146) | NO: 151) |
| | CDR- | QSISSY (SEQ ID NO: | RASQSISSYLN (SEQ ID NO: | SQSISSY (SEQ ID NO: 152) |
| | L1 | 141) | 147) | |
| | CDR- | AAS (SEQ ID NO: 142) | AASSLQS (SEQ ID NO: 148) | AAS (SEQ ID NO: 142) |
| | L2 | | | |
| | CDR- | QQSYSTPLT (SEQ ID | QQSYSTPLT (SEQ ID NO: | SYSTPL (SEQ ID NO: 153) |
| | L3 | NO: 143) | 143) | |

*mutation positions are according to Kabat numbering of the respective VH sequences containing the mutations

In some embodiments, the anti-TfR1 antibody of the present disclosure is a humanized variant of any one of the anti-TfR1 antibodies provided in Table 2. In some embodiments, the anti-TfR1 antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 in any one of the anti-TfR1 antibodies provided in Table 2, and comprises a humanized heavy chain variable region and/or (e.g., and) a humanized light chain variable region.

Examples of amino acid sequences of anti-TfR1 antibodies described herein are provided in Table 3.

TABLE 3
Variable Regions of Anti-TfR1 Antibodies

| Antibody | Variable Region Amino Acid Sequence** |
|---|---|
| 3A4 | V_{H}: |
| VH3 (N54T*)/Vκ4 | EVQLVQSGSELKKPGASVKVSCTASGFNIKDDYMYWVRQPPGKGLEWIGWIDP |
| | ETGDTEYASKFQDRVTVTADTSTNTAYMELSSLRSEDTAVYYCTLWLRRGLD |
| | YWGQGTLVTVSS (SEQ ID NO: 69) |
| | V_{L}: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3A4 | V_{H}: |
| VH3 (N54S*)/Vκ4 | EVQLVQSGSELKKPGASVKVSCTASGFNIKDDYMYWVRQPPGKGLEWIGWIDP |
| | ESGDTEYASKFQDRVTVTADTSTNTAYMELSSLRSEDTAVYYCTLWLRRGLD |
| | YWGQGTLVTVSS (SEQ ID NO: 71) |
| | V_{L}: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3A4 | V_{H}: |
| VH3/Vκ4 | EVQLVQSGSELKKPGASVKVSCTASGFNIKDDYMYWVRQPPGKGLEWIGWIDP |
| | ENGDTEYASKFQDRVTVTADTSTNTAYMELSSLRSEDTAVYYCTLWLRRGLD |
| | YWGQGTLVTVSS (SEQ ID NO: 72) |
| | V_{L}: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGTK |
| | VEIK (SEQ ID NO: 70) |
| 3M12 | V_{H}: |
| VH3/Vκ2 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSS (SEQ ID NO: 73) |
| | V_{L}: |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIK (SEQ |
| | ID NO: 74) |
| 3M12 | V_{H}: |
| VH3/Vκ3 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSS (SEQ ID NO: 73) |
| | V_{L}: |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIK (SEQ |
| | ID NO: 75) |
| 3M12 | V_{H}: |
| VH4/Vκ2 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSS (SEQ ID NO: 76) |
| | V_{L}: |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIK (SEQ |
| | ID NO: 74) |
| 3M12 | V_{H}: |
| VH4/Vκ3 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSS (SEQ ID NO: 76) |
| | V_{L}: |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIK (SEQ |
| | ID NO: 75) |
| 5H12 | V_{H}: |
| VH5 (C33Y*)/Vκ3 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 77) |
| | V_{L}: |
| | DIVLTQSPDSLAVSLGERATINCRASESVDGYDNSFMHWYQQKPGQPPKLLIFR |
| | ASNLESGVPDRFSGSGSRTDFTLTISSLQAEDVAVYYCQQSSEDPWTFGQGTKL |
| | EIK (SEQ ID NO: 78) |
| 5H12 | V_{H}: |
| VH5 (C33D*)/Vκ4 | QVQLVQSGAEVKKPGASVKVSCKASGYSFTDYDINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 79) |
| | V_{L} |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKL |
| | EIK (SEQ ID NO: 80) |
| 5H12 | V_{H}: |
| VH5 (C33Y*)/Vκ4 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSS (SEQ ID NO: 77) |
| | V_{L}: |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKL |
| | EIK (SEQ ID NO: 80) |
| Anti-TfR clone 8 | V_{H}: |
| | QVQLVQSGAEVKKPGESLKISCKGSGYSFTSYWIGWVRQMPGKGLEWMGIIYP |
| | GDSDTRYSPSFQGQVTISADKSISTAYLQWSSLKASDTAMYYCARFPYDSSGYY |
| | SFDYWGQGTLVTVSS (SEQ ID NO: 154) |
| | V_{L}: |
| | DIQMTQSPSSLSASVGDRVTITCRASQSISSYLNWYQQKPGKAPKLLIYAASSLQ |
| | SGVPSRFSGSGSGTDFTLTISSLQPEDFATYYCQQSYSTPLTFGGGTKVEIK (SEQ |
| | ID NO: 155) |

*mutation positions are according to Kabat numbering of the respective VH sequences containing the mutations
**CDRs according to the Kabat numbering system are bolded

In some embodiments, the anti-TfR1 antibody of the present disclosure comprises a VH comprising the CDR-H1, CDR-H2, and CDR-H3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the CDR-H1, CDR-H2, and CDR-H3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the amino acid sequence of SEQ ID NO: 69 and a VL comprising the amino acid sequence of SEQ ID NO: 70.

In some embodiments, the anti-TfR1 antibody of the present disclosure comprises a VH comprising the amino acid sequence of SEQ ID NO: 71 and a VL comprising the amino acid sequence of SEQ ID NO: 70.

In some embodiments, the anti-TfR1 antibody of the present disclosure comprises a VH comprising the amino acid sequence of SEQ ID NO: 72 and a VL comprising the amino acid sequence of SEQ ID NO: 70.

In some embodiments, the anti-TfR1 antibody of the present disclosure comprises a VH comprising the amino acid sequence of SEQ ID NO: 73 and a VL comprising the amino acid sequence of SEQ ID NO: 74.

In some embodiments, the anti-TfR1 antibody of the present disclosure comprises a VH comprising the amino acid sequence of SEQ ID NO: 73 and a VL comprising the amino acid sequence of SEQ ID NO: 75.

In some embodiments, the anti-TfR1 antibody of the present disclosure comprises a VH comprising the amino acid sequence of SEQ ID NO: 76 and a VL comprising the amino acid sequence of SEQ ID NO: 74.

In some embodiments, the anti-TfR1 antibody of the present disclosure comprises a VH comprising the amino acid sequence of SEQ ID NO: 76 and a VL comprising the amino acid sequence of SEQ ID NO: 75.

In some embodiments, the anti-TfR1 antibody of the present disclosure comprises a VH comprising the amino acid sequence of SEQ ID NO: 77 and a VL comprising the amino acid sequence of SEQ ID NO: 78.

In some embodiments, the anti-TfR1 antibody of the present disclosure comprises a VH comprising the amino acid sequence of SEQ ID NO: 79 and a VL comprising the amino acid sequence of SEQ ID NO: 80.

In some embodiments, the anti-TfR1 antibody of the present disclosure comprises a VH comprising the amino acid sequence of SEQ ID NO: 77 and a VL comprising the amino acid sequence of SEQ ID NO: 80.

In some embodiments, the anti-TfR1 antibody of the present disclosure comprises a VH comprising the amino acid sequence of SEQ ID NO: 154 and a VL comprising the amino acid sequence of SEQ ID NO: 155.

In some embodiments, the anti-TfR1 antibody described herein is a full-length IgG, which can include a heavy constant region and a light constant region from a human antibody. In some embodiments, the heavy chain of any of the anti-TfR1 antibodies as described herein may comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 81)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the heavy chain of any of the anti-TfR1 antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcγ receptor binding (Bruhns, P., et al. (2009) and Xu, D. et al. (2000)). The mutant human IgG1 constant region is provided below (mutations bonded and underlined):

(SEQ ID NO: 82)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Other antibody heavy and light chain constant regions are well known in the art, e.g., those provided in the IMGT database (www.imgt.org) or at www.vbase2.org/vbstat.php, both of which are incorporated by reference herein.

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region as set forth in SEQ ID NO: 81. In some embodiments, the anti-TfR1 antibody described herein comprises heavy chain comprising any one of the VH as listed in Table 3 or any variants thereof and a heavy chain constant region as set forth in SEQ ID NO: 82.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of IgG heavy chain and light chain amino acid sequences of the anti-TfR1 antibodies described are provided in Table 4 below.

TABLE 4
Heavy chain and light chain sequences of examples of anti-TfR1 IgGs

| Antibody | IgG Heavy Chain/Light Chain Sequences** |
|---|---|
| 3A4 | Heavy Chain (with wild type human IgG1 constant region) |
| VH3 (N54T*)/Vκ4 | EVQLVQSGSELKKPGASVKVSCTASGFNIKDDYMYWVRQPPGKGLEWIGWIDPE |
| | TGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 84) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with wild type human IgG1 constant region) |
| VH3 (N54S*)/Vκ4 | EVQLVQSGSELKKPGASVKVSCTASGFNIKDDYMYWVRQPPGKGLEWIGWIDPE |
| | SGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 86) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with wild type human IgG1 constant region) |
| VH3/Vκ4 | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 87) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3M12 | Heavy Chain (with wild type human IgG1 constant region) |
| VH3/Vκ2 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with wild type human IgG1 constant region) |
| VH3/Vκ3 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 3M12 | Heavy Chain (with wild type human IgG1 constant region) |
| VH4/Vκ2 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK (SEQ ID NO: 91) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with wild type human IgG1 constant region) |
| VH4/Vκ3 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK (SEQ ID NO: 91) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 5H12 | Heavy Chain (with wild type human IgG1 constant region) |
| VH5 (C33Y*)/Vκ3 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 92) |
| | Light Chain (with kappa light chain constant region) |
| | DIVLTQSPDSLAVSLGERATINCRASESVDGYDNSFMHWYQQKPGQPPKLLIFRAS |
| | NLESGVPDRFSGSGSRTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 93) |
| 5H12 | Heavy Chain (with wild type human IgG1 constant region) |
| VH5 (C33D*)/Vκ4 | QVQLVQSGAEVKKPGASVKVSCKASGYSFTDYDINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 94) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| 5H12 | Heavy Chain (with wild type human IgG1 constant region) |
| VH5 (C33Y*)/Vκ4 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 92) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| Anti-TfR | VH: |
| clone 8 | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 156) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 84, 86, 87, 88, 91, 92, 94, and 156. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 84, 86, 87, 88, 91, 92, 94, and 156. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 84, 86, 87, 88, 91, 92, 94, and 156. Alternatively or in addition (e.g., in addition), the anti-TfR1 antibody described herein comprises a light chain comprising the amino acid sequence of any one of SEQ ID NOs: 85, 89, 90, 93, 95 and 157.

In some embodiments, the anti-TfR1 antibody of the present disclosure comprises a heavy chain comprising the amino acid sequence of SEQ ID NO: 84 and a light chain comprising the amino acid sequence of SEQ ID NO: 85.

In some embodiments, the anti-TfR1 antibody of the present disclosure comprises a heavy chain comprising the amino acid sequence of SEQ ID NO: 86 and a light chain comprising the amino acid sequence of SEQ ID NO: 85.

In some embodiments, the anti-TfR1 antibody of the present disclosure comprises a heavy chain comprising the amino acid sequence of SEQ ID NO: 87 and a light chain comprising the amino acid sequence of SEQ ID NO: 85.

In some embodiments, the anti-TfR1 antibody of the present disclosure comprises a heavy chain comprising the amino acid sequence of SEQ ID NO: 88 and a light chain comprising the amino acid sequence of SEQ ID NO: 89.

In some embodiments, the anti-TfR1 antibody of the present disclosure comprises a heavy chain comprising the amino acid sequence of SEQ ID NO: 88 and a light chain comprising the amino acid sequence of SEQ ID NO: 90.

In some embodiments, the anti-TfR1 antibody of the present disclosure comprises a heavy chain comprising the amino acid sequence of SEQ ID NO: 91 and a light chain comprising the amino acid sequence of SEQ ID NO: 89.

In some embodiments, the anti-TfR1 antibody of the present disclosure comprises a heavy chain comprising the amino acid sequence of SEQ ID NO: 91 and a light chain comprising the amino acid sequence of SEQ ID NO: 90.

In some embodiments, the anti-TfR1 antibody of the present disclosure comprises a heavy chain comprising the amino acid sequence of SEQ ID NO: 92 and a light chain comprising the amino acid sequence of SEQ ID NO: 93.

In some embodiments, the anti-TfR1 antibody of the present disclosure comprises a heavy chain comprising the amino acid sequence of SEQ ID NO: 94 and a light chain comprising the amino acid sequence of SEQ ID NO: 95.

In some embodiments, the anti-TfR1 antibody of the present disclosure comprises a heavy chain comprising the amino acid sequence of SEQ ID NO: 92 and a light chain comprising the amino acid sequence of SEQ ID NO: 95.

In some embodiments, the anti-TfR1 antibody of the present disclosure comprises a heavy chain comprising the amino acid sequence of SEQ ID NO: 156 and a light chain comprising the amino acid sequence of SEQ ID NO: 157.

In some embodiments, the anti-TfR1 antibody is a Fab fragment, Fab′ fragment, or F(ab′)2 fragment of an intact antibody (full-length antibody). Antigen binding fragment of an intact antibody (full-length antibody) can be prepared via routine methods (e.g., recombinantly or by digesting the heavy chain constant region of a full-length IgG using an enzyme such as papain). For example, F(ab′)2 fragments can be produced by pepsin or papain digestion of an antibody molecule, and Fab fragments that can be generated by reducing the disulfide bridges of F(ab′)2 fragments. In some embodiments, a heavy chain constant region in a Fab fragment of the anti-TfR1 antibody described herein comprises the amino acid sequence of:

(SEQ ID NO: 96)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHT

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 96. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 96. In some embodiments, the anti-TfR1 antibody described herein comprises a heavy chain comprising any one of the VH as listed in Table 3 or any variants thereof and a heavy chain constant region as set forth in SEQ ID NO: 96.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of Fab heavy chain and light chain amino acid sequences of the anti-TfR1 antibodies described are provided in Table 5 below.

TABLE 5
Heavy chain and light chain sequences of examples of anti-TfR1 Fabs

| Antibody | Fab Heavy Chain/Light Chain Sequences** |
|---|---|
| 3A4 | Heavy Chain (with partial human IgG1 constant region) |
| VH3 (N54T*)/Vκ4 | EVQLVQSGSELKKPGASVKVSCTASGFNIKDDYMYWVRQPPGKGLEWIGWIDPE |
| | TGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 97) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with partial human IgG1 constant region) |
| VH3 (N54S*)/Vκ4 | EVQLVQSGSELKKPGASVKVSCTASGFNIKDDYMYWVRQPPGKGLEWIGWIDPE |
| | SGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 98) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with partial human IgG1 constant region) |
| VH3/Vκ4 | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 99) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3M12 | Heavy Chain (with partial human IgG1 constant region) |
| VH3/Vκ2 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHT (SEQ ID NO: 100) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with partial human IgG1 constant region) |
| VH3/Vκ3 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHT (SEQ ID NO: 100) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 3M12 | Heavy Chain (with partial human IgG1 constant region) |
| VH4/Vκ2 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHT (SEQ ID NO: 101) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with partial human IgG1 constant region) |
| VH4/Vκ3 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHT (SEQ ID NO: 101) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 5H12 | Heavy Chain (with partial human IgG1 constant region) |
| VH5 (C33Y*)/Vκ3 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 102) |
| | Light Chain (with kappa light chain constant region) |
| | DIVLTQSPDSLAVSLGERATINCRASESVDGYDNSFMHWYQQKPGQPPKLLIFRAS |
| | NLESGVPDRFSGSGSRTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 93) |
| 5H12 | Heavy Chain (with partial human IgG1 constant region) |
| VH5 (C33D*)/Vκ4 | QVQLVQSGAEVKKPGASVKVSCKASGYSFTDYDINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 103) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| 5H12 | Heavy Chain (with partial human IgG1 constant region) |
| VH5 (C33Y*)/Vκ4 | QVQLVQSGAEVKKPGASVKVSCKASGYSFTDYYINWVRQAPGQGLEWMGWIYP |
| | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 102) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| Anti-TfR clone 8 | VH: |
| Version 1 | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCP (SEQ ID NO: 158) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |
| Anti-TfR clone 8 | VH: |
| Version 2 | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 159) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 97-103, 158 and 159. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 97-103, 158 and 159. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 97-103, 158 and 159. Alternatively or in addition (e.g., in addition), the anti-TfR1 antibody described herein comprises a light chain comprising the amino acid sequence of any one of SEQ ID NOs: 85, 89, 90, 93, 95, and 157.

In some embodiments, the anti-TfR1 antibody of the present disclosure comprises a heavy chain comprising the amino acid sequence of SEQ ID NO: 97 and a light chain comprising the amino acid sequence of SEQ ID NO: 85.

In some embodiments, the anti-TfR1 antibody of the present disclosure comprises a heavy chain comprising the amino acid sequence of SEQ ID NO: 98 and a light chain comprising the amino acid sequence of SEQ ID NO: 85.

In some embodiments, the anti-TfR1 antibody of the present disclosure comprises a heavy chain comprising the amino acid sequence of SEQ ID NO: 99 and a light chain comprising the amino acid sequence of SEQ ID NO: 85.

In some embodiments, the anti-TfR1 antibody of the present disclosure comprises a heavy chain comprising the amino acid sequence of SEQ ID NO: 100 and a light chain comprising the amino acid sequence of SEQ ID NO: 89.

In some embodiments, the anti-TfR1 antibody of the present disclosure comprises a heavy chain comprising the amino acid sequence of SEQ ID NO: 100 and a light chain comprising the amino acid sequence of SEQ ID NO: 90.

In some embodiments, the anti-TfR1 antibody of the present disclosure comprises a heavy chain comprising the amino acid sequence of SEQ ID NO: 101 and a light chain comprising the amino acid sequence of SEQ ID NO: 89.

In some embodiments, the anti-TfR1 antibody of the present disclosure comprises a heavy chain comprising the amino acid sequence of SEQ ID NO: 101 and a light chain comprising the amino acid sequence of SEQ ID NO: 90.

In some embodiments, the anti-TfR1 antibody of the present disclosure comprises a heavy chain comprising the amino acid sequence of SEQ ID NO: 102 and a light chain comprising the amino acid sequence of SEQ ID NO: 93.

In some embodiments, the anti-TfR1 antibody of the present disclosure comprises a heavy chain comprising the amino acid sequence of SEQ ID NO: 103 and a light chain comprising the amino acid sequence of SEQ ID NO: 95.

In some embodiments, the anti-TfR1 antibody of the present disclosure comprises a heavy chain comprising the amino acid sequence of SEQ ID NO: 102 and a light chain comprising the amino acid sequence of SEQ ID NO: 95.

In some embodiments, the anti-TfR1 antibody of the present disclosure comprises a heavy chain comprising the amino acid sequence of SEQ ID NO: 158 and a light chain comprising the amino acid sequence of SEQ ID NO: 157.

In some embodiments, the anti-TfR1 antibody of the present disclosure comprises a heavy chain comprising the amino acid sequence of SEQ ID NO: 159 and a light chain comprising the amino acid sequence of SEQ ID NO: 157.

### Other Known Anti-TfR1 Antibodies

Any other appropriate anti-TfR1 antibodies known in the art may be used as the muscle-targeting agent in the complexes disclosed herein. Examples of known anti-TfR1 antibodies, including associated references and binding epitopes, are listed in Table 6. In some embodiments, the anti-TfR1 antibody comprises the complementarity determining regions (CDR-H1, CDR-H2, CDR-H3, CDR-L1, CDR-L2, and CDR-L3) of any of the anti-TfR1 antibodies provided herein, e.g., anti-TfR1 antibodies listed in Table 6.

TABLE 6
List of anti-TfR1 antibody clones, including associated
references and binding epitope information.

| Antibody Clone | | |
|---|---|---|
| Name | Reference(s) | Epitope/Notes |
| OKT9 | U.S. Pat. No. 4,364,934, filed Dec. 4, 1979, | Apical domain of TfR1 |
| | entitled “MONOCLONAL ANTIBODY TO | (residues 305-366 of |
| | A HUMAN EARLY THYMOCYTE | human TfR1 sequence |
| | ANTIGEN AND METHODS FOR | XM_052730.3, available |
| | PREPARING SAME” | in GenBank) |
| | Schneider C. et al. “Structural features of the | |
| | cell surface receptor for transferrin that is | |
| | recognized by the monoclonal antibody | |
| | OKT9.” J Biol Chem. 1982, 257: 14, 8516- | |
| | 8522. | |
| (From JCR) | WO 2015/098989, filed Dec. 24, 2014, | Apical domain (residues |
| Clone M11 | “Novel anti-Transferrin receptor antibody | 230-244 and 326-347 of |
| Clone M23 | that passes through blood-brain barrier” | TfR1) and protease-like |
| Clone M27 | U.S. Pat. No. 9,994,641, filed | domain (residues 461- |
| Clone B84 | Dec. 24, 2014, “Novel anti-Transferrin | 473) |
| | receptor antibody that passes through | |
| | blood-brain barrier” | |
| (From | WO 2016/081643, filed May 26, 2016, | Apical domain and non- |
| Genentech) | entitled “ANTI-TRANSFERRIN | apical regions |
| 7A4, 8A2, 15D2, | RECEPTOR ANTIBODIES AND | |
| 10D11, 7B10, | METHODS OF USE” | |
| 15G11, 16G5, | U.S. Pat. No. 9,708,406, filed | |
| 13C3, 16G4, | May 20, 2014, “Anti-transferrin receptor | |
| 16F6, 7G7, 4C2, | antibodies and methods of use” | |
| 1B12, and 13D4 | | |
| (From Armagen) | Lee et al. “Targeting Rat Anti-Mouse | |
| 8D3 | Transferrin Receptor Monoclonal Antibodies | |
| | through Blood-Brain Barrier in Mouse” | |
| | 2000, J Pharmacol. Exp. Ther., 292: 1048- | |
| | 1052. | |
| | US Patent App. 2010/077498, filed | |
| | Sep. 11, 2008, entitled “COMPOSITIONS AND | |
| | METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute to | |
| | autophagosome formation in response to | |
| | Group A Streptococcus invasion. Cellular | |
| | microbiology. 16: 1806-21. | |
| DF1513 | Ortiz-Zapater E et al. Trafficking of | |
| | the human transferrin receptor in plant cells: | |
| | effects of tyrphostin A23 and brefeldin A. | |
| | Plant J 48: 757-70 (2006). | |
| 1A1B2, 66IG10, | Commercially available anti- | Novus Biologicals |
| MEM-189, | transferrin receptor antibodies. | 8100 Southpark Way, A- |
| JF0956, 29806, | | 8 Littleton CO 80120 |
| 1A1B2, | | |
| TFRC/1818, | | |
| 1E6, 66Ig10, | | |
| TFRC/1059, | | |
| Q1/71, 23D10, | | |
| 13E4, | | |
| TFRC/1149, ER- | | |
| MP21, | | |
| YTA74.4, BU54, | | |
| 2B6, RI7 217 | | |
| (From INSERM) | US Patent App. 2011/0311544A1, | Does not compete with |
| BA120g | filed Jun. 15, 2005, entitled “ANTI-CD71 | OKT9 |
| | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 7, 2004, entitled “TRANSFERRIN | |
| | RECEPTOR ANTIBODIES” | |
| (Salk Institute) | Trowbridge, I. S. et al. “Anti-transferrin | |
| B3/25 | receptor monoclonal antibody and toxin- | |
| T58/30 | antibody conjugates affect growth of | |
| | human tumour cells.” Nature, 1981, | |
| | volume 294, pages 171-173 | |
| R17 217.1.3, | Commercially available anti- | BioXcell |
| 5E9C11, | transferrin receptor antibodies. | 10 Technology Dr., Suite |
| OKT9 (BE0023 | | 2B |
| clone) | | West Lebanon, NH |
| | | 03784-1671 USA |
| BK19.9, B3/25, | Gatter, K. C. et al. “Transferrin receptors | |
| T56/14 and | in human tissues: their distribution and | |
| T58/1 | possible clinical relevance.” J Clin | |
| | Pathol. 1983 May; 36(5): 539-45. | |

Additional Anti-TfR1 antibody SEQ ID NOs

| Anto-TfR1 antibody | | VH/VL | CDR1 | CDR2 | CDR3 |
|---|---|---|---|---|---|
| CDRH1 (SEQ ID NO: 333) | VH1 | 348 | 341 | 342 | 335 |
| CDRH2 (SEQ ID NO: 334) | VH2 | 349 | 341 | 343 | 335 |
| CDRH3 (SEQ ID NO: 335) | VH3 | 350 | 341 | 344 | 335 |
| CDRL1 (SEQ ID NO: 336) | VH4 | 351 | 341 | 343 | 335 |
| CDRL2 (SEQ ID NO: 337) | VL1 | 352 | 336 | 337 | 115 |
| CDRL3 (SEQ ID NO: 338) | VL2 | 353 | 336 | 337 | 115 |
| VH (SEQ ID NO: 339) | VL3 | 354 | 336 | 345 | 338 |
| VL (SEQ ID NO: 340) | VL4 | 355 | 346 | 347 | 338 |

In some embodiments, anti-TfR1 antibodies of the present disclosure include one or more of the CDR-H (e.g., CDR-H1, CDR-H2, and CDR-H3) amino acid sequences from any one of the anti-TfR1 antibodies selected from Table 6. In some embodiments, anti-TfR1 antibodies include the CDR-L1, CDR-L2, and CDR-L3 as provided for any one of the anti-TfR1 antibodies selected from Table 6. In some embodiments, anti-TfR1 antibodies include the CDR-H1, CDR-H2, CDR-H3, CDR-L1, CDR-L2, and CDR-L3 as provided for any one of the anti-TfR1 antibodies selected from Table 6.

In some embodiments, anti-TfR1 antibodies of the disclosure include any antibody that includes a heavy chain variable domain and/or (e.g., and) a light chain variable domain of any anti-TfR1 antibody, such as any one of the anti-TfR1 antibodies selected from Table 6. In some embodiments, anti-TfR1 antibodies of the disclosure include any antibody that includes the heavy chain variable and light chain variable pairs of any anti-TfR1 antibody, such as any one of the anti-TfR1 antibodies selected from Table 6.

Aspects of the disclosure provide anti-TfR1 antibodies having a heavy chain variable (VH) and/or (e.g., and) a light chain variable (VL) domain amino acid sequence homologous to any of those described herein. In some embodiments, the anti-TfR1 antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-TfR1 antibody, such as any one of the anti-TfR1 antibodies selected from Table 6. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR1 antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR1 antibody, such as any one of the anti-TfR1 antibodies selected from Table 6.

An example of a transferrin receptor antibody that may be used in accordance with the present disclosure is described in International Application Publication WO 2016/081643, incorporated herein by reference. The amino acid sequences of this antibody are provided in Table 7.

TABLE 7
Heavy chain and light chain CDRs of an example of a known anti-TfR1 antibody

| Sequence Type | Kabat | Chothia | Contact |
|---|---|---|---|
| CDR-H1 | SYWMH (SEQ ID | GYTFTSY (SEQ ID NO: 116) | TSYWMH (SEQ ID NO: 118) |
| | NO: 110) | | |
| CDR-H2 | EINPTNGRTNYIE | NPTNGR (SEQ ID NO: 117) | WIGEINPTNGRTN (SEQ ID |
| | KFKS (SEQ ID | | NO: 119) |
| | NO: 111) | | |
| CDR-H3 | GTRAYHY (SEQ | GTRAYHY (SEQ ID NO: | ARGTRA (SEQ ID NO: 120) |
| | ID NO: 112) | 112) | |
| CDR-L1 | RASDNLYSNLA | RASDNLYSNLA (SEQ ID | YSNLAWY (SEQ ID NO: 121) |
| | (SEQ ID NO: 113) | NO: 113) | |
| CDR-L2 | DATNLAD (SEQ | DATNLAD (SEQ ID NO: | LLVYDATNLA (SEQ ID NO: |
| | ID NO: 114) | 114) | 122) |
| CDR-L3 | QHFWGTPLT | QHFWGTPLT (SEQ ID NO: | QHFWGTPL (SEQ ID NO: |
| | (SEQ ID NO: 115) | 115) | 123) |

| | |
|---|---|
| Murine VH | QVQLQQPGAELVKPGASVKLSCKASGYTFTSYWMHWVKQRPGQGLEWIGEINP |
| | TNGRTNYIEKFKSKATLTVDKSSSTAYMQLSSLTSEDSAVYYCARGTRAYHYW |
| | GQGTSVTVSS (SEQ ID NO: 124) |
| Murine VL | DIQMTQSPASLSVSVGETVTITCRASDNLYSNLAWYQQKQGKSPQLLVYDATNL |
| | ADGVPSRFSGSGSGTQYSLKINSLQSEDFGTYYCQHFWGTPLTFGAGTKLELK |
| | (SEQ ID NO: 125) |
| Humanized VH | EVQLVQSGAEVKKPGASVKVSCKASGYTFTSYWMHWVRQAPGQRLEWIGEIN |
| | PTNGRTNYIEKFKSRATLTVDKSASTAYMELSSLRSEDTAVYYCARGTRAYHY |
| | WGQGTMVTVSS (SEQ ID NO: 128) |
| Humanized VL | DIQMTQSPSSLSASVGDRVTITCRASDNLYSNLAWYQQKPGKSPKLLVYDATNL |
| | ADGVPSRFSGSGSGTDYTLTISSLQPEDFATYYCQHFWGTPLTFGQGTKVEIK |
| | (SEQ ID NO: 129) |
| HC of chimeric | QVQLQQPGAELVKPGASVKLSCKASGYTFTSYWMHWVKQRPGQGLEWIGEINP |
| full-length IgG1 | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEAL |
| | HNHYTQKSLSLSPGK (SEQ ID NO: 132) |
| LC of chimeric | DIQMTQSPASLSVSVGETVTITCRASDNLYSNLAWYQQKQGKSPQLLVYDATNL |
| full-length IgG1 | ADGVPSRFSGSGSGTQYSLKINSLQSEDFGTYYCQHFWGTPLTFGAGTKLELKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 133) |
| HC of fully human | EVQLVQSGAEVKKPGASVKVSCKASGYTFTSYWMHWVRQAPGQRLEWIGEIN |
| full-length IgG1 | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEA |
| | LHNHYTQKSLSLSPGK (SEQ ID NO: 134) |
| LC of fully human | DIQMTQSPSSLSASVGDRVTITCRASDNLYSNLAWYQQKPGKSPKLLVYDATNL |
| full-length IgG1 | ADGVPSRFSGSGSGTDYTLTISSLQPEDFATYYCQHFWGTPLTFGQGTKVEIKRT |
| | VAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| | (SEQ ID NO: 135) |
| HC of chimeric | QVQLQQPGAELVKPGASVKLSCKASGYTFTSYWMHWVKQRPGQGLEWIGEINP |
| Fab | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCP (SEQ ID NO: 136) |
| HC of fully human | EVQLVQSGAEVKKPGASVKVSCKASGYTFTSYWMHWVRQAPGQRLEWIGEIN |
| Fab | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCP (SEQ ID NO: 137) |

In some embodiments, the anti-TfR1 antibody of the present disclosure comprises a CDR-H1, a CDR-H2, and a CDR-H3 that are the same as the CDR-H1, CDR-H2, and CDR-H3 shown in Table 7. Alternatively or in addition (e.g., in addition), the anti-TfR1 antibody of the present disclosure comprises a CDR-L1, a CDR-L2, and a CDR-L3 that are the same as the CDR-L1, CDR-L2, and CDR-L3 shown in Table 7.

In some embodiments, the anti-TfR1 antibody of the present disclosure comprises a CDR-L3, which contains no more than 3 amino acid variations (e.g., no more than 3, 2, or 1 amino acid variation) as compared with the CDR-L3 as shown in Table 7. In some embodiments, the anti-TfR1 antibody of the present disclosure comprises a CDR-L3 containing one amino acid variation as compared with the CDR-L3 as shown in Table 7. In some embodiments, the anti-TfR1 antibody of the present disclosure comprises a CDR-L3 of QHFAGTPLT (SEQ ID NO: 126) (according to the Kabat and Chothia definition system) or QHFAGTPL (SEQ ID NO: 127) (according to the Contact definition system). In some embodiments, the anti-TfR1 antibody of the present disclosure comprises a CDR-H1, a CDR-H2, a CDR-H3, a CDR-L1 and a CDR-L2 that are the same as the CDR-H1, CDR-H2, and CDR-H3 shown in Table 7, and comprises a CDR-L3 of QHFAGTPLT (SEQ ID NO: 126) (according to the Kabat and Chothia definition system) or QHFAGTPL (SEQ ID NO: 127) (according to the Contact definition system).

In some embodiments, the anti-TfR1 antibody of the present disclosure comprises heavy chain CDRs that collectively are at least 80% (e.g., 80%, 85%, 90%, 95%, or 98%) identical to the heavy chain CDRs as shown in Table 7. Alternatively or in addition (e.g., in addition), the anti-TfR1 antibody of the present disclosure comprises light chain CDRs that collectively are at least 80% (e.g., 80%, 85%, 90%, 95%, or 98%) identical to the light chain CDRs as shown in Table 7.

In some embodiments, the anti-TfR1 antibody of the present disclosure comprises a VH comprising the amino acid sequence of SEQ ID NO: 124. Alternatively or in addition (e.g., in addition), the anti-TfR1 antibody of the present disclosure comprises a VL comprising the amino acid sequence of SEQ ID NO: 125.

In some embodiments, the anti-TfR1 antibody of the present disclosure comprises a VH comprising the amino acid sequence of SEQ ID NO: 128. Alternatively or in addition (e.g., in addition), the anti-TfR1 antibody of the present disclosure comprises a VL comprising the amino acid sequence of SEQ ID NO: 129.

In some embodiments, the anti-TfR1 antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 128. Alternatively or in addition (e.g., in addition), the anti-TfR1 antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 129.

In some embodiments, the anti-TfR1 antibody of the present disclosure is a full-length IgG1 antibody, which can include a heavy constant region and a light constant region from a human antibody. In some embodiments, the heavy chain of any of the anti-TfR1 antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of human IgG1 constant region is given below:

(SEQ ID NO: 81)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQS
GNSQESVTECDSKDSTYSLSSTLTLSKADYEKHKVYACEVTHQGLSSPV
TKSFNRGEC

In some embodiments, the anti-TfR1 antibody described herein is a chimeric antibody that comprises a heavy chain comprising the amino acid sequence of SEQ ID NO: 132. Alternatively or in addition (e.g., in addition), the anti-TfR1 antibody described herein comprises a light chain comprising the amino acid sequence of SEQ ID NO: 133.

In some embodiments, the anti-TfR1 antibody described herein is a fully human antibody that comprises a heavy chain comprising the amino acid sequence of SEQ ID NO: 134. Alternatively or in addition (e.g., in addition), the anti-TfR1 antibody described herein comprises a light chain comprising the amino acid sequence of SEQ ID NO: 135.

In some embodiments, the anti-TfR1 antibody is an antigen binding fragment (Fab) of an intact antibody (full-length antibody). In some embodiments, the anti-TfR1 Fab described herein comprises a heavy chain comprising the amino acid sequence of SEQ ID NO: 136. Alternatively or in addition (e.g., in addition), the anti-TfR1 Fab described herein comprises a light chain comprising the amino acid sequence of SEQ ID NO: 133. In some embodiments, the anti-TfR1 Fab described herein comprises a heavy chain comprising the amino acid sequence of SEQ ID NO: 137. Alternatively or in addition (e.g., in addition), the anti-TfR1 Fab described herein comprises a light chain comprising the amino acid sequence of SEQ ID NO: 135.

The anti-TfR1 antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the anti-TfR1 antibody described herein is an scFv. In some embodiments, the anti-TfR1 antibody described herein is an scFv-Fab (e.g., scFv fused to a portion of a constant region). In some embodiments, the anti-TfR1 antibody described herein is an scFv fused to a constant region (e.g., human IgG1 constant region as set forth in SEQ ID NO: 81).

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of an anti-TfR1 antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-TfR1 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall′Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-TfR1 antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-TfR1 antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 104).

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

b. Other Muscle-Targeting Antibodies

In some embodiments, the muscle-targeting antibody is an antibody that specifically binds hemojuvelin, caveolin-3, Duchenne muscular dystrophy peptide, myosin IIb or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall′Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Cκ or Cλ. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 324) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 324). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for Duchenne muscular dystrophy. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 325) was identified and this muscle-targeting peptide showed improved binding to C2C12 cells relative to the ASSLNIA (SEQ ID NO: 324) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 326) appeared most frequently. Accordingly, in some embodiments, the muscle-targeting agent comprises the amino acid sequence TARGEHKEEELI (SEQ ID NO: 326).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or (e.g., and) derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 327), CSERSMNFC (SEQ ID NO: 328), CPKTRRVPC (SEQ ID NO: 329), WLSEAGPVVTVRALRGTGSW (SEQ ID NO: 330), ASSLNIA (SEQ ID NO: 324), CMQHSMRVC (SEQ ID NO: 331), and DDTRHWG (SEQ ID NO: 332). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147.).

iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Muscle-Targeting Aptamers

A muscle-targeting agent may be an aptamer, e.g. an RNA aptamer, which preferentially targets muscle cells relative to other cell types. In some embodiments, a muscle-targeting aptamer has not been previously characterized or disclosed. These aptamers may be conceived of, produced, synthesized, and/or (e.g., and) derivatized using any of several methodologies, e.g. Systematic Evolution of Ligands by Exponential Enrichment. Exemplary methodologies have been characterized in the art and are incorporated by reference (Yan, A. C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4: 27-40.). In some embodiments, a muscle-targeting aptamer has been previously disclosed (see, e.g. Phillippou, S. et al. “Selection and Identification of Skeletal-Muscle-Targeted RNA Aptamers.” Mol Ther Nucleic Acids. 2018, 10:199-214.; Thiel, W. H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10⁻¹⁵ kDa, about 1-5 Da, about 1-3 kDa, or smaller.

v. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is any muscle targeting agent described herein (e.g., antibodies, nucleic acids, small molecules, peptides, aptamers, lipids, sugar moieties) that target SLC superfamily of transporters. In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or (e.g., and) lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

### B. Molecular Payloads

Some aspects of the disclosure provide molecular payloads, e.g., for modulating a biological outcome, e.g., the transcription of a DNA sequence, the splicing and processing of a RNA sequence, the expression of a protein, or the activity of a protein. In some embodiments, a molecular payload is linked to, or otherwise associated with a muscle-targeting agent. In some embodiments, such molecular payloads are capable of targeting to a muscle cell, e.g., via specifically binding to a nucleic acid or protein in the muscle cell following delivery to the muscle cell by an associated muscle-targeting agent. It should be appreciated that various types of molecular payloads may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, the molecular payload is an oligonucleotide that comprises a strand having a region of complementarity to a mutated DMD allele. Exemplary molecular payloads are described in further detail herein, however, it should be appreciated that the exemplary molecular payloads provided herein are not meant to be limiting.

i. Oligonucleotides

Aspects of the disclosure relate to oligonucleotides configured to modulate (e.g., increase) expression of dystrophin, e.g., from a DMD allele. In some embodiments, oligonucleotides provided herein are configured to alter splicing of DMD pre-mRNA to promote expression of dystrophin protein (e.g., a functional truncated dystrophin protein). In some embodiments, oligonucleotides provided herein are configured to promote skipping of one or more exons in DMD, e.g., in a mutated DMD allele, in order to restore the reading frame. In some embodiments, the oligonucleotides allow for functional dystrophin protein expression (e.g., as described in Watanabe N, Nagata T, Satou Y, et al. NS-065/NCNP-01: an antisense oligonucleotide for potential treatment of exon 53 skipping in Duchenne muscular dystrophy. Mol Ther Nucleic Acids. 2018; 13:442-449). In some embodiments, oligonucleotides provided are configured to promote skipping of exon 44 to produce a shorter but functional version of dystrophin (e.g., containing an in-frame deletion). In some embodiments, oligonucleotides are provided that promote exon 44 skipping (e.g., which may be relevant in a substantial number of patients, including, for example, patients amenable to exon 44 skipping, such as those having deletions in DMD exons 10-43, 11-43, 13-43, 14-43, 15-43, 16-43, 17-43, 19-43, 21-43, 23-43, 24-43, 25-43, 26-43, 27-43, 28-43, 29-43, 30-43, 31-43, 32-43, 33-43, 34-43, 35-43, 36-43, 37-43, 38-43, 39, 40-43, 41-43, 42-43, 43, 45, 45-54, 45-56, or 45-62).

Table 8 provides non-limiting examples of sequences of oligonucleotides that are useful for targeting DMD, e.g., for exon skipping, and for target sequences within DMD. In some embodiments, an oligonucleotide may comprise any antisense sequence provided in Table 8 or a sequence complementary to a target sequence provided in Table 8.

TABLE 8
Oligonucleotide sequences for targeting DMD.

| SEQ | | SEQ | Antisense | SEQ | Antisense | |
|---|---|---|---|---|---|---|
| ID | Target sequence* | ID | Sequence† | ID | Sequence† | Target |
| NO | (5′ to 3′) | NO | (5′ to 3′) | NO | (5′ to 3′) | Site |
| 160 | CAGAUCUGUUGAG | 196 | GCCGCCAUUUCUC | 232 | GCCGCCATTTCTC | Exon 44 |
| | AAAUGGCGGC | | AACAGAUCUG | | AACAGATCTG | |
| 161 | CAGAUCUGUUGAG | 197 | CGCCGCCAUUUCU | 233 | CGCCGCCATTTCT | Exon 44 |
| | AAAUGGCGGCG | | CAACAGAUCUG | | CAACAGATCTG | |
| 162 | CAGAUCUGUUGAG | 198 | ACGCCGCCAUUUC | 234 | ACGCCGCCATTTC | Exon 44 |
| | AAAUGGCGGCGU | | UCAACAGAUCUG | | TCAACAGATCTG | |
| 163 | AGAUCUGUUGAGA | 199 | GCCGCCAUUUCUC | 235 | GCCGCCATTTCTC | Exon 44 |
| | AAUGGCGGC | | AACAGAUCU | | AACAGATCT | |
| 164 | AGAUCUGUUGAGA | 200 | CGCCGCCAUUUCU | 236 | CGCCGCCATTTCT | Exon 44 |
| | AAUGGCGGCG | | CAACAGAUCU | | CAACAGATCT | |
| 165 | AGAUCUGUUGAGA | 201 | ACGCCGCCAUUUC | 237 | ACGCCGCCATTTC | Exon 44 |
| | AAUGGCGGCGU | | UCAACAGAUCU | | TCAACAGATCT | |
| 166 | AGAUCUGUUGAGA | 202 | AACGCCGCCAUUU | 238 | AACGCCGCCATTT | Exon 44 |
| | AAUGGCGGCGUU | | CUCAACAGAUCU | | CTCAACAGATCT | |
| 167 | GAUCUGUUGAGAA | 203 | GCCGCCAUUUCUC | 239 | GCCGCCATTTCTC | Exon 44 |
| | AUGGCGGC | | AACAGAUC | | AACAGATC | |
| 168 | GAUCUGUUGAGAA | 204 | CGCCGCCAUUUCU | 240 | CGCCGCCATTTCT | Exon 44 |
| | AUGGCGGCG | | CAACAGAUC | | CAACAGATC | |
| 169 | GAUCUGUUGAGAA | 205 | ACGCCGCCAUUUC | 241 | ACGCCGCCATTTC | Exon 44 |
| | AUGGCGGCGU | | UCAACAGAUC | | TCAACAGATC | |
| 170 | GAUCUGUUGAGAA | 206 | AACGCCGCCAUUU | 242 | AACGCCGCCATTT | Exon 44 |
| | AUGGCGGCGUU | | CUCAACAGAUC | | CTCAACAGATC | |
| 171 | GAUCUGUUGAGAA | 207 | AAACGCCGCCAUU | 243 | AAACGCCGCCATT | Exon 44 |
| | AUGGCGGCGUUU | | UCUCAACAGAUC | | TCTCAACAGATC | |
| 172 | UCUGUUGAGAAAU | 208 | GAAAACGCCGCCA | 244 | GAAAACGCCGCCA | Exon 44 |
| | GGCGGCGUUUUC | | UUUCUCAACAGA | | TTTCTCAACAGA | |
| 173 | CUGUUGAGAAAUG | 209 | ACGCCGCCAUUUC | 245 | ACGCCGCCATTTC | Exon 44 |
| | GCGGCGU | | UCAACAG | | TCAACAG | |
| 174 | CUGUUGAGAAAUG | 210 | UGAAAACGCCGCC | 246 | TGAAAACGCCGCC | Exon 44 |
| | GCGGCGUUUUCA | | AUUUCUCAACAG | | ATTTCTCAACAG | |
| 175 | UGAGAAAUGGCGG | 211 | AUAAUGAAAACGC | 247 | ATAATGAAAACGC | Exon 44 |
| | CGUUUUCAUUAU | | CGCCAUUUCUCA | | CGCCATTTCTCA | |
| 176 | GAGAAAUGGCGGC | 212 | CAUAAUGAAAACG | 248 | CATAATGAAAACG | Exon 44 |
| | GUUUUCAUUAUG | | CCGCCAUUUCUC | | CCGCCATTTCTC | |
| 177 | AGAAAUGGCGGCG | 213 | UCAUAAUGAAAAC | 249 | TCATAATGAAAAC | Exon 44 |
| | UUUUCAUUAUGA | | GCCGCCAUUUCU | | GCCGCCATTTCT | |
| 178 | AAUCAGUGGCUAA | 214 | GUUCAGCUUCUGU | 250 | GTTCAGCTTCTGT | Exon 44 |
| | CAGAAGCUGAAC | | UAGCCACUGAUU | | TAGCCACTGATT | |
| 179 | AUCAGUGGCUAAC | 215 | GUUCAGCUUCUGU | 251 | GTTCAGCTTCTGT | Exon 44 |
| | AGAAGCUGAAC | | UAGCCACUGAU | | TAGCCACTGAT | |
| 180 | AUCAGUGGCUAAC | 216 | UGUUCAGCUUCUG | 252 | TGTTCAGCTTCTG | Exon 44 |
| | AGAAGCUGAACA | | UUAGCCACUGAU | | TTAGCCACTGAT | |
| 181 | UCAGUGGCUAACA | 217 | GUUCAGCUUCUGU | 253 | GTTCAGCTTCTGT | Exon 44 |
| | GAAGCUGAAC | | UAGCCACUGA | | TAGCCACTGA | |
| 182 | UCAGUGGCUAACA | 218 | UGUUCAGCUUCUG | 254 | TGTTCAGCTTCTG | Exon 44 |
| | GAAGCUGAACA | | UUAGCCACUGA | | TTAGCCACTGA | |
| 183 | UCAGUGGCUAACA | 219 | CUGUUCAGCUUCU | 255 | CTGTTCAGCTTCT | Exon 44 |
| | GAAGCUGAACAG | | GUUAGCCACUGA | | GTTAGCCACTGA | |
| 184 | CAGUGGCUAACAG | 220 | GUUCAGCUUCUGU | 256 | GTTCAGCTTCTGT | Exon 44 |
| | AAGCUGAAC | | UAGCCACUG | | TAGCCACTG | |
| 185 | CAGUGGCUAACAG | 221 | UGUUCAGCUUCUG | 257 | TGTTCAGCTTCTG | Exon 44 |
| | AAGCUGAACA | | UUAGCCACUG | | TTAGCCACTG | |
| 186 | CAGUGGCUAACAG | 222 | CUGUUCAGCUUCU | 258 | CTGTTCAGCTTCT | Exon 44 |
| | AAGCUGAACAG | | GUUAGCCACUG | | GTTAGCCACTG | |
| 187 | CAGUGGCUAACAG | 223 | ACUGUUCAGCUUC | 259 | ACTGTTCAGCTTC | Exon 44 |
| | AAGCUGAACAGU | | UGUUAGCCACUG | | TGTTAGCCACTG | |
| 188 | AGUGGCUAACAGA | 224 | CUGUUCAGCUUCU | 260 | CTGTTCAGCTTCT | Exon 44 |
| | AGCUGAACAG | | GUUAGCCACU | | GTTAGCCACT | |
| 189 | AGUGGCUAACAGA | 225 | ACUGUUCAGCUUC | 261 | ACTGTTCAGCTTC | Exon 44 |
| | AGCUGAACAGU | | UGUUAGCCACU | | TGTTAGCCACT | |
| 190 | AGUGGCUAACAGA | 226 | AACUGUUCAGCUU | 262 | AACTGTTCAGCTT | Exon 44 |
| | AGCUGAACAGUU | | CUGUUAGCCACU | | CTGTTAGCCACT | |
| 191 | GUGGCUAACAGAA | 227 | ACUGUUCAGCUUC | 263 | ACTGTTCAGCTTC | Exon 44 |
| | GCUGAACAGU | | UGUUAGCCAC | | TGTTAGCCAC | |
| 192 | GUGGCUAACAGAA | 228 | AACUGUUCAGCUU | 264 | AACTGTTCAGCTT | Exon 44 |
| | GCUGAACAGUU | | CUGUUAGCCAC | | CTGTTAGCCAC | |
| 193 | AAAGAUCAGGUUC | 229 | CAUCACCCUUCAG | 265 | CATCACCCTTCAG | Intron 44 |
| | UGAAGGGUGAUG | | AACCUGAUCUUU | | AACCTGATCTTT | |
| 194 | AAGAUCAGGUUCU | 230 | CAUCACCCUUCAG | 266 | CATCACCCTTCAG | Intron 44 |
| | GAAGGGUGAUG | | AACCUGAUCUU | | AACCTGATCTT | |
| 195 | AGAUCAGGUUCUG | 231 | CAUCACCCUUCAG | 267 | CATCACCCTTCAG | Intron 44 |
| | AAGGGUGAUG | | AACCUGAUCU | | AACCTGATCT | |

†Each thymine base (T) in any one of the oligonucleotides and/or target sequences provided in Table 8 may independently and optionally be replaced with a uracil base (U), and/or each U may independently and optionally be replaced with a T. Target sequences listed in Table 8 contain U′s, but binding of a DMD-targeting oligonucleotide to RNA and/or DNA is contemplated.

In some embodiments, an oligonucleotide useful for targeting DMD (e.g., for exon skipping) targets a region of a DMD sequence. In some embodiments, an oligonucleotide useful for targeting DMD (e.g., for exon skipping) targets a region of a DMD RNA (e.g., the Dp427m transcript of SEQ ID NO: 130). In some embodiments, an oligonucleotide useful for targeting DMD (e.g., for exon skipping) comprises a region of complementarity to a DMD RNA (e.g., the Dp427m transcript of SEQ ID NO: 130). In some embodiments, an oligonucleotide useful for targeting DMD (e.g., for exon skipping) comprises a region of complementarity to an exon of a DMD RNA (e.g., SEQ ID NO: 131, 273, or 280). In some embodiments, an oligonucleotide useful for targeting DMD (e.g., for exon skipping) comprises a region of complementarity to an intron of a DMD RNA (e.g., SEQ ID NO: 269 or 277). In some embodiments, an oligonucleotide useful for targeting DMD (e.g., for exon skipping) comprises a region of complementarity to a portion of a DMD sequence (e.g., a sequence provided by any one of SEQ ID NOs: 268, 270, 271, 272, 274, 275, 276, 278, 279, 281, and 323). Examples of DMD sequences are provided below. Each of the DMD sequences provided below include thymine nucleotides (T's), but it should be understood that each sequence can represent a DNA sequence or an RNA sequence in which any or all of the T's would be replaced with uracil nucleotides (U's).

Homo sapiens dystrophin (DMD), transcript variant Dp427m, mRNA (NCBI Reference Sequence: NM_004006.2)

(SEQ ID NO: 130)
TCCTGGCATCAGTTACTGTGTTGACTCACTCAGTGTTGGGATCACTCACTTTCCCCCTACAGGACTCAGATCTGGGA
GGCAATTACCTTCGGAGAAAAACGAATAGGAAAAACTGAAGTGTTACTTTTTTTAAAGCTGCTGAAGTTTGTTGGTT
TCTCATTGTTTTTAAGCCTACTGGAGCAATAAAGTTTGAAGAACTTTTACCAGGTTTTTTTTATCGCTGCCTTGATA
TACACTTTTCAAAATGCTTTGGTGGGAAGAAGTAGAGGACTGTTATGAAAGAGAAGATGTTCAAAAGAAAACATTCA
CAAAATGGGTAAATGCACAATTTTCTAAGTTTGGGAAGCAGCATATTGAGAACCTCTTCAGTGACCTACAGGATGGG
AGGCGCCTCCTAGACCTCCTCGAAGGCCTGACAGGGCAAAAACTGCCAAAAGAAAAAGGATCCACAAGAGTTCATGC
CCTGAACAATGTCAACAAGGCACTGCGGGTTTTGCAGAACAATAATGTTGATTTAGTGAATATTGGAAGTACTGACA
TCGTAGATGGAAATCATAAACTGACTCTTGGTTTGATTTGGAATATAATCCTCCACTGGCAGGTCAAAAATGTAATG
AAAAATATCATGGCTGGATTGCAACAAACCAACAGTGAAAAGATTCTCCTGAGCTGGGTCCGACAATCAACTCGTAA
TTATCCACAGGTTAATGTAATCAACTTCACCACCAGCTGGTCTGATGGCCTGGCTTTGAATGCTCTCATCCATAGTC
ATAGGCCAGACCTATTTGACTGGAATAGTGTGGTTTGCCAGCAGTCAGCCACACAACGACTGGAACATGCATTCAAC
ATCGCCAGATATCAATTAGGCATAGAGAAACTACTCGATCCTGAAGATGTTGATACCACCTATCCAGATAAGAAGTC
CATCTTAATGTACATCACATCACTCTTCCAAGTTTTGCCTCAACAAGTGAGCATTGAAGCCATCCAGGAAGTGGAAA
TGTTGCCAAGGCCACCTAAAGTGACTAAAGAAGAACATTTTCAGTTACATCATCAAATGCACTATTCTCAACAGATC
ACGGTCAGTCTAGCACAGGGATATGAGAGAACTTCTTCCCCTAAGCCTCGATTCAAGAGCTATGCCTACACACAGGC
TGCTTATGTCACCACCTCTGACCCTACACGGAGCCCATTTCCTTCACAGCATTTGGAAGCTCCTGAAGACAAGTCAT
TTGGCAGTTCATTGATGGAGAGTGAAGTAAACCTGGACCGTTATCAAACAGCTTTAGAAGAAGTATTATCGTGGCTT
CTTTCTGCTGAGGACACATTGCAAGCACAAGGAGAGATTTCTAATGATGTGGAAGTGGTGAAAGACCAGTTTCATAC
TCATGAGGGGTACATGATGGATTTGACAGCCCATCAGGGCCGGGTTGGTAATATTCTACAATTGGGAAGTAAGCTGA
TTGGAACAGGAAAATTATCAGAAGATGAAGAAACTGAAGTACAAGAGCAGATGAATCTCCTAAATTCAAGATGGGAA
TGCCTCAGGGTAGCTAGCATGGAAAAACAAAGCAATTTACATAGAGTTTTAATGGATCTCCAGAATCAGAAACTGAA
AGAGTTGAATGACTGGCTAACAAAAACAGAAGAAAGAACAAGGAAAATGGAGGAAGAGCCTCTTGGACCTGATCTTG
AAGACCTAAAACGCCAAGTACAACAACATAAGGTGCTTCAAGAAGATCTAGAACAAGAACAAGTCAGGGTCAATTCT
CTCACTCACATGGTGGTGGTAGTTGATGAATCTAGTGGAGATCACGCAACTGCTGCTTTGGAAGAACAACTTAAGGT
ATTGGGAGATCGATGGGCAAACATCTGTAGATGGACAGAAGACCGCTGGGTTCTTTTACAAGACATCCTTCTCAAAT
GGCAACGTCTTACTGAAGAACAGTGCCTTTTTAGTGCATGGCTTTCAGAAAAAGAAGATGCAGTGAACAAGATTCAC
ACAACTGGCTTTAAAGATCAAAATGAAATGTTATCAAGTCTTCAAAAACTGGCCGTTTTAAAAGCGGATCTAGAAAA
GAAAAAGCAATCCATGGGCAAACTGTATTCACTCAAACAAGATCTTCTTTCAACACTGAAGAATAAGTCAGTGACCC
AGAAGACGGAAGCATGGCTGGATAACTTTGCCCGGTGTTGGGATAATTTAGTCCAAAAACTTGAAAAGAGTACAGCA
CAGATTTCACAGGCTGTCACCACCACTCAGCCATCACTAACACAGACAACTGTAATGGAAACAGTAACTACGGTGAC
CACAAGGGAACAGATCCTGGTAAAGCATGCTCAAGAGGAACTTCCACCACCACCTCCCCAAAAGAAGAGGCAGATTA
CTGTGGATTCTGAAATTAGGAAAAGGTTGGATGTTGATATAACTGAACTTCACAGCTGGATTACTCGCTCAGAAGCT
GTGTTGCAGAGTCCTGAATTTGCAATCTTTCGGAAGGAAGGCAACTTCTCAGACTTAAAAGAAAAAGTCAATGCCAT
AGAGCGAGAAAAAGCTGAGAAGTTCAGAAAACTGCAAGATGCCAGCAGATCAGCTCAGGCCCTGGTGGAACAGATGG
TGAATGAGGGTGTTAATGCAGATAGCATCAAACAAGCCTCAGAACAACTGAACAGCCGGTGGATCGAATTCTGCCAG
TTGCTAAGTGAGAGACTTAACTGGCTGGAGTATCAGAACAACATCATCGCTTTCTATAATCAGCTACAACAATTGGA
GCAGATGACAACTACTGCTGAAAACTGGTTGAAAATCCAACCCACCACCCCATCAGAGCCAACAGCAATTAAAAGTC
AGTTAAAAATTTGTAAGGATGAAGTCAACCGGCTATCAGGTCTTCAACCTCAAATTGAACGATTAAAAATTCAAAGC
ATAGCCCTGAAAGAGAAAGGACAAGGACCCATGTTCCTGGATGCAGACTTTGTGGCCTTTACAAATCATTTTAAGCA
AGTCTTTTCTGATGTGCAGGCCAGAGAGAAAGAGCTACAGACAATTTTTGACACTTTGCCACCAATGCGCTATCAGG
AGACCATGAGTGCCATCAGGACATGGGTCCAGCAGTCAGAAACCAAACTCTCCATACCTCAACTTAGTGTCACCGAC
TATGAAATCATGGAGCAGAGACTCGGGGAATTGCAGGCTTTACAAAGTTCTCTGCAAGAGCAACAAAGTGGCCTATA
CTATCTCAGCACCACTGTGAAAGAGATGTCGAAGAAAGCGCCCTCTGAAATTAGCCGGAAATATCAATCAGAATTTG
AAGAAATTGAGGGACGCTGGAAGAAGCTCTCCTCCCAGCTGGTTGAGCATTGTCAAAAGCTAGAGGAGCAAATGAAT
AAACTCCGAAAAATTCAGAATCACATACAAACCCTGAAGAAATGGATGGCTGAAGTTGATGTTTTTCTGAAGGAGGA
ATGGCCTGCCCTTGGGGATTCAGAAATTCTAAAAAAGCAGCTGAAACAGTGCAGACTTTTAGTCAGTGATATTCAGA
CAATTCAGCCCAGTCTAAACAGTGTCAATGAAGGTGGGCAGAAGATAAAGAATGAAGCAGAGCCAGAGTTTGCTTCG
AGACTTGAGACAGAACTCAAAGAACTTAACACTCAGTGGGATCACATGTGCCAACAGGTCTATGCCAGAAAGGAGGC
CTTGAAGGGAGGTTTGGAGAAAACTGTAAGCCTCCAGAAAGATCTATCAGAGATGCACGAATGGATGACACAAGCTG
AAGAAGAGTATCTTGAGAGAGATTTTGAATATAAAACTCCAGATGAATTACAGAAAGCAGTTGAAGAGATGAAGAGA
GCTAAAGAAGAGGCCCAACAAAAAGAAGCGAAAGTGAAACTCCTTACTGAGTCTGTAAATAGTGTCATAGCTCAAGC
TCCACCTGTAGCACAAGAGGCCTTAAAAAAGGAACTTGAAACTCTAACCACCAACTACCAGTGGCTCTGCACTAGGC
TGAATGGGAAATGCAAGACTTTGGAAGAAGTTTGGGCATGTTGGCATGAGTTATTGTCATACTTGGAGAAAGCAAAC
AAGTGGCTAAATGAAGTAGAATTTAAACTTAAAACCACTGAAAACATTCCTGGCGGAGCTGAGGAAATCTCTGAGGT
GCTAGATTCACTTGAAAATTTGATGCGACATTCAGAGGATAACCCAAATCAGATTCGCATATTGGCACAGACCCTAA
CAGATGGCGGAGTCATGGATGAGCTAATCAATGAGGAACTTGAGACATTTAATTCTCGTTGGAGGGAACTACATGAA
GAGGCTGTAAGGAGGCAAAAGTTGCTTGAACAGAGCATCCAGTCTGCCCAGGAGACTGAAAAATCCTTACACTTAAT
CCAGGAGTCCCTCACATTCATTGACAAGCAGTTGGCAGCTTATATTGCAGACAAGGTGGACGCAGCTCAAATGCCTC
AGGAAGCCCAGAAAATCCAATCTGATTTGACAAGTCATGAGATCAGTTTAGAAGAAATGAAGAAACATAATCAGGGG
AAGGAGGCTGCCCAAAGAGTCCTGTCTCAGATTGATGTTGCACAGAAAAAATTACAAGATGTCTCCATGAAGTTTCG
ATTATTCCAGAAACCAGCCAATTTTGAGCAGCGTCTACAAGAAAGTAAGATGATTTTAGATGAAGTGAAGATGCACT
TGCCTGCATTGGAAACAAAGAGTGTGGAACAGGAAGTAGTACAGTCACAGCTAAATCATTGTGTGAACTTGTATAAA
AGTCTGAGTGAAGTGAAGTCTGAAGTGGAAATGGTGATAAAGACTGGACGTCAGATTGTACAGAAAAAGCAGACGGA
AAATCCCAAAGAACTTGATGAAAGAGTAACAGCTTTGAAATTGCATTATAATGAGCTGGGAGCAAAGGTAACAGAAA
GAAAGCAACAGTTGGAGAAATGCTTGAAATTGTCCCGTAAGATGCGAAAGGAAATGAATGTCTTGACAGAATGGCTG
GCAGCTACAGATATGGAATTGACAAAGAGATCAGCAGTTGAAGGAATGCCTAGTAATTTGGATTCTGAAGTTGCCTG
GGGAAAGGCTACTCAAAAAGAGATTGAGAAACAGAAGGTGCACCTGAAGAGTATCACAGAGGTAGGAGAGGCCTTGA
AAACAGTTTTGGGCAAGAAGGAGACGTTGGTGGAAGATAAACTCAGTCTTCTGAATAGTAACTGGATAGCTGTCACC
TCCCGAGCAGAAGAGTGGTTAAATCTTTTGTTGGAATACCAGAAACACATGGAAACTTTTGACCAGAATGTGGACCA
CATCACAAAGTGGATCATTCAGGCTGACACACTTTTGGATGAATCAGAGAAAAAGAAACCCCAGCAAAAAGAAGACG
TGCTTAAGCGTTTAAAGGCAGAACTGAATGACATACGCCCAAAGGTGGACTCTACACGTGACCAAGCAGCAAACTTG
ATGGCAAACCGCGGTGACCACTGCAGGAAATTAGTAGAGCCCCAAATCTCAGAGCTCAACCATCGATTTGCAGCCAT
TTCACACAGAATTAAGACTGGAAAGGCCTCCATTCCTTTGAAGGAATTGGAGCAGTTTAACTCAGATATACAAAAAT
TGCTTGAACCACTGGAGGCTGAAATTCAGCAGGGGGTGAATCTGAAAGAGGAAGACTTCAATAAAGATATGAATGAA
GACAATGAGGGTACTGTAAAAGAATTGTTGCAAAGAGGAGACAACTTACAACAAAGAATCACAGATGAGAGAAAGCG
AGAGGAAATAAAGATAAAACAGCAGCTGTTACAGACAAAACATAATGCTCTCAAGGATTTGAGGTCTCAAAGAAGAA
AAAAGGCTCTAGAAATTTCTCATCAGTGGTATCAGTACAAGAGGCAGGCTGATGATCTCCTGAAATGCTTGGATGAC
ATTGAAAAAAAATTAGCCAGCCTACCTGAGCCCAGAGATGAAAGGAAAATAAAGGAAATTGATCGGGAATTGCAGAA
GAAGAAAGAGGAGCTGAATGCAGTGCGTAGGCAAGCTGAGGGCTTGTCTGAGGATGGGGCCGCAATGGCAGTGGAGC
CAACTCAGATCCAGCTCAGCAAGCGCTGGCGGGAAATTGAGAGCAAATTTGCTCAGTTTCGAAGACTCAACTTTGCA
CAAATTCACACTGTCCGTGAAGAAACGATGATGGTGATGACTGAAGACATGCCTTTGGAAATTTCTTATGTGCCTTC
TACTTATTTGACTGAAATCACTCATGTCTCACAAGCCCTATTAGAAGTGGAACAACTTCTCAATGCTCCTGACCTCT
GTGCTAAGGACTTTGAAGATCTCTTTAAGCAAGAGGAGTCTCTGAAGAATATAAAAGATAGTCTACAACAAAGCTCA
GGTCGGATTGACATTATTCATAGCAAGAAGACAGCAGCATTGCAAAGTGCAACGCCTGTGGAAAGGGTGAAGCTACA
GGAAGCTCTCTCCCAGCTTGATTTCCAATGGGAAAAAGTTAACAAAATGTACAAGGACCGACAAGGGCGATTTGACA
GATCTGTTGAGAAATGGCGGCGTTTTCATTATGATATAAAGATATTTAATCAGTGGCTAACAGAAGCTGAACAGTTT
CTCAGAAAGACACAAATTCCTGAGAATTGGGAACATGCTAAATACAAATGGTATCTTAAGGAACTCCAGGATGGCAT
TGGGCAGCGGCAAACTGTTGTCAGAACATTGAATGCAACTGGGGAAGAAATAATTCAGCAATCCTCAAAAACAGATG
CCAGTATTCTACAGGAAAAATTGGGAAGCCTGAATCTGCGGTGGCAGGAGGTCTGCAAACAGCTGTCAGACAGAAAA
AAGAGGCTAGAAGAACAAAAGAATATCTTGTCAGAATTTCAAAGAGATTTAAATGAATTTGTTTTATGGTTGGAGGA
AGCAGATAACATTGCTAGTATCCCACTTGAACCTGGAAAAGAGCAGCAACTAAAAGAAAAGCTTGAGCAAGTCAAGT
TACTGGTGGAAGAGTTGCCCCTGCGCCAGGGAATTCTCAAACAATTAAATGAAACTGGAGGACCCGTGCTTGTAAGT
GCTCCCATAAGCCCAGAAGAGCAAGATAAACTTGAAAATAAGCTCAAGCAGACAAATCTCCAGTGGATAAAGGTTTC
CAGAGCTTTACCTGAGAAACAAGGAGAAATTGAAGCTCAAATAAAAGACCTTGGGCAGCTTGAAAAAAAGCTTGAAG
ACCTTGAAGAGCAGTTAAATCATCTGCTGCTGTGGTTATCTCCTATTAGGAATCAGTTGGAAATTTATAACCAACCA
AACCAAGAAGGACCATTTGACGTTCAGGAAACTGAAATAGCAGTTCAAGCTAAACAACCGGATGTGGAAGAGATTTT
GTCTAAAGGGCAGCATTTGTACAAGGAAAAACCAGCCACTCAGCCAGTGAAGAGGAAGTTAGAAGATCTGAGCTCTG
AGTGGAAGGCGGTAAACCGTTTACTTCAAGAGCTGAGGGCAAAGCAGCCTGACCTAGCTCCTGGACTGACCACTATT
GGAGCCTCTCCTACTCAGACTGTTACTCTGGTGACACAACCTGTGGTTACTAAGGAAACTGCCATCTCCAAACTAGA
AATGCCATCTTCCTTGATGTTGGAGGTACCTGCTCTGGCAGATTTCAACCGGGCTTGGACAGAACTTACCGACTGGC
TTTCTCTGCTTGATCAAGTTATAAAATCACAGAGGGTGATGGTGGGTGACCTTGAGGATATCAACGAGATGATCATC
AAGCAGAAGGCAACAATGCAGGATTTGGAACAGAGGCGTCCCCAGTTGGAAGAACTCATTACCGCTGCCCAAAATTT
GAAAAACAAGACCAGCAATCAAGAGGCTAGAACAATCATTACGGATCGAATTGAAAGAATTCAGAATCAGTGGGATG
AAGTACAAGAACACCTTCAGAACCGGAGGCAACAGTTGAATGAAATGTTAAAGGATTCAACACAATGGCTGGAAGCT
AAGGAAGAAGCTGAGCAGGTCTTAGGACAGGCCAGAGCCAAGCTTGAGTCATGGAAGGAGGGTCCCTATACAGTAGA
TGCAATCCAAAAGAAAATCACAGAAACCAAGCAGTTGGCCAAAGACCTCCGCCAGTGGCAGACAAATGTAGATGTGG
CAAATGACTTGGCCCTGAAACTTCTCCGGGATTATTCTGCAGATGATACCAGAAAAGTCCACATGATAACAGAGAAT
ATCAATGCCTCTTGGAGAAGCATTCATAAAAGGGTGAGTGAGCGAGAGGCTGCTTTGGAAGAAACTCATAGATTACT
GCAACAGTTCCCCCTGGACCTGGAAAAGTTTCTTGCCTGGCTTACAGAAGCTGAAACAACTGCCAATGTCCTACAGG
ATGCTACCCGTAAGGAAAGGCTCCTAGAAGACTCCAAGGGAGTAAAAGAGCTGATGAAACAATGGCAAGACCTCCAA
GGTGAAATTGAAGCTCACACAGATGTTTATCACAACCTGGATGAAAACAGCCAAAAAATCCTGAGATCCCTGGAAGG
TTCCGATGATGCAGTCCTGTTACAAAGACGTTTGGATAACATGAACTTCAAGTGGAGTGAACTTCGGAAAAAGTCTC
TCAACATTAGGTCCCATTTGGAAGCCAGTTCTGACCAGTGGAAGCGTCTGCACCTTTCTCTGCAGGAACTTCTGGTG
TGGCTACAGCTGAAAGATGATGAATTAAGCCGGCAGGCACCTATTGGAGGCGACTTTCCAGCAGTTCAGAAGCAGAA
CGATGTACATAGGGCCTTCAAGAGGGAATTGAAAACTAAAGAACCTGTAATCATGAGTACTCTTGAGACTGTACGAA
TATTTCTGACAGAGCAGCCTTTGGAAGGACTAGAGAAACTCTACCAGGAGCCCAGAGAGCTGCCTCCTGAGGAGAGA
GCCCAGAATGTCACTCGGCTTCTACGAAAGCAGGCTGAGGAGGTCAATACTGAGTGGGAAAAATTGAACCTGCACTC
CGCTGACTGGCAGAGAAAAATAGATGAGACCCTTGAAAGACTCCAGGAACTTCAAGAGGCCACGGATGAGCTGGACC
TCAAGCTGCGCCAAGCTGAGGTGATCAAGGGATCCTGGCAGCCCGTGGGCGATCTCCTCATTGACTCTCTCCAAGAT
CACCTCGAGAAAGTCAAGGCACTTCGAGGAGAAATTGCGCCTCTGAAAGAGAACGTGAGCCACGTCAATGACCTTGC
TCGCCAGCTTACCACTTTGGGCATTCAGCTCTCACCGTATAACCTCAGCACTCTGGAAGACCTGAACACCAGATGGA
AGCTTCTGCAGGTGGCCGTCGAGGACCGAGTCAGGCAGCTGCATGAAGCCCACAGGGACTTTGGTCCAGCATCTCAG
CACTTTCTTTCCACGTCTGTCCAGGGTCCCTGGGAGAGAGCCATCTCGCCAAACAAAGTGCCCTACTATATCAACCA
CGAGACTCAAACAACTTGCTGGGACCATCCCAAAATGACAGAGCTCTACCAGTCTTTAGCTGACCTGAATAATGTCA
GATTCTCAGCTTATAGGACTGCCATGAAACTCCGAAGACTGCAGAAGGCCCTTTGCTTGGATCTCTTGAGCCTGTCA
GCTGCATGTGATGCCTTGGACCAGCACAACCTCAAGCAAAATGACCAGCCCATGGATATCCTGCAGATTATTAATTG
TTTGACCACTATTTATGACCGCCTGGAGCAAGAGCACAACAATTTGGTCAACGTCCCTCTCTGCGTGGATATGTGTC
TGAACTGGCTGCTGAATGTTTATGATACGGGACGAACAGGGAGGATCCGTGTCCTGTCTTTTAAAACTGGCATCATT
TCCCTGTGTAAAGCACATTTGGAAGACAAGTACAGATACCTTTTCAAGCAAGTGGCAAGTTCAACAGGATTTTGTGA
CCAGCGCAGGCTGGGCCTCCTTCTGCATGATTCTATCCAAATTCCAAGACAGTTGGGTGAAGTTGCATCCTTTGGGG
GCAGTAACATTGAGCCAAGTGTCCGGAGCTGCTTCCAATTTGCTAATAATAAGCCAGAGATCGAAGCGGCCCTCTTC
CTAGACTGGATGAGACTGGAACCCCAGTCCATGGTGTGGCTGCCCGTCCTGCACAGAGTGGCTGCTGCAGAAACTGC
CAAGCATCAGGCCAAATGTAACATCTGCAAAGAGTGTCCAATCATTGGATTCAGGTACAGGAGTCTAAAGCACTTTA
ATTATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAAAG
GTATTTTGCGAAGCATCCCCGAATGGGCTACCTGCCAGTGCAGACTGTCTTAGAGGGGGACAACATGGAAACTCCCG
TTACTCTGATCAACTTCTGGCCAGTAGATTCTGCGCCTGCCTCGTCCCCTCAGCTTTCACACGATGATACTCATTCA
CGCATTGAACATTATGCTAGCAGGCTAGCAGAAATGGAAAACAGCAATGGATCTTATCTAAATGATAGCATCTCTCC
TAATGAGAGCATAGATGATGAACATTTGTTAATCCAGCATTACTGCCAAAGTTTGAACCAGGACTCCCCCCTGAGCC
AGCCTCGTAGTCCTGCCCAGATCTTGATTTCCTTAGAGAGTGAGGAAAGAGGGGAGCTAGAGAGAATCCTAGCAGAT
CTTGAGGAAGAAAACAGGAATCTGCAAGCAGAATATGACCGTCTAAAGCAGCAGCACGAACATAAAGGCCTGTCCCC
ACTGCCGTCCCCTCCTGAAATGATGCCCACCTCTCCCCAGAGTCCCCGGGATGCTGAGCTCATTGCTGAGGCCAAGC
TACTGCGTCAACACAAAGGCCGCCTGGAAGCCAGGATGCAAATCCTGGAAGACCACAATAAACAGCTGGAGTCACAG
TTACACAGGCTAAGGCAGCTGCTGGAGCAACCCCAGGCAGAGGCCAAAGTGAATGGCACAACGGTGTCCTCTCCTTC
TACCTCTCTACAGAGGTCCGACAGCAGTCAGCCTATGCTGCTCCGAGTGGTTGGCAGTCAAACTTCGGACTCCATGG
GTGAGGAAGATCTTCTCAGTCCTCCCCAGGACACAAGCACAGGGTTAGAGGAGGTGATGGAGCAACTCAACAACTCC
TTCCCTAGTTCAAGAGGAAGAAATACCCCTGGAAAGCCAATGAGAGAGGACACAATGTAGGAAGTCTTTTCCACATG
GCAGATGATTTGGGCAGAGCGATGGAGTCCTTAGTATCAGTCATGACAGATGAAGAAGGAGCAGAATAAATGTTTTA
CAACTCCTGATTCCCGCATGGTTTTTATAATATTCATACAACAAAGAGGATTAGACAGTAAGAGTTTACAAGAAATA
AATCTATATTTTTGTGAAGGGTAGTGGTATTATACTGTAGATTTCAGTAGTTTCTAAGTCTGTTATTGTTTTGTTAA
CAATGGCAGGTTTTACACGTCTATGCAATTGTACAAAAAAGTTATAAGAAAACTACATGTAAAATCTTGATAGCTAA
ATAACTTGCCATTTCTTTATATGGAACGCATTTTGGGTTGTTTAAAAATTTATAACAGTTATAAAGAAAGATTGTAA
ACTAAAGTGTGCTTTATAAAAAAAAGTTGTTTATAAAAACCCCTAAAAACAAAACAAACACACACACACACACATAC
ACACACACACACAAAACTTTGAGGCAGCGCATTGTTTTGCATCCTTTTGGCGTGATATCCATATGAAATTCATGGCT
TTTTCTTTTTTTGCATATTAAAGATAAGACTTCCTCTACCACCACACCAAATGACTACTACACACTGCTCATTTGAG
AACTGTCAGCTGAGTGGGGCAGGCTTGAGTTTTCATTTCATATATCTATATGTCTATAAGTATATAAATACTATAGT
TATATAGATAAAGAGATACGAATTTCTATAGACTGACTTTTTCCATTTTTTAAATGTTCATGTCACATCCTAATAGA
AAGAAATTACTTCTAGTCAGTCATCCAGGCTTACCTGCTTGGTCTAGAATGGATTTTTCCCGGAGCCGGAAGCCAGG
AGGAAACTACACCACACTAAAACATTGTCTACAGCTCCAGATGTTTCTCATTTTAAACAACTTTCCACTGACAACGA
AAGTAAAGTAAAGTATTGGATTTTTTTAAAGGGAACATGTGAATGAATACACAGGACTTATTATATCAGAGTGAGTA
ATCGGTTGGTTGGTTGATTGATTGATTGATTGATACATTCAGCTTCCTGCTGCTAGCAATGCCACGATTTAGATTTA
ATGATGCTTCAGTGGAAATCAATCAGAAGGTATTCTGACCTTGTGAACATCAGAAGGTATTTTTTAACTCCCAAGCA
GTAGCAGGACGATGATAGGGCTGGAGGGCTATGGATTCCCAGCCCATCCCTGTGAAGGAGTAGGCCACTCTTTAAGT
GAAGGATTGGATGATTGTTCATAATACATAAAGTTCTCTGTAATTACAACTAAATTATTATGCCCTCTTCTCACAGT
CAAAAGGAACTGGGTGGTTTGGTTTTTGTTGCTTTTTTAGATTTATTGTCCCATGTGGGATGAGTTTTTAAATGCCA
CAAGACATAATTTAAAATAAATAAACTTTGGGAAAAGGTGTAAAACAGTAGCCCCATCACATTTGTGATACTGACAG
GTATCAACCCAGAAGCCCATGAACTGTGTTTCCATCCTTTGCATTTCTCTGCGAGTAGTTCCACACAGGTTTGTAAG
TAAGTAAGAAAGAAGGCAAATTGATTCAAATGTTACAAAAAAACCCTTCTTGGTGGATTAGACAGGTTAAATATATA
AACAAACAAACAAAAATTGCTCAAAAAAGAGGAGAAAAGCTCAAGAGGAAAAGCTAAGGACTGGTAGGAAAAAGCTT
TACTCTTTCATGCCATTTTATTTCTTTTTGATTTTTAAATCATTCATTCAATAGATACCACCGTGTGACCTATAATT
TTGCAAATCTGTTACCTCTGACATCAAGTGTAATTAGCTTTTGGAGAGTGGGCTGACATCAAGTGTAATTAGCTTTT
GGAGAGTGGGTTTTGTCCATTATTAATAATTAATTAATTAACATCAAACACGGCTTCTCATGCTATTTCTACCTCAC
TTTGGTTTTGGGGTGTTCCTGATAATTGTGCACACCTGAGTTCACAGCTTCACCACTTGTCCATTGCGTTATTTTCT
TTTTCCTTTATAATTCTTTCTTTTTCCTTCATAATTTTCAAAAGAAAACCCAAAGCTCTAAGGTAACAAATTACCAA
ATTACATGAAGATTTGGTTTTTGTCTTGCATTTTTTTCCTTTATGTGACGCTGGACCTTTTCTTTACCCAAGGATTT
TTAAAACTCAGATTTAAAACAAGGGGTTACTTTACATCCTACTAAGAAGTTTAAGTAAGTAAGTTTCATTCTAAAAT
CAGAGGTAAATAGAGTGCATAAATAATTTTGTTTTAATCTTTTTGTTTTTCTTTTAGACACATTAGCTCTGGAGTGA
GTCTGTCATAATATTTGAACAAAAATTGAGAGCTTTATTGCTGCATTTTAAGCATAATTAATTTGGACATTATTTCG
TGTTGTGTTCTTTATAACCACCAAGTATTAAACTGTAAATCATAATGTAACTGAAGCATAAACATCACATGGCATGT
TTTGTCATTGTTTTCAGGTACTGAGTTCTTACTTGAGTATCATAATATATTGTGTTTTAACACCAACACTGTAACAT
TTACGAATTATTTTTTTAAACTTCAGTTTTACTGCATTTTCACAACATATCAGACTTCACCAAATATATGCCTTACT
ATTGTATTATAGTACTGCTTTACTGTGTATCTCAATAAAGCACGCAGTTATGTTAC

Homo sapiens dystrophin (DMD), transcript variant Dp427m, exon 43 (nucleotide positions 6362-6534 of NCBI Reference Sequence: NM_004006.2; nucleotide positions 1056909-1057081 of NCBI Reference Sequence: NG_012232.1)

(SEQ ID NO: 131)
AATATAAAAGATAGTCTACAACAAAGCTCAGGTCGGATTGACATTATTC
ATAGCAAGAAGACAGCAGCATTGCAAAGTGCAACGCCTGTGGAAAGGGT
GAAGCTACAGGAAGCTCTCTCCCAGCTTGATTTCCAATGGGAAAAAGTT
AACAAAATGTACAAGGACCGACAAGG

Homo sapiens dystrophin (DMD) exon 43/intron 43 junction (nucleotide positions 1057052-1057111 of NCBI Reference Sequence: NG_012232.1)

(SEQ ID NO: 268)
AGTTAACAAAATGTACAAGGACCGACAAGGGTAGGTAACACATATATTT
TTCTTGATACT

Homo sapiens dystrophin (DMD), intron 43 (nucleotide positions 1057082-1127546 of NCBI Reference Sequence: NG_012232.1)

(SEQ ID NO: 269)
GTAGGTAACACATATATTTTTCTTGATACTTGCAGAAATGATTTGTTTTCAGGGAACTGTAGAATTTATTTCAGTAC
CCTCCATGGAAAAAAGACAGGGAAAAAGAAGTATCACTCTCATTGAAAAATGGTAAGTAAAATGAGAACAAATAATG
ATGAACAATTCAGACAAATGAGCTGAACTGTAGAAATTATTCAGTATTAAATTTAGCGTTATGTTGAAGCATTATAT
CTTCTAAAATTTCAGGTAATCGTAAATCAGATTGGGGCATTGAGGTGCTATAAACAGTTGTTCGATATAACATATTT
ATCTTTAGTCAAATCTGTATCTACAAATTTATAGGAGTTTGATACTTAGATTAACATGTTTAAAATACTACCTATCA
ATAAAAAAATTGATTTTCATTTATTTATATTTTGGAGGGTGACACTTTACACTTTGCATTACCCTCCCCCACATTTT
TTTTTGCCATGGGGAATTTGTTAAGTTGATTTAAAATCATGTTTCTTTTTATCTTAATTGAAATATTTAGCTGTTGA
CTTAATTGGGGGGTGGCAGAAATTCAATTACATTATTTAGCTTCACATCACAAGAAGTGGATTACTCTTTCTTTTAC
ATTTTTAGATTAAAAGTAATAAAGTTCTCTAACAGCTTAAAGTGATTCATTTCAGAAGTCTTTATCCTCAAGGTAGG
CTTCTTAATTATTCTCAGAGGAAATCAAATCATTCGTCTAATAATTATAGGGAACCTCCATGGTATCTCTTACATCT
GTCCATTTCTAACCATCTTTACTCCTATTTTAGTTCTTGGTTTACTACATCAGGCACCTAAAGAGAGATTTCTAGCA
TTAATCTCAGTTGTCTCAAGTGATTATCTCCACTTTGTTCTCAACGATCTTTCAGTGGCCGTCATTTTTCTAAAGTG
TAAGAGCTTTGGTACTATAATCTCATTCCCTTGAGAATGAAACCCTGACTCCTTAATTGTGCAAAATTATCTGGTCA
GCCTCACTGCTTATTTCTTCGTGCCTGCCCTATAGCAAGCCCCTCCAATTATGTTCTAGCGATATTAAATCAATTTG
CAGATGCCCAAGTGTTTTGTACTCCCTCTGACCTGCCTGTTCTTCAGGTCTCACTTCAGATGTTGTCTTTTTCTAGA
AATCCTAAAACTATTAGTCGCTCCTAACATCTGGAAACTTGGATATTGTTTCATCATTTAATGTAATTTAATATCAA
TAAATCATTTATGCATTTGTAAATTTTCCAGACTTGGTGGCTACCGTGTAGAATATATGATCTGATGTGCCTGCTCC
TAAAACCTAACACATAGTAATCAATTTGTTTACTTTTCTGCCTCCCACACAAGCTCCTTATAGAAAAGGGCTATGTT
TTATTTGTGTTATATGCTCAGGACTGATATAGTGCTGAGCACAAAGTGGGTGTTCAATGTGTCTGGACTCAAGCCTC
CTCTCCGAAACCTGAGAATATTTCCCCTTTGGATAGTCTACCAGATTTATCTCACTGCACCTTCCAGGGCAGGTGCG
GTGGCTCATGTCTGTAATCCCAGCACTTTGGGAGTCCAAGGCGGGCACATCATTTGAGGTCAGGAGTCCGAGACCAG
CCTGGCCAACATGGTGAAACCTTGTCTCCATTAAAAATACAAAAATGACCTGTGCGTGGTGGCATGCGCTTGTAGTC
CCAGCTACTCGGGAGGCTGAGGCAGGAGAATCCCTTGAACCCAGGAGGCAGAGGCTGCAGTGAGCCCAAATCCACCA
CTGCATTCCAGCCTGGGTGACAGAGCAAGGCTCCGTCTCAAACAAAACAAAACAAAACAAAAAAACAGATTTATCTC
AACTTCCTTGAAAATAGCTGGTCCCACAGTATCCAAAGCCAGGCTTGATAAAGGCAAAGAAACTCAGCCACTCTGTG
TATATGCTACAAATACAAACTTAAACTTATAAAATGGTGGCTCATGCCTGTAATCCCTGTGCTTTGGGAGGCTGAGG
CAGGAGGGTCACTTGAGCCCAGGAATTCAAGGCTGCCACGAGCCATGATTGTGCCACTGTACTACAGCCTGAGTAAC
AGACTCTCTCTAAAATAAATTAACAAAAATATTTGAATAAACTTATATCGCCACCATGGTGTACTTTCTCCCAGCAT
TTCACATTGAGACATTATGAGGCTCTGCGGGCTACTCCAATGATGTCAGTTTCATTACCATCTTTTGGGATGTTCAT
TCCACTCTTGGGAAGTTCTTCCCCCTCTGGTTTGTCTTCTTATAGTCAGTGTTTTCTCCAAGCATTCTAGGCCACCC
ACACCATCCCCTAAGGACCGTTGAGGTTTCTTGGATAGCCAGTGATTACTGTGCTTCTATATACACCTACCAGTTTT
GCACTAAAGTAGAAATAACGCTTTCTGCTTTTTGAGGTTTCTCCTCCGCTTTACTCATGAGTAATTTTTGGCAAATG
ATCCAGGTATTCCCTTCTCACTGGGTCATATGGACATTTCCCTTTCAGTTTACCTTCACCTTAGACTTAAACAGAAA
CACTAACGCCATTCCACAGAGAGGGTCGCTGAAATCTCACCCCAACTCTGAGGACTTTTCTGGTTTTGACAAAGGAT
AGAGTTTCTCTAGCCCCAATAGATCCACCCTGTCCTTATCCAGAGGTTGAAATTGTACTTCCATTGACATTCTATAG
GAATTGGTCAGGAGCTGTTAAATTTCCTATGACTTTTAGTACAATCTCAGATACAAACTGTTTTAAGTAATTGCTTA
TAAGCACAGGCTCTAGAGTTCCAACCCGTTTTGGAATTCTGGCCTTTCTGCTATCTATGTGCCCTTGAGCAAGTCTC
TTAAATCTTCTGCTCCATACATTTCTGATGTATAAAATAGGATAAGAATAGTGTCTATGTCACAGGGTTGTTATGAG
GATTATCTGACACAATATATGTGGGGAGCTTAGTGGGTTGCTTAAACACGATAATGGCTCAGTTAAGGTTGGCAATT
TTGATGATGAAAATGGTGATCTTATGGTTCTGATAGTCATGATATCTGTTAATTCATTCTTACATGCTTTCAAATTT
CCCTGAGAACTACAGAGAGAAGAATTAGACTCAGTCCCTGCTTTGAAGAGCTTCACAGTCTTGGAGAGAAAAGGCAC
AACCAGCCAGTAAAATAAGGGTTGATAGACTGGAGCTGTACAAGAGGGCAAGAACAAGATCCCTGGGGAATGGAATG
AAGCTATCTTACTAAGAATGGGGAATAAGTCAAGGTCTATAGACCATTCAAGGTTCTGAGTTACAACTAACAGAAAG
TGACTCTAGTTACTTTAGGTAGAATTATATAAATTACTAGAAGGATATCAGATACCTCATGGAATCAATAGGAAAGC
CTGAGAGCTAGGTTTAACAATGGGCAGAAACTATAAAGGAAGAGCAGGCTAGGCATCCAGAATATATCTACTATCAA
GCTATGAGGTAAATCTGAATTATTTTGCTGTCATGCCAGAGTCCATTGCTTTACCAGGAGCACCATTGCACTGGACA
TTGCTTGCTATCGTCACTGCAAAAAAAAAGTATCTAAATTATTTATTCTTGCTGTGTGTCGCTCATTCTACAACTAG
GGTCTCTGGCAGAAGCATCTGATAGTCAGAGGATGGATATTTTTTTCTGTAAAGGGGCAGTTAGTCGATATTTTTGT
CCCTGCAGGTCATGTAGTCTCTGTCACAACTACTCAACTCTGCTGTTTTAGGGTGAAAAAAACCATGTATAATATAT
AAATGGATGGACATGTTGTGTTTAAATAAAACTTTATTTACCAAGGCAGATGGCAGGCTGGAATTGCTGATGGGCCA
TAGATTGATGACTGCTGTTACAATACAGGTTTAGGCCACAGAACTAGGTTCTTATTGCCAGAGATCAGGGAAAATAA
ATAGCTGAATTTGTTCTTCATTGTAGTGGCAGTCAACACCTCGTGTCCTACTAAGTCCCATACAATGGTAGATTCTT
GAATCCTTGGAGAGATTTAGAAGCTATGCATTCCTGAATACACAAATGTTCAGTACAGCCTTCATGTGGTGTTCTCA
ACAAAAATCTGCTGAGTTTTTTAGTAAGCCAATTTGGGTTAAATCAAAATAAATTTGAGCTAGTAAGAAGGGCTATA
TTGGTATTTTTCTTAAATGGTACATGTATGGTAGTATTAGCTATGACCGCAATAATGCTGTGACACAAATCACCCTA
GAACTTGCTGACTGAAAACAACAGGCATTTCTGGCTGTTTAGGTTACTTGTACCTTGACTGACTCAGCTGGGCTTGG
CCGGGAAGCTCTGCTTCAAGATGTGGGTTAGTAGAGCTAGGTTCCATGATGCATGTTGGATTAAGGTCTGCTATATA
TGTCTTTGCTCTGGACCTAAGATGAAGGGTCAGTGACATGTTTTCATGACCAGTCACCAAAGTATAAACCTAAACCT
CCCAAGAACACTCATGGCCTCTGCTCCTGTGAAGTTCACTGACATTTGATTGGCCAAAGCAAATTACGTTGCCAAAT
CTGACATCAATAGAATGAGAAAGTAGACTCTTCCTACAGTGGAAGGGGGGAGAGATGTTAATATTTGCTGAACCATA
ATTCAAATTATTAGAGATAGTTAATAATTTATAACAGGATTGTTTTACAGCTAGAAAATGCCTTAGTGGTTTTGCCC
TGCTTCATTATTTTATAAGGGAGAAAACTATATTTCAAAGATGTTATGTGACTTGCCTAAGCACATACTAAATAGTA
CATCTGCGTATTTCTCGTTCAGTCATCTCTATTAAATGTCATAAGGTAAAACAGACATTACGCTTTAGATGAGAACA
GGAAGATATTTAAAAAGCCAGACTAATTTATGACTGCTCATTCATTATCTAACATACCTGCTTCCATACATTGTCTT
TCAGCAAATAGAATTTCAACATGTATCTCAACAAGTTACACATCATCAAAGTATTAAAAGCCTTATTTTCACGGACA
TTCTGATTGTTCACTAACAGTCAACACAGTCAACAGTAAATCCACAAGCACCATGAACGTATGGATAGATATGCCTA
TATGTAAGATTACTAGTTAACTTTTTGGGAAATTAATATTACTGCTAAATTCAGTTTTCATTGATTTTCGGGTGGGT
AACTAACTGCTTATAAAATATACCTATAGGAAACATGTAAAAACAAAGAATTTACATAAGATCTATCTGGGATTAGA
GAAACAAAGCTCTTTTCTTCTTTCAATGAACACTTAAGAAGGGCCTACTAAATGTCAGATGTTATACTCTATTGTCG
TTAGTTTCTTTCTGATACCATCTTTACTATCCTGTAAGCCATTAGTGAGTGTTTCCCACTTTTAGAAAATGACTTCC
CAAAGACTAAGAGGGTAGAACAGTTCAAATGCTATCATCAAAATGAAGGCAGTTTCTAGACTACAAATTGCTGTACA
GCCATAAGATGGCATTATTGTGTAACAACAAGGTCCCTGTCAAACATCAAGAAAGTAATGTTTTAAAATCAGTATTT
CCTTCTTGGAATTCTACTTAGTCAGATATGTTGGGTTGACTTACCCACCCATATAAGTAGTTAAATTCAAGATGGTT
CTGAGCTTTAGATATGATGTAAAATAGACATGAGACTAAGAGTATTTATTTCTATATTATTACATTTCTATAGCAAA
AAAAGAAACCTCTAAAAAAATTAGAGAAAGAAGTATAAGGGCATTTAATTAATCAAATGTTCTTATTAATAATATTT
CATAAAATAAAGGAAATGTTGAAAAATAAAATATGAACACAGTGTTGTATAAAGTAAGTCAGAAATAAGAGAACACT
TACTGTATGAATCTATTATATGAAACTAAAAAATTAGGCACTTTGGAGTTTAGAGGTCAGATAGGAGTTACCTTTGT
GAGTTGTGATTAACAAGGGCTGAAGCAGGCTTCTAGGGACTGGGTAATGTTTTGTTTCTTGATGTGGATGCTGCTTG
CATTGATGTGTTTCCTTTAGGGAAGTTTATGGAACCATACACTTGTGATGTGTACACATTTCTGTGTGAATGCTGGA
CTGAACTAAAATTTATACTTACATTTTAAAAATAATAAATATGTACTTTAAGTACAGGAGGGACATAAATCATAATT
GTTTTAATAAAGAAGGTGTAGCTTTAGGAAAAGCATACCGCATTGTTCTATTTTTGGCATTTCACCATGGACCCCTG
AACATTTTATAATTGCCCAGTACAGCCTGTCAGATAAATATGTGGGCACTACTGCTTTAGAGAAAGTATTTTCCAAA
TTTATCACCACCATTCTGGAACCACAGTTCCACAGGTCATTAGGATGCTGAGGGTGGGACAGTTGGAAGCTGCTTCC
CCAGGGATTTCTCCATTGTTCAGTTCTCATAGCAGTAAGCCTGCAGCATTAAAGCACTCACAGGCTCTGGCTGCCAT
TTGTGCTACATGAAGAAAAATGAGAAAATGAAAGAAACGAAAGGAGAGAAAACAGGAGTAGAAACCAAATAAATCAG
ATTCGAAGAGAAAGTCCCTAATCGTCTCTGTATTCAGACAGTAGGGAATGAGCATGCAATTCTGCAAGCCCATTCCT
AATGTGAGTTTCTGATAAGAATTTGCTATCTCAGTTGTAATCTTTCTACTGAGACATTCAATAAATAATTTTCTTTC
ATGTCAAGGTAAGGAAATAACCTCACATTACCCATATAAGGAAAGAATAGTATACTAATCCTCACGTTATCACTATA
GAATTTTATCTTTTAAAGTGGAAGCTAGAGGGACAGATGCATAATCTGGGAGCTTTTCCAGAATTGTTTGTTCCTAA
ATGAGACATTCAGCTATAGCAGCATTAGCAGCCTCTTCTATTTGTCACAATGCCATATGTAGTTGGGAAGATACATG
ATAAGCACATACAGGATGCTGACAACTCACGAAGGATTATCATTGCAGTCTTAGAATTAATCTCTACCATATTTGAA
GATTTTTATGCAAGACTGGATCTTCATTCCACAACCCAGGTATAGGTATATGTGCCAATACAAAATACTTACTGTTG
GTTTCTGAGCTTGGTAGAATTAGGAAGTAAGGGGTAAAAAACAAGCAAGTCCTGAACTTAAGAAAGTTGTTCATATC
CTATTCCATGGCCACTGTTTTACTGACTGGAAGAAAGGAATCCCGTGGATTCAGTCATGGGGACAATGCAGAAGAAG
ATACAGTAGACAGACAGAGAAGTACAAATTTCTATTTATGTTCCACTTTTCAAAATTTTTTATCTTTAAATGTTATT
TTTAACGTTTGTGGGTACATAGTGTGTATATATATATATATATATATACTGTATATATATATATATATACTGTATAT
ATAAACACACATATATAAATATATACTATATATAAACATATACTATATATAAATAGATTTACTATGTATATACACAC
ATACATAGTGTATATATGTGGGGTATATATATATGTGGGGTACATATATATATGTGGGGTACATATATATATATGTG
GGGTACATATATATATGGGTTACACATATATATGTGTATATATGTGGGGTACATATATATATATGGGGTACATGAGA
TGTTTTGATATAGGCATGCAAGTATCATGGAAAATGGGATGTTCATCCCCTCAAGCAATTATTCTTTGTTTTACAAA
CAGTAAAATTGTATTTTTTTAATTGTTTAAAAATGTACAATTAAATTGTTTTGACTATAGTCACCCTGTTGTGCTAG
CAAATAGTAGGTGGTGTTCATTCTTTCTAACTATTTTTTGTACCAAGTAACCATTTCAACCTCCCCCTCAACCCCCC
CACTACCCTTCACAGCCTCTGGTAACCATCCTTCCACCCTCTATCTCCAAGTGTTCAGTTGTTTTGATTTTTACCTG
CCACAAATAAATGAGAACGTGTGATGTTTGTCTTTCTGTGCCTGGCTTATTTCATTTAACACAATGACCTCCAGTTC
CATCCATGATGTTGCAAATGAAAGGATCTCATTCTTTTTATGGCTGAATAGTACTCTATTACGTATATGTATCACAT
TTTCTTTGTCCATTCATCTCTTGATGGACGCTCAGGTTGCTTCCAAATCTTGGCTATTATGAACAGTGCTTCAACAA
ACGTGGGAGTACAGATATCTCTTTGATATACTGATTTGCTTTCTTTGGGATATATACCCAGCAGTGGGATTGCTGGA
TCATATGGTAGCCCTGTTTTTAGTTTCTGAGGAACCTCCAAACTATTCTCCATTTTGGTTGTACTAATTTACATTCC
TGCCAACAGTGTACAGGAGTCCCCTTTTCTTCACACTCTCTCCAGCATTTGTAATTGCCTGTCTTTTGGATATGTCG
TTTTAATTGGGGTGAGAGAATATCCCATTGTAGTTTTGCTTTGTATTTCTGTGATGATTAGTGATGCTGAGCATATT
TTGATACACCTGTTTGCCATTTGTATGTCTTCTTTTGAGAAATGCCTATTCGAATCTTTTGTCTATTTTCTGATCTA
ATTATTAGACTTTTTCCTATAGAGTTGTTTTAACTCCTTATATATTCTGGTTATTAATCCCTTGTGAGATGGGTAAT
TTGCAAATATTTTCTCCCTTTCTGTTGGTTGTCTCTTCACTTTATTGATTGTTTCACTGCGGAGGAGCTTTTTAACT
TGATGTGATCCATTTGTCTATTTTTGCTTTTGTTGCCTGTGCTTATAGGATATTACTCAAGATTTGTTTTACCCAGA
CAGATATCCTGGAGAGTTTCTCCAATGTTTCCTTGTAGTAGTTTCTTAGTTTGAGGTCTTAGATTTTAGTCGTTAAT
CCATTTTGATTTGCAGTTCTCTGATGGCCAGTGATGGTGAGCATTTTTTCATGTGTTTTTTGGCTGCATAAATGTCT
TCTTTTGAGAAGTGTCTGTTCATGTCCTTCACCCACTTTTTGATGGGGTTGTTTGTTTTTTTCTTGTACGTTTGTTT
GAGTTCATTGTAGATTCTGGATATTAGCCCTTTGTCAGATGAGTAGGTTGCGAAAATTTTCTCCCATTTTGTAGGTT
GCCTGTTCACTCTGCATCAATTTTGCATCAATATTTTTTGCATCAATTTTGCATCAATATTTATCACATGGTAGAGT
TCAGCAGTGCAGCCATCGGGTCCCAAGCTTTTCTTTACTGGGAGAGTTTACTAAGGCTTCAGTTGTGTTACTCGTTA
CTGGTCCGTTCAGGTTTTGGATTTCTTTATGGTTTAATCTTGGTAGGTTGTATGTGTCTAGGAATTTATCCATTACC
TCTAGATTTTTCAATTTGTTGGCATATAGTTGCTCATAGTAGCCACTAATGAGCCTTTGAATTTATCTGGTATTAAT
TGTAATATCTCCTTTCTCATTTCTGATTTTACTTATTTGGGTCTTCTCCCTTTTTTCTTCATTAGTCTGGCTCAAGA
TTTGTCAATTTTGTTTACCTTTTCAATAAACCAACTTTTCGTTTTGTTGACTTTTTGTATGTTTTCTTTATTACAAA
GTCATTTATTTCTGCTCTGATCTTTATTATTTCTTTCTTCCACTAATTTTGGGTTCGATTTGCTCCTGCTTGTCTAG
GTCTTTAAGTTGCATTGTTAGGTGATTTATTTGAAGTTTTTTTCTTTTTTGATAGAAGCACCTACAGCTGTAAATTT
CTCTCTTAGTACTAGTTTTGCTGTATCCCATAGGTTTTGGTATGTGGTTTTTCTTTCATTTTTTGAAGACATTTTTC
AATTTCCTTCTTAATTTCTTCATAGACCCAGTGGTCATTTAGTAGCATATTGTTTAACTTCCATGTGTTTGTATAGC
TTCCAAAATTCCTCATTGTTGATTTCTAGTCTCATTCCATTGTGGTCAGAGAAGATGCTTGATATTATTTCATTGTT
ACTGAATTTTTAAGACTGCTTTTGTGACCTACTGTGTGGTCTATCTTTGATAATAATCTGTGTGCTGAGAAGAAGAA
TGTGTATTCTACACTCATTGAATTAAATGTCCTGTAAATATTAGATCCATTTATTCTATAGTGCAGATTAAGTCCAA
TGTTACTACTTTGGTTGAGCTTCTGTCTGGGAGATCTGCCCAATGTTGAAAGTGGGGTTGTTGAAGGCTCCTGCTGT
TATTGTATTGAGGTCTTTCTCTCTCCTGAGCTCGAATAATATTTGCTTTATATATCTGTACTCCAGTGTTGTATATA
TATTTGCTTTATATATATATACTCCAGTGTTGAGTGCATATATAATTGTTATATCCTGTTGCTGAATTGACCCTTTT
GTCATTATATAATGACCTTCTTTGTCTCTTCTTACAGTTTTTGTCCTGATATCGATTTGGTCTGATATAAGTAGAGC
TAGTCCAGCTATGTTTGGGTTTCCATTGGCATGGAATATCTTTTTCCATCCCTTTATTTCCAGTCTGTGTGTAGCTT
TATAGGTGAAGAGTGCTTCTTGAGGTCAACACAAAATTGGGTCTTATTTTTTCATCCATTCAGACACTGTATCTATT
TTTCTTTTCTTTTCTTCTCTCTCTTTTTTTTCTATCTTTCTTTCTTTTCTTTTCTTTCTTGAGACAGCATCTCACTC
TGTTGCCCAGGCTGGAGTGCTGTGACAGGATCTCAGTTCGCTGCAACCTCCGCCTCCTGGGTTCAAGAGATTCTTGT
GCCTTAGCCCCCCAAGAGGCTGAGATTACAGTCACCCACAATCACGCGTGGCTAATTTTTGTATTTTTAGTAGAGTT
GGGGTTTCATGATGTTGGCCAGGCTGGTCTCGAACTCCTGACCTCAAGTGATCCACCTACTTCGGCCTCCCAAAGTG
CTAGGATTACAGGCGTGAGCCACCATGCCTGGTCGAGACACTGTATCTCTCTTGATTGGAGAGTGTTGTCCAGGAGC
TGAGCCTAGAAATGGGTCCTCACAACACTGTCCAGTGCCCTATCCTACTGTGACTAAGCTGGTATCCAAGATGCAAG
ACAGAATCCTCTTTACTTTTCACTCTCCTCTCCTTAAGCAAAAGTAAGGAGTCACTTTTGTTACTGTGGGCTGCACT
GCTTGAGGTGGGGGAGGAATGGTGCAAGCACTCCCTTAGCCATGCCAGCTGGTGTCTCCCTAGGTCACATGCCACCC
TAGTCCTCTAGCTCTTAGCCCAGGCTATGACTAGAAGTTATCTAGGAATTGCAGTTCGTGTGTCCCAGACTGCCTCT
CAGGTTTACCTGGGACTCCAGAGCACTTTGGCCCACAATGGTGAGGCTTGCCAAGAAACTTGAGTTCTGACTGCTGG
GATGGGCGATTCCCTTCTGGCTGGGGCTAGTTCAAATATTCCCTCCGTGCACAGGCACTGACTGAGGCCAGCATGGC
TTTTTTCTCTACTGTGACAGCACAGCAGTTAGTTCAATGTAAAGTCCCCCAGTAGCTGTACTGTCCCTTCCAAAAGT
GCAAAGATTCCCTCTCTGCACTGCACGTCCAGTGCTGGGTGATTGGGAAGGTGTGGTGTTGGTGATTCAAGACTGTG
TCTCCTGCCTTTCCTCAATGCCTCTTTTAGCAAGATGAAGTTAAAACCAGGTACCATGATTCCGTACCTGGTTTTTG
GTTCTGGTGACAGTGCTTTTCTGTCTGCTCATAGTTGTTACAATTTGATGTTTCAGTGAGGGAGATTAGTTATATAG
GCTTCTATTCTGACATCTTGCACCACTCCGTCTCACTTTTTTGAATTGTTGAAACCATGGTTTATGGGGAAATGTTC
TGTTCTGGTTTTTACCCTCAAGATCTTCTTTGAAAATCATGATTTACTAAGTAACTAGTTGGATAGAATTCATTTTT
AAGAAAATAATGAATAAGTAGTTCCCTTAGATATAGCAAGCAATGTTCTTATAAACTGACACACCAAAACACTTACC
AGTTCACCTAAACACTGAAAGTATTTAAAAATTTATTTTTAACCTTTAAGTTTTTCTCCATTTGAGTATCTTGTGCA
CTATACGCTCTCCTACATATGTCTACAATCTTTGGTCTGATTTATTTTATTGGTTGTTGAATGTCAGAAACCACCTG
AATTTTGCTTGTATATATAATGCCCAGTAGAGTGCACATTGGTAATCAATACGACTTGATGATAATAATGGTAATGA
ACACTATAATTTGATAGCATCAACTTGGCATGTATGCCAGAAATAAACCATCATAAACTTCGAGAAAAACAAGTCTC
ATTAATCTGTCATTTATTATAATCTCCCTCCATCAAGCGTAGCCTCTATATACTGTCTTTTTTGCCATATGTAAGTT
TCTTCTTGTATATTTCAGATTAGAGCTTGCCTCCTCCCACACACTATCTTCTATTTCACACTGCTCTGTTCAAGTGT
TTATGTTTCTGCTGAAGGCTGTATAATCTAAGATAATCCCTAGCTATTGTAAATATCATTCACTGGCCAGTCATTGC
ATTTTGCACACCACTGAGAGAGTGATTGTGAGCCAAATAGAGGAGATTTTGTTTCCACCCAGCAACCCAAAATGAAT
TGTGTGATTTGACTATTGATGCCAATTACATGGAAGTTTTACTACCAGAGGCAAAGCTTTAAACTGCAAAAGATTAA
CCCCATATAGTTGGTGTCATGAGGTATTTAATGTATATTGGACTATCAAAGCGTCTCCTTACAACAAACCAATTATC
AATGAATAATATTCTTATCTTATTGTGACTTATTGAAACTCTTTAAAAAACATGAACCATTCAGCACACTATACCGT
CTAAATTAGATATCTAAACTAGCCTTCATTTACATTTCCTTGTTTAGTTTTAAATATAATTTCTTCTGAATTCTTAG
TTGTTAAGAGCAGGTGGTGAAAGAATTTAAAGAAGAAAATTTGTTGTATTTATTAAGTGAAAGAGCTTTTTGAAATG
CATTAAATAGAAGTTTGTTTCTGCCCATCAGCTTATCTTCTGCATATTCCTATTTATAGTTCAAAGCACTTAAGAGT
TGCAGAGTGATTTGCTTTTTATGATCACCGCTTCATCCAGGATCATTTGCCTTATATCTTCAACATTTTCAGATTCT
TATAAATCTTCACAGTTCACTTAGAGGTTAGTTTAAAACTTTCTTCAGATAAGAAATTGACTAGATATAAAATTCAC
ATAATCTCAGAGTTAGCCATAGATCAATATCCCCAGTCTTTTTCTCAAAGTAATTGAACATTTTTGTTTCCCAGGAT
AGTTGTATACAAGCATTTTCATCCTCACCATAGATGATTCTTGTTCTTCAGTATATGAGGGGACTGTATTTGCCTGC
TTGTTATCTAAATAAACATTTTTATTATTGTCAAAGGAAAAAGACTCTAATACAGAAAGAAATTTTGTGTCCATAGG
ACTAGCCTCATTTTGGAGGCTGATATTAATATTTGACTTTATTCATTGTGTATTTCTTGGTTTTGTCATTTCACAAA
TTTCCCTTCTCTAAATATGCATTTTTTCTCTACCTGGAATAGATGAGTTTTAAAAGACATTACTGTAAAATAAAACT
GTGCTAAATGTACTTTTCCCAGAAACTCTCTTTATTTTATCAATTATAATTTGCTTTGGCTAAGTGTTACAGAAGCT
CAAGGTAGAAATGACTTAAATAAAAGAGAAGCCTATTTCTTTCTCCTGAAAAACCCTGGAAGTTTGCAGCCAAGGCA
GATATGTTGGCCCCGCTCATTTCTGTTTTCTACTGAGTGAGTTATCTTTACTTGGTGGAATTTCATCCACATGTTGC
AAGATGGAGCTCTCATGACATCCACATTCCAAGAAGGATGTAAGAAGGCCAGAAGAAGGGTCAAAGGGTGCATACCA
ATTGTCTTTTAGGGAAGTTTCGTGGGAGCTGCCATGTTATATTTCCACTTACGCTTCATTGGCCAGATGTCTGTCAC
ATGGCCACATCTAAGCTGCAAGTGGGGCTGGGAAATGTGTTTATTTCACATGTGCTCTGCTAAAAACTGTTCAGTCA
TGAAAAAAAAAAAAAGAATATTGGAAGAGAACTAACAGTCTTCAACATGTATATTTAAAAAGAGCGTTATTTTTCTT
TTACTTATTTATTTTTTTTTGAGTGAGAGTGTTGCTGGGGCTGGTCTCAAACTCCCTGGATGAAGTAATCCTCCCAC
CTCAGCCTTCTGAGAAGATGGGATTACAGAGGCACACCACTGCACCCGGCTCCTGTTTTGTTTTGTTTTGTTTTGTT
TTATTTAAGGAGCCCTAAGAAGAGTGTAAAAAGGGCAACTTTGTCTTTAGTTAACCCTTCCCTTTTTTTTTCTTTCC
TAATTTTATGAACTCTGATTCAGCCCGCATCTTACTCTGGCTTACTAAATTCTGCCAATATAAGAGTAGAAGTTTTC
ATCATTTCCCTAGGTACCTACTCTTCTGTTTCTCCATATTCTCAAAGTAATTTCTCATTCATCAACTTTTGTCTGAC
TTAGATTTAAGCTACTGCTTAGGAAATGTGAGAAATGGAGAATTTGGTTGTCATAGCGCTCCTACTTAGAGGGCTAA
TGCTTTCCTAAATTTTACTAAAGTTGGATCCTAAGTTATATAAGTTACCTAAATTTAATTAGCAAGTTTTGCTTACT
AATTCACTGACTAACCACTATAAAACAAGGACCTAAGTTTAAAGCTGATATTATGACAATGTACTGAAATTTCAAGA
CATTACTTTTTCTGGACACGTAGGATGGACCTTGCATAAGATAAAAGAGCTTATTTTTAGGCACACCTATGTGCCAG
GCATGGGTTCAGAGCCTTACATATATCATTTCATTCTCAAGAGAACCTTAATTTTAGAGACGAATAATTTTGAGTTT
TAGAATAAATAATTGTCTTAAGCTCACACATGAAGTATGTGCCAGAGTCTATATCGTGACCCCAGTTATGAATTCTT
TTCCACTACATAGCCTGCCTCCATGGCATTTCACATAGGATTTCTTATGCTCAAGGATTATGTTCTTCCTTCCAGAT
CTATAGACATCAGTAGAAACAACACAAATACGTATAATGACTTTTTAAAGTCTCATATACAAGACCAAAGTACTTTA
AAAAGATGTTTAAATTTTTGAAGGTGATATCTTTATGTTCGAGATCAGTAGCAGAAATGGATTGTAATTCATTACAA
GATGCTTAGCAAAGCCAAGTGGTAAAGGATTTTCTGATGTGCTTGAGGAGTCCTGACTTGCTCCTTAGCAAATTCCT
ACACACCATTCAGCATTCAGTGCAAATACCACCTCTTGGAATCCTTCTCTGTTCTTGATCAATTATTCCATTGTCTG
GATTCATAGAACTCTCTGTTTACCTATTTATCTTACAGGTTATTATCATGAGTTTATATGTCTTACATAGCTGTTTG
CAACATGTTACCATTCAGTGGAGTACTTTTTCCTACAGGGAAGAGGCTGTACTATATACCTTTTGTCACCCCTAAGT
GGAATGCAGTATGGTTTCATGTTAGATGTTCAATACATGTTAATATTTTATTGATGAGCCCTTAAATGTATATAATA
AGCTTCTCACTCAAAAGCTGTAATATTCTAGAATGAATAAGTTTCTTGAGAAACTCTTTGGTTAACAGAATCTCCAT
TTCCATCATAGTGTATTCAATAAATTATTAATGCAGGGTAAATTTCCTTATATTTCCTTTCATTTCGCTATACTAAG
GTCAAATCATGACTTTTAGGTGTGGATACAGCAATATATAAAATAATCTAAGACCTGGATATATGTTTTTCTAATTA
GGGAATTGGAGATACATGAACAGTCTCTCATAGAGAAAAATATATCCATTAGTCACTAAAGTCAGTATAACATTCTG
GTGAAAGGTTTGCTTGCTTGCTTGTCTGCTTACTTGCTTGTTTCACCTTCTATTCTAGGAGGATGTTTGTCACAATT
ATGAAATTCTAACGCTTTAAAAATCTGTTTACCCTATGAAAGTTGCAAATTCTTGCCTGTAAGGCAATTTTCACAAG
AATTATTTTCATATTAGTTTTAGTTTATCCGTGGATAGTCTCAAATTCCTCTTGAAAATATCAGTGTTCTCAAGGGA
AAACTGCCCATTACAAGCCACAGATGTGGTTTTTTGAGAGATTTCTGCCTGAAATACAAGTTTTAAAATATTCTTTG
TAGTTTCCTAGTATGGCTAGATACTTGTAGGAAATGACAATTTAGATGCACATTTGAAAATTCTTAGCCTTTGAGTA
ATTTATATGTTTGAATAAATTTAAACTATCTTTGTTCATAAAAAGGCAAATTTTGTCTGTTTGTCTTGAAATGCTTT
TTTAGTTTCTTCTTCAGTTCTTTCACTTGTCTTTCTGTAATATTCAAAATTATAGCATAGTACTTGAAAACCAAGGA
TGGTCCAGTCTGTGTCCTTTGTTTTGAAGGAATTTTGGAATCATCAGAGATATAATTCTGGAATTTCTTTGGGTACC
TTCCATAAAATAGTAGCATCCTGATAAGGACGTAGATGCTAACTTTTATTTTTATCTCATTTTCTTTCCCCTATCTC
TCAGTAATGTAGGAGTGACCCAAGGTAGCTGTTTATTGGGCCCTCACCAAAGTCAATCAATATTAGTCAGCTACAAC
CACAATAATGCTGTGTGACAAGCATACCCCAAAAGAACAATAAGCACTTATCCTCATATTCACAGGCCTACTTTGAG
TAATCTCTGATTCAGAATCTGACTGTAGGTTGACCAAACATGTCTGATCTATGTGTCTCATTCTGGGGCTCAAGCTG
GAATGGCAGTAGATATTTGGGGCACATTCCTCCATGGCTGAGTTCTAAAGATCTCAGAAGGGTAAGCAGAAACACAT
GATGGTTCTTAAGACCCAGATTTATAACTAGCGTTCTCTCTTCCATCCATAGTCAATTGTCAAAGCAAGTCACGTGG
CATATCCAAAGATCAGGGATAGAGAAATACACTTGGCAGTTAGTGAACCCATTTCAAGAGTGTGGGTGTATAACACT
ACTACAAAACAGAACTGAGACTTGCAACTTGAAATACCACACCATTCTAGTAGAATTTTATTACAGTATTAATTAAA
CATTTTTGATAAAACACAGACAACTCTGTTAGAAAGCCCAACTTACAATTTGTTGATCCCACTTGGAAAGGTATTCT
GAATCCATTTTTTCACAAGTTTTCTTTCTAATTCAATGTGTTGGGGTACTTTTATTTTACCCAGAAAAGTAATATTA
TAAAGCATAAAGTTAATGCCCCCTGTAAAAATAGAATACTGAAACAAACTTTAAGTCACAATTTTATTATTTACTGA
CTTGAGAATCTCCTTGTGCTTCAGCACACTTGGTAGTTGGATTTCCAAATAGCATCATGACTTTCCATACCCAGCTG
TCTGCTGCTCAAAACTACCATAAGCTGACTATCACAGCTCACAGGATACCCTTGAAGAAGACCATCTGGTTTATGTC
TCTTGGCAAAATTTGTTTAAAAACAAGCAGCTGCATGACAAAGTAAGAAAGAGTCTCTGTCTAGCTGTGGTAAATAA
GTAGCTTTGCAATACTCATAATAAAGCATTTTTCTAATCCAAGCCAACATCTTCAAAAGGAAAGCATTATTTTCTTA
CAACTGAAAGAAAAATCTACAGAATTCAGTGATGAGGGAAGGACTCCATTAAATTTGTTTCAGCAGCTTGAGATAGT
GAGAAAAACAAAACAAAATAAAAACCTGTTTGTGATTAGATACAGTCTTGTAATAACTAGTTTTAAAAGTATTTATT
TAGAATCTAATACTTGGATATTATTTTAATTCTTTATATTAAGAGATTTACATTTCATAAAAGCTTGTCCTTTGAAC
CCTAATACTCATATTGTGATGGTGAGCCGGAATGAGTAAGGTCACTTACTATATAGTCGGTATATAATAATTCGTTG
AATAACTCTCTCTCCTCCTGTTACTGGCAGTGAATCCATACGGGTCTTCAGCAACCTTAATTCATGCCTCCTCAGAA
GAAAGAATTCGACCAAGGAGGCATAAGGCAGAAGAAGAGACTGAGGCAAGTGTTAGAGCAGGAATGAAAGTTTATTT
AAAAACTTTAGAGCAGAAATGAAAGGAAATAAAGTACATCTGGAAGGGGGCCAAGTGGGCGACTTGAGATATTAAGT
GTCCTGTTTGAACTTTGATTTATGGTTTTGTATTTTGGCATACTTCCATGGTCTTATATACCTTCTCCCCAATTCTT
CCCTTGGGGTAGGGGGCTATCTGCATGCACAGTGGTCTGCTGACACTTGGGAGGGGAGCATGCACGGTGTATTTACT
GGAGTTGTATTCAAGAGGTGTTCCCTTACCAGTCAAGTGTTTCCAGAGGAAGGTCGTATACCAGTTACACTATGACA
TTTTGCCTCTTAGTGCGCACGCGGGAGCCCATTTGCCCAACTCCTGCGATCTTATCGGGAAGCTGCTGATCACCAAT
TTCAGGTGTTTTTATCTATTGGGAGACTGCCTTTCCCTGGTATTGGCTGTGACCAATTATTATTTTAAAGACACAGT
TTAATAACCACCTGACTGACCATCACTTGATGGTTGCCTGACATTCCTGGTTGGGGTGGCGGGGGTGGGGGGTCTCC
TACCCTGCTCATATCTGACTAGCTATCTACTGTAACACTACTATTAGGTATGTCTGTATCCCATTACACTCACTCTC
TATTACAAAAAAGAGTACCTTATTCATTTTTCAAAAGAATGAGTGAATTTTCTGCTCCAGTTAAGTGCTTTTCCTTA
CCTCTATGTAGAGTGTCAAACTAGCCATGCTTAAGGATTGATTCTGGTATTAATAATATAAATTCTTGCATTATGAG
TGTCTCAGTTTGGGTTCTTACATAAGCAAATCCTGAGACAAGGATGTGAGTTCAAATAGTTTCTTTGGGCAGTGATT
CTAGGAAGCTGTGGTAGGATGTTGGAGTTGTGAGAAAGTTAAGAGAAAGCAACCTGTAAAGAGTATGTTATCAAGCC
AAACACCACTGTGAGAACTAGGGCTTAATTCCCTGGAGAAAGTCTGGGAAGGCATAAAACACTTGTCTCAGAGTTAT
CTTAGACAAGGTGCAGGAGCTGAGGTGTTTATATTTCAACTCTTGTTAGTCACTTTTAAAGGGCCGCTCAGGGTGTC
AGGGTATTAATTTCCTCTTGCAACTACTTCCAACCTGTTTTTGCATGTGGTTGTAGCTACTTTTTCTGCGTCAAAGA
TAGACTTTAGGCTAAGACATGCAGTTTCCGGCTGTAGGACATCAACCAGAGTACCCTGGAGTGTCCAGGCCCAAGAT
AATGGGTGAGCTCCACCTGGATCTGTGGCAATGTGAGGGAGGGGCATTATTACCCTAGCAAGGGCCACTGCACTTCC
CTATCCAGCTGGTCACTCTTCTGCGTATTTGTGTTACTTTTCTGACTCCTGCATTCCTCTGGATCCTGATTCACCAT
CAGCTTTTGTATTCTGAGGCTTGTGTGGCTGTGTCTTTCTATTTCTTCCTTATTGACTTACTTGTTTAATCTTTTGC
TAATGTATACCTTTGATATTCCTATGTACTCACCTTTGGTTAGCTCTGTGAATTATTGAGTCTTGCTTTACAGCTTT
AAAAAGATCATAGCCATGGTTTGTTTTTTAAGTGTGATTCATAACAGTTAGTGACAAAATTAGAAGTGGAAATTAAA
GGTCACGTGCACAATCCTACACAAGCCAATTTCTTGAACGACATAAAATACTTGAATTTGTCTTTTACTATCACCAA
TATAGAAATAATTTGGGGGTATTTCTCAAAGTATTGATTTAACAAAACTTTATTTTTGTGTGAAGATTTTTTCAAGT
CTCCTGGAAATTATTCAAATTATTGTCTTTAAAGTCAAAGGAAAGGTTAATATTAGGGTTACTTTTCTTTGGTCACA
GAATTGCTGGAGGCTTCTATGACTTGCTTTGAAGCAGCTGACTTTATGCACAGTTTTGGTTAGAAATTCACTACCAA
TTCCAACATTTTCAAAGTCCTAAAAGACCAAAAGTACAATTCTTGAAATATCCCTAGAGAAAGCAAGAATAAATTAG
TTTTCACTAGAAATGAAGACAAATTTTTCTCATTAGTTCATTTTGCCTCATGATCAGAATTCTTTGCCACATGAAAA
TATTTTGGTTAATAGGTTGTAACATAAAGAGTAATATGAATAGAGATCTAGCCCAATTAGATGACAGAAAAAGAGCA
AAGCTTTTGAAATGTATAAAGAAGAAAGTTTAAGAGAGACATGAACAAGGCTGGATGTAGTTTAAGGTTGAAGAACA
CAGAAAAATGAGATAAGTGAAGTTAAGTGAAAATCCTTCCCTTACTGAGAAGAAAGAGAACTCAAAATTATGTGAAG
GTTTTTGTTTTACTTTGTTTTGCTTCTTAATAAGGAAACAACAGTGGCAAATCTGGGGCATTAGGAATGGTGGAACT
TCTATAACAGTGATGTTTCAAAGGTCACAAAGGAAATTAAATTGTAGATTGGGTTTAAAAGCACTGGAATTACTTCC
AGCCCCATTTTTTCAGGATTTTGTATGTAGCAGGAAATAGATCACCGCATAGCTAAAGGGGAGAATTTAGGTTTTAA
CTGGTCTCAGTGCAAACTGCTTCATAGTTCCTTTGCATTTGGTGGTGTATGTAAAACATGAAAGGTATAAACATTTA
TTATCATAACTTTATTATTATACAACTATTTATTGGCTGCATAGGACCATGTGTCTTCTTGCAGGTATAATCAAAAA
TAAAAAGACAGAAATGTATCTTATATGAAGGCTGCCATTATCGCTCTTATCAATGGCCATAAAATCAGATTTCTTAC
ATGTACAACATATGAAAATATATTAAATATGAAACGTTTTCCATTAAATAATTCTGTAAATGATTTTCATAACATTT
CTGTCCATGATGTGTAAATCTGTAGATCAAATACTGCAAGTGTACAGAACTTAAAATGCTTTGGTCAAAAAAATTCT
CTTATTAATATGACAATGGCGTTCAAAAGTAAAAAGGTAAAAATACAGTTTAGCATTAATAAGTAAACTCAGAAAGT
AAAATATATTGATCATACAGTCTGTATTAGTCTGTTCTCATGCTGCTCTAAAGAACTGCCCAAGACTGAATAATTTA
TAAAGGACAGAGGTTTAATTGACTCACAGTTCCACATGGCTGGGGAGGCCTCAGGGAAAACTTACAACCATGCCAGA
AGGGGAAGCAATCACATCCTTCTTCATATGGTGGCAGAAAGGAGAAGTGCTGAGCAAAGGGGGAAAAGCCCCTTATA
AAACCATTGGATCTCATGAGAACTCACTATCACAAGAACAGCACCATGGGGATAACCGCCCCTGTAATTCAATTACC
TCCAACTGGGTCCCTCACATGACACATGGGGATTACAGGAACTACAATTCAGAATGAGATTTGGGTGGGGACATAGC
CAAACCATATCATTTCATCCCTGGCCCCTCCCAAATGGCACGTCTTCACATTTCAAAACACAATAATGCCTTCCCAA
CAGTCACTCAAAGTCTTAACTCACTCTAGCATTAACCCAGGAGTCCAAGTCCAAAGTCTCATCTGAGACAAGGCAAG
TCACTTCTGCCTAGGAGCCATAAAATCAAAAGCAAGTTAGTTACTTTCTAGGTACAATGGAGGTACCAGCATTGGTT
AAATACACCCATTCCAGATGGGAGAAATTGGCCCAAACAAAGGGGCTCCAGGCTCCATGCATGTCAAATCCAATGAG
GCAGTAATTAAATCTTAAAGCTCCAAGATAATCTCCTTTGACTCTGTGTCTCACATCCAGGTCACGCTGATGCAAGG
TGGGCTCCCACAGCCTTGGGTAGCTCCACTCCTTTGGCGTTGCAGGGTACAGCCCCCTTCCTGGCTGCTTTCACAGG
CTAGCATTGAGTGTCTGTGGCTGTTCCATGCACACGGTGGATCAAGCCCTCTTCTCACAGCTCTACTAGGCAGTGCC
CCAGTGGGGACTCTATGTGGGGGCTCCAACCCCACATTTCTCTTCTGCACTGACCTAGTAGAGGTTCTCTCTGAGGG
CCCCATCTCTGCAGCAAACTTCTGCCTGGATATCTAGACATTTCCATATATCCTCTGAAATCTAGGTAGAAGTTCCC
AAAGCTCAGTTCTTGACTTCTGTGTACCCACAGGCTCAACACCACATGGAAGCTGCCAAATCTTGGGGCTTGCACCC
TCTGTAGCCATGGGCTGAGCTCTATCTTGGCTCCTTTTAGCCATGGCTGGAGTGGCTGGGATGCAGGGCACCTAGTC
CCTAGGCTGCACATAGCAGGGGGGCTCTGGGCCCGGCCCAGGAAACCATTTTGCCTTCCTAGGCCTCTGGTCCTATG
ATGGGAGGGGCTGCCATGAAAACTTCTGGCATTTCCTGGAGACAATTTTCCCATTGCCTTGGTGATTAACATTTGGC
TCCTCGTTACATATGCAAATTTCTGTAGTCAGATTGAATTTCTCCTCAGAAAATGAGTTTTTCTTTTCTATTGCATC
TTCAGGCTGCAAATTTTCTGAACTTTTATGCTCTGCTTCCCTTTTAAACATAATTTCCAATTCCAAACCATATCTTT
GTGGATACATAAAACTGAATGCTTTTAACAGCACCCAAGTCAAATCTTGAACACTTTGCTGTTTAGAAATTCCTTCC
ACCAGATGCCCTAAATCATCTCTCTCATGTTCAAAATTCCACAGATCTCTCGGACAGGGGCAAAAAGCCACCAGTCT
CTTTGCTAAAGCGTAGCAAAAGTGACCTTTACTACAGTTACCAAGAAGTTTCTCATCTCCCTCTGAGACCACCTCAG
TCTGGACTTTATTGTCCATATCACTATCAGCATTTGGGTCAAAGCCATTCAACAAGTCTCTAGGAAGTTCCAAACTT
TCCCACATCTTCCTGTCTTCTGACCCCTCCAATTCTCTAGGAAGTTCCAGAGTTTCCCATACTTTCCTGTCTTCTTC
TGAGTCCTCCCAACTGTTTCAACCTCTGCCTGTTACCCAGTTCCAAAGTTGCTTCCACATTTTTGGGTTTCTTTATA
GCAGTATCCCACTCTCTGTGGTACCAATTTACTGTATTAGTCTGTTCTCATGCTGCTATAAAGAACTACCTGAGACT
GGGTAATTTATAAAGGAACGAGGTTTAATTGACTCACTGGTTTGCATGGCTGGGGAGGCCTCAGGAAACTTACAATC
ACTGTGGAAGCAGAAGCAAACACATCCTTTGTCACATGATGGCAAGAAGGAGAAGTGCCGAGCAAAGGAGGAAAAGC
CCCTTATGAAACCATCAGCTCTCATGAGAGCTCACTATCATAAGAACAGCAGCACGGGAGTGACCACCCCCCATGAT
TCAGTTACCTCCCACTGGATCCCTCCCATGACATCTGGGGATTATGGGAACTGCAATTCAAGACGGGATTTGGGTGG
GGACACAGCCAAACCCTATCACTGCCTTTAAGATCTATGAATTGTTTTTCTACATTGAAGAAATTTGTAGACATTTG
TTTTTTATTCTCTTTTTGGTTTGCTGAGAGCAGTAGTCTCTTGGAAGTGAGTTTCATTTACATTTTTTCCTTTGGGT
GTCAAATATATAAAATATTTTTTAAAATCAATGGCTGAGGGATAAATGAGGGTTGGTAGAAAGGGAAAGGATAAGGG
TTGTAGTTAACGGACCATGTTTTAATGTGTCTATAATTAGATGGAAGTTGTCTCTTACCATTCAACATCCTGGATGG
CCTTCCTATAGAGGACATTTTCATCTTCTTTTGTCGTCCAGAGCTAGCATATGTGTTTCATGTCTTTAGATAGTGAA
AATCAATTCAGATTTTCATGGAAAATGCTCTCTCTTCATCCTAAAGACAGAATTGTCACGGCTATTTCGTTTATTGA
AAAGTGAAACCTGCTACAGTTAGCATATAGTTAATTCCATATATGTTACCTGTATCATTTAATACATTCACAAAGTA
AAAAACCAAATTTTGCCAAGTTCTTGATAGTAACAATAGTAAAATGGAATTTCTGTTGGCATATTTTCATGTTATTC
TGAAATGCTGAATGGATTTATTAAGATTGATAACAATATGTTGTACCTTTATGTTCTAAAATTTAATAACAGTTATT
CTTCCAAACATGCCAGCGTCCTCAATAGTTGACAGTCTGCAAAATATTCTATTATGCAATCAGCCATATCTCCTTCT
TGAGCTTTCTTTAAGTACTTGTATCCTGTATTTTTCCCTGAAATCTAGCAGTAAGTTGAAGAAATGATAAACAAAAT
TGTATCATTGTATATAATGGCTATTTATAAATACTGATGTACATTTTCATAATGTTATTTAAATGATTACTTCAGAT
ATGTATTTCTGTTTTCTACATTTTAACTTCTCAACATATTAGGTTGTTATCTTAGATTGGATTTTCTAAGACATAGA
CTCTAAGACAGAGATTTCCATGCAGATGGTTTCTTAGTGAGTGCTTTTAAGAAAAATGCCTGGGAGGGAGTAAAGGC
AGCAGGATTGAGGAGAGAGAGGAGTTGAATGATGATGCAGTGAAATGGAAGATCTTATCTGGTCCCATGTGGGGTTG
GGATGACCATTTAGAGTATTACCGTATGAAGCAAGGAAGGTGGACCATTGTATCCCTAATCCATTAGTCATTAGATG
TGAGCCTCCCAACTACCAGCTTCCGGGCAATATCATCTTGGTCAAAGAAGTGCCTGTCTGCTGAGAGAAATTTCTTG
GAAGAGCTTAGGTATGATCTGTTAGTAGATAACAATCCCAGCAACTGGGAGAATGAATGCTTCATTTGTAATTGGTG
AATCTCGGTGGCACACCACAGTGTTCGCTACGGTAATCTTTATACTTATTGAGACATATATTGTTTTTAAGTAAAAC
TTGTATACCTAGTAATGCATTTAGGTAGTGTATTGGCCAAGCTAGTCCTTTTTGGAAACAATTTCATTGTGGTATAA
ATGATATACAATAAGCTACACATTTAAATAGTACAATTTGATAAGTTTTGACATATGCAAATACCAGTGAAACTATT
AGCACAGTCAAGATAATAAACATATCCAATATCCTCACAAGTCTGTTTTCGGTAATCACTCTCTCCTGCCCCTCCCT
TGATTCCCAGGTTACCACTGATGCTGACTGGCATTATAGATAAGTTTGCGTTTTATAGAATTTTATAAAAGTAGAAT
CATGAAGTATGTACCTTATTTCTGGTCTGCCTTCTTTCACTCACAAACTACTAACTTTCATTTTGTGAATAATATGA
TCTCCGTGTGATTACACACATTAAGAAAGTCAATAAAAACATTGTAATTTGATTCCGATACTAGTTTATGGGTCTAC
ATTATGTATTTAAAGATACTATCATGTTTGGATTAGCCCTTAGGTATCAAGTAACCCTGGAGTTGAGGCAGTTACTG
TTTTATTTGCTATCAATCTTTTTATAAGCTCACCTTCCACTAATTACCCAAAAATATTTGATTTAATACCTGAGTAC
AGAAAAAGAACATATTCACAATGTTTAAATTTTATAATTTAATATTATAATTTACAACAGTTTAAAATAACTAGTCC
TGAAATTGAGTATTTCTCACCTAATATTCTGCTCTATTTATTACCCACTCCACGAAAAAATTTCCCAGAAAAATCAC
AAACATGTCTCTTGAGTGTATTATTACCCAACCCACAGAAGAATTTCCAAAAACAGTCGCAAACATTCTCTCTTGAG
TGCAACCATTTTCTCGGGCCAGCCATTCATTGAAACAAAATAGCCTGTATAACTTCTGTGGGTAGACATTTGTAGGT
TGTTCTTTTGAGGCATTAGTGCTATTGCATTTAAGATAAAGAAATAACGAATTCACAAATTTTATAAAGTTCTCTTA
AATTTACCAAGTCAATAGCACCACCACTTTGATTCCTATAACCTGTGACTTAGTTAGGCTTCTCAATTCCAAGAACA
TACCACCATTATTTCTTATAATGTCCTCAGATTAAAACATGTGTTGTCCTTTCTTTATACCTCCCTGCACTTTAATT
GAATATTTTGCCATTTACTGGATTCTGTACACTTTTTATGTTTTCATGCCCTCCCTTTTCATTCTCCTGGACATCAA
TCGCAAGTCAGTAGATACCTTGTAAGAGATCTCTCTCCACCCAAATCACTGTTTCTTTTGAAAGAGTTAACCTTTCT
GAAGCAGCACTCCTGCTTCACATTTCCCAGCACAGGAACCTTCAGTGTTTCTCTGTTGATAGCTTTGATTTCAAACG
GGTAGCCCCAGGAGAAAAGAGAGCTGAAGGATATGTTTTGCTTGGCTGCTTTGTTTGACCTGCAAGGAACATTAAAA
GGCAATGAAACCGGGCAGAGACTGGACTGATCAACACCATCAGTTTAGCACTTATTGCTATACTTTCTAAGGCTTTA
CAATTTTATGTGTCCCACGTGTCCCCTGAAGGCATTTGATTTTCTAACCTCCTAGGCCAGAGGCTAACATCCAGAAT
TCATAGTCTGGCACTTATAAACCCTAACTTAGCTTCTGTCACTTGTATCTCTATGCAACTCTACAGCAAAATGTACT
TATTTTCTCTAAAATACCCTTATTCCATACTTCTTTGGAGTGTAATTTTATATATAAAAGTCCTCACCACCTGTAAA
CATTAGGAAGCTTTGATTCGAAGAAGCAGAAGCCATCTCCATTTAACTGAAGTGAAATGAAACTTACTGGAGGGATA
ATGTATAGTTCTTGGAAATGAAGGAAAAGAAGGCAACTGTGGCAGAGGGAGGGCAGCACTGAGAGTCAGGAACTCCA
GGGAAGTTACTTTTTGGATGCTGTCATTAAAATGCTTCAGCTTCAAATTGTACTGACTCTACCTTTCTCAATTCTAG
ATTCAAATTTCTAAATGTAATTGGCATAAATTAGGAACTTGTCCCACCAGTTTATAGAGTTTACAGAGCACTATGAT
TATAGGTCCCCCTAAGATCATGCAGAATGAGGGAAGAGTAATTTCATCCAAGGAAAAGCAGGGTACCATTCACCAAA
GAGTGGAGATACTTATTCAGAACAATTAATGTTAGATGCCACAAAAAAACTAACCCTGAAATCAAAGTGGCCTAATG
AATGCTTATTTATTGCTCATATAAAGTCTGATATAAATCAGGTAGCATTCCTTCACAGTGACTCAGGGACCTAAATT
GCTTTCATCTTGTGGCTCTGCCATGTCAACATTGAAACTTCAGAGGTATTGACAAAGGAGAGCTGAGAAATTGACAC
TAACTATCAAGTACCTCAGGCAAGAAGTGATGCATCACTTCCTACACTTTCCCACTTTCCAGGATTCAATCACAATT
GCACTCACGAAACCTCAAGGGAACATGAGAAACATAATGAAATCAATACCAAGATATCCAAAGAGTACTAATTTTCT
TCACCACAGAAGGGATATTGGACAGACAAAAAATTTTGGGACAGAGGAATATCCACTAAATGATCCTTCCATGCCCT
GCTCAGATGCTACATTTTCCATTAAGGCTCTCTTGACCAACCCATAAGGAGATGATCTCCAACTCCTCTGAACATCT
GGAACATTGCATTCTACTTATTTTTACTGTCTCCTATTGTAATAATTGTTGGCTATTCCTTATTAGACAAATTATAA
GCTTTTATGGGACGGTGATCAAAAGTCCTAATTTATCTTGTCTTCCTACAGCATTCAAAGCATTGTAGACCTTCAAT
ACATAGCAGTCTTCGAAGCCAGAGTATTAGTATTTTTTCTAAAACTGTATTCATCGCAACGAACTCGATGGATGTTC
AATAAAAATGTATTGAATAGTCATTCATGCAGATAGCAAATTTCTTAAAACATTTCTTACTCAATGTTTACTTATAT
TCATCATGAATATAAATTTAATATAATAATATCAAATCCCATTACATTTAGTACAAAGTAAATATACCAATAATTTT
ATATATGTTTAAGTGAATTTTTATATTTCATATTATTTGTGAATGATACACATCGTCGTGGAAAATTTACAACGTTG
AAGTAAATTATACAATATAGGGTATTTGTATTAAAATACATTTCATAGAAGGTGCTTTAAATTTAAAAAGTTAGCAT
GAATACATATTAAATGTAAAAGAGTCTTTAAAATGAAAAATATTTCACACTTGTGGGTTTGATCTAAAAATATCAGT
CGCTATATGATATGGCTAGACAGATTTGAGTAGGTATAATTTTAATGAATAATATTGTGTGATATTTTAGGGTCCAA
AATTGAATGTTTCTATTAAGTAACAGATATTTAAATGCCAAGCAGAAGCTACACTGGCAAAACAAGATTTAGTATGT
TATCTTAAGTGTATCCTAAATCCTCTTTCATCTTATGGAGAAGAAATACATGCTTTTAGTTTCCAGCCACTCCTGTC
TATATTCATTTAAAATAAACTATTATTTATATCAGATATGATGCCATGTGAGTCCACAATTTGTAGTATAGAGAAAT
AATTTCATTGAATTATTTTGGCTGTTTGTACCTGAGACCTGATTCTGAATCATTTATTTTTCTATTTAAGTTGTGCT
ATTTTATGATCCTACCTATGGCTATGTTCAAAACAATGACAAAATATATTTTACCCTGATCTTTTTTTCATTATTAT
ACTTTAAGTTCTGGGACACACGTGCAGAACGGGCAGGTTTGTTGCATAGGTGTACACGTTCCATGGTGGTTTGCTGC
ACACATCAACCCATCATCTACATTAGGTATTTCTCCTAATGCTATCCCTCCCCTAACCCCCCATCCCCTGAGAGGCC
CCGGTGTGTGATGTTCCCCGCCCTGTGTCTATGTGTTCTCATCGTTCAACTCCCACTTATGGGTGAGAATATGTGGT
GTTTGGTTTTCTGTTCCTGTGTTAGTTTGCTGAGAATGATGGTTTCCAGCTTCATCCATGTCCGTGCAAAGGACATG
AACTCATTCTTTTTTATGGCTGCATAGTATTCCATGGTGTATATGTGCCAATTTTCTTTATCCAGTCTATCATTGAT
GGGCATTTGGGTTGGTTCCAAGTCTTTGCTATTGTAAATAGTGCTGCAATAAACATACGTGTGCATGTGTCTTTATA
GGAGAATGATTTATAATACTTTGGGTATATATGCAGTAATGGGATTGCTGGGTCAAATGGTATTTCTGGTTCTGTAT
CCTTGAGGAATCGCCACACTGTCTTCCACAATGGTTGAACTAGTTTACAGTCCCACCAACAGTGTAAAAGCGTTCCT
TTTTCTCCACATCCTCTCCAGCATCTGTTGTTGCCTGACTTTTGAATGTTCGCCATTCTAACTGGTGTGAGATGGTA
TCTCATTGTGGTTTTGATTTGCATTCCTCTAATGACCAGTGATAATGAGCTTTTTTTGATATGTTTGCTGGCTGCAT
AAATGTCTTGTTTTGAGAAGTGTCTGTTCATATCCTTAGCCCACTTTCTGATGGGTTTTTTTTTTTCTTGTAAATTT
GTTTAAGTTCCTTATAGATTCCGGGTATTAGCCCTTTGTCAGATGTATAGATTGCAAAAATTTTCTCCCAATCTGTA
GTTTGCCTGTTCACTCTCATGATAGTTTCTTTTGCAGTGTAGAAGCTCTTTAGTTTCATTAGATCCCATTTGTCAAT
TTTGGCTTTTGTTGCTGTTGCTTTTGGTGTTTTAGTCATGCAGTCTTTCCCCGTACTTGTGTCCTGAATGGTATTGC
CTAGGTTTTCTTCTAGGGTTTTGATGGTTTTCGGTTTTAGGTTTAAGTCTTTAATGCATCTTGAGTTGATTTTTGTA
TAAGGGGTAAGGAAGGGGTCCAGTTTCCGTTTTCTGCATATGACTAGCCAGTTTTCCCAACACCATTTATTAAATAG
GGAATCCTTTCCCCGTTGCTTGTTTTTGTCAGGTTTGTCAAAGATCAGATGGTTGTAGATGTGTGGCATTATTTTTC
TGGCCTCTGTTCTGTTCCATTGGTCTATATATCTGTTTTGCTACCAGTACCAGTCTGTTTTGCTTACTGTAGCCTTG
TAGTATAGTTTGAAGTCAGGTAGCATGATGCCTCCAGCTTTGTTCATTTTACTTAGGATTGTCTTGGCTATACAAGC
TCTTTTTTGGTTCCATCTGAAATGTAAAGTAATTTTTCCTAATTCTGTAAAGAAAGTCAGTGGTAGCCTGTTGGGGA
TAGCATTGAATCTGTAAATTAATTTGGACAGTATGGCCATTTTCACTCCACTGATTCTTCCTAACCACGAGCATGAA
ATGTTTTTTCCATTTGTTTGCGTCCTTTCGTATTTTCTTGAACAGTGGTTTGTAGTTCTCCTTGAAGAGGTCCTTCA
CATCCCTTGTAAGTTGTATTCCTAGGCACTTTATTCTCTTTGTAGCAATTGTGAATAGGAGTTCACCCACGATTTGG
CTCTCTGTTTGTCTATTCTTGGTGTATAGGAATGCTTGTGATCTTTTCACTTTGATTTTGTATTCTGAGACTTGGCT
GAAGTTGCTCGTAATGTTAGGGGCAGCCAGAGAGAAAGGCTGGGTTACCCACAAAGGGAAGCCCATCAGACTAACAG
TGGATCACTCTGCAGAAACCCTACAAGCTAGAAGAGAGTGGGTGCCAATATTCAACACTCTTCAATAAGGGAATTTT
CAACCCAGAATTTCATATCCAGCCAAACTAAGTTTCATAAGCAAAGGAGAAATAAAATCCTTTACAGACAAGCAAAT
GCTGAGTGATTTTGTCACCACCAGGCCTGGCTTACAAGAGCTCCTGAAGGAATCACTAAACATGGAAAGGAAAAACC
GGTACCAGCCACTGAGAGAAACATACCAAATTGTAAAGACCATCGACCCTATGAAGAAACTGCCTCAACTAACAGGC
AAAATAACTAACCAACATCATAATTACAGGATCACATTCACACATGACAATATTAACTTTAAATGTAAATGGGCTAA
ATGCCCCAATTAAAAGACACAGACTGGCAAATTGGATAAAGAGTCAGGACCATCAGTGTGGTGTATTCAGGAGACCC
ATCTCACATGCAAACACACACATAGGCTCAAAGCAAAGGGATGGCGGAATATTTTCCAAGCAAATGGAAAGCAAAAA
AATAAAAAATTAAAAAAAATAAAAAAAAAAACAGGGGTTACAATCCTAGTCTCTGATAAAACAGACTTTAAACCAAC
AAAGATCAAGAAAGACAAAGAAGGGTATTACATTACATAATGGTAAAGGGAGCAACGCAACAAGAAGAACTAACTAT
TCTAAATATATATGCACCCAATACAGGAGCACCCAGATTCGTAAAGCAAGTTCCTAGAGACCTACAAAGAGACTTAC
ACTCCTACACAGTAATAGTGAGAGACTTTTATACTCCACTGTCAATATTAGATTAACGAGACAGAAAATTACCCTGA
TCTTTAACATTCCAGGCAAGTATGTTTTCAGTCATACATAGTACGTGAATCTGTTATATTTTAAATCCAAGCTTTTG
GAGGACAAATGATTTCACAGTTATACAACTCAGCCTCTCCCAGACTCAAGTCTCTCACTTGATGCAGTATTCCCACC
CCATCTTCAATAAAAGAAGAAAGCATGAAACATCGCATTGATATAGGGAAAGTATATCTGAGTTTTCTATATAGCAT
ATAAAGAAGTAAAATAACTCTGTGATTTGTGATGAGATAGATTTAGCTGCAACAATGAGGTCTGGATATTAATATTG
GATTAACTTCCTTTGAGATTGCAACTTCCATGTGGCATTCAGCACTTTCCTTGTTTTCTCATATGTAGGCCGAGAAT
AAAATTATCCTGTTTCTATTTCCCAACATGAAAGGTCTTTTAACTGTTTGAAGACATATGTTTCATGTGAAACCAAG
GAATTGCTACTGCAATTTTGTAAGACAGAAGTTAGCAAACTTATTCTGTAAGGAACAAGATAGTAAATATTATAGGC
TTTGCGGGCCATATGGTCTATCTCACAACTATTAAACTCTGCCATTTTTTTTTTTTTTTGCAAAAGCAACCATAGAC
AATATGTAAATGAATGAGTGTGATTATATTCCAATAAAACTTTATTTTTAAACTACAAGACTAGTTCTTAAAATAAA
ATTTAACAAAGAACTATTATATGATCCAATAATTCCACTTCTGGGTATATACCCAAAATAATTGAAAACAGGTATTC
ACACAGATATTTGTACACCCAAGTTCATAGCAGCATATTTTACTGTAGAGAAAAGGTAGAAACAAATGTACACTGTG
TACACTGACCAAGAATGGAGAAACAAAACGTGGTGTATCCATACAGCAGAACAGTATTCTGCTTTAAAAAGGAATGA
AATTCTGTCACATCCTAAAATAGGGATGAACTTTGGAGATATTCCACCTAGAGTAGTTAAATTCTTAGACAAATAGT
TGCATGGTTGCCAGGGACCAGGGAGGGGTAGGGGAAATGAGCAGTTCTTGTGTCATTGGTATAGAGTTTCAGTTTAG
AAAGATGAAGAGTTTTGGAGATAGATGGTGGTGATGTTTACATATCCATGTGAATGTATCTAATGCCACTGAACTGT
ATACACTTACAATGGTTGAAATATTTTGTATATTTTCCCACAGTTAAAAAAGACAAGCCCATGTGCCCAAATTGCTG
ATATAAATCTGTATTCCTATTAGTGAGCAGTAATGAGCCATGTTTAGTGCAGCTGATATGGTTAAGATGGAACAATT
GCATGTAATGCAATGGTGGGTAACATCTAATAAAAATTGGTACTCACCAATTTCCATTTCCATTGTGGATATAAACA
CCCTTCACATTATGAATCATGTTATTTGTTATTTGTGTACTTACTTATTAAATCACTTTTCTCCATCTCTCTCACCT
CTATGACTCTGTACATGTACAAACAACATACACACTGTGAACTTCTTTAGAATGACAGTTACATTTTCGTTACTTGA
TTCATTGTTTGGCCAAGTAGATACCCAGTAAGCATAACTAGCAAGTGAATAATGTATATGTGATCATTTTCCTTTTC
AAAAACCACTTCTTATCTCTACCACACCACCACACATTATTATATGCTACACTTCTTATATAAAACAAAAAAGAATA
GGCCAGGTGTGGTGGCTCTCGCCTGTAATCCCAGCAATTTGAGAGGCCGAGGTGGGCAGATCACTTGAGGCCAGGAG
TTAGAGTCCAGCCCAGCCAACATGGTGAAACCCCATCTCTGCTAAAAATACAAAAATTGGCCAGGCGTGTTGGCGCA
TGCTTGTAATCCCAGCTACTTGGGAGGCTGAGGCAGGAGAATCACTGGAACCCAGGAGGCGGAGGTTGCAGTGAGCT
AAGATCATGCCACTGCCCTCCAGCCTGGGCAACAGAGCAAGACTCTGTCTCAAAAACAAACAAAGAATAAATAAGCT
TTAATAAACTCTTTTGCAGAAGAGTTTTCATTTTTAAACATAGAGGAAAAAGAAGATGAGGATGAACGATAGTGATG
CCCAGAAGAGAAAATTGTGTAAGACAAAATCAATTGACCAGGCGTGGTGGCTCACGCCTGTAATCGCAGCACTTTGG
GAGGCTGAGACGGGCAGATTATGAGGTCAGGAGTTCGAAAACAGCCTAGCCAATATGGTGAAACCCTGTCTCTACTA
AAAATACAAAAATTAGCCGGGCTTGGTGGTGCACGCCTATAGTCCCAGCTACTTGGGAGGCTGAGGCAGAAGAATCT
CCTGAACCCAGCAGGCGGAGGTTGCAGTGAGCCAAGATCACGCCACTGCACTCCAGCCTGGGTGACAGAGCGAGACT
ACATCTCAAAAAAAAAAAAAAAAGATTAAATCAATTTTAGATTGATATAGAGATAAATAATTCCCTAAGTACAATTA
AAGCAAGGATCAGACCCTGATGAGTAAGGATGATCTAACAAAGGGCTGACAATATAAAGTGAATGCGATTGTCACCA
AATAAACATCTCCCAAGAGCTTTGGGACAATCTTCAGCTAAGCATATTCTGCACTTTACATCGTTCTGCCATATGCC
CTATGTAGCTTTTCATCCTCCAGAAATTTGAATAGCACACAGTATTTTTTGGCACCGAGTAATTGTGGAACTTAAAC
TTCCTACAAAGAGAGAAGCTGTTTCAGTCAGCTAAGTGACCAGTTAGTAAGCAACTTCTATTCTCTTAGTGTCGATA
GGGTTAAAGACAGTCATAAGCTCCCCAAACCCTTGGCCGCCTCAATTTTTTCATTTAAATACTTATCTTCTTATCTT
CATCTATTTTTTAGAGTCACACATAAACTCTGTCTATGAGGTTCTTAAAGACCTGGACAATATATTTAATCTCTGTA
TCCTTAAATCTCACCATGATATCTCAAAGATGGTAGATAAAACTGTTTTCCCTCACTTCTCTGACTCCATACCAATA
CTTAATCGATCGGGAAAGTTTTATAAACCATTTACCGTTGAATATAAATATATATACACACACAAACATATATATAT
TTATATATATTTATGTATATATATATTTATATATCTATATATATTTATGTATATATTTATATATATTTATATATATT
TTTATATATTTATATATTTATATATATTTATATATTTATATATATTTATATATATTTATATACTTATATATATTTAT
ATATATATTTATATATATTTATATATATTTATATATATTTATATATTTATATATATTTATATATATATTTATATATT
TATATATATTTATATATTTATATATATTTATATATATATTTATATATTTATATATATATATTTATATATTTGTATTT
ATATATATTTATATATACACATATTTATATATTTATTTATATATGTTTATATATATATATATATGGCACTATATATC
AGTGAGCAAAGGCATCATAATCTTTATTTTTATTTCTGTATTCATTTATTCAACATATAGTAATTCTGATCCTGTCT
CAAAAACTCTACAGAAATTAACAAAGAACACATTGTTTCTGCCTTTTTGGAGCTGAATGTTTAGTGGGGGAAAATGT
ATTTTAAGGCAATAATCATATACATGAGTGTATATTTACAAGCCGCAGTAGGTGCTATGAATGAAAAGTATAAGGAG
CTATGAGATATTCTAACAAAAAGAAATGCTGCAGTCTAGAGACTGAGAGTAATTATCTGTGAGGAAAAGTATTGAAG
CAGAGACCTGCAACTCAGTAAAGAGTGTAGGGAGTCACATGAACAAAGTCTTGAGATGTTGGTAGAGATCTTGGAAT
GTTCGAAATTCTAAAAGAAAGCCCTTGTGTATGGGGTATGGTGAGAGAGGGAGAGCGTGGCAAGTTTTACGTTGTAC
AAGTCATTGTAGGCCACAAAAATAAGTATAGATTTTATCTAAGTGCAACGGAAAACCATTACAAGTTTATTTTTTGA
TTCTTAAATTTTGTTTTACTTATTTATTTATTTCAAGACACAATCTCACTCTGTTGCCCAGGCTGGAGTGCAGTGAC
ATGATCACGGCTCACTGCAGCCTTGAACTCCTTGGGCTCAAGCAATCCTCCCATATCACCCTCCTACCTCAGCTTCC
TGGGTAGCTGGGACTACTACAGGCATTTGCCACTATGCCTGGCTAACTTTAAACAATTATTTTGAAGAGACAGGGTC
TTGTTACGTTTCTAAGTTTCTAAGGCTGATCTTGGATTACTGGGCCCAAGCAATCCTCCCGCCTCATCCTCTTAAAA
TGGTGGGATTACAGATATGAGCCACCTCACCCAGCAAGGTAGTGACATAATCATTGTTTTTGCTGAAAACAATGTAC
TTTAGGGAACCTCCCTGAAAACAGAATGGCCACTTAGAATACCAAAGCATAGACCAAGCCTTGAGATGGCAATGGAA
GTGGAAATAAACATGTCTTTGATACTTATGTTAATGGAGCTATTAGTAGCAACTTTTGACAGATTTGAAAGTGGAGT
TGAAAGACAGTGAAGTGTCAGGGATGATCCCTGAGCATATTAGATAATATTGAGCTGGAGAACAGAAAAGAAGGGGC
AGATTTGGAGGTGGAGACCTTTCTAGATTTATATTCAATTTGAGATACATCTGAAACATCTACATGAAACGTGGATG
AAATATCTGTGACCAAGATACATACGTGGGATACCTAGGCATGTAAATAATATTTAAAGTTATGTGAGTGGGTGCAA
TAACCTAAAGAGAGACTGTAGGTAGAAGATTGATCAGGACAAATCTTTGAGGAACTTTTTTTTTTAATTATTATTAT
ACTTTACGTTCTGGGATACAGGTGCAGAACGTGCAGGTTTGTTACATAGGTATACACGTGCTATGGTGGTTTGCTGC
ACCCATCAACCCGTCATCTACATTAGGTATTTCTCCTAATGCTATCCCTACCCTAGCCCCCCACCCCCAACAGGCAC
CAGTGTGTGTTGTTCCCCTCCCTGTGTCCATGTGTTCTCACTGTTCCACTCCCACTTACGAGTGAGTCCATGCAGTG
TTTCATTTTCTGTTCCTGTGTTAGTTTGCTAAGAATGATGGTTTCCAGCTTCATCCATGTCCCTGCAAAGGACATGA
AGTCATCCTGTTTTATGAGGTACTTTTAATGCATTTAGTGACTGGGTAAGGAATAACCCTGAAAAGGAGCACAAAAA
CCATCGAGGTATGAAGAAAGCCAGGAGATGTAGTTTCACAAAGCAGCCAGTGGACAGTAATGGACACCAGCATAAGC
TTAAGACAGCAGTGATAGTGTTCATCAGTGCTGAAAACTCATTGGAAGTGGCTGACCATATGTTGATTGCGAATGCA
ATGCCTTCTCCCATCTCCTATTGCTTTTCACTGTTTCCGTCATGTCCAGAGTGAAAACACTGGGGAGGCAGAACTGT
GTACCAAGAAAAATGTTTTGTGTCAGAGGAAACAAGAGCTCATTAATTTCCTTTTGACCCGCATGGATAGGAATACG
ATAAGCAGGAGAAAAATAAAACCTGCTAAACTAGGCAAATAAAGCTTAATAGAAAAGAGAGAAAATATTGCTTACAA
AGGGAAGAAGACATTTGCCACAATATTAGCTAAAAAGAGAGTGAGGAAAATCCACATTTACCATTTTTATTATGTGT
TTCCAAAGTCGACATTAAGTGAATATTATCACTTTGAAGACAGAGAATAGATTGTAAGCAGAAGAGGAAGTGGGATT
TTTTAAATGGCATATATTTAAATGTTGAAAAACCCATTTCCAATACTCCCACTGAGATATATGCAATAAAAACACTC
CGCAATCAGTAATATACATGTGAAGCTCCTAAAAATCCTCCATTGAGAAGATTCTATAAAACTATAAAGTATTATTA
TTTCTGCTATCAGAAAATAAACACATACTTTCTAAGAACAAGATGAAGACCTTAATTCATCTTCCCAGGAATATACT
TGTGTACTTAATGGTTCAAGGCGTTTGTGATGCAAAAAAAGTGGGACATTATGTACAAAATAGTTTTAATCAGAAAA
TGAAGGAAGTTTGGTATGGTATGGTTCTAGGGTTGCAAGTTTTCCTGCAAAAAGGGAGTACTTATGGCTAGGTGATG
CAGCCAAGGAAGCATCTCTGAGGACAGGTTATCCTGGCTCCTATTCCAAAATATTTAGGTCTCCTTGAGTGTTCGAT
CCTTGAACCAAGGACAAGAATAGAAAGAAATAAAAGCCACTTGTATAAAGGTTGCCTTCATTTTGTGTCGAATATTG
ACAGGAGAAAGCCAGATTTGTAGCTTTTCCAGGGATAAGTGAGTCTCAGACACAGTTAAGAGCTTGGCTTTGGAATT
AGATTGACCTGAGTTTGAATCGTGGCTCCATCAATCTCTGATTGTGTGAATTTTTGGCAAGTTTTAGCTGAACCTCA
ATTTTCTCATAAGTAAAATAGGCATAATACAGCTATGATTCTGACCCTTGCCATTAGCACTCTGAGCTACTTGTCAG
TGTATCCTGGTCTCTTCCTCAAGCACTTTTCCACTTTGTTATTTCAAGATTCTTTGAAATTATGAACAGTAGCTCTT
GATCACCAAACAAATGGGAGACCATAGATTCAAAGGAACATTTTAGTAGGATATTTGATAAGCTTTGTATTTTTTTG
TGCTTTGAAAATTAGAGTAGATTTAGATTTAAGGAACTATAAAGCTATATGTTTACAAAACTTTATTCAGTTGGGCA
GTTCTGACTCAGTAACTGAAATAAAATTGATGAAAACAAAACATATGTTTTACTGTTGTGTTCACATTTAAAGATTA
TTTAATGCATAACCAAAATAGGTTCAGCAAATGTTTATACATACATGTACTTAGATATTTGTAATTCTTATGGTAGA
GCATGGGAATGAATGAGAATTCTCCTAGGCTGGAATAAAACTAATGTCTAACTAAGAATTGGCCTTTCACTTCACGA
ATATATATCATTATATATAAAAGATTCATCCTTTGTTAACTTATTTGTTTAGCTTAGTACTTTCTAAGTACTTTTCT
ATCCTGTCTTGGTCCCTATATAAGAATGATACTATTATAAAAATGAACATCCTATTATTTCATAATGATTAGCATAT
CAGCATATTTAAGAGCAATGCCTATATCTTTACCTTCATTATTATTTTCATGTTTAAAATCCTGATAATTCTAGAAA
AACAAGGTTGTAATTATCCATTCATAATAAAAGTCACACCAAGTTATTCTTGATAATTAACTTGGCATAGTGGAGTG
CACTGAGCAATTAATAATTAATTACGTGGTTACTAGCTCAAATTTTATTGAGAACCAAGGAGTTAAAATTAATAGTA
TTGTGTTTGCAATGAGTTAATTACTTTGTTTCTTTGTTTTTTTTTTTGGAATAGTGCCATAATTAGTATCCTAATAG
AAACAGAATATAAAGTGGGTTTCTGAGTGCTTGCTTGTCGAGGTGTGTAACAACATATATTTTAATTATAATTTGAG
TCACTAATTGTTGTCAAATACAATTTTAATTTCTTAGTTCAGAGATTAAAAGATATGTAAACAGCTCCAAAAAAGAA
TATGATAAGATGGTTTATACAAGTAAATAAATAACTATATGACTTCAGAAGTTTTATTTAACAACAGTTGTATAAGT
GTTTTAACTAATGTTAACAATGCCTTTTTTGTAAATCTCCTCTACCACAAATATGAGATAAATTTAAGAACGGCATT
TTCATTAAATATTGATTATTAAAATTCATATATAAGTATTGGCTGTACCTGAGCATTTACAGTGAAACCATGAAGGA
CTGGCAGGTTTGGGGCTGTTTGATAATATCCTTCCTTAATCTTTCTTCGTAGCATCATTGTTTAATTTTGGCTGACC
ACAATCACTTATGAAAAGAAACAGCTCAAATAATTAGTATAACCTCTTTCTCTTCATAAAACCTTCAAATCAATGTG
GTCATAATTATGAAAGTGTAAGATTATAAAATCTTCTCATTTAAATATACATCTTCATATTAGCAGACTTGGCTAAT
TTCTTCATTTATCCTTGACATCCACACTTAAACTCTGTCAAACTGGTCATTCATAGACTCATTATTCATTCAACAAA
GATTAGTGTGACCCTACTATGATTCTGAGGGCTAGAAATATAAAACTGAATAACATATAAACCCTGATTTCTGTATT
TATATATACAGAAAGGAATTATGAACTCACAGTTTTAATGCACAGTGACGAGTCCTCAGTTCATGTCTGCCTTGGGA
GCTAGGCATATACCAAAGCAGGACTGTCTAACACAGTTTGGGGGTCTCAGTCTGGAAGGATCCGGGTGAGATGATCC
TTGAATTGAGTTCTAAAAAATGAGGAGAAATTATCCAGATAATGGAAAGTTTAGGAGAAATTAACCAGATAATGGAA
AGTTTAGGAAAGAAACACTATTTATATGTCAAGGACAAAGTTTGACCAAATGTGTCTCTGAATCTAACTGGCTTTAT
GTAAGATAAAATTAAAAAATAATTGTCACTAAGGTTTTTCACATCTGCAGCAACTACCATCATATCTGGCTCAATAT
TTAAAAGAAATACTTAACGATTTTTGGCATTTATTGCTACTATAATAGATTGTGCTATTTAAAGAGAAATTCTAGAT
ACATGCCAAAGTATTTTCAGGTGAAGTAATGTGATTCGTGGGTTTTGTTTTGACAAACTTCCAAGGAAATTATTTTT
AATATAAAGAGCAATGGAGGAAGATTAAACAAGATTCGCAAATGTTGATAATTGCTCAAGGTGGTGACAGGTGCATG
GGGGTCCATAATATACTGTTCTCTTTACTTATATATGTCTTTGAAGATTTTCATGATGTAAAATTTTAAAATCAGAA
TTACAGTAATACCTCTTTCTTTGACAATCGTTTTCAAACTTGGTTCAACAACTGAATTTAAATTACCTACATGGCTA
TAAAAATAAATTAGATATCCTGGTTATTTGGTCTGGGGTGGGTCCTGGCCATATCTGTAGGGTGTGTGGGTGTGTGT
GCATTTAGCTTCACATTTGACTCTGAAGTACGTGGAAAACCAATTCTGGGCTCTACATATGCAATTCCAATGCCTGA
CATTAGGTGGTGCTGGTTGTGAGCCATGATTTCAGGTTTGACTTGTAGAGTTAACAGAAGTGATATCACCACCGATC
CCACAGAAATACAAACTACCGTCAGAGAATACTACAAACACCTCTACACAAATAAACTAGAAAATCTAGAAGAAATG
GATACATTCCTCGACACATACACTCTCCCAAGACTAAACCAGGAAGAAGTTGAATCTCTGAATAGACCAATAACAGG
AGCTGAAATTGTGGCAATAATCAATAGCTTACCAACCAAAAAGAGTCCAGGACCAGATGGATTCACAGCCGAATTCT
ACCAGAGGTACAAGGAGGAACTGGTACCATTCCTTCTGAAACTATTCCAATCAATAGAAAAAGAGGGAATCCTCCCT
AACTCATTTTATGAGGCCAGCATCATTCTGATACCAAAGCCGGGCAGAGACACAACCAAAAAAGAGAATTTTAGACC
AATATCCTTGATGAACATTGATGCAAAAATCCTCAATAAAATACTGGCATACCGAATCCAGCAGCACATCAAAAAGC
TTATCCACCATGATCAAGTGGGCTTCATCCTTGGGATGCAAGGCTGGTTCAATATACGCAAATCAATAAATGTAATC
CAGCATATAAACAGAGCCAAAGACAAAAACCACATGATTATCTCAATAGATGCAGAAAAAGCCTTTGACAAAATTCA
ACAACCCTTCATGCTAAAAACTCTCAATAAATTAGGTATTGATGGGACATATCTCAAAATAATAAGAGCTATCTATG
ACAAACCCACAGCCAATATCATACTGAATGGGCAAAAACTGGAAGCATTCCCTTTGAAAACTGGCACAAGACAGGGA
TGCCCTCTCTCACCGCTCCTATTCAACATAGTGTTGGAAGTTCTGGCCAGGGCAATCAGGCAGGAGAAGGAAATAAA
AGGTATTCAGTTAGGAAAAGAGGAAGTCAAATTGTCCCTGTTTGCAGACGACATGATTGTTTATCTAGAAAACCCCA
TCGTCTCAGCCCAAAATCTCCTTAAGCTGATAAGCAACTTCAGCAAAGTCTCAGGATACAAAATCAATGTACAGAAA
TCACAAGCATTCTTATACACCAACAACAGACAAACAGAGAGCCAAATCATGAGTGAACTCCCATTCACAATTGCTTC
AAAGAGAATAAAATACCTAGGAATCCAACTTACAAGGGATGTGAAGGACCTCTTCAAGGAGAACTACAAACCACTGC
TCAAGGAAATAAAAGAGGATACAAACAAATGGAAGAACATTCCATGCTCATGGGTAGGAAGAATCAATATCGTGAAA
ATGGCCATACTGCCCAAGGTGATTTACAGATTCAATGCCATCCCCATCAAGCTACCAATGCCTTTCTTCACAGAATT
GGAAAAAACTACTTTAAAGTTCATATGGAACCAAAAAAGAGCCCGCATCGCCAAGTCAATCCTAAGCCAAAAGAACA
AAGCTGGAGGCATCACACTAGCTGACTTCAAACTATACTACAAGGCTACAGTAACCAAAACAGCATGGTACTGGTAC
CAAAACAGAGATATAGATCAATGGAACAGAACAGAGCCCTCAGAAATAACGCCACATACCTACAACTATCTGATCTT
TGACAAACCTGAGGAAAACAAGCAATGGGGAAAGGATTCCCTATTTAATAAATGGTGCTGGGAAGACTGGCTAGCCA
TATGTAGAAAGCTGAAACTGGATCCCTTCCTTACACCTTATACAAAAATCAATTCAAGATGGATTAAAGATTTAAAC
ATTAGACCTAAAACCATAAAAACCCTAGAAGAAAACCTAGGCATTACCATTCAGGACATAGGCATGGGCAAGGACTT
CATGTCCAAAACACCAAAAGCAATGGCAACAAAAGCCAAAATTGACAAATGGGATCTAATTAAACTAAAGAGCTTCT
GCACAGCAAAAGAAACTACCATCAGAGTGAACAGGGAACCTACAACATGGGAGAAAATTTTCGCAACCTACTCATCT
GACAAAGGGCTAATATCCAGAATCTACAATGAACTCAAACAAATTTACAAGAAAAAAACAAACAACCCCATCAAAAA
GTGAGCGAAGGACATGAACAGACACTTCTCAAAAGAAGACATTTATGCAGCCAAAAAACACATGAAAAAATCCTCAC
CATCACTGGCCATCAGAGTAATGCAAATCAAAACCACTATGAGATATCATCTCACACCAGTTAGAATGGCAATCATT
CAAAAGTCAGGAAACAACAGGTGCTGGAGAGGATGTGGAGAAATAGGAACACTTTTACACTGTTGGTGGGACTGTAA
ACTAGTTCAACCATTGTGGAAGTCAGTGTGGCGATTCCTCAGGGATCTAGAACTAGAAATACCATTTGACCCAGCCA
TCCCATTACTGGGTATATACCCAAAGGACTATAAATCATGCTGCTATAAAGACACATGCACACGTATGTTTATTGCA
GCACTATTCACAATAACAAAGACTTGGAACCAACCCAAATGTCCAACAATGATAGACTGGATTAAGAAAATGTGGCA
CATATACACCATGGAATACTATGCAGCCAGAAAAAATGATGAGTTCATGTCCTTTGTAGGGACATGGATGAAACTGG
AAACCGTCATTCTCAGTAAACTATCGCAAGAACAAAAAACCAAACACTGCATATTCTCACTCATAGGTGGGGTGGGA
ATTGAACAATGAGATCACATGGACACAGGAAGGGGAATATCACACTCTGGGGACTGTGGTGGGGTGGGGGGAGGGGG
GAGGGATAGCATTGGGAGATATACCTAATGCTAGATGACGAGTTAGTGGGTGCAGCGCACCAGCATGGCACATGTAT
ACATATGTAACTAACCTACACAATGTGCACATGTACCCTAAAACTTAAAGTATAATAAAAAAAAAAAAGTTCGTTCC
TCTTGTTAGAATTTTCATACAAATGCAAGACTCCTTATTTTCAAAGAGAAAACAAAATTTTATATAAACTAATACCT
GAGAAGAATAGGAAAAATTAATGATGTATGGGATAGGTACTGATAAGAACCTCTTCTTTTGTGTACATTGTGATAAA
CTAATGTTTAACTTGAGTCGATGTGCTGGAAATACTTGTTAAATTTTCAAGAATTTTGAGATCTGTTAAACACAACC
ATTATCAAAAATTAAATTATGGTAACTTACATTAAATGAAATATATTGTTCACAAATGTACTAGATGCACAAAAGTC
ATCACTACCTAATTCTTTTATTATATTTCACTATTATCTACGGGTTTGTTACCTGTATAATGGAATGACTGTATAAT
GGTGTAGTGCTAACTTCATGTTTGGTGACATTATAATGATAGCTTAAAATTAAACATACTGGGAATATTCAAATATG
TCCTTCCTCTCAGGGGATCTGATTGTTAGACATTTACCAGCATAGCACTGTCTTAATTAACATCATATAATTGTCTT
TTATCCTTAAGATGTTGAGCCAGTAAATATCACAGGAGAGTTTGTTCCTTAGGTTTTAATACTTGCTTTACCAGTTT
ACTGTAGCATCATGTGAAACCAGCTTCTAGACCAGCTGTCTTTAATTGGTATGTACCCAAGCTAATCCTTGTCAGGC
TATCATCAACCCAAGCACTTTTGAAGTTATTTTTATTATTACGCTACAGAAAGATTTTATGGCCTTTTTGCTCTTTT
CTTTACCCTAGCCCTAGGATCATATAATTTCATCTCCTATTTATTCCTATTTATTTCCTTATCTAAAATCTTACTTA
ACTAGCTTGCTTGCCTGCCTGTCTGCCTGTCTGCCTGCTTGCTTGGTTTTTCTTTTTTTCTTTCTTTTCTTTACTTC
TGCTTTCCTTTCCTTTCTTTTGTTTCTTCCTTCCTTCCTTCCTTCTTCCTTTCCTTTCCTTCTGTTCCTTTTCCTTT
TCCTTTTCCTTTGCGTTCCTTTGCTTTCCTTGCTTTCTTCGCTTGCTTTCTTTTCAGTACCACAAAAAAACTAACAA
TAACAAAAATATTGCTACAAAAATTGTGACCTCCCAATTCTATCCTCATTTCACGGACCCATAAAATGATTATATAT
CTATGCCAATGCGTTCTAACTACATGATATTATTATTGATGATAAGAGAATCCAACCCTCAAGAAATGAAGTATTAT
AATGTTTTAGAACACACAGAAGCATTTTTAACCAAAATGTTCCATGTGATTTTATAGGGTGCATGTTTTACCTTTTC
TTATTTGGAAAAGTAGCAAAAGTATTAACAAATAAAATAAGTTACATACTCTTTGTATATAAATCTTTCGTGTACAT
AATAATGCCATTTCCCTTCTAGTGCTTCCATTATTGAAAAAAAATAGACATATAGCAACGTGCTAGACATTGAAGTT
AACTGTTAAAAAGTCATCAGAGGGTAATGTTAAAGGTGGTATTTCAGGTAATAAAGCATATGCAGAATGATTCTATT
GGCTTTATTTCCAACATGAGTGTTATTAGTACCAAGGACACTGAAGTTTAAAGGAGGGGAGTGATGAGACTGATCAT
TACTTTGTCTTCTGTAACAGGACTTAGGTGGCCCAGAGTGATCCTTGTTGGTTGATTGGTTAGTTGAACTTCTATTT
TTGTTACTAGGCCTGCAGATCTGTGGAATCTCCAGAGTGCTGTGTAATGTTACATAGGCCACACACCAGACACTGTT
CCCTGAAGGACTGAAGGTGGAATTGGAACAATATGTAGACAGTTCTTGTTGCAGTTACCAAGGAAATTTGGTTAGGA
TTCAGGCCTCCATGTCAGAAAAGGAAGTCTATTTTATGTGCTAGAAAAATAACCCTAATCATCTTATCTTGGGTCTC
TCTGGGGAGCATGATGATCTACCTGGTCTTGTAAACTGTATAGAACCGGCTGGGTTGAGACACATATGTCTCATAAC
AGAGCTTCCACTTAAGGCTATAAAATAGTGGTCCCTCTATTGGGTATATGCCTTATGGAAAAATATACGTTATGCTG
GTACCTATTATAAAAACCAATGCAAAATGATTAGAGATTTCTAATCCAAGTGTTATCCTCATCTGCTCCCAAAATAC
AGCAATGAAAAAATAGAAAATAAAAAAGGATATATTTACAGAACTGTATTTACAGAAATATATGTTGATGACAAGAT
AATCAAATACATATCAAATCATGATGTGTATTTACAGAAATATATTTACAGAAATGTAAAGCAGAGAATGGGGCGGA
AGTCCCTGACAGACTTGACACTGAACCACAAAACAACAGTGGAAGCCAGGAATTTGGATGCTGCGGGATAGTTGGGA
TTAAAATTGCTTCAAGCAAGGTGTGGAACTGGAGACAAACTCAGTATTCAAAACCAGAGGCTGGGCTGTGGTGCTTG
CATTCGTGAAAGAAAACCTGGGGAAAAAGTGCTTCTTCAGGCTGTAGCCTGACGCTATGGCTTGCCTAAACTTATGG
GCCTATGTAGGTGTAAGAATAAAGACATAGCCTTGCTGCTGGGAACTGTGTTGCATCTGTTCTCTTGCTTGATGAAT
GGATTGAAATTATCCCCAGTAACATTGCAGTGCAAGAACTTTAAACCTTAAAGGGGTGGCTGGAGACCACGGGACGC
GATGCACATGCAACTACTAAATGCTAGATGAGGATACCTGAGGAGAGAGAACAAGACACAAACGTGAACATGGACCT
CCAATAGAAATGACAATCGAAAACATACATTTATCTTCTCTCTCTCCCAAATTGTTCAAAGATTACGCTAAGGGAAT
AAAAACAATACAGTCATAAAATGACAAATAAAATGGAAGAAGAGACATCTGCACAGACATATCAAGTCAATGAGATG
AAAATTAGGACAAGTAAGCCAATTTGTTTTTCAGGATCCCAGAAAAACTTGTGGATTGAAGACGCTAGGTGTTTTAG
AAGGCCATATATTCCCGAGAGGGGCTGGAAATAAAGGGGTTGTTTTGAATCCCTTAAAAGGGGCAATTACATTTCAT
GTCCCCAGCTCAGGGTAGTTAATTAGACACTACTCCTCTCTCATCCCAGCAGACGTGAGTGAGTGGTTTGCTTTCTG
GTGAGGCTGAAACAACTTTTGGCATTTGGACAACAGGATTAGTTGATGGAAGAGAGAGCCACCATACTGAAAATACA
GGATTAAGTTAAAGTCAACATATTTAATGGTGAGACCAGCAGTCCCTCTTCACCTTCTTGGCTCTTAAGTTTCTATC
CTCTGGGCAGTAGATTATAAATTTATTCTCCTGAGAGCCTACCTGCAATACGAAAACTATTTTTAGTCAAGTTTTAT
TGCTATAACAAAATACCATAGACTGGATGCCTTATAAACAACAGACCTTTATTTCTCACCGTTCTGGAGGCTGGGAA
GTTCAAGATCAAGGCACTGGCAGGTTTGGTGTCTGGTGAAAGCTCATTTCCTGTCTTCTCACTGTAACCTCACAAGG
CAGCAGGTCCAAGGGAACTGTCTTGGGCCTCTTTTGTAAGGGTACTATTCTCATTCTGGATGGCTTTGGCCTTATGA
CCTAATTACCTTCCAAAGGCCCCACTTCCTAATGTCATCACCTTGAGGGTGAGGATTTCAACGTATAAATTTTGCAA
GAACACACTCAGATCATAGTAACCTACAATTAATGATATGTAATGATCCACCAGTGAAATAGCCAGATCTGTGCCTA
ATGACCATATATTGAAGCCCACCAAGTATAAGCCCTACATACGCACAAGGAATTTCCAAAAGCATTTAATGCCTCAG
TTTTAAGAGACAATCAAGGATTTCCAGATATTTGAACATGTCTCAAAGATGGAAGACAGAAACCAACATAAAAACAA
AAAGCAGAAATATTGGGAAAAAAACAATAGCCACATAAATGAAACAGAAATCTCTGCAGAAAGAGAGACAAGGCATA
GACCTTAAGAAAACTTTTTAAAGATAGCTATAAAATTATTATCTCACAGTGAGACTAGTCACTGTATTCATAAGAAA
GAACAAGAGACTATAGAAGGTGGTATTTCCGAGAAAGATAAGGGTTATTTAAATTAATAGTATAGCAGGTTAAGTTT
TTAAATTCTTAATTGTATAAATTTATGGGTTACCAGTGTAATTTTGTTATATGAATATATTTTATAGTGGTGAAGGC
AGAGCTTTTAGTATATCCACCACTGGAATAACATACATGGTATGCATAGCAGTTTTAGAGCAATGAAACTACTCTGT
GTGATACTATAATGGTGGATACCTTTCATTATATATTTGTCCAAACCCATAGAAGATACAACATCACAAGCAAACTC
TAATGTAAACTATGGACTTCGAGTGTTAATGATATATCAATGTATGTAAGTCAGTTATAACAAATGTACCACTCTGG
TGTGGGATGTTGATAGCGGGGTAGGCTGTGTGTAATGAGGAAGGGGGCCAGAGAGTATACGGGAAATCTCTGTACTT
TCCCCTCGATTTTGCTATAAATGTAAAATTGCTTTAAAAAATCAAGTCTATTAAAAATGAAGAGCAGAAATAAAAGT
TCAAAAGTAGAAGATAAATTTGAAGAAATATCTCATAAAGCAGTATAAATAGCCAAAAGCATGGAAAATGGGAGAGT
ATAAGAATCTTAGAAGATCATTCTAGGTAAGTGTTTCACAAATAGAGGTGATTTTGTCCTGCAGAGGACGTTTGACA
ATGTACGGAGACCTTTTTCGTTGTCACAAATAAGGAAAGGATGTACTACTGGCCTCTTGTGAAGAGTGACGCAAGAG
GAATGCACCATGCAGCCATCCACAACCAAGAATTACTCATTTCAAAGTGTCAATAGTGTTTATGTTTAGAAACCTTG
TTCTATGTAATCCAACATTCAAATAATAGATGTTAAAAATAAAACAGAAGAGAGGCAACTGTGGAGAGAAAATTATC
AAAGAAGTGACAACATCAGTTTTCTTAGAACTGAAGAAAATGAGTGTCCAGGTTTAAAGGGCTTACTAAATGCCCAG
CACAGCAAATGAGCTAAAAATCTACACCAAGGCTCATCATCATAAATTTTTACAAAAACAAGGACAAAGAGAAGTTC
ACAGATCAGAAATCAGAATGGCTTTGGACTTTTCAGCAATAGCCATGGAAAATAGCAGAAAGGAACACTTCCTTTAA
ACTTCTAGGAATAATTAACTTTGGCTTTGATTTCATATCCTATATGAAATTATGTTCACAAATTCTGTGATGCTCTT
CTATTCAAGAGTTACAGCTAAATGTCCCTGTCATTGAGTGTGGACTAGATTCAGTGACTCTTTTGTGATGATATGTC
ATTTCCAAGTGTAGGTTGTAGAAAGACTGTGGTTTATGTCTTGTGTTCTCCATCCACGCCTATCTCTCTCTGTGTCT
CTCTTTCTCTCTCTCTCTCTCTCTCTCTCTCTCTCATGCATTGCTTCTTCTGTGGGAACACATGTCATGAGCAGCCC
TATGGAGATGCACGTATGCTGAGAAACTAAGGCCTCCTGCAAAGAGCCACAAGAATGAGCTTAGAAGTGCATCATTT
AACACCAGACAAATCTCGAGAGGCTGCAAGCATAGACAACAGCTTAACTACAACCTCATAAAATAAGCTATGCCAGT
CCCACCCTGCTAAGCCACTCTCAGATTCTTGACCTTCAGAAACTGCCAAATAATAAACATTTTTGTTTTTAAGCTGC
TAAATTTTGGAATGACTTGTTATTCAACAATATATAAATTGTACACCCTGATAAACTATAAATTGTGCGGAGAAAAT
AATTTTCAGAGATAGCCTTATGTACTCCCCTAAACCTTTTTTCCCCAGAAATCTATTTTCAAAAATATACTCTACCA
AGTGAAGAAAGAAACCAAGAAAGACAAAGAATGTTTAGTTGTGATTGGAGATCAGAAGGAGAGTGTTTTAGGCATGA
TTCGCCTTGTTATGCTGCAATAATAAGCAATTCCAAAATATTAGTGGCTAGAAACACAAAGGTTGATTTTGTATCTT
TGCAACATATCCAACACAAGTCAGTCAAGGGTTCTGATCATTGTAGTCACTCAGGAATCTAAGCTGATTCAGTCTCT
AGTATTCTATCATTCTACCGTAACAATAAGAAGCTTTAGCGTTCACCATGTCAGTCAAAAATGCGTGGAAAATTTTA
AACTGACTCTTACATGCCTCTATGTAGGAGTGATATATGTCATTTTCATTTCACTTCATCATCCAAAGGAAGTCACG
TGGTCATGCCTAACTCTGAGGGGACAGGGGATTATAATCACTAATATTTTTAAGAGAGGTAAGAGAATTAGGAAAAT
ATTGGAGCATTAGTATTACCTGCAAAGCCTAGGATCCGGAAAACAGAGAACCCAACGTGGGAGGAAGATGCAAAGTG
GTGCCAGAAAGATGGTGGAGGAGATCCTGTAACAACAGCTGTATAACAGTTGTAGAGAGAAACCTGTACAGATTGGA
GAAGAAAATAGTGCTCCAGGAATTTTGTTACCAAGAAAAAAAAAATCTAATGTATTCGAATGTATTGAGAGGTGGTA
TTCTTATTGTGTAAGAGTATAATATATGACATGTATGACAGACAGAAGTACCATTTAAAATTTTATTAAACATAAGA
ATTAGATCCCTATCTCACACTGTACATAAACATAAAAATGGTAAATTGATGGATTGCCTAAACATGAAAGGCAAAGC
ATTAAAATATATAAAAATATTTTGGGGCATGTAGGGCAAAAGAGGTGTCCCTTAAGCAACAGCACAAAGCAAAAATT
GTAAAGAAAAAGACATATATTTGATCATTTTAATTAAAAACCTGTACAATGCAAAGACTTCATAAACAAAGAAAAGA
CATAGGCTCAGATAGTATATTTGCAATACTAAAACAATAAAGGATTTATGTCATAATTTAAGAAAAATATTTACCTA
TCAATAGAACAAAATAAAAATAACCTAACAGGAAAATATTTTTAAAGTTTAAAAAATAATAGACAGTTCACAGAACA
TCTGTATGTCAAATACACTTATGAATAGATGCTAACTCTCATTTATTATTGGAAAAATTACTTCATATTAGTCGGTT
TTCATGCTGCTGATAAAGACATACATGAGACTGGGTCATTTATACAGGAAAAAGGATTTAATGGACTTACAGTTCCA
CACAGCTGGGGAGGCCTCACAATCTTGGCAGAAGGCAAGGAGGAGCAAGTCACATCTTACATGAATGGCAGAAGGCA
AAGAAAGTCCCCTTTATAAAACCATCAGATCTCATGAGACTAATTTACTATCACGAGAACAGCATGGGAAGGACCTG
CCCCCATGATTCAATTACCTCCCACGAGGTCCTTCCCACAACATGTAGGAATTCAAGATGAGATTTGGGTGGGGACA
CTGCCAAACCATATCGTACTAAGTGAGATAACGTACTCCCATCGGACTGGAACAAGTAAAAAATAAAATAAGATTGT
GAAAGGTCACAAACTCTCATTCTTGTAGAAATTTAAGTTGGTACAACCAATTTAGAGTGTAATTTAGCAATAATCTA
GTTAAACTGAGGATGGCATGTCCTACCAAACAGCATTCCAGTTAATCACATTGAAGATAGCATGATTGTTCTCAGGC
TTCCATATTTTCTGTTGAACGGTAAGTTGTCAGTCTAATTGGCATTCCTTTAAAATAGACTTGTTTCCTCTTGATGA
TTTTCACATTGTAATTTTGTCTGATTTTCAGCACTTCACCGGTCTTTAAATGATTATGTAAGATTTTCATTGCTGAG
GAGGAAATCCTATCACCTCAAATACTATCTTATTAGTTATATTTTCATTTTATTTTACCATGATTTTAAATGAATTC
CTTAAAAAATTAGGTTAGAGAACTAATAAATGATAAAGAGAAAAATTATTCTTCATCCTGATAGCTGTTATGTGAGG
GTGGAATGGTATGGCTGGTTTTTTTCACTGTCATTTTGTAACTTGGAAATTCTTATTTTTTAATACAATGACAATAT
TTTCATAGTTAAAAAACACTAAAACTAAAACAATAGTCTGTTAATGACAAATTTAAGGGCGATTTCATTAAGTTAAA
AATCCATGTTAAGGCATTATAAGATGAATAAATACTCCAAATGAAAACAAAGTGAAGGCCACTTTTATTTACCTTTT
GTACATGATACTTATTTTTCAACAGTTGTCATGTTAGAAAAAGTTGAAAAAATAAAACAAGATACACCCCCTTTTGG
CCTCTAACCTTCCAAAATTGTAATGGGTAGACTTCAAATTAAATATCTAATTCGACTCCTTTCCACTTCTACCCTCA
TAGCATTAGAGACATTTGGAAAAATGAAAAAAATATAGTATTTTATGGCAGGGTACCTCAACCCCAGAGCCACAGAC
CCGTACCAATCCGTTGCCTGTTAGGGACCCGATTCGCAAAGCAGTAGGTGAGCAGCAGCGGGCTAGGGAAGGAAGCT
TCATCTGTATTTACAGCTGCTCCTCATCACTCCATTTCTGCCTGAGCTCCACCTCCTGTCAGATCAGCAGCCACATT
AAATTCCCATAGGAGCACAAACCCTATTGTGAACTACTCATGCAAGGGATCTAGGTTGCATGCTCCTTATGAGAATC
TAATGCTGCATGATCTGTCACTGTCTCCCATCACCCCCCAGATGGGACCATCTAGCTGCAGGAAAGGAAGCTCAAGG
CTCCCACTGAATCTACATTATGGTGAGTTGTATAATTATTTCATTATATATTATAATGTGATAATAATATAAATAAT
GTGTGCAATAACTGTAATGCACTTGAATCATATGGAAACCATCCTCCAACCCGGTCCACGGAAAAATTGTCTTCCAC
AAAATCAGTTCCTGGTGCCAAAAGGGTCAGGGACCACTGTTTTATGGGATAAGTGAAAGCTGGATTTGAATCCAACT
CTACCTTTTACTTCCCATGTGAACATTAAAAGATGACTTAATGTCTCTGAATCTTTTTTTCCTTCTAAGTAATAACT
ACCTTGCAGAGTTATTACTTGAATTGGCCATAAGGTAAAGAGGACACCAAATACAGTAAATAATAACATAATAATAA
AGAAACCTTATGAATTCTATTCAGTGATATCCATATTGAGGTATTTAAGGTGAATGATACTGATGTCAGCAGTTTAC
CTGGATATGCATCAAAAATATGATTGATTGATGGATGGATCGGAGGAAGGAAAAGATATGTGATCAAAACGAGTATA
GTAAAATGTTCATGGTGGAATCTAGATATATAGATATCACTTGTAATTTTTAAAAATGTTATTGCATTTTTGAAATT
TGTTATGTTAAAATATTGGGTGAAAATTCTGATGCCCTGGCCATGTCCCAAATCAGTTGAATCAGATCTCTGGTGTA
TTTTTTTTTTAAGATCCTTGGATGACTCCATTGTACAGCAAGTTTGCAAACCACTATTCTAACTTCTCACCACCCCC
TTCTTTATAGCTTACAAAATTATATTTTTAAATGTCAAATGTCACAATTTCACTTTCCTTCTTCAAGAACTGATTAG
ACTTCTAGCTTCTCCTATGCTCTCATCTTGCATAACGTTCATTTGCACGTGGTTAAAACTGATAGGCAATAAATAAT
AACCCATCTGTCTTTTCCTGACCCAACATAACTAAAACCTTCCTTTCAAGGCGAAAATGAACTATAATCTCACAGAC
AAGAGTAGCTATAGACAACTAATTCTTTCTGGTCTCTCGTGGTCATGGGACTATTCACCACTGCCGTCCCTGCTGTC
TATTTGGTGGACCAGCCAGTTTTACTGTGACCCATACCATGGCTCTTGAGAAGCATGAAGCTGCCGAAATCGCAGTT
GTAGGGTTAATGGAAAACATGGTGAAGTATAGCTTACATATATATACAATTTTGGCACAAAATGAAGCCTAAGAAAT
TGTACCGGGCTGGGTGTGGTGACTCATGCCTGTAATCCCAGCACTTTGAGAGACCAAGGTGGGCGGATCACCTGAGG
TCGGGAGTTCGAGACCAGACTGAGCAACATGGAGAAACCCTCTCTACTTAAAATACAAAATTAGCCAGGCATGGTGG
CGCATGCCTGTAACCCCAGCTACTCAGGAGGCTAAGGCAAGAGAATCTCTTCCACCCTGGAGGCATAGGTTGTGGTG
AGAGAAGATCACACCATTGCACTCCAGCTGGGGCAACAAGAGCGAAGCTCCGTCTCAATAAATAAATAAATAAATAA
ATAAATAAATAAATAAATAAATAAATAAAAGAAAAAGAAATTGTACCAGGAAGTGGATTGGGAATGCTCTATATATT
CTCCCCCTCCAAGAAGTTTTTTTAATTTATTGTATTGTGAAAGATCTGCAGCTATTTATTTTGTGTGGTACTCTTTT
GCAATAAACTACTTCTGCATAAATGAACTCAAAAACTTTGATATTCATGAACCTTCATAATGTTGATCTAGTACATC
TTGCCCAACTAGCCCTTGGGCCATTCCTTCCATGCCACTGGTTGAAAAATATGCTCCCCACCCCAGTAACATCAGCA
TCAGACCTCTGCAACCAGAAAGATTCCATAGGTAATGAAATCCAAATGCTGGCATTTGGAAGTCAAACCAGTGTATT
TTGAAATGGAAAGGCAAGGGGAAGTGCATGGAGCAAGTACAGCATGTGCTACAACTTCAGTTCATACCCTCCTTAAA
ATACTGTGCAACTAAACTGTCAATTGCCCATTGATAGAGATAGTATCTTTTTCAGCTTTACACCTTCCGAATTCAAC
ACACTGCTAGCCTCAGGACTAAGCGCTCAATAAATGTTTATCAAAAGATTGAGTAAATGAATCAGTGGTGAAATAAA
ATGTACAAACACATACGCTTCATACACGTTTAGGAAGCATTTCTAGTGAGCTTTGTGGATATTTTATTTCTTAAAGG
TTCTTTTTCTTTCCATGTTCATGTTATTCTTTATTTTTTAAAATGTTGCTTCTGCTTATTTCTCTTTCTACGGCAGC
TATATTTACTTGCCTGAGAGCACAGCATATACTGATGTTAATCAAACTTTAGATGAAACACCTAAAATGATAGAAAA
GAATCTGCTCTAATTAAATAATAAAGCAACCCCTACAGGTTTAGACATGTGCCTTCCGGTGTGGGAAAGAAAAAATT
TAATGAATGTAGTAGTTTTATGCCAAGAACATTTCCCTCGTGCACCTGTGTTTTAATAAGATAGAATATAAAATAGC
AAAAGGGGCCCGACTTTTGTGATGATATTTACTCATAAGGTAGTAAGTCAGATGTGATGACATTTTCTTTGACTTAG
AGCTGCTATACTTGGGTCAGATTTCAGTTCAGTAAATTTGCAGTGAAGTTGTCTTTCTAACATGGTGTCATCCTGGA
ACTGCCCTGCTCCCACAGTTACATACAGGCTAGGGAGTGGGTAGGAGTGGGGGTGAAATCCTCTTAATGTTTATGGT
GTCAGTAGATTCAAACTAAAATTAGCCTTACAGCCATACTCCTAATAAGGGGCCCCTGGCATATTTAATTGATTTAA
CAAATTTATCAAAAATAGATAAACTGAAATCTGCCTTGAAATTAATTACTGTATCTCTATTTTTATAAGAAAATATT
TTGGACCCGTTCCCTCTGCCTTATGGGTGCAACTCCCGGCAGAATAGGTCATGTCTCCTGAAAATAGTTCTTATTTT
CTTTACTACTTATGACCTCTTATAGCCTAGAAGTTTTTCCTTGCTATTTCTAGTTGAAATGCTAATCTGGCATAATT
TCTAGTTGAAATGCTAATCTGGCATAATTTCAGAACTAATTTTCTGTTAATGCCACTTGGAACATCTAAATTCCTCC
TTTTTCAATAATACTATATTTGTGTGTTGCAAACACACACAAATCATACTATATTTGTGTGTATATATAGTAAATTC
CAACTTATAAAAGTAAAATTGGAATTTTTTTCTTTCTTTGGAAAATTATTTTATAATTGAGAATTATAAATGTCACT
TTTTTAAATGCTGCAACCTTTGAGATTGGTTTCAATAAAGTAAAACTTAGTAAACATTAAGAAAAATGATAGCTTGA
TATGTTCACTAATATGGTAAATGAAAACTTTTATGTGTGTATAGGCTCTTGTTAATTATAACTACCTTCACAGGAAA
AACAGTCTTGTGGAAGGTAATGGTGCCAGTGAGAGAAAATAGAGAAAGTAGAATAGTAGTAAGAGAGAAAATAGAAT
TTCCCGTTTAGAATAAAGCTAAGTAAAAAGTGAATTGTTTCAGTTTTATTTATGTATTTGTAAGTGTGGTCTCATGC
CATATATATTCAAAGATAGACAGAGATCTTAATCTTTAATTTTTTATGGCCAAAGAAAATGAGTCCCATGTAAAAGG
GACATTTTCGTAAGCTGATCTCAAGTGGTGTATTTTGATTTGCCGCAGCAGCCTATGGAAGAAATATCATTTTGGCT
GCCTAAGAAGAAAAATGTCATACCTTTCTATTTTTTATTCATACTCCGCATATTTGGAATTCGTATTCTCACCATAC
TTTTGGGAAGATAACACGAAATTGCCTTACTTGCCTCGAAATCAAAATCTGCCATTCGTTTTAAAATAAAATGGCTT
TTTCATCTCATTTTCCTTTAGCAGAAAAAAGTAAGTTAAATTTTTTTGTAGCTGTGTTAAGTGTACTGTTCCTCCCT
CGTTGAGAACGAAACAAAGACATATGATTCCATTTACTGTAATTGTTTTGGTTTGCCCAATTACAATAGCAGTAAAA
TCATTTACACATAGTAAAATGTTTGAGCCTTAAAGGGCAAGAAGACTAAATTTAGCTGAAGATATACAACTTTCATG
TGCCAATGAGGAAATGTATAAAATCAGTTATACATATTTTCCTCATTCATAGATATTGAAAATATTCATATATGTGT
ACATGTATTTGATAACTTAATATTTTCTAAATAACAGAAATTATATACTGTATATATATTTGGATATTATGATTATT
GTTATTGCCACCATTGAGCACTTACTATGGGCTCAGAAAACTTCACTGTATTATTTTTAATTCTTATAACAACCCTG
TGAAATATATATTATGCCTATTTCACAAATGAGAACTCTGAAATCAGGAAATTAAATCACTTGCTGAAGTTTACAAA
GCACTGTTAGGCAATAAAACAGAATTTCAAACTCACGTTTGCTTGTCTTCAAATTTTGTATGCTGTACAACCTGATT
GTTTTACTACTTGTTCTTAATTTCAAAAAAAATCTTCATACTAAAAGATGATACTTTGGGAGTTCTAAAGAACATGT
TTTTGGCCGGGCGCGGTGGCTCACGCCTGTAATCCCAGCACTTTGGGAGGCCGAGGCAGAAGGATCACGAGGTCAGG
AGATCGAGACCGTCCTGGCCAACAAGGTGAAACCCCGTCTCTACTAAAAAATACAAAAAATTAGCCGGGCGTGGTGG
CGGGCGCCTGTAGTCCCAGCTACTCGGGAAGCTGAGGCGGGAGAAAGGCGTGAACCTGGGAGGCAGAGCTTGTAGTG
AGCCGAGATCGCGCCGCTGCACTCCAGCCTGGTTGACAGAGCGAGACTCCGTCTCAAAAATAACAAAACAAAACAAA
ACAAAACAAAAAAAACCATGTTTCTTTTCAGAAGGTATACTCATTTGAAGTGGATACCAATTATTTGTATTAAAATT
ACTTATGGATAAATTTGAATCTGCAAAAATTAAGTGCAACATTATTTTTTGGCACCTGTTATAGGAGGTAAAAATGG
TGATTCAATATAGTTTCTACCCTTAGGGAGCATTTAGTGTGGTTTAGACGTAATGACACTAATGTAAATTAGAGTAG
AATAAATTCTTTAAAGCAGGACAAAAGGAGTGTTAAAGACTACAAAGTTTCAGTTATGGAAGATAAAATTCTGGAGA
TCTATTATACAGCATAGTGCCTATAGCTAATAATGCTGTTTCGGACACTTAAAATTTCCTAAGAAATCTTATGTTGT
GTTCTTACCACCAGTAACAACAATAATAAAGGGGACAAGAGGAAATTTTGAGTGATGACAGATATGTTTATAGCCTT
CATGGTGGCGATGGTTTTGTGGGTGTATACTTATTCCCAAACTCTTTGATTTTATATATATATATATATATATATAT
ATATATATATATATATATATGATGAAACTCTTTGAGATATATATATATATATGTAAAATATGTACAGCTTTTTGTAT
GTCAATCATACTTCATTAAGGTGGTTACAAAAAAGAATCAAGAAAAATAATCACCCTAATAAAAAGAGTGTTATTAA
AGAGGGGAGGGATTACTTCTAATTGGTAGTATCAGAAAGGTTCTCATGGAGGAAGGTATAGAAAAATTTAGGACAAT
ATAAGCAACGATACTTTAAGAAAAGGAATAATATAAACAAAAATAAACCAAAAGATGAAGAACCACCAAGGATGTTT
CTGAAAAATCAAGAGAAATTCCATTTTGATTTGAGAGTATGAGGTACTTGTCATGATATATAATGAGAAGTGTCTAG
AAATATGTTATTTTGAGCAAATGCTGCTTAGAAAGGAGGTTAGGTTATTTATTAATCCATTCCTTTGAAATATAAAT
TTCATGATCAGAAACACGTGAGTGTAACTATCCAATAACAACATGATTTATTTCCAAGAGGATAGTATATTATAGGA
GAAGTAGGATTTTTTCTCTTTCACAAGCTAATACAGTGTTTACTGATTCAAATGACACTTAAAAAAAACAAGAAACA
AAAAACATACACTATAAGCTATCCTTATTCAGCTATGTTATTGTCTTGCATTCCTGATTAGAAACATACAAGTTAGA
CTCCCAAAGTCTCAGTTTTCTCATTTGCAAAATGAATAAAATGATAGTACCTACCCCAAGGTGCTGTGGAGTATATT
TAATCATGTACTTAATCTGAAGTTTTAATTAGAGTGCATAGCATATATTTGTGGTTGCTTTTGCTCCTATTTCTGGG
TAAAAGTTCCTACACCTATTATATATGTTTTGTGTTTGAATGAAGAGAAAGGCATCATTATTTCAAATATCCCCTCT
AGCCAACAAATGAAAGCTAAGACTTGGAAGCTAGGATGTCTAACACCATATCCTCTTCATGTGGACTATATAGTAGA
GGATATGTAAATTAAGACTCAATTAAATGCTCACATTTTATAAAGGAGACCCTTGAATATATCAAATCCTCAGAGAG
TATGTCAGTTTAAACTTATTAAGAAATTTAAATTATTTAACTTAGCCTGCTGAGTCAGTTTCTGTAGCTGATTTCTC
CAAAAGGTCTTGGTATTAAAAGTAGCATTACAGATCACTAATAGCAAACTATTTCTGCTACTTTGGGTATTTTTACA
GGGTGCTAATTGTGCCAAATCAAGACATTGAAATTGCATAATTTTAGGTTAATATTAGCATAATCCTACATATTTTT
TCAGAAGCAGTGCAGGAATAGTACACCCTGAATATTTTTCCATCATGATGGAATTTTCAAATATCACAACCTTTACA
TTGTCAGTTGTCTGCTGGAACATTATATTGTGTCAAAGAGATTTATAAAAGAGAAGGGCAAAGAAAATTTGCGGAGT
TCTAGACAATAGTAATATCAGGGACACTTTTGCTTTCTTTGTTATTACTAAGGAGTTAAATAATCAGATACCAAGCA
TAGGTAAAGAAATGAATCTCTAACTATTTGAGTACTTATCAATTTATCTCAAGGATATTTACGGTGTTTTCCCCTAA
GCCAAAATAGAGTGCTTAATTAAGGTCCCCTTAGCCCCCTAAAAATCACTCAGGCCAAGTTTCTGATATGTGTGTAT
ACTTATATACATATTAAAGTATATGTATTTTTGTGTGCACTTTCAGATCAAAAAGTCCTCGTTTATCTAAAGCAATT
TCAGCTTATTGATGAGGCTTTTCTGTTACAGCGTCCTCCTCCACAGATCTGCCTGTCTAGAGATTTAAGCCCAATAA
GAGTCCAGCAAAGTTTAAACAACACTTTAGAAATCTAAAGAATTTTAAGATTTATTTTAATAGTCTTAGTGATAACC
TGAATTGTTTGTGAGTTCTTACAAATAATGCAATCAACATTTAAGTAATTTTATTATTTTTTTGTTTGTATAAATTT
AAGGTATACAAGTACATTTTTGTTACATGGATATATTGCAGAGTGGCGAAGTCTGGTTTTTTGTGTACCCATCATGC
AAATGATGTACCTTTTCTCCATTAAGCAATTTCCTATCCTTCGCCCCCATCCCACCCTCTCGCCCTTCTGAGTCTCC
AGTGTCTATTATTCCACACTCTGTGCGCATGTGTACACATTATTTAGCTTCCACTTGTAAGTGAGAACATGCAATAT
TTGACTTTCTGTTTTTGAGTTATTCCACTTAAGATGACCACCAGTTCCATCCATGTTGCTGCAAAAGACATGATTTC
ATTCTTTACTATGGCTTTGTAGTATTTTTCATTGTGTATATGAAATTGTTTATTCCATACGCAATTTGTGTGTGTGT
ACATATATATATATATATATATATATATATATATATATATATATATATATGCTTAGACTTAGAAGCTAGGATAGACA
CACAATGGAATACTACACAATGGAATACATTCATTCACACACATATAAATAAAAGAATATGTGGAGATATATCTCCA
CATATTCTTTATCCAATCATCTGTTTTTAAATAATGCTATTGACTTCTTTAGGGTGAATTTTATCAATATTGTTTTG
GTTTAAAACACTCACCTTAAAAGAGTCACAGTCCCTAAATGTGCATCCTCATATTTAAATTAGGTCTCAGTAAATTT
GTGCAAAGTGTATTCTTTTTAGGATGGTGTTGAACTTGCTAAATTATTTATCTTTAAGAATCATCATTTTGTGTCTT
TTATTAATGAAAACAACAATTATGTGATTGCTGATATATTTGGAAAATGATTTCTGATGTAGATTGATTTTTTTATT
CTAAATTCTGTGTCGGTATTAAAAATTTATAGATTACTAACTGTATTAATATCGATAATACTAAATTTTATTGCTAT
TTATAACTTGGAGTGTACTTTCATCCTCCTGAAAAAGCTGAATGAGGTAGGCAGTATTATTCTGGGTTTATGTGTGA
GATAACTGAGACTCAGAGGTAAAATAGTGTATCCAAGCATTCATGGCTCTTAAATGGAAGATATAAGGGGTTTGTGA
AATTACTCATGGACTTTTTTATTCATTCATTCAGTTATTAAAATGTATTCAACATTTATCATGTACCAGGAACAGCG
CTTAGTACCAGGAATTCAAAGGTGCATAAAACATCTTCCTTATTCTAAGAGGTACATAGTGTACTGGAACAAACAGC
CTTGTAAATACATAATTAGAACATGAAGTAGTATGTTAATAGAGGTTTTCACAAAGCTGTGGAAGCTTGTCTTATGA
AGTAACTAATTCCAAGGGAGAGAAGCCTTATGGAATAGTGACATTTTAGATAGGGTGTCATTCTAAAATACAGCAAA
AGGCCCACAGTAAAAAAGGAATTTTGGTTGTTATGAAAATTTTCAGATTTTCTATGTTTTCAGTACAGTATACATGG
TGGGCTATGTGAATGTTTGTATAGGGACCAAAGTAGGAAGTGAGGTTGTCTGTTAGAGAGCGCTGAGAAACCGAAAA
TAGGGAGAGATGAGTTGGAATATGCTGAGGAAAAGTTATTAGGAGTTTTCAAGAAAGGCCACGACAGTGGGGCTAGA
GAGAAGAGGCTAAATTAAAGAGTCATTTCTGGTTTAGAATTGATAAAATATAGAGACAAGCATGATAAGAAAGAAGT
CGAGAAGTAAACGATGGTCTCAAGATTTCTAGCTTGGAAATCATTGACTAAAATTAAAACTAAGGACTGGATTAGGC
CATTCTTGCATTGCTATAAAGAAATACCTGAGACTGGGTGTTTATAAAGTAAAGAGGTTTAATTGGCTGACGATTCT
GCAGGCTCTACAGGAAGCATAGCAACATCTGTTTCTGGGGAGGCCTCAGGGAGCTTTTACTCATGGTGGAAGGCAGA
GCAGGTGTAGGCATTTCACATGGCGAAAGCAGAGAGAGAGAGTTGGTGGTGGGGGTGGGTGGCTACCTACTTTTAAA
CAACCAGATCTTGGAGAACTCACTCATTTTCATGAGGACAGTACCAAGAGGATGGTATTAAACCGTGAGAAACCACC
CTGATGATCCAGTCACCTCTCACCAGGCCCCACCTCCAACATTGGGGATTACAATTTAATATGAGATTTGGGTGGGG
ACACAGATCCAAATCATATCAAAGACTTGCATGGGAAAATAAGGAATTGTTGACATAACATCTTTGAGGTTCACATC
AAATGTTCTGATGAGGATAGTCCAAGTAGCAGTTGGCTATATACCTCAGATAAGGGCTGAAATTTGGAGCTATGTCA
TAATCAGCCTAGATTAAGAGTCAATAATCTCCTGCCCATGGGCCAATTACACCCACCACTTGTTTTTGTAAAGTAGT
ATTGAATCCCAGCCATATCCATTTGCTTATGCTCCATGTATACCTTTTTTTTGAACTTCAAGGCAGAGTTGAGTAGT
TGTAACAAAAACCATACGGCCCACAAAGCCTGAAATATTTGTTCTCAAGATCTTTATCTATAAAGTTTGCCAATACC
TGCTGTAGATGTTAGTTGAAGCTTTGAAAGCAAATGAGGTTTCATAAGGCAGTGTCCATACAAGACATTTAACAAGT
TTACCTATAAAAACTAGAATTCCTTTGAGGGGAACACATCCTAGTCTCCATTAAGCACAGTAGAAGAGTCCCCTATA
ATGGGAAAGAGGTCACTTTAGGTGTTGATGTTGGTGGTACAGGTCAAAGAAAATTTATCTTTGCTGTTTATTCAGAA
TGCAATAAGTGAAGTTATGAGAAATAAGGGAAAAAATGTGTAGAATTTCAACAGCGAAGAGAGGGGATAAAGGCATG
AGAATGAGTTCCTAAGCTCAAGTATTATAAACACTGTGAGAAACTTAAAATCAAAGTATGACTCCAAACGTATTTGA
AGCCTGAGAACAAGGCTCACAACCTAGGGAGGATTAGGGATCAATAAAATAGAGTGTTACAAAGTATAATGTCAATC
CAGAGTTGTAAAAATATCAGCATTGAATATATTGAAAGCAGTAAAACTGAATGAGGAGACTATCATTTTATATCACT
GTGTTTATTTCTTTGCCTTGTTCTATAAATATTTAAAATTATAAAATTTTTATTAACAGTGAGAGCAGAACTACCAG
AGTGAGCAGATCAAAATTGGGACAGATGCTTTTCACTGCACACACTTTTATTTTTCTGCTGTTCATGCATTATCTTG
TACAGTGCACATGTTTTACCTAAAAAATTAAAATGGAGTCTCCTGCTTAGGAAAAAAGTATATATTCTGTTTCAAAC
TATATACAAAAATAAAATCCCAGGTGACTAAAAACTGACATGAGAAAAAAACAAATTGATAAAGCTTTTACAGTAAA
ATAGAGGAGAATATGTTAATTAATATAGGGTAAGAAAAAATTGCTTACACAAATGATGAAGCACTAATCATGAATAA
AAATAATAAAGTGGACTACCTTGTATATTAATAACATCTATACATCAAAAGACAGCACTGAGAGAGTAAAAATGAAA
CCCACAGAGTAGGATAAATTATTTGGAATACACACATAATGGATGAAATGTGTGTATTCATAATTATAAAGAATTCC
TACAAATCTTTCAGAAAAGAACAGATAATCCAATAGAAAAATGGGAAAAGTTCTTGAAAAGTGAACCATGGCACAAA
AAGGGCTTGTGGCCTGCTGGCAATATTCTGTATCTTGACCTGGATGGCATTTTTAAGGTGATCACTTTATAGTAAAT
AACTAATGTGTTTTATGCATCATAGTAACGTTAAGATTTTTGTCATCTTTACAAAATAAGAAATCCAAACGGCCAAT
AAATATATAAAGAATTTCTAAGTCCCATTAATGGTCCAGGCCATGCAAATTAAAACTAAAATGAAATATCACTGCTT
ACCAACCAGAATCATTGAAATTTATAAGTCTGACAATTCCATGTGGTGGTGAGAATATACAGCAATTAGAAATTTCA
CACAATGTTACTTGGTCTGTGAATTGTAAATAGAAGTGTAAAATTACACTACTGCTTCTTGGAGTGAAATCCATTTG
GCACTATTTAGTAAATTCAAAGATCTGCATAACCTATAGCCCACCAATTTCACTTCTATATATACACTCTACAGAAA
TGCATATGTTCATATTCCAGGAGACATGTTTGGGAATGTCATAGCAGCATAGTAATAGCCCCAAACCAAAACTACTT
CAGTATTTATTAATAGTAAAATTTGCTATAGTTTGAATGTGTCTCTTTCCAAATTCAGGTGTCGATAATGTGCTAGT
ACTAAGAGGTAGGGTGTTTAAGTGGTGATTAGGCCATGAGGGCTCCTTCTTTGTTAATAAAAATAAGACCCTTATAA
ACAAGGCTTCACGCAGCATTCAGTCAGCTTGCTCTCTTGCCCTTCTACCTTCTGCCTTGTGAAGATACAGCAGGAAG
GCCCTCACCAGACACCAAATGCCAGAGCCTTTATCTTGGACTTCCCAGCCTCCAGAACTGTGAGTGAATACATTGGT
ATTATTTGTAAATTACCCAGTCTCAGGCATTTTGTTATAACAGCACAAACAGACTAAGACAATCATACAGTGAGAAA
TTAATCAACAACTAATAAGCAAAGAGGTAGATTAATCTTGAAACTATGATATAGAGTGTTCCATTTGGCTGCTGGAA
GTTTTATTTCTTGGTCTGGGTGATGGTCACCATGGGTTTATATGAATGGTTCCCTATATTATGTTTCACAACAAAAA
GCATTTAAAAAGTAAATATATGTAATGTACTCAGGGATAGGCATGGCCAACCATGGATTCTATGCTGAAATAATGAT
TCAGATTTCATCAGCAGGCTAATGACACTGCCTATTTAAATACTTTAAGTCCTGAAATTAAAGAAGGTAATTTCTCA
AGAAGGAATTTCTAATTTATGGGTGGGTCTATTCCCCACCAGAGAGACACTAGCATGGCTCAGATTCTATGTTGGTC
ATTTTATTTGCATTTAAAGTCTTAAGCCAAATAGAGGTACACTAATAATGACAACAACTACTACTACTCATACTTGT
GGAACACTGCCAGATGCTGTTTTAAGAAATTTGCATTTTCATTTGTAACTGAGCTTACTTGAATCTTCTCTCTTTTT
TTCTTGGTTAATCTAACTACTGGTCTATCAATTTTACTTATCTTTTCAAAGAATCAACATTTTGTTTCATTGATCTT
TTATATTTTTGTTTCAATTTCATTTAGTTCTGCTCTGATCTTTGTTATTTCTTTTCTTCTGGAGCTTTGTGTTGGCT
TTGTTGTTGATTCTCTAGTTCCTTCAGGTGTGATGTTAGGTAGTCAGACTGTGAACTTTCAGGCTCTTTGATGTAGG
CATTTGGTGCTAGAAAATTTCCTCTTAGCCTTGCTTTTGCTGTATCCCAGAGGTTTTGAATAGATTTTGTTGTGAAT
GTGATGAAAACGGAACATTTGTACACTGCTGGTGATTGTAAATTAGTACAACCTACATGGAAAACAGTATGAAGATT
TCTTAAAGAACTAAAAGTAGATCTAACATTTGATCTGGAAATCTCACTACCGATTATGTACCTAGAGGAAGAGAATT
CATTATATCAAAAAGACACTTGCACGCATATGTTTATAGCAGCACAATTCACAGTTGCAAAGATATGGAACCATCCT
AAGTGCCAGCCGACCAATGAGTGGATAAAGAAAATGTGGCATATATTTTCATATACCGTGAAATACTATTCAGCCAC
ATACCATGCAATACTACTCAGCCGTAGAAAATAATGAAATAATGTCTTTTGCAGCAACTTTGATGGAGCTGGATGCC
ATTATTCTAAGTGAAGTAATTCAGGAATGGAAAACCAAATACTGTATGTTCTCACTTATAAGTGGGAGCTACGCTGT
AGGTACACAAAGGCAGACAGAGTGGTAGAATGGACTTTGAAGACTCAGAAGGGGCAGAGTGGGAAGGTAGTGAGGGA
TAAAAAATTACCTTTGGGGTGTAATGTACACTACTTGGGTGACACGTGCACTAAAATATCTGATTTTACTTCTATAC
AATTCATTCATGTAACCAAAAATCACTTGTATTCCAAAGACTATTGAATTTGAATTTTTTAAAAACATTAATAAAAT
AAAAGATGTAAAAAAAGAAATTTATATATACTCATTTATTGAGCTCCCACAATTAACCTTAGGAGGTAAGTACTTCA
TAATTGGTAGTATACTTATCTTTTACTAAATATTTGTATTACTTGGGAAGTTGAGGGTTGGGGAGAAGTAGCAAGGT
ACTATGATTTGGGGCAGATAACTAACTTATTTATTCGCACATACAGTTTGGACCATGAGACACGAGCTCAGGTCCCT
CCTCCTCACCTAATCAAAGATGAAATATGTGGGATGGGATGAAATAATCAGCAGTCCAATGCTGAGTTTCCAGACCG
AAGTATAAAGCAACAATGGATATGTCAGAAGTCTACTAGGGTGTTATTTATTTAAATCTATTTCATGGAATTTACTA
CCACCTTAATGGCCCGAAAGTGTTAAAGTATGCCCCAGAGTACCGAATTACTCCCTAAATGTAATTTATGCTTGAGA
ATAATCTGACTAACTTGATTTAGAACATCAGAAAATAAGTTATGCTGCACATAAATGAAGCAGCAGTGTAATTTTAA
ATACCGGTTGCACGGTGAATGAGAATTTTAATATTTGCAAAATTCTAAAATCACTTGATTTATTATCCTTATGTTTA
TACTGACATTTTTTTGCCCTTTGTTAAGTTCCATCCATATTTCTTCTTACTGCCAAGAAAAAAAACTTTTTTTCCTA
GAAATATTACAGAAGGCAAAAATTATATTTGTTTCCCTGAATGCTATTTTTGATGTCTCTACTTGTTTCTCATTGTT
ACCATTTGCTTCATTCATGGGCAGCCCAATTAATGGAGCGAGACAAATTTAGGGAGCACAGTGACTAATTAGATATT
AAATTGGTAAATCTAACTTTGTAAAACCAGAAAAAATATATATATATTTTTTTCATTTGGAATTTTCCTTGGTGGAA
AAGAGTTTAAAAGTAGTCATGATAAAAAATGTAATTTTACGTAGTAAATTCAAGAATAGATTTAGACTGTGCTATTA
ACAGCACCTATTAAATACTGAAAAGTGTATTTTAAAATTTTATGTGAGGCTTGAAATGGAGTCTAAAGTATTATTAC
TCACATTAAGTGTCATCACATGTAAAGCCCATGATTTTATTCTTTAATATTTTGTTTGAATAGTTACTTATTTCAAC
AGTAATTTCAATAATAAAATTAAATCAACTTTACAGTTTTCAAAGGTTTAGCAGTTGCATGCTGTAATAAATACTTC
ATATTTATATATTTATAAAGTGACAGCATAAGTCATTTTTATTAGGTCCTTGAGGATGCAAAAGTTTGGATTATACG
AGGAGACGAGAGAAAAAGGGAAGAAGGGCATTTCAGAAATATGCTACCGATATGCAAATTCACAAGTCCTAAGACAG
TAGCAGGGGTCGGGCAGAAAGTCCATCCTGCCTCCCTCTTGTGGGCCTGGAACAATGGTGTAAGTGGAAGGCCTGTT
CCCCTTCTCTTCCTACCTCCAGCTCTGTCTTACAGAGCTACGGATACCATGAGCAAGTGTATGAACCCTTACGGTTT
TCTTCTCTTGGGAGAATGTAAAGGAAAGATAACTTGTAGAAACTTGTAGATAACTTGTAAAAAGGAAAAGAATTCAG
GGTGAGAGGGGGATTTGTTGAATTTGATAGAGGATGGCAATTACCAATATGATGAGTGATTGAGAAACAAGTCTGTG
CAACAGGTTTGAAATCGAAAATCTTTGAGGTGTACAGGATCCTGAAATGAAGAATGGGCATTTATAGCAGTATGTCA
GAGAAACAGTCACCTCCTAGTAGCTAAAAGTGTTGGCAAAAGTATAGTTCAAGTGATTGGGTAGGAAAAACAGCAAA
CCAAGAGTGGAGACTGATGGTTGCTACAAAGGTGGAGTGGTAAGTCGTGACCAACTGGTACTTCTCTGTGCTCTGGT
TAGCTGCTGACTGTTTCTCAGACTGTGGTAGCAGGAGGAGGGTTGGAGTTAGCAGTCATTTGCATATGAGACTGCCA
TTTAAAAAAAAATTTTAAATTATTTCATTTTTCTGACTCTCAATATGAAAAGCACATTGTAGACAAATTGAAAAATA
TAGAAAAATTATATAAGAAAATATAGTCTCACCAGTATGGAACAATGCTAACTATGTTGCATAGATTTTTAGATTCT
CATTCAAAAGCAACTCTTTGACTCCAGTGATGCAAATGCATGTAACATATGCAATGTGCAATTCATTTTTAAAGGGA
ATAAACTTACGATATATTCATAGGTCATTTATTGTGTGTTATATACCATTGAAAATATATGAATGCTAAATTATTAG
TAAACATGCAAAAACATTGGCAAGATCATTTTGTTGTGGAAGGATATATTGTATCTGAATAACTCTAGAATACCATA
AATCATCAAAGGCAACATTCTTATTTTTCACTAACTACAGTTAGAGAATACCTCTTCGGCTACCTTCGGTTGCCTTT
TTTATGCTACCAAAATGCTGTCTGTTTTACAAGATTTTAAAGGTTAAGCATATAATTATTCATTAAATACAATGAGT
GCAATGTACATGTAGATACATTATTAAATTTTGGGTAGTTAATAAAAATAAGGGGAAAAAACCTCTAGAACTATCAC
TTTTAATTGTTTAACTGATAAAGTGAAGCTTCATCTTGGAAAAATAATTTCACAAGAGAGCATGTGCACTGGTAGAA
AAGTGCCATTGAAACAAGAGATATTTGGGTTAGAAGCCTCTCTCTACTATTTAATACCATTTTCACCTTTTGGCAAA
TTACTTGGCCTCTGTTTTCTCCAATGGAAAATGGGAATAATAATTGTTATGCTGCAGGGTTATTGTAGGTGTCAATG
AAATGATGTGTCTGGCACTATAAAAGCACAGAGCCCGGTGCCTGGCTATTAGTAACTGTTTAATAAATGTTAATTCC
TTTCTCTGCCCAGGACATCAGTAGGCAGATGTAGCAATTTAAAACTTCTAGTGTTACTTTAAATTCCTGAATGAAGG
TAGAGGACTGAAAAGATATCATGGTATTCAAAAGTATGATCCATTGCTTCTTAAGAATAGAGTTCAGAAAAGCTTGA
CAGATTCCTGTACTCTGAGGCAGCACCATAGCCGGTAATCTGTAGGATGGCTATTGGTTTTGTGCTCACAAATGCTT
GCTTGGGCAGGCCCCAGGAAATCTGGTAGACTGTAAGCCCAGTAAGATTTCAAATCTTACTTTACGGCAGTGTTTTT
CACCTTGACTGTACATTGAAATCACCTGGATGCTTTGAAAAATAACAGCGTCAGTGTCCAACCTCCAGAAATACTGA
TTAAGTTGGTCTGGAATGGAGCCCCAGGATCACTGTTTGGTTATTGTTGTTGCTGTGTTTTAAATGCCCCAGTTGAT
TCTTATGTGCAACTGTCTTAGGTAAACATACAGCCCTGGTTCATATTATTTCTGCCTCAGTCTCTTTTATGACTGGA
AGGTGACCAAATGCTTGTTTCCTAATATTCTTTCCATGTGTAGTATTAACACATTTGACTTGTACTAAGTTCCTGCA
GTATTCCAATCTAAAATTTTAGTGACTACAATAAAATAAGAAGGATTAAAGAAGGCATCGCATAGTTTAGTATATCG
GTTATTTAATGCTTACATGTGAGCCTACAATATGAATTATATCTGTCATCTTATTTTAAATATTGACAGAATCTTTA
ATGATAGTGACGAATTATTGATTTATTGGTGTGATAATGGTATTTTAGTTATATTTTTAAAGTTTTATTTGTAATAA
CTATATGTATTTATGGGGTACAGTGTGACGTTTCAGTGTAATGTTTCATTGTGTAATGATCAAATCAGGTTTCTTGG
CAGATCCATAGCCTCAAACATTTATAATTTCTCTGTGGTGAGAAAATTTAAAATTCTCTTTCACTATTTTGAAATAT
ACAGCACAATATTGGTAACTTTGTTCATATTACTATGCAATAGAACACTAGAACTTATTACTCCTTTCAGTTGATGA
ACAGGCAGTTTTGGATCAAGAATAATATTGAAAGTGATAGAATTTATGAAGTAATTTTTATCCAAAAATATTTTGAA
AGGGAATATATTGCTTCCAAATAATTTATTACAATGTTAAGATATTTGTAAATTTCTAGAATTAAAAAAATATATTT
TTAGGAAAGAAAATGCCAATAGTCCAAAATAGTTGCTTTATCTTTCTTTTAATCAATAAATATATTCATTTTAAAGG
GAAAAATTGCAACCTTCCATTTAAAATCAGCTTTTATATTGAGTATTTTTTTAAAATGTTGTGTGTACATGCTAGGT
GTGTATATTAATTTTTATTTGTTACTTGAAACTAAACTCTGCAAATGCAGGAAACTATCAGAGTGATATCTTTGTCA
GTATAACCAAAAAATATACGCTATATCTCTATAATCTGTTTTACATAATCCATCTATTTTTCTTGATCCATATGCTT
TTACCTGCAG

Homo sapiens dystrophin (DMD), intron 43 target sequence 1 (nucleotide positions 1057082-1057131 of NCBI Reference Sequence: NG_012232.1)

(SEQ ID NO: 270)
GTAGGTAACACATATATTTTTCTTGATACTTGCAGAAATGATTTGTTTT
C

Homo sapiens dystrophin (DMD), intron 43 target sequence 2 (nucleotide positions 1127297-1127546 of NCBI Reference Sequence: NG_012232.1)

(SEQ ID NO: 271)
TTTTAAAGGGAAAAATTGCAACCTTCCATTTAAAATCAGCTTTTATATT
GAGTATTTTTTTAAAATGTTGTGTGTACATGCTAGGTGTGTATATTAAT
TTTTATTTGTTACTTGAAACTAAACTCTGCAAATGCAGGAAACTATCAG
AGTGATATCTTTGTCAGTATAACCAAAAAATATACGCTATATCTCTATA
ATCTGTTTTACATAATCCATCTATTTTTCTTGATCCATATGCTTTTACC
TGCAG

Homo sapiens dystrophin (DMD) intron 43/exon 44 junction (nucleotide positions 1127517-1127576 of NCBI Reference Sequence: NG_012232.1)

(SEQ ID NO: 272)
TTTTCTTGATCCATATGCTTTTACCTGCAGGCGATTTGACAGATCTGTT
GAGAAATGGCG

Homo sapiens dystrophin (DMD), transcript variant Dp427m, exon 44 (nucleotide positions 6535-6682 of NCBI Reference Sequence: NM_004006.2; nucleotide positions 1127547-1127694 of NCBI Reference Sequence: NG_012232.1)

(SEQ ID NO: 273)
GCGATTTGACAGATCTGTTGAGAAATGGCGGCGTTTTCATTATGATATA
AAGATATTTAATCAGTGGCTAACAGAAGCTGAACAGTTTCTCAGAAAGA
CACAAATTCCTGAGAATTGGGAACATGCTAAATACAAATGGTATCTTAA
G

Homo sapiens dystrophin (DMD), exon 44 target sequence 1 (nucleotide positions 1127547-1127601 of NCBI Reference Sequence: NG_012232.1)

(SEQ ID NO: 274)
GCGATTTGACAGATCTGTTGAGAAATGGCGGCGTTTTCATTATGATATA
AAGATA

Homo sapiens dystrophin (DMD), exon 44 target sequence 2 (nucleotide positions 1127595-1127643 of NCBI Reference Sequence: NG_012232.1)

(SEQ ID NO: 275)
AAAGATATTTAATCAGTGGCTAACAGAAGCTGAACAGTTTCTCAGAAAG

Homo sapiens dystrophin (DMD) exon 44/intron 44 junction (nucleotide positions 1127665-1127724 of NCBI Reference Sequence: NG_012232.1)

(SEQ ID NO: 276)
GAACATGCTAAATACAAATGGTATCTTAAGGTAAGTCTTTGATTTGTTT
TTTCGAAATTG

Homo sapiens dystrophin (DMD), intron 44 (nucleotide positions 1127695-1376095 of NCBI Reference Sequence: NG_012232.1)

(SEQ ID NO: 277)
GTAAGTCTTTGATTTGTTTTTTCGAAATTGTATTTATCTTCAGCACATCTGGACTCTTTAACTTCTTAAAGATCAGG
TTCTGAAGGGTGATGGAAATTACTTTTGACTGTTGTTGTCATCATTATATTACTAGAAAGAAAATTATCATAATGAT
AATATTAGAGCACGGTGCTATGGACTTTTTGTGTCAGGATGAGAGAGTTTGCCTGGACGGAGCTGGTTTATCTGATA
AACTGCAAAATATAATTGAATCTGTGACAGAGGGAAGCATCGTAACAGCAAGGTGTTTTGTGGCTTTGGGGCAGTGT
GTATTTCGGCTTTATGTTGGAACCTTTCCAGAAGGAGAACTTGTGGCATACTTAGCTAAAATGAAGTTGCTAGAAAT
ATCCATCATGATAAAATTACAGTTCTGTTTTCCTAAAGACAATTTTGTAGTGCTGTAGCAATATTTCTATATATTCT
ATTGACAAAATGCCTTCTGAAATAGTCCAGAGGCCAAAACAATGCAGAGTTAATTGTTGGTACTTATTGACATTTTA
TGGTTTATGTTAATAGGGAAACAGCATATGGATGATAACCAGTGTGTAGTTTAATTTCAACTTGTGGTGTCCTTTGA
ATATGCAGGTAAAGATAGATTAGATTGTCCAGGATATAATTTGGTTGCTAAATTACATAGTTTAGGCATAAGAAACA
CTGTGTTTATTACACGAAGACTTAATTATTTTTGCATCTTTTTTAGCTCAAATTGTTCATGTTGCAATAGTCAATCA
AGTGGATTTGAATTGTAGCCAATTTTTAATGCCAGAAAATACTGATTAAGACAGATGAGGGCAAAAAACACCCAGTA
GTTTATTAAATACTTTAGATATTTCAAAATGCTGGATTCACAAAAGCAGTATCACATTTGACTTTACAAGTCTTCAT
TCTCAAATATGTTTCCATAGTAAATATGCCCTTTAATATTAAGGAGTTAAGCATTTAAACACCTATTTATATGATAA
GCTATTTAAACACAGAAAATATTTTTAAAACCTTGTGTAATTATATGTGTATCAATCAAACTTGCATGCACACCAGC
GTTGGCATTTGTATAGAGAGGAAATGTATGGATTCCCAATCTGCTTTAATATAGAAGATACATTTTAAAAATAGCAC
TGAAGTGAATTTTGGGCTAATGTAGCATAATGGGGTTTCTGCCTGAGAGGCAGAAACATATTAGAGTTATATAAAAT
GTTTTGGGGTAGATATAGAAACCACTTGCCATTTTCAATGATATCCAACCCAAGGTAGTTATATATTTCAATTTATA
TTTTATTATCAAATTAGTACTTATTGTGAAAAAAATCAAGTAACATAGAAATTTGTAAAAGTACCTCCATTCTACTC
TTTGGAGGATAGTTGTTCAGTATGAATTTTGCTACATATTTCAGGCTGGGTTTCTTGGAAAGCCATTGTAAAATGGA
GATTTGTATGTAGAAGGTTAACTAGGGAGTACTTTTACGATGAAGCAATTTGTTTTGATGTAACTTGGTGTAGTTTT
CTTCATGTTTCTTGTTCTTGAAGTCAGTTAAGCTCTTGAATCTGTGCATTTAACATTTCATCAAATTTAGAAACCTT
TCAACCATTTTTTTAAAAAAAATGGAACTCCAATTGTACATTTATTAGGCTCCTTAAAGTGCCCCACTACTCACTGA
TGTTATGTTCATTGTCTGTTTGGTCTCTCTTTTCTCTGTAATTTGTTTTATATAATCTCTATTGTCAAATTGACTAA
TCTTTTTCAAAGTCTAATCTATGGCTAATCCCATGTAGTATATATTTTTAACATCAGACATTTTCATCTCTTAGAAG
TAAAAGTTGGGTCTTTTTATTTCTTCCATGTGTCTACTCAACATGTTCAGTCTTTACTTTCTTGACTATATGGAATA
CAGATATAATAACTGTTAGAATATTCTTCTCTACTAATTTTATCATCTGTGTCTATTCTGGGTTAATTTAAATTGAT
TTATTTTTCTCCTCATTAAGTGTGTTGTTTAACTGCTTCTTTGGATGACTGGTAATTTTTGACTATATGCCAGACAT
TGTGAATTTTAACTTAGCGCGTGCTTGATACTTCAAATAAATTCAAATATATTGAAATAAATATTCTCAAACCTCGT
TCTGGAACACAGTTAATTCACTTGGAAACAATTTGATCTTTTGAGAATCTTCCTTTTATGCTTTGTTATGACCAGAA
CAGTGTAAGTTTAGGGCTACTTTTTCCCCACTACTGAGGCAAAACCCTTCTGAGTACTCTCTCTGATGTCCTGTGAA
TGATAAAATTTTTCACTGGGGCTCGTGGGAACAGGTGGTATTACTAGCCACGTGTGAGCTCTGGTGATTGTTTCCTT
TAATTCTTTTGTGAAGTTCTTTCCTTAGCTTTGAGTGGTTTTCTTGCATACATGAACTGATCAAGACTCAGATGAAG
AATAAAATAAAGCTTTCTACAAATCTCCAAAATTTCCTCTGTGTATATATCACCTCTCTGGTATTTTGCCCTGTGAT
CACTAGTCAGCCTTGGGCTGCTGAAACTCTCAGCTTCATCTTTTAACAAAAGCCTCCTGGCAAGGATCACTGTCCTT
CAATGTCTGATGTTCAATGTGTTGAAAACCGTTGTAGCATATATTTTGTCTTTTTTTTTTTTTTTTTTTTTTTAAGT
GTTTCAGGTGTTTCAGGCAGGAGATTAAGTTCAGCCTCCTTTACTCCAACTTGAAAACAAGTCCAAAACAAACTATT
TTGATGTAATTTGATCTTTTAATACATTAACATTACACAATTTTGTGAATATATCATAATTTAAAATTTTCAGAGAA
TGTCTAATGGTCCTCATTTCTTGACAGTGTGGTTTAGTTGAAACTGATGAACATTTTATCAAAACTTTTCCCCTCAA
TTGGATACTTTTTTTTTTTTGAGATGGAATTTTGCTTTTGTCACCCAGGCTGGAGTGGCATGATCTCAGCTCACTGC
AACCTCTGCCTCCAGGCTTCAAGCAATTCTCCTGCCTTAGCCTCCCGAGTAGCTGGGATTACAGGTGCCCACCCCCA
CACCTGGCTAATTTTTGTATTTTTAGTAGAGACGAGATTTCACCATGTTGGTCAGGCTGGTCTAGATCTCCGACCTC
AGGTGGTCTGCCTGTCTCAGCCTCCCAAAGTGCTGGGATTGCAGACGTGAGCCACCATGCCTGGCCAACTGGATAAT
TTTAAAAAGACCATTTTATTTAGTCTATTTTTTCTCAATCTATAGATGAGATAAGAAAAATCATTCTAGATGTCCAA
GGAAAAATTCTTTCAGAAAAGAGCTGTGAATGATATCACAAACCCCCCAAACAGTTAAGGTATTTCTTTCCTGGTTA
TTTTATGTCCAAAATCATGCATATGAACATGTGCACACACATGAGCGTGCACACACACATGAATACATATACACGCA
CATAATGTACCTTAGGTTATCTTTCCATTCTGAGTAATTATCGTAAAATGGGTAAAATCAACCCCGTAAGATACCTT
CATCGATAAGGCAAATCAAAGCTTTGGTAATTTCTGCTATCTTGGCCTTTGTTGATTGACTAATAATGAATAAGAGA
ATGAGTTTCAATATTTACTATGAAATTATTTTAGAAGACAGGATGTAGACAGTGGCTGTTAGCAGGCAATTGTTTGG
CATGAGCCAGTAATGGTTACTGTGAAAAAAATCAACCAAGCAGCCCATATATTAAACAAACACACGCAGAAGCACGT
TGGAGTCTGAAGCCTCATATGTACAATTTTCAGTAAAGAAATAACTTTTAGATATGAAATAAACAAATAGATATATG
TTGTAAACTTGTCCCTATGTATTTTGATCAAATTGCATCATATTTTTTTCACTTTAAAGAAGAGAATTTAGTGCTTT
AACTGAGACTTAGTGTTATCATTCAAAATATACTGACTGCCAATAGCAGTAGAAAGATAATCTGGTTCCATGCAACT
CTATTTTTTTTCCTCTGTCGCAAGTAAAAGACAAAATTAAGTACATGAATTAGTGCTTTTTGAAGATATTCCAGAGC
AATATACCATGCCACTATGGAGAACCTCTCTAAAAATATCCCATTTTTTTACCTGAGAAAAATATTGATCATGTTAT
ATGCCACTCAAATTGGTTTATTAAATTCGTTGAATGATATCAGCATCTCTTAATGCATTCACTAAACAAGCAGTAAT
TGAGTGCATATACAAAGTTTTATCATCCACCAAAACAGTGACAATCCACATGAGGCTCTAATAGAAGTTTAGAAAGG
GGGTTAAGTGGTTAAATGCTGGACTCAGAAAGATTGGATTCAAATCCCAGGTCCTTTAGCTTAATAGTTGTAGAATC
TTGTGAAAATATCTTAATTCTTTTCATGTCTCTGATTTCTCTTCTCTAAAATGGAAATATAAATGAGATGTGTATAA
AGCCACTTGGAATAGCATTTTGCACAAAATAATTACTCATTAAATGTAAGCCCCTATTATAACTAATCACTCTTTAT
AAGTGATTAGTTCATATCAATACAAACTAAGACTTATTTACTGAATTATCGTCTCTAAACATCCACACTGCAGAAAA
ACCAACCTGGAAATTTCATAAAACCTTATTTTTATGTAGTATAATTTCTTCTCAAAGCATAAGGGCTCTTGGATTAG
GAATTGAGGAAAATTCCAATTCAGCCAAACGCATCTGTTTCAGATAGCTGACACTTCTGCCTACTCATTTCCTAGCT
AACAAGAAGAAATGTTAATGGGAGTTTTCAAAGGAAAAGCTGAACACCATGAAGGAAAGTGACACAAATAATGTTAG
CTCATATATTGACAGGGTGAATTTGTGTGCTTTCAAGTCCCTTCAGTGAAAATAGGAAAGTAGAAATTATAAAATGC
CCTAACATTTAAAGCTAGCATGTTCTTGGAGACTAGGAAAAAATAAGTTTTAAAACATGGGCTATGATAGAATGAGA
TGGAAAATGTTTGTAGTTGCCAGTAGAAACAATAACAATTACCATTAGATTAAGTATTTAAACCAGCTGAATATTTT
TATTAATGGAAATGGCATCTGTTTTATGAAATAATGCTGCTGAATGAACCATATTAAAAATGACCAGTATTTCCTGC
AGAACGTTGTCGCAGACATACAAGCCTGAGACCCTAAAATCTTAAGGTATTCCATTTGAAATCGACCTTAAGACATT
AACAGTAGTGGTATTGTTTAGATGAAATTTTTTAGGCTTTAAATCAACAAATGTTAAGCAGACATGGGGAGCGAAAC
ACCAGTGTGTTATTCTGACATGAATAAACTGCTGTTTTTAGGGAAAAAATATAGTCTTGTTAAGGTTAAGCTAATTG
GTTTTCTGGTATCTTTTGCAATGTTAGTGTGTTTTACTGCTCCATAACCTATGTTATATGGTAAATGTGCAATATAT
TTATATATGTTGCTGTAAAGAAATGTAATAAAAAACTGTTTACTTTGTGATATGAAAGTAAAAATTTATTCATTGTC
ATTGAGCATACAGAAGTAAATATGGATTACATATGTCATATTTTAATGTTCACATGGTCCCACCATCAAATGTTGAA
AAACTTATAGTTTAACGTCATATTCTATTGAAGAAAAATACACTCCCTTTTCTCAAATGTGAAATGTCCAGAGAGAA
TGGAAAATTACATATAAAGCATGTAGTTATAGCATGGTGACCCTGCTGTGATCTCTCAGATGAGGAACAAAAGGGAG
AAAGAAAGAGCACACTGGTGCTTTGGAGTTGAGAGAAGGCAAAAAAAGAGTACAAAAATGTCAAAGCCAAGTTTAGC
TGCTCTTCAGCTCTCCCTTTAGCTGCTCTTCAGCTTTACCTTACCATGGTTATTAGTGATTGAAGAAAATTCTAAAG
CACTTTTTAAAGGACCCAATTCTGAAGAGTTTAGATTCAGAGAGCACAATGGAGTTGGAGTGACTCCTGCTCAAAAG
TTTGAGACAAGCGAGTCCATGAAAAGACCGTCCTCCTCTTAATGGAAATACCCAGGTTTTCTCATTCTTCTCGCCTT
GCTTTCAGCACTCGCAGCCCAGAAAGCCCTTATCTAACAGGTACTGCCGTTGAAAGGTCATTGACTTGTACAAAAAT
GATGAGTGCTGAATAGATGTGCATAGGTCACTGACAGTATCTGCTACAGAGAATGAGTTTTCGTATTTTTATTAGGA
TACACCTAACATGGCAATCTACTGCCTCAAAGAACTCTATAGGAGGTAAGTGAATTTATATTAATACAGATTGAATT
AAAGGATAATCTAGAAAAAGGCATATGATGTAAAAAAATCAGACACAAGTATATTTTCTGTATAGTCAGTTTTTACA
TTGTGATTTCACCAGCTGGCTGCTGAGTTTGACGGCTTCTTAACAGCCACACTGCTGAGATTCAAATGCTGATAGAA
ACTTTGATGGAAAAATCACTGGAGTAAATATTTCTACCATCTGTTGCCCTTCACTGGGACCCTAACGTTAAGAATAA
TTCATACCATTGCTTGTCCTTTATATTTCCCCAGCAGTAATAAAATTTCATAAGATTTTGTTTTGTGGTCACAAAGC
TATCCTGGTTTCTGTAACTAGAAGACATACACTAGCATAAGGGAATCAGCCGGAAAATTTACTGCTAAGAGAATTTG
TCTCTAGTCACTTACTTTAAGGTTACAGCAATGTGTAAGTGTGGGAATACATTTTAAAATGAGCTTTTCAAAGTTAT
TAGCTGGTAGTGGCATGAGAGTTAAGTCTCTTAATACAGTTAAACAGTTGGGCACTTCATCCTTGCGTAAATATTGT
TACCCTTTTATTGCTGCTTGGAAACTCCTCTGCAACTTTTTGGCCCCTATCCATCTTTTCAGAAGTAGTAAATAACC
AATTTACTGGGAGTGTGGTACCAGGCAGAAATTCCGAGAGGGGCTTTCAATCCTTGCCCATCAAGTGTATCTTTCAG
AAATAAGTATATTAAAATAATTGGATAATTTCAGTGGCTTGTTATTAGACTTCCGTTGTCCAGCATGGCATGTTTAA
GAAGATGACAGATTTTCATACATTATTGGAAAGAAGCAAGAACAAAAAAACATAACTTACTGTAGTAACCACGGTAA
AGAACTGCTTAAAATGCAGGATAAACATGTCATCCCTAAGGGATTCCCATTCTTAGAGCATGAAATTATCAAGAGAG
TAAGAGACTACAAAAAATGAGAAGAATGCTGATTGCAAATTCCAAATAGAAAAAATCAAAACAAAACTGCGCACCAT
CATTCTGGAAGCAATGAGAAGCAGAAATTGTCATTTAATGAAATGTAAGATTAAAGTTAATAGAAGTAATTTTCATG
AAATAATATTTTGCAAGGACGATGTTCCAGCCATATTGATCTTCGTGTTTTCTTTTCACATCCCTTCTTACTGTTCC
CTAGAATGCTTGTTTCTACCTTTAAATTTGCTTTTCTCTCTACCAGAGGGCTCTACCCTATCTCCAGTTTCTCACCA
TGTCCCAATCTACTCCCTCTCAGAATTTTTGTACACTTCCCTTTATATATATTTGTGCTCTAATTTTATATTCACAG
ATATGCCTTTTGTAACTCCCCCATCTTAAAGAAAGCACACACGTACGCACACATGCACACACACAAAATTGAACTCT
TTCTGGGAGATCTGCTTAACTTTCTTCATAACTCTGTCACTTGCTGAAACTGTAGTATGTGTTTTCATGTTTATTAT
CTTTTCCATTAGAATGAACATATTTTGGGTACTTGGTCTTTCTCGATCACCAATATACCTCGGTACGTAGAAAAATT
GATTCATATATTGAAAATGTAATATTCAGTAGAACGAATAAATACATAAATAAATTTAAAAATGATACTTTTATTGT
ATTACCTGAGACAAATGATCCCCAAGTTTGTCCTTGCTTTTCATAGCCAAAACATTCTCTCTTACATTGAGCTTCCT
TCACCTCTTCTGTGTACAGAGCACTTAAAATTTTCACATTGCCTGATACTTTAACAATATGATGGCCCTGTTCTCTT
ACCCATTGGAGCATATGTTAAATACCAGAACCCATGTAACAAACATATATTGTGATCCTACTGTGTGCAAAGCAGAT
ACTGCTTGCTGCTAGGAATACAGAGCTGACTAAGAGCTCCTTTTCTCTTTATGAGCTCACAGTCTCATGAGTTCAAC
GTCTTAAGGCACAACGTCTAAAGCAAAGGGCAGTAAGTAAACACTCCAGAAAGTACTGGATCTGGCCTAGGACAAAT
GGTGGGTTGTTTTTCCAGCTGTTATTTTTCCTGCCCCCTAATTGACAGTCCTCCATTACACCTCTGGGATACCTAGT
CTGACTTGGGAAAACCTGACTTTGGGAATCAGAGGCAGTCTCTCTTGCTTATATATGAGGAACTCTAATGGATACTT
ACTGTCATTAGAGAAACTCTGCTTCTAGCCTGGCTCCTTTTGTAAAGAAGGTTGAGTCCCCTTGGAGAGCCTGCAGA
ACATAACCATTTGCATGTAATGAACAGTTTGTAATACTTTGAGATTGATGTGCAATTTCTATTTGACAAGGGAAAAA
CAATTAGGATTAACCGTGGTCGTATATCCCAGAATACCAACGTTGTTTCCACACTCTAAGTGTTGTTGGGTCATTAT
ATGAGATTCATAATTTTGTCCTGTTGTACCCACGTTTGCATTACCATTCAGTCTTAATTTATTATACCCTATTAAAA
GTTTTTTTGGTAATTTGTTCTTATTGCTACTCAGGCATTAAAATGTCTGCAGGCTGTGAAAATGAATAAATTTAATG
TGGCAGCATAGTTCTCAAAATCCTGGCTTTACAACTCATAGTACAGGCTTGTATTGTAAATCCTAGTTAACATGGAT
TTATTTGAAAATCCAATTTTACTGCTAATCTTAAATAACACATTTTTCAAACATTTTATCCTTGAATTTCTATTTTT
TTATAATTTATGGCTGTTGTATGTATTTACAAAAGGACAATGTGTGTACTTTTAAATACTAGTAATGGATTGCTGAA
ACAACTGTAACTTTAAAACAATGCAATTGTTAAAAAAATAAACTGTGCAGCCTGGCTTAATGGAGGCTTATGAACAT
ATGATTAAGATATATGCTATAATAAGCAAATTCACTCAACTGATAGTTCATAGGAACTTTCAAATTTAATCTCATAA
CCAGTGCTATCCTTCAAAGAATGGTCAGGGCAATTTAACGAGTACATGACCACGCAAGATAATTTCATTGAAGAGTG
GCTGAACTGTTGAAATATTTTCTAGTCTCCTTGGGATATCATTAAGAGCAGAAATTTTGAAATGGAATTGTAATGAT
GTTCAGAAAAGATAAGTAGGTAACTCTCTTAATACGTTTTGTGCTGCTGTAACAAAGTACCTAAGACTAGGTAATAA
TTTGTAATGAACAAAAATGTATTGGCTCACAGTTCTGGAGACTAGGAAGTCTAACATTAAGGTGTCAGCCTCTGGCG
AGGGCCTACTTGATATGTCATCACATGATGGACGATTAGAGGGCAAGAAAGATCAAAAGGGGGCTGAACTCCCACTT
TTATAAGGGAACCAAACCCACTCGTGAGGGTGGAGCCCTCAATCCTTAATCACCTCCTAAAGCTCCCACCCCTTAAT
ACTGTCACAATGGCAATTAAATTTCAACATCAGTTTTGGAGGGAAAAACATTGAAACCATAGTAGTGATACTGACTA
CTACCACACAGGGCTTGGGAGGCTACCCTAGCTGTTGCACCCAAGAGATGAATCTTCTAATGTGATTACCTTTATCA
TTTTTTTTACTTTATTAAAATACTTTTATTTTACATGTATACTTTTGTCTACCCACCATTTCCATGTCTGACCACTG
CTACTACTATGTCCTAGCATAACATTCCATACATCCTTAAAACCAAGCAAAGGGTGGAGTTCCATCTTTAAAAACTA
AACAGGCATTTTGGACAACACATTCTTGGCAATGGAATCTGGACAACATTTATCAAACATGGTAGGGAAGGTTCTCA
CTCTGCATTATCAAAACGACAGCCAGATATCAACTGTTACAGAAACGAAATCAGATGGAAAATTTTTAACAAATTGT
TTAAACTATTTTCTTAGAGAGACTTCCTCCACTGCCAGAGATCTTGAATAGCCTCTGGTCAGTCATCTGGAAGCAAT
TCTTCACATAATTCATGAACTTGGCTTCCACTTTAGGAAGAGAACCACCTTTTTCTATACTTGCTTGCATTTTTGCT
TTAATGTCTTCTACAGAACTAGGTCCTTTGGGTGTTTTAGGAGTTTTTCCTTGTTTTGAAGGATTCTTGTCCTTTTG
ATCTTGGTGTTGACGGTTTTGAGTCTTTTCCATTCCGATTTGACTTTTGTGCATTTTTGGCTGGAGTATCTCATATA
GATTTCTTCACTGGCGCTTTTTCTTCAGTTTCCTCATCATCAAAATCATCATCATCATCAAAATCATCATCTTCATC
AGCAGCAAGTTTTACTTTTTTCTGTGGAACCTTGCTACCACCTCCAGGAGCAGATCGCTTTCCAGATATACTTATGA
GTTTCACATCCTCCTCCTGTTCGTCTTCTGACTCTGTATCTTCCTCCCCAGCTACTAAATGCTGTCCACTCACATGC
ACTGGCCCTGAACCACACTTCAACCGTAAGACCACTGATGGTGTTATTTCAAAGCCCTCAAGGGAAACCATGGGCTG
TACAGACATTTTCAAAGCTGCCAGTGTTACTTTAATTGGACTGCCTTTGTAACTCATTGCCTCTGCTTCAACAATGT
GCAATTTATCCTTTGCCCCAGCCCCTAAACTGACCGTTCTTAAAGATAACTGTTGCTCAATTTCATTATTATCCACC
TTAAAGTGATCATCTTTGTCGGCCTTTAGTTCACAACCAAAAAGATAGTTTTGGGGCCTCAGAGGACTCATGTCCAT
CATCGTCCATCAGGTGGCAGGACGCACTTAGGTGGGAGAGAAGGCAGATGATGATAAAGGACCACTGCTCAAGAGAA
CAGCTGTGCAGGACAGAATCACACCAGGGAGATTACCTTTATCTTAGAAAACCTGAACATCTTGTGTACTTTGACAC
TTCTCTACATTTCACCTAACCTTTAACATCAACACATTTATTCAGAAAACTTTTACTTTTGGAGCTGCTCTGTGTCA
GGCTCTATGCTAGGTGCTCAGGATATTGAAATTGATACAATCCTAACCTATTCACATATAATCCAAGGTTTGCTGAA
ATTGATGGACATTTAAACAATTGAAACATTTAAGTGGTATAATTAGCAAATGGACATTTAAGCCATAAAAATAGCAT
CTAATAGATATAATAGAGGTCGGTACACCATTGATGAGTCAGAGCAGAGGCAACCCAAAGAGTAACTAGCCAGAAGA
ATTGGGAAAGCTTCATAGAGAGAGCGATATGAAAATAAGGGAGAGAATTGTAAATCCATGAAAATGAGAAAAAGTTG
AAAAGTGATGGTGTCAGAAAAACTTGTGGTATGATAATGACAAGATGAGAGGAACTCTTGGTAAGCGTGTTGGATGC
ATGGAAAGAAATGGCACAAAATAATGCTGAGGACATTTTTTATTTTATTGTTGGTTTTGTTTTGGTTAATTTCATTT
TTTAAATCTAGTATGCTAGTGTTCATTGTCCAAACTGTGAATCATAAACTCAGTTTGTGGATCAACACCGGCCTTTG
ATTTTTAGTGAAACAAAATAGAAAATATCAGCATTCATCACAAATAGATGTTTCACAGATTTTTTGTTTTAATTGCG
ACTGTGTGTGTGTGGGTGTGTGTGTGTGTGTGTGTGTGTGTGTGTGTATGTGAGAGAGAGAGAGAGAGAGAGAGAGA
GAGATGGCTTGGATGTTTATCACCTCCGAATCTTATATTGAAATGTGATTTCCAATGTTGGAGGCAGGGCCTGGTAG
GTGTGATTGGATCATGTGGGTGGATCCTTCATGAATGATCCCTTTGGTGACAAGTTAGTTCATGCTATATGTGGTTG
TTTAAAAGAGTATGAGACCTCAACCCCCACCTGTTTCCTGCTCTCCCCTTTGCCTTCCACCATGGTTGGTTGTAAAC
TTCCTGAGGCTCTCACCAGAAGTAGATGCCAGTGACATGCTTCCTGTACAGCCTGCAGAACCGTAAGTCAAAAGAAA
ACCCCTTTTCTTTTTAAAGCACCCAGTTTCAGGTATTTCTTTATAGCAATGCAAGAAGGGACTAACACAGTTGTATG
TGTATGTGTGTGTTGGGTGATTTCTGGTTGAGTGTCACAAGGTTGTAATATGGTGAGTGTAAGGAAGTATAAGTTTT
AGAAAATTAAGAAGCCAGTTCAGAAAACTAATACTTTTGGAAAATAGTACAAAATCAACTTTACAAGAATATACACA
GAAAGATGTAATACAAGATTTATTTCATTGCAGTAATTTATAAAGTTGGTTTAGTGCCTTGCTTTTGCATGCTGTTT
TAAAAATTACCAAGAATATGACTTCATGTGATTTTGAAATACTCCCAGCAAGATAGGTAGAAAAGGTATTCTTATAA
CTCTTAGACAAAAATTTCGGAAAGTTTAAACGCTTTATCCCAAATCATAAAGCTAATAAATGAAGAATCTGGGATTC
AAACACCATATTTTTTTTACTGTTCATCAGCTAGAAGTTAGAAATGTTAAGCCAAAAACATTAAGTCACTGCTCTGC
CTAATAAATCTTGAGGAAACTAATAAAAAGAATAATACCACTGACTACAGGACAAGGTCTTCCTAAGAGACCTTAAA
TATATTAAGTGATGAAGATGAAACTTCTTTTATTCATAAAAATGTTATTTAGTTATGAGTAGAGCTCTAATTAAACT
TATTTTATATTGTCATCAGTAAAGTTGAGACATAACATATTTATTAATATAATTATAATTTGACCCATAGTGTATTA
AAAGAAGGATGTTAAAAGGAGTTGTTATTAGAGATGATGTTAGGGTTGTTGATGATAATAACAGTAGTCATAACATA
ACAAAGCACTTCATAATTTAAGAAGTGCCTTCAATTACATTGTTACTCTCATGGTAATCTCTGTTTGATATATAGAT
TTGGCGGATTCTATATCACTCTAAGACATAGGTTACTGAGGTGACGGAGGAATTTAGCAAGCGGCTGTCAAATGGAG
GACATGAGCATTGGATTGTGTATGGCAAGGGCTGATGGTCTCTAAGAAAGCCTCTTGGTTTCCACAGGGCAGAAGCC
CTTTGAAGATCATAGCCAAGGATTTAGTAATTGCCTCCCTTTCAGAATACCCTCAAGAGAAAAGCCCACCATAAGAC
ATGGTTCCCTACAGGCAAAACTGCTTTTCCTTAAAATTTACTGTTCCCTGAATATCAGCCTTCTTTGGCTCATTCAA
CATAGTTTTCTTAAGTTTCAGGACAGTGCTGCAGACCAAAAGTTTCAACATTGAGGAAAACAATACTACTTGTGCAG
TGACCCTACCTCAGTCAGGGAGGCAGATGCCTGCCTTTATGTGAGGGAATAAGGAATCAATCATATTTCCAGCACTC
AAGAAAGCCAGTCTAGTGCAGGGAGAGATAGATACATAAACCTCAAAGTTATGATATAGCATAATAGTTTTAAATTT
CCATAATAACTGTATTTTAAAAGTTTTATAGAAACAGAAGAGATGACCTCAGTCTGGAAAAGCCAGCTTGGAGAATG
GCAACCAATATTAAGTGGCAAAAGCTTTGGGATCCCAGGCCTCCAGATGGAGGGTGATAGCATGGGCCAGACAGGTA
GGTTAGGAAAACTTTGCAAAGGACATTACACGGTACACAGACAAGTCTGTGTTTTAGCCTATAAACCACAGTTGCAG
AATGTGTTTGAGCAAAGGCTTTTGGGGATGAGATTTGCACTTTTCAAGATTTAAGTTTGTTTAGGATACTTACGGTT
TGCTGTATACTTCCTGGGTTTTTACATTATAATTACGGTTTGAACTTTAAAGGAAAACTGCAGTTTAGCATACTTGA
AAGAGTGCAACTTCAAGTCATGATTGGAGACAGATATTTAACAGATTTTGTGATCCTGTGATGCTTATTTTCTTCTC
AGACATACCACATGACAATCATTTTTAAACAGTTTATTTCTACTTTAGCATCCATCTGAAGGTGTTGTGTATGTTTT
CTGCTTGAAAATAAAGCAGTGGGCTGGGTGCGGTGGCTCACGCCTGTAATCCCAGCACTTTGGGAGGCCGAGGCAGG
CAGATCACTAGGTCAAGAAATCGAGACCATCCTGGCCAACATGGCGAAACCCCATCTCTACTAAAAATATGAAAATT
AGCCAGGCGTGGTGGTGCATGCCTAGAGTCCCAGCTACTTGGGAGGCTGAGGCAGGAGTATCGCTTGAACCCGAGAG
GCGGAGGTCGCAGTGAGCCAAGATCGTGCCACTGCACTCCAGCCTGGCGACAGAGTGAGACTCTGTCTCAAAAGAAA
TAAAAAAGAAAATAAAGCAGTGAATGCGATTAAGATGGATTTATTATGATCATAAAGTACTCAGGAGTCTTATTTTA
AAAGACAGCATTACTGTAATTAAAAATATAGGGAAGAAACTAATGCTGTTTTGCGTATCATTCTCAGCTCTCTCAAA
ATCAGATATTAAGCTCTTGCTGCCAAAGGAGACTATACTGCACGGTGCTCACCTGCATAAACTTTGAGAGGGTTGAA
TTGTGCCAAGCAATTCTCTCAATACATAAATTAACCAAATATTTGTTGACCTACTGTGTGACAAGTATTATTCCAGG
AAATAAGAGATCCAGCAATGAAACAAGTATGGCTTCTTATAGAGTTCCCAAAAAGGAAATAAAAGGATATACGTATA
GTGATATCCCTGAATTAAATTTCTCTTTTGAAAATAAAAATTCTATCATAAGCTGTAACTGCCAACACTTCAATACT
CATTCAGCAGTTTTCAGGGATTTGTACCTTTTGACTTATGAGAATTTGGAAGTCTAATTGTATCATTGCACTGGAGT
CTTAAAGAAACAGATAAGCGAATGACTTTGCCTGTATCATTGTTGACTGTACTTACAATCAGAAAGGGGCACAGGAC
AGATGCCAGGGAGTAAGTGGACAGCCCATAAATGGAATGGTAAGAAAGAAGAACTATAGTGGATTTGGAAAGTTCCC
TTCAGCATTTTCCCTAGACAATCTTTGGCTGTGTTTGCATGATCAGTATTTCATTCACAGGATATTGAGCTCTTGAT
ATAGTTCTCAAAACCCAAAATGAAATAAGAAGTCTACTCTTTATTTAAATTCAAATTCCAGAGAGTTAAGTAACTTT
CCAGGAGGTAATCTAAATATGGCCTCCTTGTTGGGGGGGGGGGGGGTGTTTGAATTTGCATATAAATAGTCTCACCC
TTAAAGGAAAACCACAGATGGTGGTAATGATGTAGTCATAATGTACATCTCCACAGTGGTGGAACAAAATATCCACA
GTTTTGCTTTCCCCAGTTTCAGTGACCCATGGTCAACTGCTGTCTGAAAATAGGTGACTACAATACAATAAGATATT
TTAAGAGAGAGAAAGAAAGATCACATTCACATGATTTTCATTACAATGTATTGTTATAATTGTTCTATTTTTATTCA
TGATTTTTAATCTCTTAACTGCGCCAAATTTATAAATTAAAATTTATCACAAGTACATATAGTTTATATAGGGCTCA
GTACTATCTGCAGTTTCAGACATCCACTGGGAGTCTTGGAATGTATCCCCTACAGATAAGGGGTAAACCACTGTATC
CTATTTGTGTGAATGCTACAGGTGTTGTGAGCTCATAACAATATGACATCAACACTGAACTAATCCAGGATTTGGTA
GTGAGAGTGATGTATTTGCAAGGAGTGAGACGTGGTGCCTCATCCAAGCAGAGAAATAATTTTGAAATTTGCCTGAC
AATAAAAATCACAATGTGAGGTCTCTCTTTAGAGCTGCAAAGTCCAATTCAGTGCCCCCTAGCCACATAAGATACTG
AGCTCTTAAAATGCGGCTAGTACTAATTGAGATGGGCACTGAGTATAACACACATGCCAGGGTTTGAATACTTAGAA
CCAAAAAGGAAGTAAATGCTCATTTATTGCATGTTAAAATTATGGTTTTATTATAGTTGATTAAATAAAATATATAA
TTAAATTGACTTCATTTTGCTTTTAAAAATGTGGCTATGAAAAATTTCAAATTATATATGTGTGTGATTACATATGT
GTGTTTTCACATATGTAACTGATGTTACATGTGAAATTGATTGTTACATGTGACATGTAAAACACGTTACCTAACAC
GTGCATATGTATGCAACACATATGTAACGTGTTACATATATAACACGTTACATATGTATTGTTACATGTGTGCTTGC
ATTACACACATGCATAATATGAAATTACATGTAATTTCAAATTACATGTGTATATTTTGAAAATTACAAATTACGTA
TTTTGTTATTTTTGCTTTACAAAGTCAAATTTACCCTATTTAATAAAGCATCATGAGTTTTTTATAACTAGTAAACT
TTGAGACTTTTGTAGGAGAATAAATAATGCTTATTATAAAAACTGATTGGAAAAGTGAGCTGGAGCAGGGAGCGGAG
GAAAAAGGACTAGAGATCACCTTTCTTCCCAGCTCCGCTCCTCTCCCAACCTTTTTTCTTTCCATTCTCTCATCCCA
ATTCAAAAGTGCAGAGTTCACAGTTGGTGTGCTGATTTAGAAAACAGATATATAAACAGCCTTAAATTTTCTCCAGG
CTTTTACAATGAAAAGAAGTTCAATATCAAAAGTAACAATATAATCTGTGGAAAGGTATAGGGGGCTATGTTTTTGA
GGTAGAAACTATAGGTGCTCCTGGCCAAGCATGGTGGTTCAAGCCTGTAATCCCAGCACTTTGGGAAGCTGGGGCGA
GAGTATTGCTTGAGCCCAGAAGTTTGAGTCTAGCCTGGCCTACAGGGTGAAACTCCACCTCTACTAAAAATACACAC
ACACACACACACACACACACACACACACACACACACACACAAAAGCCTTGCGTGGTGGCGCTTGCTGATAGTCCCAG
CTACTCAGGAGGCTGAGGCGGGAAGATTGCTTGAACCTGGGAGACAGAGGTTGCAGTGAGCTGAGATAGCACCACTG
CACTCCGACCTGGGTGACAGAGTAAGACTGTCTCAAAAAAAAAAGAAAAGAAAGAAAGTATAGGCACTCCTTATATG
CAGCTGCTCACACCCCTCCTCCTTCACACCCCTCCCCCTTCACACCCCTCCCCCTTCCCCAAAATTTGCAAGGGGAA
AAATGTGTGTAATTGGCAGTATTTAGTGGCGTGCAACCGTGAGTCATCAGACTGCACATCCTCACTTCTGCTAGTGG
CTCAGTACCCAACAGCACTCAGTGAAAACTAACTCATTTCAAAGGTGAAAACAAGTGAGTTTGGCCACCAGGGAGTG
TTCAAAACTGTCAGTGCTGAAGCAAATGTGGAGGGTGTTCTGTAGTTTGTTCAGGTTGATATTTGTGGTCCAACCCC
TAGCTGAACTACTAATTATTAATATCTGTCTTGATGGTGCCTCAGGAGAAAGCTTCTCAAAGGGAATCAATGTTCAA
ATTATAGTAGGTATCTTGGCCATGGAAGTTATTGAATTTTAGCCAATACTTGCTACTCTTTCATTTATAGTGTGAGA
ATGCAGTGTAATGAACCTGACTCTCACTGTCCTGACTTGCCTTTCTCATCGCATTCACAATAAGCACGTCAATACGT
ATACACATTTCATATTTCTAAAGTTTACTTTATTTCCTTATTGTACATCGCTGTGCTGCTGATGGAAGAGAAAAGGA
AAAACACTATTGATTGCAAAACTGTTTTATCTTTGGTGGCTTAGATTTTTTTTGTATGATATGTAACGTCTTGCATA
CCTAAGGCAACACGAAGCTAAATAGATTTGCATATAGCATGTATTTTTTCCAATTAAATGTTTAATTTTGTTCAGAG
TATACTGGGGACATTTTGAATAATGGAGAAAAGTACAAAGAAAATTCATAATTCTACCACCTATCAGCACAGTGAAA
TTTTATGAAGAAACATAATTTTCATGTAAATCATAGTGAACTCACGGTAGGTTTTATTTAATACAGTAATTGGAGAG
CTGGTAGGAAGACAAAACTGGTTCAAAAGAGAATACAAGAAACAAATGCTTCTATAATGAGTGAATTTTTAAAAAAG
TATTCTGGAATAAGATTAGTGAATAAGATACTAAACTCGTTGATACCCTACAGCCTTTGGGGTTATATCCTCTACTG
GGTAAAAAGTCATTTACATCATATCAGTTTTCTAAAATTTGCATTGAACTTCATAGCGTTGTAACATGTGTGGGCCC
AAATTAATAGTAAACAGTAAGAGTTGCTTTACTCTGAAAATATTGAAGCTCTTGTGAGGGTGTGAGGAGTTTGTTAG
AAAACAACGCTACCATTATTTTGAAACACACACGATCATCTTTTGTTTTACTTCTAAGTTTTGGATAATTTTTCTTA
AATTATCTTATTATCTTATCCATTTTCTTAATTTCCTTAACCTTTTAAATGTTTCTCCTAGGCACTTTTATTGATTT
TTGGAATATAGTTGATATGTGCTGAATTTTTATCATCCAGTTTTAATTCTACTGAAAAATCTAAAAGATGTTCATCA
ACTACTATATTTCAAATGCATACATCCCCTTTCATGCTAAAGAAACTGTATGGGAAACACAGTCTGACATTTTCAGG
ACCTGGTATCATTAAAAGTCTTGACACTGTTAAAATTAAACAACGCCTTTTTTAAAATCAAAGGATACAAAAGGGCT
GTGTTGGTCAGAGGATACAAAATTTCAGTTAGATAGGAGACATAAGTTCATGAGATCTTTTGTACGACATAGTGACT
ATAATTAATAATAATATGTTTTCGAAAATTACTAAGAGAGTCGATTTTAAGTGTTCTCACCGCAAAAAAATAGTATG
TGAGGTAATGCATATGTTAATTAGCTCATTTTAGCTAGTCCACATTTTTCAATACAATGTGTTGTATAATACGTGAT
ATATACAACTTATATTTTCCAATTCCAATAAGTAAAAATAAATGTAAATTATTTGAAATAAATAAAATGTGAAGAAC
ATCCACTTTTCATATGAAACCATGAGATATTTTCTGTTAAAAGATTAAATGTCCAATAAATTTTTGATGTTAACAGA
AACAAAAATGTTTAATATTTAAATACATATTTGCATGCTATTGACCCCCTGAAGTTCACTGCTGGGCTAAGTGAACC
AACTATATCTTAAGTCAAAAATGCTGAAATTCTTCCCCAAATCCCAAAGCTCATGAAAACATAAACAGAAAATTTCC
AAATAATTCTACAGGGAAAATAAGACACACTATTTGATCTGATCAAACAACGGGATGATTATGGTTAATAATGAGTT
ACTTGTACATTTAAAAATAACTAAAGGAGTGTGATTGGATTGTTTGTAACACAAAGGAGAAATGCTTGAAGGGATGG
ATACCCCGTTCTCCATGATGTGATTATTACCCATTGCCTGCCTGTGTCAAAACATCTCATGTACCCTACAAATATAT
ACTCCTACGATGTACCCACAAAAATTAAAATAAAAAAGAGAGGGACCCGAAGATAAGCTAATATTTAAGCTCATCAT
ACTTATTAAGATAAGCAATACATACCGAAAGTAATAGCATTTAAAACCAGATGTTGGGGGAGGGTTCTAACTTGTTC
ATTAAAATTCAAAGTCACCTGTCTTGTTTTTTCTTTTGTTTTTGTTTTTTTTTTTTTTTTTTTGAGATGGAGTCTCG
CTCTGTCACCCCAGGCTGGAGTACAGTGGCGCGATCTTGGCTCACTGCAAGCTCTGCCTCCCGGGTTTACGCCATTC
TCCTGCCTCAGCCTCCCGAGTAGCTGGTACTACAGGCGCTGGCTACCACGCCCCGCTAATTTTTTTGTATTTTTAGT
AGAGACGGGGTTTCACCGTGTTAGCCAGGATGGTCTCGATCTCCTGACCTCGTGATCTGCCCACCTTGGCCTCCCAA
AGTGCTGGGATTACAGGCGTGAGCCACCGTGCCAGGCCACCTGTCTTGTTTTATCATGATCCCGAGAGTATATATGT
ATGTGTACAGCTCATCTAAACCCTTTTTCTTTCAACATGATCAATAGATTGAACATTGGAGATATTTTATAAGAAAT
AATGAAGACAACTCAATCAGCACATATATATATTAAATGTGGAATCTATAATGATTGCGAAGCCTGAAGCAAACTAA
ATATTCAGTAATAGGTTCTTTTTTTCCATGGTATATCCATTTGAATATATAACATAAATGCCTTACATTTGTTTTAA
CTATTTAAGGTTTATGTTGTTAGTGTGATGAAATGGCTGGCAAAAGTCAGAAACTCAGGAAAGTTTCAGGCTTATAT
CTGGAGCCTGGTTTTCTTTCTTCAAGGTAGAACCTCTGTGAAGTGAAAAATTTTTTTTATATCTGGAGCAATAATGT
AGAAGCTTAAATGTATTATCCAAGTTGTCATAAGCCTATTATTTCTTTACATTACTGAAGTGAAAGACAGCATTAAT
GGCTAAATGCCATACTTGGCTATAATTTATATTGTTTAGGACTGGAAATGAGCCTGAAATGTACATTTTTTTCCAAA
ATAGTTCATGTAATATTTGAAACCTGACAAGTAACCTGATGATTTCATGGAATACCATCAAATATAAATGTGAAGTT
TTAAAGACACAGGGAAATACTCAGAATAAACCCCCTAACCACAGGCCAGCAGAAGAACTAGACTTGAGAAAATGAAT
GGGAAGATAGATAGTAACAAATGACTTCTTTGGCAGCCTTATATATGCTTAGTCTTATAGACTGTTTTATGGATGCT
CTGCACTCTATTTCCAGCAAGTATGGCATTTGGAACAGGACCACACGAGACAAACTATGAGTTCACATTTCCCACAA
CTGCACAGATAGAAAGAGGGAACAACAGAATACTCCCTTTCTTCTTGAAACAATAACTTCTGTTGAAGCTCACTGGC
TTCTTTTCAGCTGTTTCTGCTAGCTCCTCCTCCGCCTCTTGACCTCTAAGGCAATGCTCTTCAAAATTTCAAGACTG
CTTTCTAATTGAAACAAAACTTATAAGCACATTTCTTCCCACAAAATGTACATTTATTTGTAAATCATATATGAATA
TGACTAAGCATGTAAACGTATGTGAAAATAGAAATCAATAAATATAAATGCAAACACAAATAGAAGCATTCACAGTT
TTCTTTTGTGTCCCAGTGAGTTGTTCCAAATTCCTCGGAGGTAGGTATGTCACAGTTTGAGACTATACCTTCAATCC
TAGGGTTTCTGGTTTCGCTCTCCTCCTAGGTGATAGCATCCATTTCTACGGACTTAACTGCCATCTTTAGTTGAATA
ACTCCTCTATCTTTCCATCCCATATTTCTCTTGATTCCAAACCTGCTTGTTCACCTGAGCATATGACACAATTCATT
GGCTGCCGCACATGCAGCTTTGACATTTTATTTAAAATCTTTCCCCTTCCCCAGCCCTCATCTATTTCACAGTAGTA
TCTTCTTCTTATCTACTTGATTGGTAAGCAGAGTCCACATGATTCCATCATTTATCTCCCATTTTATATCTAATCTA
TAAGCAAGTAATGCAATGCAACTTCTGTCTCCAAAAATTTATTTTGAATTTGCCTTCTCTTCCTCTGCATCTCCCCC
ATCTTAGGCCAGGTCACCTCTGCCCTCTTGCCAGATTAGGTCACATTCTCTTACTACTGTTGTTATTCTCTTCCTAT
TCAATCCTACACCGCAGCAAAATGGATCTTCTCAAAATGTCAGCTAGATAAAGGCATTTCTGTGCTTAAGGCCCTCA
TGGATTTATCTTATTAGGATGAACACCCAACTCTTTATTATGGCTTAGAATACAATGAATTACAACACATAATGAAT
ATATTATATTTCTATCTTTACCATTTTCTTCTTAAGTCAACCTTTCTCAATCCATATAGGATAATCATATTAGTGCT
TCCTCACTTTCTAAAACATCTCAGGGCCTTTGCACGTGTTTCTCTGTTCTTAGACCCAGAATGCTCTTCCTTTTCTC
TTTGTGTAGCTAGGTGCTTCTTTCCATTTACGTATCACATGAAATGCAGTCATTCCCTCCTCCTTCCCTCACTACCT
CACAAAAAGTTGATGCCTCTGTTAAACCATGAATGGAATTTTACTCGGCAGTGAATAGAGGAAAAACCAATGGTAAA
AGCAACCATATGAATGAATGAATGTCAAAAATATTATGCTGAGCCAAAAGTCATAGACACAAATATGGGTATTTACA
TGAAGTTAAAGCACAGCAAAACTCAATTACGGTAATAGAATTAAGAAAGTGGTTACCTCTGGGTGAGGGTTGGAATT
GAGTGGACAGAGGCATTAGTGACTTTTTCGGGGTAATGGAAATGTTGTCTATTTTGTTCAGGTGGTGAATACATAGA
TACATTCAATTGTCAAAACACATCCATCCAAACACTTAGACTTTTGCACTTTATTATATGCAAATTATGCCTCAACT
GAAAAAAGTTTGTTTTCAAAATTATATCAACAGTTGAAATTCTTTTAAAGATTTGATTCAAATGAGATTAATTCTGT
ATCCATCATTGATGTATGATAGTTTTGTATGTAGTTAAGGTTATTGGAGATAATTGAAAGTTATACTCACAAGAAGG
CTGCATAATATGAAGTTTATCTGCCTTGATCTTTAATAGCTTTCGCGATTTCAACTTCTTCACAGCTCTGTAAGAAG
GCAGTGTGGCATGTTGAAGCAAGCATGTGTTTTAGAGTAACACAGAGCTGGTATACAACCCCATGTCTACCAATTAT
CAATGATGTGGGTATGTTGCTGGATCTCAATAATCTTCCACTGTGAAATGGAATGTAACACCTGACTCACAACGCAA
AGGTATTTACCTTATGTAATATAATTCCTGCGATCCTGGGACCTCCCTTAATCCCATCCACAGATGCCAGGTTAAAG
ACCCCATCACAGACTAGAACAAGTTGGGATGTCAAAATGAATAAATATTAATCGAAGGGCCTATTGTGATTGAACAC
CACGCAGTAGGCACTCTCTAATACCTACCGTCTCCCTCCTTTTTGGGGGAAACATTCTAAATGTGCAAAAAATAAAG
GGTTATTTGCTTTCTGGCACTTGGGATCGATTTATTGAGGATATGTTAGCAGAACAGCAAAGGTGAAACACTAAAAG
CACCATCAATACACAGGCAGAGGTGAAGCCATAAAGCCTTTATTTTTTAAATTAATGCACAATATATAAGAGGTATG
TTAGAATGAACGTCCAATCCCTGAAAGGATATACGAAAGACATTCATAAAATTACATGGGCATGTTTTCTTAATGTT
CAAAATATTGTTTTAATTAGTGTATTATGAGTTTATTCATGTGTCTGTGTGTTGTGTTATATTAATCTTTTCTTGCA
TTGCTATAAAGAAATACCTGAGACTGGGTAATGGATGAGAAAAGACACTTACTTGGCTCACAGTTCTGCAGGCTGTA
CCGGAAGCATAGCAGCATCTCCTTCTGTGGAGGCTTCGGGAAGCTTCCAGTCGTGGCAGAAGGCAGAACGGGAGCAG
GCACTTCACCTGGCTAGAGCAGGAGCAAGAGAGACAGAATGAAGTACCACACACGTGTAAACAGCCAGATCTCAGAG
AACTCACTCATCATCATGAGGATGGCACCAAGAGGATGGTGTTAAACCATTCATGAGAAATCCACACACATGATCCA
GTCACCTCCCACCAGGCCCCACCTCCAACACTGGGAATTACATTTCAAGATGAGATTTGGGCGGGGACACATATCCA
AATGATATCCATGTTTAATCAGAAAAATAAAAGTTAACAGTAACAGTGATTTTACTTTGTAGACCTTTGCTAATGGC
TGAAATCTAGCTCCATTCCGAGAACAGCCTGCGGTACACATTTTGAAAGATAGTTGATTAATATGAAAGAAGCCTTA
TCTGTAGTCCTTAAGGCCATTATGGTTTACATATATGAGTAAATATTCCAAAGTAGCCATGCCAGTTAACATATATC
CAGAGTCTAAAGGCCACTGGGCGACAAAAGTAAAAGATACATAGCAATTGTTACTTTATATCACAGTAATTCTTGTA
TATTTTAAATGGATATTTGCATTTGAGGATATCCACTTAAGAGTTAGGTACATGGCTCTTACATTTAAGTAACATTT
ACTTAAATTTCTGGCTGCAGCAATTCCACATAGGTAGAAATGAAGTCTGAATTGAGTTGGGGGTCTTTGCAGTGCTC
TCTCTGTTCATTGGCTATTTTGACAATGCTGAGAGATGTGGTTAGCCATTCTTTTTCATTTCATATTGGCAACCTAG
AGAGCAATTAAGCCTTCTCCCCTTAACTAGATGTATGTTTTACTCATTTCTGGATCTTTATGGCTGACTTTGAATCC
TAGCCTGTGGTAGAAAGCATGGTGTCAGAAGGAACTATGAGTTAAGACTATGCATACTTGGCTTTGAGTCTTGGGTA
TCATACCTCCCTCATAGAGTGAAGGAACCAGGGATTCTTCTTGAGGCCCAGACCCGGCATCCATGTTAAGAATACCT
GTGCAATTTTGCTTCCTGATATTTAAGGTGAAAATGCATGTTTGGGTCATTGTGAGGATTATGTGAGATGTTACTTT
TAAATATAGGCCCCCTTATTATATGCTCTCATAGTTTCAGGCAACACTTGTCGTATTTGTAACCTCAGTTTTAACTG
TAATGTTTCCATCAATGTCCCTCTTACCTGGTACAGGGGCTCTTCATATTCTTGGATTACAAATCTGTGAATGCAAC
CATGCATCAAAAATATTCAGAAAAACAATGAATGCCTACCTCTGTACTGATGATTTATAGGTGTTTTTCTTGTCATT
ATTCCCTAAACAGTACAATGTAATAAGTATTTATATAGCATTTACATTGTATTAAGTATTATAAGTAATCTAGAGAT
GTTTTAAAGTATATAGGAGGATGTGTGTAGGTTGTATGGAAATAGTATGTCATTTTATATGTCACTTGAACATTTGT
GGATTTGCTATCCGTGGGGATCCTGGAACCAATCCCCCATGGATACTGAGGGACAATTGTATTATAAGCAGCAAGAG
GGAAAGGAATCTGTCTATTTTGCCCAAAATCGTGTTCCCGGGACCTAGCATAGCTCCTGGCAAAGAGTATACAACAA
ATATGCATTGAGGAGAGAACAGAGGGAACCATTATCCCCTTATTCTCGCTGTTCCTTCATGTAATGAATAAACAGTC
AAATCTTACAAGAGATTTTAAACCAGTCAGAGAAAAGTTGGAAGTTAGTTAGTTGTTCATACATTGAGAAGCCTCGA
CGCTGTGTCATCTAGGTAATGAAAGATCTAGGGAAGTTTAGCAGGGAGAAGAAGAGAGATGATAGTTGTCTTCAAAT
GTTTGAAGGACTGTTACGGACACAAAAATTTAAACTTGTGCTGAATAATTCCAAGAGGTACACAGTCTCTCGATAGA
AGCTAAAGTGGGGGGTGACATTTGACTCAACAAAAAGCCATCTAAATATCAGAACTTTCAAAAGCAGGAACTGGTGC
CTCAATTAATAGTGTGTTTTCTAGCACTTATGATACCTGATCATAGGCAAGATAATGAAAAATTGGGACCTGGGAGT
TATACATGGGAATTTGTTTATCAGTTGGGTGATTAGGAGAGGTGGCCTTAAAGTCCTGTTGTGTTCTAAGAGTCTGT
GATTCTGAGTCTTATTTCCCAACAAGAGAGGTACAGAGCAGAAGATGGGATTGGGAGAAATAGGATAAAGATACCAG
GAAATCCTAAAGGTAAGAAAAGGAAGGCAGACCTGAAGCTAACTCTATACTTCAGGTGCTTGCCTAGAGCCAGCCCT
ACCTACTTAGAGAATGTTGAAGAGCCAGTTAAAACATCTTTAACACGGATGTAAAACAAAACTATCAAAACCTGAAG
ATTTCGAATGTTCTAACCTACTCGTCAGTTGGGCTTTTTTCACAAATACTTCAGTAAATAGGCATAAATTTATTTTT
TAATGATAGAAAATATCTCTTAAAGAACTTATAACTGTGGATAAAAGCACCACCATAAAAATCTTGTGGTGAAATAT
ATATATATATATATATATATATATATATATAAAATTTTAAATATGGTTAGCTAGAATATGACGACAATGTTTATGAA
ACACAGAGACTCTTGACAAGTCCCATGTATACACTATAAAACTTTAAGTTATCCACTATTCACTCACTAAGCTTATA
CTTAATGAGTGTCTGCTGTGTCACTTATTGCGGAAGGCACAGGCGGTATAGCATTGCACAAAACATATGTGGTCTCT
GATGGAGTTTTTCAGTCTAGTGGTGAAAGCAGTGAATGGGTGTACAGATGTTAAATAATTGTACAATTAGTTGCATG
TGTAAACGTCAAAGTTCAGAAGATGACAATTGATCTACGGCAATGTTTCTCAATCTCTGACGTTTTGAGCCAAATAC
ATCTTTGTTGTGGTGGACTGCCCTGTCCACTATAGGATGTTTGGCATCACAACTGACCTCTGCCCATTAGATGCCAA
TAGTACTCTCTTCTTTAATCACAAATTTGTCCCAGACATTTCCAAATGTCCCTTGGGGAGCAAAATCATCCCTAGTT
GAAAATCACTGGTCTAGGGGGAGGTCTTTATGAGGAAGTAACATCTAAGAAAGCTGGTATGTTTACATATAGCTACA
GTCTATTACACATGTATACATATGTAACAAGCCTGCATGTTGTGCACATGTACCCTAGAACTTAAAGTATAATAAAA
AAAATGTAACAAAACAATACAGTATGATAAGTGCTATGGGACCAAAGATGAAAGGGTTCTACTGCACAGTTATGAAC
TCATAGTTAGGCTTTTGGGGTCAAAATTTTGCTGAAGATATTTGCCACCCACGTGACCTTTGGCAGGTGACTTAGCT
TATTCATGCCTCAGTTTTATCCAATGTGAAATGGGGCTGGAAAGTCCCATGTACTTCCTAATAACTTTGCGGAAATA
ATATGTGGTTATATAGGAAAAAAAAAAAAATCCTAGAAGTATGCCTGCTGCGTAGTAAAAGGAAGGAGAAGGATAAA
GAGAAATCTGCATTTTTTCTTCTGTAATGGGGCAGATAGTAAATATTTTAAGTTTTGTGGCCCAAATAGTCTCTGTC
ACATTTACTTGATTCTGCAGTTGTGGCATTGGAAGCAGCTATGGACAATACTTAAATTAGTAGGTGTGCCTGTGCTT
TCAATAAAATTTTATAAATACAAAGTTTGCAAAACAAAGTTGTTTTTTTTTTTTTTGTAGTTTGCTGACACCCTAGT
AAAGAAGCACCATTGTCAACGTTAAAAATTATCAAATTTTTATTTTTCAAAGTTTTCAAATTTGCTTTGCTTGGTCT
AGCTCATGAAATAAGTCAAAAGTAGCAAGACCTCCACCTCTAAAATAATAATAGTAATGATAACCTCAAAAGGAAAG
AAGAAATATTTTTAAAGAAGAAAAATTATTGTTAAATAGGATTATTGTGCAGAGAAAACCTAGGAGACTCAATTTTA
AAATCTGTGAAATAATTTTAAAAATACTTTATGAATAGATACATAATAGCTTTTATTCATATTAATGACTATAAATG
CAAATGGAAATATTTCATTCACACTGATGACAATGTATAAATTAAGGAGGAATAAAAATTGTAGACCCTATAGGTGA
AAAGCATAAAAATATACATAAGAAAAAGCAAAAATTGACTACGTAGGATTGTTTTAGGATTTAAGATTTATTGTCAT
TAAACTTGCAATACCAGCCAAGTTAACATTTGAATTTAATACAGTTATAATCAGAATGCTTTTGATGTGTTTGGGGG
CAATATAATTTCAAAGGAAATAGGCAATGATGTAATTTAAAGTTTATATAGAAGGAAATTGTGTGCGTGTATGTGTG
TGTATAAATTGGAAACAATTTTATTAATAAGCATATTATGGCAGCAACATACACTTCCAGATTTCTACTATACTTTG
AAGTAATTGTGATCAAAACCACAGTGTGCTGGCATAAGGCTAGAGAAATGGGTTAGTGGTTTACAAGTGAGAGTCCA
GGAAAACATCCAAATAAGATTGGATATTTTAGTTCTGTGTGGATAGCCTATTTCACTTAATAAATAGTGTCTCGTAA
TTGACTATTCATGTACCTATAAGTTTAACTATAGACCAAAAAAACGCCCTACTAGATTAAGGAGCTAACTAGAAATA
TAAATTCATATAAACAATAAAGGAAAGTGTAGGACTTTATAAGCTTCATGGGAGACAGATTTTTGGTAAGTCAGGAA
GCCTGGAAGACTTAAAACATAAAATTGGCAGACTGAATTAACTGATAGTTTAAAGCTTCCATAGAGCAAAATAAATC
ATAAACCAAGTTTTAAAATATATAATGGATTTAGAGAAGGTATTTACAAAAATATATGACTAATGGAGGTTAATAAT
AACAATATGTAAGAAGGATATGAAATGGCATTTTACTATAAAGGTCAAACAAATGACCTATAAGCATAATAAATCAT
ATTAATCTCCACTAGTAATAACTACACACATCTACATAATATAGATGTTACGCCTGCATTTGATTTACTTTATCTGT
CTTTTGGCAGAACTATTTGTCACCAGATAAAAAATTCTATATCATTACCAGAAAGGTATATTATTATAATGTTTATT
ATGTTGCAGTTGTAAAAGAAATAACAGCTTTTCAATTGTTTACAAATCCTATAGAACATTTACTGAAATACATTTAC
ATTTTGTGGCAAACTTGGATTTAAATACCGTGTTCGTGCTTTGTTTTATGCCGTTTTCCCATCTTTTCTCCAGGAAT
TTGATTGTGCTTCATTGAAAGCTAAAAAGAAAAAAAAAATAATTCTGGTTTTGGTTTAAAAAATTAGGTTAGGGGTT
AAAAAGTTGTACGTTGTCTTCTGTAAAAATAAAAAACAAGTTTTCTTTGTTTCTTGGAGGCTTTATATTAAATGGAT
TTTTAATTCATAGACAGCATATTGTGATGAAATTTCCCCATGAGCTTCACATTTTGTTTCAATAGCAGAAACTAACT
TGGTTGCAGTTACTGCCCTTCTGAGAACAGTGTTCTGGAATAATTTTGACATACATATGTATCTCTTTTTAAAACAT
GTGTTAATCTTTTCATAAAGAAAGTTTTCCCAGCTGTGTCACCTGTGACTCCAACTTTCTGGGGGGACAGGGATATG
AGATGTTGGAAGGGAATGGCTTGAAGAAATAAAGTGCAAAAGACGTAATGCTTTCCTGTGGTAGAAATGTATTCAGT
GACCCTGAATGACCTTCCTACTCTTGTCCCTTCATTTTTCCCACAAGTATGGTCTGGGCAATTATAAAAATTGACAT
TTGCAGTGGGCTCTTCTGTAAAAGATGCTCAATCAGAAATGATTTATTTTAGAAAAAGAGATGATATAAACATATAT
ATCCCCTGTCTCGGAAGTGTGAAGGTTGAAAAGCAAGGAGATGATCTTCAAAGTGTCTAAAATATTGATTTGTAACA
TCGTTTTATGAAAGTGCTTCAGATTATTTTTTTTCTTGGATGGCCCCTTATGCTTTGGTCAGTTGATGCTAAAATCT
GAACTTCTTTATTTTAAAAAAAACTTTTAATTTTGAAAAAGGAAGTTCACGGTGCTGTCTAATTCTTTTTAGATAGT
CATTAATGTAAATGTAAGAGTCATTCTGAGAACCACATCTGCTGATATGTTCCGTTAAATTACAAGTTCTATGTGTA
TTTGCTTTGCTTTCATACAATGAATCTTCTTTACTCTCTTCCCCACCTGCCAGAAATTGCCCCACTCAACGTTCATA
AAAGGTCCATTTTCAATCGCTATATTTATTTCAGAAGCAGAGATATCATATATTCAAATTTTAGTTACTTTCCAATA
TCAAGCTAATAACTCACACAAATAAATCAAACTACAGCAAAACAGCAATCTAGCATTCAACAAAACCTCCCCAATGC
ACATATTTCAAGCTGTAGATATGTATCATCCACCATGCTGAAATAATGTACATGTTCAAATCAAATGGAAAACTAGA
ATCAAAATTGTTGATTACTTCTTATCAGGGCATTTTATTATATTTAAGAAAAATACAAATTAAATCATTTTCAGGAA
GCAATCCTTCTGGCTAAGATTTTTTTAGCATAATGCTTAAAGTTAATTGTTGATCTTTATCTATAAATTCAAAGGTG
GACTAAAAATGCAGAATCAATCAGGTAGTCCATTTTGCATCAGGTGAAATATATAAAGCATAAAACAGCGAGTTACA
TTTCCTAACAAAATTGAATTACAGTGAGTAAAAGTGACAGGACAAATGCATTAAGAAAAGATGGACTGAAATGGATA
GAGTAGAATATATGCATCTATAAAACACAGTCATATATAATACACTCATTTTTTTTCTTACGAGTGTGAGATTAATG
GAAGAAAACAACAATAATAACAAAACCAGTGTGATGTGTCAGATTTCACCTTTTAATTAAAAAATTATTCACTTCAG
AGGGGAATTTTCTTTCTTGGGTTAGCTCAATCATGTCAGATCTTGTTCATTTAAAAGGTCAGTTTACTTGCCTTCTG
AGGTTTTTGTTTGGGAAAAAGAAAAGAAAATAGATTTTCATTGGTATCCTGGGTAGAATTAATTGTTTATCATTCAT
TTTTAAGATCTCCGAGAGGCAGAAAAAGGGGAACTGTGCAACCCTTTTGTCCTTCTGGATCTCAAAATGAAGGGATA
CATTCTGCTACATGAAATGTGGAATTAAGACCATGATGCAACATGATAAACAACACAAATTTGGGGGTGTCTCTGTG
CTATACATTATTGAATTTTTCCATGCTATACACTTTTTGGATGTGTCTGTGCTATTTATTCAGTTTTTTTAAATAAA
AGTTTTTGTAGACTAAATTGCCCTCTCTACTTTGCATCGTTTTTGAACAAAGGATTTTCAAGACTGATAAGCTCAAA
TGTATCATTTATTGTATTCAAGTAGCATTCAATTTTTCTTTAGAAGTATAATTTGTAGATATTTTAACACAGAAAAC
TTGCAACACTGCTCATGATAGGCACTTATTATATATTTTTTGAAAGACTATATGGATAATGATTCTAACTTTGACTT
TTCCTGTTTTGCCTTCACTTTAGAATTAAGCAGAGAATCAAATCCATATTCCTGGGGGCGATGCTTGGACAACAGTA
TCTCTTTAAAGATCTTTGTGTGAGTCGAAGGTGCAGCCAGACTGGGAGTTATTGTGAAGAAACAGATTCAGGAAGGT
TGAGAAACTTGCCTAAGGCTAATCAGATAGTTACTGGCAATGTTGTTTCTAAATCACTGTTTGGCTCCCTCATTCAA
TGAATCTACACTATGTGGGACTGCCTCTTGCTCCTGACATCTTTTGCTGCTGAAATAAATGAACTCAAAGCCTAGAA
GGTAGAAAAGAGGGAGTTCAGAATTATATTCAGGCACAAATACCAATAAGGCTATTGCCCCCAGAACTGCAACTTCT
CTTGGTTTAACAGATAACTATTTAGCTGTGAGGTACAACTGAGGAAGTGGACACACAAGTTATCAGGAGATTCTGAT
GTGCCAGTTTATATTTCTTGTCACAGGTAATGATTCGAAATTTCTTAAAACAGCTGTCCTCACAGTGGAGTAACCTG
GGAGTACATGAAGGCATTCCAAGGAGTAGGCACAGATAGTTTTAAGGGAATTTATTTCTAGATCTTCTACTTTATTT
TGTACTCTTCCTGAAAACTGAATTGCCTGAAAAAAAAAAAAAAAAAAAAAAGACATCTGTAGTCAAGACCTCAGGCT
GTTTCTCCTTTCTAACCACTTGCCTTTTCTAACCACTTCTCCCAATTTAAGAAAAAAAGCCTTATATTTCATCCAAC
TCTGATCTTACTAAGGCTTCAAACAAAAGAAGCATGAATGACTTTCATGACAGGGCAACATAGCTTTTTGCAAGAAG
AGTGGTTGCTAACTCTTTGCTTTCAACTGAACCCGAAGAGAAGACCTGATAAGTTGTCAGCCGATAGATCATTAAAA
ATACGTTTTGGTAAGCAATCATCATGTACTTTTAGCATATGCCATAGCAGGAGCACAAATGATTAAGCAATGCTACT
ATAATACAATTCCTTCCGTTTCTTTCTACTCACCTATTTGAATAAGATTTTTCATCATTTACATCTATACAGACAAA
AATTAGGGATAGAATTGATGCTGAAGCCTTTCCAATTGTAGAATTAATTTATATTCTTCTGAAGGTGTATAAATTGT
TAAATACCCATCCATCTTATTAAGAGATGTATTTTCAATAAAATTTTATTTTTATGTTTATCAAATTTTATAATATA
CATATATTGTTTTGGTCAATTGCACGTTAATAATTGTAACAATACCTCAATTGAAAAGGTTTGTTTTTTACATTTAG
GACTTACAGTAACAGAAAAAAAACACTCATTGTGTATACATACTGTTTAAGAAAAGTATACTAGGTGATCAATAAGA
TTTTTTCAGGCATAAACATATATCTTAGTTTTAAGATATCGATATTTACAATGTCCCTCAAATTATATTATTTTCAG
TCATTTAAGAATGAAAAGTACATTTCGAATGCGGATTTTAAATCTGCAAGGGTTGACTCATTTTTCAAGAGTCTTTT
TAGGGGATACAGAAGCAAGAATGTTTGGAGTTCCCTGATCAGTATCTTTAAGAGAAGGTATTTGTTGGTAGTTCCTA
GCAAATTCCAACAGCCTGATGCTACTTAAAAGATAATAGTAATTATTTTAAATAATGCTTCTGATAAAAAACATTCA
TGCACACTCAGTTTAAAAAGATATTTAAACATTTGTAGTTGTAGTTTGGGAACTCATGATACAAGTACAGTCTGTAA
ATGAAGCTCTTAGTTTGCAAATATCAGAGATAAGCTATTAAAATGCAGAAATTGAAATTGCCCTGATATATGCATAA
ATTAGTGTCATCTCCATCTTGTCAGTTAGAGTATTTTTTAGATTCTCTCTATGTATACATACATATATATATATATA
TATTTATATATATATATATATTTGTGTAGCTGTGCATGTGTGTATTTGGACTAATGGGTCAAAGGACAGTACTAACC
CAATTCAATAATTAAAGAAAACATAATTTTGAGAATTAGCTTTATGGTAATTGTTTGACTTAAATGAGTAGATCAGA
GAAGAATAAGGGCTTTCCCTTATTTAAACAAGCTTCATTTTTTTATCCAAACATTTACTTAGCTGATTAAGCTTCAC
TTGTTTATTTTCTTCAAAGCATTCATTCAGGTGGGTACTGAGTAAACTGAAATATCACACCAGGGAACTTCAACACC
ATCCAAGTCTTAAAGGCTTCACTTGTTCACAGTTGGCATTTAGTGAATGTCTAGGCTACTGATAATATTGTGAGTAA
GTTGGCAGGGATCATAAGAAATGATAAAATACAGTTCTTGAAAATGTTATGGTTTGAGGAAAAGATCTATGTTTGGA
ATTAGACTGACTTGGATTCAAACTCTGGCTGTACCTTTGGGACAAGGTGTTCAGAAACTCTAGCCTATGTTTTTTTT
CTGCAAAATGATCCTCTTTTCCAGGATTCCTGTAGAGATTCAAAGATATGTGAATGTTTAGAAAAAGAATAGACTTT
TGATCATTGTTAATTCCCTTACTTTCCCCAATTAGACTTGTAAGACTGGGAAGAAAGCTACACAAAAGATTGAACAA
ATTATAGCTGACAGACCATAGCAAAAGATACAGGGCAAAACTTAAAGGGGAAAACTACACATTAAATTATTTTAAAC
CATTAAATAGCACTAACTTTTGTCAGATATTACAACCAAACACCACTCAAATTAAAGTAAACTGAATAAAATGCCTG
TTTTTTTCTGTTTACTGATGTTTTCATTTGCTTCATTCATTTATTGGAAGATATAAAATGTGTTAGACACTGTTAGG
TGCTGAGTGTATAAAAAAATCTTATTAATACAATTTAAACACGCACACACATATATATGGTTATAACAATTGATGCC
ATGTATGTACTGTTTATATGCCTATACATTATTCCACAGACCTGGGGGGAGGGGGATGTAGAGTCTTACCAGAACCA
TAGGAATCTTCTCACATCAACATTTCCTTTTGAAGTTTGTTCATGAGGCACCATCCAGATAATACTACCATCTGCAA
TGTGGCTTGAGAAGATGTTAGATTTTTTTATTACACATAATAAGGCTGTAAAGTATTTCTGTATTTAGGTAGAGGTA
TGTAATACAATATGTATATAAAATTACATATCCAATAAAATCTGGTGTTAAATAAGGACTAGCTTCTATGATAATAT
AGTCTAAAGGCTTTTCATTTGGTGTTATAGAAATTATGTGAAATATGTTTCCTGGAGTAGAATTATTCGCATTTCAG
CTCTCTGACAGTGGAAGAAAAGCTAGAGGGAGAGGTGAACAAGAGAGGGAGCATAATGGACAAAGCTTTGCTGGAAG
CCAAACCACCACTTCATATGTCAAATCTGACAGGCCTCCCATTTTAGGTGTGCTGTCATTGAAGCTTTCAGCTGCAC
CTTGCCTGTGGCTAGGCTATTTTCAAAGATTAAAATGCGAAACTGGAAATTAAATGCAACTTAATTCCCAATTTAAA
TTTCCATTATTTTTGAAAAGTAAAAGATTAAAAGAAATGTATAATTGCAATTCTGGTGGAAGAGGTAATTATAGGAA
AGGTGGGATGTATTTCAAGTGGGGGATATAGCTTACTGCAGCAGAGAGGAATCTAAGCTATCATTCTTTTGAAATTG
GTCTGGAAATATGTTTTCACATGGAAAATATACTATATTTTTAGGAATTTCCTTGTCATATTACTGTATCCTTTTCT
GTTAGAATATAAATTCTGAATTCCCTATTCCACTGTAGATCTGCCTCCGATTATATTAGCTCTTCTGAAGTTATCAA
AAAATAATGAGATATACAATATTCCATATATGTCAAAGCAATTATTTTTAGGTTAAGTAATAAACCAATGACCTTTA
ACCCGGTAATATTCTGGGTTGTTCATAAAAAAACTATATTCAGGTAATAATGTCTTTCCACTTAAGCAACTGAAAAA
ATACACAATACTTAACATTTGGTTAATTAAATACCTACTCCAGACAAAAGGATTTTCTGTTTTCAAGTTATCTTAGC
AAGCTGAGCAGGAAGCAATGATATATCCAATCAGAATATCCATGGAAGCTCTGCTACAGTTTCAAAAAGTTCTCATC
AGGCAGCTTTTAAAATGCCTACTCTGAAAATGGTCCAGGTTAAAGAACAACAGCTTCCTCGTCAGATAGCAGTATTG
CTTGGCCATGTTTCTTCCTAGCACAAAAAAGTACCTGCTCTTCTCTGAGTACCTACATTCTAAGGACTATGGCTTAC
ATAAAACAGCATGGGTTGGGGCAATTTCCAGCACACTGCTCACTCTCGAAAACGTATGATGCAGGTGAGAGTAATGT
TTTTGTTTGAATCTGCTTTCACTCGTGGAAGATGAAACTACTTGCAAAGATCTGTACTTTAGCTATTATGAGTAACA
AAAGACTCCTAAAATATTGCACACATTGTGGGGATGGAGAACCATCATCCTGGGATTTGATGGATCCTATGGTTTGG
CTTTGTGTCCCCACCCAAATCTCATTTTGAATTGTAATCCCCACAATCCCCACATGTCAAGGGAGAGAGACCAGGTG
GAGGTAACTGAATCATGGGAGCAATTTCTCCCATGCTGTTCTCCTGATAGTGAGTGAGTTCTCACAAGATCTGATTG
TTTTATAAGGGGCTCTTCCTGCTTCACTGGGCACTTCTTCCTGCCACCTGTGAAGAAGGTGGCTTGCTCCTTCTCAC
CTTATGCCACGATGGTAAGTTTCCTGAGGCCTCCCCAGCCATGCTGAACTGTGTGTCAATTAAACCTCTTTCTTTTA
TAAATTACCCAGTCTCAGGCAGTTCTTTATAGCAGTATGAAAATGGACTAATAGAGACGTGTCTCTCAGAAGTCACA
GTGATGCTTGAACGGATCCAGAGCTCCTTCTTCAGGAAGGTCCCAACTCATTCTGAAGGGTCTCTCCAAGCCCACCT
CTCTCTGTAAATGGGAAAGGTTTTACTTTGAGCACTAAAACCTGCCAGAATTCTCAATTTTCCTAACAGTGTGTTAA
TAAACACCTACTCATTTAGTATCCAAACCAGGTCTGTATTTCTCAATTAGAGCTCACCAGGCTTTCATCATAAAGTA
GAGCTTCAAATTGTCTGCAATCCCACTCCTATCAAAAACCTAGAAGGAGGTAATATTTCAGAGTAATACTATAACCA
GATGACCACATCTAAGAAACTGCTGACCCTACGATGTAACCTTCTGTCCATTTTTCCCTTTGGAAAGTCTAGGATCT
TTTCTTATACCAGCAAGTTACAAGCCTGGACTACACTAACTTGCTTTCCGCAGAAGAAAACACCATGAGTTCTGTTT
TCATATTAAGCACTTAGTCTCCATCAGACATCAATCGAGAAAAAATCATTAAAAATCACATTTTATATTTGATGTAT
ATTTCTCAATAATCCTATGTATTAGTTCATTTTCCTACTGCTATGAAGAAATACCCAAGACTGGGTAATTTATAAGT
AAAAAGAGGCTTAATGGACTCACAGTCTCACATGACTAGGGAGGCCTCACAATCATGGTGGAAGGTGAAGGGGTAGC
AAAGGCATGGCTTACATGGTGGCAGGCAAGAGCGTGTGCAGGAAAATTGCCCTTTATAAAACCATCAGATCTCCTGA
GACTTATTCACTGCCATAAGGACAGCACAAGTATTTAGCTCCCTCAGCACAGAACCATCCCCGTGATTCAATTACCT
CCCACCAGGTCACTCCCATGACACATGGGGATTATGGGAGCTACAATTCAAGATGAGATTTGGATGGGGACACAGCC
AAACCATATCATCCTATTTGGATGATCAATATTATCAAGGTATGCTCCCCTGAGGGGGCGTCCTTTTTACCATTTAA
CTCCAGGACAAAAGTTTATTTCTTTGTAAGGACAGTGTTTATTTCTTATGGTCCTATTTTCTCCTAAGATCCAGACA
CCAAAATGGCCATCTATCATTGACTTAACTCCTGAATTTTGCTTAGAGTAACAGATTTAGTGAATCTAAATATTTTC
TGGCTGTGGAATGTTAATTTATACATGTTCAAGTTACCTTTGATTCATGTGACAGTTTGTGCCAAAACACACTCATT
ATCAGAACTCAGATCATTATGTTGGCTCTTGTTTTCGTTACTAAAGGAAGAAAAACAGTTTCTCAAAAAGAAAATTC
TGATACCTAGGAAGACCATTATACCTCACTCTTTTCTTTATCTCATCACCACATCCAATATTATAAAAGAACTTACA
AAGTAAAAAGAAAGGTGTTCTGTAGATGTAGCGCCTGGCTTGTATGGTAGCTTAAATGAACACAGCTAAAAATATTT
TATGGCTAGTGTCCAAAACAGTCTGGCACCAGACAAAATAAGAATATTTAAAATTATATTTTAGAGTTACTTTAAGA
GGAAGGGAGAGAGAGATGTAGGCAGGAGGAGGAGGAGCAGGAGGAGAGGGAGAGAGAGAGAGAGAGAGAGAGAGAGA
GAGAGAGAGAGAGAATCTGGGGTTTCTATGGAAGGGCTAAGAATATGTAGAAAACAGTTTACAAAGAAATATGGTCC
AAGAATCGTGTGTACACACACACACACACACACACACACACACACACCCCCTGGAATATTTTTCAGCCTTAAAAAGA
AGAAGATCTGTCATTTGTCCCAACATGGATGGACCTGGAGGACCTTATGCTAAATGAAATAAGCCAGACCAAGAAAG
AAAAATATTGTATGATCTCACTTATATATGGAATCTTTTTTTAAAAAAGGTCAAATATATACAGATAGTGAATTAAA
CAGTGGTTACCAGGGTCAGGGTAGTTGTGAGGAAATGGGGCAATGTAGGTCATAGGATACAAATGATTAAAATATAT
TAATATATTAAAAGATATAATATACATCATGAGGACTACAGTTAATAATAGTGTGTATTCAAGATTTTTGATAAATG
AATAGATTATAGCTGTTCTTGCCACAGAGTGAAAAATGGGTAACTGTGAAATGATAGATATGATAATGTTCTCCACA
ATGGTAACTATTTTACACTATATATATAAATATCTATGCATCTTACACCATTATGTGGTATCCCTTAAATATATACA
ATAAAATTTATTTTACAAACACATATTAGGAATGCATATTCTGATTTTTAACAATAGTTAACCTCATTAATATATTT
CACACTATCATTTCTAGTGTACATGAAAAGTAGTTTATTGACATTAGTTGTAAAAAAAAAAAAAATGGTCTTGAGAC
TTTTGGGTCAGAGAATGTTCTGGCCATAAGGTAGGTTTCTGCTTGCCTACTAGATATCTTAACTTCGATTTCCTGAA
CATCCCATCACTTCAGAATCTCTCAATCCTTTCTAACATCCGCAACATTGTTTTTCTTTCTGCATTTCTTATATTGA
CTGATGGATTTATAATTCACTTTCTCTGAAAAACCCTGCAGTTATCATATATCCCTATCCATTCTGGCTCTTTATTG
CCCAAATCTCTACCAAAATCCTGTCAGCACAGCCTCTGAAATATTTCTCAAAGCATTTATAATCTGGCTCTCATCAA
CATTTTCAACACTCTGTTTTATCATTCCACTATTTTACATCATTTCATTTTCATTTTTACCACAATCACTCATCCAA
CAAATAAGTATTTAGCTCCCTCAGTAATTAGTATTATTATTATTAATTATAACTAGATGCTGAGCATACAGAAGTGA
ACATGACAGACATAATCCCAGCAGGGATGTCAGACTTTATGCAAGTAATCAACCATGATGAATCTCATGAGATTCTG
AGAGAGAGAGAGAGAGATTGAGAGAGAGAGAGAAAGGGGAACCACTGGTGTCCGAGTTAGAAATTTGAATTAGTATC
TGGGTCACCAAAAGCTTCTGTGAAGAAGTGATATAGACTTGGCCACACAAAACTACCGTGAAGGTGGTGGAAATTTT
TCTATGCAGAGTACCACATTTAAAGAGCTAAGCCTGAGAGTGTCAGAGATAAAGGAACAGAAAGAATGTGACAGCAG
ATTATGTTTGGAAGAAAGATGTTCAAGAGACCAAGCTAAAGAGGAGATGGGGCTAGAACCTGGAGGGTCCTTCGGGT
CCTGTTGGGAGTTTTTTCTCTGCCCAGAAGGGCTTTGTCACGTGGTTGTCAGGAAAGAGTCATGATTAGAGCTTTGA
TTCAGAGACTTCTTTCGCTGAAGTGTGGAGAATGGTTCAGAGAGAAGCAAATCTGAATGGACAAAAGAGGTTATTAT
TGTAATCTTGGCAAGAAGCGATGGTGGTCTTGACTAAAATAGTTCTAGTGAGAATGTGACAACAAACCTGAGAAAAA
TACAGGAGACGTAATTGACGGGGGTTAGTGTTAAGTTGAACGATTGCAGAGTTGAATTTGAGGAAAGTGTCATATAT
CATTCCCAGTTTCTGATGTCATACACCTCTGGAGATAACACTGCCATTTCTTTTGAAATGGGAAAATAATAAGTGAT
CAGTAAGTACGTATTGGATAAAATAATGAATGGTTAAATGCATAAGGGGAGAGGAAAAGAGTTGCAGAGAAAGAGAG
TAAACGTATTTTGGATGTGTTAATTTTGAGATACCTTTGAAAAATCCAAGTGAGGGGTTGGGTAGTCAGAGAAATGA
ATGTGGATGTCAGGACGAAAGGTGACCGTGATGAACTGTATGTCTTCCTCTAAGCACGTTATACAGCTTCATGTCAC
AAGTGACTCACTTCATGTCACAAGTGACTCACAAGGTCACTTGTGACAAGCATTTGCCTGGTGCTTCATCCCTAACC
TCCCTTTCTATACTCAGCTAAAATGTCACCTACAATACTTCTTCCTTGACTCCACCGTCCCCACTTTACTGATATGA
ATACATTTTAATAAAATGATATAATAATGCTTAGTTTGTAAACCTAATGTTCCTCAAGTGGTATAATTATCTGATTT
GTATGTGATCATCAACCCAACCATATTAGGAGCACCTTGAAGGTAGAAGATTTAGGTTCATGCTTAACACCACATCT
GGACCACTGTGGATTTAACTTTCTACAATGATTGTATTCATTAATATATTGGGTGCCCACTATATTCCAAGTAATAT
CCTGCACACTACGTACAAGGAAGCATAGGTCCCGTGTGCTCATGAAACTGTAATTTTAGTAAGCAGGGATAGGATAC
AAACTGAGAAAGGAAAACAATTTAGAAAGTGGGAAATATTATGCACAGAATTAATAAAAAAGAGAAAAATCTTGAAA
AAGTCTTCAATACCTCACTTGGAAGGTGATTTTGAAGAAGAACTGATGGACAAACTAGAGTCAGCCATGTAATGATG
TAGGGGCAAAGCATTCCGGGCACAAGGGACAGCTTATGCAAAGACCTTAAAAATGAACTAGCTTTGTATGTTGGAGA
AGGATAAAGAGAACTAAGGTATCTATAAGGTAATTAGGAAGAGGATGAGTTATTTAGTCCCTTAGTCTTTGAAGCAC
ATTATCTCATACTTCAATTGAGTTTATTCTTAGTGTCATTCTTCTGGATGCAATATTTGAGATAAATGTCTTAATGA
ACGTTCACCTCCCTCCGTAGTAATGCCTGAGTGTCACAAAAACTTTTTTTGTTTACATACGTAGCCATCTAATGGAA
ACATAAAATAGGAATCAAAAGTTGAGTTTCATGTACAAAAGGTAAGGACTGTACATGTGGTCATAACAACTTCAAAA
GCACCTGAAGGTAACCTTTAAGGAAGATACAAAGGCTAGGAAATATCTAGGATCCATGAAGACAGACTTACTTAAGG
TCATAGTGTGTCCAGAGTTGGTTCCCGCCGGTGGGTTCGTGGTCTCGCTGACTTCAAGAACGAAGCCACGGACCTCT
GCGGTGACTGTTACAGCTCTTAAAGGTGGCACGAACCCAAACAGCGAGCAGCAGCAAGATTTATTGTGAAGAGCAAA
AGAACAAAGCTTCCACAACGTGGAAGGGGACCCAAGCAGGTTGCCGCTGCTGGCTTGGGGGCCAGCTTTTATTCCC
TTACTGTCCCCTCCCATGTTCCATTTCTGTCCTATCAGAGTGCCCTTTTTTCAATCCTCCCCACGATTGGCTACTTT
TAGAATCCTACTGATTGGTGCATTTTACAGAGCGCTGATTGGTGCGTTTTACAATCCTCTTGTAAGACGGGAAGGTT
CCTGATTGGTGCGTTTTACAATCCTCTTGTAAGACAGAAAAGTTCCCCAAGTCCCCACTCGACCCAGAAAGTCCAGC
TGGCCTCACCTCTCAATAGCATTAAGAATATAGTTTCACGAGCATATATGAATCAAAACTTACATTTGCCAATTTTA
TTTGCTTGTTTATGTGTTTCCAACATGTCTTGTCTTAGGGCCAAATGTTTCCCTAGAGAATAACTATTCCAACTATC
TTAGTTGCTGTATTTTTATGCAACCTTCAACTCTCCATACTAAAATGTCTCCAGAATAGAAAATAAATCTTTTCAAA
GTTTCAAAAGAGGCTCTCTATATATTCCCCTTAAAAGTACCAGGCAGACATATTTCTAGGTTTCTAACATTGCGTGT
TGCCAGGAAGTATATCCAAACCATCACAAGTTATTCATGTAACCAAGCACACTTATTGGAGTGCTTCTGCTTCTGTT
CTTGCTTGAAATTGGAAGCTCCTTCCAGGAAAAAAAAAAAATATCTATAGAAGGGGAAAAAAGTAATTTTACTTTGA
AAATAAAATATACGTGAGCAATAGTTTTATTCTGTTTTTAATTTACCATAGCTTCCAAAGACAACATTGTTTTATAG
TAGGGGTTAGCAAGTGTTTTCTGTAATGTAAACGTAAAGGGCCAGAGAGTAAATATTTTAGGCTTTGTTTTCTATAC
TCTGTTGCAACTATTCAACTCTGCTGTTAGAATGTTGAAGCAGTCATAGACAATAGAGAAATGAAGATGTGTCATTG
TGATCCAATAAAACTTTATTTACAAAAATGGCAATGGGCTAGTTACGGCTTGAGGGCTGCAGTTTGCAGACTCTCAC
TTCAGAGCTAACAGTTGTTGTCAGGAGTCACTTGTTTTTGGAAACCTACAATGAGGTACTATAACACCAAAAAGAGT
TATCCCTTCCTTTTTCTCTCTCACTTTTTGAATTATGAGAAGAATTAGAAATGTAGTTAATGATAATGTCCAACCAG
TGTAATTATACTTGTTAGAAACACAGCTGGAAGCCTGTTGTCCAGTCTTATTTCTCCTCTGTGATCCTCATTTTCAG
AGGTTGAAGTCATAAGTTTGCCATGTCTACTTTCTGACAGGGGAATTATAATAATGTGGAGTCACCTTTTGTTTGTG
ACTTTGACAATGCTTCATTGACTTACTCACCAATTTTCTAATTTTTATGAAGACTTTTTGCCGAAATGTAGACTCAG
TCTTCTCTCTTGTCTACTCTTTCTATAACAATTAACAATGAACTTATTTACCTTTTTAACATCTTTTTAAAAATTTT
CTATACACCTTGAAAATGTGAATACAAAGTAATGCTGCATCATGTATATTGCCTTATTCACACATAGCCTCTTATGG
TATATCATATAAAAATGGAACAATACAGCAACAGGTTGAATGAACAGTAATCAGGTAACAGGAAAATGAGATGTCTT
TAATATTTCACTTAAAAACTCAATTTCCTAAAGCATACATATAAATATTTGGAAGTATAGTTAGAAGAAAAATATCT
TTAAAATATTTTAATTGATTAGTCTTATTTATAAGATAATTTTTAGGAGGCTGGTTGCGGTGGCTCACACCTGTAAT
CCCAGCACTTTGGGAGGCCGAGGTGGGCAGATCATGAGGTCAGGAAATCGAGACCATCCTGGCTAACACGGTGAAAC
TCCGTCTCTACTAAAAATACAAAAATTAGCCGTGCATGGCAGCGCATGCCTGTAATCCCAGCTACTCGGGAGGCTGA
GGCAGGAGAATCACTTGAACCTGGGAGGCGGAGGTTGCAGTGAGCCGAGATCGCGCCACTGCACTCCAGCCTGGTGA
CAGAGCTAGACTCCGTCTCAATAATAATCATAATCATAATAATAATTTTTAGGAAGCATCAGAAATATATAAGAAAA
AGATTATTTTCTTAATTGCTTTACTAAAAACACCTCTATGATTTTTCAGTAAAACTTGATTCTTATGTCATGTGTGA
GTGTGATCTGCCTCTCTTGGGATACTACTGTACTCATGAGGAGTGATTTTTTTCTCCAACGACCTCTTTGTCACGTC
AACAGGTCACAGGAATAGTGTACCCTAAAAAGCCACCTGCCACATGCTGCTGAAAATGTAAAAGTACACACATACAC
ACACACACACACACACACACACACACACACACACACCAAAATCAGGTATCACAAGCTGAAAATAAAATTGAGTCCAA
TTTTTTTTTAATTGAGCAGTTAATGTCCTTAAAACAAAATCCTATACTGCAACAAATACTTAGCCAGATCATTCTGA
TACCTCCAAACTGTGGTGTATTCCAAGATACCTCTATGATCTTTGATTTGATCCACAGCTTTTCAGTTATCATGCAA
ATACCTTCAAGTTTTATCTCATTTCTCAGTGCAAACTCATTAAAAATTTTCAGCTGAATTCAATTTTATAAACATGT
TGTGAATGTCCTCTTTATATAAGCAAGGTTGTAAGGAACTGGCCACATAAACAGAAAATTGAATAACATATGGTTTC
TGGCCTTAGTGATCTCATGTGTGAGTTAGGCATATGGGCAAAATCAGAACACTATAGAGTATAAGTCTAAAATGGTA
GTATTTTATAATAGAGGATGAAGAGGGTGCTGTGGGATCATAGGTGACAGATATAACTCCCGTTGTGGGACTTGAGA
AAGGCTTCACAGTCTGGAAACATTTAGTTGCTATTGAACACAAAATAAGACTCACTGTTGAGAGAAGGGAGAGGGAG
GGCATTTCAATCAAATTAAGATTCTGTGGCATATTCGGAAACTGATGTTTTTAAAAAGAGTAATGTTTATTACATTC
CTCTACATAAATTATATTTCTATGTAATATGAATGACAAATATTTAACACAAAATGCCTTATAACATTTGAATGAAA
TCCATCATATGACCTGTTATCTATTTCCATTTCCTTTTTGCTCATATCATTATGAACAATGACCTGATAAATTTTTT
ATAAGACTTTGCTGAATTAGTAAAGGATTATTAAGTTTAGAATGAACAAAGCTGACCAATCATTCAGGCAAATTTGA
CCGTTTTGTTGTCGCTTTTCTTATTTCTGAAACCATACAATTCCCTGAAATGAATAAGTACATATTTGATAACTTCC
TAAATTAAGGCTCAAAACACTGGTAATCTACTGGGCTTTCATTTGTTCCTTCTATTTGTCTAATCCTATCTATATTT
CTTTATATGAGCTATGAAAATATTAGATTTATTAAGTTGTCCTTTATCTTAATAGAGAAGAATGTTTTTCTATGACA
TTAAGAGGAATTTGATTTTTTTCTTTAATGATCTACTTTTAATTTTGGTAGAGTAGCATTGATAAGATCAATATTAC
ACATTGTTAAGTATGCATTACATGTTGATAAGATAAATATTACACTTAAAATATGTTTATCAAATGTATGAATGATA
AAAACGAATTCTGAAATGTATGGGAAAGATCTTGAATAAAGGTCTATGTACATTTCAAGGATGTCTACATATGCAAA
TTATCATAATATAATAACTATTGAATATGATTATCTTCACATACTTTCTTTATTTTTCATCTCTTAGATGAAATTGG
GTATTGTTTTCTTATAGCTGGAACAAAGCATTACAGAGAATTCTTAGTGTGATTTCATTGAAACTCACTGTTATATG
AGTTCAACAAAGTTTAAATTAGTCCATGACTTAATCATCCTTTATAAATCCTATCACTAGTATTCGGTAAGGACAAA
GTCAATTAAAAAATTAGCAACAGAAGCATTAAAAGAAGGATTAATAAATACAAAATAAGGGATGTGATATCTTTACG
TATTGCTGAGATGTTAGTGCTAAGGAAAAACTTCCCTGTTCATAATGTGAGGTGGGAAAAAGAAGAACTATTATTGT
ATATTTCTCCTCTCTAAAACTGCCTATCTGACTGTGTTTTTCTGTGTCAGCCGTATTAACAGATGTTTAATTTTACT
CACTTTAGTATATAAGGCATCATAATGTATGAACTATTTCAAAGGCCCTATGATGGCTAATTAAATAAAAATATATT
AAATATTAGCTGGACAAAATAAAATATGTATTAATTTTGGAAAAAGTAGATCAAGGTTTTGCAGATCTTTTCATATC
AATATATTCATTTGCTGAATAAGCTTTTATTGTTTACCAATATTACTAGTTTTATAGAGATGTAGATATCACCACAG
TATGACTAATTTTATAGGGACACAGATAGATAGATGTTATTTTATTCCAATCTTATTTTTACATATAACAGGTATAA
ATATGCGCTTGAAAGGAGTATATCACTTAGGAGTCAGTCAGAAAAGTAAAGATCTTCTAGTCTAATACAGTGGTTCT
CAGCCAGGGGTGATTCTGCTGCACGCTGAGGGATAAATTGGCAATTTCTGGAGACATTTTTGGTTGTGACAATTGCA
GGAGTGTTACTGGTATTCATTTGGTAGAGACAGAGATATTGGTAGACACTGTACAGGACACAGGAAAGTCTCTTACA
ACAAAGAATTATTCTGTCCAAAATGTCAGTTGTGGTGAGGTTGGGAAACACTGGTCTGGAAGAAGGAATTTACTATG
AGGAACTAGTTACGAAAGTATAGAGACATTTAACAAGCTGAACAAAGGATAGTGAGATGGCTCAGAGATTAGCAACT
GTGGCATGAAGCCACTACTACGTTTAGGTAAAAATAAGCTACCATTTATTCTTATAGTAATAATAATAATAATTATT
ATTATTATTATTTGAGATGGAGTTTCGCTCTGTTGCCCGGGTTGGAGTACAATGGTACAATCTCGACTCACTTCAAC
CTCTGCCTCCCAGATTCAAGCGATTCTCCTGCCTCAGCCTCCTGAATAGCTGGGATTACAGGTGTGCACCACCCCTC
CCAGTTAATTTTTTGTATTTTTGGTAGAAACGGGGTTTCACCATGTTGGTCAGGCTGGTCTCGAACTCCTGACCTCA
GATGATCCATCCACCTCAGCCTCCCAAAGTGCTGGGATTACAGGCATGAGCCACCACACCTGGCCCACTCTTTCTTT
TTTAATTATTGAGAAATATAAAAATATGTCAAAAGTAACAGGTGTGGTGGAGTTACAGCATGCACATAATGGGATAC
AGCCCATTATCTAATCTCAGATGGAAACTAGAAAAAAAAGAGAAGATCTTTGCTAAAGCACAGATTATGTGGAAAAT
CATTTAGAAAAATAGCTTATCACAACATTAAAATTAAATCCTTTAGCTGATCATTTTTCCTTGCTATTTTTTCTTTT
AAAATTGAGAAGACAGTGAGTTTTTTTTCTTTATTGTCATTATCTTGATGTCAAAAAATAATATGCACATTATAAGT
GGGAAAAAAGATAAGTCGAAATGAAATGAAACAATGCGAGGAAAAAAATGTCACAACACTCTTCAATTAGAAAAAAT
GACCCCCATCTTTCCTCCAAATAGAAATGACGTAACTGAAGTAGTGGAACTTTCTCTTCCATGGCAACTCTAGAGAA
GGGGTAGATGGCATGGGATTGTGGACAGATGGACACAGAAAGAGGCCTCATTTATTGTTATTGTTAAAACTTTTACT
TCTAGTAATAGTGACACCTCCTTCAGCATTTCTTTATCAATTGTCAATATTTTTTGGATCACCAGCATCACCTTCTA
TATGTATGTCTAGAAACCTCCTGTTATGAATTTACACTTCTCAGAGTCAAGACAGAAATGCTGTGAATTGGGCGATA
AATAAAATACCCCCCTTTTATTGCCTTGCTTTGTCTCTTAAAGAAAGATGCCTGTTGGGGGACTATGAGAATGCTTT
GTGCTTCTGGACCTCAAGGGACAAATCTATAATAAAAATTATGCATAGTGATGAGAAATATATATAATGCAAGTTTG
TAGAGATCAGTTAACTTATCTTGTCTAGGCAATTATTTCTAAACAATGATTTCAAATCATTAACTATAATATAGCCC
ATTCATACCCTCCATTTTTGTCAAATCCCTGTCACCTTCAAGGACTTGGCCATCCCATAGGCTGCTCTGCTTTTAAT
AGAGGAAGATGCTGTAACTCTTGGTACCATTGCCAGTTATGAATTTATCCATTAATGAACATTGCATTTAAGGCATA
GGTTTATCTCCTTCTCCAGGTATGAACCTGCAGGATTCCTACCTGAAGCTTAAGGGAGAATAAATCCACCTGGGACA
ATCAAGGACAGATCAACCAATCAGCTCAAAGCAGGTGTGAATTACACAGTTTATTTGAGTGACAAGGTAGCTAAAGC
AGGGATAATAAAAGAAGGGAGTGGGTTGATGTGGACAGACGAACTATGGCTTTAGGAAATTTGGTAGGGACTGAAAC
ATATTTTGTGTAATTTATGTGGGTCTAATAGCTTTTGAAACTTGTTTACAAGACCTGTGTAAGTGGTACTGGCATAT
TCATGCATGAGAAAACATCAAGGGAAAACTTAATAGTTCAAGGAGGTGACAAAGAAGAGAGGAACCAATTATTTTCA
CTAGCCGTCAAAAGCAAGAAAATAATCAGCTTGAGCCCTTCGGGGAAAAGATAGGTTAAATATTAAGTAACAGTTTG
TTATTATTCCAAGTGTTTTCTTAAAGTTGCTCCCATACTTTCCTGTTTTCTCTGAGGGAATTTAGTTTTTTTGTTGG
TTTTTTTTTTTTTTTTTTTATAACTGTCATTGGTCAGAGCTTGATTTGATGCCAGTCAAATTTTTTTAAAGAGATTA
TGAAAACTGCTTAAACTCTTCCAAAGGGAAGATGGGTCATTCTTAACATGTGTTTCAAGAGGAAGAGCATAAGAGCA
TTATATGGTAAGGCTGAAAGCAGATATCAGCGTTTAGGGGCCATGAAGAGGTAGAGCTCACATTGGTAGGATCATTG
ACTAGAATTCCAGAGATCAAAATTGTATGTTAGTCTAGCATTGGGGAGGACTTGTAGCTAGTATCTTCATTCTAGCT
TGGGAGCCTAGGAATCAGGTTAGGCATCTTGCACAGGAATGGGCCGATGGGCTAAAATCTCCTTGAGAGAGATGATT
AATCCAGGACAAACCAAGCAGTCATGCCAATGAATTACTTTAACAGGGTACTTCATATCCTCATCCTTTGGGCAGCA
CGGTCTTCAGAGATGGGGCAGGCCCCAGGCTGCAGTTGAGATTCTATAAACTAAGGTCAAAAAGATGCAGCAGTGAA
GAAGTCATGCTTATCTTGTATAAATCATGTTTTCTTTTCTTTTTAATGAAAATGTACATTTAACACATTTTAAAACT
AAATATTGACCCTAAAATTCCAACCAAAAAATGCTACATAAGTGGTATTTATTTTTGAATTTCCCTCATGCTCCTCC
CACTGTGGGGACAAGGAGTGGTGGTGGAAGAGAGATCTTTTAGCAAACCTGTGAGTAGAGAATTAGAAGGTAATGGG
AGGAAGGTAAAAGGAAAACATCATAGATGGATAGGCTCACAAACATTAAAGGCCTTCGTGCCTGTCCTTCATGCCTA
TTCATCCCTCTCCAGTATGTGAATCAATGTACTTGTTAAATATTCATTCACCTCACATATTTAGCATTAACCGTGTA
TCAGGGACGTTGTTAGACCGTTGGTTTACGATGATGTGTAAAATATCATTTGTAACTCAGACTAACTGGAAGTGCTC
AATATAATAAGATGTAATGTTATGGAACACTAAGTCTGTGCTGAAGACTTATCTCCTTTAATCCTAAAACAATCCTG
GTGGGTAGTCTCAATGATCATCTCCAAGTCACAGTTGAGGAAATTAAGGCTTCAAGAAGTTAAGAAACTGGACCAAC
ATCACAAAGGTAGCATCAGAGTGACAGTTTGATTTCAAAGTGTACTTGACTTCAAGGCCCACATTTCCTTGCACGTT
TAATATTGCCTTTCTCAGGTAAATATACCATTAAATGTGATACAACTCTAAGCATTTGAATTACTTACAACGTGCAG
AGTTAAAACCAGCATTATTTACACTATACTTCAGCTCGTTTATAAGTGAACTATTATTTTGTGGACTAACCTATGAA
ATGTAACCACATTGAATTCCTCTGTTAGGTACAGGTTTGGTGATTCCAGGGAATAGAGTATGACTGAATGCACAGGT
AGGGGTGAAGTGAACCCGGTCAGAAAATTTAGAGAGCATCGAGCAGATCATTAAGCAGCTGTCTTTCAAATGTGCAG
AACACAACTCATTTGTAATCTAGGGACTATCTGTATTGATTCTTCCCAGGGAAGTTACTTATTTTTATACATATGTG
GTGTGTTCTGTCCATAATACCATTCTACATGGTAATGCTCAACTTTATTATTTAAAAAAACTGCTAATAATGAGGTT
TTTCTTTGTATCACAGAAGCAGCAGGAGCAAGTTTTCTTTTTCCTTCCCAGTTTTTTTAAGTACTGCCAAGGAATGT
GATTTTGTCAGACTTGTATTTCCTATTAAGCCAATCTGCATGACTGTTCCTTCTACTAGCTTTACCTGTTCACTCAT
TTATTAATTCATCAAATATTTGTAGAGTGACTATTGTGTGCCACATACTAATATAGGCACAAGGATAACCAAAAACA
GACAAACGCTGTCCTTTCAAGGAGCTCATATAGTAATGGGAAGTTAGGAAAGGAGAAAATAAATATGTGGTATTTCA
AATGGAAGTATTAAAGTGTTAAGAAGAAAAGAGAAACTAACAAGATAGGGAAAAAGTGACAGGAACATGATGTTTTA
TTTTTTATTTATATATATTTTTTGAGACAGGGTCTCATTCTGTTGCCTAAGCTGGTGTGCAGTGACGTGATCATGGC
TCACTGCAGCCTTGACCTCCCTGGGCTCAGATGATCCTCCCACATCAGCCTCCCAAGTAGCCAGGTCTACAGGCATG
TACCACGATACCCAGCTAACACGTTTTCTTTTCTTATAGAGACAGAGTCTCACTGTGTTGCCCAGGCTGTTCTTGAA
CTCCGGGGCTCAAGCAGTCCACCCACATCTACCTCCTAAGGTGCTGGAATTACAGGCATGAACCACCATGCCCAGCC
GAAATTGATGTTTTATATATGGCAGTCTGGGCAGACCTCTTTGATGTGATATTTGAACAGAAATCTCAAGAGAGGGA
GTGTATTAGCCCGTTTTCATACCGCTAGAAAGAACTGCCCGAGATTGGGTAATTTATAAAGGAAAGAGGTTTAATTG
ACTCACAGTTCAATATGGCTGGGGAGGCCTCAGGAAACTTAAAATCATGGCAGAAAATGAAGGGGAAGCGAGGCACC
TTCTTCACAAGGTGGCAGGAAGGAGAAGTACTGAGGAAAGGGGGAAGAGACCCTTATAAAACCATCAGATTTTGGGA
GAATTCACTCACTATCATGAGAACAGCATGGGGGAAGCCAACCCCATGATTCAATTACCTCCACATAGCCTCTCCTT
TGACACCTGGGGATTATGGGGATTATAAGGATTACAATTCAAGATGAGATTTGGGTGGGGACACAAAGCCCAAACAT
ATCATTTTGCTCCTGGCCCCTCCCAAATCTCATGTCCCTTTCACATTTCAAAACCAATCATGCCTTGACAACAGTAC
TCCAAAGTATTAATTCATTTCAGCATTAACCCAAAAGTCCAAGTCCAAAGTCTCATCTGAGACAAGGCAAGTCTGTT
CTGCCTGTGAGCCTGTAAAATCAAAAGCAAGTTAGTTACTTCCTAGATAAAATGGAAGCACAGGCACTGGGTAAATA
TACCCATTACAAATGGGAGAAATTAGCCAAAATGAAGGGGCTACAGGCCCCAAGCCAGTCCAAAATCTATCAGGGCA
GTCAAATCTTACAGCTCTGAAGTTGTCTCCTTTGACTCCATTTCTCACATCCAGGTAACACTGATGCAAGAGGTGGG
TTCCCATGGTCTTGGTAAGCTCCACCCCTGTGGGTTTGCAGGGTAGAGCCCCTCTCCTGGCTGCTTTTACAGGCTGG
CATTGAGTGTCTGCAGCTTTTCCAGGCACGTGGTGCAAGCTGTTGATCGCTCTACCATTGTGGGGTCTGGTGGACAG
TGGCCCTCTTCTCATAGCTCCGCTAGGCAGTGCCCCAGTGGGGACTCTGTGTTGGGGCTCCAACCCCACATTTCCCT
TCCACACTGTCCTAGCCGAGGTTCTCCATGAGGTCTTCATTCCTGCAGCAGACTTCTGCCTGGACATCCAGGAGTTT
CCATACATCCTCTGAAATCTAGGCAGAGGTTCCCAAACTTCAATTCTTGAATTCTGTGTATCCACAGACTCAACACC
ACGTGGCAGTTGCCAAAGCTTGGGACTTGCTCCCTCTGAAGCAATGGTCCGAACTGTACCTTGGCCCCTTTTATCCA
TGGCTGGAGTGGCTGGGACACAAGGCACCAAGTCCTGATGCCGCACACAGTGGTGGGGTTGGGGGGGGGACCTGGTC
CACGAAACCATTTTTGCCTCCTAGACCTCTGGGTCTGTGATGGGAGGAGCCGCAATGAAGGTCTCTGACTTGCCCTG
GAGACATTTTCCCCATTGTCTTGCCTATTAACATTGGGCTCCTTGTTAAATATGCAAATTTCTACAGCCAGCCTCTC
CAGAAAATGGGTTTTTCTTTTCTACTGCATTGTCAGGTTGCAAATTTTTCAAACTTTTATGCTCTGTGACCTCTTGA
ATGCTTTGCTGCTTAGAAATTTCTTCTGTCAGATACCTTAAATCATCTCTCAAGTTCAAAGTTCCACAGATCTCTAG
GTCAGGGTCAAAATGATGCCAGTCTCTTTGTTAGTCATAGCAAGAATGACCTTTACTCCAGTTACCAATAAGTTCTT
CATCTCCATCTGAGACCACCTCTGCCTGGACTTCAGTGTTCGTATCACTATCAGCATTTTGGTCAAAACCATTCAAC
AAGTCTCTAGGAAGTTCCAAACTTTTCCACATTTTCCTGTCTTCTTCTGAGCCTCCTAACTGTTCCAACCCCTGCCT
ATTACCCAGTTCTAAAGTTGCTTCCACATTTTCAAGTATCTTTATAGCAGTACCTCACTACCTCAGTACCACTGGTC
TTAACTCCTGCGCTCAAGCGATCTGCTTGCCTCCACCCCTAAAGTGCTGAAATTACAGACATGGTCCATTGTGCCGA
GCCAAAATTGATATTTTATGTATGACACTCTGGGCAGACCTCTATGAGGTGACATTTGAACAGAAATCTCAAGGAAG
GGGAGAAATTATCCATTTACATATTTGGGGAAAGAGCATTCCAGGTAGAAGAAACAGAAAATCCGTAGTCTTGAGGA
ATGCCGTGTATATGCAGTATTTTTCAAACTTGTTATTTTGAAATACATATACACTTACAGGAAGTTGCAAAAGTATT
AAGAAAGATCATGAGTACCCTTCACTCATCTTCAGCTAATGGTTACATCTTACATAATTATATGTAATATCAAAGCC
AGGAAACCAGGAAATTGATGTTGATACAATCTATGCTTTATTCAGATCTCACATCTTACATAGCTATGCACAATATA
AAAACCAGGAAATTGATATTAACACAATCTATGCCTTATTCAGATCTCACCAGCTTTTACATGCACTTATCTGTGTC
TGTCATTCTATGCAATTTTATACCATGTTTAGAGTCATATAACAACTACCCCTATTTTGATACATGGTACTGAATAG
TTCCAGCGTCACAAAGGAACTATCTCAAGCCACCCTTTAATTGTCACACCCATCCAATCTCCCATTCTACTTCCTGA
ATCACTAGCAACCCCTAATCTGTTCTCCATCTCTATGATTTTGTCTTTTCAAGGGAGTTTTCTAAGTAAACTCATTT
GGGGAAAGAAAGGAGATGAATTGTTCTAGCCACGGAGTGGAGAACAGAGAGTAAGAGTACCTATTGAAGCAGAGGGA
GTCATTGCAATAATTCAAATGAGAAATAATGGTGATTCTAAACCAGGAAGCTTTCAGTGAAAACAATGAGAGGTACA
TGGATTCTGGGTATTTTTGGAAGGTAGCACTACCAGGTTTGCTGATGAATGGGGTATGGGGTGGGAAAGAAAGAGAA
GAGCCCAGGATGAGTCCAAGGTGGATAAGGTGAATAGAATTGAGAAAATGGTAGAAGGATCAAGTTAGATGGTAGAG
GGGTAAAGGTGGAAGCAATAATTTTGTTTTGGAATTGTTAGGTTTGAAATCTTGTTAGACATCCCAGTAAAGTCACA
AAGAGTGCAGTTGGATGAAAGTATGGGATTCAGGGAAGAAGTATGTGCTAGAGATGCAGATTTGAGAGTCATCTGTG
TGGAGGTATTATTCAAATTCAAGTCCCCTTGGAATGAATGGCTATTCAGGCAGGGTCTTCATAAAAATGCTTGTTGC
ATGCCTGTAATCCCAGCACTTTGGGAGTCTGAGGTGGGTGGAACACTTGAGGTCAGGAGTTTGAGACCAGCCTGATC
AACTTGGTGAACCCCCATCTCTACTAAAAATACAAAAAAAAAAAAAGTTAGCTGGGCGTTGTGGCACATGCCTGTAA
TCCCAGGTACTTGGGAGGCTGAGGCAGGAGAATTGAGCCAAGATTGTGCCATTGCATTCCAGCCTGGGCAACAAGAG
CAAAACTCCGCCTCAAAAAAAAAAAAAAAAAAAAAAAAAAGCTTGTTGCTTCAAATTCATGTCAGTCTGTAAAATTA
TCTGGGAAGGCAGTACAAAAACTGTCACTTTGACTACGATGTTTCTGGTGACCCATCTTCATTGATCAGTATGGAAA
AGGCATGTCTCTGAAAATCTCTGAGAGTCTTTGATACAGCAAGAACATAAGGATAAATCATTCTTCTATGTTCATGG
TTGTAGAGGATCTTGAATGTTTAATGGCAGAATAGCCAGATCACACTCTGGCACTTCTGTATGAGAGGCTGAGGGAT
GTTACTGATTCACCCCGAGAAATATTTACTACTAAGGGGACAGAGGCAAAGGGGATACAAGACTTCACCCTGAGCTG
TAGCGCTCCCTCCTTCCCTATCCTGCTTTCATTCTTCACATTGTTTTCCTTCTTTCTTTTTTATTATTATACTTTAA
GTTCTGGGATACACGTGCAGAATGTACAGGTTTGTTACATAGGTATACATTTGCCACGGTGGTTTGCTGCACCCATC
AACCCGTCATCTAGGTTTTAAGCCCCACATGCATTAGGTATTTGTCCTAATGCTCTCCCTCACCTTTTCCCTGTGTC
CACATTGTTTTCTTTCTTTTTGAAGCCTCTCATTCACTAGGTTTCAATCCTGCCTTGCTAGTGTTCTAACTCTAAGG
CCTAGGCAAGTTATTTCACCGAACTTAGCCTCAGTGTCCTCATCTGCAAAATGGATAGTTTTATGATATCTTCAGCC
CTTAAAGTCAATGGTTCTGACAGCTAGGGTGTACTATCTTCTTGGATATCAGTCATCTCAAGCAAGCCCTCCTTTTT
TGGACCTTCTTTTCACACACTTCACATACCTTAGAGAACATAATACACATCCTCTTTACTCAGGGCTTATTCTTTAT
AACAGGCTTCCTAATTCAATTAACTCAACTTTTCAAAAATATTAGTGACTACTGTGATGTAAATAAATTTGCATTTT
ATAGGGGTCTTAGTAACCCAGAAGGGAGTGGGGAAAATTAATATATATTGAGAGTTTATTAAGTGCTAGGTACTGTA
AATATTTTCTTGTATTTAATCCTCCGAGTAATTCTACAACAAAGATATTATCATTGCTATTATGTAAATAAAAGAAC
AAAGTAGAAAGAAACCCACGGTCTTGTATAAGCTCCCCTAGTTGGTGGGTATTGAAGGGAGTATTTCAATCTTTGGT
AGCTTCTGAGTTTTTGTTCTCTCAGGGAATCTGCCAGATGTCCAGGGCACCTGCCAAACCCTATGAGGCTATAAGAA
AACCATTAAGGGTCTTAGATTACCCAGCTTTTTGGGAGTTAGAATTCTGAATGAAATTTAGTGTTCCTGCAGCTACA
AAGGAATTGAGTTAGGGAAGTGATGACTTTATCTTTAGCTACATTGGTTATTTTCCTTATAATAATCCTGGCTTGGT
AGATTAGAGGCAGCCCGAGTAACCCAGAATCGCTAAAATAGAAGTGCGAGCTCATTGCCCGCTGTCCTTCACTATGT
TTGCATATAGGAAGCAAGAATAAAACAAGCATAAAATAGGCTAACTAGCTTGTCAGAGCTCTTCACACCAAGTCTTT
GTGAGTTCCAATAAGACACTGACTATTATTAAAAAGACAGAGACTCCACATAAGTAGGAATTTATTGTTTTCCTTTT
CAGTCACCAAAGGACAATCCTCTGCATAGGTTAGCAAAAAATGGTACTGATCCTATAATCTCTAATATTAAAGTTTA
GATTTGGCAAGCTGTACATCTTATGTTGTTCATTAACAAAAAACAATATTGATTGGTATCTTGTACTATAACTTGTA
CTGTGGGTCAAATTCCAATACAGCAAATACCATTGCAATAACAATTCTACAAAACTACATCAAAAAAACCTTTCATG
TTTGAGCCAACAGCCTGATAGTGCTAAGGACTTTGAGTACAGTATGCTAGAAGATTCTTAACAGTTATTTGTCCTGG
ACAACAAAGGTTGACTCCATTAAAAACATAGCCATCAGTGTGGGATTATTTCCAAATCAAGCTTTTGGAAAAGTCAA
ATGAAAGTTTGCAAGCAGGTGGGGCATGGTGGTTCATGCCTGTAATCTCAGCACTTTGGGATGCTGAGGCAGGCGGA
TCACCTGAGGTCAGGAGTTCGAGACCAGCCTGGCCAACGTGGTAAAACCCCCATCTCTACTAAAAATACAAAAATTA
GCTGGCTTTTGTGGTGCATGCTTGTAATCCCAGCTACTCAGGAGCCTGAGGCACGAGAATCACTTGAACTCGGGAGG
CAGAGGTTGCAGTGAGCCGGGATCATGCCACTGCACTCCAGCCCACATGACAGAGTGAGACCCTGTCTTCAAAAAAG
CAAAAAACAAACACGCAAACAAAAAAAAAAAAAACCAAAGTTGGAATGCAATAAATGTTCATTGAATGAATACTGAA
TAGGGAGTTTCAGCTAATCCACTCAAAATAGTGCTGAATTTCCAGCTCTAAGGTCAATGCTTGGCATATATATCCTG
AAGGAATGAATGGACACAGAGTAATTTTTTTTCTAAAATGCAAATTCAATTATGTCACTTCCCTTCTTAAAATCCTT
CAGTAGCTTCCCGTAGCCTCCAGCATATTATTTTGAATAGTGCTTCTCAAACTTTGATGTGCATCAGAATCACCTGG
GGATTTTCTTAATTAACTGATGCTGATTCAGTAGGTCTGGGGTATTGTCTGAGATTCTGCATTTCTAGCAAGTGCTC
AGGGTTATAGCAATGATTTTGGCCTGCAGACCATACTTTGGGTAGCAAAGACATAAGCCACTTAACTTGACATAAAA
GACTGTTTAGACCCTTAGTTTCTCTCTCGCTCTTTCCCCATTTTGAGCTTTTGCTCCGGTTCATGTTTTTCCCTGAA
AATACCGTGATCTTACATTGTCTGTCTGGATGCTGAATTTTCCCTAATTCTGGGCCTCCATGTAGTTTTAGGTTTGA
CATCACAACCACCAAAAGATTTCCCCTTCTCCCTTAATCTTGGTTAATGTCACTCTCATGTATTATACTGTTAATGA
AGCATTGAGGACATAAAACTTATCAAATATTTTATCACAATCAATGATGGCACCAGTGATAACATCCAAATGCCTGG
GTGAGTAAATAAGAGGAGAATAGGGGACTTGTTGTTAAACTAAGTTTGCAGAGAAAAAATGTACTGATTATAATTAA
ATTGGATGTTTATTTGTTATGACAAAAAAGGAGCTAGAGTCTTTTAATCCACCCCTTGGCACCACTGCTTATCTCCT
TGTAACATACGTTTGATTCCCATGTCTATTTCTTCCATATGGGAAATTTCAGCTCCCTAAACATCACCAATACAACC
TGTTGATAAGACAAAGTTAAATTTATTGCTTACTATGGTAAGAAAGACCACAGCCTGGACAAAGCTTTGGTAGTATT
TCATAAGGAGAAAGGTGAGGTTGGATTTCATTGGGAGTATGAAGCTTGGTTTAAGATTGGTCTTTCACTGTGGGGGC
ACAATTAGGATTGGGTAAGGATCATGGTATTACAACTTAGTTTGGTGGAAACAGCACAGTGAAGATTTCTAGCCAAG
AGGCTCAGAGACTATTAAGGTGTGAACTCTATTGATGTTTTTTGTTGAAGAGTTGATGGGAGTTTGGGGAAGTTACT
TTAGTGAACAGTCAAATTATTTGCCTGGCCAAGAGTTATCTGTAATAGGAAAGTTATGCTAATGAAGACAATGGAAA
GGTAAACCATGTTAATGTCGACAGCCAGCTATGTGAGCATAAGGGGTAGGTAGCTTTGGTCCTCCATGTCCAAACTG
TTTGTAGTGGTAAGTGATCTTCATTCTCACATAGATTGAAAGCTTCCTGAGGACAGGGCAATGTCTTTGTAAACTTT
AAAATATCTATGTCCTGCACATCACCTGCCGTAGACAAGCATCTAGTAATTGACGGTTGGGTAGATACTGAGGGAAA
ACATGCACCAAATAAAAATGGCAATAGGACACAAATTCACTATCATTTGGAAGAATAACAGTGTTTTCCACTGATAT
TTGCTACACACAGTGGGGTCCACAGAGCAGCAGTACCACTTGGGAGCTTATTGGAAATGGAGACTCTCAGGCACCAC
CGCAGGTCCAATGAATTAAACTCTGCTTTTTTTAAGGTCATTTGTATTCAATTATTATTTTTTTCTTTTTTCTTTAC
TTTCGATGCATTTTTCTTTATTTGTTTTTGAGATGGGGTCTTGCTATTTTGCCGAGTCTGGTCACAAACTCCTGAGC
TCAAATGATCCTCCCACCTCAGCCTCCTAAGTAGCTGGGATCACAGATGTGAGCCACCACACCTGGCTTGTATCACA
TTAAATTTTGAGGAGCAGTGCTTTAATATCTATTCCATTCTCATCACTTGATGAGGTATTATTAATTCCACTTATGG
ATGTGGAAGTTGAAGCCAGAAAGTTTAAATGACTTGTACAAGGTCAAACAGCTTACAGGTAGTTGAGCCAAGAGGCT
CTCAAGTCTTCTGCCTCCACAAACCCCTGTTCAGCTGCTGCCCTACAATGGAATAAAATATACTAATCCCAGAGGGA
CAAATATGCTAAAAATCTCAATATTATACACTTTGGAAGGTGCAGGTGCATTATCTTTCAATTCTAATTTCTCTTTC
AAGTTTTCTGATGCATAAAAATATGAACAGCAGGTCTGAGCAATGTTTAGATGCCGTGCTTTGATCCTTTTGCCATT
CAAGATGTTTGATTTGCATTCTGCCAAGGAATGTCTGGTAACCTCCATGATGCAGACCACACCATTAGTCAAGAGAG
AGCTGACGTACCTTCATCTGAGAGCTGGCTGGCTGTGAGCTGCTCAGAGGGAAAGGATTTCTATTTACAAATTGTAT
CGATTATTTATAAATAAAAGTTCCCCTTGCTTTCTTCAGTTGTAAAATCTGCAGTTAGAGAGTCGGGAAGAAGATCA
AAACTGCATACATTTGCATCTGCCAAGCCTGATAACTAGTTCCAGAATTACAGAAATGGTGCTGAAATAGCACCTCA
AGTACCAGGCTCTATCAAATTTAATCTATCCATAAGGCAACTGCCAATTATATTTTAGAGAAAAAATGTAGACTGAA
AAGATAGACAATCCAAGTAGCAACTCCTGTAAAATTATATGCCCATAGGAGCAATCTTGAAGATATAAATATTGGTA
TGTTTCTCCTTCATTTATCATTTATCTGATCATTTGACAAGTATTTATTGAATGCCTGTTAAGGGTGTAGATATATG
TGGTGAGGCTGCAGGTGTAAGTAGGTCTTTCTGAGGATATGCATGAAGTTGATGTTCATAACTTGGAGATGTGTGTA
TACAGACTGAGGATTCCTTCAGTGGATATTAAGAAGTGGAGTAATAGGCAGTAAAGAATACACTAGTCAGTTGTGGT
ACATAAACACGTCAGCACCACTTAGGTATTAACTTCCTGTTTTGTTTTGTGTGTGCTTAATTACGCTGTTTATTAAA
CAAGCACATCATAATCTGCAGATATTGTCATAAACAGCACAATAAAGCCTGCCACATCAGAATGTCATCTATCAAAT
TAGGTGTGTTCCTCAGCTGTCCCGATAGGCACACACCTGTGCCTGTAAATAGGCGCTTGGCGGAGATTGCTTCCAGG
TGTGGATCTGTTGGGCGACCTTGGGATGTAGGGCACTTTGGAACCTTTTCCTCTAGCTTCAGGAATTAACCTCTGGG
CTTGGTTCCATGCCAGCTTGCATTTTGCTTTGGGACAGTAACATGTAAAGAATATGCCTGTGAATTTAGGGTTACTG
AGAAGTCCTCATAGAAGAAGTAAAATTTCCTTGAGGAATGGGAGTCTTTTATTCAATCCAGGTTTAATGCAAGGCTT
GGTGAACAGCTCCAGAAGGTTAATAATTGCGTGCGTGTGTGTGTGTGTGTGTGTGTGTGTGTGTGTGTGTATCCTTT
TGTCATTCAAAAGTATACGTATACACACACACCTGTACAGCTGATGATAAATATACATTGTATCAATGAGTTCAAAT
GAAGTGTGCTATTCATTCACTGAGGAATGGGCTATTATAATGAACTATTATGATATTAGAAATTGTCAGGGCAATAA
GCAAATAATACATACGGTTTTCAACAAACTTTCTAAGTATTGTTATCAGTGGGTTTGCTTAAATCTTTTTTTACAAA
TTTATTTATTTTTTTGAGACGAAGTCTCGCTCTGTCGCCAGGCTGGAGTGCAGTGGTGCAATCTCGGCTCACTGCAA
CCACTGCCTCCCGGGTTCAAAAGATTCTCCTACCTCAGCCTCCCGAGTAGCTGAGATTACAGGTGTGCGTCACCATG
CCCATCTAATTTTTGTATTTTTAGTAGAGACGGGTTTTCACCATGTTGGCCAGGACAGTCTCGATCTCTTGACCTTG
TGATCCATCTGCCTCAGCCTCCCAAAGTGCTGGGTTTACAGGCGTGAGCCACCGTGCCCAGGCAATAGCCCCATTGC
TCAGTGAATGAATAGCACACTTTATTTTAACTCATTGATATAATGTATATTTATCATCAGCTATACAGGTGTGTGTG
TGTGTGTGTGTGTGTGTGTGTGTGTGTGTGTGTGTTGAATGACAAAAGGATACACACACACACTCTTATTAACCCTC
TGGAGCTGTTCAGCAAACCTTGCATTTTTTACTTTCATTACAGTGTGTAAATAATTTAGCAAATTCTAATTTGAACC
TGATATCAATTGAGCATTTAATATTTAGCCAAATATTTATCAAGTGCTGACTGTGTTCTAGATGCTGGGGCTGCAAT
TTCGAAACAGACCATTGAGGCCCTCATGGAGCTCACAATAAATGATCTTCCTTAAAGTATCAGGTCTCTGGTTTGTT
ACCGTATTTTTTAAATTGTTAAGGAAAGAAAAAGGCCCTATCTTTTTGTAGACAAACATGCCCTAAGTGCTTCCAGA
AATAATCTCCATCAGGTAATGCAGACTGTGTGTGGAGTGAAATTGAGTCCAATCCATGATCCAGCAGAGTTTCAGCC
CAGGATTTCTTTAGAGCCTTTGCTACACACAAAGTTGGCTGATGTGCCATTCAGCATCCCAGCAGCTCTTTCTCTTC
ACACTAGCAATGGCAAAGCTTTGTGCGGAGGCATTGCTGGCTGCTCTGAACTAAAAGCATCCGTGGGGACCGAAAGA
GGTTTTTGCACACCTTATTAAGGTAGGCAAGTGTGTCTGAGTGTGTGTGTGCCTAAAAGCTGGAAGACATCTGTTGA
GAGGAAAGTGCTCTTCTGTGGGTCTGGCAGCTTTTCTGTAAGTCTTCTATTCTGATGCAGGAGCGTGTGAGCAGTGG
GTGGGAGGAGATGCTTTGGTACTTGGAATGCTGAGGTCCGGATTAAGTGGTATTGTAATAGCTAGTTAGAGGCAGAA
TAAAAAGCTGGGAATCAAAGCATTTAAAAATGCATCCTTCCATTATTTGCTCTCAAGTTAAACCATATTCATTCTAG
GGGAAATTAAAAAAAAAAAAAAAACACAGCAAGGGCAAGTAGCCCAAATCTGTAAGGTCTTTGAGCTTCTCTGTTCG
TCCAGCTTTTGAAGTCTTCCTACAGCCAATTTGTTTGGCTCCTCTGGAGGGGGCAATTCATATCCACTTCCCTCTCC
TGGAGCATTTCTTTCTTCTATACTCCATCAGGGAACAATAGAGTTTAACAGTAACAGGCAATTTTTTTTTTTTTTCA
AAGCTTGTGCCCTCTTCTGCGTTTAAAGGTGTTTTTTAAGAGACTCCTGCTAGGGGAATCTTGGCGCCTGTGTGTTA
AGACGGCAATTAACTTTTAGTATCAGTGCTTACATTAAATTTTCTCTCTTTCTGCTTTACTAAAGCAGTCATTAAAA
TTCAGTGTGAGTACCATGAAACTTTATCATAAAACCCTGCTTTGCTTAGAGAACCTTGATTGTTTTCTGAAAGCAGC
CTTCTCAGTTTATATATACATAGCTGCCTTCCTTGGAATATCAAATTGCTTTGTGTCACATTAAGAAACACTAGGTT
GAACCTCTATACTGTGTTTTATCTGAGAAAAATACTACTGCAAAAAGTTTGATTTGTTCAAGTTTTAGGATGAAAAT
TTCTTTGTAACAAGTTATTTGAGTTGCATACTATGTCATCGTATATCTCTTTAGTTCAAGTAATTTTGCAATTAACA
TACGGTTATGTAAAGAAGATAATGATTTATTTTTTATTTATATTTTTAAAAGTTATTAAGTGAGGTTTTCCTTTCAG
TAAGAGTTTAGAAAAAATAGCCAGAACAAGTAACTGGACTTGGAAGATAAAGATACCTTTGCACTTCTAAATTTTAC
CTTTGTACACTTCGGTTGTGATTTAATCATTGAAATGCCTCTGCTTTGAAGTAAATGCATCACTTATGGTGTATGCT
GTGTTTTAATAAAGGGAAAACAGTTATGGGTTCTCTGTTGCACATTTGAATGTTGTTATTTTTTGCTGTATTTAATA
ACCTCTTTTTTCTCTTGTGAGGTTTACTTTGGAAATGAGGCATGTTCAAAAATAGGCTGACATTCAGCTTCTATGTT
TTAAATTTAAATGCTGTCTGTGTTTTATCACATCTGGAATGTGTGGGGAGAAAAGATACCAAGTTTTATTATTTAGA
TTTAATTGTAGAATTGCAGATTGATATTTTTCAATGCATTTTCATTATAGTTTCTGCCATGGAGGCAGCGTGAGGGC
TTTCAGGAAGATGGAGTGGTGTAATTACCAGGTGCGCACGTTCATTAATCCTTCCTGGCTAGAGAAAGCTTCAAGTT
CTTCTCCAGTGGCCCATTCGTAAAGCTATAAATATCTAAATTGTGTCAGCCAAGAAGTCACACAGAATGGTGGCTCT
TTTTGAGTTCAATTTCATGCACTGTTGCTTTGGTCTTGTGAGGAAAGCTCTGAATTCCTTAGGATAGTCTTGGTTGT
GAAGTTCCAAAAACAAAATATCAAATCATTAAGGATTTAATTTAAAATACATACTCTTCTTTCACAAACTAGATGAT
TGCAGTAATGTGGATTATAAATTTTTTTTTTTGCTTTATTTCTTTAGAGCTCCTCTTTTTATTTTGTATGATCAAGA
TTATAGCTGAGATTTTGGTGATTTTTTTAAAAAGATTTATGGCTTATGGTCCATCAGTCTCTCCACTACTTCAAACC
TGTGTACCCCTGTATATTATCTGCAGTACTGGAATGTTTGCATTGTATGTGGAAGCTATATACGATTTGGTAAAAAA
TAACACTTAAAGGTCTTCGCTAAGAGTGCTTATTTAATCATTAAATATCCCTTAATAAAAATAATTCCAGAGATATT
GTCTGTGTACAAACTTAAAAAAAGAGAAATATAAAATACTGTGATGTGAATAAAATGTATAGCAATACACTCCAATA
ATACCATTCTTATGTTTTCCCTTGTTCTCAACTGAAATAACTAAGCTAATAGAGACGTCAGTAAGGAATGTGTTGTT
TCTTCATAATACAACTACAAACTCATCTGATAAGAACAACCTGAGAGTGAACGTTAACTTTCCTCATTAGAAAGATT
CAATTTAACACATATATACAAATACATTTTTAAGATAATGATATTTGCAGAGTTTTTGTATTCTATGGAGTAAAGGA
GAATTATCACATATTCAAAGTAAAGGTATAAAATACATCTTAATGTTTTACTTAAATTTTAAAGGGTCCAAAATATA
CTAAAATTGTTTTTCTAATTCTTTCCTATGTTTAAACGTGCCAGAGTCATTGGAAATAGGACATTCTTTTTCTTAAG
AAGATTTTGCCCAAAATATTTAAAACTATTTTCTTTTCCCTTGATTTTACAATTTCAATATTCATGGATTTTTCTAC
TTTAAAAATAACAGTAGTTTTTATGATCTTAAAACAAATGTTTAAGGGCACTTTCGCTCTCTGGAGACTATACCATC
CACATATTTATTATCAGCAAAAGAAAGGGCAGGGCATACTTTTATTTGAAGTTGAGTATAAAAATGTGTCTGTGTGT
GAGTGTTATTAAAAAGATAAGTGAAGAGACAAATATAGAATCCAGGAACATTTTCAGCCTGGCTTTTACTCTCTCTA
AAAATCTAATGAAACCCTTGAGCATCTCTTATCTCAAGGTACATTAGGAACTGTCCAACACTATGATCCGATGGGAG
ATCAGTATATTCATATAAAGAAGAAAATTTGTTGTTAGTGAAAGTCAAGTCTTTTAAAAAAATAATAGTTACAGCAT
TTGCAATATACAAGCATAATAGATTTACTCAACGCCCACCCCCCATCTTTAAAAAATCAATTTCCGACAGTTGTCTA
CTTTAAAATTGAACATATTTGCTACCTGGAGGGAACATTGTAATGTAGCCCATATGTGGTATGCATCCTGAAGAAAA
CCTGAAATTATAGAGGAAGTTATCCTGCCTTCTTTCTTCTGTTGAATGAGTTAAAATATATTAACAATTTGCCTTTC
ACTTTGTATTTATCATTTTGTATCTTTGCATATTTACATATACATTCATGTGTACAAGGGCATATATACTCACAGGT
CAGGGCTATTTAAACAGCTATTTATTTGAATATGCCAGGGAAAATCTCCAAGATATAAAGAAGCAGTTATTAGATAC
TATGTCAGTATAGAATTAACAGCCATCTTTTTTAAGATGGAAGAGAAAATTAATTAATTACATACAATTTCTAACCT
CAAGACATTTTCTTTCTGGAGACAAGGAATACTGAGGTGCTCACGATAGTGAAGACTCAACAAGACCCTAATAAAAT
AGATGAGGATAAGTAAAACTACAATAGCCAATAAAAAACAAAAAACAATAAACCATGTTTCGCTGGCATGTTGGTGA
GTATCTCTGTAATATCTGTCAATAAGGGTCTCTGTAGATTTGGAGTAATGTTCAGGAACTACCTGTACTAGAGAAGA
CAGTGGAGAGGACTCCAGTGGCTAAATTCTGCTGCCTTTGCTTCCAGAAATGTAAATAATAAGGAGGTATTGTGGCA
TTTCCTGGAAGCAGTAGTCTTGTTTCATGGTCTGACTGTATAAGAATGCCTAGAGAAACATAACCTCAGCTGACTAA
ACTCCCTTGATGATTGTCACTTTGTCACTGAACTCTGACCATACCTTTTGCCTCCAGAGGCAAAAGACGGGTGAGGA
AGTGATCTCCTCATCTGGTTTTTAAACAAGTATATAACTAGAGAACTGGATTATCTCCTAAACCCACTCTTGTCCCT
GGAAAAAGGGGAGTCATCCTATCCGTTTCTTAGCCAATTTATGTATACTCTTAGTTTGAGAGCATGAGAAGGAAAAC
TATTTTCTTTTCTTACCTTGGCTGGGTTTTTAAGAATTTATTTTTAGTTTAATCAAAATAATATTTTAAAAGGTAGT
AAGCCTCTCATAAGCAGTTTGATCTGTTCTAAAATAACTTCAATTTTTCTTTTTTTAAACTTTCTTTTATCTTACAC
ACAAAGTATAATAGTAATATGTACTCACTAGAACAAATGAAACAGGATGGAGTCACATAGAGAAATATATCATATTC
TCCCTATCCCCTCCCTTAATATTAACATTTAGGTGTCATGTGCTTCTCCATTAATTTTCATTGCAAAGGCCTAAATT
TTCTTCCAAGAGTGAGGAGTAGCAGCACGGTAGTTTGGACCTGATATAGCTCTCTTTCCCTAGCCTTTTGCTTAAGT
GCTTTCCTAGGGGCTGACTTTACTTACCTAAAGATGTTTCAAGCAAGGGCTCACATTTTTGGTAGCAGAAGACACTT
ACTGATTGCTCTCACTAATAATTTTGAAAGGAATGTCAAAATCTGGGAGGATCATGAAAGAAATATCAGAAATTTCC
TTTCAGCTGCCATTCTCCTTAATACTGTTATCAATAAATTCAGCATCTCATATGTGATAGCAAAAAAGGTGCTGCCT
TTTGTTCTTGCATCCTGAGGTTCTTACCTAATACCATGGTAGCAATAAAGATGGTGAGAAAATTGCTTCTTCTATGG
TGTTCAGGTCCTGAACGAGCACCCTCACCTCCACAGACGGTGGCAGGTATTCAAGCATTTTACAGACTTTGGAGTTA
AATATAGCAGTGTTATTCTAATTTAGGTATGCCACCACCAGCGGCACCGGCAACTGCAATAGGAAAAATGATTGGCA
ATGCCAGCTATCTGATGTTTTCATGTGCCAGGTGCTGTCAGTTCTTCACAGTATTACATTCCATCCTCACAACAAGA
GAGTGCCAGTGAGTGTTGCTGTGTGCCAGTGCCCAGGCTAAGGGCTTTGAACACATTACCCTGTTTTATCCTCATAA
CTTTCCACGTTATTTTTATTCCTGAATGAAGAAACAAGTTCTCTGTAGAGATGCTGTCATTGATCCACTCATATCCT
TTCACATCCGTTTAACATTTTCCCTGCTGTGCTTTTACTCCCAACAACTAGCTCCCTAATCGCTCTGTTGGAGGGTG
GCCTTGAGGCTGCCAGAGCCTATTTGGTCTGTGTAAAGAGAGAGATGGATCTATCCTGGAATTTATGTCCCTGTGTG
TGGGAAGCCCTTAATCAATGACTGCTGGTTGCAGACACATAAATACGTGAGCTTTCTTGTTCCCAACTGAGAAATTC
AGAAGTGTGAATGGCACTGCCACCCTGGGCTTTTATGCCATATATGTGTTTGGTCTGTTTCCCTTCCCAATCTCACT
TCATTTTCCCTTACCAGTGTTTCTTGAAAACACATCCCATTAGATCATTTTTGCATGAAGCTTCATCTCAGAACCTC
CATTTAGGGAACCCAAACTAAGATATTCTCTAAAATAGAAACTTTATTGATAAAGTTTCCAAACTGTCTTAGTAGAT
GGCCAATATAAGACCAAGCCAAATCTTTCTGGGTCCAAATTCCCTGTCTTTAATTAATAGACTCCATTACAACACAT
TCTTCAATCTTTAGTCAGCAAACACTTACCACGTGCCTATTTTATGGCATATTATATTTATACCATAGTTAGGATAT
TATGGTTCATGAATATTTTATATCTGTACACCTGAAATTCTATTGACCTCTCTGGGCCACAGTTTTGCATCTGTAAA
ATCAGCACAATAATGCTACTTATCTCATAGAGTAGACTTAAAAACGAATGAAATGATATATGCCAAGTGTTGAGAAT
CACAATTGGCAATTACTCATGCTCATTAAATATTAGCTGTTTTTATGAGTATTGTTTCATTTTCGGTGCATAATATC
CTATGCAAAGAACAAAAGGTATTGGTATAGGCATTGAAACTTGAAGCATAGAAGAAAAAGTTAATTAACCGGTGCCC
CACTAGATGCCTCTAACTGCTGGCTCCGTGTATCCCTTTAGCCTTGGCTCGTCACGAGAAAACCTTGGAGACATTTC
TGCTGGACTCAGCAGATCAATTTAAGAAAGATGAATGACATTTTTCTTGAAATGTATTCAGTCATAGCTGCCTTTTT
CTACTTTCATATTTTGGAGTTCTTAGAAAAAATTAAGGACTCCTTTTTTTAAAGAAAATGGTATAAAAGAAAATGCA
TATCACTTTGTCACTTTATTATTGTAACCTCATCAAAGTATTCAGTGTAAAGACAGTAGCCAAGTGAACTCTTCTTG
TAATGCTCGGAAACCATTTTAGCAATGGTAAAATTGCTGCAATTTATATTCGTCAAATTGCATGATTTGACTTATTT
TAGAAAAGTTATTAACTTCTGAAGAGAATGCTTCAGAAGCATTTAAATGAGTACAAGTTATCACCAGTGATATACAT
AAATTTCATTTCAAAATATACTTCTAGAAACTGTACTTAGTTAGCTATAGTATTTGTACAAGGATTAATTCCTATTT
CATTTTGTAGGAATTTATTTATGAATGTCTATGGCCTGCCAGTGTAAAGCAGACTTAGAGCATCATCTTTTACAATA
ATCTTTTTTTTTTTAATCAAAGGGGAGATATTCTGGTAAAACAAAACAAAACAAAAACAATAGTTTATTCTGCATTT
TTATTAAGTCCCTCTGTAAGTCATCCCTGAAATGGGATATGTAGAGTCTTATATTTATTTATTTCTCAGAAGCTTAT
TGGAGGTGATATGAAGGATTTTAAGACCCTACTAACTAACAAAACAACAATTTAAAATTAATTTTCAAAATACCTTA
ACAAATCTTATTCTCCTTATTTTCAAATTCTTTAACAATGTTTTTCTTATTACTAACATAATATCTTCTGATGTAGT
CATAATAATATCTAAAATGACAGGTCTAAGTAACTTACATGGATTAATTGAGTCTTCTAAATAGTAAGGTAGATGGC
ACTATTACTTCTATATGAGAAATGAGGAAGTAGAGGTATAAATAAGAAATTTTTTGGCCGGGTGCGGTGGCTCACGC
CTGTAATCCCAGCACTTTGGGAGGCCGAGGCGGGCTGATCACGAGGTCAGGAGATCGAGACCATCCTGGCGAACACG
GTGAAACCCCGTCTCTACTAAAAATATAAAAAATTAGCCTGGCGTGGTAGTGGGTGCCTGTAGTCCCAGCTACTCGG
GAGGCTGAGGCAGGAGAATGGCGTGAACCCGGGAGGTGGAGGTTGCAGTGAGCCGAGATCGCGCCACTGCACTCCAG
CCTGGGTGACAGAGCGAGACTCCATCTCAAAAAAAAAAAAAAAAAAGAAGAAATTTTTTTGAGTGTATACAGTTAGA
AAATGGCAAAATGGGAATTCAGACCCAAACAGTAAGACTCAAGGATACCTTTCTTATCAGTATGCTAATATGAAAAC
CTAAGCATACTAGAAAATCTAAGTGCCAGTTGGAAACCAGAATTAACATTTTGGTGTGTAACTTTCTGGCTGCTTTT
TCTATGCTAACAAACATATATGACATACAAAAATACACACATACACAAATTCCTGTTCACTACTTCTTTTATGTTAA
CATCACAATGTACCGTACACAGCTGTATTATTTTATATTTGATTTCATATTTTTTCTAAAGTCAGTGTATTTGTCAA
ATATCAACTTATCTATTTAATAGGAATATGGGATGATCTTTGCTTATACATACATACATATGTATATAAAAACAAAA
TCAAGTATTTTAAGCGTTCACCAGAAGTCATATGTCAATCAGTAAAGTATATAATTTTTTGCTGCCAATGACATATA
TCATAAAAACGCTACCTATCATAGAATGAAAATGAAACACAGCAATATTGGGACACCTATTCTCAAGCAACAGCTTT
GTGATTTATTAGCTATCTCACATGAAATAACTCATTAACTTGGTATTCCAAGCAGCAAAAGAAGGATCACTTAGGTC
ACTTGCAAAATAATACAAAGCTAGGTTTAGGGGTGGGTTGCGCTTGGTGGGATGTAGATGAAACCATATGGGCCCTT
GAGTTTATAATTGCTGGGATCTGCATGGTGGGTATATGGATGTTTATTACAGTATGCTAGTGAGTTAAGAAAGAAGA
GGAATTATTATTGACTTACATCATAGAGTTTATGCAAAAATTAAACGATAATTTATTTTTAAACTCTAGAGGTATAG
GTACCATCATGAAGGGACCCACAGAACTGATGTAGCCAGTAATTATTGGAGCTGGAACAGATACTCTGCTGTCAGTT
GTTCTGGTTTTGTGGTCATTGTTCTTGCCTTTGCAAGTTACCAACTCTAAGACCTTGGGCAATACTTTAAGTCTTGG
TTGTCTCATCTGTAAAATGGGGAGAGCAGTAAGTGTCTTAAAGGTTTATTCTCATGTTATATGACTTACGGTATGTA
AAACATCTGCGTTTAGACACATAGAGGGTGCTTAATGGATGATTGCTCTCATTATTAGGCTACATCTAATCTATGAA
TTTAAAAACTGTATAGAAATATGTGACAGATTCTTTAAGAGCCAAATACCAACTACAGTGAAAAATACTTAACACTT
GCTGAGCTCTTAGTATGTGTCAGGCTTAACTACCTTAATGCTCATAGCAATCCTATAAGATAGGTACTCTTGTTATC
CTATTTTATATCTTCTAAAATTGAAGCAAGGGAAGTTAAATAATAGGACAAAGATCATACGCTATCTATCCATATAT
ACCCATCTGGCTGTCTACCTGTCTCCTTCCATCCATCCATCCACTTATTCATCTACCCATCCATCCACTCAGTTACT
TCTCTCTCTCCCACCATCCCTTTCCCTTTCCCTCTCCCTCTCCCTGTCTCTGTCACTCTCCTTTACTTATCTATCTA
TCGATGGATCGGTTTATCTATCATCTATCTATCTCTATCATCTATGTATAGTTGTTAATAACACTAACATTTTATAA
ATTACAAGACTGAAAAATGTTTTCATTAACTTATGGTAACAAAAGACCACATTGTGAATAAAAAAAGCAGTAAACAC
AGGTCTCTGCACATATGAAAGAGATGTCCTAAACAGGAAGAGATGTCCTAAACAGTAGGGATACATAGTATCATACA
ATCAAAACATGGCAGCCCTATAAAACTTACAAAGCAATTTCATGTAAGTTATTTCATTTGACTCTTACCACAATCTA
TGAGGTTACTATTTTTATTTTTCTCATTTTACAGGTTAAATTTAATATGGCTTCCAATAAAAAATTAGTATGGTTAA
TAAATATCTTGACGTCTTGCTCCTATAATCCTACCGATAGTTTACAGTAATTAGTAAAATAAAATAATAGGAAAAAT
ACCTTTGATACTAGTATTAAATTATAATCATATCATTAGGTAATTTCAATTTGTGATTTTCAAGAATCTGTAATATG
GTAGCTTCTTCCTACTGACATGTTTGAATTCATTTTAAGGCTTATAATTCACAAGTAATCTATATATTATCTAAAAT
GTAAATGCACATTCACATGGAGATAATAAATTAGCGTGAAATGGCTGTATTTTGCTCTCTATAATTTTTAACATACA
GGAAATCACTGTTGTCTCAAAAATCAAGGAAATATAGTATTTGAGGTGAACTTATTCTTTCTACTATTAACACATTT
TAATATAGTTCTCTCACAGTGCAACAGAGCAAGAAGCTTTCAGACACATTTGCTGCTGCAAGGAGCATGCTGTGCTG
AACTTAAAACACCTTCCCTTTCAAACTCCTTGGGACTGTTTTTTTCCAAGAGACTTCAAATGCACTAAATTTAGCAT
CCGTTGGAGGCACACCCAGGCATATTATAGTGAAAGCCCCAATAACTGAATGTGTTACCACTATTCACAATGTTTAT
GTGTGTATATGCCTTATCTATGATGTATTGCAAATTACAAAAATTGTGTTATTATTCACAGTAACAAAAACACTTCC
AGCAAATTTCTAACAGTGATCTCTTTTGAAATAACTTACATACATGTGTCATGGGTCTTAAACTTTGTCACTTTTAT
GTTTCCATCATGTTGTTTTAGCCAGTGAGGGTTTTGTTTGGTTTTCATTTATGATTATATACTTTCAAAAAATAGAT
TTCAAAGTGTGAATTTGATTGATTGATTGACTGATTCATTGAGACGGTGTTTCACTCTTGTTGCCCAGGCTGGGGTG
CAATGGTGCGATCTCGGCTCACCACAACCTCTACCACCCAGGTTCAAGCGATTCTCCTGCCTCAGCCTCCCTAGTAG
CTGGGATTACAGATGTGCACCACCACGCCTGGCTAATTTTTTGTATTTTTAGTAGAGACAGGGGTTCACCATGTTGG
CCAGGCTGGTCTCGAACTCCTGACCTCAGGTGATCCACCCTCCTCAGCTTCCCAAAGCACTGGAATTCCAGACGTGA
GCCACCGCGCCCAGCCCGTGAATTTTATTTTTGAAAGACAAGAATGTCCTTGCCTAATTGCATAATAGTTTAACATC
ATGAAGACTAAATATGCTTTTTAGCCATGACAATTTTATTTATTATTGTTTTCATTTTTAATTTTCTCAAAGATCCT
CATCAGTGTACTCTTTTTGGTCTTCCTTATAAGCGTATTTTAACAGGACATAATAATAAGATAAATCCCAACTTTTT
AAAGTTGTATCCGTATGTATTACTTTAAAGTGCTATTAATATAAACGAATTAGAGGCAACTTTTATTCAATCAGATT
TTAAGTAATTTTACCAAAAATATGGCCTTGATAATGTCTCTGTAACAGGTTCTCTGTAATATACATGCTGAGGATTG
GTTTGTCTTTGCTTTTGATACTATTTTAATTAGAAAAGTAATGGGGAATCCAGACCCTTCTCATTTAATAATCCAGA
GAAAAATCAGTCCATGTTCTAATAGTTTAAATTTTTCTACTAAAACCCATGTGAGAATCCATATGAGTGGAATGGAG
AGGAGTTCAGCTTCAAAGTTGGCAGATTTGAGATGATTCTATGGCAACAGAAATGTGCTTGAGGGAAATCAGTTGCG
GCATCTTCTATAATTGTGTCACCTAGATTTTGCCTTAGGAATTTCTAGATTTCCATAGAACATTGTGACCTCAAATG
CTTTATCTTAATAAAGAAATAAAAGCAGATTAGAAGAATTATTTGCCTACAGTTTGTGGGAGATGGGCAAGTCTTAA
GAGTTTATTAGGTACCCAGAACGAAACATATTTTCTTGGGCCTCATAATCACATTGAAATACAAGGATTTAGTTATA
CACAGTGACCAGTTAGTGAATGACAGTCTTCAGTATCTAGTAGACAGTAAACATATAAAGATGTATTTGTGGCCGGG
CACGGTGGCTCACGCCTGTAATCCCAGCACTTTGGGAGGCCGAGGCGGGCGGATCACGAGGTCAGGAGACCGAGACC
ATCCTGGCTAACACGGTGAAACCTCGTCTCTACTAAAAAATACAAAAAAAAAAAAATTAGCCATGCGTGGTGGCGGG
CGCCTGTGGTCCCAGCTACTCGGGAGGTTAAGGCAGGAGAATGGCGTGAACCCGGGAGGCGGAGCTTGCAGTGAGCA
GAGGTCAGGCCACTGCACTCCAGCCTGGGCGACAGAGGGAGACTCCATCTCAAAAAGAAAAAAAAAAAAAATGTATT
TTTACTTTTAACTACAGCGAGAGACCCTGGCAGCCTACAGCATACAATTAGTGTTCATTATTTAGATTGCATGGATT
TAATGTGAGGGGTCAATTACTTGTCTAACCAGTGAGCCTAGCCTCTTGCTCAATACTGCCTGCTTCATGAGGGTGAA
CTGTGCTGGAGAAATATATTACAGGATTATCTGCAGATTTTTTTTAAATGAGTGGTTAAGTCAAAAGTTCTTGTGAA
AATTCAGAGTAATAAATTATTATGAAGTTGTGTAACTAGGTAAAGGATAGTTTCTTTTACACGGGTAAAGATTAACA
TGAGGAGGAAAACTTTAGCAATGGCATTTAATTCCATTCAATATATTTATATTGAGCTCCTTTAAAAATACAGGGCC
TTGTGGTGGGTGCTGAGGACAGAACAAAAACCAAGTAATACATGAACATAACCCTTGATTTCATGATCTAGTAGACC
TATAAAAGTTGTCGATATCTGATGAAAAGAAAATGGTAAAGATATTCCAAACAGTGTATGCAAATCCAGAGATAGGA
TGGAGGGGCTCTACCTGAAGGATGATGATAAGAAAACCGTGTTGAGTGAAGGGTGATTTGTGGAATTCAGATAAAAT
ATCAGTCTTGAATGCTGAGTGAATACTCAATGATTGACTAGATCCCATGGACAGTAATTTCTTCAATTATGACGATG
CTAGTGTTTATGACTATAACTATCATTCTCCATGCCAGGCACTTTGCCATTTGGTAAATGTATAGTGTGCTATTCTA
ACAAGCATGCACAGAGCTTTTACTTTAATGTATCCATGAGTTTATTGGGGTTCAGAATTTAGGTAAGCTTTGCAAGG
TCGTAGCATGGAGTAAAATATCTGAAATTCAGACCCATATCTAACTAAGTTCAAAGACTGTACAGATATTTCTCCTC
CTTTGTGCAGAGAAGGATAGGAATGGTTCCATATTATCATGGACTTAGTCAGATGTTTTAAAATTATAATGTCCTGT
GTTAATGAAGAAGGGATGATATTCAGTGCATATTCTTAACCGTTACTTTGCTTAATGCTCTCGACTTTTCTGTGAGA
TGGATAGTGTAGATAAAATCCCCAAGGGGACTCAGCAAGTGCAAGTAAAACAATGAAACTTTAAAGCCCTTTGTCAA
AACCTCTCTTTTTCTCAGAGGATGGAAGGGCCGTAAAGGTTGGTGAGGAAGGATGGACCATTTCCTATGTAGTCTTC
TGACAATATTCAAACAAAAGGAGAGTCAGCAAATCCCCCTTGATGTGGGAAGTTTTAATACAATTTGCAGAGTGTCT
CTCTGGAGTAGACATCCTCCTCTGCAATCGTGTCTTCTATATAGCCTCAGGGCTTTGGGTAGGTAATCCTCTCCAAG
GAGAGTCCTGGAGAGGGCTGTCTACCCCCCTTGCACCATCCTCTAACATTATTCTATAGCTCAGCTCCTTGTTTCTG
TTTCCTGCCTTGTTTTTGTCTGAGTCTGCAATTATGATGTAAGCACCATGAAGGAAGGTATGTTGCCAGTGTTTGCA
TCAGCATATCCCCCGTGTGTAGCAGCGCAAGGGATATAGTGAGCCCTCAATGTCTATTTGTAGAAAAAAGAATGAAC
GTATCAACGAAATCTGATACATATTCATTGTGTCTGTTATCTCCATCTCTCTTGTCCTGCCTTGTTATCTTGCCATT
TTCACAAAAGGCCCCAAGGCCCATCATTTCTTGTGTAACTTCCAGAGTGTTAATTTTTAAATTAAAATTAAGGCTTT
CTACATGAGTGTCTATTATTTGAGAAACCATGCAAGATCGTGTGTGTGTGTGTGTGTGTGTGTGTGTGTGTGTGTGT
GTGTGTGTTGCACTCTATATTATATTGAATTCTGGATTTTTTCTTATAAATAAAATTTTAAAAATAGTTCTTTAAAA
ATAGGAATAAGATGTTTTAGGAGGCACAGAGAGCAAAGGAGAATAAAAATTGCAGGTTTGGGGTTGTGCATACTAAT
TGCCATTGAGTAAAGAGAGCACACTGAGGCCATTTAGAAGAGAATTAACGTGTTTTGTTTTTGTTTTTGTTTTTGTT
TTTGTTTTTGTTTTTGTTTTGAGACGGAGTCTCGCTCTGTCACCCAGGCTGAAGTGCAGTGGTATGATCTCGGCTCA
CTGCAACCTCCACCTCCCGGGTTCAAGTGATTCTTGTGCCTCGGCCTCCCAAGCAGCTGGGATTAGAGGCGCCCACA
ACCACGCCAGGCTATTTGTTTTTTTTTTTTGTATTTTTAGTAGAGACGGGGTTTCACCATGTTGGCCAGGCTGGTCT
CGAACTCCTAGCCTCAAGTCATCCACCCGACTCAGCCTCCCAAAGTGTTGGGATTATAGGTGTGATCCACTGCACCT
GACCTTATTTTTATTCATTTAAAAATATTAAATGTTACTGCATAGGGAGTAATGGGCTTAACAATGAGGTGACCAAA
ACTCCTATGTACCATGCAGAGCAATGTATCAAATGTTTTTAACTATAAACTTCTCAAAAACATAAACCTAATTGTTC
TGCAGCTGCAGGTTATATCTGCCTTGTTTGAGCAAAATTTGGTGGTGAAAATGCCTTGCTTCCATTTTTCCTTCAAT
AACTGATATGGTTTGGCTGTGTCCCCACCCAAATCTCATCTTGAATTCTACTCCCATAATTCCTACTTGTTGTGGGA
GGGATCCAGTGGGAGGTCATTTGAATCATGGGGGCGGTTTCCCCCATACTGTTCTCATGGTACTGAATAAGTCTCAC
GAGATCTGTTGGTTTTATCAGGGGTTTCTGCTCTTGCGTCTCCACATTTTCTCTTCTGCTTCCATGTAAGAAGTGCC
TTTCACCTCCCACCATGATTCTGAGGCCTCCCCAGTCATGTGGAACTATAAGTTCAATTAAACTACTTTTTCTTCCC
AGTCTCAAGTATGTCTTTATCACAGCATGAAAACGGACTAATACAATAACCTATATAATTTTGAAAAGTACTTGTCT
AATAGACTTTCACAATAGAAACTATATCCTTATCAACTTTGAAAAGTCATTGCTTAATGCCTTTGGATAACTGAATT
TTCTAAGATTATTTTAATTTTGAAAGTTAAATTTTATCCCAGTGTTGACGATTTTTGTATGCTACTTTTAAAATATT
TTGTCAGTGATTTATATCTATGGTGCAATCTTGTAAAAAATTAACAATGCAAATGTGGCTAGACCATTTAAAAATCA
ATATGTTATAATTCAGCCCATTTAATCACTTTAGTTAAACATCTTAGGAACAACTCAGTTCCATTTGAGAGAAGACA
CAGTTTTCTAGATGTGTGTTGTGGCATCATATTGCTTTACAATATCTTACATAAGGTGAATTCAAATCATATCATTG
AATCTGTTTTAAATTCTGTCATAGCTTAAGATTAGTGACTAAATATTGGCAGGTTTATGGAAGTAGGATGTAAACAA
GACAAAAACAAGGGTGGAACAAGTAATTTTAGTATATTATTCACTTGCACAGAGAAAAGTCATTCACACCTTCTTCA
GCTTTGTGAAGAAAATAGACTAAAATCCTGTTGATATAGCAACTATGTTTTCCGTTTCTTGTATAAAAATAAAGAAA
ACTTCCTATTAGGAATTAGCCAGACATTTTAATTTTCTCTCTTCTTTCTCTATTTTCCCTTACAGTCTCTTTGAAGG
CAGGCAAAATTTCTATAAAGTTTTAAGAATGTTTTAAGATTTTTTTATTGTGAAATATTCATAGACTCACAAGGAGT
TGCAAAAACAGTACAGAGATTTCCTGTGTATACATAACCCAACTTTCCCCAGTTACATATTAACCAAATACAGTATA
TTACCAAACCCAATAAACTGACATTGGCACAGTGCAATCAACTAGACTGTAGACCTTACTTGGATTTCACCTGTTTT
TGCACATGCTCTTTTACTGTGAGTCATTATCTGTTATTCTATGACATTAACCATGTCTATAGATTTATATAGTTACT
ACCACTATCAAGATAAAGAAGTGTTTCATCACCACAAAGTAACTTAAAGGATTATTTTTATAAAGTAATGACAAATG
TGTCAAAAGCCATTCCTGTGTTATATAGCAAGTATGTTTTGAGTTATTAAAACTCACTGATCATGTCTTTCAGTGTC
ATAACTTTGGGTTTCCCTCCCTAACTATAATAATCCTGATGAATTACAGTTGATGAATATGAGAATATCCAACTCTT
CCTGACTCTATAAATATATTGACTGAGATTGTAATATTTATGGTGTCTTAAGGGGCGCTTGTTTTATTATGATGATG
TGAACATGTTGAGAATAGTAAGAACAGCCCAGTTTAGCAAACAGGATATGAGTCTTCTATATCCAGCTCAATCGTTG
CCCCAACAGGGGACATCTGCCTTTGCTACTTAATTTTCCATTCTGGAAAATGTGAAGTGTATGAGAATGAATAATCG
TCTCCGATTTTCCAGCACATAATAATCTGAGGAGAGCAGGTACAGCAATTTAGGAGCTGTTTTCTTTTGGTTTCCAA
AAAAAGTTCCGTCCAGTGGTCTAAGTTAGTCGTTTACTAAGTGATAGAGCAATTGGCTATGCTTTTTGAACGGACTG
ATAATTATGTGGATGCAGCAAATAGGATATAGACAATGCATCTACTCCATTACAGTAAAAAAGACTCTGATAGCAGT
TAATCCACATACCAGGCACTTAGCTTAGGCACAGTTGGAGGAAATGGAATGGTAATAGACTGTAGTATGGCATGACA
GGAGCTGTAGCTTGAGATTCAGAATTCCAACTCTGCCTCTCAATATTTGAGTCCTCATGGCCAAGATATGTAAAGTG
CTCTGTGCAGGTCTTGGCAACCATCCACCACACACTTAGTATGCAATATCTATCTTTATTAGTCAAGGATCTGGAAA
GCTAGTTGATGAGACAAATGATAGAAACAAGAGTTCATTAGATGAAATAAAGTAATAAATGATGCAAGAATTTAAAA
AAGATTTAGAGAAGGAAAGGGAACAGAACTCACATGCAAGTAGAGCAACTGTGTATCAGATAATGTGCTAGCTGAGT
TAGAAACCATGTCTCATATTACCCTGAAAATAATTCTGCAAAGCTGTAGGTGTTATTTTTTTCATTTGACAGGTGAA
TTCATGAAGGCTTGAATATAGGGTTAAGTGAGTTGTTTCAATGTAGTTATTGATTCAAATCAAGATCTGAATGACTC
TAAATATGGTGCTATAGAGATTTGAAGTAGGATAAATAGGATTTGAAAAAAAGAAAAAATATATAGGGAAAGGAATT
GGTACACTGTAGCAGTGTCATAAATGAAGCTTCAGTTGTGTGATTCCAGATGATGTATGTGAGGCCTAATCAAACAG
CTTTGTGGAATCAAAATTTCTGCTCTTGTCTCCAACTGGGGACGAGTTGGCTCGGGATTAAGGTGGGCGACCTTGGG
AAGACTAGAGTCTAAGCAGGACTTTAGTCCCTCATAAGAATTATATGAGGATGTATATTTGCATACAAATTCCTGGG
CCCACCGAGATCTGCCAAATTGGAATGTGTGGTGATATCACCCAGGGAAACATAGAGAGCTGTTATAATTAGTCATG
AAATATTTAGTACTGAAATTATAGATTATGTTAAATAATCACTTATAGGGGACATAGCAGGGTTGGCAGGTTAACCA
TACAGCAAACAGGGTTGTAAGTCAGGGCCTAGAGAATTTTCAAGAGGCAGGAATTCTGCAGAATGAAGGCCTGGTCT
CATGCAGCACCATGGACAGCTCCGAGGCACTCTTGTTTCTCCAAAAACCTGAAATCAAAAACTTTGCTTTTCATCAT
GCAACATACCCATGTAACAATCCTGCATAGGTACTCCCTAGTCCAAAATTAAAGTTGAAAAAAAAAACTATACTTTC
ATTTGAATACAGTTCTCTTCGGCTTTACCAGCTCTACTCTGGAAGGAATATCTTTTACTCAATGAAAGGCCATCCCC
TGTTAATGCCTGGCCAGGTTCTCCTTATCAGTCATTCACTATCTTTGTGTGTGAGTGACTAAACATATAATGCTATG
TTTAGTGGATGGAGTAAGATTACCTTTGCAGAGGTTGTACTGGCTTACCCCTTTGGTTCTTGTAGTTTTCTTCTATT
AGAGTTTTTTCCATCCCTAGGTTTCTATACTGTTCAAATGGGTTTAAGATTCTTGAAGGTATTCCTCTGACCTTGTA
ATTTATGCTTGTCTCCTAGCACAACTTTTTTTTGTAAAGGAGGCACCAACTATGTGGTTTGCTGGCGATGGCATACA
CAAATCAGGTGGGAGGAATTAATGAGAGCAGCAATTCCAATATCTGGTTCTTCAAGATTAACTTGTATAGTTTAATT
CAGCATTCTAAATAAGCCTCATAGATTTAAAAATCTAGAATAAACCCACATTTTTAAAAAAAGTTTTATGTTATCTG
TGCTGATAATGCACGCTGTACATAATAAAATATTATTTTCTTTTTTTTAAATTTATTATTATACTTTAAGTTTTAGG
GCACATGTGCACAATGTGCAGGTTAGTTACATATGTATACATGTGCCATGCTGGTGCGCTGCACCCACTAACTCGTC
ATCTAGCTTAAGGTAAATCTCCCAATGCTATCCCTCCCCCTTCCCCCCACCCCACAGCAGTCCCCAGAGTGTGATAT
TCCCCTTCCTGTGTCCATGTGTTCTCATTGTTCAATTCCCAATATTATTTTCTAAGTGGCAGTGGAAGAAACATGGA
AAGTTCTACTTCATCCATCGGTGGATTAGAATTTGTATACCATGAGATGATTAATTTTCAAAACCAGTTTGAATCTC
ACAAAATAATGACCCTGTTTTTTGAAGGACAAGGCAGAACAAGGAACTAGGCTGTGCCACGTTCAAGTCACAATCTC
TAACATTTGTTTTGTTTTGTTTTGTTTTGTTTTGTTTTGTTTTGTTTTGTTTAATCTCTGTTGCTTGTTCACTTTCT
CTTGTAATCTGCATTGATTTGCTACCTGGCTATTTGTAGATTGACTTCGGCTGCCAGGAATGGAATGTTTTTCATAA
AGGAACATATGCCTTAATGAAAGTACCATAAGAAGGGAGTAGAGTGTGACCAATTGCCTAGGTAATAAGTAGTGACA
ACAATGATATTATTCTAGTATAAATGGAATCAGTTTTTCTTTGCCCAGGGGGCATGATAAAGAAGGCCTGGCTGGTA
TATACTAGGTGGGACACACCAACAGTGCCTAGAATGTCAATGGATCAAACCTGAGGGAACCAGAAGTTGAAAAGACA
TATCCCAAAAGAAAGCATTTGATGTTTAAGGGTTGGCTTACTTAGAACACAATGAAAAATATTACTAAAATTAAAAC
TATGATTTTAGCTATTTTTAAATATGACAAATTAAATAGCAGAACATTTTAATAAAACATTACTTAAGGTCCACAAT
TTTCTGTAAGTCTAATACATGGGTCATTAAAATAAAAAATTCCCCATGATTTATGGAATCAGATTTTTTTAATACAA
CGAATTCTAAATGGTTTTATAATGCCAATTCCAATTAATATCCTAATTATAACATGTCATCCAGAAGGGTTAATGAC
TAAATTTTATTAATATTTGTTTTCTATTTATTTTGATTTGTGCAGTTTATGTGTATAGTAACGATAGCTGCAAATTA
GATACCATTAGCATTAAATAAGGTATATATTTTAATAGAAAATTAAAGTTAAGTATTTGAGCTAGCCTAAAATATTC
AACAACTTAAATTTGTTTTTTGTGGATCACATTTTTTTGAGACAAAGTCTTGCTCTGCTGCCCAGGCTTGAGTGCAG
TGGTGCATTCATGGCTCACTGCCTCAACCATCCAGGCTCAGGTGATCCTCCCACCTCAGCCTCCCGGGTAGCTGAGG
CTACTGGCGCACGCCACCATGCCCAGCTAATTTTTTGTATTTTTTTTTAGAGATGGTGTTTCACCATCTGGTCTCAA
ACTCCTGAGCTCAAGCAATCTGCCCACCTTGGCCTCCCAAAATGCCGAGATTACAGGCGTGATCCAGTGCACTCACC
CCTGTGAACCACCATTAAATAGCTAATAAAAGATGCATGTCAATAAAAATAAACAACTTACTAGAATGATTATGTGA
AAATCATTTATTCTTCCAAAGCATGAATTTTCAAACACACCTTTTGTTACTGTTTTAAGAAGGGAATCATTTCCATA
TATTTGCATGTAAATCACTTTTAGTCTCAGAGAACTTTCCATAAAAGTTTTTTTATTACTGCTGTAACCGATAGAGC
TAGTGGACTATTAATTTAAAAAGCTGTACATAAAAACACATCTATAGCTCAAATAATCTAGGATACCTTTTAGTTTG
GGGAAATGTAGATGAAAATGAAGTAATTACAGAATCCTTGTTAATTTTCAGATTTAGACAGTCTAGGCAATATCTTT
CAGGAATGAAGAGATATGTGTTTTTTGGCATCTTGGTAGAGTATATTCCCATTGTAATTCTTTTGTGAAGTCTAGAC
CAGATGTGGCCATAAAAATAGACCCCTACTACAATAATATATTTCATAGATAATCCAATAAAGTCAAATCTTATTGC
AGTAGGCTTAGAACTCTGTTTGCACCCATGGAATTTATATCAGTTTTTGGCAAATCCTTTCATCTCTGAGGATACTT
TTTCATCTCACATATACCCTATTTTCTGAACATTTTGCCTTCAAAGTATACCTCATTTATCAAGAATTTCTCTTTAT
TCATCTGACTTATACAAGTGGCAATAACAACGTCTGGTTCCCATGAAGTAACCAGTGACCCTTTGAAATAATATAGC
GCTGGAAGAAAGAAAAGGAAAGGGAGACTGATCATTCAGCAACTCTTTAAAACCATGTCACCGTTAAACACATAGTT
TATTTTATCTTTTTTTTAGAATTGTGAAAACCTATATTAGCATCTTCACGGATGTCTCCTTTGTTTACATCCCCGCT
TCTGTGCCTTGCCTGCAGTAGAAAAAAAAAGGACATGTGTATCCCTATTCCCCATTGTCTTCTCATTCTACATGAGA
ATGAGAATTCTTTTAATTTCTTCTCTATCTACATGAACCCACTTCCATTATCTGTTTGTTCAGTTCTTTAAATGCCC
TGAAGCTAGCTCTGTGACTGGGCAGTTGAAAGTTCTGGACTTAGCATCAGGTTAATTTGAAAAATACTTATTGAGCC
ACCACCATATGTCAGCCACTACTGTAGATGTTTTGAATGTGTCAGTGAACAAAGCAGAAAAGATGTATGCCCTCTGG
ATTCTTGGGGGTCTCAAATAGTGAAAGACAGATACGATAAGTATATTGTATAGTATGTTCAAAAGTGATAAGTGCTG
TGAAAAAAAAGAAGAAGGGTAAAATAAGAGATGGCTCATGCTGGAGTACATTCCAATTTTAAATAGGGTATCATGGT
ATTCTTCATTGAGAAGGTGACATTTGAGCAAAGATCTCAAAGAATGAGGCATGGGGTTGAATCATGTAGATATCAGC
AGTAAACTCATTTTGGGTTCAGTAAACAGTCAATGCAGAATTCCTAAGCCATCGGTTTATCTGCTGTTTGGGGCTGG
TTATCTGCAGTGTGGCTAGAGTGAAGTAAGTGAGAGAGGTTTAGGAGAGAATGTTAGTGAGGTGAGGGTGGACCTTT
GAAGCCATTGTAAGGACGTTTTTCTCTTTCTAAGTGTGAGAAGATGATGCTGACTGAGACCAGGGTGATAAGAAATA
GTCATATTCTGAACGTGTTTGGAAGTGGGGCCAACAAGGATTTCTGGATGAATTGGATAAGGGGCATGGGAGAAAAA
TGGAGTCATGAATGGCTCCAACGTTTTTGCTCTGATTAACTGGAAGGGATAAAGTTGCCCTAAACTGAAATAATAAA
GACTATAGATAGAATGGGGCGATTAGGGAGGCATTAAATTTGGATATCTGTTAGACATATCACCAGATATATTGAAT
AGGCAATTGAATAAATACCTTTAGAGTTCAGCAAAAAAGGTCCAGGTTGGACGTTTAAATTTCGGAGGTGTTTGTAT
AAGATAACATTTAAAGCTGTGATATCAGATTGTATCACTAAGGAAGAATATAGATAGAAATGACAACGTGACTAAGG
ACTCTAACATTAAGAGGTGGATTGACAAAGGAGAAAACAGCACAGGATAATGAAAAGGAATGATCAGCCAGGCATGG
TGGCTCACACCTGTAATCCTAGTACTTTGGGAGGCCGAGGCGGGCAGATCACGAGGTCAGGAGTTGGAGACCAGCCT
GGCCAACATGGTGAAACCCCATCTCTACTAAAAATACAAAATTAGCTGGGTGTGGTGGCACACACCTGTAGTCCCAG
CTGCTCTGGAGGCTGAGGCAGAAGAATTGCTTGTACCTAGGAGACAGAGGTTGCAGTGAGCCAAAAGATTGTGCCAC
TGCGCTCCAACCTGGGCGATGGAGCGAGACTTCATCTAAAAAAAAAAAAAAAAAAAAAAAAAAAAAAAAGAAAAGAA
AAGGAGTGATCAATGAGATGGGAAGAAAAACAAAAGTGTGTGGTGTCCTCAAAAACTGACGTTCTATTTTCAAAACC
TACATTTTGGGTCTCCTTTTACTATATCCTGACTTTCTAGCTATATAACCAAAAGGAGAAAGCAGTAATTTTTTTAG
ATATAACATGTTAATAACTCTAAGGGTATTCAATGAATCTGAATAATTCAGTGGTATAATGTGAAAAAATATAGTAT
TCATAGGAAAAGGAACAGAAGTTAGCTCAGGAAATGACTTGAATGAACACCGAAGCCAAATCTCCAGCGCAGGTCCA
CGTATTATTTGTCTCAGTGGTTGAATTAGCAGCAAGATTCCTTAGTAGGATGAAAAAAGATGTTGTGAGCATCTGTA
TCTACATGACTGAATTAAATTCCTCCAACAATGAAATGTAGTTAACGTAGTATCTCGAAAAGAACCCTAAGTGGAAT
TCAGGGAACCTAAATTCCAACCATGGTTTTGCTGCTGACTGATTGCATTCACTTCAAATCTATCATTAACCTCCTTG
TGCCTCATTATCCTCATTTCACCAAATAAGAAAAATGAAATATTCCTCCTTCCCTACCTCACTAGGATGTTGTGGAT
TTAAATGTGTGAGAAGTGCTTGAGATGCATAAAATTTGATGGAGTGTTTTATTCATGAATTCAAGGCATCTGAAGTA
ATTTGACCATGATGGACAGTTGCTTCCTTGCACATTTTTTAGAGTGACATTTCCGTTACTGACCCACCCATTTATGC
AACATGTTGCCTAATCTAAATTTAGGTCAAAACAAATTGACCTTATAGGTAAGCATTATATCTATTAATATTGTATT
TTTGTATTATTTTATAATATTCATCATTCACCTATTTTCTCATGCAATATATGTTACTGAACACATATAGATTAAAA
AGCCTTCATCCCTAAATAACAATGATGGGACCTTCCATTTTTATATCCCTCTGGCATTTAAAATGTGCTTTTATAGC
CATCATCTCCATTGATCTCTCAGTCCCTTGAGGTTGATATGACAGATATGCTTTTTCCATTTTAAAATTACGGAACT
GACAGTCTCAGATGACTTTACCCTCCAACTACTGTGTGAAGAAGCAGGGTCTGGCACTGAGGTCTTCTGACATCCAG
TGTAGAGCACTATACTTCACAATATGGCCATTGGCTTACTTTATTACAAGCACTAAATATTTTCCACTGAATACGTA
ATACCTAGAGGAGAATGTCGTGTAAAACAGCAGCAGTAGAACAGAGGATTAAATGACCCATTTTCTTGAAGTTATCT
TAGTTTTAAAGGGTTTTTTCTTCATCACTAATGACCATCCCTGACTAAGAAATTATTCTCATAATACATGATAATAT
CTGCGTTTTCCAATGCGACAAGAATGTTAGGATGTCTATACATGATCTTGACAATCCCTAGCTCCATCACAATGTGT
CCAAATTCATTTTATTTGGCTAGACAGGCATGTAGTCTTACTTTCAATGGTTGGCTCTGCTGGATGCTATGTGATCT
AGAACCTGTCACTTACCCCTTCTAAACTTCAGGAATTTTTTATCCTTAAGATAACAAGAAAACTCGTACCTGTTTCA
AAGAGCTGTTTGTTCAATCACCTATCCATTGATTATCTTCTATATGCCAAATGTTTTTCTAGGTGCTGAATTACAGG
AATGAATCAGAAGCAAAAAGTTCTTACTCTCAAGGATCTTATATGCTAATGAAATAGATGTTAAAAAATAACAATTT
TTGTTTCATTTTATTTTATTTTATTTTGTTGAGACAGAGTCTCACTCTGTCACCCAGGCTGGAGTGCAGTGGCACAG
TCTCTGCTCACTGCAACCTCTGCCTCCCGGGTTCAAATGATTCTCCTGCCTCAGCCTACCGAGTAGCTGGGATTACA
GGCATGCGCCACCATGCCTGGCTAATTTTTGTATTTTTAGTAGAGATGGGGTTTCACCATATTGGCCAGGCTGTTCT
CGAGCTCCTGACCTCAGGTGATCTGCCCTCCACGGCCTCCCAAAGTGCTGGGATTACGGGCATGAGCCAGCGCACCT
GGCATTAAAAAGTAATAACAATTTTTAAATATCAATATGTCTTATACAGAAAAGTGAGCAGTGTGGTAGAGTGTAAC
TGGAATGTGAGTTGAGACATAACACCAGACAGAGAAGCCAGAGAAGGACTTTTGTTTGAGGAAATGACATTTGAAAA
GGAACCTGAATAGTGACAGAGGCAGATACCTAAAGAATATGTTCCAGACAAAGGAAACAAAAAGCGTGCAATTGCAT
AGTCAACTTAGCCTACTTGAGGAAAAGTGTGAGTGGATTTTGGTGATGGAGAGGTAAGTGCCAGGAGATGAAGGGAG
AGATCTGGCATGCATCAGATGATGTGCAGTCTTCCGGGACGTTGTAAAGAGTTGGGCTTTTTTTGTTTATAAATTAA
ATGTTAAGCCATTGGGGTTTTTAACCAGAGGAGTTATGTGATATGATCTATAGTTAAATTATGTTTGTTCTTGGATG
GAGTGTGCATTATGGGAATTTATACAGAAACAAGATTTCACATATATATATATATAAAACTCAGTGTCAATAGAAAA
TAATAAAAACAAATTTTATCCATTGATAATTCTGGCATTGATAGTAGTGGGTATGGTGGTAATAATTGTGTGTAACA
CTCAAACTTTCTGAAAACCTACACTTGATCTGTAAATCCAAAAGTATATGTAGCAAAAGCCATAATCTGCTCTTATT
TCTGCACCACTTGCACCAGTGTGGAGTGATAAGGCAAATTATTCAGGCACCTGTGTAAGCCTTCAGTGTCCTCACCC
CCTTGTTATAACTCTCCACTAATATTACATTGGTAAAGACGTCCCTGACCTATATGTCACTGAGACCTCAAAGAAAA
GAGCAAAGCTAAAGCGTAAGGGGGAAAAAAGCCAGCTTAAAAAGACTTAAAGGTTTCTGGGACCAAAAAAAAAAAAA
AAAAGTCTTTGAAAAATGAGAAAGGAAGGATAGAAGAAAAGATTCTCCTTTGGTCAATCTGGCCAACCTTTGGAAAT
AAAAAGTATTGTGTTGCAGCTAATAACTATTTGTCACTGCAGGCACTTGCTGATGTCTGCCCTTTAAAATGACCCAA
ACTCGTTGGCCTCGAAATCAGAAGCCAAGGAAAAAATCTTGGACATAATGTTTTCTGTAGAATTACCAATTTTCTCT
CTCTCTCTCTCTCTCTCTCTCTCCCTCCCTCTCTCTCTCTCTCTCCATATCTATATATATATAGATGTATATATATT
TTTTCTGTAGGAACTACCAATTCCTATCTATAGGGACTGATTGAGAAGTCCCTTATAGCAGTTTTTCTTTGGCTTTT
AGGATGCAATGATTATTGGTGAGAATAACTCTTTCATTTCACATTTGTCATTGGCTTATTTGAATGTAATCCTGATT
CAATCGTTATGATCTCCTTTAAGTAGGAAGAGAAGCTGGTATTACATTGTAGGATTTTAATTTTGTACTCATGAAAC
TTTTGAAAAACATTACTCATACTCTTCTGACTGTCAAATTGGCCTCTAAGAGGTCCACATCTCAAGAGGTATCAAGC
ATTGGTAACTATTTTTTGGTGTTGTTTTCTCATCATAAAATGTACTTTTATTAGGTGACTTTGGAAATTTTATTGAA
TCAATGCATGACACTGCCTCATTCTAGTAATCTGATGAAGCAAAGCTGAAAAACAAAATTTGAGGATTGTCAGTATA
TATACTTTTATTTGCAGTCAAGAGTTATGCTGCAAAAATGGTTTATTGAAGTAACAAAATTTTAGCTGATATATTAA
TCTGAAAGATACAGTATACATTTTTAGTATGGAAAAGATGAGGAAAAGGAGGTTCTCTTTCCTCTAGGTATCTAGAG
CAAACTGTAACTGTCCTTGGTATTTAATTTTTGGCTAAGGTACTGAGATTAGAGGTGGGGCCTTAGATATGATTAAT
TGTCAGACTGATAAGCTAGATATTTCATTGAGTTTCTGTTGTGCTCTTTCTTTCAGATCCTCTGTTCGATGCTTTGT
TATAAAGATTTGGGCATTTCAAAATCTTCTCCATATCTGGTGTCTTTCCAAACAGCAGGTCATAGACTTTACACAAA
GAGGAACGACACAGGTTATAAGTAGAAGTGTTTTAAACCCTGAGTTCCTATTTCAGTTTTGCTTTCTTAAACATATT
TTCCTTATGTGATAAATGCGAGTGTTGAATGGTGATAAATACCACCCATAGGCTTTAAAGCCTAAATGTTGAATTTG
ACACTGAGAGTTTAAAGGCATCATGAAAATTTCTCCAGAACTAATGTTCAAGCAATTTAGGTTTTACAGGCAACTCA
ATAGTTTTGAATGATGTAGTTATTTTGAAAAAGTCACCATAAAACGCTATGTTTAGGGAATTGGTACTTTGCATTTA
TCAGAAGATTGTAAATGTCAATCGATTGGCTTGCTATTTGGAATATAATTTTTTAAATTATAGTTCAAATCATTAGG
ATTTAATTCATGATTTTGTACTACAAACTAAATCTATGAAAAATATCAGATATTTATTTTAAATTAGAGGCATGTAA
AGGAAAATATAAATTTTGAAATGCCATTTTACTGGATTTTTCTCTTCAGCCCACCCTAGGCATTTGTTACATAAAAT
ATTTCTGAGGAAGTCTTCCACTGATTTTGTAAACAAACATGTTTTATTGAACAGTTCTTTGTTGACTAGATTAACAT
TGACCATTGTATGCAATGCATTCTCAAAATCTTAGAAGCTGGTTTTCTTTTTAATCATATAATTTTACTTGTTTTAC
AGTGAAATTAATGCATGTAAAAAGTATACCTATATAGAAAGTTAAAAGAATATTGCTAACTAGTTACTATACTTCCA
AATTGCCTATTTTCTGTGTCTTGCATTGGACAGTAGTGATTACCTCTAAAAGAAAATGGATGGTCTTTGTTTCATTG
AAGGGATGGATAATGGACATAACTGGCATTCTTGAGCAATGCAATTGCAAATACATGTCTTTGCATTTATGGTCCAA
TCATCTTCTTACTATGATAGCATATAATTGAAGGTTCAAATAAATGCCTCGTCCCTTCCTGTGGCATATTAAAGAGA
AAGAAAAATTAGAAATACTTTCAAAGCTACCTCACATACTAATGGTAGAGTTGTTTGAGTATTTAGGTGATTTAACA
AAGCTGATGTATTTTATTATGCTTGATCATTGAGGAAAATTTATTTATCGGAATGCTTTTGAGAGCATATATATTGT
CAGAGATAAACACAGCTGGATATTAAAGAGGTAAAAACAGATTTTATTCAATACCTCGTGAAATTAGGGGAGAGCTG
AGATCCATTCTAATTTGTGCAGAGGCGACTTGGTTGTTTTAAGGCAAGAAGGAGGGAGAAGGAGTGGGGGTTCATTC
GAGTTAGAGAAGTAAAAAAGTACAAAGGGCTGGACAGTGTAAATGTGATTAGGCCAGCTGTGTTAGCTGGAAGTTAT
TGAAGTTAGGATTCTATCTTCCCACAGAGAACAGGAGACAGAGGACTTATCCTTCTTGATGATGTCATTTGAAAAGA
ATGGCTTTCAGGTCCTTGAGTGAGAGACACTTCTGATTTCCAAGAGCTACATGTTCACAATTGTAAGCCCTTTTGAG
TAAATGTTCTAAGAAACGGAGGTAAGAGTCCTATCAACAGATGTGTGTTGGCTAGAACAAACATTAAATTTTCCTGG
CAGCACTGAGCTTTCTCAAGCAGGCACTTAAGGGAAGGCTAGGGTCATCCTAGGGACATGGCCTTCTGGGGCTAGAA
ACCATACTAGAGTTTAGTCAAGTCTTAGTGCAAGGGTTTGGACAGAGTTGTTAAGTGCTGAGAGTTCTGTATTTCTC
ACTGTCACAAAGGAAGATCAGAAGCTCCTGATACTTTTTTCATCAGTACAATTGAATATATAAATCCTATACACAAA
AATAAACTAAGCTTATACAAGCATATTGGTCAAGGAATGTTGCTGGCCTTATTAATTAGATAGCCCAGTTAAAAGAA
GAATTTTTTAATATAATTAATGTTAAAGTAGGATGATAGTATATAAAACGTGTCTACTGTCCTGAATACAAACTAAA
CTGTTTGGTTTAGCATTTACCTCAAGATCTCTTAATATCCCCCAAAGGGTCCCTAAAACCACAACTTATCTTTGTGC
TCATGAAGTAGAGAAGAGACAGTTAATAGACATTTCTAGCTGATAGACTGTTGTAGAGCAGAGAACGCTCTGTGTTT
TTGAAAATTAAACATATGAATTTGCCCCTCTTCCCCTATTAAGGAAGAAGAGTTTCTTAATTGTGCGAACACATCAA
GTGAACTATTCAATTAGATTTTTGTGACCCAGGGTATAAACATCTGGTTAAGGTTACATATTTCAAAGGAACAAAAC
ACTAGAAACTCTTGGTTTTAAATCTCATGGCTGGAGGATAATTTGCAGCAGAGATTTATCTGGCAAGCATACAGAAT
TGCTGAGACTGTTCTAAAGATGTAAGTGTGGGTGTTTGTGTCGTGAAAATAGCTGTTTACATCTATTAAGTGGATAC
CGATGGTTGAAAGTGCCGTCTATGTCAAGTTTTTACCAAATCAACTTTTGCCTCACTGTGTCAGACCATTTTACCTA
ATCAACTTGGACTGCTAATGTCCTTTCCCCTGGCACCACTATCTGTCTCTTTTGCAAAGCACAGAAACGGCATGCAT
GATTGTAGTTTATAAAACACATGTACCAATGTGGTCTACAGCTTCTGTTGAGTTCGAGAGGGTCAGTTTCTGTAATC
TCTTCTGGCACAGAGTCAAGAACAGCTTCACTTTCCTCCTGCTACCTCTCTACCCGTAAGTGTGAACCCATCACTTT
GCTAACACTCAGGAAGGGGATTACACAAAATAGAGCAGGAGCCCTCTGACCTGAATATGCATCTGAGCCCTAGCCAT
AGAGCTTCTGATTCAGTAGATCTGGGATGGGGCCTAAATATTTGCATTTTTAAGTGTATAAGTGATGCTGATGCTGC
TGGTTCCAGGACCACATTTTAAGAAATATCGATAAAGGTGGAGAATTAAACTGCAGCTCAGAAGACCTGAGTTCTTG
CCCCAGCTTGACTTTTACAATCTAGCAAATGGATAAAACTCGCAGGACTTCAGTTCTCTTCATCTACACAGTGAGTG
GTTAGATTGGCTTTGTAATTTAAAATTAAACAGGGTTTGATTCTGATTCACTACACAAGGTTCCAAAGAAGGAATGA
TATCTCCTTTCATTTCTTCACTTTGTCTTCTGTCCCTAGGTAATCTTATCTATGTTCCTGATTTAACCTAACTAATG
TTTCTGCAAAGCTTCTAATATTTACATCTCCAGCCCTGAAACTCTCATTTGAATGCTAGTCTTATATACATACCCCC
CTGCCTAATTGACATCTCCACTTAAATGTATCAGAGGCAACTCAGACTCAACAAGGACCAAACTGAATGTTCGACCT
TGTCCTTCAAACCCGATACACATCCAGGTTCCTCCATCCCAGTGAATGACACTATCCAGTTAAGCAAGCCAAAAGTC
TGGATTTTTTTTCCTCACTCTTCCTCACTGTCCGTCAACTACCATTATTAAATCTGTCACCTGGTCCTACTGATTTA
ACCTTCTCAATATCTCTACAGTTTTTCTTTATGCCCATTAGTATCCTAGTGCAAGCTACCATCGTCTCTCATTGGAA
TTAACACAGTAACCCCCCTACCCACCAGACTGTTCTGCCTACAGATAGTGTGATATTTAATAAATATAAATCTAGCC
TTGGCTAGATTTCTCCTTCAAAAGGTTCACATTAATTTTAGCCTTAAAATGGTGTGCAAAGCTTTGCATAGTCTGTC
CTTTGCTATGTTGGCAGTATTTTTTACTATCCCTCTCATCTGCTCATTCTCTGTACTCCAACTACACTAACTTTGTT
TTTTTTTTTTTTTAGATTTCTCTAACTACAGTGCTGTAATCTCTTTTTCCTTTGCACGTACTATTCCGTTTGTCAGG
GAATCTGCTCACTGTCTCCACCCACTCCACACACTCACGTTTTCCTGCCCGTCTTACCGGTCTTGATCGGTTGTCAC
TTGCTCAGGAAGGTTTCCCTGGTCACCCCCTCCACAAATTGAATTAAGTCCTCTTGCTGCATGCTGTCCTAGTGCTC
TTTATTTTCCTCTCCTCATCCTTAATTCAGTTTGTAATTACATGTTATTTGTGTGAGGATTTGATTATTATCTGTGT
CACCCACTAGATATTGGGCATTCTTTACTTACTCACCACTGAATTCATAGAACCACAGTAATTGTACACAACAAATA
TTCAAGAGAAATTTATTGAATTGATGAATGAAAAGTTGTACCTTAACATGTTCCTGACATGTATCCAAAAAAGAGCT
CCCCTTTGGGGTCTATTAGGACTTTGGACCTAGGTAAACGTAACCCTAGTTTCGCTCAGGTTTAAACAGTAGAAAGT
AATTGGGTCTCTTTTGCATGTGGCTTTCCTAAGGGCTAACCCTGTCTTCGGAATGAGTCAATACAGCAGAGCTGTTG
AAAGCAGACTCTAGCTTCGGACAACGTTGGTCCGAATCATGGTTCCGTCATTTCTTAGCTGTGTGATTTAGAATAAA
TTAATGTTTTAAAGCTTTGATTTCCTCTTCCTTAATCTGGAGATGCTAATAAAGCCAACTTCGTAGAGGTATTGCGA
TGAGTAAATAAGCATAATTTGCTGTAAACACCTTGCAGATTGCCTGTTGTATGCTAACTAATCAATAAATTGAAGCT
CTTAACATCATTATATTAGATATTTCCAGCATTGAGTATACTATCAGGCATGTGGTAGAAGCTCAATATAAAGTTTT
GTTAAATTGAATAGATTCCATATATGGTATTTCTACAGCATTATGCTCCTTATTTAAGTGTCTCTAAGTATTTTTTA
AGTATCACCTCACAAAAGACAGATGTTTAATTCATTACACATGTGAATTGTTTTAGATAGAAAATAAAATAAAAAAT
TCAAACATTGAAATCAATAGTGTACCTTACCTTAGGATTACACCATAAAATTTCTACCAATCGAGAATAAAGTGTAC
AGTCTATTTCCTTTCTAATACTTTTAACGCAACAAATGTTTATTGAACACTTACTACTTCTAATCTATGACAGACAT
AAAGATGAATAAAGCATGCCACAATGTTTAAAGGAGCTCACTATATCATAAGAAAGCGGATTCACACAGACAACTCT
ATAAGATAAAGTGGTAAATTTAGGCTGGCCTGTGAAACAAAGGATTATAGGTATAGTTAAGAGGTGGAATTTATTTT
ACTTCGAGGATTTCAGTTACCTTTATATTCTTTGTCTAACCTTTCATGTTTCTCTTTCTTCAGAAACAGAGCACCTT
TTTCCTGACACATTCATTTCCCCCTATGGAGTAGAGCAGTTGTTTTCAAAGTGTGGGTCCCAGATCAGCATCACGGG
GATGGTTAGAAATGCCCATTCTTGAGCCTCACAACAGACCTACTGAAACAGAAATTCTTGGAGAGTGGAGCCCGCAG
ATCTGTGATCAAGCCCTGTAGGCAATTCTAACGCACACTCAAGTTAAAGAACCACGGGAAGAAAGGTCCATCCTGTA
ACAAGACAGATTTTTTTCATTAGCATCAATTTTGATCATTTATATATATATATATATATATATATATATATATATAT
ATATGCATGCTCACAAAACCATTCACCTTACTAGGTTTTAGTATTCCCCTTCCTGTATTCATGTGGTATGTATGTAT
ACAAGATGAACACACATTTACCTGAGACAAGGTAAGACTACACATGTCTCATTTGGGGACCAGAGGCTGTAATCTTA
CTCAAGGTCAAAGCGTCTTCACTGCTTTCTTTCACTGCTTTTCAAAAGTAAAATTTCCATGTAGGTGTCATTTGTTT
TCTTTTTGTGTTTTAGAAAACCGATTAAGGGGTGAAGTCTGGCTAAACTTAGTGTCAGGACATTTACTTAGATAAAA
TTATTTTAATTTATCTTGTAATGTTCAATGTGAGAAGAAAAGTCCTTATGAGTAGTGTATTCCTTAAATAACAACAA
TTTAAAAACTACCACTGAAGTCTGTCAGAGTAGTTTTGCCTCATTTGTCTAGATAAGAGAAAAAAGGTTCACATTAG
GGATTGCAATTTGTCTGCCAAAGTGCAGTTTATTTATTCAGAAACATTTAGAGAGGAATGTGTCAGTTCTGTTGCAG
GCACTGTGCTGTGACGGGGAGCTCAAGATGATCTCAAAAAATTTCACAGATGGGGTGGGCAGGGGGCACAGAGAGAT
GTATTTAGTGGTTCAGATACTATTTAGACTGTGGCCAGCATTTCTCTAAATGCAATCCAGATAACACCTTACAGAAT
CATCTGGGCAGCTTGATAAAAGCTGTAGACTCCTACCCTTCATCCCAAACCTATTGAATCAGTGTCTGTGTGTGAAG
ACCTAGATTGTGACTGGTAATTATACCAAAGTCTTAGAAGCAACTCTAGGCCAGTAATACTCACATCAGAATCAGCT
GGAGGGTTTGCTATACCACAGATTGCTAGGTTAGCCTTCAGAGTTGCTGGTCCAGTAACTTTGGTGCAGGTCCAGAT
TTTGCATTTCCAGCAAGTTACCAGGTGATACTGATGCTGCTGGCCTTGATCGTGCTTTGAAAACCACTGCTTTAGCT
ACGCTATAGGAAAAACCATATAAGGCTTTTATACTGGCCAATGACTTCACAGGCCTGAATTTTAGAAAGCCCCCTTC
TGCAGCTTGGCCTATAGATTCGAAGGAAACAGAACTAACACAAGAAAGCTAGTTAGGAGCTAGTTAAAAATCATCCT
GACTTGCCAAGGAAAGGTGCTGAAGACCTGGGTCACAGAGCAAATGCAAAACACTAGGACTTTGTCCCTAGTTCACC
ATTAAATCAACTTATTTTCTCTTACCCCCTCATATTCACGTTTACTCCTTACTTTGTAGTGGTTGGACAAAAATCAA
ATAAATCTGAGAATTCTAAAATGCACACCCTTGTTTATTTTCTAACTCAAATATGCCACTGTTGTCTGTGCTCTGTC
AAGATTTCAACACATCTTTTTCTCCTGTTTGCTTTTCCTTTTGGCATATAGTGAGTGTGTGTATACACACACACACA
CACACATTTTTTTTGACTCCTTCCAATGCCCTTCTGCTCTCCGCAGATACACTTCTGCATTCTGAATAAAACCGAAT
ACATATATATATATATATATATATATATATATATATATATATATATGCACACATATTTTGAAAACCTTATTTGAAAA
GAAAGCTTTCGGAGGAAACGTTATTTAGCCACTTAATCGAGTCTTTTACTGAGGGACTTTTTGTCGTCCCCTAACTT
CCTGTCAGCAGTCCACAGGCAGCAGGAATAATGTGGGAGAAGATCAACAGGCTTATTTCAGGAGGTCAGGGGCCAGT
GCCACCACCTGCAGGTGGAGACATCAGAAGCAGGAAGCAGCCCACCAGCTGCAGGGAGAACTCCCCACAGAGCCTAA
CCAAGATGAAGGGACTTGTAAATTTCAACCCTCCCTTTTGGCTTTTGTGCTAAAAATGTGAATATTGAGGTCTGCCC
TGATTAAGAACTAGATACATTCCTCTTTGTGACTGCCACACTTCCTTAGCGTATTCATTTTTTGTCTTTCGATCTCA
AGTTATTATTTTCAAATGCATTGCACGTATCTACCATGGATACCATTGCAATTGGAAGGAGCAAACGTTTTGTATGT
TTACTTGACAAAGAGAAGTGACTGCCCAAGCCACACAGAGTTCTGCACAAATCAGTAACTTCTAACGAACGTTTGCA
CTTCCGGGCTTGTTCTCTACCTATTTCAGTCGATGCATTTGTATTATTTACTTCAAACTCCAATACTAATAATGCCT
CAATCAGGTTGCAATTGGGATTTGAGCAGCCAGAATTTCAGAAATTTGGTTTGGTCCATATCTGTGACAGGTCAGTA
AATCAGAGAAGCAAGGGTTTGGTTGCTATTATAATACATTGCTTACCTATCAATTTAGTTATCAGCCAAGGTGGTTG
TTATCATCCAAAGTGGCTCATTAACCACCTTGGAGACTCAGTATACAATTGCAAGTAACCCTGGAAGTTGTAAATAA
TCCCAACTGAATTTGTATGAGTTTGGTAAGGTTAAGTGGAAACCAGCTGCTTAGGGCCTTGATTATAAATGAAGTTA
GGAGTGGAAGAAGTAACAAAACCCCAGGCAAATTCATTAAACATTTTTTCCCTTCAACTTTATGCTCACGAATGTGT
TGAGACTCTTCTGAATCCATAAAACACCTTTCAGCATCATCTGGGCAGCTTGATAAAGGCTGTAGACTGCCTGCCCT
TCATCCCAAACCTACTGAATCAGTGTCTGTGTGTGAAGACCTAGATTCTGACTGGTAGTTATACCAAAGTCTTAGAA
GCAACTCTAGGCCAGTAGTACTCACGTCAGAATCAGCTGGAGGGTTTGCTATACCACAGATTGCTAGGCTAGCCTTC
AAAGTTGCTGGTCCAGTAACTTTGGTGCAGGTCCAGAATTTGCATTTCTAGCAAGTTACCAGGTGATGCTGATGCTG
CTGGCCTTGATCATGCTGTGAAAACCACTGCTTTAGCTAGGCTATAAGAAACCATATAACATGGACAAGGCAAATGA
AAAGGTTGGAATTCTTCTGAATCCCAACACATTTGTGAGCATAAAGTCGAAGGGAAAATGATTCTTCTGAATCCAGA
CACATTTGTTTAAGGATAAACTGTTTTTTCCTTCTGAAAATTTAATGTCTGATTCTCGTTCATTCATTCATCAAAAG
TTATCAACTATCAACTATAGGTAGGAACTGTGCAATATGCTGGTGATAAAGAGATGAAAGACACAGCCCCTCCCTTC
AACCAGCTCCTAGTTGAGGTGGCAAGTCAGCTGTATAATCAAGTAATTGCAAGACTGTGCACTGAAAAGGGTGACCA
CAGGGTGTGATGGCCACCCAGGGCTGTGGAATCAGTCCCAAAATGAAGAATGAAAGCAGGGAAGGGTAATTCAGAAA
GAAGAAACAGTTCGCATAAAGACCCATAGATAAACATCAATCAGATGTGGTTAAGACAAAAGTAAGTTTCTGGAGGC
TGAGGACCTTCTCAGCTATATGTTTGCAGTGCTTGGTATAGGGCTTTATGCATCTACATGGAAGACAGAAAGGGCCA
CATCACAGTGGACAAGGCAAATGAGAAGGAGGCAGTATCAGAAGATGAGGGTACACCGGAGATCCTAGTTATATATG
GGCATTGTGTTCATCTCAGGAGTTACTGAGTAATGGGACCTTGACTCAAATGAATCTCAAGTCTGTTTTTGCCTAAT
CTTGGTTTTAGGACTAGGATTAGCATACAACCGCACTAGGAGCCTAGTTATACGAAAGGCTGCATTGCGGACCTGAT
ACAGTTCAATATACATACTGTCACCTTGCAAATAGGGTTACGTTAGTTCTCAAGACTGCCAATCCTCTGTGCTCTAA
TCCTTTTGGCTTTTTTTTTTTTTTTTTTAACTGTCTCACTCTGTCATCCAGGTGAAGTGCCCTGGGATGATCTAAGC
TCACTGAAACCTCCGCGTCCCAGGTTCAGGTGATTCTCATGCCACAGCCTCCCAAGTAGCTGGGATTACAGGTGCTC
TGGCGCCACCAGGCCCTGCTAAGTTTTGCATTTTTAGTAGAGACAGGGTTTCACCATGTTGCCCAGACTGATCTCAA
ACGCCTGACCTCAAGTGATCTGCCCGCTTTCCTTTGGCTTTTAACACTATAGAGCAAGGGTCCCCAGCCCTGGGGCC
ACAGACCAGTACAGGTCAGTGACCTGTTAGGAACCGGGGCCCCACATCAGGAGGTGAGCTGCAGGGCCGCCAGCATT
ACCACTTGAGCTCCACCTTCTATCAGCTCAGCAGCGGTATTAGATTCTCATAGGATCACGAACCCTATTGTGAACTG
TCCACACGAGGGATCTAGGTTGTGTGCTCCTTATGAGAATCTAATGCCTGAAGATCTGAGGTGCAACAGTTTTATCC
CCAAACCATCGCCTCCCACGCACCTCTCCCCACAACCCCACCCGCCCCTGATCCATGGAAAAATTGTCTTCCTCTAA
ACCAGTCTCTGGTGCCGAAAAGGTTGGGGATTGCTGCTATAGGGCGATGGTTTTCACATTTGATCCTGCATCAAAAT
TTCCAGGTGACTCATTAAAATACTGATTGCTGTGCCCCACTCGTAGGAGTTCTGATAAGGTAGCTGTGGGGTGAGAC
CTGAGAATTTACTTTTCTAATAAGTTCCCAGGTCATGCTGATATTGCTTTGATAACCAAAGCAATATCAGCTTTGGT
TATCAATATATAACCAAAGCCACATAGAGGGGGAGAAGTTCCTTGGGTTTAGCCCAGTGTTTACTGCGACCACCAAA
ATTGCTGGAGCTTAACCATGGCTCAGAGAGTTATGTTCTGTTCACTCTGTAGGCTGCTATTCCCTGTCACCTTTTGA
ACTATGATGGAGGGGAAGAGCTGCCAGCTCAGGAGATTTCACTTTTTTCTCTGCATAATTGAAAATCCAGAAACACA
GGGTTTTGGGAAAGCTATAGAACAGATCATCAGTGATCAGTGTTTAATAAAGTAAAGCAATAAACTTTACTGTGTAA
AATAGGATACTTTATTATATAAATTTTGTCCCCTTCCCCCACCTCACAGGCCAATAAAATAATATACTTCTTGTCCC
TGGGTGTAATGTTATTGGAAACCTTTGAATGTAGGAGAGGCATGGGCTTGTAAGTTGCAGAAAACTGCTAGCCTAGG
ATTGAGAATTTCATGGATAATCCAAAAATAGATGATTTTACAGTTATAAGCCTTACGTGAACTTGAGGTAAGAAAAC
ACAATGCCTTTATAGTCTTCTCAGTTGCTCCACATGCCCTCTGAGATTCTGTTCTGCCCAGCCTCTCTGGTTGTCAC
ATCTCTGGGCATTAACAGAAAGTTCACATACTCTTTGTCTCTGATGATAATCCTTCTAGGTCCATATAGAAGATCCC
TATCCAAACCATCCCCCAAACAAACCTATTGGTTAAATATTTTCTCCACCGAAGGCACTTTCTTAGATTCTAAGTGC
CCTGTAGGCAGGCTTCCTCTCTGATTTGGGAGAGTACAAATTGCGACAAGGTTAAATCATAGCCTGGGAATTTGACC
TAAAATTCACTCTTCTCCCATATGCATTCATGAACCTTCTGCTGGTTTTTAAAAGAAGCTACTTAATGTCAGCTCGA
AGAGGTTGGAAGGGGTTAAAAACATGAGCATGGCAGTAAGAAGATTTATGAAGGATCTGAGAAGATTATGACTTGAT
CAGATGGTATTTTGTCAGCTAGCCACATTTGTGAAGACTTGAAAACTAGGGAGGCTTGTCCTTCTAAGAGGGGGCAC
TGCTGGGACCTGGATTCTGTGGAACCGTATTAGTAGAATAAACAATAACCTTTGCTTGTATCAAATGAACTTCTATT
CTCATGTGTCTTTTGACATATTTTTATTAATCATATCACTGGGACCTCCTTGCTGAAAGATATCTCCGTTCCCCATT
CTGATGACTCCCAACTAGGAGTGAGATCAAATGAAGATGGCATGGACCATTTCTCCATGTGACAGCTCTCTGTGGTT
GCCTTTTAACACTTCTAATGCCCTTTCTCTTAAGAATTCCCATTTGTCGTCTGGCACTGGTGCTGTGATCAATAAAA
ATGTAATGGAGTGAGGCTTAGAAACATGAGGAAATTTACTCAAGCTATCCATTTATTGATGTGTCCATTTGTGTTGT
CAGGGAAGAAAAACTTTTTCACTCCCCTCTTAGGTTCATTACTTGGGGGGCTGCAAATTAAACTGACGACAGATAGA
TTGGCAATAGAAAAGACAAAGTTTATTCAGAGAAGTATGTGGGAGCTCACAGAAAACATAGCTCAATGAAGTTAGAA
TTTGGGGCTTATGTACTATTTTAACAAGGGTTTTGAAAAGAAGAGTGTTAGAATTTCAAGCCACAAAGTTGGTGGGA
AATATGAAAGAAACTAATGAAAGGTAATGTTTGTTTTAGTAAAGTCTGTTTATGTAATTTTCTTTTCCCAGCGACAA
CTTCTCATCTCTGGTGACAGGAGTCACTCTTTACCCCTGGTGCAAGAAACTTTCCTTAAAGGAGGATTTAAAACAGT
TGAATTATTTCAGAAATCTTTGCTTTTAGGCAGATAGGGGGAGTACAGAAAAAGCCCCTTCCCGTATCTGTTGATCC
TCAAATGGCTTTAGCTCAAAACAATTTTTACATCACGATGGCATAATGTAGATCTCTTCAATGTGTTCATTTATTCC
ACAGATATTTGTGAAGTACATGATATATGCCAGGTACTTGGGATACAAGAATACATAAGTATGTCCCTAGTCTCGTA
GAACTTACACTCTAGTAGTGAGCTAGAGAATAAATGATATTATTTATTATATGCATACACATATGATTTCAGATAGT
GATCCATATTGGAAATAAAGCTGGTTAAGGGAATAGAAAATGATATTGAAGGTGGACTTGTTTAGATTGGGTGGATT
GGCATGGCTTCTCTAGGGGGCAGTATTTGAGCAGATATGAGAGCAGATATTCTCCAATTTGGGCAAAAACATTCCAG
GCAGAGGAAACAAGGGCAAGGGCACTGAGTTCAAAAGAGACTTGACCTAGCCAACAAATAGCAAGGATTCCAGTGTA
AGAGAAGGTGGGGAAGGAAGGAGGTGCAAGTATAGGCAAGGGCAAGATCACACGGGATCTTGCAGGCCGTGATAAAA
GAATTTAACTCTTTCATAATTTTGACAGGACATCATTGAAGAATTTAGAAAAATAGAGTGGAGATACCTGATCTGCT
TTCTTCAAAGAGTTCATTCATCATTGCTGAGTAGAGGTTAGACTGAAATGGAAGCAATAGTGAATACAGGGAGATAG
CACAGGAAGCCACGTTACTAGTCCACATCAGAGGTGGTTCAGACTAGGGTGGAGTGGTGGGGTCAGTTAGAGAGCTG
GTATTTAGGATACATTTTAAAGACAAAGCTGACAGGATTTGCTGTGATGAATTAGATGTAAAGTATGAGAATAATTG
AGAATTATTTCTAAGTTCTTTGCTGGGGAAAAGTGGAGGAGGAAAAAGTTAGGGTACAAGGTGTGATGAAATCAAGA
GTCTCTCTTATTATCAGAGTCTCATTAGATATCCAAGTGGAAATGCTGGAAAGAAAGTTGGGTAGATCAGTCTGAAG
CTGAAGACAGATACTGTGACTGGAATAATAACGTAAGAGTTGGCCGGACACAGTGGCTCACTCCTATAATCCCAGCA
CTTTGGGAGGCCAGGATAGGAGAATTACTTGAGCCCAGGAGTCCAAGACCAGCCTGGGTAACACAGCGAGACCTCGC
CTCTACACACACACACGCGCGCAAAAATTAATCGGGTGTGGTGGCACATGCCTGTAGTCCCAGATACTCAGGAGGCC
GAGGCTGAAGGATCACTTGAGCCTGGGAAGTCAAGGCTGCAGTGAGCCGTGATCACACCGCTGCACTCCAGCCTGGG
CAACAGAGTGAGACCCTGTCTCAAAATAAATAAATAAATAATGTGGCAGTCATAGGCCCTTAGATGGTTTTTAAAGA
CATGGGACTGGATGAAGTCTTCTAGGAGGAGAGTTTGGGAAAAGAGCCCGAGAATTGACTGCACCTTTCAAAACAGG
AGGAAGAAAAAAAATACTCAAAGGAGACAAAAGCAACTTCTGTGATTTATAGAGAAAACCAGGCAAGTGGGATGAAG
AAAGTCCTTCATGATAGAATCAAAAACAGTGTCAAATGTTGAAAATACAATTAGACAAACACAAAAGAATAGACCAT
TGGGTTTTGCAATATGGAGCTCATACTTGACCTTGATAAAAGACATTTTCACTGGAAGCATGCATCAAAAAACTATT
TGTGGTAGGTTAAAATGTAGTAGGAGGTGAGGATATACAGACAGTGGCTTTCACTGTGCAGATACTGCTGCTCATGC
ACTAATTAAAAGACATTTGTTGAGTATCTACTATGTTGTATCCATTGCTAAATAGTAACAGCTGGGTTTAGTCAGGT
AGAACAGCATCAAAATCATTATAGTATCCCAAGATAGGTACAGTAAAATCTGTGAAGGAATCAGAGTAGTCTCTTCT
CCAACAGAGCGTAAGACCCAGCTTCACGGAGAAGGTGGTAGATTAGCTCATCTGGGAGGCTGAGTAGAAGCTTGTCA
TTATAGAGGGAGAACATCAGAAGTGTGGACAACAGCTTGAATAACCTTGAAAGGACAAAAGAGGACGGTCTGCCCTG
GAAATATTAAGAAGTCTCACATGATTAGACACAAGATATTAGGGGAAAGGCATAAGGTGAATTGAGTCAATGAGGTC
AAAGAGAAGCTAGCTGGAGGAACAGGCGATCATAAAATGAGTAAAAGTATATATTCAAAGATTCTTTTTAGAAGGGC
TACACAGGATGGATAAGGGGAGAGAGAGAGTTGAGGCACAGAGACAAATTGGAAAGGTGCAATCATAACCAGAGACA
TGAAAAACCCATAGAAATCTGATGTAGATTATGTGGTCCCCAAGGTTGAACAATTAAGTACGCTTTCAGTTGTTATG
CCCATGATATTAACATATTTTATAACTGCAATAAGTGCTGAAGCTAAAGATAAATACAAACAATGTAATTCTTATTC
TGTGAGAAAATGTTGTAGCTGGAAGTTAAACATGTTTCTTAGCTAAAGAAAAATATTGTGTGATCTGGATTACTTAA
TGTTATAATTTAGCAACAAAATGTTGACATTGAGCCTTGCATAATCAAAAAAGTAGTCTATTCAATAACCACATTCT
CAGAAAAAAAACAAGAAAATATTAGAAACAATGATAAATTATCGTAGTAATTTAATTCAGTATTCTATTGTTTTATT
TGGATTTAGGAAAGGCAGAAATGTTGAAATATTAATATATATCCCTGTAATAATATAATTTGTGTCTGAGAGGTAGG
AATGAGGGCATGAGGTCAAAGTTTGATAATGAACTTCAAAGCTATAACTATGATCAGGAAATTAAAATTGGACAATA
AATTCCTAGAATCGTCAGGAGTTGCTTGTGAAATCGAGAAAGGAAAGGATATACACAAAAATAAAGAACAGCCAATG
CTCTCAAAGGAGTCTAACTTTTATAATAGTCTTCTGTGTTAGAGCTGAACTCTTCTGGTTTAGAAGGACACTCTGTT
GCCTGGAAATAGGGCATGGAAAAAGTCATCAGAGTCATGTCATCTTTCATTCTTCCCATGAACGAAATCGAGGCCCT
GAAAAGTCACCTGTGTTTGCTGTATTTTATTGCAACTAAGATGTGCATTTTTAAATTGATACATAATAATTGTACAT
ATTTGTGGGATACATGTGATATTTTGATGCATGCATACCATGTGTAATTATCAAATAAGGATATTTCTGTATCCGTC
ACCTCAAACATTTACCATTGCTTTGTGTTGGGAACATTTCACGTATTTTATTATAGCTATTTTGAAATACAAAATAG
ATTGTCATTAACTATAGTCACCCTACTGGATGCACCTTGTTTTTAATATTTCTGAAAACAGATACGTCTCATAGGTG
ATGGTGTCACAGCTGTGCATTAGTTATTATTGCCTGTGCAGGTGCAAACGTAACTATTCATATTGTTGTCAATTAAT
TAAATAGTTACATTTATTTATATGCGTTTATTATACTAATAAACACAATATTGAGATAGTTGAGCTCTAGTTTTGAC
TCTGCTGTTAACTAGCTGCGTTACTTTAATTTACTTAACTAATTTGGCTTTCAAATTCCTGATAAGTAAAATTACAA
CATGAGTTTCTCCTGCTATAATAGCCTGAGAAATCGGTGAAACACATGAATTCAGATGTTGATGCTATTTAATAGCG
GGATTCCAGATATCTACTTGCCATTATGGGAGGGAGAGAGGAGGTGGACTGGAGGCTGTGATTTCCCTAGGAGGTTG
TTAAAATTGGCCAGGTGAGGAAAGCTGAGACAGACCATAAATATGAAGCATGATACCTAGCCCTCAGTGTTGAAAGA
AAATCAAATCTCATCTTTGTGGTCTAAATATCAGTATGATACAATCCTCTGTGTAGACATATCCTCTGCCCTATTGT
TTTCTTTCTAAAAGCTAAAGCCCAGGTGTGATCACATCCCTCCGTTATTTACAAATTTCTGATGATGATGATTCTTC
TAATATCTACATTCCTTACCATTACCATGATGTCCAAAACCTATTATAATCTATTCGTCTCCAAGTGCCATGTTGTG
GTCACCCTATGCACCCTCTAAACCCACCATATGACCTTCCCGCTGCTACTTGAATACAGTTGGCCCTCTACCTCGTT
GTGTCTTTGCATTGCCTATTTAATTGCCTTTCCATTCTCTAAATCACTCTTTCGCTGGACCAGCAACATCAGCACCA
TCTGGGAATTCATTAGAAATATAGATCCTCAGGCCTCATCTCAGACCTGCTTGATCAGAAACATTGGAGAGTGGAGA
TGAGCAGCCTGTATTTTTATCAGCCCTCTAGGTAATTTGATGCACACTAAAGTTTGAGAACCACTGGTCTAGAGCAT
TCTTCTTTAACTCTCTTCTAAAAATTATTAGAATGAATTCGAGGGACGGGATCTCCTTGAAAGCCAAGAACATTTCT
TTGTCATCTTTCTGACTTCAGGGCGTAGTACACTTTTTGGCCCATAATTAAAGCTCGATAAATGCATTCTATGCCAA
TAAATCAGCTAATCAAATATATTATTCATGCCCTTGAGGTATCTGAAATTTGTTTGCAGAATGTAATATATAACTAT
AGAGTAACAAGAGAATAATTTATTGCCATAGATAATAAAACAATATCCTCTGTATAATAAATCCTAGCCTCTGCTCA
ATGGGCAAAAACGGGACTGGGGTTTCAGATTTTAAAAAGATTATTGGTAATTAAATCACCTGGAGAAGCACTTGCTG
CAGAGATGGGACTTGAAGCATCATAATAAACTGTTGTTTATTATGATTCGGTCAGAGCTGATGGAATCACAGGGATT
GTGTGAGGTATGGAAAGTGGTTGACATTGAATTCCAGGCTGCACAGTTGGGACTTGATATGATAACCAAAAAGAAAG
AATGTCTGGGGTGGTAGCAAGCTCTAAATTTAGACAATCTAGGCTTATCCTAAGGAGAATATAGATACAGATAACTG
AAGTTTGATTAAAGGGAACCTGGTGTATCACAAATAGTAAAAAGCTGTAGTTAGTCTATGCAGCTATCAGCTAGCCA
CATAATACTTTTGGGCAAATACATTATAAACCAAAAGAATGACATGGCTTATCTCTGTAACAAAGTGGCTCATTGTT
CTTTATTCTACTGTTATCCTTAAGAAAAAAATTTTAGTAAATTTGTTATGCTATACTCAACTTCAAGAAGGGATAGC
GCTTATAAAAAAATTGTTTAAAGAAACAGGCCTATTTCTCTTTGGGAGAAGCCACGGAGAAACGAAAAGAATGGAAC
GTGTGTTTCTGCCCAGATGGCAATAAAATGTAGGGTAAATTTCTGTCTTTTAAAACTGTATTTTTTCCATCCCTCTG
TATATACACATATCCTAGGACTGTTATAAAATGCTGCATGCGTATGTGAAAATGGAACCTTATTGGGCTGTTTGATG
GACCTTTAAAATATATTTGTTGGTTTGGGGTACATACTAGCTATGCAATATAATCCGCATTATTTCTTATGTAAACA
ATGGATAAACTGTTTCACAGTCCAGACATTTATTTGGTCACTGTTTGTAGAATGTCTATTTTATTTACTTCTGAATT
TGTATTCCAGAGATCTGCCTTCAATGTTGGATACTTCCACTGTAATATTCTAGGAGATGCTCACTTTCTTTTTCAGC
ATCTGACACAGTACCATCTGCCTCCTCTTTTCTTGCCACAAGTAATAACAATTTTATAAAGGAGGATCACATTACAG
AATTATAGGTGGTAAACTTTCTACCACCAGATTTACCCAAGAACCTGAAACACATTTTTTCAAAAGGAAATAGAATG
TCCTTCTTGTGACTACATCGGAATTTTGCTTGCAGCATTATGCTTTTTTTTTCCCCCTAGTGTAGCTAGCCATGTGG
AACTGAAGCCATTAGCCAGCTCCTCATCCTATAAATGCTATTACCTGGGAAAAGAGGCAGAAAATATACTCTCTTCT
CCAGTTAGAGTCTAAAGGAAGAGAACAATATGGGTAGTTGTGTTTACCACAAATTGATAGAACTCCTTTATTTTAAA
TGCTAAAACCAAATAACTTGTTTATATGACTTCAACATTGACTATCACACACTGTTGCATGATAACAGAGTGAAAAC
TACCTCTATTGGATTTAAGTGGGGAATCTATGTCTCATTCTCATTCTTTTTTTACTGTGGAAACTAGTTGATTCCAG
GATCAGCCTTAGCTCCAACTTGCCACACTTTGAGTTTTGGTTTTTCACTTGCATTGTCACAGGAAACTTCTATAGGA
TAAATCGAGGAAGATTTTACTCTGCAACGTGTTGCAGAATTAAACATTTAAAGTGGCAAAACCTTCGTGTGTAGGTT
GTCTCCCCAGAGAATGTAAAAATGAATTGAAGGCAGCACCTAATAGGTAAACGACAGCCAATCAAACAAGAACAAAT
GAAATTTGACTGGCAAAATCAAATTGAAAATGTATAACGCTGAATCTCAGAATATAGGAGGATGCATAGAAACTAAG
CTGTACTATTATAAAAGTCATAGCCATTGAAAAATAATGACTGGTTAATTTGGTTTTCTTTACCTCATGGATGTGAA
TGGTTAGATTTTGATGTTGGTGTTATTTGACGTGTGTTTGTCAAGAAGTTGCCTTAGTCGGCTCGCATTTAGGATAA
AAAAAATATTTTAAGAAATGTTTAAGAGATTATGTTGGAGACATTAGAAACAAAATAATTATGCAGAGGGCAGGACT
ATCAAAATATAATAGAAAAATTACACCGCTCTTTTATGATTTCCTCCTTTTTGGCATTTAACACAAAACTTTATGAT
TACACACACCACGCACTCCAGAAATGCTTAAAGGAAGATGAGAGGAAAATTCAATAGAAGTAGCAGGCATTTCTGTG
AGGACAGCAGAATGATCACTTCATCTCTGTATTTTTTTTTTTTCAAATTTCTGTATCTGTACAATGTCTTTTCCAGC
TCTAATATTCTGTGATTTGGTAATTTCCGCACTCAGATTTTCTTTAATGAATTTTGTATGATATTACCTATTTTTAT
ACCAGATATTACCTGGCTCTAATTTCTTTTTCACCCTAGGAAATAAAAGTATCGGGTGAATTTCCCATTTTCTTATG
TTATTGATACAGGTCTCTGTTGGATATCCCCACGATTAACTTTCCTGCAGCATGTTCGATGGTGGCTTAAAGAAGAA
ACCATGTATCAGAGCCCCTTGTCTATATAGACTTTTAGATAAAGAGAAATACATATCACAGAATTATTCTGGGCGCA
TAGAGTCTCTAAATGCAAAAAAAAAATTGTATTGTAGCTGTTGATTCTTCTCAGATAGATTGAGTGTAGAGAGAGAG
CATTCCAAAAACTGAGCAGAAGAAACACAGTCTGAATCAAATAACATGAAATTTTAGCTAACAAGTAAATAACACTT
TTTTCAGAATATGCAAATAATATTGGTTTATTATGAAAAATGTATAGGCTGATAGATGAGCATAGAGAAAAAATTAT
AAATATCTTCTTTAATATCACTTTCCCCAGCAAACCACTTTTAACATTTTGATACATTTTCATGTTCAAACATTTCC
TAATAGTCTTTTTTCCTGTTATATAAATATGAATTTTAAACATTCGTATGTTTATGAAAAGGCAATAAGATACTGCT
CTTTTATAACAGGCTTTCTGAACTTCACAACATGCAGTGTATTCTAACATGCTCCTTGTGTTCTTAACTAATAAAAA
ACCTCACGTTATTTAAAAAACCATCTTAAACATAATTATCCATTAAGAGAAGAGGTTGGGGTAGAGAGTTTCAGACT
ATCAATATCAAAGTTATATTTTCTGTAAGTATTTTAATTTTTAAGTGTAGCTATAGGTATATGATTATAAAACCAAT
AGCAGAGAAAAGATACCACCTTTGAATATAGTTTTCCTTGGTTCCATGAAAATGGCCTCCTTTCTTTTTGCCAGTCC
CTCAGTATCATTAACTCATTTTTCTGTAAATGCCATCATTGTATCACATGTCCTCAGGAAAAGGCACTTTTCTCTTT
TAAGCTAGTGTTTGTTCTTGTTCTAATTTTATGGCAATTTAACGAGTAACAATCCTGTTTCTATAAATACTGTTTCC
TAATTAATCTATTGCATTCTATCCATGAGAATTTAGATGACTTTCTTTGTAAGAGAAATCTCTGTAGCATGAGATTC
TTCTTTGCTCTTAAATTTCATTCTTTCACATTTTTAAATGACCTGATAGTATTTTGTTGTATTTGTGCTGATTTTTT
TTAACCAATCTTACCTTGTTGAACATGTAAGTTGTTTCTAATATTTGCAATGATCAAAATGTGGATCCAACTTCACT
AAAGCGTTAAGAATCTAAAACAAAACAAAGAACAAAAAGTTGGCTGTCATCTTGCTTGGACCACCCCGTGAGTTACT
ATTTTCTTGTTTCCGGTCACAGTTCATCCTAAATCATTTCAGTACACAAAATGTTTTTTAAAGTTTGGGACAGGGGG
TAGAGAATGTCAATTATTCCTCCAAGGCAGTCATATGAGCATTGAGTATCATGTGGAATAGTTGTTACTTGTAAAGT
TATGGGGCATCAAACCCAGTCAATATGTTTCTGGAATTGAAAAAGTCCCTGGACATTCTAATGATACTGTTGTTCAC
TTTGCACCTACTGTTACCACTACTTTGATCTGTCAACACTGCCCGTAATGGTTAATTTTGTGCATCAACTTGACTGG
GCTACAAGGTGCCCAGATATTTGGTCAAACATTATTCTGGGTGATTCTGTGCAAGTGTTATCAGATGAGATTAACAT
TTAAATTGGTAGACTGAGTAAAGTAGATTGCCCTTCCTAATGTGAGCAGACTTCATGTAATTAATTAAAGGCCTGAA
TAGAAGAAAAACACTGACCCTCCCCTGAGCAAAAGGGAATCGTTCTGCCCGACTGCCTTCAAACTGGGACATGGGCT
TTTTCCTGCCTTCAGACTTTAACCACAATATTAGCTGTTCTTGTATCTCAAGTCTGCTCTACTTCGATTGGAACTAC
ACTATCAGCTCTCTCGGGTCTCCAGCTTGCTTGTTCACCCTGTATACCTTGGGAGTTGTCAGTCTCCATAGTTGCCT
CCATAATTGCATGAGCCAATTTCTTACCACATACAAACACACACAGAGACACACACACACACACACACACACACACA
CACACATATAATTATATATGTGTGTGTATACATATTCTCTTATTCCTTTTGTTTCTCTAAGGAACCCTAATATACTC
CTTATTACTCTTTCTACTGCCTTAGAGATCTTCAAGGCCAAGAGCGTAATCCTCCATCCTGGCTCTTTTTCCTAATC
ATTAATGATCAACTCATAGCCATTTAGCTCAACTAAAAATAATTTGTTCATGAAGCTTTACACTCCCACATACTGAG
GAACGTGGTACCTAAGATCAAACAGTCACTGCCTCATCAAATGCATTCCTCTTCAACCCCATACAAATGTCCCCAGA
TGGAACTCACACCATAAAAATATTAGATCCCATTGACTTTTCTGCTTTCTCAAGGATCATTGCAGAGCTTGAAAAAG
ATGGCTCCTCCCTTTGCCTAAGCAGGTTAACTTGGTGTAAAAGTACATGTAAGATTTGGCACAAAGGAAAATAAATC
AGTTTTGCCTGGGTCCTAAGAAACATTTCCCTCTGCCTCATGGTAATTGTACCTGCCAGTTGATTGCATTACTCAAG
TGGAGACCATGAAGTGAAGTGGTAGAACAAGAAGAAATCCCTATAATTTTATTAAGTATGGTGAAAAATACAGATAT
GTAGAGAAATGACTGGGATTAGATGGAGCAAAACATAATTCGAGATCCTGATACAAATTGTACTTCCTGGCTCAAGG
GAGGGAGCAGAACATTCCCTGCTACATGGGAATAATAATAAATGCCTGATAAAAATGCAGATATATCATAGACTACA
GAAGCTGAAGTGGATTCTTATGGTCCCCTACTCAGACAGCCTCTCCTTCAGATGAAGAAACTGAAGCACAGAAAGCT
CATCCTAGTGTTTCATATTGAAAAACCCATTCAAGTCTATTTTAATAACCTGTTACCAAAAATGAGGGAAATAATTT
AACTTTAATGTTTCACTTTGCATTACCCTTTTCCTGACTAGACTTCTATCCTTTTCTTGAGTTGAGCTCATTAACTA
CTATGAAATTATGGTTATGGGTAGAGGTTAATTTTATACCTGTCCATCTTCTGGCATCTTATTTACACTAAAAATCA
TTTTTAAATGGCTTCATTTTAAAAAATATTATTTCAGTTGACATTTTAAAAGACACATCATTTATGTACTACAGAAT
ATGCATTTTATACTCTCCTTTATTAATTTTATTATTTTCCAGGTAGACCAATCAAATGAATCAGAAATTCTTGGTTA
GATCTATTAGACAGCATAAGTATGTTTTTCATCATTAAATTAAGATGAAAACACAATTTTACTTTAAAGTGTTTGAC
GTTTCCAGCCTTTATAAAGTCAACACTTAATCACATCTGAAATTTGCAGGAAAAAATTTTGAAAGCCTTCAATTATT
AACATTATTTCGGGAGAAAAAGCCACTTTGCCGCAGAACTTTCACTTTTCTCTCGTGAATTAAGTCTGATACAAATT
ATTCATTATGGTGAAGTTTAAACATAATAGAGTCTAGCTACTTCCACAAAAATACTATTCAATGAGTTTCTACATTG
ACATCTAACTGACCTTGTAATTAATGTTGTACACGATCCTTTTATTATATGCTGGATTATCAAATATGACTTATTAG
CAGTATAAAGACACAAAGTTCTGAAATGTAATTTATAGCCATGAAAAGGAACTGAGCTTTGTGTGACAGTTAAATTT
GAAGAGATCAGGTGATTATTATGAAGCATGAATAATAATGCATATTAAACTCACGTTTTTGTTTAAATCATTAATAT
GATTGTTTTAGAAGAAAGTCTACCTCTATCATATGGGCAATAAAATGTGTATAAGAGCAAACATTTGTGTATGTGAA
ATAACTCAAATTAAAACCAGTTTTCCACATTAATTCTTACAGTTTTTAAAATTTAAATCATTTAATGTATCACACAT
AGCTTTATTCATTTTAAGCTATAAATGTTACAATTTCTGTTTAAGCTGTTAATATAAGCTTTGTAAGAGCAATTCTG
TATAAATATAGAATTGTCATTATTCACTAATAGCTACCATTTATTTAGTGCTTGTTGAGTGCAAAAGTACTGCACTG
AGATCTTTGCATATGTTCTCTTAATGTTACAATTCTTACCTGAGGCATTTCTGTTTCTGCTGGAATATGGTCTCTCT
GAATTGAACAAGGGAGGCATTTTTGGTTGTTATGATGAAAGGTGGACACTGCTGGCACTAACGTGTGTTGGTAAGCG
ACTAGACTCTTCATGATGCGTAAACAGTGTTTCCTCATACCCCTGCACATTCAAATAGAGGAAAACCTTGTTTATAG
TTAATTTCCCCTAGAATGTAAATCCATTTAACATATAAACACAAAGCGTGTTTTGTGTGGATGTTTTTTACTGGAGC
AGGGAGACAGGAGAGGAAATGCAGTTTTGATAGTTGCTGAATTTTTCAAGAATGCAGCAATTATAGAACAATTTCTA
GAAGTTTCCTAGGAGCTCTTTTCCATAGCAGAAAACTAGGACTTAATAGCCTTGCGACTCATGGTACTTGAGTGTTC
CATACAACTCACCTATATTCAGGGGACATTTGAAAAATTCTACATTAAAGGGGATTCTTAACATAGGCGCAAGTGTC
TGGCATCTTCAATAGGTCTTCTGGTGTGGCCATGAAAACATTCACACGTTTCAAAGTATTTTAAAATAAAATAAAAC
ATATATTGTTGTGTTATGAATTATTTTCTTTCTTTTTTATATGATGGTTAGATCACTGTGCAGACAAGTTTATGAGA
TCTATTCATTTCATTTCAGGGTGGTAAATGAGGGTGTTACTAAATGTTGGTTCTAAAAAGGGAGACATTGGGTATTA
CAGAATTCAGAACAGCTCTAAGCCCTGTGCACATTTAGCATTAGAGGACACAGGCAAATCTGGCCTCCAGTCCTGGC
AGCTTCTTCACTATGTATATGATGTTGGGTGGGTTGCTTTACCTCTCTAGTTTTTACTTTTATTTCTAAGCTAGGGC
TATTCATAGTTCTTTATCATGTGGTTACTGTGAAGTAGCAAAGCACCTGACATAATTAGAGCAGATAAAATGCTCAA
CAAATATTGCTTATCAGAAGGATTATGTATTACCTCCCGAAATACATCAAAAATATATTTTCCAATTCAAAGAATAT
GTAGTACAAAAATCATGCCTAAATTAACAGAGTTGCAGTAGCCCAAGGAGAGAAGATAATCATTATTGATTTCTTCT
TCCTTTTTGCTAAGCAGTTCTCTGTCTCTGCCTCCTCAGTTGTTGTCCATCCCACTCCCCCACTCCCAAGCCCTGAA
CTCTGAGGGGTTTGCTGCCGTGGCCGGTTCTGTAGTCATTGCTGTCCAATGATGAAAACACAAAATACTGCAACAGA
ACACTATGCCTGTCAGCTTAGCTCCCTTCTTTCTGCTAAATGACACTCAATCCTATTCTTTTGTTCTAAAGGATATC
CTAAATGAATAGCCACTGGGGGGAAAAAAGGTTATATAAGATTGTGCACTGTGTGAAACTGATGCAACCAGATCAAT
GATGTGAATTTCTCTTAACTATTTACTGGGATCTAGAAACAGGTCTCTCAACTTAGCAGTGTTTACGAATATAATAG
GCCTTCCTTATACATACATCTGAAGCCAATCTGAGTCAGGAAGAGTCGTGGTCTGATAAATATTTTGAAAACTTGCA
TTTGTTCTATTAAAGCAAACTGTTTATTAATAGTGTGCCTTATTTTTTAAAGCAAAACATTTATAAACAGTAGTCAT
TACAGGCACTTCAGTGTACGGAGTGATCAATTGTTAGACCTTTAGGAATCGATTGTTTCGTGGAGCTTCGGCTTATA
ATTGAAATGTCATCAGAAGGAGTGTAAGACATAGCTTCAGGAGAGGCCATTTATGCGCTTTTGTTTTCAGCTAAGTT
ATAGAGTCATCATGTGAAGAAAGATTCTTCTCTTAGTAAAAATCCTTTAATGGTTGGAATAACACTTGATATTTAAT
ATTTCTTTCTACTTTATATCCACATTTATTCAAGTGCTAACGCGTGTGGGGCAGCAATGAAGCACTTTATTCCAACA
TTATAGTTCTCATATCTGCGTATGATTATTTTTCATTTATCGTTAGCATATATATAATGATGACTTTTAAAGTACAC
TGTATTATATTCACTGGAATAATGATTAGCTATTAATAATTTGAACACTATCCAGGAAATTACTGAACATGTCCTAC
AAGATAAACCTCGTATGATATTGTCTCCAAATAACAGTGCTAACCAAGAAGAGTGCTACCAAGTTCAAAAGTAATCA
CAGGGAGTAACCTAAATGCAGCTCCGTTGGGTTAAAAATAGTTTCTCTAAATTATATGTTCCCTAAGTTTGAGATCG
ATTTCTACAAGGGGATAAAATGTTTTTATAAATTCTCAGTGATAAGTCATGTGATTAAGAACCCCCAACTTTTTTTC
CAAAGACATTTGCATCTCTGATCAAAATAACAAGATCCAGTCTTAGTTATAAATTGGGGAATTTTCATCAAAATAAG
GAGCTACTCGTTGCATAAGAAGACTAGTACAACTTAAAGCCAATTTAATTTCAATGAATGCATGATCAGCTCCATTG
CCAATTGAGTGTTTTTCTTATTCATCAGAAGATGGGTTCATCATCGTGTTTCATATCAACTGTTCTCAAACCATATT
GCCCATTTAAATAAATATAGATTTGTCTCGAAATTCTAAATTCATGTCATATTTCATAAATAGCCTATGGTCCTATT
TATTACTTTAAAATATTATAGATATAATATTTTTATTCTAAAGTAACTGTGTTATACAACCAAATTATTCATTTAAA
TATGTGACTTTTTAAATAAGTAAATGACTTATTTAAGTAAAGTCATTAAAATTTTCCAGTCTGTCCTTCATCCACCT
GATCTTTGAATGAGTTAGGAACAATACAGGAAACTAATACAAACTTAATTTTGATTACAAAAGATGAAATCATTCTG
TTATTTATTCAACACACTATGTGTCAATAAAATCTTATACTGTGAAAGAATTCGTCTAAGTCCATTTGCTGTTGCTT
GTAACAGAATACCTGAAAATGGGTAATTTACAAAGAAAAGGAGTTTACTTCTTACAGTTACGGAGGCTGAGAAGTCC
AAGGTTGAGGGGCCACATCTGGTCAGAGCCTTCTCCCATCCAAGTACTAACCAGGTCGAACCTCACTTAGCTTCCAA
GATCAGATAAGAGTGGGCGCGTTTAGGCTGGTGTGGCTGTAGACTTGTTAGAGCCTTTTTGCTCATGGGGACACAGC
AGAGCCCTGAGGCAGTGCAGGACATTACATGGCAAGAAGGCTGAGTATTCTAATGTGTTCATGTCTCTCTTCCTGTT
CTTATAAAATCATGAATCCTACTCCCATGATAACCCATTAACCTATTAATTTATGAATGGATGAATCCATTCATAAG
GGCAGAGCCCTCATGATGCAATCACCTCTTAAAGGCACAATCTCCCGGTGCTGCCACGTTGGGGATTAAGTTTCCAA
CACATGAAATTTGGGGGACACATTTAAACTATAGCAAAATTGTAATAAAATGTTATATAGAAGCAATGTTCTTACTG
ATTATAATTGTTATATTGGTAAAGTGTTAAGTCCTCTAACCAAGGGATATATTTCAGCTTATTATAATAGTTTTAAA
TTTACAATTCAATATGAATAACATCTGGTAAAAGTTCTTTTCAAGAAATGGGAAAATTAGAAATGTTTAGAAGAAAA
TAATTCAATAAATATTAAGTTCAAACTGGATTCATAGTTTATGTGAAATTCTGGGAACCAATTGCAAGGGGAGAAAA
TAGTTACAATAGCAATGGTGAGGATGAGAATAAGAGCAGGTATCAACGTTAATTGAGGGTGTGTTATAGTTCTAATC
GTGCTATGCCCACTACATGACTTTTCCCTGTGTGAGGTTTCCGAGCTTCTTCGTAGTAATCCTAAATTGAGCTGGAG
AGAGGCTAGGGTAACTTACTCACGCTCATAGAGCCATAGAGTAGTAAAACCTGTATTTGAACTCTGGCCTGTCTGAC
ATCATTCTGTGGTCTTTTAAACCACCACTGCTTCTCCATATTAAAACTCCAAATCTAGGTGAAAAGAAGAAAACTCA
GAACATGTTCTGCAACAAAATATAACAAAATATAATGTATATAAACACTTATACATAATATCACTAATATCTTTACT
ATGAAAAGACTCTGATACGAACATTTTACATAATTCATGCAGAAGTGTTAATCACATTGTCTGTGATGAGCTGTGTA
TGTATCTGATAAAATTCTGGCAACCAGACATCAACTCGTAGGCATAGATCTGTAACACTAAATATTTGCCTCGAGAA
ACTTAAAGAAATAAAGACAAATGAATGAATAGGAACATGGAACTGAGTACAAGATAAAATCCTCCTAAAGCAATCGA
TGTACTTGCTGCTGCGTTATTGTTCTAAGCAAAAGAAGCATGGCGAAGGGAGATGTGAAGCTAAAAACAGAATGCTT
AGAAGGAGATGATAGCAGGAGGGAAGCAAAGATGGGACCAAGCTCCCAAAAGGCGGGCTTTGAACAAACAAAACAGA
AAGCTAAGCCTTTGACGGATGCACGGGATGCAAGAAACTTTAGTCAGGAAAGAGGAGGCGAAGAAAAACCCTCCAAA
GAAAAGGTGAACAATATTTTAATAGGCAAATTGACAGATAGCAAGAGATATATACCATGCTATGTTTTCTCATTGCA
GCTGAAGACAAACTGGGGTTATTTATGCTTTGAAAAAGCGTAAATCTAAAAAACAATTGTGGAGGAAGAAGCGATGA
AAACACGTGTTAATACAGAAAACATGGCTCCAAGGCTTTAAACTTCCTTGTGAGATAAATGCATTTACATTTTCCGT
AGTAGCTAATATATATATATATACATATATATATATATATCTGGGAAAATAATACACAGTGATTTTCTTTCTTTTTT
TCATCTACTTATGTGAGAAAAAAGTAGGCTATCTGAAAGCTTTTCAGTTAAATGAGGAAGAAAGTTAGGTGATCTTG
TAAATAATATATATGTTCAAGATAATGTAAGGCCCTTGTGTAGTTTTCAAAACTTATCTTTAATAGCAGTTTCTTCT
GGGGATGGGGTAGTTCAAAGTTGAAATGTTAGAAAGATGTTAACTTTTTTTCCTTTTTACTTCTCCCTTTCAGGATG
GAATTAACAAATTTGATTACAAATAGATCTCAGAGAGAGGCAAATGCATTGAATCCAGAAGTAACATAAAATTAGAT
CATGTTTAGTTATGCCCGAGGTCACATGGTGATAAAAATGAGGATAAACTGAAATTGTCTGTGAGCCAGATTAGTTT
ATTTTATGCCAGTCCTAGGAAAAAGACACATCATGGTAGGATACATCCTTTTTTTTTTTAATTATACTTTAAGTTTT
AGGGTACATGTGCACAGTGTGCAAGTTAGTTACATATGTATACCTGTGCCATGTTGGAGTGCTGCACCCATTAACTC
TTCATTTAACATTAGGTATATCTCCTAATGCTGTCCCTCCCCCCTCCCCCCACCCCACAACAGTTCCCAGGGTGTGA
TGTTCCCCTTCCTGTGTCCATGTGTTCTCATTGTTCCATTCCCACCTAAGAGTGAGAACATGCGCTGTTTGGTTTTT
TGTCCTTGCGATAGTTTACTGAGAATGATGTATTCCAGTTTCATCCATGTCCCTACAAAGGACATGAACTCATCATT
TTTTCTGGCTGCATAGTATTCCATGGTGTATATGTGCCACATTTTCTTAATCCAGTCTATCATTGTTGGACATTTGG
GTTGGTTCCAAGTCTTTGCTATTGTGAATAGAGCCGCAATAAACATATGTGTGCACGTGTCTTTATAGCAGCATGAT
TTATAGTCCTTTGGGTATATACCCAGTAATGGGATGGCTGGGTCAAATGGTATTTCTAGTTCTAGGCCCCTGAGGAA
TCGCCACACTGCCTTCCACAATGAACAGACACTTCTCAAAAGAAGACATTTATGCAGCCAAAAAACACATGAAAAAA
TGCTCACCATCACTGGCCATCAGAGACATGCAAATCAAAACCACAATGAGATACCATCTCACACCAGTTAGAATGGC
AATCATTAAAAAGTCAGGAAACAACAGGTGCTGGAGAGGATGGGGAGAAATAGGAACACTTTTACACTGTTGGTGGG
ATTGTAAACTAGTACATTCTTAACATCAATTTATTCCTAAAAGCAATGTTCATAGGGCACACTGTAGGCCATAGATT
TGCCTCACAAATTTAAAGGCCTAAGCCCTCAACATGCACAGCAGTATACTCAGAGACTATTTGTAAAGATGACGATT
CTGGAACTTTTTAATGACCCCAATCATTAGCAATGATTAAAATTAATATTCAACATTCTATATTTACCAAGGCAATA
AAGTAGACTAATCTATTTTAAAAGGGTTTTAAAATGAAGAGATGAAACAAACCAAATGATTTTGATTTAAACTTCAT
GAAAACATAAGTTGCATTAATCAGGTGATTTTGTTTTATGAGCATTCTGATTGAAGTGATCATATTTAGCCCCGGGA
GAATAAGAGAAGGTAAAGTATGGGTATGGCACTGAATTTACTGAGATGATTATATTGTTTGAGTTAAAGAACTTGTA
TTAAGAAACAAGTATGTGCCAAACATTGTGCTAGGAGCAAGCAATGCTAAAATTACATGGGTAGAAAGAGAGAATGA
AATATCTAGAATGAGTTAGAAACATCAGTGTTTTCCAATGTGGAGCCCTGACTTCACATGAAAATTCTCATTTTCAA
ACAAGGTAGTTTATGAAAACTGGACTATTAGCAAGACAGGGTGGGCATGCCATCAGTATAGTACCTGGTGTAAAACT
AGAAATTTTAATCATTTGTGCTTTCATTTTATAATCAGTAAAATCCAAGGTAGGACAAACTTTTACTTTTTCTGTAT
AATGGACTGATATTTGAATTATACCCAACTTTAATTTTTTGCCAGAAATTATGCTTTATTGTTTCTCTAAAATGGTA
CTATAGATCTTTATTTATTTCTATATATTTATATGATTTTTACATATATGTGCATTTACATGTATATACATCCATAA
ACTATATACATATATACACATAAATTACAAATATGTGTACCTACGTACATATATATGCATATATCACGCAAATACAG
GCACATTTTCAATACCCCTTTTTGATTTTTTTCCTTGAAGAGCATAGCATCTGAATTTATTATGGATTTATTTTTAA
TTTATGGTCATGTTCTTTGAGTGCTTTTGGTGTTTATCTGGTTGCCCCAAACTCGCTAGCATTGTAAAGAAGATGTG
CAAAGCCTGAATCTAGACTGACTTTCATATTGACTTTATTAGTCAAAAAAAGTAGATGAAAATGTAACAGTCCGTGT
TAAAAATGGGAATAAGACAGATGTTCAAGCCCTAGCTTCAGCAGTTTTTAGCTGAGATTTACTGGAAGAAAACATTT
TCTGAACTGTAAAACATGCAAAATGCCTACGTGACAGACTTCATTAACATTATTAAATGCTATGATATAGTAAAAGA
ATTTGTAAACTGTCAAGTGCTTTGTCAACATTAGGAATTTAGTTATTATAGGTATTTCCATATACATGTTGTATTTA
GAATTCCCTTTAATTTTATACTTAGGGTTGATTTGTATTTTAACTAAGTCACTTTATATATCTGGTCCCATTATACA
AGTATACTTTTCCTTAGGATAAGAAAGTGATCTTTATATATGTTTATCAACCCAAATGCCCATCAGTGATGGACTGG
ATAAAGAAAAGGTGGCACATACACACCATGGAATACTATGAATCCATAAAAAAGAACGAGTTCATGTCCTTTGAAGG
GACATGGATAAAGCTGGAAGCCATCATCCTCAGCAAACTAACACAGGAATGGAAAAACAGACACCGCATTTTCTCAC
TCATAATTGGGAGTTGAGCAATGAGAACACATGGACACCGGGAGGGGAACATCACACACCGAGGCCTGTCGCGAGGT
GGGGGGCAAGGGGAGGGAGAGCATTAGGACAAATACCTAATGCATGCGGGGCTTAAAACCTAAATGACGGGTTCATA
GGTGCAGCAAACCACTATGGCACATGTGTACCTATGTAACAAATCTGCACGTTCTGCACATGTTTCCCAGAACTTAA
AATTTAAAAAACTTTAAAAAAAGAACTGTAGATACTGATCCAAAAAAAATGTTCATTAATGGGGGTTAAATGATTAT
TTCTAAGTAGACTACTCTTGAACCCTTGAATCTTTAAGAATTTTCTTTGCTATTGAAGCCATTCAAACTCTATTTTA
TTAAAGCTGTCGTTATTCTAGTAGATTTTAAACAGTAATACCTGAATACATTAGAAATATGCAAATCTGCATTACAT
ATGGCATCTGCAGAGCAGAGGAGTTTGGTCATCTGGACTCATGCTAAAGTCTCCGAAAAATCCGCTTGTCTTAATGA
TGGTTGACTCGCTAATGCTATGCGTATATAGTCTTATTTTAAGTGATTGAATGATGTGGCTAATAACCCCTCTGTTA
GATGCACTCAGAACCTCACCTACCTGGGTCCTCAGCTCTCCAGTGAAATCTCTACTTTAAGTTTATTTTCTAACATG
GTAAGAGCCTTCAGTTTATGTTATGCTCAGGCCCGTCACTGTGAATAAAATATTAGAAATGGACTTTTTTTTTTTGT
ATTTTTTTAATGGATCCCTTGGAACTTTAAAAAAATTATTTATTTGAGCTTTCTACTGTTATCACAGTGTCTCCTAA
GCATGGCCTCCCGTTTTTTGTTGGTAATATAATTCTTACGTTATTCAAATTAGTAACCATTATTTTTCTCATGGCTA
GAATTCTGGAAACTATTAGGAAATCACTGAGCATAATTGAATGGCTGTTTATTTGAAGAGCTATGTCAAGGCAGCAT
AGAGTTGTATTTTCTTGCAGGGGCTCTGGAGTCAAAGAGCCTGGGTTCAAACCTTGGCTCCACCACTTTCTATCTGT
GGGGCATTGGGCGTGTTACATTTGTGAAACTTTTGTTTCTCCATTTGTAAAGTGAGGTTTGGGGGATGATTAAACCA
GATAACTCATGTGAAATATTTAATGGAAATGTATTTGGTAGGGGATTTATTATTTTTAAATTTGGATTGCACATGAC
ACATGTCAGGGATCATGCTATGCATTTTGGATAGAAAGATGGCTAAGATATCATGCCTGACTCTTAAAAACTTACCT
AATGGTAAATGACGAGTTAATGGGTGCAGCACACCAACATGGCACATGTATACGTGTGTAACTAACCTGCATGTTGT
GCACATGAACCCTAAAACTTAAAGTATAATAAAAAAAAAAACTTATAATCAACTGTAGTAGAAAGAGATCTGAATGG
CTTGCCATTTAGCTAGGCACATGGTATATGTGCTTAATTCATACTAGCAGCCACTACAGTTGTCATGATTAATAATG
AGCTTCCAACTGCACAGAATGCTTTTAATCCATAGAAAATCAAATCAGAAACAAGTTTTTGTAAAATTAATGTGAAA
GGAGCAACAATTAAAATGCAAGATTGACATTTATTTTCTAAATTGGTTCTATTTTCTTTCACATTTACAAAATTTAT
AAGAAAATTCTTTATTTCTATGTGATATAAAGAACTAGAATGTACTTTGATGTGAATTATTGTTGCCAGTGCTGTTC
AACTTTTATCCATAATTTACTAAGCACCTACATTTAGACAAAGGCATTATCCATCCCTTTGGGGAGGATTTCAGATG
ATTCATACACAGACCTGGTCTCGAGGAATTTAAGATTTTCTTTGGGGAGGGAAATAAGGACTTTAACCAACTCAAGA
GTACTTAGAGAATTTTCTGAAAATAATTTTATCAATGAAAACTTGTTATATTAAAAGAAACTGTCATTCTGACTTCC
ACAAATCTAGGCTTGAAACTATGGATAACGAGATATTTTCTATTACTCTCACTCACGTCATTTTCACAAAGTGAAAA
GGTACATTTTAACTAGTGAAAGAATAGAGGAAATGGAAGTAGCTCGAGGCAGTGGACGATGATTCAAAAAGACAGGG
CCCTATTATTTGATCAAGTTATGCAACGACTCTGGGCCTGTTTCTTCACCTCTGGAAGGAGGAATAATCTCCAAGCC
CTTTCAGACTCTTTTGGTAATTCACCTCCAGCACATCTTCTAAATGCCAGCATTAACTGTCCTCTGATTTGTCTCAT
GTTTTTCTAGCCCCATGCTCTCCTGTTCGCCATTTACCCTCATGCAAGGTACAAATTACACCCATCATCACAAGACA
CTTGCTCAAGTCCCATTGCCCCCTTGAAGACCTGCCACACCTACTCTCTCAAAAACCATCATTTCCTGAAAGTCCTA
TACAGCTCATTTGGTATTTACAGTGTACTGCCACAAGCCACTAAGCATCGTTTTGTGAATACATGACTTACAGACTT
AGCTTGAGTAAAGATACTTGAAAATGAACACCATTTCTTGGCTATCTTCCTATTTTGATGTACCCTTCAGGCCTATG
AATTTTAGTATAATAGATAACCAATAATTATTTCTTGGTTCTTTCCTGCACATCTGAATAACCCTATGCAAAGTGAT
AGAATGTTTTTCTATAAGGAGGTCCTACACTGGAGATTGTGTATTTCTTAATGCTGTTGAAGGAAGAGATGTGTATC
TAAAATAAATAGACTCTAACAAACATTAATTTATATTTCTATTATCTGTTTTGTGTATTGAGATATCTCACAAAAAT
AACTAAACATTTTGGCATTATTGATATTACATATTTGCCATGAATATTTGTAAATGAAGAAAAATATATATACATCA
GTAATTATCTTGGCAAACTCTTCAATTATGCAATATTGTTACATAGATTACATATCTAAGTGAACACTGGAGTTTTA
ACAATATTGTGTGTTCATAAATGTTTTATTTATTATTGCCACTAATTCTTATTGCCATTTCAAGAACTATGTATAAG
TTGTTCTAAAAACTATTAAAGTATAGGTGACCATGGTCACTACTGCCTACTTTGGTAAAGGCCAAATATGTGAAGAC
TTTTTAATGTGTTAACAAACGTTGAAGGTTTTTTAACCTGTTAACAATCAGTAGGACTCTTGAAATTATTTCCTAAG
AGAGTAAATTTTACAACTTGCAAAGCATGATTAACCTCTTGTAATTATAAACCATCTCTTGTAGTTATGTAGCATTT
TGTTAATGAGCAAAGAACCATTGTGGTTCCTTTTTACATTTCTTAAAATAATTCTCCGTAACCTCATTGATATCTCC
AGTAAATTTAGATAAGCTTTTTTTTTTAAAGGAGGGTTAAAATGACATTTTAAACTAATTTTTCTTGTTAGTTATAC
AGAGTTGAACTATCTGAGGGTTTTATTGACAGTCATAAAAAATTTGTTATTTTCTGTGAAATATAGAGAATTTAATT
CATTATCATATTATTAATTCTGTGGGCCATTGTCTTAATTCTAGAGGCACAAGCTGTTTTCATCCCACTGAAATAGA
GGAATCAAAGTATGTTCCTTGCTCAAAGCACAAAAGTGACATACTACATAGTATGCTTCTTGAGTAGTCGTAAATCT
CATGTGTTAAATTACATCCCAAAGATTTCAGTATGTTTTATGACTTTAATAATTTATGGTAATTTCTAATCTGGCCT
TTGTTGACCTGTCTTGCTTTTTAAATTTTTAGTTTTTCGACAAAATAATTAACATATTTTAATAATCTTCCAAAGGT
GTTTAAAATGGCATTGTATAGAGATAGCTGAAGGCTTTTGAGCTTCTGTGTTGTAAACACTTTCTTAATAAAACATG
AATTGCTACCAGATGATCCAGCAATCCCACTACTGGGCATTTATCCAAAGAAAAGGAAATCAGTATCTTTGAAGAGA
TAGCTTTGTTCCCATGTTTACTGCAGCACTTTTCATACTAGCCATGATATGGAATCAACCTAAACGTCCATCAGTGG
ATGAATTGAAAAGAAAATGTGGTATGAAACAGAAATTGCTGCTTTAATTTATATTAAACACACTCATATTCTTCTCA
GCTGTTAAGTATTGAGTTATAGATTTAAAGAATTCTATTGTGAAGACTAAAGTGACTATTAAAGTAAGAAATTATTT
TTTCCATTATATTTAACTTATTTCATACTTTAATGTTAGCGCCAATGAGCAAGACTATTGAATACAAAAACTAATTA
AGTAGTGGTGATAGTACAGTATATAAGGGAGAACATTCTTTTAGAAAGGAACAATAACAGGGAGCAATAGAAACAAT
GAATGAGTGTAAGGTCACTTAGTGTTAAAACAGCTAAAATATAGTACAAATAAGTTGCGTTTTAATAGTGATTTTAT
ATAATTACACCTTGATGTTTTATTTGTTACAAGAATTGTCCAGGAAGATTTCTCTAAAGACCAAAGGCACTCTTCCC
CTAAATAACTCCAAAGCCAGTCCTGTGTTTCTATAAAAAAAAAAAAAAAAAAAAAAAAAAAAAAAAAGTGAAAATAA
CGTGATGAACATTTTTGAGGAAGTATAAAACCAAAATACTCCACTGCATAGCTGTTTCTGCAGGTATTGTATTGATA
TATTACATTATTCAGCTTTGGAGTCTCCACATCCAATGTTACATCATCACTCTAAATTAAACATGTATAGATAAATG
AAATAAATGAGATAGCATATGAAAATCTCATAGCCCAGCCCCTGCACTATTTAAAATAGAAATACCAAAGAATTGTA
TTCCTCATCTGAAAGCTATTTAGTGGTGGTGTTTCAAATAAAAATTCCATCTACTGCTGTTGCTTCCATTGTATCTT
TTTCTCTGCGGTACTGAAAGAGAAAGAGACCCAGAAGGGGCCTTGTCTGAAGTGTCCCTCTTTTAAGCTGTTGCTGC
TTTAAGCACAGGGTGGACAAATGTAATAGGAGTTTCATAAAGGTGGAATAAACCAGCGGATTACGGTGTGGGTGAAT
ACTTTCAGATGTTAACCAGGAGCTCTGCTTGCATGCTGGGAGTTGCCCATGCCTCTTCTAGATTGAGGCACATTATC
ATGCACAACCTAACTCCAAGAAATCTTTTAAACCACTGGAAATTGAACCCAGAACATGTCTCTAAGCCAGCCTTTTC
ATCCTGACACCGAATCATAGCATGAGCCAGTCTGTCAGGGATGCTGCTGCTCTCTAGGCAAATTTTAAATGTTGAAA
TAATGAATCATGTTTTCTTGAAAACCATGTACACCAAAGAAAAGTTAGTCATTTTATAGATGATGAATATTAACATT
TTCTTAGACAATCTGATAAATTATCAGATCTCACTTTTGGCTCTTTTTAAGACAGTTATGCCTCAGAAATATTAATA
AACCCCCAAGCCCTTATACTGATCAGTATGTTCACTACTAGCTATGAGAAATTCTTGAAGTTCTTGTAATTATTGTA
TTATTTCCTTACTTTCATTTTATTAGTATGTGAATAATATTTTTAAAAATTCTAGTGTATGTCTTGTATATATTTTA
ACAACATGACTTTTAATTAATGTCTTGATAACATTTCTTCTAGTGTATGTTTTCAGTAACATGATTATTAACTGTAA
CTTTAAAAACCTGTGGATTAGATGGGACCATTTTAAAATGTTTTAAACCTGGAAAATCTGATGGCTTTAGGTTTAGT
TCAAGCTATAGATCACCTGTGGAGAATGGAACTGCCAAAAAAAAAAATAGCTGTAGCAGCCCTTTGAGTATTCTAAA
ATAGGGATGTTATCCAGAGCATTGGTTTCTAAAGCTTCCATTATTTATTGATGTTGAGCTTTCAGGATTTAGCTACA
ATATTTACTCAACATCTAAGCCATGCTTTTTTATCAGTCATGTTTTATATCTTTTATAATCAAACTGCTTATCACTG
AAAAAAATATATAAGTTTCTATGTATCTGGAAGAATTCTCTGGTGTTTCTTAGATATGGATTTTGATGTGTGGAATA
AGAATTCAATTCAAGGATAACAGAGATGTTGTCCTGAAAAAAATCGAAGAAAATCAGCTTTTCTTTAACATTCTGTC
AAAGCTCCTGACTATTAGTTTATCAGCACTGTTTTGCCAAAGGTGTCTTCTCTTCTCTTCTTTGAAAAAAATCATCT
GCTGCTGCTACGCCGCAAGTGTGTTCCCGCTGTGCCTGAGAAGATGTGTGGCATAAAAAAATGGGCATGGCCTGAGT
TAAAAGTGCTACATTTAAGCCAGAGCTGGCTTATTTATTAGTTGTCTAATCATAGGAAAATGACAGAGCATGCTTTT
CTCTTGCAATATCCGTTGCTGAAAATTAAACACATGAGCAGAGCTTTCAGAGAGGTTGACTGGCCTCTCAGACAGCA
CCTCATAGGATGGCCTGTGTTGAAGCATCTCCTTTAACCAGGGTCTGTCCCTCAGCATTGGGTTGGCTCACCTAGAT
TGGATTGTCCCAGCAGAAAAAAAAAACCCAAAATTCAGAATCATATCCAAACCGGAATACTCTTTCATTCACATTAC
TTGTACTACCTTTTCAGAAACTGGATACCTGAGTGTGTGAGGGTAACTTAGAAACTTATCTCATGGTTAGAAGTTTT
AGAATTAGAGAGCGATGATCATGAAACGGACTTCATGATCAGAAGCAATGGAGCAAGGAATGAGATGTCTTTGAGGA
GTATTTCCCTGAGGCTGTGGATAACGCTGACGAATAATCCCCACCTTAAAAGTGGGTTGACCACTCTAGTAGCTGTA
AGGTGGGAGGGTTCTTTCTTCAGAGATAAATCTGTGCTCTTCACTTGCCCATTTCCCAGGTTTTCATGTAGGTAGAA
GAAACACCTGTAATCTGAAGACACTCTTCCTTCAGCTTTGTTAGTGACAGGGATTTAAATATGTCTTTCACACATTT
TCCTTAGATAGTTAAATTTCACTTTTCCTGTTTGTTTTTCTCTGAAGGTATTCTAACTCCCCTCCTAATGGACTTCT
AGAGCTTTCTAATTCTATGCAATTTCTGTTGATTTGTTCTGGTAAACTTTGAAGGTAATCTCTGATTCAACTTCTTG
GAGATTCTATCATGTCATCTCTGTTTATTAACTTTATGTTACTCATGGTTTCTTGATGAGGACTCATTAAACATAAT
GTAAGTAGAAAATTATTAACTACATAATATTTACTACGGGTTGTTATTTCTGATAGTAGCTAGCTGTAAGATTCCAA
TTGTTCTTCAAATCTTTGTCTCAGTGATCTCTGTGTAGTTCTTGACTACTTCAAATAACTTCCTAGAAGGATAGGGA
TTTAATAATCTCTTAATAGGAACACTTAACACACTGCTGGTGGGAACGTAAATTAGTTCGGTCGTTGAAAGCAGTGT
GGTGATTTCTCAAATAACTTACAAAAGAATTACCATTTGACCCAGCAATCCCATTATTGGGCATATACCCAGAGGAA
TAGAAATCATTCTACCATAAAGACATATGCACGTTGTGTATGTTCATTGCAACACTACTCACAATAGCAAAGACATG
GATTCAACTTAAATGCCTATCAATGAACAGACTGAATAAAGAAAATGTGGTACATATACACCATGGAATACTATGTG
GCCATGAAAAAGAATGAGATCATGTCCTTTGCAGCGACATGGATGGAGCCAGTGGCCATTATCCTTAGCAAACTTAT
ATGGAAACAGAAAACCAAATACTGCGTGTTCTCACTTATAAATGGAAGCTAAATGATGAGAACATATGGACACAAAG
AGGGGAATAACACACACTGGGGCCTACTGGAGGGTGGAACACAAGTGGAGGGAGAAGATCAGGAAAAATAATTATTG
GGTACTATGTTTAGTACCTGCGTGAGAAAATAATCTTTACACCAAACCCCCGCAAAATGCAGTTCACCTGTATAGCA
AACCTGCACGTGTACCCCTGAACCTAATTTAAAAGTTATAAAATAAACGTATCTTATTTTCAGTACAATACACCACA
GAGTAGAAGGGTTAAAAGAGATTGCTTCTGAGGAGGTGAGATGGGGGTAAGGACAGCACAAGAGCATTTTGGGGGGT
GATGAAGCTGTTCTGTGTCTTGCCTGCGATGATGGCTACACGACTAAGCCCTTGTCAGAACTCACAGAACTTTACTT
CAAAAGGAGCGGATTTTACTACACATCAATTCCAATAACAAATACTTTGTCTTTAAGCAAAGGGATACCTAAATATA
GCGTATTGAATGGATCTCCAGAAAAACACATTTTTCAGTTCATGTTTCAGCCTAGGCCTCATCTCATCCAGGAAACC
TTGTCTTGCTTGCCTTTACATACATGTGGCAATCAGTAGTTTCTTTTAGGGCTCGGACTGAACACTCAATGAACTTC
AATCTTAGCGCTTGTCGTAGCAGATTGACATGGTTTATTTATATGTGTCATTCTCTGTAGTAAAAGGAAAGGATCAA
GGCCATTCACTTTTGTAGTGATTGTGCATGGCAGTATTTGGCACATAGTAGATTATTAATTATGGAACTTCTGTTTT
CACACACACACACACACACACACACACACACACTTCAGAGCTATTTTCATTTAAATATTTGCTTTAGTCTCCAAAGC
CCCTCTGCCTCAACACCAACCCTTCTATCTCATTATTCATCAGCTTTTCTCCTATTACGAAACTACTTAGGAAAGCC
CACTTATTTAGCTTATGATGGCAAAAATAAATATTTGTACTTTTTTTTTTTTTTTTAGTCATCGCTTCATAGAACAG
CCTCTGTCCTCTGCTTATGCCATGTCTGAATATATGCTGGAGGTAAAAAGAGTTCCTGGTTGAGAGCTTCAATTTGA
GAAACTATCTGAGATTACTTTCCAGGTTCCACCGTGGAACCTGTCTGACCTTGAACAAATGACCTCGAACAAGTGGC
TGAAATCTCTTCTATTTCGTCAACTGTAAAATGGGGGAAAACCATGTCTATCTCATGGGGTTCATGTGAAGGTTAAG
AAATTGCTTATTCAGTGTTTAGCACAGTGCCTGATATGCATAAAGCTCCTAGGAATATTAGCTGTTATTGTATTTCC
TTAAAGAAGCCCATAGCTCTATATGCCCTTTCATTATATGTTTTAGTAGCCCAATTTAACATATGGATAAAATATTT
TTAAGTTAAATGATTTGCTAATGGATTGTTGAACGAGTGGCAGACACCCATATTATAGACGAAGGTCAAGTCCATAA
CATACAGTACATTTCCCCACTTTCATTTCCCATTACCAAAATTCATTATTCTCCTGAGAAACTCATTATAGAATTCA
TGTCAGATTCATCTGTGTGTTCCCAGCAGTGCCTTATATCCAGAAATAACACTGAGTCATTGTCTAGATGTAGCAGA
GGTGGAATCCTCCAAAGAGAAGCCTCAGAGTGGCCAGGTTTGCCAAGTATAGGGATGCCTTGATTACTGGCCTTACT
CTTTATGCTCGTGAATTCCTAAGTTTTATTCCTCCTGTAGTCATAGATTGGCTTTTAAGCTACAAGCTGAAGAGAGA
GAAAACCTCTTCCACCTCGTTGGAATATGTCTCTTCAATCCATTTGAGCCAATTTAGGACATGAGACTGCTCTTAGT
CTAGAACCAGTCATCAGGAGAATTCCAGGTCTGATTGACTCGGACTAGCGGGTCAATATCAGGGCAAAAATTCCAAC
GCACAACACGATGTATCAGTAAGGAGAACCTCAAAATTATTTCTTAACGTCCAGATCATGTTCCTATTTTTATATAT
CTATTTTCTCACATAAGTCATTAAAATGATGTACCTGTGCGGGTCCTTTAATGATACTCAAAGATCTTGAATTATAG
GCTAATAACTAACTTAATAAGCTGCAGAAATTAACATTTCTGCTACGTTTATGTAGCATTTTCCCACATGTACTTCA
GAGGCTTGAGAAAAGACCCTGAAATAATGACTGAATAACAGCTTTACTCACTTAATTTCAAATTTGTTAATTCTTCT
GGGAAATACCGTCAACATCCATTTTATTATTTTTCTCAATTACATGTACGTTTCTACATCAGTGGATAAGTTAAGGA
GAAGAATTCCCTCATGATAATTTTTTCATGCTCGAAAATTTTGAATCAATTTTTTATTTTACATTATACTCTTTCCT
AGTCATTAGAAAGGGAGTGGTGGTTAAGATAGGCAAGAATGCTTTATAAGGATACTACTCTCGTTTCAATTCTTAAC
ATCAAAAACCTTAACAGTGTGTAGACTATAAAATAAAATATCTAGGGATCAGAGCATTGTGCTGAACTTTGCAGGTT
TTTTAGTCAATAATATATATGACGTGTTCACAGAATTCTTTGTCAACAAAGTACTTTTGGAGCTCCAGGCCATTTAA
GTTGGTTTTTGTACTTTTTCTTTTTCTTCGGAAGACTTTTTTTGTTCTATTTACCTGGAAGTGTTTCTTTTTTGGTA
CTGTGAATTAAAATGAGACCAATCTACTAGGCAGGAAAAAACCTTAATTAGATTGTTGACACAGACAAATAAGAATG
TCAATTAGCATCTACTGTCACATGCCTCTCCAGACTGCTTCTAGGATGAGTGGCCTCAAGCAGCTACATCATCTTTA
TACTCCTAAAGCATCAAGGAAACTTGGAGTGACAATTCATATCATGAACACATCCACAGTGATGATGATTGTGCTTC
TTCCCCCCCACCCAACAACAAAGGATGAATGCCAATTAATGTATTCAGTTTTTTGCGTCAAAGGCTGGATCACTTGT
GCAATGAGGGTAATCATCCTGACCAGACAGGCCATACAATCCATATTGTGTGAATTAAAGATAATATGCGTGAAACA
CCTTACTCTGGATGTGGTTCATAGCAGTAGCAAAAAGATGAAAACTATGGTATGCTAACATTTTAGAGATCTGTACT
CTATTTTAAATAATTTTATAAAAGTGCATATACAATAAAAAGTGCACGTATCACAAGTATATGCCTCAAAATCTAAA
GCCAGTCATGTAATCAGCATCCACTTCAAGAAAGAAAACAAAACAGTACCCCTGGTTCCTCTTTGCAATCATTAGTC
TCCCAAGAGTAATCACCGATCTGATCTGTGACAGCATAGATTGGTTTTGCCCTACTATATTTTTGCTGAATTATACA
ATATATGCTCTTTAATGTCTGGCTTCTTAGTGCATTGTATTTGTGTATCAGCTATTCTCTTGTGTGTAGTTATTAAA
CAATCATTTTATGGGCTGCATAATATTCCATAGGGTAAATATAACAGTTTTATTGATAACTTAGCTATTACAAATAG
TGCTGTTGCAGACATATATTCTATTACATGTCTTTTGGTATAAGAATTTACACATTTCACATGGGTGTATACCCAGA
ACTGAGATTGCTAAATATTGGGGCACATTGTATACATTTTGATTTAGTAGATAAGATATTGCCAGATATCGTAAATG
CACAGTTTGATAAATATAGAGATTTATACTTTTTCTAGAGAAAAGCCATCAATATCAGTGTATGTGTATATATATAC
GCGTGTGTATATATACGTATATATATACGCGTGTGTATATATACGTATATATACACACATATATATACGTATATATG
TGTATATATATACGTATATATATACACATATATACATATATGTGTGTGTGTATATATATATATGAAACAACTCAGAA
GCAGAAAGATACCCCATGTTCTCACTTATAAGTGAAAGACAAATAATGTATAAACATGTACACATGGACATAGAGTG
TGTAGTGATAAGCATTGGAGACTGAAGTGTGGGGGTGTGCAAGGGAATCAGTGATAAATTAATGGCTACAATGTACA
TAATTTGGGTGATGGATACACTAAAAATCCAAAGTTCACCACTATCCAACATACTCACATAATAAAATTGCACTTGT
ACCCCTTACATTCATACAAATAAAAAATTATTTAAATAAAAATAAATATGTGTATATGTATGCATACATACATATGC
ATATACATATGTGTTTGTGTGTGTGTATATAACTTACACTTAAAATAAGCATGGATGCTGCAATGAATGCTCAATTT
ACAAGGGTTGTCCATCCAAACTTGTGGCAAGTATCTCACCTCTCAAGTTGTTTTCTTTTTTCTTCATATATTTCTTG
CTTTTGTCTAGGAAGGAATAATTTGGCTTGCCTTTCAAGAGTGTACAGTCAGCATGATAACCCAAACACTTAAGACA
CGTGCTAACCCATGTGGATCCCTTGAGAGAAGGAAAACAGTGGTCCTTTTACTGGGCAGATAGAGCCCGGGGCCAGG
TTTCGTGGCTTGAAGATTTCAGCTTCTCTGCGCCTCTCAGCTCAGTGCCTCTGGAAGCAATTTACAACTTGTGAGGC
CATACTCAAAGGCCCTGTTATTAATTCCCCGCCTTCCGAGACCCCATTTCAGAGGATCTCAATTGCTCTCAGAGTGA
ATTTACTGTTTCCTGAATTCCGTAATCCCAATAGCAGGTCTGTTGTCCTCATTAGATAGCTTAAGTTAGAGTCGGCA
GTGTAATTGGCAACTGAGCTACTAAGTATCCAATGCTTATGTGGAAAATATGTTCCCTATTGCAAACAACTGATATT
CATATTCAATTTGGCACCATCATCTATCTATAAAGCAGATACTACTTGTGTTTATTAAGTTTTATCCCAAATAATTA
TTTTAGTAATAATGCTTGAAAATAGGCCTTGGTCATTTGCATGTCTGTATATGGCATATCCTGAGTCTTTGTATGTA
TTAGAAAGATCACTCGTTTTGACTTGATGGTTTAATAAAAGATGTCCCTCACTTTGGGCAGAGACATTTGAAAAAGG
CACTCCAACCAGGGACCTAAGAGGTGAATGAGATGCAGCTCTGAATCAGGTCACACGGCCTCAGGAAGGAAACATCT
TGGTTTTCACATCCCTCACTTCTCGATGTCATGTGCAATACACAAATGACCCCTCAACACACACACAGGCACATACA
CAAACACACACTCACTCACTCACTGTATTGTCTCTTTCCTTGACTAAGTCCTTCTTACTAACTCAAGCTCTAAAGCT
TTTTTACTTACCTAAGGTGAGTGTGTGAGGATTTGAGGTTTCAATATTAAAATTCAGAAACATTTAAAGTTCATTTT
AAATATTAGTAAAAAAAAATCTTGACAAAATACAATTATAGACAAAAAGAAAATTCAGAATATTTGGAATTTAAGGT
TGAGGTTACAGCCCTATTTATGAAATATTAGAAGAAAAATGCTGGAGAGAATAAAGCAGGTTTATGAGTCTGATAGA
AAGCATAACCAGATGATTATGCATATATTTGCATATGCAAAGCTTTCTAGGCAATCTGAACATTTAAACCTACAAAT
GTGGCTGCGATGAACAGCCACAGAAGAGCAGGCTAGAACAGAAGAGGAGGCTAGAACAGAAGAGCAGGCAGAAGTTG
TAAATGAAATGTTAATTTTCAATGGTTGATCTCCCAAGTACTGGAACAGATTTGTGCTGTTTTCAAGGTTTTGGTTC
AAAGAATCCAGTAGTGTATTGAATTGTTTTGTGGCACTTCCCTGTTATTTTGCTTTGTAAGCTACCTCAATCCATGA
AGTGGCTATGAGCCCCTTATACAACACTGTTGATTTTTTTTTCCTTATCTACGCAAAAGATTTTTGATTCAGGGCCA
GGCATGGTGGCTCACGCCTGTAATCCCAGCACTTTGGGAGGCCGAGGCAGGCGGATCATGAGGTCAGGAGATAGAGA
CCATCCTGGCTAACACGGTGAAAACCCACCTCTACTACAAATACAAAAAATCAGCCGGGCGTAGTGGCATGTGCCGG
TAGTCCCAGCTACTCGGGAGGCTGAGGCAGGAGAATCACTTGAACCCGGTAGCCGAGATCCTGCCACTCCACTCCAG
CCTGGGCGACAGAGCCAGACTCCATCTCAAAAAAAGAAAAAAAAAAAAGATTTTTTATTCAGGTGGCTATCAGACTC
ATTAAATAGAAGCCTTAGGTTAAGTTCACGGGTTGCTAGTTGGAAGCCTCCATGGACTATGTTCATAAAATAATAGA
AAGGAGTTATGCAGGACTTCTTGAAATGTTATTTAAAAAGTCAGAATAGGCTTTCTATTACTTGTCTGAGGTCAAAT
ACATGTAGTGCTTTCTGACCATTTCATCCAGGGTGTTAGCTAGGACAATAAGAGGTGCTTAAAAATTATTAGATTGA
GTAAATGAGAAAGCCCTTAGAAACATAGGAACAGAATGACCCTTGCTTTGGATCTAATATTGACTCCCACGCCTAAA
TCCCTTTGGAGAACTCCTTTATTTTCTCTTCCATCAAGAGCAGGTATAAATTAAAAACACCATTAAAGGGGCCATCT
AGCTCAGCTGAAGCTTTCATCACACATGTAGGGGAGGTATGGTTGGGAGGGATCTTTTTATCCTTTAGGTCTTCAAT
TTACATAGGACTTTTGAATAATCAAATAGCCCCAAAGAGCTGATCTTAGGACTAGTTGTAATTGAGACTATTTCTCC
ATGGGGTAGAAAAATCTAGTTGTAGGAAAACTGAGAAGTAGATGTATGTTAACCTCAAAGGCTGTTTTTTACAAAGG
ATGTTAAAGCATCATCTTTGCTCAGAAAGGGAGCAATAAAACAAATGAGTGGAAATAACAAAAGGAAATAATGGCCA
GGTGCAGTGCCTCACACTAGTAATCCCAACACTGGGGGGCTGTGGTGTAAGGATCGCTTGAGGCTAGCAGTTCAAGA
CCAGCCTGAGTAAAATAGGCCTCATCTCTACAAAATAGATAGATAGATAGATAGATAGATAGATAGATAGATAGATA
GATAGCCGGGCGAGGTAGTGTGCCCCTGTAGCCCCAGCTACTCAGGAGGCTGAGATGGGAGAATCGTTTGAGCCCAT
GAGGTCAAGTCTATGGTGAGCTGTGCTCCCTCCTGCCACTGCACTCCAGCCTGGGTGACAGAGTGAGATCCTGTCTC
GAAAACAAAAGGCATACTTTTTAGATGTAATGGAATAGAGTACTTCCAAACCTGGCTGCCTGCTGGAGTTGTATTGG
AAGAGGTTGCACGACTTCAGTGGAGATGGCCTAGATGCCTGCTCAGCAGTCATCTAGTTAAAGCAACTAAGAACATG
TAATATGAAACTGCAAAAAGAGATCGTGTACGTAAAATCACTCTGGGCTCCTCAGATAGAGTAATAAACACAACTCC
TGACAGCCAAATAAAAAGAGAAATAATACAGCCCTTGACTTCCTTGGTTGCTTTGACATACTAAGTAGGTGTTACAG
GTTGGGTTCTCTGGGAAACAGACTCTAAAACATTTTTATTTTTACTTTATTTGTTGTTATTATTATTATTATTATTA
TTTTAGACAGAATTTTGCTCTCGTTGTCCATGTTGGAGTGTAATGGCACAATCTCGTCTCACTGTAATTTCCGCCTT
ATGGGTTCAAGTGATTCTTCTGCCTCAAACTCCCAAGTATCTGGGATTACAGGCAAGTACTACCACGCCTGGCTAAT
TTTGTATTTTTAGTAGAGACGGGGTTTCATCATGTTGGTCAGGCTGGTCTCAAACACCCGACCTCAGGTGATCCACC
CACTTCTGCCTCCCAAAGTGCTGGGATTACAGGCGTGAGCCACTACGCCCGGCCAGACTCTAAAATAAAGTTTAATA
TGCAGAATACTTATCAGGGAATGCCCACTGGACCAATACATATTCAAGAGAGGGCTTAGAAGCAGGATTGGACAGAA
AGAGAAGTTGAGCTGTAATGCAGGCCCAATAACAGCCTTAGTGTTAAGCAGGCTGAGAGATTCAGCAGTTAATGAGA
CAGTCAACCCAAACAGTTTTATAGGCATCAAAAGTATGATCAGCATGGTGTCAGTTTCCTGTGTCACTTGTCCCACA
GTATGATACCAAAATTAAAGAGACCAGATGACATGCAACACAAGCAGTGTGCACTCTGTTGTTGAGAAGCCAATTTC
GTCATGCAATTAAGCAGTTTTATACTCTGCAGCTGTACTTTAAGGGGAGCTGAGATGGAACATCATATGTCTCACCA
TAACCAGAAAGGCAGATGAGAAATGTTCTATCGCCACCTCCCACAAGGTAAGGGACTTCCCTAAAGATACAGAGGTG
GGTGGAATATTGCCTTGGTAGACTTCCTCTCAAGACTGCCTATCTTCCCATGTTGGAAGGATCACAGAGCATTTGTC
AAGACGTGGGTCAATCTGCAGTTGAACTTTGTGTATGTGGCCTATGTGGATACTTATAATATCATTGGGCACCTCCA
TAGAGCTGTTTCCCAATTGACCAAACATATGGGAAGCTTCAGAGCTTCGAATGACCCTTCAGAGTAGTCCTGAGAAC
AGTGAGCCTTACTACTCCTGCATTAATCAGTCATTGGATGATAGCCTTCTCAGAAATAAGTCATGACCTTGTGCAAG
GGGGCTCTTCATGGCTGGGACCACCCCTAAAACTGAGAGCTGAAGGCTGTCTGCCACCAGCCCTTCCACCTGCTGGG
ACAAGTTCTTTATTGAAGGGAAATCTGAGTAGTTCATCAGCGTCCATCACAGTAGTCAAGCCGTTCATTCTTCCTTC
TTATGACAACATTGTGCTTATTGTTATGTAATCCCTTTCCAGAACATTTTAGGTTAAGTTTTAAAAATAATGCATAT
AAATAGACAATTCAAATACTGGGGAAAAAAAGCTTGCACTTATATTGTTATAGAAATGTGCACACTTAAAGAGCTGA
TTTCTTCTGGGTATTTACATAACTTTATTTAAAAATCCATCCATTTTTAATTAGCTGTTTTTAATATGCAGTTAGCT
AAGATATTATAAGCCATATATTAGGCTAATGGACATTTAACAGCTTAGTTAAGTTCTTTTAATGGAAATGCTGACAA
ACCTTTGTCTGTAATTATAGCAACACTGTGATTACAGAAGGAGGTGCCTCTCCTTGTTGTTTGCAGCCCTAAAATTC
CATGTGGCTATAAGTAACAAAGTCCATTATTAGATAAACACAAGTCATACTTGGCATTACTTGCATTACTCGTCTCC
TTGCTTTATTTGAATCATTTTTTAAAGTTGTAAAATGTTTTTCAAAACTCAGAATAGTGGCCAGTTAATAATATGAT
TCCTCTTATATTATGAGATTTTAAAAAATAGTTCACCAGTTTCTGGTGGCCTCTATACCCATTGGCAAGTCCTAGCC
ATTGTGAATTAAGTAAACAATTCTTTATGGAAATTTTTTAATCCTTAAACCCTATAAGTTTTTATTCATCATGTCAG
GTCACTTGTCAAAGGGTTTAACATTCAGAATTCAACAAAAGTTTATCAAACACCTATTACAGGACGTGCAATTTTGG
GCGCACTGGGATTTCAGCAATTAACAATCAAGATATGATTTGTATCGACATGGATATTACATTCTCTCACAGGAGAC
AGAAAACAAAATAACTAGAAAATATACATAAAGAGACTTTAAAATGGGGTAAAATTACAGATTGTGACAGGATGACC
ACTTTGGTTCAGAATATCTAGGACATTTTTTTCTTTTTTTTTCCCCTCCCTCCCTCTTTCTTTTTTTTCTTTTTCTT
TTTCTTTCTTTTCTTTCTTTTTCTTTCTGCCTTTCGGAGTCTTGCTCTGTTGCCCAGGCTGGAGCGCAGTGGTGCAA
TCTCAGCTCACTGCAACCTCTGCCTCCCATGTTCAAGCTTTTCGTGTGCCTCCGCCTCCCAAATAACTGGGACTAGA
GGCATGCACCACCAGGCCCAGCTGATTTTTGTATTTTTAGTAGAGATGGGGTTTGACCATGTTGCCCAGGCTGGTCT
CAAACTTCTGACCTCAAGCGATCCACCCGCCTCAGCCTCCCAAAGTGCTGGGATTTACAGGCGTGACCCACCAGGCC
CAAGCAAGGACATTTTTTTCTGAGCCATGTTATTTAAACAGAGATCTGAATGACAAGAAGGGGCCAGCTCTGTGATG
TAGGGGAAGAAAAATATGTTCCTTCTACCCTTCTAGGCTGCCCAGCTGGAGTCCTACAAAGTTAGAGTGACAAAAGA
CAGATTAACAAGAGGAAAAGCCTAGAAGTTTATTAAAATATTCAGTGCACATACACCTGGTAGAAACTCAGTGATGA
GTAACTCAAAGGGGTGGTTAGAATGTTGGGTTTATATAGCATCTGAACAAAGAACAGTAAACTTGTAGAGAAATGAC
AAAACAAAGAAAAAAGGGGTTTAGGTATTTAGGGTTGCCAAACTGTAGGAAGGTAAATATATGGGAGAAACATGGAG
TATAGTTTGTTTATGCCAAGTCTATCTTGAGATCAACTTTTCGTATTCTTCATGGCCATAACAATTTCCCAGGAGAG
AGGGCTTATAGCAGTTATCATTTCTCAGAAGTTTCTGCTTTTATTTAGACAAGGGAAGCACTGGGAAGGCTTCTTTT
TGCTTATATTGATTCTTACTTGCCTCTAACTAAAAGTAATCTTTATGTCAAAGTGCCATATTTTGGAGTGGTATATA
TTGATCTCCTATAATAACAATCAAAAGGAACAGTATTCTAGGCAGGAGTACCACTAATGCATAGTGTTTGGTGTAAA
GACAAGTTAACATATTCATGGGGCAACAACAACAATAAGCCAATATGGCTAAGACATTGAGGATGAGTGAGTTGGAG
AAGTAGGCAATGGCCAGCTCATATAAAGACTTGTTCGTTTTTATAAATTGTTTAGATTTTATTGTAATTATGGTGGC
AAGTGATTGGAGAGTATTAGCTTCACTTTGACTGGCTTATCGAAAACGGAATGTAGGGGGTGAAAGTGGAATAAAAA
GACCAGTCATTAATTGAGTAGTCCGTGTGAGAGATGATAGTGGCTTGGACAAGGACGATTGTACTGGAGAGATTGAA
GCGACTGATTTCAGATTTGTAGTCAACAAGGCTTAATTGGTAGGAGAAAAAAATAAATCAGTGTTAACTCTTTAATG
TTTAACTTGAATAATTATGATGAGGGTATTACCATTTATTGAGATGTAGAATATTATAAAGTAAGAGCAGATTTGTT
CAAAAAGTATCAAGAATCTTTATTTGGACATGCTAGTTTGGGGATGCTTATTAGAGACCCTAGGAAACTGAATATAA
ATGTGGATTTTAGAGAAGAGCTTAGGGCTGGCAGATGCACATTAAGGATCTGTCTAGAGCCATGGCGCTAGAGACCT
CCAGGAGAACATAAATAGTCTCAAGATCAAGCCCTGAGACACTCAGATGTTTAGAAGTGGAACAGAAGAGGGACATC
CAATATAGAATACCAAGAATTAGGAGGGGAATCAAGAGAGTGTGGCAATATGAAAGATACAAAAAGAGTGTTGAAGG
GAGGGAGTAATTAATAACCAGCATGTTATGAGGGGCTCAGTATAATGAAAAGATAAGTGACTATTGGATTTGGCAAC
ATATAATTTTTTGGTGATCTGGACAAGAGCAATTTGAACAGAATGATGGATATGGAAGGTCCAGAGGAGTAGGCTGA
GTAAATAATATAAGGTGGGAAAATAGATACAAAGATTATAGACAACTTTTTCAAGAAGTTTTACTGTGAAGGGGCAC
AGCAAGCTGAGACAGTGAGGATAAATAATAGACTCAAGGATGGTAACTTTAGAATAAGAAATTTCAATCTGATGGGA
TTTAAGTGTTAGCAAGGAAGCTTTAAGAAGTTATTTTCCCCATTAGAATGATCTGAAAAATGTTTTAGAACATTCCT
CTTATATTCTATTTTATCACATTTATATAACTTTCAGAGAATTGAAAGAGGTATTAAGTTATTATGAAATTTTCTGA
GATTAATAAGATAACAATTATAGGATGTTTTCTTTTAGTTGAAATACACCTACTCAGCCTAATTTTTATAACTTCTT
ACTGAAGTATAATATACTTCAGTAGAAAAGCATGCCTAATATAAAGGTGCAGCTAGATGAATTTGCACAAACTGAAC
ACATCCCTTTAACCAGCACTTAGATTAAAAACAGAACCTTGATGATACCTCAGAGGCCCCCTTCTGCCCCTTTTCAG
TCTCTCCGTGCTACCCCCATGGATAAGCATTATCGTGATTTCTAATACCATAGATTAATTTTGCCAGTTTTTGAATT
TTATGCAAATGGATCTATTTCACCTAATTGTAAATATATAACATTGTCATAGCAAGGCACTCATTGCCTTACACTGA
AAATTACATTGACTCTTTGCCACAAGCTTAGACTTGCTTTCTCATTTTATTATCATCAAGCCTATAGCTTTCACACT
ATACCTTGTTCCTGCTCTTCCCTACTCTATTTCTTGGTAGATATTCTATATCAGTCTTAGAGTGCAGTTTGCAGAAC
CCCTCCATCAGAATCTCCTAGGGAGCTTGTTAATAATGCAGATTCCTAGGCCCCTCCCATGGTTTATGAATCTGAGA
GTGAGGCAGACAAGACTATACCCTCTCATGCCTCTATAATGTAATAATGTCTTCCTAGAATGTTCTTTGCTGCATCT
CTTATTAAAGAAATCTTATGGGCCGGGCAGGGTGGCTCACGCCTGTAATCCCAGCACTTTGGGAGCCTGAGGCGGGC
GGATCACATGGTCAAGAGATCGAGACCATCCTGGCTAACACGGTGAAACCCCATCTCTACTAAAAATATAAAAAATT
AGCCGGGCGTGCTGGCAGGCGCCTGTAGTCCCAGCTACTCGGGAGGCTGAGGCAGGAAAATGGTGTGAACCCGGGAG
GTGGAGCTTGCAGTGAGCTGAGATCACGACACTCCACTCCAGCCTGGGTGACAGAGCGAGACTCTGTCTCAAAAAAA
AAAAAAAGAAAGAAAGAAAAAAAGAAGTCTTATGTTTCCTTTATGGCCAGAGCACAACATTGTCATGAAGTCATCTA
AAATTTCCCACTAGAGGTAACATCTCCTTCCCCTGTCTAGCTCTTTTAAAGCATTACCTCCATTTGCCTTGTATCAT
AGCTGCTTGTACACCTGTCTGTCTTTCCGCTGAGGTTATAATCCTCTGGAGGGTCATGACTTTGCATTCCTTTGTGT
CTCCCATTAGCAGCCAGCACAGTGCCTTGCATACTGTTAGTTCTAAATAACTTCTCTCTCTCTCTCTCTCTCTTTTT
TTTTTTTTTTTTTTTTTTTTTTTTTTTTTTGAGACAGAGTCTCGTTCTGTCACCCAGGCTGGAGTGCAGTGCAATGG
CATGGTCACAGCTCACTGCAACCTCCCCATCGTGGGCTCAAATGATTCTCCTGCCTCTGTCTTCCAGTAGCTGGGAT
TATAAGTGTCTGCCACCACGCCTGGCTAATTTTTGTATCTTTAGTGGAGACGGGGTTTCACCATGTTGCCCAGGCTG
GTCTCGAACTCCTGGTCTCAAGCAGTCTGCCCTACTCGGCCTCCCAAAGTGCTGAGATTACAGGCGTCAGCTGCTGC
GCGCATCCCTAAATAAACTTTTTTTTTTTTGGCATGAAATCTGTAACACTGGAAAGATGTTATTGCCTTAGAATAAT
TAAGAGATTAAATGTAGAATCTCAAAAACATTCATTTTTTTCCATGAAAACTTTACCAGGCCTCAAGGGATAGGAAA
ATTATGGGTACAGAATTGAGAATCTGTAGGAACTTGCAAGATAAACAACGGTTTCACAAGAAAGACCTTGTTGGAGA
GTTAAATTTTCAGACAGTTGTAATAACTTCACATTAAAGTTTTGTCAAAAAATAAGTATCTGCATGTTTTGTTTGCC
TTCCAATGCCCTCATTTTATTTGATTTTTTCCCATAAGTAACTATAGTGAAAGCACGAAAATGTGTTTCTGTGTTTG
TGTGCCTGTATGTTAATTGTGACTGTTTCTATTGCATTGTTATTGCAGAACCTAGGCACGCACTCTGTAGGCTTGGG
TGCTTTCTCCAACTGAAAAAAATCCTACATATGGATAAATTATTTTTACAGCCAGTGTTTAATTTTACAAGTGGTCC
CCCTCCTTCTGTTTTTAGGATGGCAGAGAGAATACATATTTACTTACCATTATCACTTACTCATGCTTTGAGCTTGA
AGGAAATGAGACAGAAAAATGAAGTAACATTAACTTCTCTCTGGAACTATGTTTCTCATATTAGAGCTTTATCTGAG
GAGTTCACTTCCTCTCTCTTCAATGCTTTGTTCCTCTCCAGTCGATTCAAATGTCCTCTTAAAGCAGAAGTTCCGAA
CCTCTTTCTGTGACTTCAGGAGAGCATGAGAATGTAAATATAAGTTTTAGGACTAAATTTTCAAAGACTTTTTCCAC
TCAGCTCTCTTTTCCTCTTCGGTTTGTTGTTGTCGTTGTTGTTGTTGTCGTTGTTGTTGTTGCTGCTGCTGCTGCTG
TTTTTCCCCTTCCACTTCCGTAACTGAGCTCTTAGGGTCCATCTGGAATCTGATTGCAATTAAAAAAAAAAAAGTTT
ATTTTTACCTCCTTGTACGTGCTTTCTCCTAAAGCAGGAGTCAGAAGCCTTTTTTCTTTGAAGGGCTAGTTAGTAAA
TATTTTAGGCTTGTCGTCTTTGTCGCAATTACTCAACTACGCTGTTGTAGTATGAAAGCAGACAATACATACCTGAA
TGAGCATGGTTTTGTTCCTAGCAAACTTTACGCACAGAGAAATTTGGATATCGTATAATTTTTATGTGTTGCAAAGT
TGTATTATTCTTTTGATTTCTCCCCAACCATTTAATATGTAAATCCCATTCTTAGCTTGTGTGCCATACGCACACAG
GCAGCAAATGCGAGTTGTCACACAGGCTATAGTTTCTGACTTTATGTCTTAAAGTAAACAGTAATAATCATTCTCTT
TTTCCAAACAGTCCACTAATCTCCCTTTGTATTCAGCCCTTGCATAGTAAACGCCGTTTCTTCATCATCCTGATTTT
TATTCTGAGAAAATACTGTATATTGTTCCCATGCACTAGGGTTCGGGGAAATTTAAAAGGATGTAGGATCTCCTTTT
CATTGGTCCTAAAATTGCACTGGGGAGGCAGGTCATGTTTATGAACAGATAAATAGTATCATAATATAATCATGCAT
TTCTATGGCTAGCATTTAGAACTATAGCTTTTGATGTCATGTGGTTTTTATATGGTTGATTATTTTTTTCTTATTTA
TAAAATGAAAAAGTTTGAGAATTTTTCATCTCCTTAATGTATTCCCTTATTTGAGGGAAAAGTATTTACCTACTACA
TAGGAATTTATCTTAAAATTTTCTTTGTCTATCTATTTTTATGGAATATAATCGAGCAACTATTTTACTAATTAATA
CTTTAATATCATTATGAAAATGTTCTCATATTTTTAACCTTATAAGATCAGATAATTGCTATGCCAATCTATGGTTG
AAATGGGTTCTTATACTTAACGCTATGCTCTTTCTTCTGAGATGTAAAAATATGTTTAAATCAGAATTTATATAGGT
GTCAATTCAAAATGACAGTAGTTCATTATTTTGATTAGTATAAATGTTCACAACTAATTCTATTCTCTTATCTATTA
AGTCACCAAATAAAGTATATTTGTTTTAAATATTTAACAGTTTAAATTATTCTTTGAAAACTTATGAGTCTAAAGTA
AGAACAATTAACCCATTCATTTTGCAAGTGGGATAGTTGAATTTTACTTGCAATCCAGGGATTTTTGACAGTTTGAA
ATATACATACATACCATGTATGTTTAGGAAAACATTTAAAAAGAGGGGGTTGTAAAATAATAATAGTTCTTCCATGA
TTTTTTAGCCATAATGTTTATAATATAAAATATGTATACTCTTGTTATTGAATGTAGTATGTTTCTAATTTACCAGA
AGGCAAGAGAATAATCCTGGAGAATTTCTCAAGGCATCTTCGAACTCTTTGATTTATTGCTCACATATAGTAATTTG
CCAAATGACGCCCTAGTGAACTGAAAGAATTAATGCCCCGTCCTAAGTCACTTTCACCGAGGGACTGAAAACCTGCA
GCATTTTGCCAATTAGAGGAGGAAACAATCTACCTTGCAGAGTCAGGAGTACTGGATAAAGGAGCTAAGAGTGTTGC
TTTTTTTCCCCTTCTTACTTTAAAAATCCCAATTCATCCCATGTCTTTCTTAAAGGCTAAGTGAAGTAGTAAGTACG
TTTTTGCAACATACGAATTTAGCAGACTGGCCTTGTGTTTATTTTTGGCCGGAACCATTACACTTATTTCCAACCCT
CTCCTTTATTTGTTGGTTGATAATGGGCTAATTTTGAATCTTTACTGTCAAAAGAACATTAAGAGAAGCAGCCCTGC
CTGCATCGCAGGCTATGTCTGTCCTTTGCCGAGTATTAAACACTAAAAAAAAATTAAGAAAATACTAACAAAATGAC
AAAGCATTAAGAAAATAAAACTAGATGTTAAAGGAAATGAGAAAATAGGAAAGGATGCTGTACCTGGAGTGATTTTT
TTTCCCCAGGCTACCTAAGATGATCAAAAAAGAGCTAATTTCTCTTAGGTTTCTATTAAGGAATTACTAGAATATCG
GGCACACCAGGAAACTTTATCAGTGGACCTGTCCTGAACCAAATTTTCTTAATGTATATATGATAATTTGTTACCAC
ATCCCAGATTATTTTACAGGAATTAAAATATATTTGAAACACTGACAGGGAAAATTGGGTAAGACATTGATAGATAC
TACAATCTGTACTTGAAACTGCACTCAAGGAATTCGTTAGTCAAGAAAGAACACAATGACTGTGGGCCCCTCTGGGT
TTTGGAACCTCTTTTGTAAAGCATTTTTTTTTTTCCCAAATAGAAGATATTATTTTTGAAAAGGTTAAATAAAAAAT
CTTTGTTCACTATATAGTTTCCTCCTAAGGAGTAAATTAATTTATATAAAATATTGCAATATAAATAACAATTTTAA
AATCTCAAAAGAGCAGTGTTTTAAAAATAATGTAGAAACATTAAGAAATGACTTCAAATGATAAGAATGTCATTGGA
GAGCAAAGGGTTTTTAATATTACATATCGTGGCACGTATATCAGCACCCAACCGCTCAAGATACAGAGTTCTTTACA
AAAATCAAACAGAAGGAAATGTGCCACCTTGTTCATAAACTATATTTAATAATAAGCCAGGCAGATAAAGTCACTTT
CACAAATAATGAGCAAGCCCATGGTAATATAATTCATTTACAATAAGATTTATCTCATGGAATTCTTAGACTGTGCT
TTGAAATTTAAATAATTCTGATAAATGCCAACAGAATAGAGAAATCAATTCCAGAGCAATTACTAACACGTTGCATT
ACCTTTCTAACATTAATATTTCTCTTCATACATATCATTGAAGAGAAAATGAGGATGGAAAATAAAAAGATCAGGTA
ATATATTTGCTTTCTCATCTAGGGTTGTTATGATCTTCAAGATGAAGTTTTATTTTTTACTCCTAGCAAATGATATT
CTTTTTTATTTTAGTTTTTATTATTTTATTTTTCTGTAAATTATTGGGGTACAGGTGGTATTTGGTTACATGAGTAA
GTTCTTTTTTTTGATATTTCTGAGATTTTTTTTTTATTCTACTTTAAGTTTTAGGGTACATGTGCACAACGTGCAGG
TTTGTTACGTATGTATACATGTGCCATGTTGGTGTGCTGCACCCATTAACTCGTCATTTAGCATTAGGTATATCTCC
TAATGCTATCCCTCCCCCCTCCCCCCACCCCACAACAGGCCCCGGTGTGTGATGTTCCCCTTCCTGTGTCCATGTGT
TCTCATTGTTCAATTCCCACCTATGAGCGAGAACATGCGGTGTTTGGTTTTTTGTCCTTGCGATAGTTTGCTGAGAA
AACCACGAGGTACCATCTCACGCCAGTTAGAATGGCGATCATTAAAAATCAGGAAACAACAGGTGCTGGTGAGGATG
TGGAGAAACAGGAACACTTTTACACTGTTGGTGGGACTGTAAACTAGTTCAACCATTGTGGAAGTCAGTGTGGCGAT
TCCTCAGGCATCTAGAACTAGAATTACCATTTGACCCAGCCATCCCATTACTGGGTATATACCCAAAGGATTATAAA
TCATGCTGCTGTAAAGACACATGCACATGTATGTTTATTGCGGCACTATTCACAATAGCAAAGACTTGGAACCAACC
CAAATGTCCGACAATGATAGACTGGATTAAGAAAATGTGGCACATATACACCATGGAATACTGTGCAGCCATAAAAA
AGGATGAGTTCACGTCCTTTGTAGGGACATGGATGAAGCTGGAAACCATCATTCTCAGCAAACTATTGCAATGAGTA
AGTTCTTTAGTGGTAATTTGTGAGATCCTGGTGCACCCATCACACGAGTAGTATACACTGCACCATATATGTTATCT
TTTGTCCCTCGGCACCCCTTTTCTACCCCCCAAGTCTCCAAAGCCCATTGTATCATTCTTATGCCTTTGCATCCTCA
TAGCTTAGCTCCCACGTATCAGTGAGAACATATGCTGTTTGGTTTTCCATTCCTGAGTTACTTCACTTACAATGATA
GTCTCCAATCGCATCCAGGTCATTGCAAATGCTGTTAATTCATTCCTTTTTATGGCTGAGTAGTATTCATATATATA
TATATAGACACACGTACATACATATGTATATATACCGCAGTTTCTTTATCTACTTGTCGATTGATGGGCATTTGGGT
TGATACTTGCACACACATGTTTATAGCAGCATAATTCACAATTGCAAGTGATATTCTCAGGAAGCATGATGTAAGTG
ACAGAGACTTACTTTGTAGACTGCACTCATTCACTTGTTCTCTGAATGTGCTCTAGGCAGCCTGAGTTTCTACTATG
TCAGTGTTACATAGATGAGAAACCCCATGGGTGGTTTCCACAGAGGCTGCAATACTATTTTTGATACCAAAAATCTG
TTTGGTTTTGTGAGCCCCAGATGCCCATATGGAAAACTGAAGTGTTGATACCTCTTTGTAGCCCTCTGATGAACTGC
ATGGTTCACCTTCCTCAGCAGTTTGAGCGGGGTGGGGAGAGCGCCTGCTTCCTAGCCATCCGATTGGCCTGAATCAT
CAAAAATGCTATCATGAAACAGGTTCTGTTTATCTGCTCCAGATTACACCCATCATGTTCTAGAGTGCTGGTTTCAT
GCTTGAATCTAGATCAAGCCTGCTTTCCTCCCCTGCCTGTACTCCCTGTGGCTACCTACAGTCCTGCTGCTGACAGA
TAATCTAAACCAATAGCACCTAATTAGCCTATTTGCTCATGTGTTTTTTCCATCGTGGTATAATGTCCTCCTTGTCA
ATTTAGGGTGAAAATGTAGCAACACGTTGCTGATGGTTTAATTTCTGGAATGCAGGTAATGAATGTGTTTTTGCTTA
TCCAAGTCTTCCCATCAGATGTCAAATATAGAAGAACAGTGTTCAGAGGTCCTAAATTTAAATTGGAGTGAGAAATT
CACAGCGCCCCTGAACTCAGGCAAAATGCACTCTGACAAGTCAACCAGATATTCACAGATGGTCTGGAGGATTTGAA
GCCTAATTTGGTGAAATAAAATTAAATGAGTGAAATTGTATGCAGTCATTAATCTATCACCATACTTAAAATGCTTC
ATTGAAATTTCTTTTACTGCTTCAAATGAAAAAAGATCAAACTATGTTATAGAAAAGCATTCAAAACCCTTACATAA
CATAGATAAAACTTGGTTGGAGACTTACAGAACTTTCTCTGCTGCTTCGAGAAAGTTACAGTGCCCACAAATCTATT
GCTATTAGAATATTTTATTGTATTCAACACTCAATTCTACCATAATTATGTATATGAGAAAAATATTTTTACCTATA
AAATAATTATTATTACCTTTTAAAAATCTGACATTCTTCCTTTTTTCTAAAGAAACATATTTAGATTTAGCTTTTAT
TTTATTTTTGTGTTGATACATAGAGATTGTACATATTTCTAAGATTCTAGTGATATTTTGATACAAGCGTATAATGT
GTAATGATCAAATCAGGGTAATTGGGATATCCACCATCTGAAACACTTATCATTTCTTCTTTTCAATGCCATCATAC
CAAAAGGAAGTAAATAGAATTTCAAATATAAGGACAGCCATGATTTTACATACATGCCTACGATTCCACCACAAACC
ATAATTACGTCCCCCAAACTTTTAACATTTCAGATACTTTGTCCCAGGTATTTCATGATAAGGATTGGGCTATGACT
CTGTTACAGAAGGGCCAAATGACTAAAATGTCTCTGAACAATATTGATTGCAAATATTCTACCCAGTTGTCAGGTCA
ATATGTTCCAATTCGGAATTTATAACATTGTATCTCTACTCCCAAACCATCCAATCTCACCTACCTCACTTCCATAT
TATGGTGGGTGATCTCAGATTATATTTAAGCTCATGGTTACTTGTCAAGTAGATATGGAGTTTAGCCTAACTTTTGA
AATTTATGCTGAGATTACCCTTCTCATTATAGAATTAAGTAGGCAGTTTCCAAGTTTAGATTTAGCAGGCAGTTTTT
TTCAAATCACTTAAAAGTTATATTTTTTTAGGGCATTGAACAGGTTTGAAATCCTACCAAGATGTCATGTACACATA
GACCAATAGAACAGAATAGAGAACACATAAATAAAACTGCACAGCTACAGCCAACTGTTCGTCGACAAAGTCAACAA
AAAAATAAGCATTGGGAAATGGATTAAAGATTTAAATGTAAGACTTCAAGCTATAAGAATCCTAGAATAAAATCTGG
GAAATACCATTCTGGACATTGGCTTGGGAAAGAATTTTTGACTAAGTCCTTAAAAGCAATTGCAAAAAAAAAAAAAA
AAAAAAAATGACAAGCAAGGACTTACTAAAATAAAGAGCTTCTGCATGGCAAAATAAATGATCAACAGAGTAAACAG
ACAAACACCAAATGGGAGAAAACTTTTGCAAGTTATGCATCTGACGGTGGTGTAATATCCAGAATCTATGAGGAACC
TAAACAATTGAACAAACAAAAATCATAAAACATCATTTAAAAAATGGGCAAAAGACATGAACAGACATTTCTCAAAA
GAAGATATACACGCAGCCAATAAACATGAAAAATGCGTCACATCACTCATCATCAGAGAAATGCAAATCAAAACCGC
AAGGAGATACCATCTCACACCCGTCAGACTGGCTTTGTTAAAAAGTCAAAAGACACCCAATGCTGGCAAGGCCGCAG
AGACAAGGGGATGCTTATACACTGTTGTTGGGAATGTTAATTAGTTCAGCCACTGTAGAAAGCAGTTTGGACATTTC
TCAAAGAACTTAAAATAGAACTATCATTTGACCCATCAATCCCATTACTGAGTAGATATCCAAAAGAAAACAAATGG
TTCTACCAAAAAGACACATGCACTCACATGTTTGTCACAGCACTATGCACAATAGCAAAGTAATGGGATCAACATAG
GTGTCCGTCAACGTTGGATTGGATAAAGTAAATGTTGTACACATACACCATAAAATACTATACAGCCACGAAAAGAA
GAAAATCATATCCTTTGCAGCAACATAGATGCAGCTAGAGGCCATTATCCTAAGCAAATTAACATAAGAACAGAAAA
CCAAATACTATATGTACTCAGTTATGAGTTGGAGCTAAATGTTAGGTACTTATAGAATTGAAGATGGCAACAGTAGA
AACTAGGGACTAATAGAAGGGGAAAGGAAAGGGGGAGACAAGGGTTGAAAAGCTGCCTATTGTGTACTATGCTTACT
ACCTGGTTAATGGGATCATTTGTATCCCAAACCTCAGCATCACGCCATATATCCAGGTAACAAACCTGAACATGTAC
CCTCTGGATCTTAAAAGTTGAAAAAAAAAGATGTCATATAAATATTCGTGGTCACTAAAAGTATCTAATGTATTATA
CATAAAAATAAAAATTGGGTGAATTGGAAGTGTATTCTTTGTATCAAGTCATGTCGGAGATCCTATTCTGCTTTGAT
CACAGTGTGAATTCTTTTGCATTTTTGTTACCAGTCACTTCTTTATTTATTGAACTAATAATTACATATTCTGATAA
TCTGTCAGAAAGATAAAAACATTCTTTGTCCATGTGTCTGAAAATTTTTAACCTATTTTTCTAATGTTTTAAGTGAG
AAGAGCATGTTAATACTGAAATTGTAAGCAGTAGACTGAAAAATCATCCCAATCCATGGGTTATATATTGAATTGCT
TTTAACTGTATTACTAAATATTAAGCTTAATTTATTTTATTTCTACATATCCCCATTTCCACTATAGGTGATTTGTA
TGAATTTAGGAACTTCCTTCTCTCATCCATTTTTATATTAAAACTCAGACTTTCTAAAACAATATTTCTATCCATCC
ATCGTTGGTAACTATGTACTGACATGTTTTGTGCATCCGAAAAATGTTAGCATTAGTTTGTGCGCACAGAAGTAATT
CCAGTCACCATATGATGAGCTGATTTATTTATTTCGTAAGTGTGTTCATTATTATTATCTCTTCAGCACCCAAATAT
ATAGGGGACTTAATGATACCTACAAGTAAAAACGGAAGACAAAAACGCCCTGCTCTCTACAGAGGTTAAAATGTTTT
TGCAACAGGGCTCTAGATCTCAGCTGTGAAAGTAGGGACGAGATGAGGCTAGGCATGCAGTGTCAGTATAATACAAT
ATAATCAACATGTCAGCATCTAATGCAGGTGTTGCAAAACAAAATGTACACATGGGTAGTCAGGTAACAGAAAAGCA
TGAAGTAGTAAGGGCTATCTATGCAAGAGGTTCCAAGCTGACTATATACTGAAATATTTAAACACTATGTGGGGCAA
ATAAAATGGACATTAGAACAGTTCGATGGTCAGTTGGGGACTTCTGCTCTTTCTTCCAGTCTCTGAACATATCTTAA
AGCCACAATCATCTATTTTTATTTATTGTTATACATTTATTTATAAGCCAGCACCCCTGTGATTTAAGTTCTGTTGA
AATGCTGAGTTGGAAAAGATCGATGGATGGGGGAAATTTAGTGCAGAGGTTTTGCCCCAGGTTCAAAATCCTTTATA
AAATATTAATACATGGAACAAATATTGAACAATTAAACCACTGATAAGTTAATCAATCTGATTCAAAGTACACCTGT
GAAGAGGGACATGGCAAGAAAAATATTACAGTAAGAACTAGAAACATTCCTTCATGGCTGCTTGATATGGATATGTC
ATGTTTAAGAAAATTCTTCTTTAGACTGTTGAGATTTTTTTTCCTGACAAAGAAGATTCACTGTCGAGGAAAGAAAG
AGGTACTGTGAAATTTGTTATTGAAAACATGCACATACTTTTGTCAGAATGAGTTAAAGAGTGAACAAAATGTGCCT
ATTACTTACGTGTTGTGCTGTTTTAATTCAAGATTAAAATATTTAACGTCCACAGACAAGACCACTTTTATATGAAT
ATTATTTTTCTGCTTTATTGCTCAATTTTATTACCATTTCAAAACACCCGTGTTGCTTTCTATGGCCAAAGATGTTT
AGCACTTTTCATGGTTATACTTCTGTACAGTCCAAAATACAACACTTACTTTACACATACACAAACATCCAATGTAT
TTTGTTTTCTGTCAAGTAAAGACAATGTCTGTGTTATTAAGTTAAATGTCACTTTCAAATACAGGATATGTTGATAT
TAGAATGTTCAACTTTATTTCCTCATTTAAGCAAATTACAGTGTGAAGAATGTAACTGCAGCAATTTATAAAAATCA
TATCACATTCAATTATGAGAGCAAACTTGTTTTGTAGACTTGAACTAGTTTCAATTAATCTTGGAGTTATCATTTCA
AAAATTCTAAACAGAGAGAAATACGGAGTGTAATAATGGTAGGTCTTTGGGTAAGCTGCTTCCAGGAAAAGAAAGCA
ATTATATATGTTCACATAGCACTGACAAGGAGAAACAAAACTTTGGACGGCAAAGAACTTGCATTAGTCTTTTTGAC
ATGTTCCTGTGGTGTGATTTATTACGTAGACAATCAGCTCAACTTCTCAAGTTTGATATCCTTGGAATCATTTGAAA
TTTAAATTTTAATGAAAATTCATTAATTCCAAGGCCAAAAGAAGTGATTCTAATTGCTTTTGAGAATCAGACTATGA
AAGAATTCTTTGGCAAACTTGCACTGTCTTTTCTCTTTTATCATTGGTTGCTTCGTAGGTACTTAATTGAAGGTCCT
CTGATTATCAGCACGGGCTGACATCAGTTCACTCCATGCATTTTAAACAGTAGGCCAGATGTTTAAAGGATCAGCTG
AAGCATCGATAGCATGCTAGGGTGAATAATAAAATTTTCATTATCTACAAGAAGCAAATAAAAAGCATAAGCATTTT
CCCCCATTATCCTGAAGGAGAAGATGAATGCCTAAGCAACATTTTAAGAATGGGTTGAGTGTGGCCTGTGGGAAAAT
TTGGGTAGAAAACTTGTAGTTAGCTAATGTATATACTGTTTGCCTCTTTAGCTCACCATATACCCACACACATGGGC
ATGCATGCATACAGACAGACACATACAATACACACAACAAACAGGAAATTCAGATATACTGAAGAAATGTATTTAAG
GGATTACTAAGTTTTTGTAAATAAAATCCTTTAAGATGCTGAGAAACAATGGAAGAGAAGTAGGACATGATGGCTCA
TACTTTCGTAATTTACTTGTTTAACGTTTGCCAAGGTTTAAATTAATGTAGATGTTTTTGTGGCTAGGATTAATGAT
CTAACAGTTTGGAATAATTAGGCACTTTTATCACCTAGAAAGCCCAGAAACCCAGCATGCAAAAATTCTGGTATGTC
TGCATTTTACACTTAGATATAACAGAGAAATGACAAGTAGTCAAGTGGATAGAGAAACGAATGATTCTTCACACATG
CACACACACATAGAAATTGTCTTTTTAATAGTATTTTAATGTAACACATTTATGCATAATTTCTCCATAGTGTTTAT
CTTATAGTGAATATGTGATGAATAGTCTCTAACATTAGTGGTTTTATAGATTAAACATAATTAAGGCTTTATATATT
AAAGAGTCAATTGGTGACATTCTAATATAAACATGTTTATCTCATATACATTGAAATATTAGATAATTCATTCGTTG
AGAATAAATCGAATGAGTCAAAACTTTTAACCTCCACTTTGAGCTTTGTAATAGTATCCACTGAAAATATTCATGAA
AATTTTTAAGTCATTTCTATTTATATATTCAGTCCAAACATCTCACAAGTTTAAAATGTAAACTCAAGAATATAATT
TCTGTATTCTACAATTGGAAGCATCCATCATATCAGATGAACTTATATAGTTTGTGAAATTTTGCAAACTTTCTGTT
TAGTAAATCTTAATGTCAAACATTTTAACTTCCAGGTTGTCTTTCTTTTCAGTTTTAATATCCGCGATCTTTGTATA
CTCGTTGAATGGATTCTCAATAAGTAACCCACAAATATATATACATACTATGTACCTACAAAAAATAATAAAAAGTA
AAGAAATCGACACTTATCCATACCTGTCCCATAGTAATAAACTATTCATAAGTATATTTGAAAGATATGAGAATCAT
AAAAGTTCGTGTTTGCACCCTTTTGTGCGTGGAATCCTAGGTTTGCATTTTGTGGATCTAGACTTTTTGGAGTGTGG
AAATAAATGAAACAAATAATCGAGACCCAGTCTTATATTCAGGTTATCATTTTACTACATAAAGCATAAATAACATT
TGCAGTTTGTTTCTATGGCTAGCTCTAAAGTCTTAGCAACGAGAACATTATAGAAAGACTTCAACTGTAGCTTCCAG
CAGAACTTCTGAGGTTCCGTTTATGGACTAAGCAGCAGTTGAGGGGGACAAAACTCATAGGCAATTGATCACTCCAA
AGGATAGATTGTCTTTTCTAACCTAATCAAAAGATTTATAGTGAAGGCATATTCAGATTTTGTTGAAGGATATGGAT
ATATAATCATGTGTGTGTGTGTGTGTGTGTGTGTGTTAGACATACTTAAAACATTATTTGAGTAGAAAATTCTGCAC
AAATGGAAAAGTATAACATGTGTTATATCCACACATGTTGAGCATTTACCTGGCTGAAACATCAAAAGCTGAATTGA
CTTAATTGAATGTTGAATACTTAATAGTTACTTTGTAGTGACTCACTATTAAAACATTATCTCAAGCTTTGTCAGAA
TTAATTTTTTTAAAAAACTCAGATTAGTGTCAGGTTTACTGAAACAGCAGATCTGAAATTACTGTGTTTTTTTTTCC
TTTCAATAATCAGTTTCTAATCCAAAATTGAATATCAGTTCCAACTCTACATTCAGTTTCTGTTTTACTTGTTTGGA
CTGGCTTTTGGTTCTGTTTTCCACATAGATCCTCTCTGTGTAAGACAAAGCCATTTGTGCAGATTAAATTTTACTGA
GCGTGTTAACCTATTTAAAACATTCATCCAAAAAGACTAGTATGAATTCTTCATATGGCAAGCTGCTTGTTTTAAAA
CTTCCATTTATTCTAAAATCCTTTTTACTTATACTTTTTAAGAAACGTATTCCCGATATACAAAAGTAACACATGCT
CATTAAAACAAATTAAAAATAGTATTGTATAAAGAGCTGATACATTTCTGCCTTGCCCCATTTAACTTTCTTAAGTG
TTCATGTGAATCATCCATTCACATCAAGACATTTATCTGTATTCATATGAACGTGTTTTAATATATATAACATATAT
AGAATTTTATATAAACTTTCCTTTTAAAATAGAAATGAAATTATATGATATATTTATTCTGTGTCTAGCTCTTGTCA
CGTAATTATTCAAGAACATATTTCTAGGTTAATATCTGTATTCTTAGGTAGCATTCACTAACTCCTCATCTACTTGT
TTTCTTCCATTCTAATTGTGTTTAACATTTCTTCATACAATTGGTTGTCATTTGGTCTTCTTTCATGGAGGGTGCAT
AATGTTCATTCTCACCAATTCTTTACACTTTACATAACTGCTTGATACGAAGCCAGACCTTATAAATATCAACAAAG
CAGGAACACTGTAATCAGCTATCAGTTTCAGTTGAGCTGAATGACCCTGAATATGTGTACACATATTTTCCAGGAGA
TTTTAAAACTGACACCTCAGATTTCTAAGACCTGGAGAAATCAGCATGAGAAACATTGATCTATATTATTCCGTGAA
ATGATTTCACTAAATAGTGAAGCATCTCCCACATGTGGACTCTGTAATTTATTAGAATAAAGAGTTCATGTGCTTCT
GAAGAACTTGAACTACTCTTCTGGCCTCCGTACATTGGTTTCTTAGCTATAGGAAGGCTGAGCATGTTTTTCCTATG
CGTTTCCTTTCTAGCTCATCATTTTAGTGACAAAACAATCTTTCGTGGTGTTGCTCTAGCTATAGAATTGTTTCAGA
TTCATTTGACCAAAGGTGGCAAATACAACAGTCCCAACAAAAACAAAAGACCTATTACAGAATGATGGAAATGACCC
CAGGGAACAATGGCACCTCCACATTTCTTAATTCCAAGGTTATAAGCAGTGGTGTGGACAATTCTCAATTCCAATGC
TGAATCGCCTTCTAATTTCAAATACCTGTGCTAAAAATTATTTACGTCTACTGAAATAATGAACTGGACCCCACCAG
GAATGGCCGATATGCTTGTAGTCAGAGCACAACTGTAGAAAGAAAATAACATTTTAATTTATAGAGGTATGATGATA
GCTGTTTCATACTGTTTTCAGAACGATGAATGGCCTGCTCAGTAGTTTCTTGTCATCGTACTGAGACACTTTAATTT
CTTACCAGCTGAGATGAGGAATACGAGCCCAGTGTGCAGGTGAAATTGGTTAACAGGAGCCATTAAAATTTGGAAGA
GTCAGAATAGCATCAATCAAAATGCTTTCAGTGTAGGAAGTAAACATGTACTAGCCTGACCCACCTGTCTTTTCTTT
TAGGTATGTTGGTAATATTACAATCATTTTGAGGTATCCATAAACAACTGCTTAGATCTGAAGAATTGTATATCTTT
CTTTACTCTGCCCTGGCCTGGGGTTATGGTTCTCATTGAGCTCTAACCTTTCAGAAAAAAAATGTAGAGAAGTGGTT
CAAGAAGAATGCTTTATCTTGCTTCATAAAAATGATAGTGATAGTTTTATTGAAGGCTTACTATGTGCCAGGCCAAA
GTGCGTTTTATTATCGTTCCCATTTTCCAGGCAAAGAAGCTGGAGCACAGAGAGGCTAAGTGAGTTGTCCAGGATGG
CTCAGCTAACATGCTGCAGTTGGGATTTGCACCCAGACCAACTTCTTTTCAACCACTGTCCCATCCTGTGTCTTCTC
TACTCAAAAAGTGTTTCAGCTCCAAACCTGAAACTTTAAAGAAAAGGAAATCCTTAGTGGAAAGACTAGGTTTTAGT
CACAAATTATCTCCTTCCTTACATTATTTGTCTCTTTTTCAAATACTCCAAGCTTTGATTAAAACTGTCTATCACTA
GGAACATTGTAGAATTGCTAAGGTGGAATTGTTAAAAGAACTCAATTCCAATTAACTTTGCCATTGATTACTGTGTG
TTCTGGAGGGGTGTTCTTTCTTTCAGGTTAATGATGCTTTATTGTATATCTCAAAGATTAAAAATAACAATGAAGGA
AGTAGCAAACCGGAACTTCTCTCACAATGCATCTTTCAATCTCGTGCTTTAAATGAAGATAAAATCATGGCTGTGGT
AAGGTTGCAGGAAGGATGATATAGATTAAGTTTCTTGCAAACTGCCCTCTGAATTTTCAATAGCTGTAGAAGGTATT
GGTTTTCCAAAAAATTGACAAATTGAGGATTCATTCAGCAGTTTTTTTCTAGGTCTCTTACCAGAAAGTGATCACTA
AAAAGTGTAGGGAAACCACTCAAAGTTGGATAGATCATTATTTTCACTTAAGCATTTTAATTTCTTGAAGGAGCTTT
ATAATGCAACAAAGAATTTACAGTCCTGTGTCACCGCTTAAATTTTCTAGGGTCATCAGTAAACTCAGTGGAAATAA
ATTAGTTCATGAATATAATTGACCCTTAAATTCTGTCACTGTGCAAGTAATCGGTGGGTCTGCTGGATATGGCTTTC
GAGCAGACAGGTCAACTTCTTCAAACAGAGAAGAAGCATAGCATAAATTGAAGACAAATAACAAACTACTTGTTTCC
TCCTTCTTTGGCATCACCCTATGGATGGAGTATGCATTTATAATTTAACACAATCAAGAGATCTTTATTATCCTACT
TTTGGGTACAACTGCTTCGTTTCTCTTTTGAATCTCTACAGCTATTTAAAAATCTGTTTTGTAAAATTCTTTAAAAA
ACTAAAACATCAGATTCATATTTCAGGTATCTTACTATCTTATACCAACTTAAGCATCCAGTATTATCACCCACCCT
TCCCCTGAGTGAATCCTTAGCACTGGGCTCTTCCTGTTTTATCCCTGTGCATGCTGAGCTCTTTCTGGCCTTCAAGT
CTACTTCCGTTGCAACTGTTGTCTGAATGGTCTCTCTATGTCCTTCTTACTCTCTAAATATTTCGGAATTTAAAGCC
TGGAATAATCTACCTTAGTCCAAAAGATATGCTACACTATTCTAGTTCACAATGATCTCACACTGCCGTTGATACAC
AACATTTAATATCAACTTAATATCTATTTCAGTTCATTACGAGGTCACTTATGCTACATCTTATATTGTTGCCTTGG
ACTTTTATTATCTCTTCATATATGTGTTTATGGTGCTCCCACCCTCACGAGAAGTTGCAAATACCATGTTAGCTGTC
TGATGGCTTTCTATGTTGTCAGGTATACCATTTCCCAACCAGTTGGCATTCAATGATTAAGTTCATTAACAAAGAAT
TGTATGTGTTGAAAAAGATGTTTTTTTCTTAATGAAGCACTTGTTTTTATTTTTTTAATGAAATCCACCCTCTTAAT
AAATTTTAAGTGCACAATACAGTATTGTTAAATATAAGCAAAATGTTGCATAGCAGATCTTTATAATTTTTTTAACC
CTACATGCCTGATAGTCTATACCCATTGCACAGCATCTCACCATTTCTTCCCTCCTCCAGCCCTTAGCAACCACCAT
TGTACTTTCTGTTTCTATAATTTTGACTACTTTAGATACCTCATGTAAGTGGATGCGTGCAGTATTTGTCCTTTTAC
GACTTGCTTATTTTATTTAGCAAAATGGCTACAAGATTCATCCACATTGTAGCATATGGTAAGATTTCCTTTTTGTG
GCAGAATGATATTCCATTGTATGTATATAACATAGCTTTATACATTCCCCTGTCAATAGACATTTAGTTTGTTCACA
CCTCTTGGCTACTGTAAAAATGCTACAATAAACATGGGAATGCAGATATCTCTTCAAGATCCTAAATTGAATTCGTT
TAGATAAATATCCAGATGCGGGATTGCTAGATCTTATGGTAGTTATATTTTTTATTTTTTTGAGGAAACTCCATATT
GTTTTCCACAAAAGCTGCACAATTTTATATTTCCACCAGCAGTCTACATCTCCAATTTTCCTACACCTTCACCAACA
CATGTAATGATCTTGGGCTTTTTTTTTTTTTTTTTTTAATAATGGTTATCCTAATCCGTGAGGTAGTATATCATTGT
GGATTTGATTTGCATTTCCCTGGTAGTTAGTGATGTTGAACATCTTTTCATATAACTGTTGGTCATTTTAATGTCTT
CTTTGGAGAAATATCTATTCAATTCCTTTGTTCACTTTAAAAATTGGGTTGTTCGAATTTTTGTTGTTGTTGTTATT
ACGTTCCTCATGTATTTTAGATATTGACACCTTATCAGATATATGGTTTGCAAACCTTTTCTCTCATTCTATAGGTT
GCTTTTAATTCTGTTGATTGTTTCCCTTGCTTTGTAGAAGCTTTTTAGTTTGATATATTTCTGCTTATCTAGTTTTG
TTTTTGTTGGCTGTCCTTTTAGCGTCATATCCAAAAAAAATTATTGTGAAGACCAATGTCAGGAAATTTTTCCCTTA
TGTTTTCTTCTATGAGTTTCATAGTTTCAGATCTTATTTTTAAGTCTTTACTCCATTTCATTTTGAGTTGATTTTTA
TGTATAGTTTAAGTTAAAGGTCCAATTCCATTCTTTGCAATGTGTATATCCAGTTTTCCCAGCACCATTGGTTGAAG
AGGATATCCTTTCCCAGTTGTGTATTCTTGGCACCCCTATTGAAGGTGATGCTAGGTTTATTTCTGGGATCTCTATT
CTGTTCCATTGGTCTATATGTCTGCCTTTATGACACTATCGTGCGCTCTTGACTGAGGTAGCTTTGGTAATTCATTT
TGAAACTAGCAAGTGTGATGCCTCCAGTTTATTCTTCTTCCTCAAGACTGTTTTGGCTATTTGGAGTCGTTTGTGGT
TTCATATGAATTTTAGGAAATTTACCTTATTTCTGTAAAAAATGCGATTGGGATTATGATAGGAATTACACTGTATC
TGTAGATGGTTTGGATATATAGACTTTTAAATGACACATCAGATGTATTTCCATTTATTTTTGTCATCTTCAATTTC
TTTCAACAATATTTCATAGCTTTCAGCACACACATCTTTTACCTTCTTGGTTGGGTATTTACTAAGTTATTTATTCT
TTTTATTGCTATTGTAAATGAGATTGTTTTCTAAATTTCCTGTTTTTATGTTGCTAGCGTATAGAAACGCAACTGTT
GAATGATGACTTTGTATCCTGCAACTTTGCTGAATTTGTTTATTGGTTCTAACCATGTCTCTGTGTGGCGTCACTCT
TAAGATTTTCTACGTATCAGATCATCTAATTTGCAAACAGATATAATTTTACATCTTCCTTTCCAAATTTGATGTAT
TTTATTTCTCTTTCTTATCTAATTGTTCTGGCTAGTACTTCTGGTACGATTTTGAAAAGAAGTGGCAAAAGTGTGCA
TTCTTGTCTTGTTTCTGATCTTAAGGGAAAAGATTTTCAGTCTTTTGCCATTAAATGTGATATTCACTGTGGGTTTT
TCATATACGGTTTTTATTATGTTGCGGTAATTTCGTTCTATTCCTAGTTTGTTGTGTGTTTTTATCATGAAAGTGTT
GAAACTTGTTAAGCGCTTTTTCTGCAGCTATTGAGATGACCATAGATTTTTAGCCTTTGTTCTGTTAATGTTGTGTA
TCACACTGATTAGTTTTCATAAATTGAACCATTTTTGCATTCCAAGAATAAATCCTATATGGCTCTCGTGTATAATC
CTTTCAATATACTGTTGAGTTCAGTTTGCTAGTATTTTAATGAGTTATTTTGCTTCTATATTTATCAGCGGTATTGT
TCTGTACTTTTCTCCTAGTGTCTTTTATTGACTTTGATATCAGGATACTGATGCCCCTTGTAGAATGAGCTTGGAAG
TGTTCTCTTCTCTTTAATTTTTCTGAAGAATTTGAGAAGGATTGGTGTTAATTCTTCTTTAACTGTTCATTAGATTT
CACCAGTGATGACATTTGGTCCTGGGCTTTTCTTTGTTGGAAGGTTTTGGACTACTGATTCAATCTCCTTACTAGTT
TCGGCCTACTCAGATTTTCTATTTCTTCAAGATTCAATATTGGTAGATTGCATGTTTCAAGGAATTTGTTCATTTTT
TTCTAGGTTAACATACAGTTGTTTACAGCAGTGTCTTATAATCATTTGCATTCTTTTTGGATACCAGTTGTAATGTC
TCCTCTTTCATTTCTGATTTTACTTATTTGAATTTTCCTTTTTTTTTTTTTTTTTTTACTTAATCTACCTAAAGATT
TGTCAATTTTATTGATTTGTTTTTAAAAAAACTCTTAGCTTTGTTGATTTTTCTATTGTTTTCTATTTCAATTTTGG
CTTTTTTCTGATCTAATCTTAATATTTCCTTCCCTCTGCTAACTTTGGGCTTAGTTTGTCCTTCTTTTTCTAAGTCT
TTGAGGAAGAAAATGGCAAGGACATGACTTTCTTTAGCAGTTGGAAGGACAATGCTGTAAATACTCAAAAATTAATT
ATTTTTATAGTGACAAAAACAAAATAAAAAACACTTCAAAGCAAATGAAAGTTTATCATTTAATTTATCAAATCACT
AAGCAGACTGCTTGATCAGAGAGAAGATACTCATATGATCACATAAAACTGAAAGATTAAGAGGTAAGGACATTCAT
GTTATCATTACATCTAACTTTCTTATTTCCAAGATGGAGAAACTGAGGGTTGGAGAAAAAGAAAGATTTCTTTGTTA
GATACAAACAGACAGGACTAAACTCAGTATAGCAGCCTCCTAAATTCCAAAGTATCATGATACTGTGATTTTATGCA
TTCTTCAGAAAAATAGTAGAGCCACTGGATTCTGGCAAAGAAGTTATATAAAATGTCAAGTTCTTCCTTTGCCTCAG
AAATGAAGTTTTATGTTCCAAAATTGATTGGGAAGTTCTCCTTATACCTCACATCACGTCTACTATTTTACATTGTT
TACTTTTGAAGAATTTTTTTAATTGACAAATAATAATTGTACATATTCATGGAGAACCTAGTGATGTTTTTATATAT
GTAATGTATAGTGATCAGATCAGGGTAATTAGCATATCCATTATCTCAAACATTGGTCATTTATTTGTGTTGGGAAC
ATTCAACGTTCTCCTTCTAGCCATTTGAAACTTCTATATTATTGCTAACTATAGTCACCATTCAGTCGTATAGAGCA
CTAGAACTTATTTCTCCTATCTAGCTATAATTTATTTTTAAATATGCTTTTTGAATCTGTTACTATAAATTGAATGT
CACATCGTTTTGAAAATATTCTTAATTTATGCTCAACAGGCAAGATTACACACCTGTGATAATATCTTTAATTTAAA
ACATTACTCTGTTTAATTTACCAGAATATGGAACCCTAGTCATTTTAGAGGTGGAGCAAATTTCAGTGATAATCTAG
TGCAAATTTCTCATCTTATGAATGAGGAGATTGAGTCTGATATAAGGGACGAGATTTTCGTCAATGAGCAGCTTGTT
AACATTAGCTCTGTGATAGAACACAGGCACTTGTCCTCCCAGGCCGGTGTTTCTTCTACTCTATGATGGGCTGTTTT
GTTGTAGTTTTTAAACAGCAGCATTTTCACCATGCATAGTTTTCTTCCAAAGTTCGTTCTTAACGTTTTTGCACAGA
ATAACTAGATTTTGGAAGTAGAAAAAGGAAATTCTCTTTGCATCCTTGTATCTCTGGTTATTTTCTTTGTCCTTTGA
TCTCTCTCTCCTCCCCTCCCCTCCCCTCCCCTCCCCTTCCCTTCCCTCCCCTCTCCTTCCCTTCCCTTCCCTTCCCT
CCCCTCTCTCACACATTAGAGAAAGAGTTAAGGTATTAAAGAATACATAATACTATTAAATTTCCTTCACATAGAGA
AAGGAATGAAAAAAAGTGAAAAATGGTCCTCACCAAATGTCCAAACTTCTGTAGGTCATTTCCATAGTATCAGCAAT
GTCCTGTATGGTGCCTCGGGGATATGTAAGCAAATGAGCAAGTGGTTAGCTAATTCTAGCTTTGGCAAACACTTGTT
ATGGCTTACTTGAGGAGAAGTCACTTCTCCAAAGTGAAAATAATGTGCACAGGTCAATTAGAATTTTTTTGTAGAAA
AGGAAAATACTTTGTAGGGACATGGATGAATCTGGAAACCATCGTTCTCAGCAAACTATTGCAAGGACAAAAAACCA
AACACCGCATGTTCTCACTCATAGGTGGGAATTGAACAATGAGAACACATGGACACAGGAAGGGGAACATCACACAC
CGGGGCCTGTTGTGGGGTGGGGGGAGGGTGGAGGGATAGCATTAGGAGATATACTTAATGCTAAATGACCAGTTAAT
GGGTGCAGGACACCAACATGGCACATGTATACATATGTAACAAACCTGCACGTTGTGCACATGTACCCTAAAACTTA
AAGTATAATAAAAAAAAAAAAGAAGAAAATACCTCCTTATGCTCCTGACTTATTTTCTTTTTGGTTCCTCAGTCCTC
TTCTCTCTCTCTCTCTCTCTCTCTCTCTCTCTCTCTCTCTCTCTCTCTCACACACACACACACACATACCCCACATA
TACAATATGATTAAGGATATATGTGAATAATGAAAGCTTCTTGTGTATAGATTTAGAAGTCTAATGGACAAAATCAA
TATTTTCCTATGTGCATTTAATTCCCCCCTTTGATTTAGGTATATAGTCTTTTTTTAAAAAAGAGAAAAAAAATTAG
GTGACCTTAAGGTATAGATCCTACTTTCAAAAGGTTTACAGAACTAGGGAGAGGAACATGGACAAGATTTAAAGAAC
TATTTTAAGCAGAATAAAATGTGATTTATGAACAAAGCATATATTATTTGTGCGTATGTGTGTGTGCCAACAAAGAT
GCAATTAGGAGATTGCACAGGGAGATGTCATTAGAACCAACCTTAACGGGTGAGAAGTCTTTGAAGACATTTAGAAC
ATGGAAGATCTCTGACAGAGGGAACAAAGGCATAGTGACAAAAGTCAAGGGCATATTTAGGACTGGAGAGTGGTATG
TGTGGCTTGAGAGTGGGCGAGAAAAAACAACAATGCCTCTGTAATAGGAAAGTAGACAGAGGCATGACATTAAGAGC
TTTGCCAGCTGTGCTAAAAGTAGTGAACAAGAGCTAACAAAGTGAAGAAATGTACCTTTTCTGATGTGTATCATTCC
CTTATTCATATACTTCTTGAGGGGGAAATTCATTCTGTGTTGATCTAGTAAACTACTACAGGACCAAATGATAAAAA
GAAGTATAGGAAAGAATGTTTCAGCATACTTTACGAGATAACTTCCTTGTAGCTATTCTCCATAGTATTTTGAGCAT
CACAAAGCAATGAGCTGAAACTGTCTAAGCCAAAATTGACTTGTCATCTGTTAGGGATGCTTAGATGAGAATTCTAC
ATTTGAGAGCTTCTTAGATTCATTGACCACTATGTCCCATTCTAAGATCCATGAATGCGTGACCTAACTATTACACC
TTCTTTTAGTCTGATTGTCAATTTTGTATTTTCAATTGTGCAAGTTTCTAAAACTATTTTAGGAAGATAAATCTAGC
AGTGGTGTGGGAATAGACAAGAGAGAAGGGGAAAGACTCTTCAGGAAACTAAACTCACAATTTATGAGTATTCTTTA
TTGCCCAAGTCTTCCCAAAGTCTTTCATCAAGAAAGAGGCATTGCAACTCTCCTTTTATAGTTTGTTTTTATTCTGG
AGCAGTGATGTTTTGGTGGAGTTGTTCCTCAGTGCGTAATTAAAGGGCCTATGACAATTACAGTTCATCTCCTGCTG
CTCAAGGTACTGCAGATATTTGGATCTACTACTCTCATTCATTTCCAATTAATGTCAGCTTTAGATTTCCTTCAGTA
TGCTATGTTATAAAATTTGATTATCGTTGTGCCCACCTTCCCACTTAATTTCAAGCAGGTTTCTCGATTACCTGACT
AAACTAATGAAATCTGACTAACCCAATATCTGTGGACAGTAGTGTGATGTTACTGATTTTTGTATGATTAGTCAAGT
CATATTCATGCCACGTTTTCATATAGTACCATAAAGGATATTCTTCTCGTGGTCCTTTTCTTTTATTCTGAACATAC
AATGAGAAGACCGGTAAAGTGGGCTAGGAAATTAAAGAAAAATACAAATGGCAAAAAATATGGGTCACTCGAAGTCT
AGAATAGAGAGCACAATCAATTTTGAATTAAGGGGTGATAAGGTGATTTGGTCAGGTGACTGGTGAAACAGGAAAGA
AACTATACTTTTTGAAGTGTTTCATCCATGTGTTAAGATTCATTTGGGGTCAAGAATCTAAATTTCATATCCCTGGG
AGTGGAAACTAAGTAAAAAAAAAAATTATGGACCTTGGTTTAATAGCTAGAGGAGCAAGAGTGTATCTTTATGTGAC
TTAACTTCTATGTGAAAAGTGAACCTTAAGATTAATTATTGGGGGAATTTACTTACTCAGGTTCTATGCCTAGATGG
TCTGCCCAACTAAGAAAACTTATTTTCCTGTTACTCCATCCTATTTTTCATACTTTTATACTGCACTTGCAGAAAAG
CATATATTTCTACCCAATACGAAAATTCCTGGGAACATATTTTTCTACATTTCCCAAATTACTTCAAAAAGTAAACT
TAGGTTATTTCATGATCTCCATTACAATGGACAGGTGGCCTTATTGAATGTTGTCCTGTGAATACAAAGATCCAGAG
TTTAAAGAACAAGGTGTACTTGCATCTCCCACTTAGGGTTTGCTTGTGGTGGAGAGAGAATCTAGTTTGCTTAAAAG
GATGACAGTGCAGTGCCCCAAAATATCTGATATCATTAAAAGTCTCATATTTGTCTTTCGTAACTTCTCTAGGGCTG
TCGATGACAGGAGACCCTTAACTCCTATGCCTTGATTATGTGAATAAGCACATGAAAATATTTTAGTTATCTTAGTT
CACTTTTAAACTAAGTTTCAATTATCACTAGATTCTAAATATCATCATTGAGCCGTTCTTAAGGAACTGATTTTCTA
CATATTCATTCACTTCACCTATATCTAGTGTGTCTACTATTTGCCAAGAAAAATTTACTCTCTTAATTCAGCATTCC
ATATACTTAACATCATAAAAAGTAGGCCATTTTTAGTTTTCTAAATTATTTATTTAAACATTTCTTTAAAATTACAT
TCTATCATTACACTATATTTCAACACTACAGTAAGCAGCCTATTTTGTGATTTTTCCTTATATAAAATACATAATTG
AAATTAAAAATGAAGTTACCAAGAGCCATTTTCACTCTGGGGAATGCACATTTATAAATTATGGGGTTATTTTTTCT
TCATCAGCTTTCATATTATTAAACTTTGTCTCTTCATAATTACAGAGATGACTAGACACAGAAGGGAATTTAACATT
TGGTGTGCATTTGTCTAACCTATACTTTATGTTAGAAAATACATTTCCATTTGAAAAAAAATCAGTAATTGTGGGTG
TGATCAAGAGGGCAGCCTGAAAGTCGGGTGATGTGACTCACACCTGTAATCCCAGCATTTTTGGAGGCCAAGGTGGG
ATTATCGATTGAGCCCAGGAGTTCAAAACCAGCCTGGGCAACACAGTGAGAGCCTGTCTCTATTAGGGGGAAAAAAA
AAAAAAGAGGAAGTTAGCCTGAGGCAATGTAAATGAAATACATATTTCAAGGATATTTATACATGATTCACGTTATT
CATATAAAGATGTGCCAGAGAAGACTATAGGTACGTTATTTTACACTATTTTGCTAGGATTTTAAGAAATTCAATGT
GTTTTTATTTCAGTTAACTTAGAAAACTTACCTAACTTATACTTCTCATGGACACAAAAGTTTTTAAAGATAGGATC
AAAAAGCCCACATGGTGAAGCATTTTGAACTGGATGAAAAACATCTATTATCTTTAAAATTTTATGATATTACTGAT
TGTAATAGACTCCCTTTTTAAGAAATCATTCCTTATAGAACATAAGGTTTACATTTACAATCAACAATTTCTATCCT
TACTACAATAAAGGCACATATAAAAAGTACAGTTGCATATTTAGCAGGTTTAATTGTACATTTTAATGTAGAAATCA
ATTCAATTCTTTCATTTATCAGCATTATTACAGTGATTTCAAATTAAGCATAGGTAACTTTGATATAGATAAATGAT
GTACACAGCAGTTAAATTTTATTTTCAATTATGTAGTAATTGTATAACCTAGGCAGTATAATTTGTAAACTTTGTAT
TTTATTATTATGCTTCTCCCACTTGGCATAAGCACAACACTTCCTAAAAGCATAATTTTCTATAGACTTAATAACTC
CCTAAAAACCTGTTTTGGACCCCTATACTATTTGATATAGGCAGAAAAAAAACATAATCCATGCTCAAATTTGAAAA
ATGACTGGTCACATTTGGTATAATACTAAAGGTAAATAAAATCAAGAGTCTATGAACATTTCCGGACCTGCACATTT
GTTTTATTAAAATGCATAATTGTCTTTAGTGTGTTTCTATTTGTTTATACTCTACTGATTTTAATTAAAAATACCAA
AATACGTTTATTAAAAAACTGTCAGAATCTAAGTTGTTAAATATACTTAACTAGGAAAGTAACTGTTTAAACGAGAT
AATTTATAGAGAAATGTGGTGTATTGCCAATTAGATGTCAAGATACAATACAACTGATAATGAAAAAGTAGCATTTT
CTTAGGGATGGAATACAGTGTAAGGAACACCCCAGTAAGAATACAAAAATTACTGAAAAAAAATCTTCCTTCCTGAA
AAACCAAGTGCCCTTCAAGTGCAGAACCTCATCCAACTAATTGTTAGGTATCACTAAAGCCTGATACCTTCAATTTT
CTGGATCATTCAAGCTGTATTTTTGAGTCCTTATACTAGAGGAGGTAAAGAGCTATAAAAACACTTAATGGTATCTG
ATGTGAACTGTGGATCACTTTGACCCATCACTTCTACGTCTACATCTTGGATAAATTCCCATTGTTGTCATAGATTG
TACAGGTTTAATGGTGCGTTTGTGGAGGGGGCTCGCTTATAGAAAATGGAGACTCTGAAGGGATAAGGAATAAATGT
ATCACTTCAGGTCTTTTATTTGAAATTGGGGTCCAGAGAGCCTTTTTGTATCAGACTTGTCAAACCATTTCCATTTA
GTAATTATATATGCACTAGCACTTATTCCTACTTACCTCACCTCTTTATGCCCATTTCCTTGTAGTTGCGGTTATGC
ATGAATAATTTATTGCACCCCTTACCAACAATGGAATAAAACTTCCATTCTGAAAGCTTTCCATACTCATTTCCAAT
AGCAATAGGGTTTTTTTAACGGACGTATTACAAATGTACGAGTCAGTTGAACATAGTATTCCTCTTTGTAAGAACTC
CAAGTGGATGCATGCTGTTGTCTCAAATCTCAATTAGACCTTGCTTTGAGGTCCCTTCATTGCCAGTCATCTGTTCT
CCTTCCCCTGACTTGAGTATTTCTCCAGATATAGATAATACATTTTCCCAACTCTGTGTTCCAAGAACTGACAGTGG
CTTTCATTCATTTTGTTTGTTTGTTTGTTTCTTCTCGTTCTCAAGTATCCCGCAGTCTACTGTTTCTTCCCTCCATT
CGTTTGTCCTTTCAGAGTTTCAAAATCCAGCATAGGTACTTCTTCTAAAATGTCTTACCCTTCACATACACACACCA
CTTGAGACCCCATCAGCCTCTGTCCACACAGTTTGGTTACATTCATAGACTATTTTTATACATCAAAATATTTGAAA
ATTTTAGGGTAAATCTCAGTAGTCATTCATTTTTGCTCTTATTCAACCAATACTAGTCAATCAGCCTGTGCCAGGTT
TTGTTGCAGGTACCAGGTATCCATCCATAAAGAAAACAACGTCCCTTTGTTGTGGAATTTACATTTTAGCAGGGGAG
GCAAAGAACCCAATAAATATGATAAAATATCAGATTAAAAGTACGATGAAAAAAATCATCAGGGTAAAGGAAAAAGG
GAAGCAGTATTTTAGCAAGAGTGGTGAAGAGAGGAGGCTGAGAGTGTGACATCTGAGCAGAGACCTAAATCAAGTCA
AGGAATGAAACATGCTACTATCTAAAGAAATGAGTCAGGATAAGGAACTAGTAAGAGCCGAGGCCCAGAGATGTGAA
TATGCTGTTCCAGGAACAGCAAAGAGACTGGTTGATATGATGTGAAAAATGAGAAGAAACCTTATGATATGTGTCAA
GAGAAAAAAAAAATTTAAAAGCATGCTTGGGAACGGAGGCCTCCAGATGAAAAAAAAAAACACAGTTCAAATCCTTG
TTCATGCATTTAGTTTGCTTTGCAATCTTGGGCAAAATGTTAAATTTCTGTACGTTTTATCTTCCTCATTTTTAAAA
TAGGCACAAGGACATCTACTTAATAGGTTCATTGTGAGGAGTAAATGAGATGATATATCTAGGATGCCTGGCATTAT
ATCATACACTTAATAATACACTGAATAAATAATAGTTATGTCTATTTATTTCCTTATCGTTTTTATTATTATTTCAA
TGCACAGACCTGTTCATAAGATAATGATAAATATTAGTGGCAGAAACTGAAGATGTTATAAATTATTAGGAGGCGGG
ACCACTCAGTTCAATGTATCTGTTTTAATATAGTCAGCAAAAGTGTGAAGATACCAACAATTAAATTTCAATGCATT
CTTCCATTTCACTAGTTTTATAAACTGATGAACTACCAGAATGTCAATGTATGAATTGCATACTCATTCTTAACAAA
CAGATTTGCAAAATTATGTGTAAAATTAGCCCTCAGCCTTCCAATTTGTTATTGTCATATTTCATGGAAATACATAA
TCTGTAAATTTTTGTTTTAATGATATGTGAAACTGCCTAAAGTAGAGTCTTGGCAACTACTTCACATTTGTCCTCCA
GAGATAGTGGATAAAAGTGTCAATAAATGAACACTCTATATTCACTAATCACAGGCAAGGGACAAGGAACAGAGTGG
TCACAAAATACCACAAAATTAAAGCACATTCCAAATTAAATATATATGTTTTTATTACAGATAATGTTTGCTAGACT
CTTTCTAATTATCTGCAAAGATTTTAGGAATGTTTTAATGTTTTAATATTTACACACCTGTGTATTTCAAGTTCAGT
CAAACACTATTGTTAAAACTAAATCTTCTCATCTCTAATAATAAGATGTGAACTTATCTTGGAAGGTGGTTATTAGG
ATGGGAGAGATAATGTATTTCATTCAAAGTAAAAATATTTCTCTGTTTCTATCTTTCTCTTTCTCTGTCATCTATTT
ATCATCTATATCCAGGTATCTATGCACCTATGTAGACTAGCATTCAATGAACCATAGATATTATTAGTAGTAGAATT
GTTACTAATATTAAAATAAGAAGTATTTAAGAAGAAACATGTCCTAAAGCATAAGGTCAATTATTACTCTCATGTTT
TTTGGCATATGAAGCCTAAAAAGTGTCAATTTCAAGAGAGTATTAATAAAGATTGTGATAACTGAAAGGTTCCTGCT
TGAAATTTTGTGTGGTCTTACAAATATATAAACTCTAAGCATTTCAGTGAGCCAATTACTGACTAGGCACTATGTCT
TATGACTCTTTTGTCATAGTATGTAAAAAACAAAGAGTAGAGACATCATAAAAATTATAGTAGATGGGCACTAGGGA
ATTACGCAAAATAATTTGTAGATTTAATGTGAAACCAAAACATCTGTTCAAGTCAATTTCCCACAGGTCATGTGGCA
AAGAGTATGAGTTCCAGACTGAGGAGAGGAAAAGGTTGTTCTTCCACAGGGAAATAAACTGAGTGTAATAAACATAA
TTTTTCTTCTTAAGCATTATTTAAAACAAAAAAAATGCCATTAAATCTATCTTTCCTGCCTCTCTTATCAATGCTCC
CTTCCCTTTCACCACTTGTTTCAAACTCCAAGCCTTGGGATTTTATTTTGGCTTTTTGCCTTAATGTAACTAAAATG
AGAGCATCACAAATATGAAGCTCATCAAATAATTTAGCAGCATTTTCCCCTGTTTTTAACTTTCTCTTTGGAAACGT
AGATTTCGAAATTTAAGGGCCCAAAATATGAAATGCAATTATAATAGGCCATTTGTTCATTCAGCTTGATAAACTTG
AATAAATAGTATTGAACTTTTAATGCAAAAAGAACAAAACAAAATAGAACTCTCCACGAAGAAACTTTTCAATGTTT
GCATTTCTGTGTGAGGAGAAGGGTAATGAATGTGGGAACCTTAATGGAATCCATGTTCTTCCAGTGATGACAAGGGT
CAAAATGGAGAAAAATGGTCACTTTCTACCCAGTACATTATATTAGTTCTATGTGGACAACTATAACATAGCTGATG
CTGGTTTTCAGGCCATAAATGTAGGTATGTATTTTCCTACTATTTATAAGGCAAAATTTCTATTTGTTTAATGATTT
CTATATAGGTAGATTATTCTGTCTTTAGGATTAAAAACGACCTGTAGACCAAGAGACTTTCTAATGTCCACCTTAGA
GTATATGGCTTTTACTGTTACAGTTTCCATTTCCTTTGCTTGCCCCTTTGAGAGAAGGAAAGGAGACATTTGGGATA
CATACATCAATGAGGAGCTATTAATGAATAAATGAATGAAATTGTCAGTCAATTTATCCACATGATCATCAATTGCC
AATAATTTTATCACCTCTGTGGGATTAAGTAGAGGTAACAGTTTAGAAATTTGATTTTTTGAAAGCATTTAAAATGT
TCAAATATATCACTCTGGTAACTAAGGGAAAGTGTATTATTTTCTTATGCTTAGTCTTATTTTGGTTTTGCCTTTTT
AATTTAAATTGAACACTTATATCAAAGAGCTTGCAGGATTATAATTTGAATTTTTGAAGCAAAGATCATTTTCTTAA
CATCAAACAAAGAGTAGATACAATAGGAATAAAATCGGCAGAAAAACAAGAGTATCAAGGACAGACGGGGAGGGTGG
GTCTGTGTTAGCATGTATTGCTATGAAGAAATAGCCGAGACTGGGTAATGTATTTTTAAAAAGAGCTTTAATCGATT
CATGATTCTGCAGGTTGTACAGGAAGCAGGACACCAGCATCTACTCAGCTTCTGGGGAGGCCTCCGGGAGCTTTTAC
TCATAGTGGAAGATGAAACAGGAGTAAGCATGTCACATGGCCAGAGCAGAAGCCAGGGGGAGGTTGCCACACATTTA
AAAAAAAAAAAAACAAAACAGATCGCTCAAGAACTCAGCTGCTATCATGAGGACAGCATCAAGCTGTGAGGGATCCA
CCTCCGTGACTCAAACATCTCACACCAGGCCCCAAGTCCAACACTTGGCATTATATTTCAACAAGAAAAAAAGTTTA
ATTGGCTGATGGTTCTGCAGGCTGTACAGGAAGTGTGGCACAGGCATTTGCTTGGCTCCTGGGGAGGCCTCAGGGAG
TTTTTGCTCATGGCAGAAGGTGATGCCCACACACTTTAAAAAAAAACCAGATCTCATGAAAACTCACTCACTACACT
GAGGACAATACAAAACCATGAGGGATCTGTCCCCATGACCCAAAAACCTCCCGCCAGGCCCCACCACCAACATTGGG
AATTATATTTCCACTTGAGATTTGAGTGGCGGCAAATATCCAAACTATATCAGGGCTCATGTCCAGTTATATGTCAA
CATGCCTGCATTCGAAACATCCTGTCCAAATCACTGCCTTGTCATAATACTTATATTTTTCTTTATTGAATACGAAC
ACAAGAAGATTAAATAATAGCATTTCTACTTTAAAACAGTGGGCACCATATTAACATTGGAATAATAGTAGTAATAA
CGATAGTAATAACAATGATATAGGCTGGGTGCGGAGGCTCACGCCTGTAATCCCAGCACTTTGGGAGGCCAAGGCGG
GCGGATCATGATGTCAGGAGATCGAGACCATCCTGGCTAACACAGTGAAACCCCGTCTCTACTAAAAATACAAAAAA
ATTAGCTGGGCATGGTGGCAGGCACCTGTAGTCTCAGCTACTTGGGAGGCTGAGGCAGGAGAATGGCGTGAACCTGG
GATGCAGAGCTTGCAGTGAGCCGAGATCGTGCCACTGCACTCCAACCTGGGCGACAGAGCGAGACTTCATCTCAAAA
AAAAAATTAAATAAATAAATAAATAAATAATAACGATAAAAGGATATGTGTAGGTTTTTTTTTTAATAGGCTGTTAA
CATTAATAGGCATTGTGATTTCAGGGATATCATCAAACATCCTGGTCCTAAGACATCCCCTATTGAATAGGAAGGGC
TTAAGTTAAACTTCTCATGAGCCACAATTTTCTGATTATATGTTTGGTGTGTGTAATAGCCACCTCAGTGATGATTT
GATTAGCCTGGACCCTTACATAATCATTGAAGTATACCCATGTTCCTTTATATACTTCTTTAGTGTTGAAAGCTCAA
AATTAAGCAAAATAGTCCCCTTGATAATGTTTAGATTCTTAACATTTGCTTTCTAAAGCTGGCAAATACTCTCTTCC
CAGTGTCATGAAGTTAAATAACATGTTGCTTAGTGAGGACTTTAATGTTGCCATGCCATAGGAAGACCTTATTCGAA
ATCCCCTTACCTGGGAGAATGTCAGATTATTACCCCCCAACTTGTTTAACACTTTTAGGATTTTAAAGGTGTTCACA
TTTGTATTAGAACAAAATACTATTGAGAAACATTTCTAGAAAAAAATTATCTTTCCAAATTAAAATCAGTGGTATGT
AATGTAGGAGTCTGATTATAATGATTAAAATACATGGGCTTTGGGCATACTGCCTAGGTGAAACTCCTGGTTTATTG
CATCACTATTAGTATAACCTATGGGAGTTAACCTACGTAAGCCTCAGTTAATTTTTCTCTCAAATTGATCTAATAAT
CGTCTCTCATAGGCTTGTTTTGATAGATATTTCAGTGTATATAATATACTTAGGACAGTGCCTGATATCAGTAAGTC
TCCTTATATGCTATTTTTCTTTCTATTTTAATTATTTATGCAAGAGAAACTATTATGCTTTAACTCAATTAAAATAA
AATGCCTTTGTATTTATTCATGTCAAAGGAAATATGCAAGTATTGCATTCACTTCCTAGGTGCCTTTTTGAATTGAG
CTTTGCATGGTTAGTTTGTATAAAAGGTTCAGTGAACTTTCTCATAATGATTTTTTATTGAACATATGGAATCCATT
AAGTGTTAGCAAAAGTCACTATCCACTGAGCTGTGTCCAGGGGCTGACAGTTATGTCTATCTCTTGCAAAAATAAAC
ACATACATAAATGCACTAAGACGTATATTACCTGTCGTCATCTCTTAGAGCATTTCCATTTTTCTTTTAAGTTTTTT
CTTTCAATGGGTTTTTTATCTTTGTGAGTACATGGTAGGTGTATATGTCAACGGGGTACATGAGGAAGGTGTATATA
TTGATGGGGTACAAGAGAGGTTTTAACACAAGCATTCAATATGAAATAGTCACATCATGGAGAATGGGTTATCTATC
CCTTCAAGCATTTGTGCTTTGTATTACAAACATTCTAATTATACTCTGTTAGTTATTTTAAAATGTACCATTAAGTT
ATTACTGACTATAGCAACCCTATTGTGCTATGAAACAGTAGATCTTATTCTTATTTTTCTAACATCTTAGAACATTT
CCACAAACACTACCTGCTTGTTAAATATACCTATTCTAATCTTCATATAATCAATTACTTTTTTCCTCTAGAATGTA
CTATGACACATCCATGGGGAAAATGTAGTAATCTAATTAAGACTATTTCCTCTCATTTTATATTTAAAAGAATGTGC
TCTATCAATTTATTTACTTGTACAGCCGTAGGCAACCTCTAAAATATTTAAAGTTCTTAAAAGTCAGATATTTCAGT
TAATATTGTGATTATATAGTTGATTTTGATGAACATGTTCATCTACCAGAAATAAATTATACACACACATTGATATG
GTTAGGCTTTCTGTCCCCACTCAAATCTCATTTTGAATTATAATCCCCGTGTGTCAAGGGAGAGACCAGGTGGAGGC
AATTGGATCTTGAGGGTGGTTTTGCCCATGCTGTTCTCCTGATAGTGAATCATGAGATCAGATGGTTTTATAAAGGG
CTCTTCCCCCTTCCCTCCTCACTCATTCTCCTTCTTGCCACCTTGTAAAGGAGGTGCCTTGCTTTCTACTATGCCCT
TTCTACTATGCCCTTCACCTTCTACTATGATTGTAAGTTTCCTGAGGTCTCCCCAGCCATGCTGAACTATGAGTCAA
TTAAATCCCTTTCCTTTATAAATTACCCAGTCTCAGGCAGTTCTTTATTGCACATATATGTGTGTGTATGTGTATGT
GTGTGTGTGTGTATATGTATGTATATATGTATACATATGTGTGTATATGTATGTATATATGTATGTATATATGTATA
CATATGTGTGTATATGTATGTATATATGTATACATATGTGTGTGTGTATATATGTGTACATATATATATATATATAT
ATATATATATATATATATATATGAACAGAGAGAGAGAGAGAGAGGGAGGAAGGGAGAGAGGGAGGGAAGCATGGAGA
AAGAGAGAGTAATAGCCTAAATAGAAATAAAACTAGCTCCAAGTACAGGTTCGTCAACACTCTCCTATCATACCCCC
ACCAAAGTTAATGTTAACCACTTGGAGCCCTGTTCTTCCTTAGTTGTGGAGTACTTTAGCAAAATTTTAAATCTAAT
TATGCCTAATTCAACGACAGTGCTAATTTGAAAGTGTTAGAAACTGAAGACCTATAATAATAATGAGAGTTACAAAA
CATAAATAGTGAGACAATGATGAATGTAGTGGATGCATGTACGAGGGCTATCATTTGACAGTAGAGATGATGCTCAA
GGACAGACAATGAGTCTTTCAATGTGTGGAGAATGTGCTGCTGTTACAGTGATGTACAGGAAAGAAACAAAAACTGA
GGAAGTATCAGTAAACAAAACACTCAAACATATGAGTATACAGCTAGAATAAAAGCAACAGTACTAGATGACAATAA
GCCCAATGTTAACTCAGAAAGCAGAAGGTTTTTAAGAATTTGGGGAATACTGTGGCTGATGATACTTATGTCTCAAG
CCACAGATGCCATATGGGCTCTGCGCCCAGTTGAATCGGCACCACCTGGCAGTAAGTGGGCAGGTCCACGACTGCCA
GGACATCCCTTCCAACACTTGTGGAGATCACCAGGAAGGGGGGAGAGACCTGCCTTGACAGATTTTCAATGTGGGCG
AAACAGGTCTATTTTGAGAAAAGATGTTCAATAGAACATATGTCAGCAAGGAAGAAGAGATGATGCTTAGTTCTAAA
GCTCCAAAGAGCTGGCTTACACTCCAACTTGGGGAAAATGCATCCGGGAAATGCAAGATTAATCTCATCTTAGCCAT
TCTTTTGAATGGATGGACATGACCCCTTTCTACTTGAAGACAGAAAACATAACCATATTGATTTCAGGTTTTCTTCA
TTGGTTTCCATTTAGGATTGTTCCTCCCCATCTTCTTTCTGTGTAGGCATCCCAGTTCCCAAGTGTTCATGAAGCAC
GTATGGCCTTCAGGGGATGTGTCTGTATACATTGTTATCTTATGGATGCACGGTTTTGTCTGCACCTTGGTTCTGAA
TGTCTTTACTCTTGAGCATCTGCCCATGGGTCCCCTTCTCAAGGCCTCAATTTCTTGAGTTTAACACTGCATGGCCC
ATGCAGCTTTTCAGTTAAGCATCTCTTGCTATGACCAACTCTTTTCCTCAGTCAACTCCCACACTCTTTTCAGGGAC
AGGAAAAATGTAGCCACTTGCTGGCTGCACTCTGAGGCCTCAAGAAATTTAGTGAATCTGCCTTTGCCCTTCTTGCT
GATGAAATACTGCCACATCAGGCCCCCTCTTCGGAAACCTACAAGCATCTAATTTTCTTGCTTCCTCCCCAACTTTC
TTTTTGACTCCCCCCCATCCAGAGAGTTCTTATGTCTACTGTACTAGGAAAAACTCATTCTTAAGGTATGGTTTTCA
AATCATTCTCTGGTCTGGACTTTAGCTACGGTTTTAAATGAAGAAACAACCCAGAGCCAAAATATAATGAAACTATT
TCCTTCTTCCACAGAGTGGAAACTGCTTTGGGGTTAAAGGGCCAGTGAACCAAATAGAAAAGGATCTCAGGGAACAC
AGATTGAAGAGAGAGAAGAAAAAATATGAAGGCATTGTTGGTTCTCTTTTGAGTTTAAAATCTAGTGGGGATTGTAA
GCACACACACATATACACACACACGCTTACACACACACACCAGTGAAGTTATGAAGGATTTTGTCACTCCAACGACC
TTGAATTTGATTATCTAGGTCAGTTGTTACCAAAGTGGAATGTACATGCCCAATAATATGCGTGCTAAACAGTTGGG
GTAGTGAGAAAAAATACTTTTTATTTATCTTGTTCTCTAGAAATTAATATTTTGATTGTATATTTTATAGTGTATGT
GATGTGTAAGTTGTGTCTACAAAACTAGTGTCAATGTAATTTAAAATTACATATGTCTGTGAATATATATTTATATA
GGGTACATGCTTAAAATGTGTTTACTTCTGAGGTACATGAACATTTTTCCCCCAGGCACAGAAAGACAAATACCACA
TGATGTCACTTAAATGTGCAATGTAAGAAAAGTTGAATTCATAGAGATGTAGAGTAGAATCATGGTTAACAGAGGCT
TGGGAGGTGGAGTGAGGGAATAGAGAGTTACTGTTCAAAGATTACAAAGTTTCAACTAGACAGAGGGAATACATTTT
GAGATCTATTTCAGGAACATTTTGAGACCCTCACTCTAAGTAATAGGAAATCATTACTTTAGTTAACATATTTGAAT
ATGAGTTGTGATGTTCTATATCGTTTATTTGGATTCTACTAACCCACACCTAGATTTTTATGGCATTACCTTTTTAC
TCACTGTGAATATCCTACTCATAGACAGATGCCCTGGGAACTTGGACTTGAGGCACCCAAGAACTGAGACAGTGAGA
TTTGGGGGCACAAGGATCTATGGATAAGTTCATCTTAGTGATGATAAAATCAATTTGGCATGTTTCACGGACAGTGT
GCATTTTAGAAAGGGTAAAGACTTGGAAACGGGATATTTTTGAGCCCAAGTGTTTCCAATAAATAGCTGTATAATTT
GAAGCAAATAATTGATTTTTTGTTCTCTTTGTGCCCTCGCCTGTAAAATGGGAGAAATGTATTCCTTTCTCATCCTT
CTCATGAGGCCATTGAGAGTATCTAATGAGATCAGACTGTGACATAGCATAATAATTCTCATTTCTTGAAGGCCTAT
TATACACTTTGCAAGCACTGTATGTGTTGTTTCTACTTCTCTTGTTCGTTTTTCCTGGAATAAATATCCCCCCCTCC
TTTACATTGGATTGCCATTATTCACCCTGTAAGGAAGGCTTCATGGTTCTCATTTTCATCTGAGAAAACTTAGGCTC
AGAGAAGATCAGTAACTTATCTAAAACACACACATACACACACAGACATATCTATGCCCATTATTCTTAACCTAGTT
TCTCTATTCAGGAGTTATCTCTGCTGTCTCTGCTTCTGATTATAATCTGTGTAAGCTGATCCAAGTGACACGATTAC
AGGGAAATTGTAAGCCCTTTGAGAGCAGAGACTACCTATTGATATCTACATTTTAAAATTTGATTTTAGCCAACCTG
TTTATATGCAATGACTAACAGGTTAGTTTGACTTGCAATAAATATTCCAAATCCTAGACTAAGTAAATTTATTAATG
TAATGATTTAACTTGATTTTTTCATTGGCATGTTTCCCTGAAGTCGTCATGCAAAATTGAAAAAAAAAAAAGTATAG
TGTGTGATTCTAGATTGAAATTCAGGAATCCTCCAGGGTTACCTTGTTTGCTTTCCAAATAGTTCAGATTGCTTAGT
CTGACCAACAAGGTCCCTGACACTTGGAACTCTGTCTATCCCTCTAATTGACTTTGTCCCTGATGACCTCGCCCAGA
GATACTCTTCACCCCAGCTATACTGTGTTGCTAGAGTTTCTCTGATATCCCATGCTATTGTTTCCTTTGTTCTCTTC
ATAAGGTACCATTTCCCACCCGCCAACTCCTGTTTTCCTGATGGACTTTTGTTTCACCTTACAAGATCATTGCTAAT
GTATTTATTTTGAGAATAAAAAGTGTAGGAAAGGTCACGGGACAAAGCTGTACACCAGACCTTTCCCAGACGAACCT
AGTGTATAATCTCCCTAGTCCAACATCATGGCTTAAGGCAGTCGATAGATCCGTCTTAATGTCCCTTTTGAGTTTTC
TACTATTATTATATGAGGATTTATTTTTGTCTGAATTCCTCCCTAGATTTGCCCTAGAGAGCAATGACTATTTACAG
TTTATTCCTCTTTGTATCTCTTATGTTAAGGCCAGACCTTGGCACATATTCTAGCTGATTAGAAGACGTTTGTTGAA
TGACCAAGTGATTGAACAAATGACCATGTGCTCTGCCACAGTCCGGTCAGTTCTACTTTGGTTTGGTTATGTGTTTG
CCACATTAAAGTTGTAGCCTGGGAAGTTCAGTTGTGAGATGTCTGCAGAACATGAAAAATTGGAATAATGAGGTTAT
TTCTAAAATTGCTATAATTTAAAATAAATAGTGGTTTATTCCATATATGAATATACACTGGAAACAAAGAATTTCTA
GAATACTGGAGATTCAATGATAACATCATTGAAATTAAATAAATAATAGGATTATGCTAGTTACTTTCTAATTTACT
AGAAATTGACCGTGTGCATGGCACGTATAATGAGTATCATGGGATAGTTACAAAAAGTGGTGCTTAGTGAGTTTCTG
TGGAAAATCTCGGTACCAATAAAACGGAGGATTTCCAGAAATCGATATTCCTCAAAGCTTGACAGTATTTATGCACG
GTTACACTTTGTGTGTCTTTCGTTTGAATCAATGGAAGGAGGCTATAACTGAAAATTATTGTTTTAGTGTATTATAT
CTTTAATAATAAGAGTTTTAAGAATCTATCATTAGAAATAATTATTCCTCAATTTGTAATTCTCAACATTTGAACAA
ATAAATGCTCTGTGTCTATCAGTTAATCTTGCCCATGAAGATTTAATAAAGCACGCTAGTTTTTACAAATGTGATTT
TAGAGATGGTCATTACTTGGTAAAATATTTTGTGTTAACACTTCCATGAATATGTTCTGTGGGAATATACTGCCTCC
ACATTGCTTGCTCATGAAGACATGATTTTTCACATCATCCTATCAGTATTTTGAGAAAGAGATTGATCCCATATTCT
ATGAGCATTTGAACATTCTCTAGTATTTTTGTTTAATCATTAAAACAACCCTTGAAGTCTATGTGCTACACTGGTTA
TTTCCCTCTTGACTTTCCTTTACAGATAACCCTCTATCATAAACAACCTATCTATATTTGTTGTCTCCACATCATGT
TGCCAGCCCTGCTTTAACACACTGCACATTGACTTCTAGCAGCAAAGGCTCATGGGAGGTACTCTCATCAAGGACAC
TGATGGTCCTCATGTTGCTAAATTTGGTGGGTCCTCTACAGTCTTTATCCTAGTTCACCTTATTATGGACCACTGTC
AACTCTGTTCTGCTTAAAACACTCTGTTCCTTGCTTATATGACTCTACACTCTTAACTCCTTTGTGAATTCCTCATC
TGCCCTTCCATTAAGTATTGACGACATCCTTCATAGTTTTGATCTAGGACCTCTTTTCCTCTTACTTGACATTATGT
GGGTAATCTTGTCTTTGAACGCAATTACCATTCTTATGTTGATGACCCTTAAGCTATAATTCCAGCCCAAATCATTT
TTCTGAGGAAGCTACAAGAATACACAAATGTCTAATAGATCTCTATTTAGATGTCCCTCAGGTGCTTCAAGCTTAAA
ATACTCACCTGAGCTCATCACCTCATCTATAAATTCTGCTTCTCCTCCCTGGCTCCCTGATTTATTTAATATGACCA
CCATCCACTTAGTTGAATAAAGCAGAAGCCTGGACACCATCTATACCTCCAATTAATCACTAAGTTTTGTTGTTAAA
TACGTTCTTACATTTTCTCTCTAGAATGTCTTATTTTCCCCATCTTTACACCCAAAACCAAAAGTCAGATGACCCTG
ATCTCCTGCTTAGATTTCAAAACACTATCTCTTGCCTAGACTCTGGAATTTCAGTCTTGCTCCTCTCCAATCTATTT
CTACACCCTAGACTCTGGAATTTCAGTCTTGCTCCTCTCCAATCTATTTCTACACAAAAGCTAGAGTAATTTTTTAA
AAAACAAAAATCTGAATGTGTTCATTTTCTGCCTAAAAGCCTTCAGTAATTCTTATTTGTTCTTCCAGGGATAGAGT
AACAACTTTCAGACCTAGTTTATTAGCTAGTTCTTTAACCACAAAGGACTCTCTCACTTGTCTACTCCCCCTAACAC
ACTTCGCCCTAACCTTTGCCATTCCTCCCTTTCCCTTTTCCTTCCCAGATGGACTTAAGTCCTTTCAGATTCTTAAA
TGTTTCTTCCTCCAGTCTCTTACATCTCTTTTCCTTGTAACTCTAAAAACTACTTAGCTTACGCAAGGAAAAAGGTC
TGTACAATTCCCGGAATCAGCGATCCTAACGTTCCCTGTTGTTTTTTTCGTTGGGACATGAATTCATTCACAGTGGC
TCTAAACATCACCACCCCTGCCTATCTCTCCCATTCCTACTTTATCTGAGCTTATCCATACTCTTGAAGACTTACAT
ATTTTTTTTCTACCAGGAAATCATTACTAGCCTTATTATCCCACTGTCCAAACCAATAAGTCTGATTAGGTATCTGT
ATATATTTAATATTACTATATGTGTTTTTCTAACACTCTAGTAGAGGAGAAGGTGTATTTCTTTCTGTTTTTTAGAA
GCCTGTATTTCTGCTATTATAGCTCTTAAGGAACTCTCATGCAATTGCCTACTAGAATGTAAGTTACGGTAGGATAA
GAACTGGATCAGTCATATCACACATCCACATATAGGACCTAGCACCATATCTAACACACAGCAGGTACTCAATACAT
TTCTTTCCCAAATAACTAAAGAGTTTAAACAAACCAAAATGATTAAATGAGAAGTAACTGTTTTGGTAATTCTTGTG
TCCTTACTAGAGTCTAAATTGAGTGATTTTTATATCATCAGTTTATACTCCCCTTTCCCAACCCCAATTCTTTCTTT
TTTAAATTTTTTAAATCAAATATGCCTTAAAACTTCAGGATCAGTTGAGTAAAATGATGCTTTTGTCGTCTTTTGCA
AAATAATTGTATTTCAGAATTTTGATTTAGATATTATAAACACACCTAAAATAATAGCTTTAGTCTTAAGATGAAGT
GCTTCTTAAACTCCCTAAGATGGGTTGGACTATGGATATGAACATGGACAATATCACATTAATTTGTGTACACAGTT
CTAACACAGGGTCTGGCATATAAGAACAAGTCAGTAAATAGTTGTTGAATGGAATTGAAAATTTAAGTAGCAAATAA
AGTATTTTGACCTACAAAGCAAGAAATCACATTTTTCTTTTTGTCACAGTTCCTTAGGAAGATAATTAATTTTTTAG
TATTTAAGGATGTTAAATATTTATTTTATGTTCTATTTACTAGGCTTCTTTTTATGAAAATTAATTGGTGAAAATAG
CGTACATATCTTCCTTTACCAGAACATTTACATTTTGGGCAGTAACGCTGGCTTTTGTTAAAAAAGCAAAATATGTG
TGAAATTTATGTTTGAGTTGATTTCAATGCATTACATTTCCATTTTAAATCTTCTTTGAAATACTCTATTTTTGACA
CCATGAAACTGTATTAGATCTTAGTATGTTAGCAATGTTTTGCAGTTTTAGAGCCATAATTATTTTAATGACCACTT
TCAGCATATACGTTTTCTACAGGAAAAATAATCTCAAGAACATGAAAAGTGAAATCTATATTTTGGGTTTCAAAATG
ATACATTTTAGCTAAAATATCATAGTTTTAATTTCTCAGTGAAAAATATAGTGTGGTAATTTATGAAGAGACTCAGT
GTTTAAAAATTATGACTCTATAGTCAAGTTTATGTTTATAGGACATAGGTTATTCAATTACATTTAAAATAATTAAT
TTAGAAAATGTGATCAATGTAACAAATTTTACCTGTTCTTTTCTAAAGCTAAATTTGTTGTTTGAAGTGTTTCTTCT
AAAATGCTAATGAACTATCAATTTAATTGTTGAGCTTAGAGTTAGAAACTTAATTATATTGCCAGAAATAAAGAAAC
AAATGGATCCCAAAAGATTCACACATTAGAAATGTATGCCAGGGAAATGCTTTTGAATGTGTTCAAGTCATGGCTTC
TAACTCGTAACTTATAACTTGTGTTATGTCTGGCTTCATTCCCTTAAGAAAAAGGAATAATAATGCCTTCGGAGAGC
ATCCCAGCTGTAAGAGCTATGCATTGGTGTCTAAAAAAGCTTCTCACTCCTCATACCATCCTGGTCTGGGAATTTAA
AAAATTGTCATCTTTTGATAATCTGTATCACATAGTCTTCTGCATAGTCATATGAGGTTAGAACTGCCCCATAACTT
TTGCAGGGCCTATAGTAAGTGTGCAAATGGTTGCCTGCATGCCACATATTTAATATTTATAAGGTATAAAGTCAACA
GACTATTAAATATATCCTATCTGCTTTCCTTGACAATTATACAATCATAATGATATGGACATCTAGATTCGATTTAG
AATTCTCTCTCTCTCATTTTCTTTTTCTTCTTTCTTTCTTTCTCTTTCTTTCTTTCCTTCCTTTCTTTCTTTCTTTC
TTCTTTCTTTCTTTCTTTCTTTCTTTCTTTCTTTCTTTCTTTCTTTCTTTCTGTCTGTCTGTCTGTCTTGTTTTTTT
AAATAGTGCAAGCAGTTTATTCCCTGGCAAGGAATTTGGAAAAAACTCAAATAGCAAACCACTTGATACAATAAAAT
AAATTCCTTAGAGTTTTGTACTGGAATGAGGCAGCTTGGTTAGAGCTAACCCTAAGCCTGTTATTTAGGATACATTG
GCTTTTCTTAAGCTTAAAAAAAATTTTACTGTGTTAATGACATTTAACATGAGATCTATCATCTTAATAAATACTAC
ATGCACAATACATTATTATTGACTCTAGGTAGAATGTTGGACAGCAGATCTCTAGAGCTAATTCATCCTACTTAACT
GAAATGTAATGTCTGTTGATTAGTAACTTCCTATTTCGCCCTATCCCCAGCCCCTGGCAACCACCAGTCCAGTCTTT
GATTTTATGAGTTTGACTGTTTTAGATACCTTATTTCAAGTAAGTGGAATCATGCAGTATTTGTCTGTGTCTGTCTT
GTTTCACTTAGCGTAATCTTAAGGTCCATCCATATTGTTTCATATTGCAGAATTTCCTTTTATAAAGGCTGAATAGT
ATTCCATTGTGTATATATACCACATTTATCTATTCATCTGCCAATGGGCATTTAGGTTGTTTCTGCATCTTAGCTAT
TGTGAATCTGTTGCCTTTTTTCCCTACCTCCTTTACTCCATCCTGCACTGTGAGGAACTCTGTGCACATAGATCTGG
TCGCCCCATTTCCCACCCACATGTTCAAGTTTTTCCCACTCACCTCATGCAAAGATTTACCCCTTAGCCATACCCAG
TAACTGACTTTGAAACATTTGCCCAGGGAGTTGAGGGATTCTGAATGCCAGATCATGGGAGCGGGGCTTCTAGTGAG
CATGTTGGCTTGGTCCTACAGACTCCTAATCAGAGCTTTGCCTTTGAAAGCATGGGGCCCAAGGGCAAGGACCCTAC
TTGTTAAGGTCTAAATTTTTTTTCTGAAATAACCACATCGAGCTTTTATGTGTAGATGGCCTAAATTGGGCTAACCC
AGAGGCAGTGACACTCAAGTAGTTTACATCTAAGCGCTTTCCATGTGCTTCTTTTCCCATTTCTGTTACTTCTTACA
AAATAAAAAATCAGCATCTCAATTACCCTGATTTGATCATTGAGCAATCTAAAAAGTATCAAAATATCACATGTAGC
CCCCATATACATACAACTGTTATATATCACTATAAATAAATATATACACATTATATTTAAAAATCAATACTTTAATT
TTACATGTTTAACAAATCACTAGCATATACATTCCAGATTGAACTTACGAGGGATGTGGAAAAGATTCAGTGACTAA
ATAACAATAAAGTACTCTAAAAATGAAAATGTGAAATGGAGACAGTATAAATCTAAAATCATATCACTTATGAAGTA
TTGTTTCAAATAAACAATAAAATATATCTTCAATCAATTTAATTTTATTTTAGTTGTATAAAATCTTTCGGTCAGCA
TTAACCTAATTGGAACACTAAATAGGTACATCTAAAAAATATAATCCCCCCCAAAAATATGTAGCTCATAAGAGATA
ATGCATTGAACACAGATAATATTGGCGTTAAAAACAGAACTCTACCACATTTGCAACGAAATGTTTATCTGTTCTTC
CTACTAGAAAATAATAAAATAGTTCTGCATGAGCTTGAACTCGAAGTATTAGGTGTACAAAGACCTTTTAGTGAATG
AATGCTAGCTGAAAAGCAAATTTTAAATATGAAAAATTAGCAAGACAAACATTTGAATTTGTGGGAGATGAGTAAAA
CTCCTATAAAAATGAATTGTTTAGTGTTAAACAGATTGTGTATGAAATATTAATGGCATATTGTCCTGAGCTCCCCT
TCCGCTGTTTCCATGTAGATGACTGAATTTCAAACAGAAATATGCCAGGAATGATTACGTGAATGAATATTACTACA
TGAGATTGCTTAAAGAGTATTTCTTCTTTTGCCTTCTTTTTACTTTCGTTATTTCATTTAGTAGTTAGAAAATACTG
TCTACAAATATGTGAGAACTGCTTAATTTATTTTTGAGACATTAATTAATTCAACTAAACTATATTGACTGTGTGAG
AGAGATTCCCTTGGTGAATATGTGGATTTTTGCGGTGGTAAGAACTCTCCTCTGGAGCGCAAATGGTATTGCTCTAG
GAATAAAGCATATACCTCAGGCCCAGATGAACCAGTGCAATCTACAGTAACAGGTTCAAAGATGACCTCATGACCTA
CTGTGGACTAATAAAAATCAAGGAGACCTACTGCAAAGGTTTCTGGGAAATTCTTTTTCTCTTGCGTTGAACTAAGT
AATATACATATGTGATAGTTAGAGCTGCAGCCTTTGTAATACCATGACAGAAGATAACCTGAAATAAGGCTGACAGA
CACAAGAGGGAGACCTAAGAGTACTGAGAGATATGGAGCAGGACCCCCTGATTGAACTTCACTTGCAGCCCCCTTCT
GCAGTTTTCAATGACGTGAACCAGTAGAATCCCTTTGTTTACTGTTTTTGATTAATTTGAGTGCAGCTTTATGTTAT
GAGCAACTAATAGCATCCTCACTGTCACAACTGCCCTCTATACGGCAGGCACTTTGTGATACTAAAGAAAGCAGTAT
ACAGAGTAGAGCCCAGTGAATAACAGGGCAGATGTTGCAATTAAACTGCCTGTTTAAATTCTAGCTCTTCCACTAGC
TAACTTGTGACTATCTAAGTAATTTAACCTTCCTATAATCATACCTATCTTGAAGACTTGTTGTAAGATTTAAAGCA
CAACAGTGCTACTATAAAACAGGTATACAGTAAAGCTTAGCTACTTTTTTATTAGGCCATATGATATCATTTCATTA
AAATCTTATAGCCATGCTATAAGGTATTATGATCCTCAATTTATAAATAAGACAGCTCAAGTTTTGGTCAAGTGACT
TTACCAAGGTCATAGAGCTAGAAAATAATGATTCCAAGTTACAAGCCAAACCTCTTCAATGCCAAATTTACATCATC
CCCCATTACTTGAAGTGTAAGATTCACATGGACAGAAATTTTTGACTGTTTGATCACTGCTATCTCCTTATCATCTA
AAACAGTCTCTGGTCCATATTAGGTGTTCAATAAATATTTGTAGAGTACATAATTTCCTTCACAGACTCCACAATCT
GGTGAAGGAGGCAGACATGTAAGAGAATTATTTCAGGATTCCACAGTTGATGCTGTAACAGAGCTAAATATAATGAA
TGGAGGAGGAATGAATAAGTTTGTCTGGGAGCAATGCTATGGCTATTGAAATAAGTCTTGCTCATGCTTTGATTGAA
ATGGTGGATATAGATCACACAACAAATAACAATTAGATAACAGCTTGTTGGGAGAAAGCGAGGATCAGTGTTTGCCA
TAAACATTTCTCATAGCTAATGTCAGGTGTTTGATTTCTCAACATTTTATATCTTTGACTTTGATTTTCTCTGTTTT
TATTTTTTAACTCCATTCTCAAGAAGTCTGCACATAAGAGTTTCAACATCTAGCACTTCATAACTCCGTCATCTCCT
CTCAGGCTTAGAGCAAATTCTGAGACGTGGATTTATCGTCGAGTGATTTCTTCCTGGCATTTTATCTCTGAGACCAG
GATCTGGTTGCTAAGCATGTAGACATAGAAATGCATTTCTTCATTGAACCCCATAGGTTCAAACTAGTGGATAATGA
GCACAATGTCAATGTGATTATTTGTAATGGGGGAAAGGTTACCGGAGAATATTACACGACCATCCACATAGACTAAC
ATTTTCCTCATGACTAAGTTTACTTAGCAAAACAAATTAAAAACAGAAGTTTGTTTAGCAGCACAGAATTGAAGGAA
GACAACCAGATGGTTATGAGGAAGATTCATCCAAACTATGCCAGAACTGAAAGAAATTAAGTTCATTCAGTACAAGA
ATTGTCTAGAATAAGAGAATCCATTTTGTGTCAGCACTTCCCAAGTTCTTGTTAATGCTACCTTAAGTTCAATTCAA
ACCAGGCAGCATTTATTACGTGTTGTGCTGGGTCCTAGGAGGACCGCGTTTTAAGAACTTACTGTGATCTTCTAGAT
CAAGTTTTTATTTCAATATTTCTACCTCATTTCTGATTCTTAGGTGTTCCTTATTTCCCAATTTATCCCCTGCAGAA
ATTGAGGCAATAAGATGTCTATCTTATTGCCTATGGTGTTGATTATTTATGTTATATTCTGTTTTGTGAAGTTTGAC
CTCTACCTAATTAAATTACATTTTCAATTGTATCTTGGATTGATTTATTCAATAAGTATTCTTTAATATTTTTGCAT
GAGGTCGGTCAGGTTTCATCAGACATTAGGAATTAATTATAAAAATCTCTAGATTGGTACTTGGAGCTTAAAGGAAT
AAGGTGGTGGAACGTTAAATGAGGAGGAAAGAACCAGCAGAGCTGGGATAAAATTCATCTCTATCATCTTCCCACCT
GCTTGATCTCTGGCATATAATTTACTATCCGTGAACCTCAGGTTTCTCTTCAGAAAAGCTGCAGGGTTGTTGGGGGA
AATAAGGCAATTCCTGGGCTTCAGTATGTTCAAAACAGAGCATTAATATTATTATAGACTTTTGATGATTTACACAA
TTTTAGCTTTTTGGCAAGACATATTTACTAGTACTAAGTAAAAGCACGTTGACTTTCTAAAATGAAAATGTGTATGT
GAGGATGAAGAAAAAGAAAGTGTTTTGTTTGATAATATAGCATTATAACACTGCACAAAAAAAAAATGGTATATGCA
GAGACTTCCATCACTTGCTTATGATGCCGCATTGGGATCTCATTAATAAGACACTTCCTCAGACACTTCCTTTGTGT
TCAATAAATTTCAATTTCCTCCTTTCCTTCAGTTCACTTCAAGAAGGACGGCAGCAACTTTCTTGTTGCCAAACCTG
ACAAATGTTTTTTAGTGCTGATTATACTCGAGCATTCTGTAGCAAAATGCTGTGGGTGAAAATGCCTTCCTTCTTAA
GGGAATTTAGCTTCTGTAGTACCAGAATCTCCTTGTTGAATGAACATGTACTGCCTAAGTCTTAGTAATCCCTCCTT
TTTGAGCCCATTTTCTGGCATCTCTCCCTTTAATATTCCTCAAAAAGTTGGATTTTTCCTGGACTTTTCATATTACA
GACTTTCCTTTGGTCATCCTCATCCATTCCGTGATTCCAACTACATTTTCCCTCCATCCTGGCATCTTCTTTCTTCC
AGACTTGTATATGCAACTGCTTCCATTCATACACTTGACCAACCTTTTAATTTCTATAAGATCAAAAACTCAGCTCA
CAAGCTTTCCCCTACCATCGAGCGGGGTTCTTCTTTTGCTTCTTTGTTTCAGACAATGGCACCACCATACTCGAGTA
AGGCACGTTCATTTATCAGGTCCTACCAAATCTACAATAAACTCTCTTGAATTTATCCACTTGTTTTCATTTGAACA
GTCATTTCTTTACCTGGGTAGCCTGCACCTTCTACCTGCATTGATTCAGCAGTCTCTTCACCACTGGCTCTCCCTCC
CTCTCCTGCCTCTCTTCTTGCTCCTTCAATTTATTCTCTACTCTTCATAGTGACTTTTATTAATGCAAATATGACCT
TATAACTCCCTTGCTTAAAGACCCACTCATGTTTGTCTTTGTATCCATAACTTCCGGCCTAGGGCTTAACGCATAGC
AGGTGCTCAGTAAATCTGTGGTAGATGAAAGAACAAGTTGTATAAATACTGAATGGTCTGATGTGCTCTTTGTTGTG
TCAAGAAGGACATTTTGCAGTCAGGATAGCTACATCAGTCCTTTAGTAGGCATTTGACAGCACTCGCATTATTCCTC
AAGAGAAGATGGATGTATTGATTCTGTATTTCAAATGACATAACTTTTGTGAAATAAGAGGCTGCCACGGTAATCTG
AGGGATCTCTCAAGTTCAAGGGACTCCACAGTGCTTTGTGTAAGGTAACAGGCTAAAGGGTTCAGTCTTAAACTTTC
TTAAGACTGTAGTTCAGGGTTCCTATGGTGGGGCTATAACCCTGAATTACATCCTCTTTCATTTCATGCTGATAATG
AGAACTACAAACCAAGGGGTATTAGGAAAGAATCCAGGTTTGATGCAGGGAAAAATAAAAACAACTGATAATCTCTA
GTGTCCCCAACTTCAAGAATTCCTTTCTTCTTTACACCAAGCTTTTTTTCTCTGCCAGGACTTACTTTGTCTTCTAC
ATGTTTAAGGGAGAAAAATGAGTTAACAGAAGGGGAGGTACAGCATTTCTATTTACTTAGATGCTAGAGAACAGGAT
GAAAGGTATGAAAAATATGAAAGTCTCTCTCTCTCTCTCTCCCCAGCCTTCCCCCGCTTCTCTCTCTCTCTCTCTCT
CTCTGTGTGTGTGTGTGTGTGTGCACGTGCGTGTGTGTGTGTGTCATAATACTCAACCTTTCTTTTCTTTCAAGCAT
ATGTTGTGGCAGAGACAAGTGTACATCAAAATTCGTGGTCCCTCTTTCATAGTATAGAGTTCTTGCTAGGATCCAGC
TGCAAGCCAGCAACTACATTTCCCAGCCCCACTGGCATCTAGTTAGAGCCATGTGACTAGTTGTGACCAATTGAATG
TGAGTGGGAGTTATGTTGCAGGCATACCTTTTCCATCTTCTTACTTCCCATTTGCTAACCTTATGGAAAAGAGTCCC
AAAGACCTAGGAGATGAAAAAGCCTAAAATGGAAGGACTCAGAGTCCCTGAATTACTGGGTAGAGAAAAGCTGTTTG
CAGATGGGAATGCCCATTTTGTAGTATTCTTTCTTTTCTTAAGCCACTAAAATTGTGGGATCTCTTTGTTATAGCTA
CTGGCATTAACCTCTTACGTATACATACAGCTATGTGCTACAAAGAGGAATAGATACATTTTTTAATCGTTGAAAGG
GGAGAAAGAAACATATTTAGGAGGAAAATAATTTAGTCTCTACAATTGAAAAGTGTTTTATGAATAATATTTTGTTT
TGGCAGCATATTAAATCTCAGGCAGCTGAACTACATTAATTTTCAATTCTCTATATATGTTTTTGTCTTCAGGGTTT
AGTAACACTGATATATAACAGTTTCTTTCTTTTAATTTCCAAATTTAAATGTCTAAGTTTGCCTTCTAGGCAGAAAT
TAAGTCCCATTGTGGAATGAGATTGGATCAACACTTCACCAAGATCATTTTAGTTCTTTGTAATCTTAAATGAAATA
AGCTAATAAAGCATTAAATTAGCATGTTGTAAAACTTCGTGAAGTTTTAATATGCTTCTAAGTGGCAGCTCTTAGCT
TATTATCTCTAAAGCTAAAGTCAAAATAAATGTCTCAGTTGATGAAATGGAGATGAGGCAACATTTTATCAAATTTA
ACAAAATATTTTATATCTGAATTATAAAGTCCAGATTATCTAGTAATTATCATATAAATGTATTTAACCAGACATGC
ATTTTTCTCTAATCAGTAGCCCTGGAGTCTTTGGACCACAAATGTGCCTTATCTCAAATGCTTTAACTGTGACATTT
TGCTTTAGACTAGCTCGACTACTTCTACAGAAATTATACACTTCATTCACATTCATCCAGATGAAAAAAATACATGT
AGAAATGATCATAATAAGTAACATTTGTTTAGGATTTCAGAGTTTACGAAGGGTTTTTCTATTCACTTTCTCACTTG
TTCTTCATGTAAACTGGTTTGGTGGACAACTGTCATTATCCCTGTTACCTGGAGCCCCTGGGTCTTAGGGAGACTTC
TTGACTTCTCAAGGTCATGAAGGTGCTAACTCTGACCGTGTTTTTATTCCTACTGTGCCACACTTCTCAGGTAAAAA
TCATATTGCAGACACTTTAAGAGAAGTACTTAAGAAAATAAATTCCTCCAGAGAATTACATTTAAGTTGTTTCATTA
ACTGCAGTGCATAAAGAAAGGAAAAGTGTTCCCAAACCCATGTAGTATTTTGCTATTGCTTATGGTAATATTCTGCA
CACCTAATATTGTCAGCATAATTTTCCATGTAACAAAATGTCCTAAATCAGCAATGTCCAATATAACTTTGTGTGAT
GATAAAAATGTTCTGTCTCTGTGCTGTCCAATACAACAGCCACTAGATACACATGACTACTGAGCAATGGTAATATG
GCCAGGGACACTAAGGAACTAAATTTTTATTTAATATTAAATAACGTTTAAATTTCAAAAGCCGCATGCGGCTAGTG
GTTGTCATCAGATACTGCAGTTATAGAAAATTAGAATTTACCTCTTTAAATACTAAACCTATTTTTAATAGTAGGAT
TTTTAAATTAAAATAGTTCTAAGTGCTTTTAAGTGATACGAAGTCAAATGCAAGATTTCTGTTTTAATAGTACTCTC
AACCCAGAGACAATCTTCATGCATCCTTATACATGTTCTTTGTTGCCTTATTCTAGTTTTATTTTAACATTAAATGC
CTCTGTTCTACTTGATATTGACTTGCTTCAGAGAACACCAAGTATAGTGGAAAGAAACACACACATGAGGACTTGAG
GCTACCAACCAGGTTCAACTAAATGCACTCTGATTTAATTGTAGTATTGGGATCCCCTGTTGCATTTATTGAAGAAG
AAAAAAACTTTGCAACCAAAAAGATATTTGAAAGCAACTGTTCTTCTTGGACACATGATCCCTCATAAAGTGGGGCT
TCCTGCTTTTCAGAGACTTAATTTCTGTTCATATTCATTTCAGCAATAGTAATAATGATGATGGCGATGATGATAAT
AATCATGATGATGCCTAAGTGTTGTAGTAATGCTTCTTCTGAGCCAGACGTTAGTCAAATTACTTTCTCTACATTAA
TTCAGGCAATCATCACAACAATCCCACAGGACAGGTTTTATTATTATACTTATTTAGCTAGCAAATGATATAACTAG
GTTAAGTTACTTGCCCAAGGTCATACTGCCAAGACAGTGGCTCTAGTGTCCCTGCTTCTGACCATATGTTATGCTGC
CTATCCTAGAGCTTTTCTCTTCTAAAATAGTAAAATAATATATTCTTTGTTTGTTTCATACTTTTTTTTTTTTTTTT
TTTTTTGAGAGGGAGTTTCGCTCTTTCGCCCAGGCTGGAGTGAGGTGGCGCAATCTCAGCTGACTGTAACCTCTGCC
CCCACCAGGTTCGAGTGATTCCCCTGCCTCAGCCTCCGAAGTACCTGGGATAATAGGTGCCCACCACCATGCCTGGC
TAATTTTTGTGTTTTCAGTAGAGACAGGGCTTCACCATGTTGACCAGGCTGGTCTCGAGTTCCTCAGCTCTGGCAGT
CCGCCCGCCTTGGCCTCCCACAGTGCTGGGATTACATGCATGAGCCACTACACCCGGCCCATACATAAATATTTTAA
GCGAAGTACACATGCATGATCATCATACTTTTAATAATTTCATTTAACTGTTTCCAAAGAATGTTAGTATGAGGTTT
TCTTTTTTTCTTTTTATAATTTCAACTTTTATTTTAGATTCAGCGGGTACATGTTCCCTGGATATAGTGCATGATGA
TGAGGTTTGCTATATGAATGATCCCACCACCCAGGTAGCGAGCATGGTAACCACTAGTTCTTCAACCCTTGCCTGTT
CCCTTCCTCCCTCCTTCCTCTGTAGTCCCCAGTGTCTATTGTTCCTGTCTTTATGTCCATGTGCACTCAATGTTTAG
CTCCCACTTTTAAGCGAGAACATGCAGTACTCGTTGTCTGTTCCTGCGTTAACGTGCTTAGGATAGTGGCCTCCAAT
TGCATCCATGTTGTTGCACAGGCCATGATTTTGTTAGTTTTTATGGCTGTGTAGTATTCCATGGTGTATACGCGCCA
CATTCTTTATCCTGTCCACCATTAATGGGCACCTAGGTTGATTGCATGTCTTTGCCATTGTGAATAGTGCTGTGATG
TTATATGTACTTTTTGGTATATTCAAAGAGAAATGCTATTTTCCTCTTGACATATTTATGTCAATTTAACATATTTA
TGTCCCTTTTCTTTTTAGGAGCACCATTCTCTTCCTTTAACATTATAAATAAAATATTTTTTGCTTTTCTGTTTTTG
TAAGTGCAGTTTTATTGACAGAGTGAGACATACACGTCGATATTGTGACTAGCTGCATGTCTTCTATTATTTAGAGG
TCTCACTCAAATGTAGATTATCAAATTCTGTTAGTGAAGAGGGTAGAACAGCAGAACTAATGCTGGTTTCCTTCTCT
AGCATTATTTGATGATAAACTAAGATGATAATACCCCCCAGGTCTTAGATACCTGCAGTAGGACAGGCACCCTACAT
TTAATGCTCCTAGGAATCCTTCAAAGTGATAGCATAGTTATTATACAGTAATTGAGAAAACTGATGTTCATAAGTTA
GAAATTTTTCCGAAGTTGCAAAGAAAGTGAATGGAAGAATTATACCAAGTTCTGGCCGGGCGCAGTAGCTCATGCCT
GTAATCTCAGCGCTTCAGGAGGCCGAGGCGGGCGGATCATGAGGTCAAGAGATTGAGACCATCCTGGCCAACATGGT
GAGACCCCGTCTTTACTAAAAATAGTAAAATTAGCTGGGCGTGGTGGCACGCACCTGTAATCTCAGCTACTCGGGAG
GCTGAGGTAGGAGAATCACTTGAACCCGGGAGGCGGAGTTTGCAGTGAGCCGAGATCGTGCCATTGCACTCCAGCCT
GGGCGACAAGAGCAAAACTCCGTCTCAGAAGAAAAAAAAAAAAAAAAAAAGAGGATTATACCGAGTTCTCTTTGATT
CCAAGCCCAAACAAATCCTTTTTTGCAATATATGACATTGTTTCCCTGTTTGCATTCCCCATTCTGTGTATCACACA
TCCTGTGGCCTGATCAAAATTCATTTTCAGATTCTGAATTTATTTTCCATTGAATCTATATAAACTATAAAGACAGA
AGATATATGTATGTGTGTATACCCACGTTTCTCTTCCAGTGTCAACTGATAAAAATAGATTTCAAAGTCTCAATAAC
CTTTAATTCCCTTTTTCTCTTAAAAATTCTTTAGAACTTGTACATGACATTCTGACTCTAGCAGATTTTAGAAAACA
GAGAGGCCATTAGATATTCATACCTTACTATTCAGATGAAGTATTCAATGCTAAATTATGTAATTTATCTGCTTTGC
AAATTGTATGGTCAGATTGAGTTCCACAAAGGAGAGATAATTTTTAATATAGGCATTCTGTAGCTTCCCTAATTATT
GAATTAGTTTAGAGCAAAATCCTTAAATTGTATCGTTGCTATGCTCAAATTTTGTATACTTGTCCACGTAGGCTATA
TTAAGATTTCATTGAATTTTGGTTTCTTTCTCAGTGATAATTCAATATATCAACTCACCACTCAGATTTGCCTTTGG
GAAAATCCAGGCCCCTTTTCTGGATTTTTAGAGCAGATTTTAAAAAAGTGATTCTGTATATGTGTTGAAATTAACCA
CATCTCATTGCTTTTGAATGATTGAGGTAATGTATACCTACTACTTTAAAAAAAATGACTTACTTAGAAGGTGTCCA
TAGTTTTATAAGTTCCATTGAACTGGTTTATATTGTATTTAGAAAGGAAAACTACTCCTTTTATCCTTAAGGGTGAA
AACCTGGATTTTATTATACAATTAACACATATTTATTTTTTATTATGAAATATATCACAATATAAACGTTTACAGGG
AGTGTTTAAAGTGGTGTTGTCCAATGGAAATATAATGTGAGTCAAATACGTAGTTTTCAATTTTCTACTAGCCATAT
TAGAAAAAGAAACAGAGAAATTAATGTAATAGGATACTTTATTTAGCCTAGTATATCCAAATCACAATTATTTAAAT
ATGTAATCAATATAAAAATTACTAATTATGTATTTAACCTTTTTCTTTAGTAAGTCTCTGAAATCTAGTGTATATTT
TACATTTATGGCACATTGCAATTTGCATTAGTCACATTTGAATTGTTCAATAGCCACAGGTGGCTAATGGCTACCGT
GTTGGACAGCACAGGTTTAAAGAATAATATGAACATCTGTGTTCCAACATTCTGAGTTTCAAATAAGAAGAACACCA
TCAGTATTTTGGGAGAAGCTCCCTATGTTACCCCTTGCTAATCACCTTCCTTCCCCCCAGAGCCAAAAGTAACCATT
ATCTTGAATTTCTAGTAAACAATGCTCATTTTTTAAAAAACGTATGTTCAACACCTGTATTTGTATCTTTAAAGAGT
AGCTAGTTTTAGTTTGCCTGGATTTGAACTTTATATTAAGGGAACCACCCCATCTCTAATCTTCTCTGTGAATTCTT
TTCTCTCAATACTATGTTTTACATATTTACGTTCATCAATGTGCAACTCATTGTATGTATATAACACAATGTATATA
TTTTACATGCGTATGGACATTTGGGTTGTTTTTATGTTTTTGTTCATCACAAACCACAACACACATGTGTTCTTGTA
TATGTTTTATAGTGCATGTTTAAAAATTTCTCAACAGTATTCGCTAGTAGTATTGTCAGGTCATAGGGTATGCACAC
ATAAATAGAAATGATTGATTAGCTGCAATTTGTAGTGCACACATATTTGCTATGTAAGTGATCCATGTTTAAGACTT
TAACTGAATTTAAAAAATATTTTATTGGAGCCAATCTAAATGAGCTAAGGGTTTGTATTGTTTACATAAGCAAAGAT
TACACTTACTGGGTCAATTCGGTTGATTAACTTTGGATATATAAAATATATAGCTAGTTGTTAAATAGATATAATTA
TTAATTGGCATTACTTTTGTTTGTATATAAAAATTTCAAAATATCCATGACTTAAGCAAGGTAAACACCCACTGGGT
GGCTTAAGCAACAGAAATGTATTTCTTGCAGTTCCGGAAGTTGAACGTCTAAGATTAAGGTGATGACAGGGTTGGTT
TCTGGTGAGTCCTCCCCCATTGGCTTGCAGATAGCCGCCTTCTCCTTCATGACCTTTCCTCTGTGTATGTGCATCCC
TTGTAGCTGTTCTTCCTTTTATGAGGACATTAGACTTATTGGATTAAGGTCCTACCCATATGAACTCATTTAACCTT
AATTACCCCTTTAAAGGCCCTACCTCCACTTGCAGGGGTTAAAACTTCAACATATGAATGGGGTTGAGGAGACCTAC
TTCAGTCCATAACAGTTTCTATATTCTGAAGATGGTCTTTAATTAACTAAACAGTTAATGTTACTTTACTGGGAATG
TCTTTTGGATGGGGGAATAAGCTGATGATATGAGAAGGGTTGGTGAATTTCTCATAAGTGTGAAATTTGTTGGGCCG
GCCCAGCATGATTTTCAATCAAATACGCTTTGGGGACAAGTAGGTTGAATCACTACGAGAGGTTTAAAAGAAAGCAA
GTTGTAATTGCAACTTTTAATTGAAAGAAAGACAGGCTTTGTTGATGTGCCAGCAAGACTGATAACTGGCTTTAACG
TAGATAGTAAGGCAGCAGATTCAATCCACTGATCGTGATCTACTAGTGAATTTCAAAGCCTTATGCAATAGAACTAC
AAACCCTTTCCTTGCCCACCTTGCAGGTGGATCCATAGGCAAAATGAACATTTGCAAAAAAGCCGCTATGTTTCAGA
ATTTGTGCTAGGGCTTTAATATCTATAATTTCTCCAAATCCTCACAATTTAAGAATTAATTCAACTTAGCCCCATGA
ATAGGGTGAAAATTCTGAGATTTAACAAACTAAAATAAGTTATCTGAAGACAGACAAATAGAAAGAGTTGAGATATT
CTATTTGAATGTAAAATTTTCAAAAAGTAGAATGACAGCGTCAGGAATTACAGTCTCAGTGTTGAACACAAGACTTA
GGAACAAATTTGCTGCATGTAATTTCATTGAGATGGGACAAAGTACAGCATACGTAAGGAAGTTTTAGAACAAATAA
GATAATTATTTTACGAGCTTTGAAACATGTGTAAGAAAGATACGAATAAAAGTATAATCACATTTGACTAAAACATG
AATACCTTAAAACTGAAAAGCACTGAGATTATCATTATATAATTTTGAATATTTTAAACCACAATGCTTTGGGAGTG
CACTGTAATATTTTAGAATTGGAATTTTAACTTACTGGCTTAAAAAGTAATGTACTTTGTTTTAAATTCAAAGATTA
TCTTGTAAATTCAGTTCGATCTATTGAAAAAATTATAAAATTCGGCAAGAAGCCAAAGAAGAACAATTATGTAGCTC
AAGATAATTAAATTTTCATGTTTGGCTTTAGAAATATATTCGTCGTGACATAGTACATGGTAATCTAGTGAGCCCAG
ACAAGTAGTTTTCTCTTTTTGTCAAAGGGAACAATTTGATGCGTGTTCAAGTTGCTTAAATAAAATTTTGTATGTGC
TTTCTCATCACAAGAGAACAATATGATTTTTGAAATTATTTTTACTTTATAAAAGAAAAAAAAAAGCCCTCACAGAG
AAAAAAGAAAAAAATGATGATGTCTTTGAAAAACAAAGTTAATACAGCTTTACATATATTTGACCTACATCAGGGTT
AATATTTTTCAAGGTGAAACATTAGATGCTGGAACTTGCAAAAACAGGCAATCCTCCTTTAGATGAAACGGACACTC
TAAGGGTTAATTCATTCACTGAGACCTATTGTGAAGTAAGCCCTACAGAGACTGAAAAAGTTAAATGCAACTCACAA
AAGTTGCTAGAAGAGTCATGATGTTAAAATAAAATAAGTACACAATGTATGCTGCAAGTATACTTAGAGCCATGCTA
GGTGCGGTTGAGAAGTTCAATACAGGTCCAAGATAATAGCTGCTTCTCCTATAGAACATGTCTTCTCATTGGAGGGA
TAAGACCTGTGTCTATGAAACAGGCGTAATTACATAGCTCTGGAACTATATATGCCGAAATAAATGAGACAGTAAGT
GTTATTGTACTATAAAGAATGAAGAAATCATGATGAGAAGTAACAGTTAATGAATGTTTTCTAGAAAGAGTAGGATC
TGAATTGGCCTTAGGTTGTAAGCAGAGTTTATAGATAGAGTAGTGGTATGTCAGAGTCACTCTGGGTGCTTAAACAT
ACAAATCCCCAAGTCTCACCCAAATGTGTCTTCAGATGAAAGGAAAAAACAAATGACTTGAGCTCCCCCGCAAAGAA
CACGGGTGGTATATTGAGCAGCCAAGGAGTGACCAGAGTGGCAGGCCCATGTTGAGGGACAAAAGAGGACAATTAGA
ATATGATTAATACAAATTTACAGTGGGATGAGTTGTTAGCCTGAGGAGCTTGAATGTGAACCTCTGTGCAAAAAGGA
GTCATTAAATACTTTTGAAAAAGGTGGGATGGGAAGAAAATGACATTCTCAAGACAATTAGATCGAACAGTATTAAG
CATGCTGACTTATTAAGTTATGCACCTTGAGAGGGTGGAATGAGGGAAAAGGGTCTTTATCTGGAGTAAGACAGGAA
GAAGCTAAGCTGTAATTCTTACTGGACTGTAAATTATGTGCAGATATATTATCTGTCATGTTCGTGGGCGCATTCTC
AGTACATAGCACTTGAAACAGGTACTCGATAAATTGTCAAATGGATGCATGGAGTGATTTCCATGCAAAATCTAATA
TTGTATAGTATTAGAAGGGGGAAAAAAGCATGGCATTATGCTAGCAGAAATGTCATTTGGTATTGAGGATGAAACAT
TTTCAACAGTTTGCAAAGCCATCCACTCAAACATTCTGTCACTTTCCAATAATTTTGAAGGATGTTCTTTCTACTTC
TACCTTATTACACAATGAGTTGAGTAAGATAAAGAAGTCATGTGCAACAAAACAGAGGGAGATTTTCTGAAAGGCAC
TACACCAGGAAGTTGTTGTACTCTTGCTTCATCTTGCCATCTTGGATATACTTCTGGCGCTACCTCCAGGCCAGTTC
CTCGTTACATATGTCATTTACTTCCCACATGCTAGACTCACCGAGTTAATCATTTTGCTGCAGTTAACACATTTTAG
CAGAGTGTAGGTTTATGGGTGAGAAGGAAATCAATGATGTTTCAATACAGGGTTCTTTTCCCATCCCCCTTATTTCC
ACTTAGAACTGTCTCTCAAGTCTTAATTTGCCTCTAAACTTTTTTCCCAGCTTACATTCTTTTCTGAAAAATGCAAC
GACGATGCCAATGTTTGTTGACCTGAAATACATTGTAAAACATTCATAATACTTTGAGCAGAGCTTCCAAACTCCCA
TTTGCCTCTTTTATCTCCCTTACCTTGGCCCCTTTTTGAAGGCAATGTGATATTTAATCCGTTTCTATTGATGCTTC
AAAATTATTGAAAAACTGGTAATTGTATTTTTCCCTTTACTTATCAGTTGCTAGTTGACAATGAGTGTTTGCCCAAA
CAATAACCAATCAAAAGGTAAAAAGGAGATTCCAGACATATCTGAGAAGAAATTCTTTGGAAGAAGCCCGTAAATGG
AATGGGAATTCAAACAAAGCCGTTTCCAAAAGAAATACTAAATGGTCTCTAAATGCAAAAGGATTGCTCCCCAAGCA
TTTTATGGGAGCATAAAAAGCTCCCAACACATTTTATGACAATACTTCTACTCAATGACTTCTTGTGTTGACATATT
TGTTGCACTCGACGTTAGTATTTACAGCTTCTTATCCCAAATATTTACTTAACTGAAGCCCTGATGTTTTTAAAAAC
TTTTCATCTGTGTTTAACAGCCCATTTTACAGAAACTTATTTGTTTCATCAGGCAGATATTTACTGAGAACTTGCAA
GTGCCATATATTCTAAAAATGCTGATGATAAAACTGTGAACACAATAGATTCTCATGGTGCTTATGGTCAGGGCTAG
CACACACACTTGTGAAATGATCACTGATGATCAAAGGCATAAACACTACATTTGGAAGAAATACCGAGGGATCCAGA
AGTATCTTGGAAACACTAGCAAGTATAGCAGATGGTGGGATTGGTGCTTCAAAGAACTTCTTGTGGAAGATGTTACG
TATGTACCTTCTCTGTGCCAGGCACTGCTAGGAAGTGCTGGAGAGAAAAAGATGTGCTAGATACCGCCTCTGTCCTA
TGTGCTTGTGCTTTGTGGGGAGGTGAGTAGGATAATCCCAGTTCTCATGCAGTGTAATGAGTACCATGACGGAAATG
CACTCCAAGAACTAGGCAGCATGACCAGAGATAGGACATTTGAGAAAGACTTCACTCGGGTGGTACTATCTTAGTCT
GGGTGCTAAAATAGATGTGATAGATGAGTAAGGGTGACCCGGAAGCAGGAGGGAAAGGGAGGGGCTTTCAGAACAAC
AAGTGCGAGGACATTAAGGTGAAATAGAGTATAATAGTATTCCCAGATCCTTGGGATTGTTCTCCATTAGGCTAAAA
CAAAGGTGTTTTCTCTTCTTTAAGATTTCATGACTGCAGATTGCATAACAGAAGGTCATTTAATAGACCTCTAAACT
GAAGGAATTCTTGAATTAAATCACAACATATCTTCCATGGCCAGAGAAACCATTGCCTCCTTATGTCGACATTACTA
ACAGCACCAGCACCTGCTGCTCAGGCCAGCGGGAGGGTTGGGTGTTGCTGCCTAGGTAATGCTCACCAACTGATGTC
CTGCCATGAGTAGTTTTGCCAAGTTCCACAAAAAAAACTTAGTGTTCTATCAGCATCTAATGAGAATTACAGTCATT
AGTTAAATAAAAGAACTATTAGATAAGGAGCAGAATGAACAACACACAATCCATCAGCTTGGTGAATGGTATCAGAT
GGTTTCTGGGTGCTGGGCAGCTGTGCATCCAAGTAGACAGGGAGAATATATATGTCCTTTGCCTTATGTACTTGTTT
CTCTAATCCAAAGGCACAGCAATCCGTGGAAGCTGCTATGATAAGGTGTTTAGTGGTGAAAATGTCTTGAAAGCCAG
TAGATTATTAAAGTGATGTTTTTAAAAATGCAGATGGAGAGTAAGTACTTTTTATCTAGAGTAGTAGTTCTCAAAGG
GAGGTCCCGGGATCAGCAGCGTTAGCATCACTTGGGAACTTAGACCTGCATGGGCCCCATTCCAGATCTCACTTGAA
AACTCTAGGGGGTGTAGCCCGGCAGTCTTTGTTGTGACCAGCTCTCCAGGGGGTTCTGACACTCCAAATGTTCAAGT
TTCAGAACGCTACTCACAGGCCATCATGCTCGGCATCACCTGAAAGCTTGTTAGAACTAGAAAGTCTTGGCCCCACC
CCAAGCCTACTAAATCAGAGTTTTTGGGAGTAGGGCCAAGAAAACTGTGGGTTAACAAGGTCTCCAAGTGATTCTTA
TTCATGTCAAAATTTGAAAAGCGTCGATCGAACTGTTGGTTCTCAGCTTTGATTGCGTATCTGAATCACCTGGGGAG
ACAGTTGAGCTATTCCGGGCCCAGATCACATCTAGACCAATTGAATCAGAATCTATGGAGGCAGGACCCAGACATCA
GTATTTTAAAATATTTCTTGAATGATCCCAGAGTGTAGCTAAGGTTGAGAAACACTGTTCTAGGATTAAAGGATTAA
TGTGTTTGAGAGTATGTTAAGATCTTAGGCAAATCACAAGGGTGTTAAGAACTACCATCTTCGCAAAAGGAGAATGT
GCCTCAGATATTCTGGTACTGCTTTGATTTTACCTTCAGTAGTCTTACCTATTTTGAGTATGCTTAGTAGTACTAAT
ATGAGGCTTATTACTAATATGTTAAAATTTGTCTTTTAATTAAGTGGGTCTAAACGTTTTAATCTTTAATCTCTGAC
CCAACTAGAACTTTTCTAAACATTTTCATAATAGTCTCCACCTTGTCTTCTGACCTTCACTTATGTTCTTTCAGGGT
TCTTCGTGTGTTACTAGTAATAGTAATGGCAAGTGTTTATTGAACACTTACTATGTGAAGATTCTAACTGGCTTTTA
ATAATCACATCAGCTCTGGGAGGTAGAAGGTAGGGATCCTCCTTGCTTATCAGGTGAGAAAACTGTACTATAGAGAA
GTTAGCAACTTTTCCCAGGTCATAATATGTGACAGCTAAAGGGAGCATAATGGTTGGAATAAAATAAATCTACTCTA
GTTGTACCGAAGGCTCATATTTGTCTCACGTACTTGATTTGGTCGAGGCCCAAGGGGTCAATTTCCAATGCTTGGAT
TCCTGGATATGTAGAGTTGTATTAAAAATGCTAAAAACCTATTATGTATCATACAATCATACATATCACCTAAAGTA
TTATGGAAATGAATCTGTATTATTAAGGGAAAAAGGCCTGTGTGAAGAACAACTGAAACTTCATTTTAATTGAAATT
AAATAACATGCATCATACACTAAAAGTGCACGTTATGACCCCATGAATTACTTCAGGTGGCTTTGATTCATGTTACA
TACACTAACAAATATAGAAGAGTGATATAATGCTTCTTAATTAACTACTAATGGAAGTTTACTATTTAACTGCTTCT
TATGTAAGAATGTAAATGTTTTCTGAAATATCAGAACTTTTCATTAGGAAGCACTTTTAAAAATAGCAAAACTGATA
TGCACTATGATTTCCATATACATTAAATTGAACTTGTAAATGATGTTATAAATTATAGAAACCAAGGGGATGTTCAA
ATTAGATATTTGTCTAAATAAATCATGTATGGATTGAACAAATACTCATTGAGAAATAAATGTATTCCTTTTCTTTC
AATTATCTAGGATTCCTTGTTTATCTCTTCAGAAGCAAAATGTCTTCTGTCCGTTTTATTTCCAGTTAAACATTCTT
CAGATTATGTAAATAAGTTAACTTCCAATCCTCTTATTTCTGTTTATCTCACCACTCTTCTAATTTAGACGTGATCA
ATATCTTATCTTTTTGCATTTCATAGACATCAGGATCCAGAATAATTGAGTGAGCTCAAAACAACAATGGCAAGAAT
GATGTTTTCAGAAAACTCAGCAATCATTCGTTTAATAAATATTCATTGCCTACCAACTATAAGCAAAGTATTGGCTA
GGCCATGTGGGGTATACAAAAATGTATTAAATATGGCTCATTCTCCCTAAGAACTTACACCTATTAGACAAAGTACA
TGCATAAAAATTATAATGTATAATAGAAAATAAATACAAGCCCTAGAATGCACAGTTGAAGTACGATTTGCATTTAT
TATAAAAAGAAAGATGAATTGGCTGGGCACGGTGGCTCACGCCTGTAATCCCAGCACTTTGGGAGGCCAAGGTGGGC
AGATCACGAGGTCAGGAGTTCGAGACCATCCTGGCCAACATGGTGAAACTCTATCTCTACTAAATATACAAAAATTA
GCCGGGTTTGGTGGTATGCACCTGTAATCCCAGCTACTTAGCAGGCTGAGGCAGGAGAATTGTTTGAACCTGGGAGG
TGGAGGTTGCAGTGAGCCAAGATCTGGCCATTGCACTCCAGCCTGGGCAACAGCAAGATTCCATCTCAAAAAAAAAA
AAAGGAAAGAAAAGAAAAGATTAATTTCCTGTTAGCTAAATCAAGGAAGGCTTCATGGAGAAAAAAATATTTCAACA
CACACTTGACGTAGCAGTGGGATCAGGCTGATGTTAGGGAAGAATGAATGACATTCTACACTGAGAAAGAGATATTC
AGTATATATATGAAGAGCAGTAGAGAAACTAACAAGTGGAAATAGACTCAATTTACAATACTTGCCTGCCTGGAGTA
CTCTATACGTTGACTGTAAGTTGCAGTTTACTCAGAACAATCCCACTTTCTACTTGTTTATCCTATGTAATCATTTA
TTGGGCCTCCTTTTGCTCTCAAAAATATCCTTGTTTGGATAATAGATTATCACTCTGTTCCTAAATGAACTGCCCTG
TGTCCTATCCCAGTAAAAGGGTGCATTCGGGCCCTTCGTAACTGCCTCCACTACATGGTTGATTGAAACCAGAGCTT
GGCATTAAGAAGTTAGCTGAACAATCAGATTTCTATTCTTGGAAAACCCAAGAATTTCAGAATAGATACAGAAGCTG
TATAGCTTTAATAACATGACAGAGTTGTAGCCTTGAAAGCTATGTACAATTCAGAATTATGAGGGAGAAGAAATTGA
AGAAACAGTAGCAGCCGGGTAAATGCAGAAACAAATGAGGGAGACACCTAGGGGGTGACTGAGGCACAATAATGGAA
GAGAAGTGCAGTGAAATTGCTTGAACTCTTACTGATGAGATTTCTACTGTTGCCTTGAATCCAGGACCACCTATATG
TTCATTCTTTGTCATGCTCAGAGTTATGACAGATGCTGTTATTGAATTCCCCAGAGACTCCCTTATCGTCTCACCTC
AAACCTTACAATAATCCCTTCTATCTTTCTATCCATCCAAGCTGGCTTAAGTAAAGTCTATGATCCATATTCCTAGT
AAACAGAGAAGGGAAAGAGACTGAAGGCAAAGGCCCCAATTAGTAGGCTATTGCAATATTTCAGGGAAAAGGCAATG
GCCATCACATTGTTGTCCCAGGAATGAGAATAGAAATGAAAGAAGATAATGAAAGTTGAAAGGACTGGGGGGGCTTG
ACAACTGTTTAGACTTGAGGAGTCAGATAAAATAGGAAGCCAAAGATAATTCAGAATATTTTGATTTTGATTTTCAT
CACCAAATAAGATAGTAGTACTATGAAGAAAAAATGGTTAAAAAACAATAATAATAAAGAGAACTCCTCCAAATAGT
ACCAAGGGAGGGAGTTTAATAGAGGAAATTAATTCCGTAGGTGATGAGAGTCCTGAGAAGCCAAACGAGAAAAGATC
AAAACAACCCAGGGATTGGCAGTCGCAGGAAGCTGTTCTCACTTATGGCTGGGGCTTTAAGCACAAGGTGACATGAG
ATTTCAGAATTTGAAGTCGTCTGGAGGCAGCTAGGATCAGGTGGGGCCTGTCCTGTTCGGCAGGACCTGCAACCACA
GGAGGAGGATGCGTCAAGCAGAAAGTTGGAACACAAGAGGGGATTCAGCCATAAGCCACAAAATACCTTCCAGAGCA
GAGAGAAGGAGAAATACCCTGAATTCCGTATTTTCCCTGCCATTTAGTTCCCTGCTATTGCCACACATTGACGTATT
CCATCCAGAGAAGTCCATTGGCATATGAGTCTGGGAAATGTAGTTCCCAGGGGGACATGATCTTAAGGGAAATAGAC
AATGACTGGTGCAACAACTGACCTGTGTGAGGCAGGAGGGAAAAAACAGGAATAATATAGTTTTTCTCTAGATCCCT
TCATGCACAAAGATGCAAAAGAAATGTGTTGGCTTAATGAGCCATTCTGGGTGGCCCTGTAGGTGGCTGTCCTACGA
ATAAGATTTTTAGACAAAACAGAGATGACTTCAAATGTCACAAGAAAAGTATCAGACAGGAATTAATATTGACTTGA
TCTGTCACAGGCGTCAATGATTTGCATTAAGCCAACGATCTTCATTGTTAATGTCTGGGAAATTGCCAGCAGCATTA
CGACTACTTGTGTGGATTAGTGTAACGGATTCCCCCACTAACATTCAGGAAATCATGTCAAGCACAGAGTGCCTATG
TAAGAGTGGTTGTGTCTATTCACTACATTTCTTGGACTAATAACACACTTAGCCTTCCTGAATTGCCAACATGTACA
AAACCAGATTGGGGTTTTTTAGTTGTTCATGGAACTATCATTTATTGGGTAGCTCCTGTAGAAGCAAGATACAGAAA
CTCTAATTAGGAATAAGACAGTCCCTGTACTTCAAAGAGCTCTCAGGGGAGGCACACAAGTAAACAAGCAATTATTA
TCATACGTTAGGATAATACCGTCATGGTGATAACCACTGAGTGATAGCCAAACACATGGAAGAGGTACCCAAGTCTA
ACTTGGGGTAGTCAGAGACTGCTTTCAAGGATATCCGAGTAAGTGTTAGCTAAGACATGATACGTATTTCTAGGAGG
GAAATTTTCAAGGCAAGGTGGAGATTGTGCAGTGACGCCCAGAGCCTGGATTATTTTGGTGACTGCTAGTATTTCAG
AATGACTTCAGCAAAAGTTGTAGAGAAGATAGAAGACAACAAAGTATAAGCAGAGGCCAGATAATGAGGACCTGGAA
CAGTGGTTTGCTGGTAAATGTTTAACAAGAGGCTCTTGGCGGGGAGAGAGAGTGTCTGATTTGCAGCATTTGGCAAA
TTTTGTTGCACAAATGCTCCAGCATAGCCAATTTCAAGCTACCAGTGTGACGTCATTGAATGCAGAATTGGAAAGAA
ACGGGCAGTAGCACAGCATTGTATAGTTATTTTCATTACCCAGATATAATAGATAAAATATCCAGATGGTATTTAAT
AGATATGGATGCAAAATTTAAATATATGTACATTCATGTGCTTCATGTTACTGAATGCGCACAACATTCATTATCCA
TTCATTCACGTGTTAATTTAACAAACATTTCTGAGCCTCTGCTCTGTGCCAAACGCAGTTCTAGCTGCTGGAATTAC
AGCACTGAAAAAAAAAATTTGTCCTCACTGAGGTAAGACAAACATTATTATGCCCATTTTACAGCTGAGAAATTAAG
ACATATGAGGATTAAGCAGTATAGTTAAAATCACACAATTGGTACATGAAGGAATCAAAGAGGAAATCAGCTCTCAG
ATTTTAAATCCAGGGACTCGTTTCTGCTATACCATACTACCTACCTAGTTGAGCTGGATTTTATCATGGTTTCCCTA
TTTTTATCACCATGTGGTTGGATAAGTAAAATAAATATATGTGACCTTTCAAATAAATTTGGGTCATTTTTCTTGGA
AGCTCATCTGGTGTGAACTTTAAAATACTGCAATTAATAATGATTATAATACCCTGGAACTCTGTAGCAACCTCTTT
TGAAGAACTCCAAGGAGCCTCTAAATGTATCAAACTAAGTTCTTCAAGTGAATTAGTTATCATCTGAGAGTAATATA
GACTTTTAAAAATGCATTAATTGTATTAACCCTTTCAGGCCCATAGACTTAAGTGTTTCTTTCTCCAAATAAAAATA
GTAATCTCTGTCCATTTTCTTTAGAGAATAATGAAGTAATTTTCATTGAATATGTAGTCAACATAATTACTTCAATT
CAATCGTGAAGGATTTTAAAAATTATTTATGTCTACTAACTTAAAGACATGCATAGATTTCAAGAACTTAAAAATGC
ATATTGCCTCTTTGCCCTATGCCTCATAAAACAAAATTATGATAACGTTGTGTGTTACAGAAAAACGCACTGATTGT
AATGAAGGGTGCTTCAAAGGCCATGAACTTGGAAAGCAACTTATTTACAGAGACCCCCAGCAATAGCAGCTAAAAGA
TTGACTGACTCCCTTTATTTTCAGTTATCCTTCAGACACTTTTGACCTCTTCCTGTGCCTTTCTAGTCATGTGCAAT
CTTGTGGATATCTCTTCCTTCCTCTTGTTATTTTCTATTTCCTCTGTTTCTATTTGTTTCTAAAAATAATCATGTTT
GAATATAGGATTAGCTTCCTTCCCATCTCCCCATTACCAATCTCTCACTATACCGCTATGTTATTAATCTTCCTGAG
AAATATATCAGGTTCATTACATTAGTTACCAGCTCAAAACGTATCAGTGGCTTTCTAGTCCTCACAGGCTCAAGTTA
ATCTGCATATTCTGACTTTCATATTCTGGGTTCATGCAAACTTTTCAACTTTCCCTCTTATACCTACTTAGGAGGAC
CCTCAGGTTCCATCATGCTCATGTTTCAAGCCAGAAGTTCTCCTGCCTCTTCCTCTATGTAGACTCCACATAGACTA
TGATATCCTGCTTCTCTTTTAATCCTCCATCTTCAGCTCACAGCCACACTCCTCTGTGAACAGTTAAATGATTCTCC
CACCTCTTACCTCCTATAGCACTTATTTTTCATGCAGCATTTTTGAGACTTAATTAAATCTACAGTTTTAAAAAATG
TTTTTCTACCACAGTCTCTTATTCATACTAAAACTTTCAAGTCTATCCATTTTGCTTATACAACCACACCGTTAGGT
CTTTTAGGTCCAAGAATACAAGAGAATGGCAAAGCACGTTGTTTACATCCACACATACTGTGTAAATTCAGGTAATT
TTTTTTAATCCTATGATCCTCAATTACCTCACCTGTAAAATAGGTACTACTCATACTGCAGAACTCTTGTTGGAATT
AAATAAATGAGTGTATTAAAAATGCTCAACAAGATTTGGCACAAAATCGGTACTCAGTAAATGCTAATCATTATTCC
CTTTCTCTTCAAAGCTCCACAATTCTGTATTCATATCACCCTCTTTATATCATTTGCAAAAATGTATCCTATTCCAA
CTCTTTCCACCTAGCCTCAACATTTACAAACACTCCTGGTGGGAAGGGAAAGCTTTTGAGGAGAGCACATCTATACT
CATTTACTTCTCAGGGATGCAAGCTGCCCTGCTTACTGAGGGCATATGTTCATAGTCACACCGGAGCCCACTGTCCC
CTTATACTCTCAAATGGGCAGTAGCAAATCATCTTGATCGGTAGTAATGACCTGTCTCTAAATTTTCACATGCATCA
GATAATTTCTTTTTTAGTAAGTGTTATCTTACATATATGCCAAAATATCACCATTATATGGAACACTAGCTGAAAGA
AAAATTATTCAGTAGTCTTAATTTTCTAGCTAACATAAATTCTCTCCATTTTCATCATCCATTTAGATTAAAGACTT
TACTGTTAGCTGAATATTCAGAGACTTTATTCTGATTTTTAAAATTTATGAGGTTCATAATGTTAAGACTTCAAGGG
TGAGCTGTTTGTGTCATTTATAATGCGTGACTAGACAGTAACTAGAAAATGGATTGTTGACTTTACAAGATTTCTCC
CCACCACGTCCCCCCAAACCTGTGCTGCTGTGTATTTGGCCTGAAATCTTTACTTCTAGTCAATCTTTGGACCTAAA
GCCTACCAGCTTTTAGCATCCTTTAAGATTGACGTGTCTCTGGGAGACCAATAGATGCTAAACCAAATTTCGTATGC
ACTTGGCAATATAGGATAATAACAACCATACTCCCTGCAATTGTTTCCTAACACAGATGTAACAAATTACCACAAGC
TGGGTGGCTTAATAGACATTTATTCTCTCACAAATCTGGAAGCTAGGTGTCCAAAATCAAGGTCAATTATCCCTCTG
AAGGCTCTGGGGAAGAATTCTTCCTTGCCTCTTCCAGCTTCTGGTAGCCCCAGGTGTTCCTTGATTTCAAGCAGCAC
AAGTTCAACATCTGCTCCTGACCTCACATAACCCTCTTCTTTGTGTGTCTTTCTGTGTCCACTCTTTTCTTTATTAT
TATTATTATTATTATTATTATTATTATTATACTTTAAGTTTTAGGGTACATGTGCACAATGTGCAGGTTAGTTACAT
ATGTATGCATGTGCCATGCTGGTGTGCTGCACCCATTAGCTCATCATTTAGCATTAGGTATATCTCCTAATGCTATC
CCTCCCCCCCTCCCCCCACCCCACAACAGTCCCCAGAGTGTGATGTTCCCATTCCTGTGTCCATGTGTTCTCATTGT
TCAATTCCCACCTGTGAGTGAGAGTATGCAGTGTTTGGTTTTTTGTTCTTGCGATAGTTTACTGAGAATGATGATTT
CCAATTTCATCCATGTCTCTACAAAGAACATGAACTCATCATTTTTTTATGGCTGCATAGTATTCCATGGTGTATAT
GTGCCACATTTTCTTAATCCAGTCTATCATTGTTGGACATTTGGGTTGGTTCCAAGTCTTTGCTATTGTGAATAGTG
CCGCAAAAGGACACCAGTCTTTGGATTTAGAGCCCACCCTAAATTCATGGTGATGTCATTTTGAAATTCTTAACTAA
TTACATCTTCAAAGACCCTATTTCCAAATCTGGTGACATTCAAGGTTTCAGGGACATGTGACTATTCAGGGGAAACT
ATTCATCCCACCACATCCCCCTTGAAAATTCTGGAAAATGTAGTAATAAAGGCTTCTGATAAATTAGTGTGGAAAGT
ATTCACGGTTATAAATTACTAAAAAGTCTCACTGTGAGCTCTTAATCAAAAGGCCCTATAAAACATTTATTTGCTTG
ATTAAAACTACACATCCGATATTTTGGTTTTGGATTTATTATTATTTTTAGACTTGGAATAACTATTTTATGTGAAA
TAGATTCCATAACTGAAGCAGCATACCTCTCAATTTCCCAACATTTATTTTATTATTTTTTGTCTTCACACTACTTA
ATAACTGAGGAAAAATCATTTAGACCAAAGTTCACCTTGGTTGACACCATCCAGACAGCTACAGGAAATAACAATGG
AAACTAAATCTCTAAGAAAAAGAGTCTTTCATGTGAAATATTGCAGAGTTGATTCTAGATATATAGCTGTTGGAAGA
ATGGATACTATTACATAGATATGGCAGAGTGGTATCCAGCACCTTTCAACAAAGATCTTTCAGAGTCAGTCTTATTA
TGTCTGGAGAATTTACCCAGGGCTTAGGTGCTTTTACTGACAATCTAACCACCTGCACCCCACCCACCGTCTAAAGC
TAAAGTTTATTGGAAGACTTAGGAAATCAGTCTTCGGAATGTTTCTGAGACTGGTACACCCACCACTTCATTAAAGT
GCTTCACTTCACTTCATTAGACAAGAAGTAAAATACTTGTCAGGAAATTATTTATAGTACCATGTATATGGGTATCT
TATTTAATACTACTTAATGATGGTACTACAAGTTATATAAAATGGAGAAATAAGTCATCAAGTTTGACAATAATGAT
ATTTGATATTATCATTATCTTTTTTATTCGTTCCCACAGAAGTACTCTGTTATTGGTTTAGAAAAATGATATTTGAT
ATAATAAAGAAGGAAAAGGTGGTAATATTCTTTATTTTTTGTATCTTTATACCCCAGCTCTTTCACCAATCTCCCCC
ATCTCTGTAGTTCTCCTCTGGTGTCCCCAGGCAGTGAACTATTCCCAGTGGTTAGGGAACATCTCATTGAGTAAGTT
ACATCAACATTTCTTCACATTTCAGGACAACAGGAACAGTGCCAAATCCTAGCCCATTGTTCAACTCTCAAGCCTTA
TTATCCTAATAACACATCCATCCCAAGAAAGAATTCATCAAGATCAGAGAGGAATACGTATAATTTTTTATAGTACA
GTATTTAAAATGAAACAGCTTTTGGCCCGCGTGGTCTCAGTGGGCTCAAGGGGGAAATTCAGGATGCTAGCTCATCT
CACACCAAGTTTAATAAAGGGTGTCCTATAAAAAGCTAATTTCTTGCTGGTAAATTGCTTTTTAAGTAATCCTTGCT
GTTGCAAGAGACCCATTCATAGCGCTGACACTGGGAGCCATGTTGGAAAGGCTAGATATGCTCTGGGAGATAAGGTA
AGATCCAGGTGGAATCTTCTCTTTACAGAATGACAATGTATATAGCTAATATTGTCCTTTGAGGCTAGTTTGCATGC
AGTTGCTGGTATGGCACTGCTCAGCAGCCTGCTGCAGATAAGAATGAGTGATGATGCCCTAGATTTTAATGGAACTT
TTAGAGTGCATGCAGCAGTGGGGTGCAGTCTTCAGCAAAGAAAAACGAGCTGACTTGCAGGCATGAGAGATCATCAA
GAAAGATAAAGAAATAGGACATCCACTCTAGGTTAGGCAAGGCTTTTTAGAGGATATTATGGAAATGAGCAAGAACC
AATTTAATTTTTATAATGCCACTCCATTTAACTTTAAAATACAAGGTCAAGGTACTGTGTTTTTCATAATGATTAAA
GATTTGGAGCACTCTTTCTGTTGAAACATACTGCATCTGTTTGGCAGAAAAAAAAAGTGACAAAGAATAAAACTGGG
ATCAGAGAACAACAAAAACATATTCTGTCACTTGCCTAACACAAGTTAAAAAGCAAAGGAAAAAGAGACAACTCTGA
TGGACATGTTCATCCTTATCCCAACAGAAGGATTTATTTACCTAAGGTCCTATTATTTCAAGTTACTTTGATCCCAG
GATGGTAACATAAAATGTACATTTTAAAATAAAATGGAAGTATAAGATCAATAAAAACCACATATCTGTGGATAAAA
CAGCAGATTCAATCTTGTGGCTGAAAGTTTGCTTTAACCCAACATTTGGTAAACTATTCACTCTGTAATTTATTAAA
AGACATACTGTTATTATAAAACTATCTCAGTTTGCATCTTGTTGGTTCTGTCAAAATTTCATCCTGCTAATTCTCAA
CTTGTAATATCTCTGATATACATGATTAATCTATTTTAGGAATAAAACAAAAACTACCTTTATCTTACGCATTTCTA
GGAAGTGTTTTTAGATGTAAAGTAGGGGTAATTGTAGTATAGTGGAAAGGATTTTGAACTTGAAGCCAGAACATATG
TCTCTGCCAAAAACTAGGTGTGTGACCTTAAATAAGTTACTTAGCTTCCTGAATCTTAGTTTGTTTAGCTTTTTTCT
ATAAAGTGGCACACCTATCCACATCACAGTTTTGTTGTCAAAATTAAATAAAATACTATATTAGAAAGAAACTTTTA
GAAAGAAATTTATAAACTGAAATGTACTATACAAGTTTAAATCATTCTCATTATTTTCTTACCCTAAAATTTTGACC
TTATTTTTCTTAGCAAATGGCTGAATCTGTAAAATTTAACCCCCACGCAGCATCTGGATTCAAGAGAACTACGGTCA
TTTCTTTATACAGAATACTAATTATACACATATAGCAAAACACAAGTTTTTTCCAACTACTCTGTGTTTTTAAAGAT
TCAGTGTGGGCAGAAGGAATTTTATCAACTATGTTAGGGGAAAAAAGTCTGAAGAAATGAAAATAATGAGAAAAAGC
ACTGTTGATTTAAGTGCAGGAACATAAAACTTCAAGGCAAATGTGAGGCCAACTGAGTTCATATATATCCTCACAAA
ATGATTTAGTTAATTTAAAAACTTTTCTAATAAGCAACACAGGTAATCCCAAATTCTATCTTTTATAGCTCTAAGAG
TCCCCATAATTTATTCAGCAATTATTTACCACCCACTTATTATAAGAAAAGCCCTGGGATAAGTCTTGAGAAGAAAC
TAACAAAAACAAAACTTGATTGTTTGCTCTCAAAAAGCTGGGTCTAAAATAGGCAAGGTAAGATTTTGTTTTGAGGA
GCCCGTATTTTCCAGCACTGTCCATTGTAACATTAAAATAGTTTGCCAAAATCCTCACTCTGTGGGTGTATTTGCCT
AGGGTGCTAAAATTGCTTAAAAACTTTGTTATTTGGCTAACTAAAATCACTGAATAGTAAACAGTAGCATTAGAGAT
GGCAGAGACATTAGGTGTCATGCAGTTCAACTGCTTCACCTAGCAGACAAAGACATTAAGTTCCATTTCTTAAATTT
AACTATCTGGTTGAGGATACACAGTAGCAGAGCTAAATCAAGAACCTCTTGGGGTTAGAGTTTTTGTTTATGCATTA
CTTTGTTTTGGAATTAAAAACAGTGCCTGTTTGCTAAGTTAAATTGAAAATATGCTCTGAAGGAGAAAAACAGCTAT
AAAAATAGACTTAACTTCCAAACTATGGATCACAATAAACTAAAGAAATAATTTCTGTAGCAATAAACTCCAACACT
TTCCATAGGACCAGAAAGGCTTGAGAAAGAGGAGAACAAAAAAATGCTTTGGGGCTTACCATATATATGGAGAAAGC
TAAATGAATAAACCAGTTGAAAGACAGCGAGTTATACTAGTAACAATATTACTGATATCGGAGCTCTCACTTATAAA
TTGTATATTATGATCATAGTGACTAGGTACTTTATATCTGCTTTCTCATTCCTTCCTCACATTAATTCACATGTAGG
ACAGATTACCTCTTCTGTTTCTATCCAGAGGCCTAGAGCTCAGGCCCTCATCGAAGACAGACAGAGCTATCATCCTT
ATTCTAAAAAAAAACTAAGACCCCAGACATAGCTGTGCTACTTATAGACTAGAATGTGAGAGAAAAAGACAAGCTTT
CATCATGGGCTTAACAAACTGAAACACTTCTTCAATTTTGAGATTGAGAAACTTAGCTAATGCTAGGTGTAAAGATG
ATATGCTACCTTCATAACCTTGGTGAGGAGAAATTAGCATTTCTCTCAGTCCTAGAAGGAGGATGACCATGAAGGTC
TTCATTCTCTTGAGAAGATAATCAAATGCTTCACTGCCCTGTTAACGGTTTACTCAATATTCACCAAGAAAAGTAGA
TGGGATTATTTTTGCAGACACTTATACGGGTAATTTATTCTGATAAGCAGAGACATACCTTTAGTGCATAAATTGTT
CCCTTTGTGCTCTTTGTAATAAACATCACCATAGAGAACAAACACGAAGTAATGACATTGAATTAAAAGACACCATA
GAGGCAACAGCGACTGGAATTTGTGAAAGTAAAAGGATAGTGCAAACAGTTGTGCGTTGCATTCTGCTCTGAAGATT
AACAAGCTGGGTCAGGCTTTGACCATCATGATGAGCAGGAGATTTTTCTAATGGAAATCCCCAATCAAGTTCCTGCT
GCACCCAGAAAGGAACGGCTTACAGAAATCTTACATTTCTTTGCACATACCAAATTGCTTGGCATATTCTATCACAA
GGTTTACTTTCCAGGGAATGTGATCAAGAAATCATGATCCTAATTCCTAGTTAACCCTCAAAGTTTCTCAGAACAGT
CAGTGCATCACTGTCAACTTTTGTGCAATGTGGAAATCAGAATTGGTCACACGTTTTTCCGGCCACTGTTTTAGATT
CATATAATATTAGTGAAATCATGTCAGACTGGTATAGCCATGAATTTATACTTCATGAATAGGCACTCAATAAATAG
TGGATTAAATCGACCGATTTGATTTTTACCTCCAATAATTTCAAAAATATCATTGAAGACAAGGTTGTTGAAGCTGT
CACTTTTCTTGCTGAACCTTTGTTGTGCCAGGAGGAACAGATGGTAAAATCAAAAGTGATTAGAGAATCAGTGGGGT
GGGGGTGAGATTGGAGGGGAGAGGTCTTCCCAGTGAGACCCGCTAGCGTCTTCCCTGAGCAGTATGTTAACCCAAGA
CAATTTTAGAAATCTGTGCCCCTAAGTTGCTTGACATCCAAAGCACACTTGATGCATCCTACATTTCTAAATATTTT
TATTGTTGTTTCTCGGTAGTAATCATCTGGTTTAGTCACTCTAAAAGTCAAGGATGAAATTTTAAAATGCAAATAAA
AGTGCCTACTTTCTCTCTTTCCAATTCCTTTTTGTTTTATTGAGGTATAATTTACATGCACAAAAAAATCGCCTTTT
TAAAGTGTACAGTTTGATGAGTTTTGACAAACATATGCAGTCCTACAACCACGTCCGTGATCAGAATAGGAAATATT
TTTATCACTTCAAAAAGTTTCCTTGTACTCCCGTTGCAGTCAGTCTCCTGCCCCACCCCAGCCCCTGGAAACCACTG
ATAGGTAAAAGCACTTTTAATCTGAAAGGTATTTAATGTATGGCAGTGTCAGTGGTAATAATAACAAGATTTATTCA
TTGGTTCACTGTATTTTTGAGCACTTATATGTGCCCGTTGTATGCAACCCATTATGCTCAACCCCTGCCCTCCTCAC
CAGGGATAAACTAGTGGCAGAGATAGACAAAGAAGCCGTCTCTCTATCACCCCTATCTTATAGAACATTCTTCAATG
TTAGAAATGCAGTATAATGTGGCCATTGAGAACTTGAAATGTGCTTAGTGGGAATGAAGAACTGAAGTTTTAACTTT
ATTTAATTTCAATTAATTTAAATTTATATAGCCACATGTGGCTAATGACTATCCCACTGGAAAGTACAGCTTCTATA
CAATATGATAATATGATACATTATAACGCAGGAGTTTAACCAAGTGCTAAAGCTTTACTATCACCAGGGTCACTGGT
GTTATGTGAAAAGAAAACTTACAATAGAAAAATAAATCCTTTAAATAGTCACAGACCTGAGAAAGTTTCCTTCTCAA
GGGAACACACATTGGCTCATTCAAAGGAGGTTAAAAACTAGCATTTAAGGTAATTTCATGAAGCTTTCCTTTGGATT
TCTCATGCTTATTGTATACATAAATAGGCAATTTTCGATGGGACCTAATAAATCACTGTTTTTTATTTGAACATTTT
AACAAAATTATCAAACAGCATTGCATTTATGTTCAACCTATTTGTTCTGAGAAAGACAACGATTAAGTAGAAGTCAT
CAAAGTTACCAGAACAATTTTTGTTCTTATGTTTTAGAAGGCATTGAAGGTGTTTAAAATGTACACTTATAGAGTCA
GAGTACTATGCAACTGTGGCCCTTATAGTTTATCCGTCATGCATCTAAAGCCATTGTTACATCTGTTTCTAATTGTG
CATGGATTGTCCAAGATACACAATTGGAAATTCCATTTTATTTATCAATTTGAAGAGGTTTCACCCATGTGGTCACT
ATGATCACTATGGAGTCACATTAAATTGAGAAGTCTCCAGAAGTTGCAGTATTTATTTAAAATTCTAACTTTCTTCA
GAGGAACAAATTCTCCATTTCTGGATTCTGAATCCTCATTAGCCATAAGGTTGTTGTAAGAATTTGCAGCTAATAGG
AACACATCCTGGGGAGAGACCAGTTGAAAAGTAACTTGGTTCTGAGTGAAATTATACAGAGACAGTTTCTACTTCAG
GTGGTGTTGCTAATGAAGCTATCATGGTAATTTTAGCCCATATGATCCCTAAACGACTTCAGAACCACTTTTCATCC
ACTAAGAACCCACTTCAACCACTGCCACGTTCACTACCACAGTATAATATGGAACACCCTCTGGAATTCAGTAAGTA
ACTTCTTAACTCATTGGCTATAGAGCTTTGCCTTTGTAAATTCTTTCCTTTTGCAGTAAAAGAGATTGTTTCAAAGT
AATCCAATTAGTCCCTAGGCATGTCTAGAAAGGTAGAGTCAACAACAGTAAGGTAATAGTCCTTATAAGATATGTAA
GAAATTATCAGTCATTTACTTTAAAATAATTTGTACACTTTTCCTTTTATATGGTTCTTCTATGTTGAAGCCAGTGG
TCATCCAGTGATTAAGATTAGCCAAACTCAAAAGGCTAAAACTAAATTCAAATGGTATTATTTTGCTTTAATTTTAT
GCAATGCTATGTATTTAAATTTCATGAAAGTTTCGTATGGCATTGCTATCAATTTCAGTCAGGATAAATTTCCCGTG
AAATAATCCACAATTTTCAACTGTACGTTGGGTACAGGTAAGGAAACACCCTTAAGAGCTTATCCAGTTATTAGCTG
GTATTATAAATTTCAAGTAATTCAATGTTCAATTAATAAACAGTTACTTTAAATGGGAAAGTATGAGTCAAGAGTTA
GTACAAAGGAGAATCTTAAAAGATGAACATCAAAGAATCTTACTATTGATTTGTTGGTGCCTTTGCTTGCACTTCTC
CAAATTGACTTGACGTTTTAAATTTGTACTGATAATCATCAGAGTCAAATCTGCTTTTAGGCAAAAAGTATCCGCTA
GTTATTCCCCTACTATGAAAGTGATGAGATGAATTGATCATGTCTCCAGTGTATGGATGGATGTCTTTGAGGAAGAC
CTACTGACCTTATGTTTATCTTCTGTCAGCATGGTGTGACTATGTGGAGAGACAGTGCTATTTGCTAAATACTTTGT
TTTTCAAATAAAAAGATTTCACAGATTATGCATTGTAGAATTTATAAGTATTCTTTTATGTCTTTGAATGTGCCAAT
ACAATTTTTATGAAGTTGGAACTATTTTATCTATTTTAATGAAATTGTAAGCCTTCTGTGAATTCTTTTATTAATTT
TATTCTGAAGAAAATCTGACCAGGTTAGGGAAATCAGGTCAGGTTACGACGTGATCCCAGTGGAAAAGCTGAACTGT
GGACTGTGATTTAAAATAGGGAAGAGGTACTGAAGTGTTGTTTTTATTTTTGTTTACAAATCAGCCTTTCTAACTAT
TATGTACTCCCATCCTTCTATCTTTTTCTCCACCAGAACGTATTAACAGGCATGCATATAATTAATGCTTTTCTTGA
GATAATATTAAAATTAACTTCATCTGTCAGGCCGTCTGGGCTAAAAGTACACAGTCAGATCTGGGTAACATTTGAGT
TGATGTAAATATGCCCACACATACTGACAATGCTTACCATTTATTGTGTGAATGAAAAGCAGTGTAAATATTGTTTG
TTCTACTAGGGAAGCTCCACATTTTAATCAAACTTTGACCGTATTTCTAAAATGCCAGAGCATCTGGAATTGTTAAA
GGAACTGATAGTTTTTGTGTTTTTAACTGTTAGGATACTTGAAATCCAAAGGGTAAAGAAACTCAGCTGATTTATAC
GTTTCTTCCTCTTTATTTTAATGTGATAAAATGTAGTTTTTGTCATGGGCTGACAAACAGTGGTAGACTACACTAAC
TCTGCGTTTGCTGGGTTTAATCTTACCCTCTCAAGGCATGGAATGGGAGCTCACTTCAGACCCAGCCATGCTTCACT
GTCCACTGCCTTCTCATGGATATAGTGTGAACATTAATTAGATGAATTCCATAAAGTGCTTTAAGCTCTTTGGAGAA
AGATACTCGCTGCATAATTATTCTTAACTCCCATACGCTCTTATGATATAAACCATTCTGCCAGGAAATCCTTTTTA
GGGATTATCACTTAAAATGAAATTTTCATTATTAAAAGCAGGAAGAATATACATCTACTGACAGACGAAAATGTGCT
TAAGGCGACTGCTTTTAAATAGGCAGAAATCCTGAACTATGGAGCCATCCATGCCTGAAAATACTGAGTAATAATGA
AAACTGGTAGCAAATTTGGAATATTAATCATCACATTAAGTTGCAAAGAAAAAAAAATACAAGCCACATGCCCTTTA
AAAATACGTGCACAAATCTTTATTCTAGAAATATATAACTTTAGGCCTAAAAAAGTACAAAAAGTAAATTATTTTAT
GGCTCTGAAAGTATCCTTAATTTACTCAGGTGACAACAATTAGTGTTTAAAGAGTTAGTTTTCAATCTTAGCTACAA
GTTGGAATTACTCTGGAAGCTCTAAAAAAACAAAAAACAAAAAAAAATAGAGATGCCTAGTTCCCACCTGCAGAAAT
TCTGATTTGATTTTTCTGGTGCGAGACCTGAGAATAGGAATTTTTTTAAAGCTTCCCTAGTGATTCTAGTGTGCCAC
CTAGGTTGCCTTAAGGTAAACCTCATATTATGCAGAACCTAGCAATCACCTATCCTGATTTTATAGACGAAGATCAT
AAGACCCAAGAGGGCAAATTGATTTATTCAAGATTGAATATACAAATGATAGAAGATTCACATAAGATGCAGTATAC
AGAGTGGCTTGTGGATTCTTGCCAATGCAGGCAGCAGAATTTTCTTTAGGGTTCACCCAGTTCAGGCACCTCTTTGC
AGCAGCACTTGACTAAGGTTCTTCTGATTGGATCATTATATGGGCAAAAAGAAAAAGCTTAATTGAAAAGAGCTGAA
CCCACATTGTGGAATGGAAGATATACAGTTTACACGTTATAAATGATTAATATTCATGAAAGCATACTGCCCTTTCC
TCTTCCCTTCCCATAGATGACATCATTGCATTGGTGTAGTTAGGTTGGTGGTTTCTTGTTGTTGATCTTGGTTCTGA
CACAGTTCATCACTTATTATCCTGGCTTATTATCTACTTCTACATTCATTGTTCACTCACTCACTAATTAATTCAAC
ATGGTTTTTATTGTTTTGGACCGGTTATATGCCTGCAACGCTACGTAAGGCTGAGGATATTACAATGAACAGGAAAC
AACCCTGAAGTTTAAGGTATCAAGCCTTTGAGTTACTGTCTTTTATCATAGCTGATATAAAATTGAAGCCCCACTTT
TTTTGTTTTCAATTACTGAAAATTCAGTGCTAAAAAAATGTGGATTTTTATTCAACTAGATAAAGTACTACAATTAG
GTTTCCACTGACCTTGGCTGTTTTTGTTCCCAGTTGCCATTACATAAATCTGTGCCACTCACAACTTAGGAAGGGTG
TAACATTCTCTGTAATAGTTTGCCTTTCGAATAGTGTTTGGATTCATTACTGTCCCTCGCAGTTTGGAATAATGACC
ACTGAATAATCAGTGTTTGGAGACTAAATTAGTGCTGCAAAATTCCCTCAAATTACCTACTGTTCTTTTCCCTGTCG
ATGTATCCTCATATTCACTATGATTACCCTGAGAAGAAAGATATTGTTGAGAACCACTTTACCTACTCGAAGTTTTG
GTATTTCAAAGATTCATACTTATGTCATGTTGATTACATTAGCACTAATACTATTGGCAGAATTCTAATTCACGTTA
TTTTCTTTTTTTCCAATTTCTCTCCATGCCTATGTGTTGTCCCTTCGCAGCTATAAAGCCATGGCCGATTCATGGGT
GCTTTTGTTAAGGCGTTCAGCAGTCACGTTTGTAGATTTTTGAATGGGACTTAGAGCCCTTTTTTGTTCTTTATGTA
TTTCTCTATTTCTCAGCAAAGGAAATGCAGACATGCAAGAAATAGTGATCAAATGTCCTGTGTACTATTGTGGGTGT
CATTAATGGTATAGGGAGAAATAGAAAATAGTTGCAAAGATGCATTTAACAAATAAACGAGGTCTTGAGATTCACCA
TGAATGTGGCCCCTTCTATGAAAAGTAGTTAACATCCAACTGCAAAGTTGTACTGGATCAGTTTGACTTTAACCTTT
AGCTAATATGAAAATATGGAATTGTGTGGTGGTGCTCACAAAAAAGAAAACTCATTTTTCTTAATTATCATCAATTA
ACATGTACTGACTACCCATGAGGGAAAGTTAATTTGCTCTTGAGTGGAACCAGTTATTTGCCCTATTATTTCTCCCT
TGCTTATTCCCCTCTCCCTCCCTCCTCCCTTTCCATTCAACAAAGAAAAATAGATAAAGCAATTTCTGATTAGCCAG
TGAAAGCCTCTAACATAAAATTTCCAAAGATGTGCCATAAATTATCCACAAAATGTAAAACTTTTCAATTTTGGTTT
GCATTTTCTTTTTTCTTATTATAAAGGTAATAAGTGCTCATTATAGAATTTGAAAAATATAGGAAGTTGCACGGAAG
ACGAATAAAATCAGCCATAATCCTACAAACCTATTGACACTTGTACATATGTTTGTTATCTCTAATGCATTCATTAT
GATAATGCATCTTTTCAACCAATAGAGTAATCACTGGTGACTTTCAAATTTGCCTACTCATTTTTCACTCTGTGGAC
TTACTTTACTACCTCTTGCCCTTTTTCAGTAAATGAATAAATATTTAAGTAAGTAAATACAAATGTAATAACTTATG
CGCTCAAGCACACAGATACACACAGAGAGAATTTGGAACTTCGGAAATGCCATCCTCTCCCTAGGGCCGCAAGTGAG
TTGATAAGCACGTAAGGAAGGATAATCAGGGGAGCCTTCTCGTATTGCCCAGATGGCTCAAAATTCGTCATCTCTAC
CAAACAACTATTTGGAGCTTTGAAGAAATATCCATGACCCCTTTGAATTCTTCAGTTTCTTTCGCGTTCACTTTGAG
AACCAAGTGACAAGTGAATTTCCTGACTTGGTCTTTTAAACCTGTTAGCGCAGTTCCATTGAGATTTTGTGGGCACA
AGATTGCAATGAAGAGATCAACAGGGAGAAATTCATTTCCCTATATATGTGCGATTAATCCGGAGTGCTAAGGGCAG
ATATAAAGCAGGTGCCTACTCCTGTATAACTTGGAATAAAACCATTTCCAAAGGCTGATGATCCTCAAGTCTTGTTC
TGCAAATGACTGATGTATAACTTCAGGCCAATTTTTCTCCAGTTAGTCTGTGTCACTGGGAGTCCCATTTCTCGGGG
AGCAGCCCCATGCTTTGTCAGGTGCGGAGCCCACAGAAGGTTAATGCGAAAAGAAGGCCTCTTGCCAGACTGTTTTC
CAGATGATACGTAGGGTTATTAGTTTGAGCTCCTTAAGAAGATTTTTCTCACCTGTCCTACCAACTTATGTTTATTT
CATTGGTGTTAGAGGGTTTCAGTGGCGGAAGTAAAATATTTAGCGGGGAAGGGACAGCGTTCATGGGAATTTTGCCT
AACTTAATTTTGTATCTTTAGCTCATTCGTAGTCATTGTACTTTGTGTTTTGTCAACTGAATTTTGTTTGCATACAA
AGGCACAAAATGTTTGCTTCAGACCTGTCACTCTTATTTTTAGCATGGTTAGACAAAAACTGAGATGCTTTAATTGT
CTAACTTATCCCAGTTTAAGTGCTGCAAAATCTCCCAGGCAATGTCATGGGCAACTAAGGGATAAAATCAGAGATTT
AAAGGTGCCAGGTTTCCCACGCTTCTAACAGTTGGCGTTTTGGGTGTATACAATCCCTCAGCTTTCTTCTTTAGTTT
ATGGAGTCTTGTGGAGGGAATAGCAGGTTTTTAGCTAAAATTATCATGCTGTCGAGTTGGGTCTCTAGTGCATCCTG
AAGAGCTTGCATTATTTACAGAGGCTGGGCTATCATTTTAAATCCTGATGCTTCAATGCCCGTTATCATTCTTGACA
AACTCTTCCAGCCCGTGGTCTGTTTTCCTCTGTTTGCTTCCATTTACTTTCCTGAGCAACCAGCTGAGCAAAGATTT
ACATAACTTTTGTTTAAACAAACCCTGTACAGTTCACTCTTTCAGCCAGTATGTAAACACTTTTGAGACACAGTTAC
ATTTTTCTATTTTAGTCCCAGATTCTGTTTATTTGCTACATTTTTTGTGCCCACATTTTTGTCTTTGTTAAGTCTCT
TACAGATTCACATGAAAAACCAGAAACCGTGGCTGCTCAAAAGTCATTAATAATGAGATTTTTAGCTACTGTTTCTG
CTTGTAAATTCTTCATTTCACATAATACAGTCTCAAAAGGCCACAGAGAATTCAGCCTCGCTTATCTCTGTGTTGCA
GATGATGGCTTCTAGCCTTACCCAATCCCAGTGCAGCTTGCTTGCCATCCAGGAGTCGAATTTGTTTCCATCTGACA
TTAGCGTATTAAAAAGATTGGAGATCAACAAGCAACAATGTTCTTGTAGAAAGGTAATCAAGGTTTAGAGCCTGTGT
GTCATGAGACTCCTAGCATTTGAAACCGCTAAGGGGTTGACCACCATTGTCCCAAGCACCTGTTTAAGATTCTTTCC
TATGATAAGGGACCTAAAGTGATTAGCATACTGATAAGATTTTCCTAGAATAACCTATTTATTTCAGTATTATTCTT
TCAAATCTTAATTACCATCTTTTCCTTTACCCAGGGTCTTCTTTCTACCTCTACGACACATTTAATTACCTATATTC
CCCAACCTGTACCATATTAAATTTTGAATGGAAGTTTTATAGGGTAATTTATTGGAAGGATGGCCTTGAGTGTCATT
ATGTTCAATGAATGCCCTATTTTGACAAAGAGATGACTAAATGTTATTGAAATCTTTTTAATCCACCACGCTTCTGC
TTAGATGTAAATGCAAATCTGTTCTTTACATTTGTGATTGAATTGAACTTGAAAAGTACCGCCATATTGATTCCTTC
TGCAAATAAAATATAATTACATTTCCCTAAACTTTCTACACTCTCCCAAGAGATTGGCTGGCTTTGTATTGTAGATT
TTTGGTGATCACAGAGGACAATGCATTATCATAAGACCAATAAGATTTATTTTTACCTTGGTAAAGAATTTTAATTT
ATTTCTAGTTTCATTTTCATTTATATCCATCTCTTCTCACCCTCTGCTCTACAAAAGTATATATGACTATATAAATT
GAAAAAAATATCAAGTGCAAAATTACAGAAATAAATAATTAGGTTATTTTAGTGGAGGAAGGTTTGTTGTGGGTGGA
GGAGGAGAGGAGTGAGCCAAGAAAAACGAGGGACCATACGTGATCATATTTTTGCAGCTATTTTAAATTGTTTGTGT
ATATACTTTAAAATATTATAAAATAAAATTTTAAGTGCAATGCATATTTGGAGCCAATGATGAGGGATAACTTCAGA
AACGTAGCATCATCATCTAGTGCTTTCATAGTCCTTTCAACATTTCCAGATAGTTTTAATGGCCTGCTCATGGAGGC
AATGCCCTAATTTTAACATATCTCTTCACAACTCTGATTTCTTGCTTCCTAACATTAAATGTCTTCAAAGCTTCTTT
CACCACTAATTCCTTATCAAGAGGATAAGCCAGTTTATTCTTTAAGAAAAACTAGCTACACAAAACCGTAAGTCATT
CCAACATAAATCCTTCACTATCCTCTCTCTATAGATTTGGTTTTGATTCCTCCTGCTGAAATTCAACCTTCTTTCTT
CAGCTATCCACACGTCTTACCCTCTAACTTCCCTCAGGAGTGTCTATTAGCTCCCATTACAGTGACCACAGTAATAT
AGTAATCCCCTGCTGTTCTCACTCTCCACTTCCTTACACTGCGTTTTAAGTCTCTTCATATTCTTTATCACCTTGTA
TCATGCATCGGTTTTCTTAGTTGTTTATTTTATGTTGCCTTCATAAATTCCATGAGAGCTCACTGCCGTATCTTTAG
AACATGGAACAGTGCTTGGAACATAATGGGCATTCCTTAAATAGCTGTAGAATAAACTTTCAAAATCAACAATAATG
TATTTGCCAAATCCATTGGCTTCTCTGCCATTTTATCTTGTTCAATACCACTGCGATATTCCCCTTCCTTTTTTTTT
TTTTTTAAAGTCTGTAACCCTTTAGCTTCTGTAATATTCCTAGTTTTTTATTCCTCTCATGTGTCAAAATCATCAGT
TGAGGCTTATTGTTTTCTCTTTCTCACTCTGACCTCACCTTTGTTTACATCTCATCTTCTGGCTTTGGCTATCCTGT
TTTTTATCTCTGTTCCAACCTGTATTTCTAGCCCTACTACCTGGACATGACATGTGGATATCTCCGTATGACCGCAG
TTTCCATATGACTTTGCAAATTCATCCCTGCTCTCCCCTCCAAAGTCATCCCCACAATTGACTTCCTGTTCCTTCCA
ACCTATTAAGGTTCAAACCCACTTTTGCTCCTCCTTTGCAGGCTACACTTTTCCTTCTCAGTACCTCTTTTTTTTCC
AAGTTCTTAGATAAAAGTCATAGTACCTTACGTTGTAATTGCCACTGGTCTGGTCTTTCTGCCTGCTTTCCTTTCCA
TTTGTAATCACATTATCCATTCCAATCCATTTATAATACTGTGATCAGCCATAAAAATAACATTTATCATATCGTTT
GTCTCCTTAAAACCTGTAGTAGATCCCCTCTATTTACAAGATCTGGTATAAAATCACCCTTCCTGATATTCAATGCC
TGTTTTAATATAATCTCAATATTATGCGTCATAAATCCCCCTGTGTTCTTGCACTTTTTATTTCTTATACATCTCAT
CAACCATGTCTTATCAACTCTCAAAACCTGTATTGGTTTTCAGGAAAACTCATAAATTATTCTTTTGTAGACCTTTT
GTTTGTCATCTTTGAAGATCTCTCTCTGAACTACAATATTTTGTCTGTATAATCAATTTGGAAATTCATCAGGTATT
GAAATATGACATGTCTTCTATTGTCTTGAACATTAATTAAAACTTTATTTGACTTTTTATATGCTTACATCTTGTTT
CCTCACGGAGTGTTAACCTACTAGAAAGTAATAGTTTAATCTTATATTTATTTTAATTCAGATTTAGTAGCATACTT
TACACGTGGTAGGATGTGTAACTGCCTTACACCTTGCTTACGTGAGTTATTAATGTTTTCGTATATTTAATCTGAGG
ATGTACTAGCAATGTTAAAACTGTACCGCATGAAATTGAGTAATTGAACTATTTGTTTTAAATGTGTTGCTTAACTT
ATTGTACCATTTTCTCATAATCACAGCTCAAGTTAACTTTGTGGTTGTACGTATTATTTCTTGTGAAATGCCAACAA
ACTTAGAGCAAGGAAAATAACAGGTATAATCATACTATAAAGGCAACCTTAACACTAGCATAGTCTCTTAGCTCATA
TGGTAACTACAATAATGTACAGTGACAAAGAGAATATTGTACTTTCTTAGCACACACTTTCCTACTACTCTACTGTT
GTGGATAAAAACAGACATACTTTAGGAGAAACTATGTTATTTCCAAATAATGCCTTAAAGGTTACTCCAGGAAAAGG
CATTTACATAAACTATCTAGGAAAAGAACCTTTTAAATAATATAAAGAGCTCACCCAAAAGGACTGAAGTGTTTAGT
TGAAAAAAAGTAAAAATGTCGAAGACTTTGAAAAATAGTTTCTTGCAGTATATTTTCATCGCTTCCACTTACGTTAT
GAAGACATTAAGCGCTAGTTTATCAAAAACTATTTTTGTACATGTCTTCTAATGACAGAACAATGTCAACATGATTT
TCATCATTGAGAATGCGTAAAGAAACCCTTTGTACAGTTTTTTCTATGAATGTTCCCCTAAGATTAAAGCAAATTTC
CAACACGAATTAGGCACTCCGAAAGGAGGAGGGGAGGGAGGGGAGCAAGTGCTGCAAAACTTCCTGTTGGGTACTAT
GTTCACTATCTGGGTGATGGAATCAACAGAAGCCCAAACCTCAGCATCACGCAGTATACCCTTGTAACAAACCAACA
CATGTACCCCTGAGTCTACATTAAAAATAGAGATTAAAAAAAGGAAATCAGTATATAATCTAATAAATACCTCTCAA
GCTTTCTCATTTTTAAAATAAAATTTTAGATTATTATTTTAGGAATAAAATAGGCTCTTCATTGTATATAAGTTCAT
TTCTGAGTTGCAAAAATCCTCTCTTTATGTTTTTTTCCCCGTATTAGCATGTTTTTCTCCTGTTTTTCCCCACTCAA
CTTGGCTGCCACAATCAGAAAGCACAAAGACAATTTTTTCTTGCGCTTGTAAATCAAAACCTTAGCATCAGACAAAA
TAACTGCTCCAGGTCTGTCAAATAGATTCATTTGAGCTTTCTTCATGCATTGAATACGGCAGAATTTCTGACCTGAA
GAAATCTAGCCTTTTCCAAATTTGCTTTAAGAACATTTTGCAATAAATTTAATATAATAAAAGGAAAAAACACATCA
GGCTAGAATTTGGAACCGATTGTTATTAAAAATCTCAAGTCTATCAATTTAACTTCAACAAATTACTTAATTTCTGT
GATGGTTAATTTCATGTGTCAACTTGGCTGGGCCGCAGGGTACCGAGACATTTGGTCAAACATTATTCTGGGTGTGT
TTATGAGGCTGTTTCTGGAGAGATTCACATTTGAATCAGTAGAGGGAGCAAAGCCGATTGTTCTCCCTTGTGTGGGT
GGGTCTGATCCAATCAATTGAGGACCTAAGTCCAATCGATTGAAGACCTAATCAAAAAGCCTGATTAAAAGGAACTC
CTGCCTGATAGCTAAAGCTGGAACACCCATCTTTTCCTGCCTTTGAGCTTGAATTGAAACCTTGGGTCTTCTTGAGT
CTTAAGCCTCCAGTTCTGGGGCTGGAACTTAACGTCATTGGCTTTCTTGGTTCTCATGCCTTTGGACTCAGACAGGA
ACTACATCATTGGCTTTCCTGGGTCTCCAGCTTGCTGACTGTAAATCTTGGGACTTCTCCAGATTCGTAATGAGCCA
ATTTATTACAATAAGTCTCTCCCTCTCTGGTTTCGAGAGAGAGAGAGAGAGAGACAGAGAGAGAAATGAGAGCACAA
GAACGTGAGTGTGAGAGTGCCCTAATATAATTTCTCTAAATATCACTGGTTACTCTTCAAAGTTATAAAATTGGTAT
AAAAGGTGACCTCAATTTTTCATGGAGTTAATGTATGAAAGTCACAATTAAAAAGGAAGAATTAGTTCTGGTGTCCT
GAAAGTTATTTGAATAAATTAATATGCTATGGAGGCTTTAAAATACTATGAAAATTTAATATTGTATTATTCTTAGT
GTTGCTATTTTTAAATAGCACTTTTTCTTTTCCTTTTTTTTTTTTTTTTTTTTTTTTTTTGAGATGGAGTCTCACTC
TGTTGCCCAGGCTGGAGTGCAGTGGCATGATCTCGGCTCACTGCAAGCTCCACTGCCCGGGTTCACGCCATTCTCCT
GCCTCAGCCTCCCAAGTAGCTGGGACTACAGGCGGCCGCCACCACGTCCGGGTAATTTTTTGTATTTTTTTAGTAGA
GACGGAGTTTCACCGTGTTAGCCAGGTTGTTCTCGATCTCCTGACCTCATGATCCACCCACCTTGGCCTCCCAAAGT
GCTGGGATTACAGGCATGAGCCACCATGCCCGGCTTAAATAGCACTTTTTCTTGTGAGTCACTTTTTAAATATTTGT
GCAAACCTTGTTGCCATTCTACTCAAGCTAATATCCTAAACCGAGGACATTATAACATTTCAGGAGTCAAAACTTCA
GACACTTAACATAGTATCCTCAGGTTCATCCATGTTGTCATAAATGACAGGATTTTATTCTTTTATATGACTCAATA
ATATCCCATTGCATATATATGCAATATTTTCTTTATTCATCCATTATTAAACACTTAAGTTGATTCTATATCTTGGC
TATTGTGAATAATGCTGCAATAAACATGGGAATGCAGATATCTCTATGACATACTGATTTTATTTGCTTTGTCTCTG
TCCCCAGTAGTGGAATTGCTGTATCGTATGGTAGTTCTATTTTTAAGTTTTCGAGGAACCTCCATACCGTCCTCCAT
AATGGATGTACTCATTTACATTCCCACCAACAGTGCATAAGGGTTCCCTTTTCTCCATATTCTTGCCAACACTTTTT
ATCTTTTGTATTTTGATAATAGCCATTCTAACTGGAATGAGATGATATCTCATTGTGGTTTTGATTTGCATTTTCCT
GATAGTGATGTTGAACATTTTTTCATATGTTGTATTAACTAAGCCAAACACAGAAAGACAAATGCAGCTTGTTCTCA
TTCATATGCACAATCTAAAAACATCGATCTCATAGAAGCAGTAAATGGACGGTGGTCACCAAAGAATGGGGGAAGTA
GGGGAAAAGCGAGAATGGGGAGAGGATTGTCAATGGGTACAAAGTCACGATTAGAAAGGAAGAATTAGTTCTGGTGT
CCTGTTGCATAGTATGGAGACTATTGTCAACAGTAAGGTATTGCGTATCTCAAAACGGCTAGAAGAGAGGGTTTTGA
AGGTTTCTACCCCAAATAAATGGTAAATGTTTGAGGTGATATGCTAATTTTCTTGATTTGATCAAGTAAAGGTCTTA
ATTGTTTGGCAATTAAGACTCATGAATACAAATAAAGGTCTTAATTATTTGGCAAAGCATGCTGAGTTTTGTAAACA
ATTCAGTAGTGATTTTTGAGAATAGGTCAATAGCAAATATTAATTAAAATGTCTTCTATTTATGACCTACAGCTAGA
TGGTAAACAGATAGATGATAGATAGATAACTGATAGATAACTAATAGATGACAGATAAATGATAAATAGATAAATAT
AGATAATCGAGAGAGAATACCTTTCCCTTCACACACGTGCATATAGGCACACTCCATTTCTATCATAGTTACCAGGA
TTCAGACATTTTGTCTCACTATTTTTCTCAATGTGAACATGCATATAGGAATATTATAGTTTTTGTTCTGTGCCCAT
TTTAGTTCGTTTTTTAATATTTCAGGACAAAGGCAATATGGCGGTTTCACTTTGTTTTTCATTTTTGCTTATACTTT
TTAAAGCTCAGTGTAGAAAAGTTTGAAAATACACAAAAGTATTAAATTAAGACAGCTGGGCACAGTGGCTCACGCCT
GTAATCCCAGCACTTCGGGAGGCCAAGGTGGGTGGATCACGAGGTCAAGAGATCGACACCATCCTGGCCAACATGGT
GAATCCCGTCTCTACTAAAAATACAAAAATTAGCTGAGCATGGTGGTGTGTGCCTGTAGTCCCAGCTACTCGGGAGG
CTGAGGCAGGAGAATCGCTTGAACCCGGGAGGCAGAGGTTGCAGTGAGCCGGGATCACACCACTGTATTCCAGCCTG
GTGACAGAGCGAGACTCTGTCTCAGAAAAAAAACAAAACAAACAAACAAAAAAGCACCTATAGTCTTTCTCCCATAG
GTTGCCTTCTTAATGGGTTTTACACCTTTTGATGTTTTCTTGAGTTCTGTCCCATTAGCAAGTAGTATTGTACAAAA
AAAATTTTATCATCTTTTATTTAATATTTTATTGATGTTTAATAATTAGAATTATTTTAAATTTTATATGTCATTTT
AAAATGCAATACAATATAGTAAACTCCCAGATGTGATTGTAAATAATTAATTATTCTCCCATTATTGGGCATTGGGA
CTGCTTCCACATTTTGGTCACTGCAGTGAACATCCTTGTACATGAATCTGTATGTTGAAGTTGATTTCATTCCACAC
TCCCCTTCATTCAAGGGGCTCCAACCATTCTCGTTTTCTTTCAGCTTCTTTATATCCAGGCATATAAAGTTCCTTCC
TGACTCGGGAGCGTCATACATGCTGTTTTCTCCATCTGGATAAGTAGTTAATTCTGTTCTTCTTTGTGCATCTCCCG
TTTCAGTAACTTCATCTCCAAAGCCTTTCCAGGTCACTTTATCTAAAGTTACACCATAATCTTGCAAATCCTCAACT
ATTGAGCATTATTAGTCTCCGTTATCATTATTCTCCATTATTCTCTGTGAAAGCATCCCGTGATTTTCTTTTGTCCC
TATTACCACAATATGTGTTTATTCCGTGTATGTACATCTTTGTTTGTTTATTGTTTGTCTATACCTGCAATGAAATG
CCTAAGGTCAGGAACTGTCTGATGCAGGATGCAATGCGCTCAATAAATATTTACTGAACAAATTAATTCATTTGCTC
AGTCTTGCAGGCAAATGGTACTTCTGTATATTTAAATATCTAAAATGAAAGCGTTACTCGTTACTGTTGGTTGTCAA
TCAAAATTTAAATGTCGATGTTTAAGCGTGAAAGACCTCTGTCAAGTTAATCTGTACTTACCCAAAGGCTATTATGT
AGAAGCGACATAAATATTTTCCTAAATGTTGATTTTCATATTTTAAGAAGACAATGAATGTTTCAAAGCATTTTCTT
CTACACAGCTATTTATTCTGGAGAGTGGGGCATATGTTTCTTAATATTGTTAAAATTGGCAAGGGGATACTGTTGCT
ATATACAAAGAACACCTAATCATCATGCAGACGTTTTGTTTCTGGCTCTCAGTTATGAAAAGCAGAGATTTTAAAAA
GTTACCTTTATATGCTAAATTAGGAATGGCAGAAGGTAATATTCTAATGTTTATAAGTGGTTCTTCTCTGAGTCCTT
GGTTTCTATGTTTATGAATTCTCTTTTTGAAAGAAATTATAGTTATTATTACCAGGTCTATTCTTTTACATTGTTTC
TAATTCTATGGTGATCTTCAAAATAGAGTATCAATTTTAAATACTTGGGAATGAAATTATTCTTCCCATATCATTTC
TTTGTATGGCATACATTGTGATTTGTTGTCCCATCATTGTTTCAGTATGACCTGTTACTGCAAAAACATATTGAGAT
AAATCATCCCACATACTCTCGGCCAGGACAGACATCACACTGTTGCAGCAACACTTCAGATGAGCCCCATTCAACCT
TGTGTTTTTATAGAGAAGGATGCCACATGTTTATATTCATTTCTGAAGATTGGCTCATATTATTTATTGAAACATAC
TAGTTTAAAAATCTGTCCATTTATATAACACCTGGTCTATCTACATAACTTGAATTACATAAATATAAAACTAAACT
TCCCCTCTTCTCCAGTGTATAGCTTGCAAGCAAGTGCATGTGAAATAAATTAAAGCCTTGTTTGTGTTTTTTTCATC
ATGTGAGTACAAGACTTTTCAATAAAAATGAATTACTTTTGAACATATTTGTTTGGACAACAAACAAGAGAAAAGAT
CTATTTGATTGATAGTGGACAGAATTTTCATTAAGTTCAACAGCAGAAATACCACAATTGCATCATTCACCTTCGTG
TATCAAAAGAAAACAGAAAATTAGATGTGATGAACTCTACACAAATGTTCACTATGCATACTTTACCCATTAAATAC
ATTATCAAGAATCATGTCAGCATGACATTCTAATATAGCAGCTTTACAAAAACATGTAATCTAATCTAGGGATGCTG
TTGTCCTCTTTAAATCAGCTTCAAACATATTCTGGGTTGATATTTCTCATTCTTTTTTGATCCACATTGTTTATTCA
CATAATGATTATATTTAACTGAAGATAACAGCATTATCAAAGTGAAAGACAAAATAGATGTTTAATAGGAAAGTGAG
TATCGAATCATCTTTTTTCTACCAAAAACATCTATAATTATGAAGTATTTGGTTAATTATTTTCACAATAATTTAAA
AGTGTACAACTTGCCGATTTTTTTGTACTTTCTACTTTTCATGTCTCGCATATATCTCTTTAATATCTAAGTATTTG
AGTCAGAAAAGAGCCAGTACCGAATAATGGGAATCTCACTGAAATGTGATAACAATCTGGGGCCTGGTCCTGGGACC
TTTATCTGCAGGACAACTTGGACAAATATTTAGACCCCCAATTCCTCGTCTTTACCCTAGGAATAATAACACATTTT
TCTGACCTCATACTTCACGTGGATCTCAAATGGAACAATCATCTGATAGCACTTTATGAAGTATATGAAAGCAATAA
ATTATCACAATAAGATAATTGCAATTATTCTTTGGCATAGTATTAGTGATGTCTTTATCTGTCTGACAAAATCAACA
TTTCTGTATGGTAACTGCCTTTCCTTGTTTTAACAGAAGATCATGCCAGAAAAGATGAGTAGGTAGATACTTAACTT
GTTGTTCCTGAATCTGGAATGTATTGCAGATGTCCCAGACTGATCTTTGTTCTTTTTTTTCCTTACAAATTTCTTTT
CACATTGACAGTGTGATATTTCTTTAAATGTGCAATACATAGCTAACCTTATTTGTTTGTGTTTACTAATTAAAATA
TCTAAACTGCTTAAAGGAGAAAATTCAGTTTTAAGTTTTATTGATTTATACCCTTCTTCAATCCACATAGGATTAGG
GTAGTATGTAACAAAATTTCAAACTATAAATGAAATATTGAGTTTTGTATTAAGGCCAAGGATGAGGAAAAAAAAAG
TAAGTATATATGGAAAAAGAATGGTATTGAATGGGAGTTTTGATGGAGCATGTTGACATCATGATAATACCTATTAT
CTTTATATTCTGAATGTCAGAACAAAATTAGAGCAATTTTCCCTTATTTCCCTACAATACGTCTGTCTTAATAATTC
TAAGCTTTCCTGATTTCAGTAGTAATCTGTATTTTGCAAAAGGCAGCATGTTTATAAGATATCAAGTAAACTAAGTT
TATGGAACTTGTAACAGCATTTTTAACAACATTTCTCCCTAGATAGTTCATGGTAGACATGAATTTATTCAAAACTA
GTATGTAGAAAAATACCATTAACAAAAGCTCTGAAATTATATTAGAGGAGCTGAATAATGTTACTTGAGAAAGAATA
AAATGTTATTTATGATTTTTGGTATCTTTTACCCACTATATATGGCCATATCTCTGAAAAACTTTAGTAATATGTAC
TAATGCAAATATGGTAGTAAATTATGTCTACAGGTGCTGATACCATAGTAGATAAAGTATGATAACTTTATTTTAAA
ATATCATATTTAAATAATTAATATACAGTACTGGGAAAGACTATTTTATCTATTCTCTCACTCTTGAATAAAAAAAT
CCAGAAAAAAATACCTTGTTTTGGTAAGATTATATCAATTTATTTCCCAAATGGGTAGAGGGTTATTTTTTTCTGAT
CATAAACGTATGTCTCTTCATTATAAAAATCCACTAAAAGTGATAGAAGAAAACCAAAAGAATAAATGTAAACAATG
ATGCCATTTTCCAAAAATCACCTTCGACATTTTTCTGGATATTGATACAGTCTAAATCTCTTTTCGGAAGACTCCCT
CCTGTGTAGGTTCCCCAACTACTCTGCAATCTTATTTCCTCTTGTTCTGTTCTTGTAGAAAGGAGACCCATTGTCAC
CATGTCAAATAACACAAAATGGTGCACGTATAAGATCATTGTCTCTGTCCATTATTTGCCAGAGGACCTCAAACTTT
TTCAGGTGGTGGGCAACTGGATGTCATGCTGCTCCTTGTACAACAGAACACAATTCATTATTTATATGGTTATTTCA
TTTTAAGAAAATTTAACTTTCATTAGCTGGAAAAAAAAAGAAGTGGTTTTTAAGTTGTTTAGAAATGTGAAATTCAA
TTTTCATACTGCAAAAGAGATTCAACTGCAAACACAGGCACACATGTCTGGTGTAAGAACGAGTTGTCATACAAACC
CAAATTAGCTGCCTCCACGTTGTCTTTGTTAACAAGTGTTTGTTTGCTCCTTGTTCCATCATTCAGAAATGCTCTTT
AGCAGGAATTGATGGAACACAGTCGCAGTGACCTCTTCCTGTCTTTAAAAATCGAGATGACATTTGCCCATCTGCAG
TGTTAACATAGTTCCTCAAAGACCACTGACAGTGGGGTAGGACTGTATTGCGCAAGTTCTCTCATTTCCCTAGAATA
TAATTGGTCCAGGGCCAGAGATTTTAGCTCATTTAGAGCAGCAAGGTGCTCTTTTAAAATTCCCTCACCTATTTTGG
GCTTCATTTCCCTTATACGGTTATGCCTTTTCCAGTCTGATGAACATTCTCCTTGACAGAGCAGACAAGCAAAAGGA
GCTGCACACTGCTGCTTTCTGTGTCGTCTCTATCCCTAACCTTCTCCCTTCTGCCCCAATCAGTGAACCTTCGTCTT
TCTGGTTCTTCTTCCTCCAAATGGAAGTAAAAAGGCCCTGAATGTTGTCTTTACCATTATCACGAGCCTCAATTCAT
TCCAAGCTCAGCTTTTCCTCACTGTTTATACAGTTCTATATTGTTCTTCTAATATTTGCCCTCAGTTCTCTGTCCCT
CGTTTCTTCCCATGTTCATACTCTATTAGAATCTGAGCACCTTTGAGGTTGTCCATACAGTGGCACACATCTTTGTT
TTATACTCACTGGGATGATTTGCCATTATATTGTCAAAATTTTATTCTAAAGAGCTTTTACAGGCTTTCTTGAGCCA
TTTTCTCTTGAAATTCAAGATCGTTGAATCTCTACGCTTTTTCCTTCTTAATCTAATAAACATACACCCCCACATAC
ACACGTGTGTTCCTGAAAGACAGATGCCACTTGACTCGTCTTATAGATTGTCTAAATTGATCATTGTGTGTGGGGAT
AAAAGGGTGAATTGTATAATATCCCTGATGGTTCACGAAGTCTGTTCCTGTATAACCTGATTAGTCTTCTGAACTCT
TTTAAATTCTGTCTGCAAATGACTGAGGTTTGGCAATCAGCCTATTTCAGTTAGTTGTTTTCTTGCATAAGAAGGGT
CCATATGTACTGTGTGAAGTAAGAGAGAGAAAGTACTTAGATTTGCTGGATGCCCTGATTGTTAGCATGGCTAAGGT
ATTGTGTAAGTAAGGAGAGCAGTTAAAAATGATATTGTTTTTATTTCTTAATTGAGGTAAAATTTTATATAAGATGA
AACAGACTTATTTGGGAGAGGAGGAAGAGTTTGTTCTTACATAACATTTCAACCTGTCATATTTAGTTGAGAACTTC
AATCTGTCAAGATACTTTGTATAATATTCAGATTCTGCCATCTAATATATTTTCCACGCTTTCTTACTGGGTGTGAC
AGTAACTTATACTGTGGCAGGTGTATAAGTTAGTAAAGATATTAAATGCTCAATCTGTTAACTTTTGTGAAGTGGTC
CCACTGATAAAGTGACACCTCAATAAAATAAAAATTTCCATTACCTCAGAAAGCTTTTTCATGCTACCTTCCAGTCA
ATTCCCAGCCCCAATAGGCACCTATTCTTCTGATTTATATCACCATAGATTAGTTTTGTCTTTTTAAAAATTTGTAT
AAATGAAATCATACAAAATGTACTATTTTGATCAGCATACTACTTTTGAGATTCATCCATGTAAGTGTATCAGCTGT
TCATTCCTTTATTGATGATTAATATTCTATTGTATAGATATACCACAATTTATTTATCTATTCTCCTTTTGATGGAC
ATTCAGGTGGTTTTCAGTTTTTGGCTGTTATGAATAAGATGCTGTGGACATTTGTGTACAAGCCATTTGTGAGCATA
TGTTTTCATTTAGTTTGAGTAACTCTGTAGAAGTGGAATGGCTGGGTGAAATGTTTAAATTTATGAGATATTGTCAA
ACAGCACCTAAACAGTTTTCTAAAGTGGTTGTGCCATTTTGCAATGCCACCAGTGATGATGGAGAGTTCCAGTTACT
CTACATCTTTGTCAATATTTGGTCTTGTCAGTCATTTTAATTTTTGCTATCTTACAGAATATGTAGGTATATTGTTG
TGGTTTTAACTTATATTCCTCTGATTACTAGCACTATTAAGCATCTTTTCATGGATTTATTGGACATTCATATAGAT
TATGTGTGTTGAAGATTATTACCTTTATGATTATTGGGTGAAAATAGTATCATTTTGAGGTCATTCATATAACTTGA
AGACTGGGAATGACAGACATTTTCCTGTTTTGTTTCTTTTCTTTTTACTTTATCTGAAGAGTCTACTAGAATGCAGT
GTTGCTGCCTGAGCAGCAGGGCATTAGCTTTGTAAAAGCTCTGTTCCTTGGCAACCCCACCACTAATATGAAGTGCA
GAACATTTGAATTGTCTTTGACCAGCTTCAGCATCAGCACTATTTTTTTTTTTTGCTAGACCCCTAGTAGGTATTTA
AAAGTACAGAAATAGAATTTAATCATGCTTTTTACCAAATGTGCTATGCTCTTAGAGATTCTTTCAACGTGCATAAA
AATTCTGCAGTTTCACCACATACCAGTAAAAGAAACTCAGTCACTCATTTAGCCATTTAGTAAAAAGAACAAATTAA
CTGATGAGCATAGTGGAGACCTCAAAGGTAAAGAAGACAATGTCCCTGAAATAAAGACAATCATAAATTTTCAATCA
AAATAATGAAATTTAGGCTGGGCATGGTGGCTCATGCCTATGATCCTAGCACTTTGGAAGGCTAAGGTGGGAGGATT
GTTTGAGGCCAGGAGTTCAAGACCAGCCTCAGCAAAAAAGTGAGACCCTGTCTCCACAAAAAAATTTTAAAAATTAT
CTGGGTGTGGTGGTATGCACCGGTGGTCTCAGCTACTCAAGAGGCTGAGGTGGAGGATCACCAGAGCTCAGGGGTTG
GAGACTACAGTGAGCTATGATTGTACCACTGCACTCAAACTTGCATGACAGAATGAGTCCTTGTCTCTAATAATAAC
AAAATTTAATTTTTATAGACTGTGAAAAACCATTATGTAGATACAGTTCAAGTACAGTATGATTTTATAGGATAGAT
AACTTTTGCTTGAAAATGTATTCCCAATTTATAGGATAGATAACTTTTGCTTGAAAATGTATTCACAATAGAGTTAG
TATTTGGGGCACACCTTTATCCATTTAACAAACATGTTTTGAGCACTGCCAGGTAGCAACACGTTACTAGGCACTAG
AGTGAGAAAAGATTACAGTTCCTGCTCTCATGGATCTCATGGTCTAGTCAACTGGAATGAAAGGATTACATAAGTAG
AGGTAAAGACACACATGATGGAGGATGGAGAATAGTCAAAGGTCTGGAGAATGACCAGGACGTCACTGTGAGTTGTC
TAATTGCACTGAAGCATGGATGAAGAATTGGAAAGTCATTGTAAGAAGCCTAAAAAGGTATCTCTCAGGGATGCTAT
GAGGTTCTGAATGTTATGTACGCTATTTGGGCTTCAACAGGCAGGCACTGAGTATTCAGTATAAATTTTTGAGCAGG
GAATCCACCAGAAGAACTATGCATCTGGAGGATTAATCTGGAAAGATTGTGTAGAATGTTATGCAGTGAAAGAGTCT
GAGATGAAACAGTTAGGAGGGTGTATTAATAACATAGGTGAAGTGTAATGAATAACCAGGCTGGAGGAAAAGCAATA
ACGATGGAATCAACCGGGCAAGAAGTATAACAATTAGGATCAGTAAAATAGAATTTGGATTGGAGGAATGAAAAAAA
AAGGGACAAAACAAAGTTGAACTGCTGGTATCCATACTGGAAAATACAGATGTCATTCAAATAAATAATGTAATGAA
TATAAGAAACCAGTTTTAGGAGTGAAGTGGATGTTGGCTTGAAAATATTTCCTTTGAGGTTTCAGTCAAATGAAAAG
GTCCTGAAATGCTACGTGGTAGCCTAAGAAGGAAGCGTTCCTAGAGAGAAAAAAATTAGAAAAGATTTACATTTGAT
AATTTAATCTTTTCCTTCATACAAGCTAAATTGATAAGAAAGTAAAACCTATAGTTTTCACCACTCTTTTACAAATA
TCCCTAACCTTTTAGATATTCACATGAATAATTGAGAAAAATCTAACAGATGACTTGCTTATGTCATTTGTCTGCTT
TATCCTTAGGTTCCTCTGGCTTATATATTGTTCAATAAAATACAGATCATTGATATTGTACAATGTACTGATAATGG
GGAGTGAATCCATGCTTGTGCATTCTTTTTTTTTTTTTTTTTTGATTTGCAGAGGGCGTGCCCAGTCAACAAGAGAG
GCACAATTGTTTTTATCATCACCTCTTCTCATCTAATTCCATGAAGGAGAGTAGTATTACCATACAACAGATAATGA
GTTGGAAAACAAGAAACCTAACCTCAGAACTTAAGGCTTGGGGAAAAATAAAAGAGTAATTTGTGTTTAATGCCTGT
ATAACTTGGCAAGAGGGACATATAAGGCTTAGTGATGCCCAACATGTGCTTAGATGTGGATTGTTAGTTGATGTCTT
GGGGGTTCTGTAATCTAAGCTAAATGCTCAAAATCAATTAATTGATGTTAGACACAGAGATCTGCTTTGATCCCTCT
TTATCGTATTTCTAGGCCTTCCCATTCTCAAGAGCCTGAGAAACGACAGCTTTCCTTAATAACTTGTTATTTGTGGT
AGGAGATGAAACTTTGATAAAAACACAATTATTTTTAAATGTCTCTTTTTCACTCTAGGCTGTTGTATGTATTTCAA
AAAGTTACTTTTGACCCTTTCCAGAATGAGAAAGCAATCAAGAAGATTATAATATCTTGCTTAGTTTTCTGCTCAAT
TTATCAACAAATATTTCTTAAGCAATTATTAAGCTGAGCAGTGCTCAGCGCTGTACTTGGTGATATAGGAAATGGGG
AAAAGACTGTCTTTAAGGCCTTTATAATAGTAATTACCTCAACTTGTCTGTTTCTTTTCCTTACCATTTCGCCAAAT
TCATTGATCTATCTTGTTCTCAAAGCAATCGCCATAGTTATATTGTAACACAGCATTTTCTAGGGTGTCCCCATTAA
GTTGAGAGTGTTGACAAGAAAATACAAGCTTATTTATCATTGTAAAACTTGAGACACCTAGTAGTTACCCTAAATTA
AATATTTGTTGGAGTCAGTCACACTAAAGAGAACACTTACTGCATTGAACAATTTACCTACATTAGACAGCATTTAA
AGACTATGCCACAGCAAAGGCCCATGGAATTCTTGTGAACACAGAATAGAAGTGTATTAAGGAACAAGCTTAATTCT
GTTCTCTTAAAGCACAACACTTTCTCAAAACATATTTTGAAATCACCTTTGACCATTTTTTTTAACTAATAGGTGGG
TGGGAGTTAGGGTAGGAAAACACAAGCAGCTTCATCAAAACGATATTCTATTTTCTTCAAATTTGTGGGGAATCATA
CGGCCTCTCAATTTTCTACATTATGCTAATTATGATATTAATCTCTCTGCCAGCAAATGAAAATAATACATATTAGA
TGTAGCAAATGTCAATAATGACAAAATTAGTCATCATGCAGATACTCAGGGATTCCCAAAATATGTTTGGATTATGA
TTGCTAGCTTTGAGTTTGCCCAGAATCGTTTCAATAAAAATAAGGGACTCAAACACATTTGGAGCAAAACTCACATC
ATAAATTTTAGACATAGCTCTGCCAATAATGCTCTCAGTTATATTTTCAGTCCTAATATTTCCTCTGAGTTCCAGAC
CAGTATCTTCAACTGTCTGATTGATACTCTCTCCTTCATTTCTGTCTCCAATGCATTAAGTCCTGTGTATTTACTTT
CCAAATGCCACTTGGTTCCATGCACTTCTCTCCATTTCTGCCACTGACTCCTCCTCAATCCAAGCGACCATCTTTCC
TCACTTTAACTACCATGATATCTCCTGCTTGGTCTCCTTACTTCTATTCCCGGGCTCCTCCAATCCATTCATCCTCC
AGCAGAGAATGATGACTAGCACCTTCCACAGTGTCTGGCTAATAGGAGGTATCCAATCAATAATTGACTTACAGAGT
GAAAATATAGGCATGGCAAATACCAGTAGAGAACTACAGGGTTTTAGAACCAATGACATTAGATACTTCCATCAAAT
ATTTACAGTGTATAATCAAGTTGACTTGCACATTGTCTTATTTTTGAAAAACAATTTTGTTGGCTTTTTCTATATGC
ACACATACATATTGTATCACCCTCTACCCGCCAAATGGCTTTTGAAGAAGTATTTATGTGGCTCCAAATTGATAATA
CCTCTAGAGAGAAGAGAAATTAGAAATTTTAAAATGACCTATGCTTCCTTTCGAATATCACGTCCTGAGACAGTGTT
TTTTGAGTTACGTGCAATATGTTCCACGATGAAACATTTAATGTGTTCAGAGGCATGCTAGTAATCATGTAGAAAGA
ATTTTATGCCTGAAGTCACATGTTCTATAACCAGGATCACTTAATAAGAAAACAAGTACAGCTGTGGACAAGATGCC
TTTTTATCAGGGAAAGGCCAATTTGTTTTCTTTGCAAATCTAAGTAAATGGAGAGAAAAACACAGCCCTTAAATGTT
TTCTATTTGTCCTGAAGTTCTCATGAATGAGTTAGAAGGCGAGAAGGATTAAATAAATCCTTGAACGTAGAGAGAGC
TAACATTTATTTTAGCAAACTAAAACCTATTCGCTTTGCAAAGTTCTGTTCTGTACTTTGTAACAACAGTTTTCTTT
AAAACAAGAGCCACCAATTCAAATGCCTTTACAGAATGATTGAATGCTTTCATGCCCCACCTAAAGGCATTCAAATC
ATTAATCAAACAAAGTTCTAACGCCAAAACATGTCTGGGACCAGATTTAAAATGTAGCCCTCAGTTTCAGAGGGCAA
AAACTTAACATATTTATATTTTCCTCACTTTAGGTAACACTGTATTGAATCTCTGCTTGAAATTGAGGAGCACGTGA
TTTTTTCTTTTTGGCCCAGGGCAGCATTTCTTGGAAGAGAAAGAAAAACAACCCAAGATACCCTTACAAAACATGTA
GTACTTAAAGCTCTTTATGATGAATTAATTTTGGTATACACATTAATAGCAGTGATAATAACAAATCTATATATATA
TATATAATTGATATGAATAAGATAAATACATCAAAAGGAAATTTCATTACAATTTGATATTAGGTAAATGTCCCATT
AAAATAAATTGCTACTGTACATAATTTTCCTTCAGTTCATTGGCAGGATGTTTGCTTTGGAAAATAAACAGTCTATT
TCTAGTTTTAGAAGGAATTCTCATTATTCTTTTATAGCAACCATTATCAGGAGCAGATGGGAAATTGTACCAAGAGC
ATATCTACTATTATACCTCACAGGAAAAAGAGAGTATTAAATGAAATCTAACAAGGCCTGCTCCTGACTCTAGTTCC
TGTAACAAATGAACACACACATTTGTATGGTTTCAGCATTTGTATTAGTAAGGTACAATAAATGTTTACTGAAATTG
AAAAAAAAAAAGATAACAGGAGAAAGAAGAGGCTAAAAAGGTGCATTTTATTTCTGATCGTTCATTGTAAAGACTGC
TCCTTTTTAAAATAATCAAATTTTATTTTATATACAGAGGGTACATGTACAGGCTTGTCACAGGGGAATAGCGCATG
ATGCTGAGGTTTGGGGTACAGATCTCATCACCCAAACAGTGAGCATAGTACCTACCTGATGAGTAGTTTTTCAACCA
ATGCGCACCCTCCCTCCTTCCCACATCTACTAGTCCGCGGTATCTGTTGTTCGCATATTTACGTCCATATATGCTCT
ATGTTTAGCTCCCACTTATAAGTGAGAACATATAGTGTTTGTTTTTCCTGTTCCTGCGTTAATTTGCTTATGATTAT
GGCCTCCAACTGCATCCGTGCTTCCGCAAAGGACATGATTTCATTCTTTTTATGACTATGTAGTATTTCATGGTGTA
TATGTACCACATTTTCTTTATCCAATCTACCATTGTTTCACAACTAGATGGATTCCATGTCTTTGCTATTGTGAATA
GCACAAGACAGGACCTTTTTATTTGACTGAGTTCCTTGCAAATTACTAATAAAAGATCTGGAGGTCCTTAGTTAAAA
GTTGAATCTGTAGTGCCGTTCAAATTTAGAGATGTATTTTCTGTTCAAGAGAAGAAAGCCCTCATTCGGTCATGCTT
AATATTCAGCTGTAAAGTCCAAAACATATGAGAATGACACAAATGGAAACATTTTATAAATACCTATACAAAGGAGG
GGCACTTAGTTCCCCTAGGCCTCTTAAAAGTCCTCTAGAAAGAGGGTACTTTTATGCTAACTATTAAAGATGAGTAA
CGAATTTGTCCTATACAACTTAACAGTATCGTCAAGGAAGTAGAAAGTTACTCAGTTTTACTGGGCATTGGAGCTAA
GCTTGAAAGTGAGGAGGAGAAGCGGCAGGAGACGGAGCCGAGAAGGCAGTGGGGAGAAGAGGAGGATGGTCCTTTCC
ATGCTCCCTGTTGTACTAACATGTTTGGATATTATCTTATACTTCATATATGGACTGGATTCTTGTCCTTCTCATTC
TGAGCTCTCCTTGACCTTGATTCTTACCTCCTATAACTTTCATTCTTTCTTTACTCAAAAAAAGGCCATTTATTTCA
GCCATTTTTCACTGTTTTCTTATCCTTCCTAGTTGCTTTTCTATACTATTTTTCCACTCTTTTTTTTTTCTATACTA
TTTTGCCCTTCTCTCCATTTTCCTAACTGCTAGATTTCCCCAATTTTAGCCATCTTTCAATTGTTCTGACTATCCTC
AGGTGCTCCCACAAGGTTATCAGACCTTCCACCAAGACGGAATCCCTCAGTCTATGGACAGGCTAAGTTGAATGGGT
CCTGGTGCTGTGCTTAGCATATGCCTTGAGTATTTGTGCATTTATTTTGCTTCTTTACAAAAATCCATCATCCGATA
GAAGTTGAAAGAAACTTGCTGAAGCACATTAAAATCTCTGAAAACAGTATTGGCTATATTTTCTAATAATTAGCATG
ACTGGTTAACTTGCTTTATTTATCATTGAAAAAAGTATCAGAAACTGTATATCAAACTCCTGAATTCTTGGCACTGA
CGAAGAGACACAATGAGAATGACCTTAGGATAAAAAAACAAGATAAAGCACCATATTTGTAGGAAATTGCACCATAA
AAGTCTGTTTCACAACTCTCCCAAATTTCATTTTATTACATCTTTTCTCTTGACCAATCAGTAAACTCGGTTAATGA
TTTACCTGTCTCAAAATAATTCATGAACAAAATTACAAGTAAATCTCAGTATTGGATTCTTGAAACATCTCCTTGTT
CAATGAAGTTTCCTTTTTCTTCCCTCTATTTCCCTGTATTTATCTTTTCTTCCAGTTGCATTTTATCTCTTCTGTTT
TTTTATCTTGCTCCCTAGTTTGTGATTTTTTGCCAATTTTTTATTTCCTACATAATTCATCCAATCTGTCATTGTAC
AATTTCTTATAACTGCTTCTTAGCTTATTCCTTTTCTTCATTTGTCACATTCTATTTTTCATCTATTGTGTTTTCAT
GCAGTTTTGGAAAGTTTTACAAATAGACTTTTAAAAAAATGTACGTAATGTTTTCATAGAAAAGGTAGTGGTTTCTT
TTTCTTATATCCTTCCCTGTATAAAAATAAAAATGTAGCAGTTCTTTCTTTGCCTATGTTTCCTCTTTCCTTCCCCC
AATTTGACCAGACTTGAAGGACTTAGATATGTAACAGTGTTATTTTCTATAATTTAGGAACAGCTTTTGACTTAAAA
AGCAGAAGAGAAGTTGAAAATAATATAGTAATTCTACATGTCCTTCCTGCTTCCCAACTCTCTGCACATGTTTGTAA
CCTCCCCTTTCTTTTTTAGTGTATCTCTTTCATATACCTTTGTCCCCAGAAATTCTGATTCAGTAGACTTAGAATGG
AATTCTGGGCTTTTATATTTTGAAAAGCTCCCCACGGGAGTTAGATATGCACTTCTTATTAAGAATGAATGCTTAAT
ATTGGAATCAAAACACAATAAGCTTTCTAACTATGATGAATAATCCAACAGATTTAATTATGATTTTCTTTTTGTCC
AGAACCAAGACTAGATGTTAATTGCCAGAGAAATAGATAAGAATGCCTATGACAGCAGTACATTAATATGATATCAA
AGCTTGGAAATTTTATTGGTAATGAATAATTCAGTACTTAAAATATTTAGAAGCTATAGAATTAAAATTAATTAATG
TTGTTCACTGTGTGAATAAAGTTGATTGAGATTTTACATTTAATTTTGTAAACCCAGTGTTATCTTTTCCAGCTCAG
AAAACACCACATACAAGCTACTACTTTCTGTTTTGATCCCTTATTTTTCTTTCTTATGCTTTATCACTGAAAACTCT
CCTTGAGCAGGCCATGCACTGTAAATATTTCTCCTGGTTGCAAAACCTTCTCATACAAATGCAGTAGACTGTGTAAT
GAGCTCTTCTTTCACAAAATTAAAAAAACCTGAAAGCCCTGATTTGCGATTCTATACAAATGAGATTTAGATCTAAC
AATTTTAAATTATTGCTTCACTCTTAGCTGTTCAATTCTATCTCTTATTTGGGAAACCGAAATAATAAAACCATTGC
TGATTCCACAATTAGGTTGTAAAAGTCACCGTAGCCATCAGCCATGAAGCAAAAGTGCCAAGATCAAAACTACAAAG
CAAAGAGGCTGAGATAAAAATGCTGCAGCATTAGTTTATAGCATTATAAGCAGCAATAAGAATTCCTTGATTGCTTA
ACAAAGACTCAAAAGGCATTTACTCCATTACCTTACAACTCAAAGAGGTATTCCTGGACCAGCAGTATTGGCATTTT
TTTGAAGTTTGTAGGAAATGCAGAATTTTGGTGCCTCCACGGACCTAATGCAGCAGAACTTGCAGTTTAGTAAGATC
TCCAGGAGATTTGTATGCGCATTAAAGTCTAGGAAGCACCGCTATGGTATACATCTGATGTGTGCCCATGCATTTTT
TAAAAGTATGAAGTAATAGTTGTAAGTATTGGACACTCTTGAAGGAACAAATAAGAGCCATGGTCTTTACTCTCTAA
ATACCTCCCTGACATCTATGTTTTAGGCAAAATTTTTTTCCCATTTCAGTAGTCACTGATGCTTGCACGATGCAGTT
TATTCCAAAACAATGGTGATTCTCATGTAATAGTTCATGTTGCCTTAATAATTTACGTTGCCTCAAGTTCTCTGCCC
AGGCCCCAATATACACCGAGGGCTGTACTCCTCCCCTAACGCCTGCTCTCATACAGTGGCATAGAGCCCAGTTTTAT
GCTCTTGGTCACATCATGGAGATTGCACACCACAGGCTTTAACTTCTGCCGTACTCTCACTGCCTCTAACCCTCCAT
ATGCCTAAGTTCTACGATTCTTTAAATTCCAAATTGACCCAGAAGTCTCCTCCGCTCATCCTTTTCACTGAGATCAT
CCCTCTTCTGGCCTACCATTTGTTGATCACCTTGCTTTTTTTTTATCCTACTGTATGTAGTATAACAAATTATCACT
TGCAACTGTGTCTTATTTTTTCAACTAGATTATGTACTGCCTAAGACCTAGAAAATTGTGCTTATTTATTTGAATCT
CTAGGAGGATCAGTAATGGGTATTAATACTAATGACTCCATGGTGATGATGAGCCTGAACTTCCTCCCTTCCTTTCT
TTCTACCTCTCTCCTTTCCTCCCTTCTTTTCTTCCTCCATTCCTTCCTCTCTTCCTCCCTCCGCTTCTTCCCCACTT
CCCTTATTCATAGATTCATGCGTTCACTCAGCAAATGCTTACTGAAACCTTCCATGCATCAGACATTGTACTAAACA
ATAGGAAACTATCATGAATAAGACACAATATCTGACCTCAAAGAATTTATGATATAAAAGTAATGGCATAAACCGTG
ATTACTTTTGCACCAACCTAATATATAGACACAGTTTGTTATGACTGGTGTCTCTATTACTAAGCAATGACTGTCAC
ATGCAACGCTGATCTGAACAGGTGGTAAAGAGTGAGATGTAAGCAATGGAGCAAAGCCAACTAGTTACAAGGAAATA
TCACATGTTTACTAGAGCACATCTCATGGGCATTCAAGAGAGTATGGCCAGGACAGCTTGTGAATAGTTCAGTAACT
GTGCATAGTTTTATATTCATTGTGAGGCACCGTGTCACCGGTTTGCTGATTTACAGAGTATTTTAATTGCTAACTGT
ATGCTACCAAAATTTCCAGTATTCGAAAATAATTTTGCTTGAATGTAGAAAAAGAAAAAAGCCAAGAAATGTATGTG
AAACGAGAGTCTAAGGGAGCTTTACCTCAGTCTCAGAAAACATGCATTCCTTCCTTCATTTAGGAAGCATGTACTGG
GGTCTACTGTCAGCTTGCTATTGTGTCAAGGAGTAGGAGAATACAAAAATATTAGAGAATATGAATCACATCTATTA
GGAGAGTTTTCTACATACGCACATTATTCTGTCAGTGACATAAGGATTTGAGTCATTCAGATTTAAATACGGTAGGT
ACCTCAAGTTCTCAGATATTATTTCATTTTCTAAGGTTCGTATTTAGTTAATATGTTATTTTAATGGCCTTACAAAT
TCTAGATTATCTTTTTTAAAAAGTTAAATAGAACGTAATTGCCATTTTTATTTAATGGTAAAAAGCATTTTTGTTTT
TGTGTGTACTTGGTTGTAATATTCTCCTTTTCAATTGAGCTATTTTTCTGATACTTTACTCTTAAAATTTCATTCAG
GAAAAAAGTAAACAATATTTAAGCTTGACAATCATAAAAATGCTCTGGTGACTATAGATTATTTTAAAATTTATTAC
TGTAGCTTAGGGATATCTTGATGGGATGCTCCTGAAAGCAATTAATTCTCAGTTTTTTGTGGCTTCTAATGCAAAAT
ACATTGACGCAGACAGAATTTGAAATGAATTTTCTTCTAATATAGCAATTAATTTTATTTAAATATCTCTAGAGTTT
TTTTTTAATACTGTGACTAACCTATGTTTGTTCTTTTTCACCTCTCGTATCCACGATCACTAAGAAACCCAAATACT
TTGTTCATGTTTAAATTTTACAACATTTCATAGACTATTAAACATGGAACATCCTTGTGGGGACAAGAAATCGAATT
TGCTCTTGAAAAGGTTTCCAACTAATTGATTTGTAGGACATTATAACATCCTCTAGCTGACAAGCTTACAAAAATAA
AAACTGGAGCTAACCGAGAGGGTGCTTTTTTCCCTGACACATAAAAGGTGTCTTTCTGTCTTGTATCCTTTGGATAT
GGGCATGTCAGTTTCATAGGGAAATTTTCACATGGAGCTTTTGTATTTCTTTCTTTGCCAGTACAACTGCATGTGGT
AGCACACTGTTTAATCTTTTCTCAAATAAAAAGACATGGGGCTTCATTTTTGTTTTGCCTTTTTGGTATCTTACAG

Homo sapiens dystrophin (DMD), intron 44 target sequence 1 (nucleotide positions 1127752-1127796 of NCBI Reference Sequence: NG_012232.1)

(SEQ ID NO: 278)
TTAACTTCTTAAAGATCAGGTTCTGAAGGGTGATGGAAATTACTT

Homo sapiens dystrophin (DMD), intron 44 target sequence 2 (nucleotide positions 1127752-1127796 of NCBI Reference Sequence: NG_012232.1)

(SEQ ID NO: 323)
TGACAAGCTTACAAAAATAAAAACTGGAGCTAACCGAGAGGGTGCTTTT
TTCCCTGACACATAAAAGGTGTCTTTCTGTCTTGTATCCTTTGGATATG
GGCATGTCAGTTTCATAGGGAAATTTTCACATGGAGCTTTTGTATTTCT
TTCTTTGCCAGTACAACTGCATGTGGTAGCACACTGTTTAATCTTTTCT
CAAATAAAAAGACATGGGGCTTCATTTTTGTTTTGCCTTTTTGGTATCT
TACAG

Homo sapiens dystrophin (DMD) intron 44/exon 45 junction (nucleotide positions 1376066-1376125 of NCBI Reference Sequence: NG_012232.1)

(SEQ ID NO: 279)
TTTTTGTTTTGCCTTTTTGGTATCTTACAGGAACTCCAGGATGGCATTG
GGCAGCGGCAA

Homo sapiens dystrophin (DMD), transcript variant Dp427m, exon 45 (nucleotide positions 6683-6858 of NCBI Reference Sequence: NM_004006.2; nucleotide positions 1376096-1376271 of NCBI Reference Sequence: NG_012232.1)

(SEQ ID NO: 280)
GAACTCCAGGATGGCATTGGGCAGCGGCAAACTGTTGTCAGAACATTGA
ATGCAACTGGGGAAGAAATAATTCAGCAATCCTCAAAAACAGATGCCAG
TATTCTACAGGAAAAATTGGGAAGCCTGAATCTGCGGTGGCAGGAGGTC
TGCAAACAGCTGTCAGACAGAAAAAAGAG

Homo sapiens dystrophin (DMD), exon 45 target sequence 1 (nucleotide positions 1376096-1376145 of NCBI Reference Sequence: NG_012232.1) GAACTCCAGGATGGCATTGGGCAGCGGCAAACTGTTGTCAGAACATTGAA (SEQ ID NO: 281)

In some embodiments, an oligonucleotide useful for targeting DMD (e.g., for exon skipping) targets a splicing feature in a DMD sequence (e.g., a DMD pre-mRNA). In some embodiments, a splicing feature in a DMD sequence is an exonic splicing enhancer (ESE), a branch point, a splice donor site, or a splice acceptor site in a DMD sequence. In some embodiments, an ESE is in exon 44 of a DMD sequence (e.g., a DMD pre-mRNA). In some embodiments, a branch point is in intron 43 or intron 44 of a DMD sequence (e.g., a DMD pre-mRNA). In some embodiments, a splice donor site is across the junction of exon 43 and intron 43, in intron 43, across the junction of exon 44 and intron 44, or in intron 44 of a DMD sequence (e.g., a DMD pre-mRNA). In some embodiments, a splice acceptor site is in intron 43, across the junction of intron 43 and exon 44, in intron 44, or across the junction of intron 44 and exon 45 of a DMD sequence (e.g., a DMD pre-mRNA). In some embodiments, the oligonucleotide useful for targeting DMD promotes skipping of exon 44, such as by targeting a splicing feature (e.g., an ESE, a branch point, a splice donor site, or a splice acceptor site) in a DMD sequence (e.g., a DMD pre-mRNA). Examples of ESEs, branch points, splice donor sites, and splice acceptor sites are provided in Table 9.

In some embodiments, an oligonucleotide useful for targeting DMD (e.g., for exon skipping) targets an exonic splicing enhancer (ESE) in a DMD sequence. In some embodiments, an oligonucleotide useful for targeting DMD (e.g., for exon skipping) targets an ESE in DMD exon 44 (e.g., an ESE listed in Table 9).

In some embodiments, an oligonucleotide useful for targeting DMD (e.g., for exon skipping, such as for skipping exon 44) comprises a region of complementarity to a target sequence comprising one or more full or partial ESEs of a DMD transcript (e.g., one or more full or partial ESEs listed in Table 9). In some embodiments, the oligonucleotide comprises a region of complementarity to a target sequence comprising one or more full or partial ESEs of DMD exon 44. In some embodiments, the oligonucleotide comprises a region of complementarity to a target sequence comprising one or more full or partial ESEs as set forth in SEQ ID NOs: 286-296. In some embodiments, the oligonucleotide comprises a region of complementarity to a target sequence comprising at least 4 (e.g., 4, 5, 6, 7, or 8) consecutive nucleotides of an ESE as set forth in any one of SEQ ID NOs: 286-296. In some embodiments, the oligonucleotide comprises at least 4 (e.g., 4, 5, 6, 7, or 8) consecutive nucleotides of an ESE antisense sequence as set forth in any one of SEQ ID NOs: 306-316.

In some embodiments, the oligonucleotide comprises a region of complementarity to a target sequence comprising at least 6 (e.g., 6, 7, 8, 9, 10, 11, 12, 13, 14, 15, 16, 17, 18, 19, 20, or more) nucleotides of one or more ESEs (e.g., 2, 3, 4, or more adjacent ESEs) of DMD exon 44. In some embodiments, the oligonucleotide comprises a region of complementarity to a target sequence comprising at least 6 (e.g., 6, 7, 8, 9, 10, 11, 12, 13, 14, 15, 16, 17, 18, 19, 20, or more) nucleotides of one or more ESEs (e.g., 2, 3, 4, or more adjacent ESEs) as set forth in SEQ ID NOs: 286-296. In some embodiments, the oligonucleotide comprises at least 6 (e.g., 6, 7, 8, 9, 10, 11, 12, 13, 14, 15, 16, 17, 18, 19, 20, or more) nucleotides of one or more ESE antisense sequences (e.g., antisense sequences of 2, 3, 4, or more adjacent ESEs) as set forth in SEQ ID NOs: 306-316.

In some embodiments, an oligonucleotide useful for targeting DMD (e.g., for exon skipping, such as for skipping exon 44) is 18-35 nucleotides in length, and comprises a region of complementarity to a target sequence comprising at least 4 (e.g., 4, 5, 6, 7, or 8) consecutive nucleotides of an ESE as set forth in any one of SEQ ID NOs: 286-296. In some embodiments, an oligonucleotide useful for targeting DMD (e.g., for exon skipping, such as for skipping exon 44) is 20-30 (e.g., 20, 25, 30) nucleotides in length, and comprises a region of complementarity to a target sequence comprising at least 4 (e.g., 4, 5, 6, 7, or 8) consecutive nucleotides of an ESE as set forth in any one of SEQ ID NOs: 286-296. In some embodiments, an oligonucleotide useful for targeting DMD (e.g., for exon skipping, such as for skipping exon 44) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of an ESE as set forth in any one of SEQ ID NOs: 286-296. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, or 8) consecutive nucleotides of an ESE as set forth in any one of SEQ ID NOs: 286-296.

In some embodiments, an oligonucleotide useful for targeting DMD (e.g., for exon skipping) targets a branch point in a DMD sequence. In some embodiments, an oligonucleotide useful for targeting DMD (e.g., for exon skipping) targets a branch point in DMD intron 43 or intron 44 (e.g., a branch point listed in Table 9).

In some embodiments, an oligonucleotide useful for targeting DMD (e.g., for exon skipping, such as for skipping exon 44) comprises a region of complementarity to a target sequence comprising a full or partial branch point of a DMD transcript (e.g., a full or partial branch point listed in Table 9). In some embodiments, the oligonucleotide comprises a region of complementarity to a target sequence comprising a full or partial branch point of DMD intron 43 or intron 44. In some embodiments, the oligonucleotide comprises a region of complementarity to a target sequence comprising a full or partial branch point as set forth in any one of SEQ ID NOs: 283, 284, and 298-300. In some embodiments, the oligonucleotide comprises a region of complementarity to a target sequence comprising at least 4 (e.g., 4, 5, 6, or 7) consecutive nucleotides of a branch point as set forth in any one of SEQ ID NOs: 283, 284, and 298-300. In some embodiments, the oligonucleotide comprises at least 4 (e.g., 4, 5, 6, or 7) consecutive nucleotides of a branch point antisense sequence as set forth in any one of SEQ ID NOs: 303, 304, and 318-320.

In some embodiments, an oligonucleotide useful for targeting DMD (e.g., for exon skipping, such as for skipping exon 44) is 18-35 nucleotides in length, and comprises a region of complementarity to a target sequence comprising at least 4 (e.g., 4, 5, 6, or 7) consecutive nucleotides of a branch point as set forth in any one of SEQ ID NOs: 283, 284, and 298-300. In some embodiments, an oligonucleotide useful for targeting DMD (e.g., for exon skipping, such as for skipping exon 44) is 20-30 (e.g., 20, 25, 30) nucleotides in length, and comprises a region of complementarity to a target sequence comprising at least 4 (e.g., 4, 5, 6, or 7) consecutive nucleotides of a branch point as set forth in any one of SEQ ID NOs: 283, 284, and 298-300. In some embodiments, an oligonucleotide useful for targeting DMD (e.g., for exon skipping, such as for skipping exon 44) is 20-25 (i.e., 20, 21, 22, 23, 24, or 25) nucleotides in length, and comprises a region of complementarity to a target sequence comprising at least 4 (e.g., 4, 5, 6, or 7) consecutive nucleotides of a branch point as set forth in any one of SEQ ID NOs: 283, 284, and 298-300. In some embodiments, an oligonucleotide useful for targeting DMD (e.g., for exon skipping) is 30 nucleotides in length, and comprises a region of complementarity to a target sequence comprising at least 4 (e.g., 4, 5, 6, or 7) consecutive nucleotides of a branch point as set forth in any one of SEQ ID NOs: 283, 284, and 298-300.

In some embodiments, an oligonucleotide useful for targeting DMD (e.g., for exon skipping) targets a splice donor site in a DMD sequence. In some embodiments, an oligonucleotide useful for targeting DMD (e.g., for exon skipping) targets a splice donor site across the junction of exon 43 and intron 43, in intron 43, across the junction of exon 44 and intron 44, or in intron 44 (e.g., a splice donor site listed in Table 9).

In some embodiments, an oligonucleotide useful for targeting DMD (e.g., for exon skipping, such as for skipping exon 44) comprises a region of complementarity to a target sequence comprising a full or partial splice donor site of a DMD transcript (e.g., a full or partial splice donor site listed in Table 9). In some embodiments, the oligonucleotide comprises a region of complementarity to a target sequence comprising a full or partial splice donor site across the junction of exon 43 and intron 43, in intron 43, across the junction of exon 44 and intron 44, or in intron 44 of DMD. In some embodiments, the oligonucleotide comprises a region of complementarity to a target sequence comprising a full or partial splice donor site as set forth in SEQ ID NO: 282 or 297. In some embodiments, the oligonucleotide comprises a region of complementarity to a target sequence comprising at least 4 (e.g., 4, 5, 6, or 7) consecutive nucleotides of a splice donor site as set forth in SEQ ID NO: 282 or 297. In some embodiments, the oligonucleotide comprises at least 4 (e.g., 4, 5, 6, or 7) consecutive nucleotides of a splice donor site antisense sequence as set forth in SEQ ID NO: 302 or 317.

In some embodiments, an oligonucleotide useful for targeting DMD (e.g., for exon skipping, such as for skipping exon 44) is 18-35 nucleotides in length, and comprises a region of complementarity to a target sequence comprising at least 4 (e.g., 4, 5, 6, or 7) consecutive nucleotides of a splice donor site as set forth in SEQ ID NO: 282 or 297. In some embodiments, an oligonucleotide useful for targeting DMD (e.g., for exon skipping, such as for skipping exon 44) is 20-30 (e.g., 20, 25, 30) nucleotides in length, and comprises a region of complementarity to a target sequence comprising at least 4 (e.g., 4, 5, 6, or 7) consecutive nucleotides of a splice donor site as set forth in SEQ ID NO: 282 or 297. In some embodiments, an oligonucleotide useful for targeting DMD (e.g., for exon skipping, such as for skipping exon 44) is 20-25 (i.e., 20, 21, 22, 23, 24, or 25) nucleotides in length, and comprises a region of complementarity to a target sequence comprising at least 4 (e.g., 4, 5, 6, or 7) consecutive nucleotides of a splice donor site as set forth in SEQ ID NO: 282 or 297. In some embodiments, an oligonucleotide useful for targeting DMD (e.g., for exon skipping) is 30 nucleotides in length, and comprises a region of complementarity to a target sequence comprising at least 4 (e.g., 4, 5, 6, or 7) consecutive nucleotides of a splice donor site as set forth in SEQ ID NO: 282 or 297.

In some embodiments, an oligonucleotide useful for targeting DMD (e.g., for exon skipping) targets a splice acceptor site in a DMD sequence. In some embodiments, an oligonucleotide useful for targeting DMD (e.g., for exon skipping) targets a splice acceptor site in intron 43, across the junction of intron 43 and exon 44, in intron 44, or across the junction of intron 44 and exon 45 (e.g., a splice acceptor site listed in Table 9).

In some embodiments, an oligonucleotide useful for targeting DMD (e.g., for exon skipping, such as for skipping exon 44) comprises a region of complementarity to a target sequence comprising a full or partial splice acceptor site of a DMD transcript (e.g., a full or partial splice acceptor site listed in Table 9). In some embodiments, the oligonucleotide comprises a region of complementarity to a target sequence comprising a full or partial splice acceptor site in intron 43, across the junction of intron 43 and exon 44, in intron 44, or across the junction of intron 44 and exon 45 of DMD. In some embodiments, the oligonucleotide comprises a region of complementarity to a target sequence comprising a full or partial splice acceptor site as set forth in SEQ ID NO: 285 or 301. In some embodiments, the oligonucleotide comprises a region of complementarity to a target sequence comprising at least 4 (e.g., 4, 5, 6, 7, 8, or 9) consecutive nucleotides of a splice acceptor site as set forth in SEQ ID NO: 285 or 301. In some embodiments, the oligonucleotide comprises at least 4 (e.g., 4, 5, 6, 7, 8, or 9) consecutive nucleotides of a splice acceptor site antisense sequence as set forth in SEQ ID NO: 305 or 321.

In some embodiments, an oligonucleotide useful for targeting DMD (e.g., for exon skipping, such as for skipping exon 44) is 18-35 nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 285 or 301. In some embodiments, an oligonucleotide useful for targeting DMD (e.g., for exon skipping, such as for skipping exon 44) is 20-30 (e.g., 20, 25, 30) nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 285 or 301. In some embodiments, an oligonucleotide useful for targeting DMD (e.g., for exon skipping, such as for skipping exon 44) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of a splice acceptor site as set forth in SEQ ID NO: 285 or 301. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 285 or 301.

In some embodiments, an oligonucleotide useful for targeting DMD (e.g., for exon skipping) comprises a region of complementarity to a junction of an exon and an intron of a DMD RNA (e.g., any one of the exon/intron junctions provided by SEQ ID NOs: 268, 272, 276, and 279). In some embodiments, an oligonucleotide useful for targeting DMD (e.g., for exon skipping) comprises a region of complementarity to at least 10 (e.g., 10, 11, 12, 13, 14, 15, 16, 17, 18, 19, 20, 21, 22, 23, 24, 25, or more) consecutive nucleosides of a junction of an exon and an intron of a DMD RNA (e.g., any one of the exon/intron junctions provided by SEQ ID NOs: 268, 272, 276, and 279). In some embodiments, an oligonucleotide useful for targeting DMD (e.g., for exon skipping) is complementary to any one of SEQ ID NOs: 268, 272, 276, and 279.

In some embodiments, an oligonucleotide useful for targeting DMD (e.g., for exon skipping) comprises a region of complementarity to a target sequence of a DMD RNA (e.g., a target sequence provided by any one of SEQ ID NOs: 270, 271, 274, 275, 278, 281, and 323). In some embodiments, an oligonucleotide useful for targeting DMD (e.g., for exon skipping) comprises a region of complementarity to at least 10 (e.g., 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 270, 271, 274, 275, 278, 281, and 323). In some embodiments, an oligonucleotide useful for targeting DMD (e.g., for exon skipping) is complementary to any one of SEQ ID NOs: 270, 271, 274, 275, 278, 281, and 323.

TABLE 9
Example target sequence motifs

| | | SEQ | | SEQ | Motif |
|---|---|---|---|---|---|
| | | ID | Motif | ID | Antisense |
| Location in DMD | Type | NO: | Sequence† | NO: | Sequence† |
| Across exon 43/intron | Splice Donor | 282 | GGGTAGG | 302 | CCTACCC |
| 43 junction | | | | | |
| Intron 43 | Branch Point | 283 | TATTAAT | 303 | ATTAATA |
| Intron 43 | Branch point | 284 | TCTTGAT | 304 | ATCAAGA |
| Across intron 43/exon | Splice Acceptor | 285 | ACCTGCAGG | 305 | CCTGCAGGT |
| 44 junction | | | | | |
| Exon 44 | ESE | 286 | TGACAGA | 306 | TCTGTCA |
| Exon 44 | ESE | 287 | CGGCGTT | 307 | AACGCCG |
| Exon 44 | ESE | 288 | TCAGTGG | 308 | CCACTGA |
| Exon 44 | ESE | 289 | GGCTAACA | 309 | TGTTAGCC |
| Exon 44 | ESE | 290 | ACAGAAG | 310 | CTTCTGT |
| Exon 44 | ESE | 291 | TCTCAGA | 311 | TCTGAGA |
| Exon 44 | ESE | 292 | CTCAGAA | 312 | TTCTGAG |
| Exon 44 | ESE | 293 | GACACAA | 313 | TTGTGTC |
| Exon 44 | ESE | 294 | AATTCCTG | 314 | CAGGAATT |
| Exon 44 | ESE | 295 | CTGAGAA | 315 | TTCTCAG |
| Exon 44 | ESE | 296 | GTATCTTA | 316 | TAAGATAC |
| Across exon 44/intron | Splice Donor | 297 | AGGTAAG | 317 | CTTACCT |
| 44 junction | | | | | |
| Intron 44 | Branch Point | 298 | CTGAC | 318 | GTCAG |
| Intron 44 | Branch Point | 299 | CTAAC | 319 | GTTAG |
| Intron 44 | Branch Point | 300 | CCCTGAC | 320 | GTCAGGG |
| Across intron 44/exon | Splice Acceptor | 301 | TCTTACAGG | 321 | CCTGTAAGA |
| 45 junction | | | | | |

†Each thymine base (T) in any one of the sequences provided in Table 9 may independently and optionally be replaced with a uracil base (U). Motif sequences and antisense sequences listed in Table 9 contain T's, but binding of a motif sequence in RNA and/or DNA is contemplated.

In some embodiments, any one of the oligonucleotides useful for targeting DMD (e.g., for exon skipping) is a phosphorodiamidate morpholino oligomer (PMO).

In some embodiments, the oligonucleotide may have region of complementarity to a mutant DMD allele, for example, a DMD allele with at least one mutation in any of exons 1-79 of DMD in humans that leads to a frameshift and improper RNA splicing/processing.

In some embodiments, any one of the oligonucleotides can be in salt form, e.g., as sodium, potassium, or magnesium salts.

In some embodiments, the 5′ or 3′ nucleoside (e.g., terminal nucleoside) of any one of the oligonucleotides described herein is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of any one of the oligonucleotides described herein is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR1 antibody, e.g., via the amine group.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, 20 to 25 nucleotides in length, etc.

In some embodiments, a nucleic acid sequence of an oligonucleotide for purposes of the present disclosure is “complementary” to a target nucleic acid when it is specifically hybridizable to the target nucleic acid. In some embodiments, an oligonucleotide hybridizing to a target nucleic acid (e.g., an mRNA or pre-mRNA molecule) results in modulation of activity or expression of the target (e.g., decreased mRNA translation, altered pre-mRNA splicing, exon skipping, target mRNA degradation, etc.). In some embodiments, a nucleic acid sequence of an oligonucleotide has a sufficient degree of complementarity to its target nucleic acid such that it does not hybridize non-target sequences under conditions in which avoidance of non-specific binding is desired, e.g., under physiological conditions. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid. In certain embodiments, oligonucleotides comprise one or more mismatched nucleobases relative to the target nucleic acid. In certain embodiments, activity relating to the target is reduced by such mismatch, but activity relating to a non-target is reduced by a greater amount (i.e., selectivity for the target nucleic acid is increased and off-target effects are decreased).

In some embodiments, an oligonucleotide comprises region of complementarity to a target nucleic acid that is in the range of 8 to 15, 8 to 30, 8 to 40, or 10 to 50, or 5 to 50, 15 to 20, 20 to 25, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of the any one of the oligonucleotides described herein (e.g., the oligonucleotides listed in Table 8). In some embodiments, the oligonucleotide is complementary (e.g., at least 85% at least 90%, at least 95%, or 100%) to a target sequence of the any one of the oligonucleotides provided by SEQ ID NO: 196-267. In some embodiments, such target sequence is 100% complementary to an oligonucleotide listed in Table 8. In some embodiments, such target sequence is 100% complementary to an oligonucleotide provided by SEQ ID NO: 196-267. In some embodiments, the oligonucleotide is complementary (e.g., at least 85% at least 90%, at least 95%, or 100%) to a target sequence provided herein (e.g., a target sequence listed in Table 8). In some embodiments, the oligonucleotide is complementary (e.g., at least 85% at least 90%, at least 95%, or 100%) to any one of SEQ ID NO: 160-195.

In some embodiments, an oligonucleotide useful for targeting DMD (e.g., for exon skipping) comprises a region of complementarity to a target sequence of a DMD RNA (e.g., a target sequence provided by any one of SEQ ID NOs: 160-195). In some embodiments, an oligonucleotide useful for targeting DMD (e.g., for exon skipping) comprises a region of complementarity to at least 8 (e.g., 8, 9, 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 160-195). In some embodiments, an oligonucleotide useful for targeting DMD (e.g., for exon skipping) is complementary to any one of SEQ ID NOs: 160-195.

In some embodiments, an oligonucleotide useful for targeting DMD (e.g., for exon skipping) comprises a sequence comprising at least 8 (e.g., 8, 9, 10, 11, 12, 13, 14, 15, 16, 17, 18, 19, 20, 21, 22, 23, 24, 25, or more) consecutive nucleobases of a DMD-targeting sequence provided herein (e.g., an antisense sequence listed in Table 8). In some embodiments, the oligonucleotide comprises a sequence comprising at least 8 (e.g., 8, 9, 10, 11, 12, 13, 14, 15, 16, 17, 18, 19, 20, 21, 22, 23, 24, 25, or more) consecutive nucleobases of any one of SEQ ID NOs: 196-267. In some embodiments, the oligonucleotide comprises the sequence of any one of SEQ ID NOs: 196-267.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotides listed in Table 8) may independently and optionally be uracil bases (U's), and/or any one or more of the U's in the oligonucleotides provided herein may independently and optionally be T's. In some embodiments, any one or more of the thymine bases (T's) in any one of the oligonucleotides provided by SEQ ID NOs: 232-267 or in an oligonucleotide complementary to any one of SEQ ID NOs: 160-195 may optionally be uracil bases (U's), and/or any one or more of the U's in the oligonucleotides may optionally be T's. In some embodiments, any one or more of the uracil bases (U's) in any one of the oligonucleotides provided by SEQ ID NOs: 196-231 or in an oligonucleotide complementary to any one of SEQ ID NOs: 160-195 may optionally be thymine bases (T's), and/or any one or more of the T's in the oligonucleotides may optionally be U's.

b. Oligonucleotide Modifications:

The oligonucleotides described herein may be modified, e.g., comprise a modified sugar moiety, a modified internucleoside linkage, a modified nucleotide or nucleoside and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide or nucleoside modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide or nucleoside modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides or nucleosides of the oligonucleotide are modified nucleotides/nucleosides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides or nucleosides of the oligonucleotide are modified nucleotides/nucleosides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides or nucleosides of the oligonucleotide are modified nucleotides/nucleosides. Optionally, the oligonucleotides may have every nucleotide or nucleoside except 1, 2, 3, 4, 5, 6, 7, 8, 9, or 10 nucleotides/nucleosides modified. Oligonucleotide modifications are described further herein.

c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside. The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleosides. In some embodiments, the oligonucleotide comprises phosphorothioate internucleoside linkages between all nucleosides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides comprise nucleoside units that are joined together by either substantially all Sp or substantially all Rp phosphorothioate intersugar linkages are provided. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Mixmers

In some embodiments, an oligonucleotide described herein may be a mixmer or comprise a mixmer sequence pattern. In general, mixmers are oligonucleotides that comprise both naturally and non-naturally occurring nucleosides or comprise two different types of non-naturally occurring nucleosides typically in an alternating pattern. Mixmers generally have higher binding affinity than unmodified oligonucleotides and may be used to specifically bind a target molecule, e.g., to block a binding site on the target molecule. Generally, mixmers do not recruit an RNase to the target molecule and thus do not promote cleavage of the target molecule. Such oligonucleotides that are incapable of recruiting RNase H have been described, for example, see WO2007/112754 or WO2007/112753.

In some embodiments, the mixmer comprises or consists of a repeating pattern of nucleoside analogues and naturally occurring nucleosides, or one type of nucleoside analogue and a second type of nucleoside analogue. However, a mixmer need not comprise a repeating pattern and may instead comprise any arrangement of modified nucleoside s and naturally occurring nucleoside s or any arrangement of one type of modified nucleoside and a second type of modified nucleoside. The repeating pattern, may, for instance be every second or every third nucleoside is a modified nucleoside, such as LNA, and the remaining nucleoside s are naturally occurring nucleosides, such as DNA, or are a 2′ substituted nucleoside analogue such as 2′-MOE or 2′ fluoro analogues, or any other modified nucleoside described herein. It is recognized that the repeating pattern of modified nucleoside, such as LNA units, may be combined with modified nucleoside at fixed positions—e.g. at the 5′ or 3′ termini.

In some embodiments, a mixmer does not comprise a region of more than 5, more than 4, more than 3, or more than 2 consecutive naturally occurring nucleosides, such as DNA nucleosides. In some embodiments, the mixmer comprises at least a region consisting of at least two consecutive modified nucleosides, such as at least two consecutive LNAs. In some embodiments, the mixmer comprises at least a region consisting of at least three consecutive modified nucleoside units, such as at least three consecutive LNAs.

In some embodiments, the mixmer does not comprise a region of more than 7, more than 6, more than 5, more than 4, more than 3, or more than 2 consecutive nucleoside analogues, such as LNAs. In some embodiments, LNA units may be replaced with other nucleoside analogues, such as those referred to herein.

Mixmers may be designed to comprise a mixture of affinity enhancing modified nucleosides, such as in non-limiting example LNA nucleosides and 2′-O-Me nucleosides. In some embodiments, a mixmer comprises modified internucleoside linkages (e.g., phosphorothioate internucleoside linkages or other linkages) between at least two, at least three, at least four, at least five or more nucleosides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleosides. For example, in some embodiments, a mixmer may comprise morpholino nucleosides mixed (e.g., in an alternating manner) with one or more other nucleosides (e.g., DNA, RNA nucleosides) or modified nucleosides (e.g., LNA, 2′-O-Me nucleosides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

i. Multimers

In some embodiments, molecular payloads may comprise multimers (e.g., concatemers) of 2 or more oligonucleotides connected by a linker. In this way, in some embodiments, the oligonucleotide loading of a complex can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via an oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

### C. Linkers

Complexes described herein generally comprise a linker that covalently links any one of the anti-TfR1 antibodies described herein to a molecular payload. A linker comprises at least one covalent bond. In some embodiments, a linker may be a single bond, e.g., a disulfide bond or disulfide bridge, that covalently links an anti-TfR1 antibody to a molecular payload. However, in some embodiments, a linker may covalently link any one of the anti-TfR1 antibodies described herein to a molecular payload through multiple covalent bonds. In some embodiments, a linker may be a cleavable linker. However, in some embodiments, a linker may be a non-cleavable linker. A linker is typically stable in vitro and in vivo, and may be stable in certain cellular environments. Additionally, typically a linker does not negatively impact the functional properties of either the anti-TfR1 antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A linker typically will contain two different reactive species that allow for attachment to both the anti-TfR1 antibody and a molecular payload. In some embodiments, the two different reactive species may be a nucleophile and/or an electrophile. In some embodiments, a linker contains two different electrophiles or nucleophiles that are specific for two different nucleophiles or electrophiles. In some embodiments, a linker is covalently linked to an anti-TfR1 antibody via conjugation to a lysine residue or a cysteine residue of the anti-TfR1 antibody. In some embodiments, a linker is covalently linked to a cysteine residue of an anti-TfR1 antibody via a maleimide-containing linker, wherein optionally the maleimide-containing linker comprises a maleimidocaproyl or maleimidomethyl cyclohexane-1-carboxylate group. In some embodiments, a linker is covalently linked to a cysteine residue of an anti-TfR1 antibody or thiol functionalized molecular payload via a 3-arylpropionitrile functional group. In some embodiments, a linker is covalently linked to a lysine residue of an anti-TfR1 antibody. In some embodiments, a linker is covalently linked to an anti-TfR1 antibody and/or (e.g., and) a molecular payload, independently, via an amide bond, a carbamate bond, a hydrazide, a triazole, a thioether, and/or a disulfide bond.

i. Cleavable Linkers

A cleavable linker may be a protease-sensitive linker, a pH-sensitive linker, or a glutathione-sensitive linker. These linkers are typically cleavable only intracellularly and are preferably stable in extracellular environments, e.g., extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include j-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

In some embodiments, a linker comprises a valine-citrulline sequence (e.g., as described in U.S. Pat. No. 6,214,345, incorporated herein by reference). In some embodiments, before conjugation, a linker comprises a structure of:

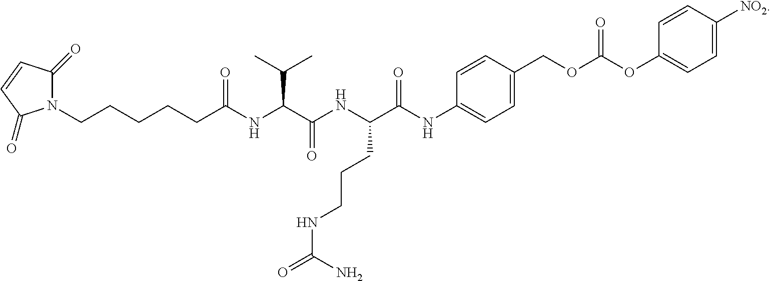 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, a linker comprises a structure of:

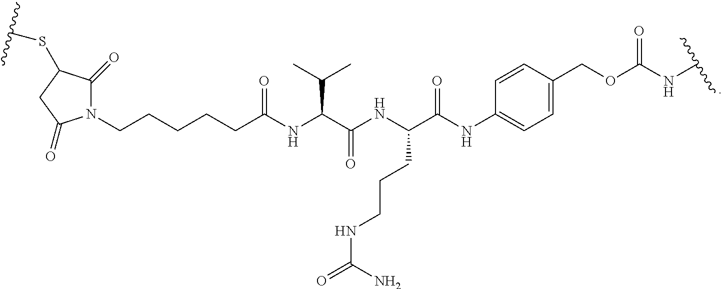 [SMILES: *NC(=O)OCc1ccc(NC(=O)[C@H](CCCNC(N)=O)NC(=O)[C@@H](NC(=O)CCCCCN2C(=O)CC(S*)C2=O)C(C)C)cc1 ; "."]

In some embodiments, before conjugation, a linker comprises a structure of:

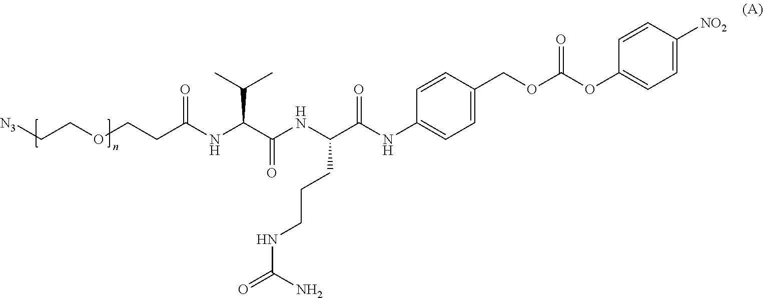

wherein n is any number from 0-10. In some embodiments, n is 3.

In some embodiments, a linker comprises a structure of:

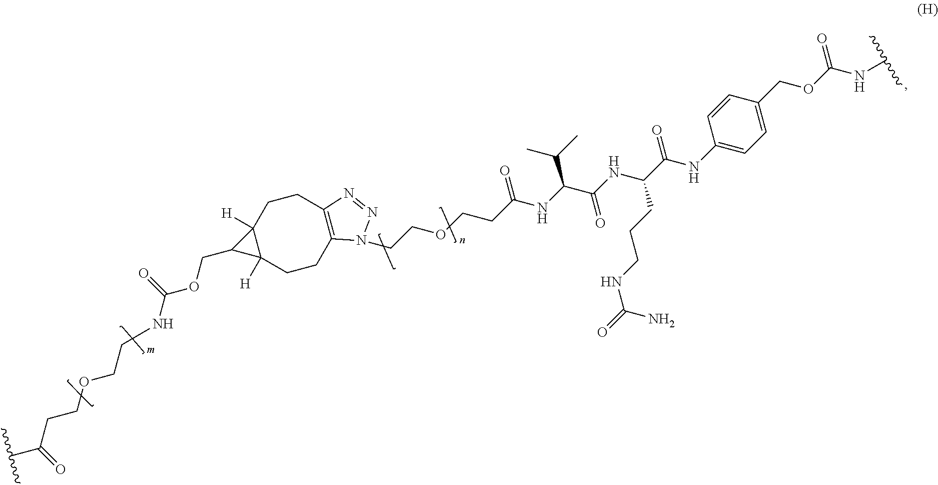

wherein n is any number from 0-10, wherein m is any number from 0-10. In some embodiments, n is 3 and/or (e.g., and) m is 4.

In some embodiments, a linker comprises a structure of:

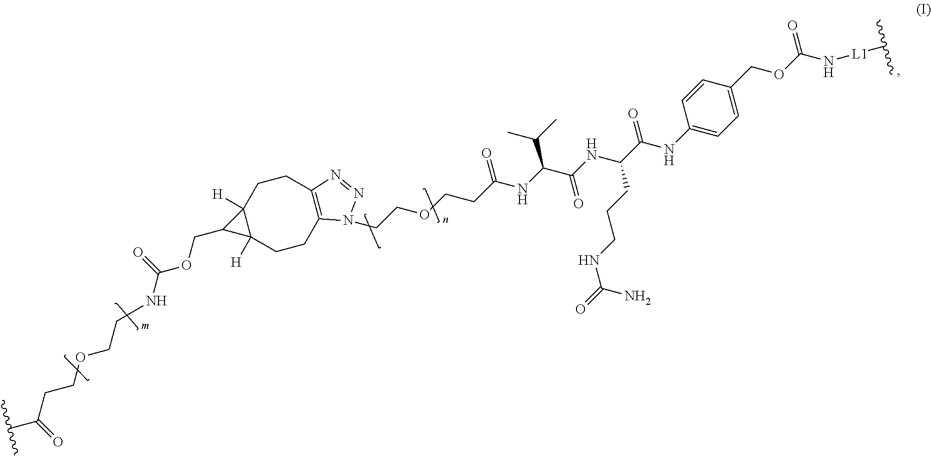

wherein n is any number from 0-10, wherein m is any number from 0-10. In some embodiments, n is 3 and/or (e.g., and) m is 4.
ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation can be utilized to covalently link an anti-TfR1 antibody comprising a LPXT sequence to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S, an optionally substituted heterocyclylene further comprising at least one heteroatom selected from N, O, and S, an imino, an optionally substituted nitrogen species, an optionally substituted oxygen species O, an optionally substituted sulfur species, or a poly(alkylene oxide), e.g. polyethylene oxide or polypropylene oxide. In some embodiments, a linker may be a non-cleavable N-gamma-maleimidobutyryl-oxysuccinimide ester (GMBS) linker.

iii. Linker Conjugation

In some embodiments, a linker is covalently linked to an anti-TfR1 antibody and/or (e.g., and) molecular payload via a phosphate, thioether, ether, carbon-carbon, carbamate, or amide bond. In some embodiments, a linker is covalently linked to an oligonucleotide through a phosphate or phosphorothioate group, e.g. a terminal phosphate of an oligonucleotide backbone. In some embodiments, a linker is covalently linked to an anti-TfR1 antibody, through a lysine or cysteine residue present on the anti-TfR1 antibody.

In some embodiments, a linker, or a portion thereof is covalently linked to an anti-TfR1 antibody and/or (e.g., and) molecular payload by a cycloaddition reaction between an azide and an alkyne to form a triazole, wherein the azide or the alkyne may be located on the anti-TfR1 antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctyne is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide or the alkyne may be located on the anti-TfR1 antibody, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is covalently linked to an anti-TfR1 antibody and/or (e.g., and) molecular payload by the Diels-Alder reaction between a dienophile and a diene/hetero-diene, wherein the dienophile or the diene/hetero-diene may be located on the anti-TfR1 antibody, molecular payload, or the linker. In some embodiments a linker is covalently linked to an anti-TfR1 antibody and/or (e.g., and) molecular payload by other pericyclic reactions such as an ene reaction. In some embodiments, a linker is covalently linked to an anti-TfR1 antibody and/or (e.g., and) molecular payload by an amide, thioamide, or sulfonamide bond reaction. In some embodiments, a linker is covalently linked to an anti-TfR1 antibody and/or (e.g., and) molecular payload by a condensation reaction to form an oxime, hydrazone, or semicarbazide group existing between the linker and the anti-TfR1 antibody and/or (e.g., and) molecular payload.

In some embodiments, a linker is covalently linked to an anti-TfR1 antibody and/or (e.g., and) molecular payload by a conjugate addition reaction between a nucleophile, e.g. an amine or a hydroxyl group, and an electrophile, e.g. a carboxylic acid, carbonate, or an aldehyde. In some embodiments, a nucleophile may exist on a linker and an electrophile may exist on an anti-TfR1 antibody or molecular payload prior to a reaction between a linker and an anti-TfR1 antibody or molecular payload. In some embodiments, an electrophile may exist on a linker and a nucleophile may exist on an anti-TfR1 antibody or molecular payload prior to a reaction between a linker and an anti-TfR1 antibody or molecular payload. In some embodiments, an electrophile may be an azide, pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, and/or a thiol group.

In some embodiments, a linker comprises a valine-citrulline sequence covalently linked to a reactive chemical moiety (e.g., an azide moiety or a BCN moiety for click chemistry). In some embodiments, a linker comprising a valine-citrulline sequence covalently linked to a reactive chemical moiety (e.g., an azide moiety for click chemistry) comprises a structure of:

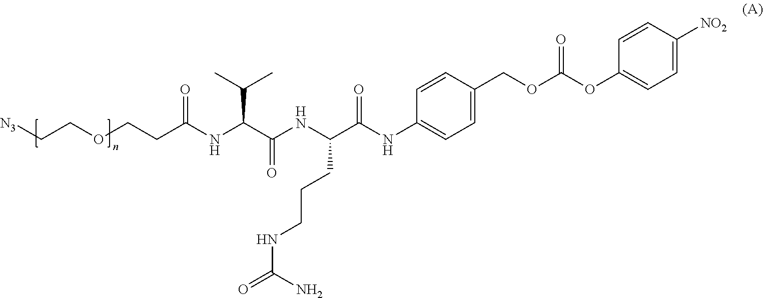

wherein n is any number from 0-10. In some embodiments, n is 3.

In some embodiments, a linker comprising the structure of Formula (A) is covalently linked (e.g., optionally via additional chemical moieties) to a molecular payload (e.g., an oligonucleotide). In some embodiments, a linker comprising the structure of Formula (A) is covalently linked to an oligonucleotide, e.g., through a nucleophilic substitution with amine-L1-oligonucleotides forming a carbamate bond, yielding a compound comprising a structure of:

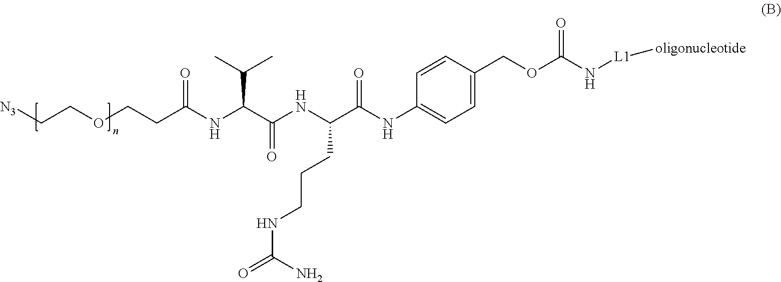

wherein n is any number from 0-10. In some embodiments, n is 3.

In some embodiments, the compound of Formula (B) is further covalently linked via a triazole to additional moieties, wherein the triazole is formed by a click reaction between the azide of Formula (A) or Formula (B) and an alkyne provided on a bicyclononyne. In some embodiments, a compound comprising a bicyclononyne comprises a structure of:

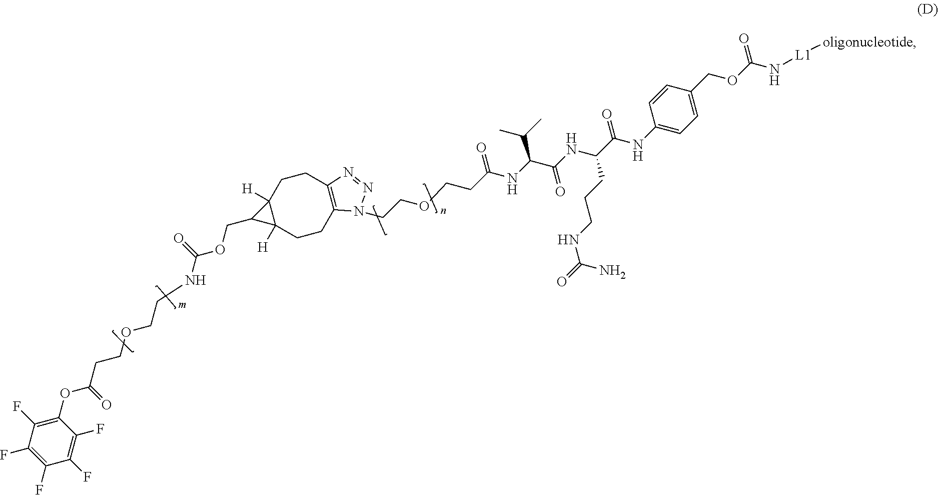

wherein m is any number from 0-10. In some embodiments, m is 4.

In some embodiments, the azide of the compound of structure (B) forms a triazole via a click reaction with the alkyne of the compound of structure (C), forming a compound comprising a structure of:

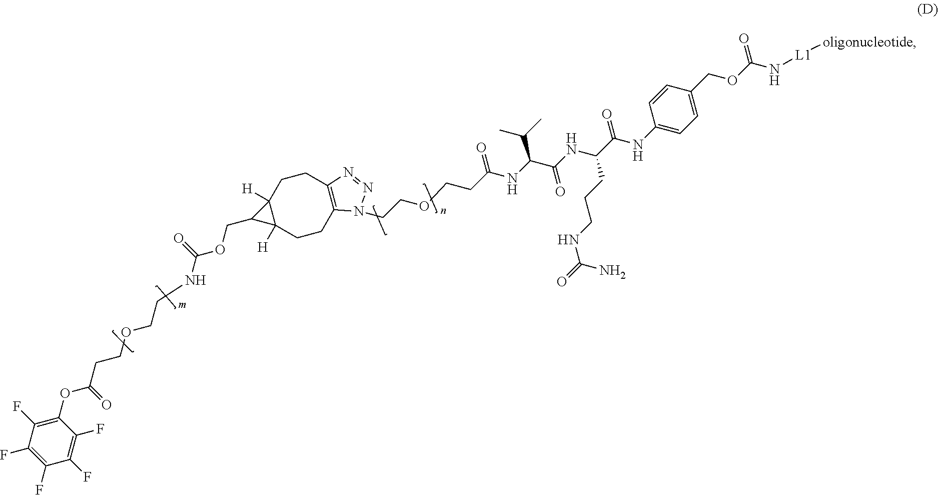

wherein n is any number from 0-10, and wherein m is any number from 0-10. In some embodiments, n is 3 and m is 4.

In some embodiments, the compound of structure (D) is further covalently linked to a lysine of the anti-TfR1 antibody, forming a complex comprising a structure of:

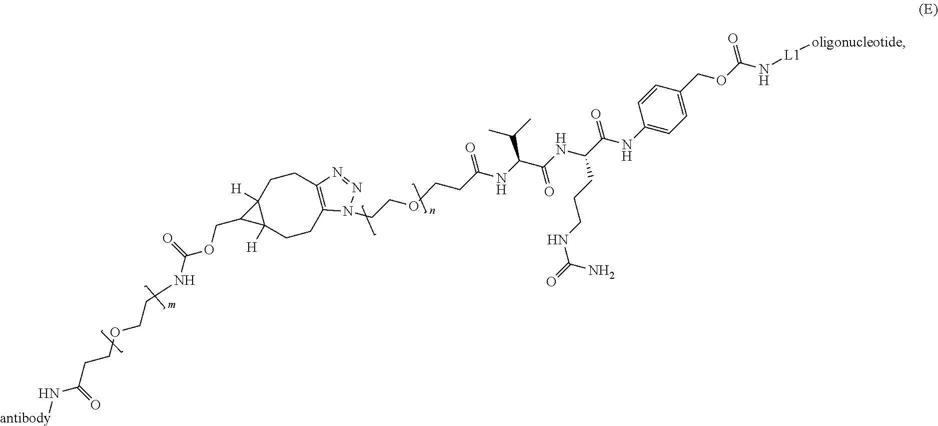

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the compound of Formula (C) is further covalently linked to a lysine of the anti-TfR1 antibody, forming a compound comprising a structure of:

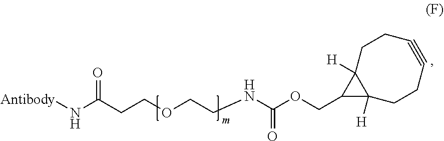

wherein m is 0-15 (e.g., 4). It should be understood that the amide shown adjacent the anti-TfR1 antibody in Formula (F) results from a reaction with an amine of the anti-TfR1 antibody, such as a lysine epsilon amine.

In some embodiments, the azide of the compound of structure (B) forms a triazole via a click reaction with the alkyne of the compound of structure (F), forming a complex comprising a structure of:

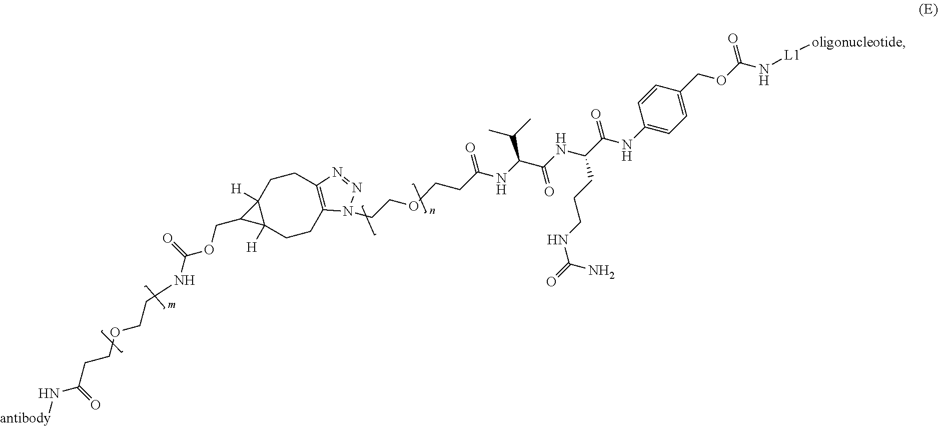

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the azide of the compound of structure (A) forms a triazole via a click reaction with the alkyne of the compound of structure (F), forming a compound comprising a structure of:

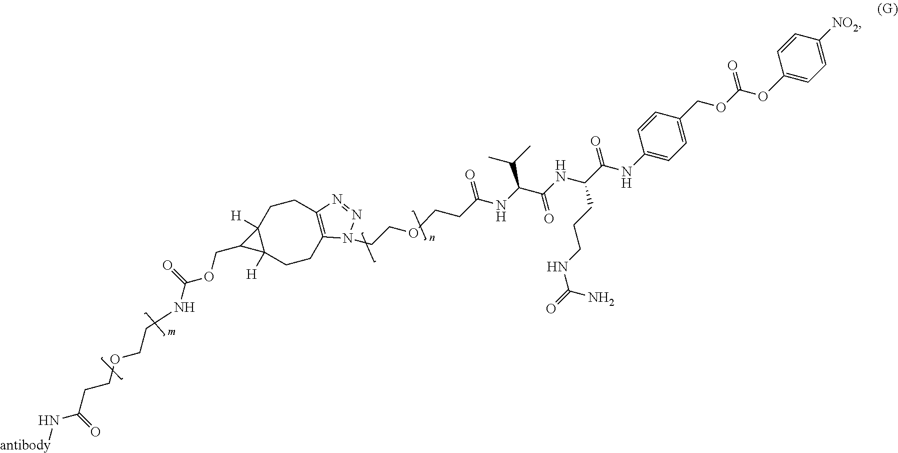

wherein n is any number from 0-10, wherein m is any number from 0-10. In some embodiments, n is 3 and/or (e.g., and) m is 4. In some embodiments, an oligonucleotide is covalently linked to a compound comprising a structure of formula (G), thereby forming a complex comprising a structure of formula (E). It should be understood that the amide shown adjacent the anti-TfR1 antibody in Formula (G) results from a reaction with an amine of the anti-TfR1 antibody, such as a lysine epsilon amine.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

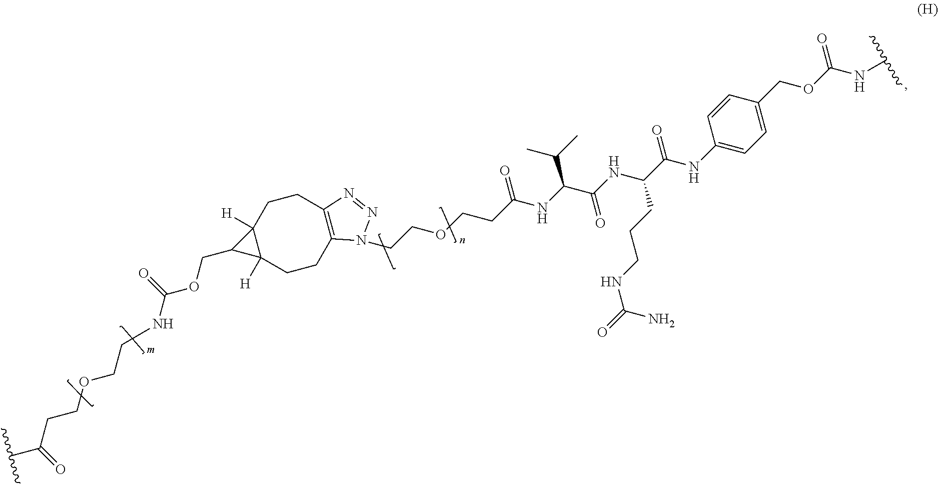

wherein n is any number from 0-10, wherein m is any number from 0-10. In some embodiments, n is 3 and/or (e.g., and) m is 4.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

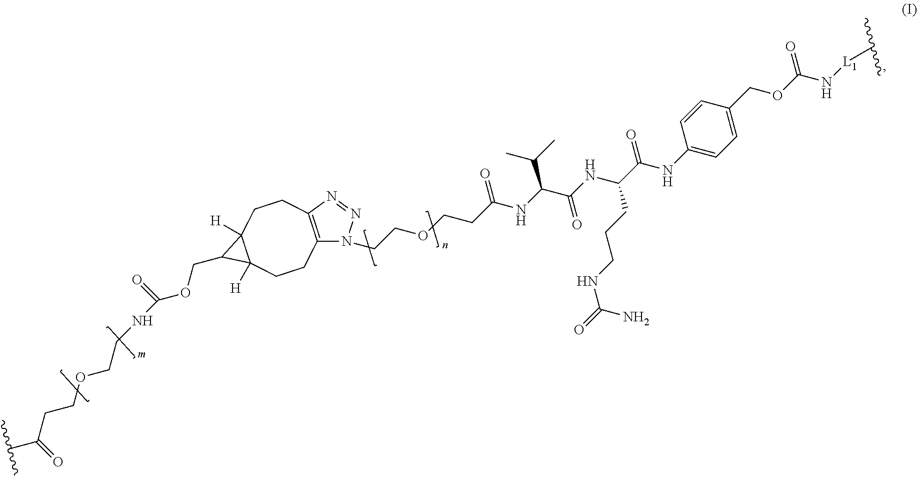

wherein n is any number from 0-10, wherein m is any number from 0-10. In some embodiments, n is 3 and/or (e.g., and) m is 4.

In some embodiments, in formulae (B), (D), (E), and (I), Li is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl. In some embodiments, L1 is

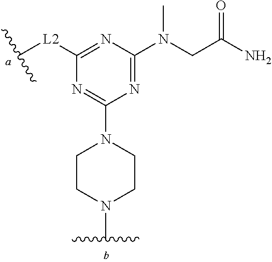

wherein L2 is

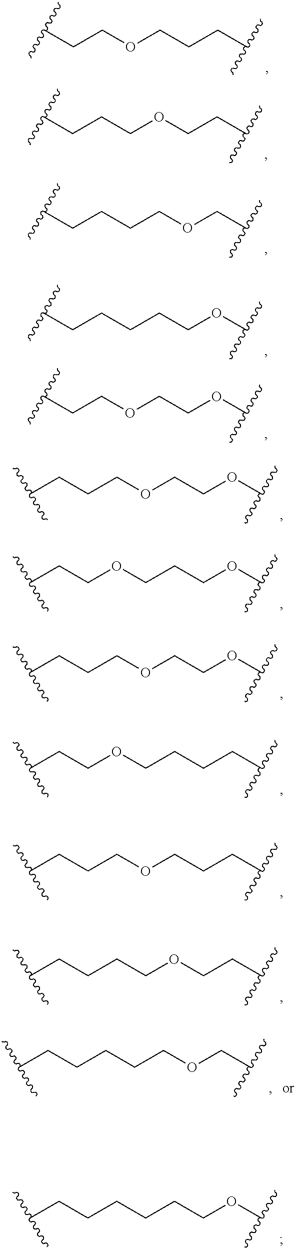

wherein a labels the site directly linked to the carbamate moiety of formulae (B), (D), (E), and (I); and b labels the site covalently linked (directly or via additional chemical moieties) to the oligonucleotide.

In some embodiments, L1 is:

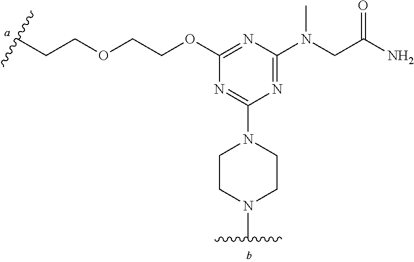 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

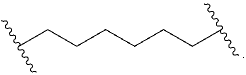 [SMILES: *CCCCCC*]

In some embodiments, L1 is linked to a 5′ phosphate of the oligonucleotide. In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

In some embodiments, any one of the complexes described herein has a structure of:

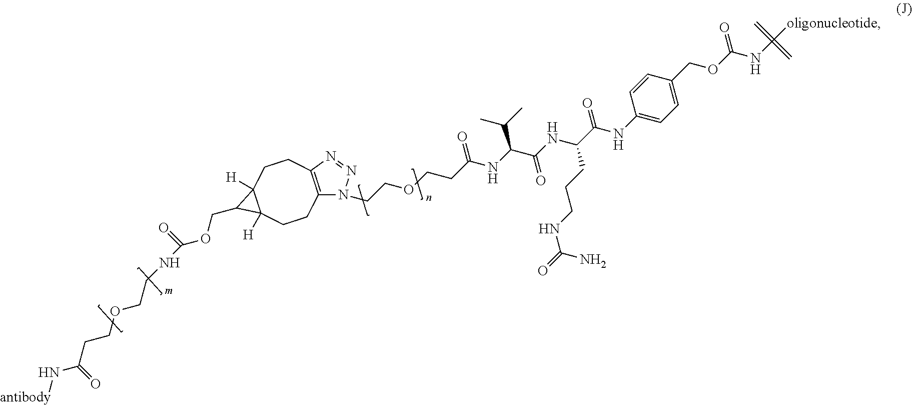

wherein n is 0-15 (e.g., 3) and m is 0-15 (e.g., 4). It should be understood that the amide shown adjacent the anti-TfR1 antibody in Formula (J) results from a reaction with an amine of the anti-TfR1 antibody, such as a lysine epsilon amine.

In some embodiments, any one of the complexes described herein has a structure of:

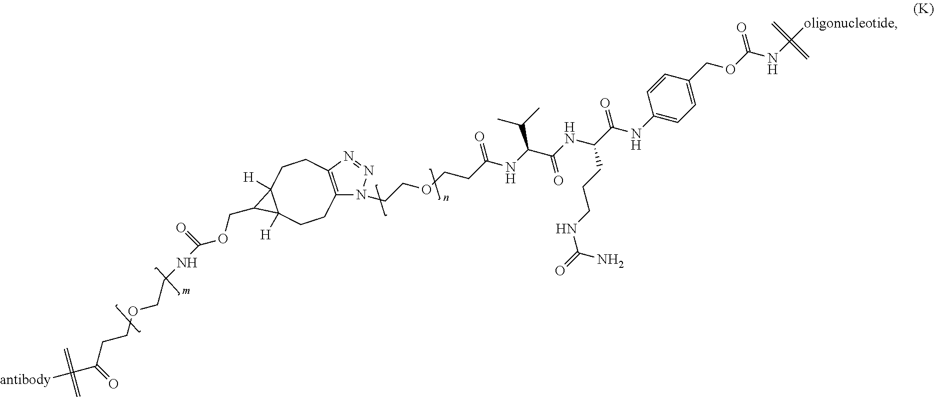

wherein n is 0-15 (e.g., 3) and m is 0-15 (e.g., 4).

In some embodiments, the oligonucleotide is modified to comprise an amine group at the 5′ end, the 3′ end, or internally (e.g., as an amine functionalized nucleobase), prior to linking to a compound, e.g., a compound of formula (A) or formula (G).

Although linker conjugation is described in the context of anti-TfR1 antibodies and oligonucleotide molecular payloads, it should be understood that use of such linker conjugation on other muscle-targeting agents, such as other muscle-targeting antibodies, and/or on other molecular payloads is contemplated.

### D. Examples of Antibody-Molecular Payload Complexes

Further provided herein are non-limiting examples of complexes comprising any one the anti-TfR1 antibodies described herein covalently linked to any of the molecular payloads (e.g., an oligonucleotide) described herein. In some embodiments, the anti-TfR1 antibody (e.g., any one of the anti-TfR1 antibodies provided in Tables 2-7) is covalently linked to a molecular payload (e.g., an oligonucleotide such as the oligonucleotides provided in Table 8) via a linker. Any of the linkers described herein may be used. In some embodiments, if the molecular payload is an oligonucleotide, the linker is linked to the 5′ end of the oligonucleotide, the 3′ end of the oligonucleotide, or to an internal site of the oligonucleotide. In some embodiments, the linker is linked to the anti-TfR1 antibody via a thiol-reactive linkage (e.g., via a cysteine in the anti-TfR1 antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

An example of a structure of a complex comprising an anti-TfR1 antibody covalently linked to a molecular payload via a linker is provided below:

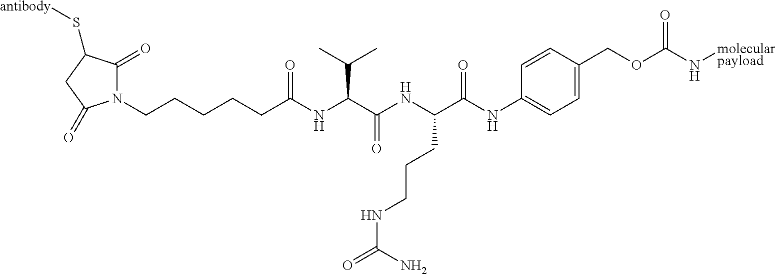

wherein the linker is linked to the antibody via a thiol-reactive linkage (e.g., via a cysteine in the antibody). In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

Another example of a structure of a complex comprising an anti-TfR1 antibody covalently linked to a molecular payload via a linker is provided below:

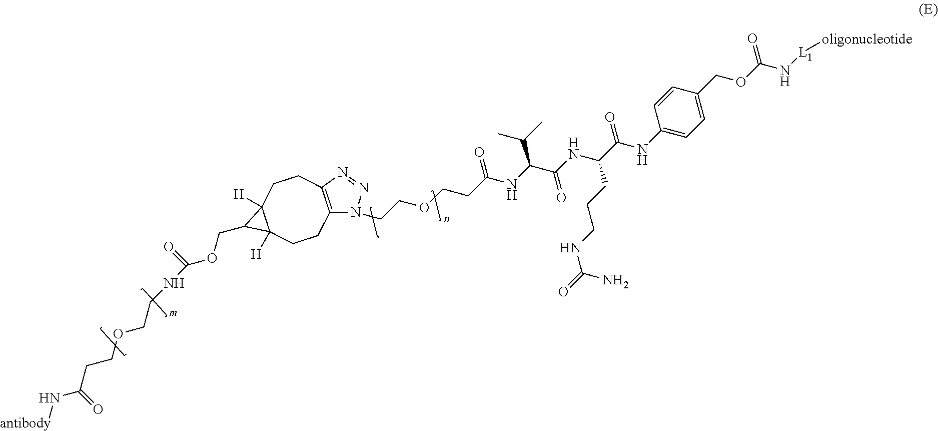

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine, the linker is linked to the oligonucleotide at the 5′ end, n is 3, and m is 4. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195). It should be understood that the amide shown adjacent the anti-TfR1 antibody in Formula (E) results from a reaction with an amine of the anti-TfR1 antibody, such as a lysine epsilon amine.

It should be appreciated that antibodies can be linked to molecular payloads with different stoichiometries, a property that may be referred to as a drug to antibody ratios (DAR) with the “drug” being the molecular payload. In some embodiments, one molecular payload is linked to an antibody (DAR=1). In some embodiments, two molecular payloads are linked to an antibody (DAR=2). In some embodiments, three molecular payloads are linked to an antibody (DAR=3). In some embodiments, four molecular payloads are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. An average DAR of complexes in a mixture need not be an integer value. DAR may be increased by conjugating molecular payloads to different sites on an antibody and/or (e.g., and) by conjugating multimers to one or more sites on antibody. For example, a DAR of 2 may be achieved by conjugating a single molecular payload to two different sites on an antibody or by conjugating a dimer molecular payload to a single site of an antibody.

In some embodiments, the complex described herein comprises an anti-TfR1 antibody described herein (e.g., the antibodies provided in Tables 2-7) covalently linked to a molecular payload. In some embodiments, the complex described herein comprises an anti-TfR1 antibody described herein (e.g., the antibodies provided in Tables 2-7) covalently linked to molecular payload via a linker (e.g., a linker comprising a valine-citrulline sequence). In some embodiments, the linker (e.g., a linker comprising a valine-citrulline sequence) is linked to the antibody (e.g., an anti-TfR1 antibody described herein) via a thiol-reactive linkage (e.g., via a cysteine in the antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a CDR-H1, a CDR-H2, a CDR-H3, a CDR-L1, a CDR-L2, and a CDR-L3 of any one of the antibodies listed in Table 2. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a VH comprising the amino acid sequence of SEQ ID NO: 69, SEQ ID NO: 71, or SEQ ID NO: 72, and a VL comprising the amino acid sequence of SEQ ID NO: 70. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 74. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 75. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a VH comprising the amino acid sequence of SEQ ID NO: 77, and a VL comprising the amino acid sequence of SEQ ID NO: 78. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a VH comprising the amino acid sequence of SEQ ID NO: 77 or SEQ ID NO: 79, and a VL comprising the amino acid sequence of SEQ ID NO: 80. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a VH comprising the amino acid sequence of SEQ ID NO: 154, and a VL comprising the amino acid sequence of SEQ ID NO: 155. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 84, SEQ ID NO: 86 or SEQ ID NO: 87 and a light chain comprising the amino acid sequence of SEQ ID NO: 85. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 89. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 90. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92 or SEQ ID NO: 94, and a light chain comprising the amino acid sequence of SEQ ID NO: 95. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92, and a light chain comprising the amino acid sequence of SEQ ID NO: 93. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 156, and a light chain comprising the amino acid sequence of SEQ ID NO: 157. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 97, SEQ ID NO: 98, or SEQ ID NO: 99 and a light chain comprising the amino acid sequence of SEQ ID NO: 85. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 89. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 90. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 and a light chain comprising the amino acid sequence of SEQ ID NO: 93. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 or SEQ ID NO: 103 and a light chain comprising the amino acid sequence of SEQ ID NO: 95. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In some embodiments, the complex described herein comprises an anti-TfR1 antibody covalently linked to a molecular payload, wherein the anti-TfR1 antibody comprises a heavy chain comprising the amino acid sequence of SEQ ID NO: 158 or SEQ ID NO: 159 and a light chain comprising the amino acid sequence of SEQ ID NO: 157. In some embodiments, the molecular payload is a DMD-targeting oligonucleotide (e.g., a DMD-targeting oligonucleotide listed in Table 8, provided by any one of SEQ ID NO: 196-267, or complementary to any one of SEQ ID NO: 160-195).

In any of the example complexes described herein, in some embodiments, the anti-TfR1 antibody is covalently linked to the molecular payload via a linker comprising a structure of:

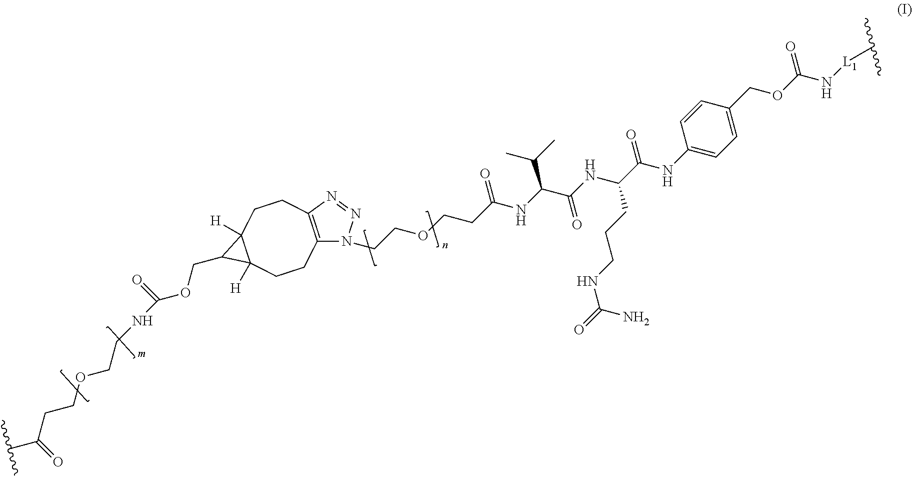

wherein n is 3, m is 4.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 196-267, or complementary to any one of SEQ ID NO: 160-195) via a lysine in the anti-TfR1 antibody, wherein the anti-TfR1 antibody comprises a CDR-H1, a CDR-H2, a CDR-H3, a CDR-L1, a CDR-L2, and a CDR-L3 of any one of the antibodies listed in Table 2, wherein the complex has a structure of:

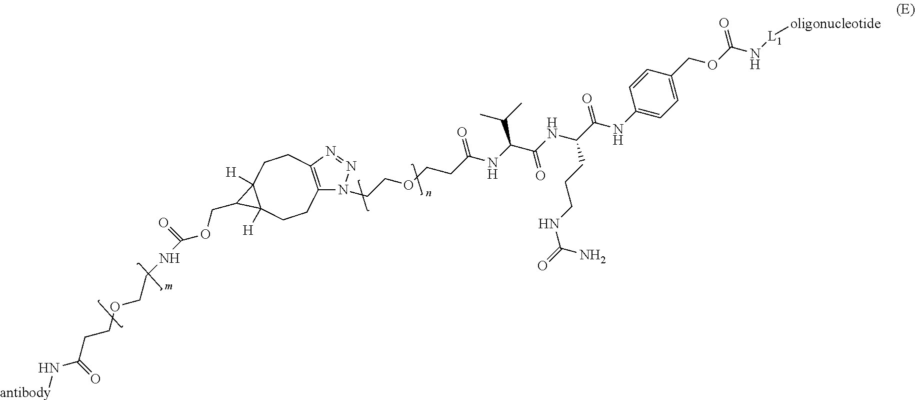

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 196-267, or complementary to any one of SEQ ID NO: 160-195) via a lysine in the anti-TfR1 antibody, wherein the anti-TfR1 antibody comprises a VH and VL of any one of the antibodies listed in Table 3, wherein the complex has a structure of:

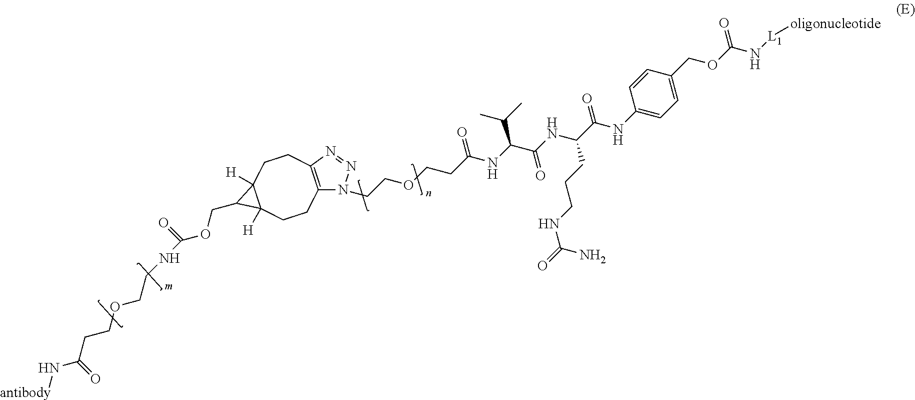

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 196-267, or complementary to any one of SEQ ID NO: 160-195) via a lysine in the anti-TfR1 antibody, wherein the anti-TfR1 antibody comprises a heavy chain and light chain of any one of the antibodies listed in Table 4, wherein the complex has a structure of:

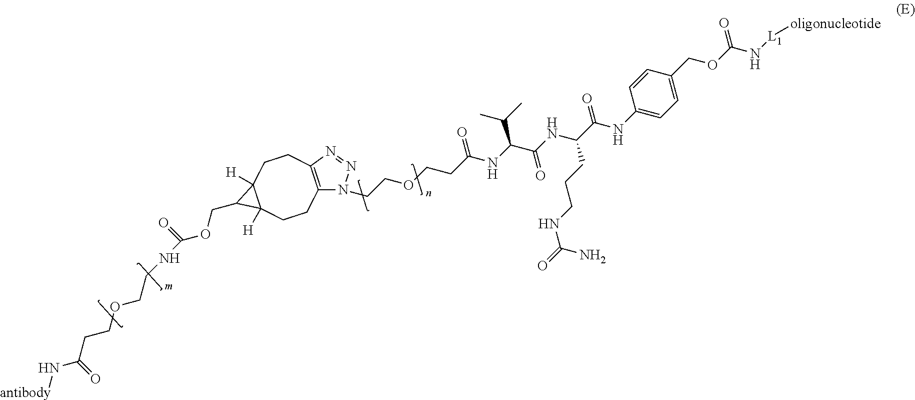

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 Fab covalently linked to the 5′ end of a DMD-targeting oligonucleotide (e.g., a DMD-targeting oligonucleotide listed in Table 8, provided by any one of SEQ ID NO: 196-267, or complementary to any one of SEQ ID NO: 160-195) via a lysine in the anti-TfR1 antibody, wherein the anti-TfR1 Fab comprises a heavy chain and light chain of any one of the antibodies listed in Table 5, wherein the complex has a structure of:

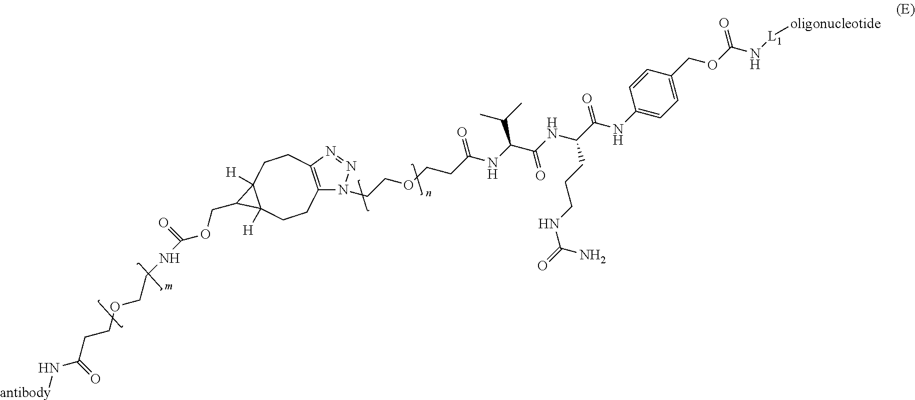

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, in any one of the examples of complexes described herein, L1 is:

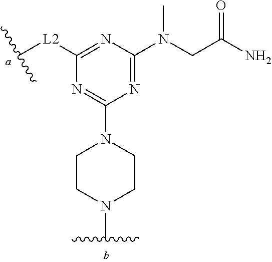

wherein L2 is

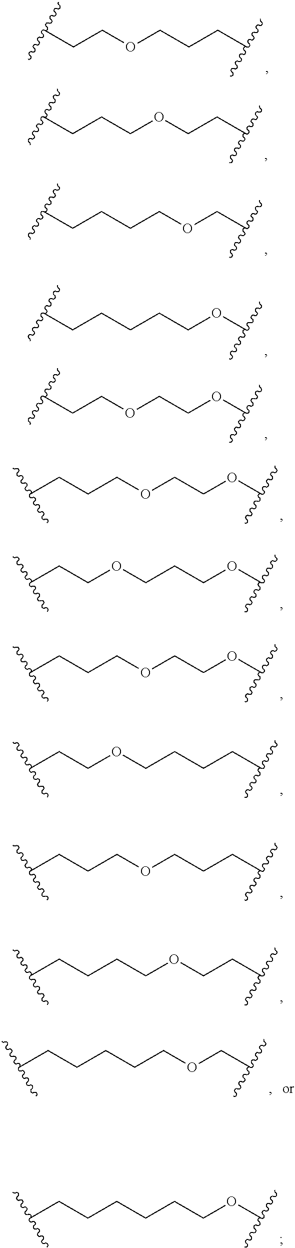

wherein a labels the site directly linked to the carbamate moiety of formulae (B), (D), (E), and (I); and b labels the site covalently linked (directly or via additional chemical moieties) to the oligonucleotide.

In some embodiments, L1 is:

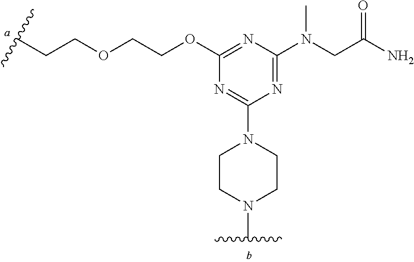 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, Li is linked to a 5′ phosphate of the oligonucleotide. In some embodiments, the phosphate is a phosphodiester. In some embodiments, L1 is linked to a 5′ phosphorothioate of the oligonucleotide. In some embodiments, Li is linked to a 5′ phosphonoamidate of the oligonucleotide. In some embodiments, L1 is linked via a phosphorodiamidate linkage to the 5′ end of the oligonucleotide.

In some embodiments, Li is optional (e.g., need not be present).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or (e.g., and) uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently linked to a molecular payload as described herein are effective in treating a subject having a dystrophinopathy, e.g., Duchenne muscular dystrophy. In some embodiments, complexes comprise a molecular payload that is an oligonucleotide, e.g., an antisense oligonucleotide that facilitates exon skipping of a pre-mRNA expressed from a mutated DMD allele.

In some embodiments, a subject may be a human subject, a non-human primate subject, a rodent subject, or any suitable mammalian subject. In some embodiments, a subject may have Duchenne muscular dystrophy or other dystrophinopathy. In some embodiments, a subject has a mutated DMD allele, which may optionally comprise at least one mutation in a DMD exon that causes a frameshift mutation and leads to improper RNA splicing/processing. In some embodiments, a subject is suffering from symptoms of a severe dystrophinopathy, e.g. muscle atrophy or muscle loss. In some embodiments, a subject has an asymptomatic increase in serum concentration of creatine phosphokinase (CK) and/or (e.g., and) muscle cramps with myoglobinuria. In some embodiments, a subject has a progressive muscle disease, such as Duchenne or Becker muscular dystrophy or DMD-associated dilated cardiomyopathy (DCM). In some embodiments, a subject is not suffering from symptoms of a dystrophinopathy.

In some embodiments, a subject has a mutation in a DMD gene that is amenable to exon 44 skipping. In some embodiments, a complex comprising a muscle-targeting agent covalently linked to a molecular payload as described herein is effective in treating a subject having a mutation in a DMD gene that is amenable to exon 44 skipping. In some embodiments, a complex comprises a molecular payload that is an oligonucleotide, e.g., an antisense oligonucleotide that facilitates skipping of exon 44 of a pre-mRNA, such as in a pre-mRNA encoded from a mutated DMD gene (e.g., a mutated DMD gene that is amenable to exon 44 skipping).

An aspect of the disclosure includes methods involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g., age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g., the half-life of the complex in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of observation of symptoms associated with a dystrophinopathy, e.g., muscle atrophy or muscle weakness, through measures of a subject's self-reported outcomes, e.g., mobility, self-care, usual activities, pain/discomfort, and anxiety/depression, or by quality-of-life indicators, e.g., lifespan.

In some embodiments, a pharmaceutical composition that comprises a complex comprising a muscle-targeting agent covalently linked to a molecular payload described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### Additional Embodiments

1. A complex comprising an anti-transferrin receptor 1 (TfR1) antibody covalently linked to a molecular payload configured for inducing skipping of exon 44 in a DMD pre-mRNA, wherein the anti-TfR1 antibody is an antibody identified in any one of Tables 2-7.
2. The complex of embodiment 1, wherein the anti-TfR1 antibody comprises:
(i) a heavy chain complementarity determining region 1 (CDR-H1) of SEQ ID NO: 33, a heavy chain complementarity determining region 2 (CDR-H2) of SEQ ID NO: 34, a heavy chain complementarity determining region 3 (CDR-H3) of SEQ ID NO: 35, a light chain complementarity determining region 1 (CDR-L1) of SEQ ID NO: 36, a light chain complementarity determining region 2 (CDR-L2) of SEQ ID NO: 37, and a light chain complementarity determining region 3 (CDR-L3) of SEQ ID NO: 32;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6;
(iii) a CDR-H1 of SEQ ID NO: 7, a CDR-H2 of SEQ ID NO: 20, a CDR-H3 of SEQ ID NO: 9, a CDR-L1 of SEQ ID NO: 10, a CDR-L2 of SEQ ID NO: 11, and a CDR-L3 of SEQ ID NO: 6;
(iv) a CDR-H1 of SEQ ID NO: 7, a CDR-H2 of SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6;
(v) a CDR-H1 of SEQ ID NO: 51, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50;
(vi) a CDR-H1 of SEQ ID NO: 64, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
(vii) a CDR-H1 of SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50.
   3. The complex of embodiment 1 or embodiment 2, wherein the anti-TfR1 antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a VH comprising an amino acid sequence at least 85% identical to SEQ ID NO: 69; and/or a VL comprising an amino acid sequence at least 85% identical to SEQ ID NO: 70;
(iii) a VH comprising an amino acid sequence at least 85% identical to SEQ ID NO: 71; and/or a VL comprising an amino acid sequence at least 85% identical to SEQ ID NO: 70;
(iv) a VH comprising an amino acid sequence at least 85% identical to SEQ ID NO: 72; and/or a VL comprising an amino acid sequence at least 85% identical to SEQ ID NO: 70;
(v) a VH comprising an amino acid sequence at least 85% identical to SEQ ID NO: 73; and/or a VL comprising an amino acid sequence at least 85% identical to SEQ ID NO: 74;
(vi) a VH comprising an amino acid sequence at least 85% identical to SEQ ID NO: 73; and/or a VL comprising an amino acid sequence at least 85% identical to SEQ ID NO: 75;
(vii) a VH comprising an amino acid sequence at least 85% identical to SEQ ID NO: 76; and/or a VL comprising an amino acid sequence at least 85% identical to SEQ ID NO: 74;
(viii) a VH comprising an amino acid sequence at least 85% identical to SEQ ID NO: 77; and/or a VL comprising an amino acid sequence at least 85% identical to SEQ ID NO: 78;
(ix) a VH comprising an amino acid sequence at least 85% identical to SEQ ID NO: 79; and/or a VL comprising an amino acid sequence at least 85% identical to SEQ ID NO: 80; or
(x) a VH comprising an amino acid sequence at least 85% identical to SEQ ID NO: 77; and/or a VL comprising an amino acid sequence at least 85% identical to SEQ ID NO: 80.
   4. The complex of any one of embodiments 1 to 3, wherein the anti-TfR1 antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 69 and a VL comprising the amino acid sequence of SEQ ID NO: 70;
(iii) a VH comprising the amino acid sequence of SEQ ID NO: 71 and a VL comprising the amino acid sequence of SEQ ID NO: 70;
(iv) a VH comprising the amino acid sequence of SEQ ID NO: 72 and a VL comprising the amino acid sequence of SEQ ID NO: 70;
(v) a VH comprising the amino acid sequence of SEQ ID NO: 73 and a VL comprising the amino acid sequence of SEQ ID NO: 74;
(vi) a VH comprising the amino acid sequence of SEQ ID NO: 73 and a VL comprising the amino acid sequence of SEQ ID NO: 75;
(vii) a VH comprising the amino acid sequence of SEQ ID NO: 76 and a VL comprising the amino acid sequence of SEQ ID NO: 74;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.
   5. The complex of any one of embodiments 1 to 4, wherein the anti-TfR1 antibody is a Fab fragment, a Fab′ fragment, a F(ab′)2 fragment, an scFv, an Fv, or a full-length IgG.
   6. The complex of embodiment 5, wherein the anti-TfR1 antibody is a Fab fragment.
   7. The complex of embodiment 6, wherein the anti-TfR1 antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 97; and/or a light chain comprising an amino acid sequence at least 85% identical to SEQ ID NO: 85;
(iii) a heavy chain comprising an amino acid sequence at least 85% identical to SEQ ID NO: 98; and/or a light chain comprising an amino acid sequence at least 85% identical to SEQ ID NO: 85;
(iv) a heavy chain comprising an amino acid sequence at least 85% identical to SEQ ID NO: 99; and/or a light chain comprising an amino acid sequence at least 85% identical to SEQ ID NO: 85;
(v) a heavy chain comprising an amino acid sequence at least 85% identical to SEQ ID NO: 100; and/or a light chain comprising an amino acid sequence at least 85% identical to SEQ ID NO: 89;
(vi) a heavy chain comprising an amino acid sequence at least 85% identical to SEQ ID NO: 100; and/or a light chain comprising an amino acid sequence at least 85% identical to SEQ ID NO: 90;
(vii) a heavy chain comprising an amino acid sequence at least 85% identical to SEQ ID NO: 101; and/or a light chain comprising an amino acid sequence at least 85% identical to SEQ ID NO: 89;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.
   8. The complex of embodiment 6 or embodiment 7, wherein the anti-TfR1 antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 97; and a light chain comprising the amino acid sequence of SEQ ID NO: 85;
(iii) a heavy chain comprising the amino acid sequence of SEQ ID NO: 98; and a light chain comprising the amino acid sequence of SEQ ID NO: 85;
(iv) a heavy chain comprising the amino acid sequence of SEQ ID NO: 99; and a light chain comprising the amino acid sequence of SEQ ID NO: 85;
(v) a heavy chain comprising the amino acid sequence of SEQ ID NO: 100; and a light chain comprising the amino acid sequence of SEQ ID NO: 89;
(vi) a heavy chain comprising the amino acid sequence of SEQ ID NO: 100; and a light chain comprising the amino acid sequence of SEQ ID NO: 90;
(vii) a heavy chain comprising the amino acid sequence of SEQ ID NO: 101; and a light chain comprising the amino acid sequence of SEQ ID NO: 89;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.
   9. The complex of any one of embodiments 1 to 8, wherein the anti-TfR1 antibody does not specifically bind to the transferrin binding site of the transferrin receptor 1 and/or wherein the anti-TfR1 antibody does not inhibit binding of transferrin to the transferrin receptor 1.
   10. The complex of any one of embodiments 1 to 9, wherein the molecular payload comprises an oligonucleotide.
   11. The complex of embodiment 10, wherein the oligonucleotide promotes antisense-mediated exon skipping in the DMD pre-RNA.
   12. The complex of embodiment 10 or 11, wherein the oligonucleotide comprises a region of complementarity to a splicing feature of the DMD pre-mRNA.
   13. The complex of embodiment 12, wherein the splicing feature is an exonic splicing enhancer (ESE) of the DMD pre-mRNA.
   14. The complex of embodiment 13, wherein the splicing feature is in exon 44 of the DMD pre-mRNA, optionally wherein the ESE comprises a sequence of any one of SEQ ID NOs: 286-296.
   15. The complex of embodiment 12, wherein the splicing feature is a branch point, a splice donor site, or a splice acceptor site.
   16. The complex of embodiment 15, wherein the splicing feature is across the junction of exon 43 and intron 43, in intron 43, across the junction of intron 43 and exon 44, across the junction of exon 44 and intron 44, in intron 44, or across the junction of intron 44 and exon 45 of the DMD pre-mRNA, optionally wherein the splicing feature comprises a sequence of any one of SEQ ID NOs: 282-285 and 297-301.
   17. The complex of any one of embodiments 12 to 16, wherein the region of complementarity comprises at least 4 consecutive nucleosides complementary to the splicing feature.
   18. The complex of any one of embodiments 1 to 9, wherein the molecular payload comprises an oligonucleotide comprising a sequence complementary to any one of SEQ ID NOs: 160-195 or comprising a sequence of any one of SEQ ID NOs: 196-267, wherein each thymine base (T) may independently and optionally be replaced with a uracil base (U), and each U may independently and optionally be replaced with a T.
   19. The complex of any one of embodiments 10 to 18, wherein the oligonucleotide comprises at least one modified internucleoside linkage.
   20. The complex of embodiment 19, wherein the at least one modified internucleoside linkage is a phosphorothioate linkage.
   21. The complex of any one of embodiments 10 to 20, wherein the oligonucleotide comprises one or more modified nucleosides.
   22. The complex of embodiment 21, wherein the one or more modified nucleosides are 2′-modified nucleosides.
   23. The complex of any one of embodiments 10 to 18, wherein the oligonucleotide comprises one or more phosphorodiamidate morpholinos, optionally wherein the oligonucleotide is a phosphorodiamidate morpholino oligomer (PMO).
   24. The complex of any one of embodiments 1 to 23, wherein the anti-TfR1 antibody is covalently linked to the molecular payload via a cleavable linker.
   25. The complex of embodiment 24, wherein the cleavable linker comprises a valine-citrulline sequence.
   26. The complex of any one of embodiments 1 to 25, wherein the anti-TfR1 antibody is covalently linked to the molecular payload via conjugation to a lysine residue or a cysteine residue of the antibody.
   27. A complex comprising an anti-TfR1 antibody covalently linked to an oligonucleotide configured for inducing skipping of exon 44 in a DMD pre-mRNA, wherein the oligonucleotide comprises a region of complementarity to any one of SEQ ID NOs: 160-195.
   28. The complex of embodiment 27, wherein the anti-TfR1 antibody is an antibody identified in any one of Tables 2-7.
   29. A complex comprising an anti-TfR1 antibody covalently linked to an oligonucleotide configured for inducing skipping of exon 44 in a DMD pre-mRNA, wherein the oligonucleotide comprises a region of complementarity to a splicing feature of the DMD pre-mRNA.
   30. An oligonucleotide that targets DMD, wherein the oligonucleotide comprises a region of complementarity to any one of SEQ ID NOs: 160-195.
   31. The oligonucleotide of embodiment 30, wherein the region of complementarity comprises at least 15 consecutive nucleosides complementary to any one of SEQ ID NOs: 160-195.
   32. The oligonucleotide of embodiment 30 or 31, wherein the oligonucleotide comprises at least 15 consecutive nucleosides of any one of SEQ ID NOs: 196-267, optionally wherein the oligonucleotide comprises a sequence of any one of SEQ ID NOs: 196-267, wherein each thymine base (T) may independently and optionally be replaced with a uracil base (U), and each U may independently and optionally be replaced with a T.
   33. A method of delivering a molecular payload to a cell, the method comprising contacting the cell with the complex of any one of embodiments 1 to 26.
   34. A method of delivering an oligonucleotide to a cell, the method comprising contacting the cell with the complex of any one of embodiments 27 to 29.
   35. A method of promoting the expression or activity of a dystrophin protein in a cell, the method comprising contacting the cell with the complex of any one of embodiments 1 to 26 in an amount effective for promoting internalization of the molecular payload to the cell, optionally wherein the cell is a muscle cell.
   36. A method of promoting the expression or activity of a dystrophin protein in a cell, the method comprising contacting the cell with the complex of any one of embodiments 27 to 29 in an amount effective for promoting internalization of the oligonucleotide to the cell, optionally wherein the cell is a muscle cell.
   37. The method of embodiment 35 or 36, wherein the cell is in vitro.
   38. The method of embodiment 35 or 36, wherein the cell is in a subject.
   39. The method of embodiment 38, wherein the subject is a human.
   40. The method of embodiment 39, wherein the subject has a DMD gene that is amenable to skipping of exon 44.
   41. The method of any one of embodiments 35 to 40, wherein the dystrophin protein is a truncated dystrophin protein.
   42. A method of treating a subject having a mutated DMD allele that is associated with a dystrophinopathy, the method comprising administering to the subject an effective amount of the complex of any one of embodiments 1 to 29.
   43. A method of promoting skipping of exon 44 of a DMD pre-mRNA transcript in a cell, the method comprising contacting the cell with an effective amount of the complex of any one of embodiments 1 to 29.
   44. A method of treating a subject having a mutated DMD allele that is associated with a dystrophinopathy, the method comprising administering to the subject an effective amount of the complex of any one of embodiments 1 to 29.

### Examples

### Example 1. Exon-Skipping Activity of Anti-TfR1 Antibody Conjugates in Duchenne Muscular Dystrophy Patient Myotubes

In this study, the exon-skipping activities of anti-TfR1 antibody conjugates comprising an anti-TfR1 Fab (3M12 VH4/Vκ3) covalently linked to a DMD exon 51-skipping antisense oligonucleotide (ASO) were evaluated. The DMD exon 51-skipping ASO is a phosphorodiamidate morpholino oligomer (PMO) of 30 nucleotides in length and targets an ESE in DMD exon 51 having the sequence TGGAGGT (SEQ ID NO: 322). Immortalized human myoblasts bearing an exon 52 deletion in the DMD gene were thawed and seeded at a density of 1e6 cell/flask in Promocell Skeletal Cell Growth Media (with 5% FBS and 1× Pen-Strep) and allowed to grow to confluency. Once confluent, cells were trypsinized and pelleted via centrifugation and resuspended in fresh Promocell Skeletal Cell Growth Media. The cell number was counted and cells were seeded into Matrigel-coated 96-well plates at a density of 50,000 cells/well. Cells were allowed to recover for 24 hours. Cells were induced to differentiate into myotubes by aspirating the growth media and replacing with differentiation media with no serum. Cells were then treated with the DMD exon 51-skipping oligonucleotide (not covalently linked to an antibody—“naked”) at 10 μM ASO or the anti-TfR1 Fab (3M12 VH4/Vκ3) covalently linked to the DMD exon 51-skipping oligonucleotide at 10 μM ASO equivalent. Cells were incubated with test articles for ten days then total RNA was harvested from the 96 well plates. cDNA synthesis was performed on 75 ng of total RNA, and mutation specific PCRs were performed to evaluate the degree of exon 51 skipping in the cells. Mutation-specific PCR products were run on a 4% agarose gel and visualized using SYBR gold. Densitometry was used to calculate the relative amounts of the skipped and unskipped amplicon and exon skipping was determined as a ratio of the Exon 51 skipped amplicon divided by the total amount of amplicon present:

$$\% Exon Skipping = \frac{Skipped Amplicon}{( Skipped Amplicon + U n s k ipped Amplicon )} * 100.$$

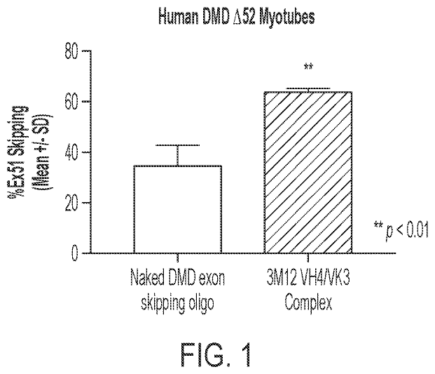

The results demonstrate that the conjugate resulted in enhanced exon skipping compared to the naked DMD exon 51-skipping oligonucleotide in patient myotubes (FIG. 1). This indicates that anti-TfR1 Fab 3M12 VH4/Vκ3 enabled cellular internalization of the conjugate into muscle cells resulting in activity of the exon 51-skipping oligonucleotide in the muscle cells. Similarly, an anti-TfR1 antibody (e.g., anti-TfR1 Fab 3M12 VH4/Vκ3) can enable internalization of a conjugate comprising the anti-TfR1 antibody covalently linked to other exon skipping oligonucleotides (e.g., an exon skipping oligonucleotide provided herein, such as an exon 44 skipping oligonucleotide) into muscle cells and facilitate activity of the exon skipping oligonucleotide in the muscle cells.

### Example 2. Exon Skipping Activity of Anti-TfR1 Fab-ASO Conjugate In Vivo in Cynomolgus Monkeys

Anti-TfR1 Fab 3M12 VH4/Vκ3 was covalently linked to the DMD exon 51-skipping antisense oligonucleotide (ASO) that was used in Example 1. The exon skipping activity of the conjugate was tested in vivo in healthy non-human primates. Naïve male cynomolgus monkeys (n=4-5 per group) were administered two doses of vehicle, 30 mg/kg naked ASO (i.e., not covalently linked to an antibody), or 122 mg/kg anti-TfR1 Fab (3M12 VH4/Vκ3) covalently linked to the DMD exon 51-skipping oligonucleotide (30 mg/kg ASO equivalent) via intravenous infusion on days 1 and 8. Animals were sacrificed and tissues harvested either 2 weeks or 4 weeks after the first dose was administered. Total RNA was collected from tissue samples using a Promega Maxwell® RSC instrument and cDNA synthesis was performed using qScript cDNA SuperMix. Assessment of exon 51 skipping was performed using end-point PCR.

Capillary electrophoresis of the PCR products was used to assess exon skipping, and % exon 51 skipping was calculated using the following formula:

$$\% Exon Skipping = \frac{Molarity of Skipped Band}{Molarity of Skipped Band + Molarity of Unskipped Band} * 100 .$$

Calculated exon 51 skipping results are shown in Table 10.

TABLE 10
Exon 51 skipping of DMD mRNA in cynomolgus monkey

| | Time | | | | |
|---|---|---|---|---|---|
| | 2 weeks | | | 4 weeks | |
| | | Naked | | Naked | |
| Group | Vehicle | ASO^{a} | Conjugate | ASO^{a} | Conjugate |
| Conjugate dose^{b} | 0 | n/a | 122 | n/a | 122 |
| ASO Dose^{c} | 0 | 30 | 30 | 30 | 30 |
| Quadriceps ^{d} | 0.00 | 1.216 | 4.906 | 0.840 | 1.708 |
| | (0.00) | (1.083) | (3.131) | (1.169) | (1.395) |
| Diaphragm ^{d} | 0.00 | 1.891 | 7.315 | 0.717 | 9.225 |
| | (0.00) | (2.911) | (1.532) | (1.315) | (4.696) |
| Heart ^{d} | 0.00 | 0.043 | 3.42 | 0.00 | 4.525 |
| | (0.00) | (0.096) | (1.192) | (0.00) | (1.400) |
| Biceps ^{d} | 0.00 | 0.607 | 3.129 | 1.214 | 4.863 |
| | (0.00) | (0.615) | (0.912) | (1.441) | (3.881) |
| Tibialis | 0.00 | 0.699 | 1.042 | 0.384 | 0.816 |
| anterior ^{d} | (0.00) | (0.997) | (0.685) | (0.615) | (0.915) |
| Gastrocnemius ^{d} | 0.00 | 0.388 | 2.424 | 0.00 | 5.393 |
| | (0.00) | (0.573) | (2.329) | (0.00) | (2.695) |

^{a}ASO = antisense oligonucleotide.
^{b}Conjugate doses are listed as mg/kg of anti-TfR1 Fab 3M12 VH4/Vκ3-ASO conjugate.
^{c}ASO doses are listed as mg/kg ASO or ASO equivalent of the anti-TfR1 Fab 3M12 VH4/Vκ3-ASO dose.
^{d} Exon skipping values are mean % exon 51 skipping with standard deviations (n = 5) in parentheses.

Tissue ASO accumulation was also quantified using a hybridization ELISA with a probe complementary to the ASO sequence. A standard curve was generated and ASO levels (in ng/g) were derived from a linear regression of the standard curve. The ASO was distributed to all tissues evaluated at a higher level following the administration of the anti-TfR1 Fab VH4/Vκ3-ASO conjugate as compared to the administration of naked ASO. Intravenous administration of naked ASO resulted in levels of ASO that were close to background levels in all tissues evaluated at 2 and 4 weeks after the first does was administered. Administration of anti-TfR1 Fab VH4/Vκ3-ASO conjugate resulted in distribution of ASO through the tissues evaluated with a rank order of heart>diaphragm>bicep>quadriceps>gastrocnemius>tibialis anterior 2 weeks after first dosing. The duration of tissue concentration was also assessed. Concentrations of the ASO in quadriceps, bicep and diaphragm decreased by less than 50% over the time period evaluated (2 to 4 weeks), while levels of ASO in the heart, tibialis anterior, and gastrocnemius remained virtually unchanged (Table 11). This indicates that anti-TfR1 Fab 3M12 VH4/Vκ3 enabled cellular internalization of the conjugate into muscle cells in vivo, resulting in activity of the exon skipping oligonucleotide in the muscle cells. Similarly, an anti-TfR1 antibody (e.g., anti-TfR1 Fab 3M12 VH4/Vκ3) in vivo can enable internalization of a conjugate comprising the anti-TfR1 antibody covalently linked to other exon skipping oligonucleotides (e.g., an exon skipping oligonucleotide provided herein, such as an exon 44 skipping oligonucleotide) into muscle cells and facilitate activity of the exon skipping oligonucleotide in the muscle cells.

TABLE 11
Tissue distribution of DMD exon 51
skipping ASO in cynomolgus monkeys

| | Time | | | | |
|---|---|---|---|---|---|
| | 2 weeks | | | 4 weeks | |
| | | Naked | Conju- | Naked | Conju- |
| Group | Vehicle | ASO^{a} | gate | ASO^{a} | gate |
| Conjugate Dose^{b} | 0 | n/a | 122 | n/a | 122 |
| ASO Dose^{c} | 0 | 30 | 30 | 30 | 30 |
| Quadriceps ^{d} | 0 | 696.8 | 2436 | 197 | 682 |
| | (59.05) | (868.15) | (954.0) | (134) | (281) |
| Diaphragm ^{d} | 0± | 580.02 | 6750 | 60 | 3131 |
| | (144.3) | (360.11) | (2256) | (120) | (1618) |
| Heart ^{d} | 0 | 1449 | 27138 | 943 | 30410 |
| | (396.03) | (1337) | (6315) | (1803) | (9247) |
| Biceps ^{d} | 0 | 615.63 | 2840 | 130 | 1326 |
| | (69.58) | (335.17) | (980.31) | (80) | (623) |
| Tibialis | 0 | 564.71 | 1591 | 169 | 1087 |
| anterior ^{d} | (76.31) | (327.88) | (253.50) | (110) | (514) |
| Gastrocnemius ^{d} | 0 | 705.47 | 2096 | 170 | 1265 |
| | (41.15) | (863.75) | (474.04) | (69) | (272) |

^{a}ASO = Antisense oligonucleotide.
^{b}Conjugate doses are listed as mg/kg of anti-TfR1 Fab 3M12 VH4/Vκ3-ASO conjugate.
^{c}ASO doses are listed as mg/kg ASO or ASO equivalent of the anti-TfR1 Fab 3M12 VH4/Vκ3-ASO conjugate dose.
^{d} ASO values are mean concentrations of ASO in tissue as ng/g with standard deviations (n = 5) in parentheses.

### Example 3. Exon-Skipping Activity of Anti-TfR1 Antibody Conjugates in Duchenne Muscular Dystrophy Patient Myotubes

In this study, the exon-skipping activities of anti-TfR1 antibody conjugates comprising an anti-TfR1 Fab (3M12 VH4/Vκ3) covalently linked to a DMD exon 44-skipping antisense oligonucleotide (ASO) are evaluated. The DMD exon 44-skipping ASO is a phosphorodiamidate morpholino oligomer (PMO) and targets a DMD exon 44 splicing feature. Immortalized human myoblasts are thawed and seeded at a density of 1e6 cell/flask in Promocell Skeletal Cell Growth Media (with 5% FBS and 1× Pen-Strep) and allowed to grow to confluency. Once confluent, cells are trypsinized and pelleted via centrifugation and resuspended in fresh Promocell Skeletal Cell Growth Media. The cell number is counted and cells are seeded into Matrigel-coated 96-well plates at a density of 50,000 cells/well. Cells are allowed to recover for 24 hours. Cells are induced to differentiate into myotubes by aspirating the growth media and replacing with differentiation media with no serum. Cells are then treated with the DMD exon 44-skipping oligonucleotide (not covalently linked to an antibody—“naked”) at 10 μM ASO or the anti-TfR1 Fab (3M12 VH4/Vκ3) covalently linked to the DMD exon 44-skipping oligonucleotide at 10 μM ASO equivalent. Cells are incubated with test articles for ten days then total RNA is harvested from the 96 well plates. cDNA synthesis is performed on 75 ng of total RNA, and mutation specific PCRs are performed to evaluate the degree of exon 44 skipping in the cells. PCR products are measured using capillary electrophoresis with UV detection. Molarity is calculated and relative amounts of the skipped and unskipped amplicon are determined. Exon skipping is determined as a ratio of the Exon 44 skipped amplicon divided by the total amount of amplicon present, according to the following formula:

$$\% Exon Skipping = \frac{Skipped Amplicon}{( Skipped Amplicon + U n s k ipped Amplicon )} * 100$$

The results demonstrate that the conjugates facilitate enhanced exon skipping compared to the naked DMD exon 44-skipping oligonucleotide in patient myotubes. This indicates that anti-TfR1 Fab 3M12 VH4/Vκ3 enables cellular internalization of the conjugate into muscle cells resulting in activity of the exon 44-skipping oligonucleotide in the muscle cells.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides or nucleosides (e.g., an RNA counterpart of a DNA nucleoside or a DNA counterpart of an RNA nucleoside) and/or (e.g., and) one or more modified nucleotides/nucleosides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A complex comprising an anti-transferrin receptor 1 (TfR1) antibody covalently linked to an oligonucleotide configured for inducing skipping of exon 44 in a DMD pre-mRNA, wherein the oligonucleotide comprises a region of complementarity that is complementary with at least 8 consecutive nucleotides of any one of SEQ ID NOs: 160-195.

2.-4. (canceled)

5. The complex of claim 1, wherein the anti-TfR1 antibody is a Fab fragment, a Fab′ fragment, a F(ab′)2 fragment, an scFv, an Fv, or a full-length IgG.

6. The complex of claim 5, wherein the anti-TfR1 antibody is a Fab fragment.

7.-8. (canceled)

9. The complex of claim 1, wherein the anti-TfR1 antibody does not specifically bind to the transferrin binding site of the transferrin receptor 1 and/or wherein the anti-TfR1 antibody does not inhibit binding of transferrin to the transferrin receptor 1.

10. The complex of claim 1, wherein the oligonucleotide comprises a region of complementarity to at least 4 consecutive nucleotides of a splicing feature of the DMD pre-mRNA.

11. The complex of claim 10, wherein the splicing feature is an exonic splicing enhancer (ESE) in exon 44 of the DMD pre-mRNA, optionally wherein the ESE comprises a sequence of any one of SEQ ID NOs: 286-296.

12. The complex of claim 10, wherein the splicing feature is a branch point, a splice donor site, or a splice acceptor site, optionally wherein the splicing feature is across the junction of exon 43 and intron 43, in intron 43, across the junction of intron 43 and exon 44, across the junction of exon 44 and intron 44, in intron 44, or across the junction of intron 44 and exon 45 of the DMD pre-mRNA, and further optionally wherein the splicing feature comprises a sequence of any one of SEQ ID NOs: 282-285 and 297-301.

13. The complex of claim 1, wherein the oligonucleotide comprises a sequence complementary to any one of SEQ ID NOs: 160-195 or comprises a sequence of any one of SEQ ID NOs: 196-267, wherein each thymine base (T) may independently and optionally be replaced with a uracil base (U), and each U may independently and optionally be replaced with a T.

14. The complex of claim 1, wherein the oligonucleotide comprises one or more phosphorodiamidate morpholinos, optionally wherein the oligonucleotide is a phosphorodiamidate morpholino oligomer (PMO).

15. The complex of claim 1, wherein the anti-TfR1 antibody is covalently linked to the oligonucleotide via a cleavable linker, optionally wherein the cleavable linker comprises a valine-citrulline sequence.

16. The complex of claim 1, wherein the anti-TfR1 antibody is covalently linked to the oligonucleotide via conjugation to a lysine residue or a cysteine residue of the antibody.

17. An oligonucleotide that targets DMD, wherein the oligonucleotide comprises a region of complementarity to any one of SEQ ID NOs: 160-195, optionally wherein the region of complementarity comprises at least 15 consecutive nucleosides complementary to any one of SEQ ID NOs: 160-195.

18. The oligonucleotide of claim 17, wherein the oligonucleotide comprises at least 15 consecutive nucleosides of any one of SEQ ID NOs: 196-267, optionally wherein the oligonucleotide comprises a sequence of any one of SEQ ID NOs: 196-267, wherein each thymine base (T) may independently and optionally be replaced with a uracil base (U), and each U may independently and optionally be replaced with a T.

19. A method of delivering an oligonucleotide to a cell, the method comprising contacting the cell with the complex of claim 1.

20. A method of promoting the expression or activity of a dystrophin protein in a cell, the method comprising contacting the cell with the complex of claim 1 in an amount effective for promoting internalization of the oligonucleotide to the cell, optionally wherein the cell is a muscle cell.


SEQUENCE LISTING (355 sequences)

SEQ ID NO: 1 - protein, 8 aa, synthetic construct
  REGION 1..8: note = Synthetic
GFNIKDDY

SEQ ID NO: 2 - protein, 8 aa, synthetic construct
  REGION 1..8: note = Synthetic
IDPENGDT

SEQ ID NO: 3 - protein, 10 aa, synthetic construct
  REGION 1..10: note = Synthetic
TLWLRRGLDY

SEQ ID NO: 4 - protein, 11 aa, synthetic construct
  REGION 1..11: note = Synthetic
KSLLHSNGYTY

SEQ ID NO: 5 - skipped

SEQ ID NO: 6 - protein, 9 aa, synthetic construct
  REGION 1..9: note = Synthetic
MQHLEYPFT

SEQ ID NO: 7 - protein, 5 aa, synthetic construct
  REGION 1..5: note = Synthetic
DDYMY

SEQ ID NO: 8 - protein, 17 aa, synthetic construct
  REGION 1..17: note = Synthetic
WIDPENGDTEYASKFQD

SEQ ID NO: 9 - protein, 8 aa, synthetic construct
  REGION 1..8: note = Synthetic
WLRRGLDY

SEQ ID NO: 10 - protein, 16 aa, synthetic construct
  REGION 1..16: note = Synthetic
RSSKSLLHSNGYTYLF

SEQ ID NO: 11 - protein, 7 aa, synthetic construct
  REGION 1..7: note = Synthetic
RMSNLAS

SEQ ID NO: 12 - protein, 7 aa, synthetic construct
  REGION 1..7: note = Synthetic
GFNIKDD

SEQ ID NO: 13 - skipped

SEQ ID NO: 14 - protein, 6 aa, synthetic construct
  REGION 1..6: note = Synthetic
LRRGLD

SEQ ID NO: 15 - protein, 12 aa, synthetic construct
  REGION 1..12: note = Synthetic
SKSLLHSNGYTY

SEQ ID NO: 16 - protein, 6 aa, synthetic construct
  REGION 1..6: note = Synthetic
HLEYPF

SEQ ID NO: 17 - protein, 117 aa, synthetic construct
  REGION 1..117: note = Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSS

SEQ ID NO: 18 - protein, 112 aa, synthetic construct
  REGION 1..112: note = Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIK

SEQ ID NO: 19 - protein, 8 aa, synthetic construct
  REGION 1..8: note = Synthetic
IDPETGDT

SEQ ID NO: 20 - protein, 17 aa, synthetic construct
  REGION 1..17: note = Synthetic
WIDPETGDTEYASKFQD

SEQ ID NO: 21 - skipped

SEQ ID NO: 22 - protein, 117 aa, synthetic construct
  REGION 1..117: note = Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSS

SEQ ID NO: 23 - protein, 8 aa, synthetic construct
  REGION 1..8: note = Synthetic
IDPESGDT

SEQ ID NO: 24 - protein, 17 aa, synthetic construct
  REGION 1..17: note = Synthetic
WIDPESGDTEYASKFQD

SEQ ID NO: 25 - skipped

SEQ ID NO: 26 - protein, 117 aa, synthetic construct
  REGION 1..117: note = Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSS

SEQ ID NO: 27 - protein, 9 aa, synthetic construct
  REGION 1..9: note = Synthetic
GYSITSGYY

SEQ ID NO: 28 - protein, 7 aa, synthetic construct
  REGION 1..7: note = Synthetic
ITFDGAN

SEQ ID NO: 29 - protein, 12 aa, synthetic construct
  REGION 1..12: note = Synthetic
TRSSYDYDVLDY

SEQ ID NO: 30 - protein, 6 aa, synthetic construct
  REGION 1..6: note = Synthetic
QDISNF

SEQ ID NO: 31 - skipped

SEQ ID NO: 32 - protein, 9 aa, synthetic construct
  REGION 1..9: note = Synthetic
QQGHTLPYT

SEQ ID NO: 33 - protein, 6 aa, synthetic construct
  REGION 1..6: note = Synthetic
SGYYWN

SEQ ID NO: 34 - protein, 16 aa, synthetic construct
  REGION 1..16: note = Synthetic
YITFDGANNYNPSLKN

SEQ ID NO: 35 - protein, 10 aa, synthetic construct
  REGION 1..10: note = Synthetic
SSYDYDVLDY

SEQ ID NO: 36 - protein, 11 aa, synthetic construct
  REGION 1..11: note = Synthetic
RASQDISNFLN

SEQ ID NO: 37 - protein, 7 aa, synthetic construct
  REGION 1..7: note = Synthetic
YTSRLHS

SEQ ID NO: 38 - protein, 8 aa, synthetic construct
  REGION 1..8: note = Synthetic
GYSITSGY

SEQ ID NO: 39 - skipped

SEQ ID NO: 40 - protein, 8 aa, synthetic construct
  REGION 1..8: note = Synthetic
SYDYDVLD

SEQ ID NO: 41 - protein, 7 aa, synthetic construct
  REGION 1..7: note = Synthetic
SQDISNF

SEQ ID NO: 42 - protein, 6 aa, synthetic construct
  REGION 1..6: note = Synthetic
GHTLPY

SEQ ID NO: 43 - protein, 119 aa, synthetic construct
  REGION 1..119: note = Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSS

SEQ ID NO: 44 - protein, 107 aa, synthetic construct
  REGION 1..107: note = Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIK

SEQ ID NO: 45 - protein, 8 aa, synthetic construct
  REGION 1..8: note = Synthetic
GYSFTDYC

SEQ ID NO: 46 - protein, 8 aa, synthetic construct
  REGION 1..8: note = Synthetic
IYPGSGNT

SEQ ID NO: 47 - protein, 13 aa, synthetic construct
  REGION 1..13: note = Synthetic
AREDYYPYHGMDY

SEQ ID NO: 48 - protein, 10 aa, synthetic construct
  REGION 1..10: note = Synthetic
ESVDGYDNSF

SEQ ID NO: 49 - skipped

SEQ ID NO: 50 - protein, 9 aa, synthetic construct
  REGION 1..9: note = Synthetic
QQSSEDPWT

SEQ ID NO: 51 - protein, 5 aa, synthetic construct
  REGION 1..5: note = Synthetic
DYCIN

SEQ ID NO: 52 - protein, 17 aa, synthetic construct
  REGION 1..17: note = Synthetic
WIYPGSGNTRYSERFKG

SEQ ID NO: 53 - protein, 11 aa, synthetic construct
  REGION 1..11: note = Synthetic
EDYYPYHGMDY

SEQ ID NO: 54 - protein, 15 aa, synthetic construct
  REGION 1..15: note = Synthetic
RASESVDGYDNSFMH

SEQ ID NO: 55 - protein, 7 aa, synthetic construct
  REGION 1..7: note = Synthetic
RASNLES

SEQ ID NO: 56 - protein, 7 aa, synthetic construct
  REGION 1..7: note = Synthetic
GYSFTDY

SEQ ID NO: 57 - skipped

SEQ ID NO: 58 - protein, 9 aa, synthetic construct
  REGION 1..9: note = Synthetic
DYYPYHGMD

SEQ ID NO: 59 - protein, 11 aa, synthetic construct
  REGION 1..11: note = Synthetic
SESVDGYDNSF

SEQ ID NO: 60 - protein, 6 aa, synthetic construct
  REGION 1..6: note = Synthetic
SSEDPW

SEQ ID NO: 61 - protein, 120 aa, synthetic construct
  REGION 1..120: note = Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 62 - protein, 111 aa, synthetic construct
  REGION 1..111: note = Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIK

SEQ ID NO: 63 - protein, 8 aa, synthetic construct
  REGION 1..8: note = Synthetic
GYSFTDYY

SEQ ID NO: 64 - protein, 5 aa, synthetic construct
  REGION 1..5: note = Synthetic
DYYIN

SEQ ID NO: 65 - protein, 120 aa, synthetic construct
  REGION 1..120: note = Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 66 - protein, 8 aa, synthetic construct
  REGION 1..8: note = Synthetic
GYSFTDYD

SEQ ID NO: 67 - protein, 5 aa, synthetic construct
  REGION 1..5: note = Synthetic
DYDIN

SEQ ID NO: 68 - protein, 120 aa, synthetic construct
  REGION 1..120: note = Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 69 - protein, 117 aa, synthetic construct
  REGION 1..117: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSS

SEQ ID NO: 70 - protein, 112 aa, synthetic construct
  REGION 1..112: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIK

SEQ ID NO: 71 - protein, 117 aa, synthetic construct
  REGION 1..117: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSS

SEQ ID NO: 72 - protein, 117 aa, synthetic construct
  REGION 1..117: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSS

SEQ ID NO: 73 - protein, 119 aa, synthetic construct
  REGION 1..119: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSS

SEQ ID NO: 74 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIK

SEQ ID NO: 75 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 76 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 77 - protein, 120 aa, synthetic construct
  REGION 1..120: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 78 - protein, 111 aa, synthetic construct
  REGION 1..111: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIK

SEQ ID NO: 79 - protein, 120 aa, synthetic construct
  REGION 1..120: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 80 - protein, 111 aa, synthetic construct
  REGION 1..111: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIK

SEQ ID NO: 81 - protein, 330 aa, Homo sapiens [sequence omitted]

SEQ ID NO: 82 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 107 aa, synthetic construct [sequence omitted]

SEQ ID NO: 84 - protein, 447 aa, synthetic construct
  REGION 1..447: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 219 aa, synthetic construct
  REGION 1..219: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 447 aa, synthetic construct
  REGION 1..447: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 447 aa, synthetic construct
  REGION 1..447: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 449 aa, synthetic construct
  REGION 1..449: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 449 aa, synthetic construct
  REGION 1..449: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 450 aa, synthetic construct
  REGION 1..450: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 218 aa, synthetic construct
  REGION 1..218: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 450 aa, synthetic construct
  REGION 1..450: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 218 aa, synthetic construct
  REGION 1..218: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 108 aa, synthetic construct
  REGION 1..108: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - protein, 225 aa, synthetic construct
  REGION 1..225: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 98 - protein, 225 aa, synthetic construct
  REGION 1..225: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 99 - protein, 225 aa, synthetic construct
  REGION 1..225: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 100 - protein, 227 aa, synthetic construct
  REGION 1..227: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 101 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 102 - protein, 228 aa, synthetic construct
  REGION 1..228: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 103 - protein, 228 aa, synthetic construct
  REGION 1..228: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 104 - protein, 19 aa, synthetic construct
  REGION 1..19: note = Synthetic
MGWSCIILFLVATATGVHS

SEQ ID NO: 105 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 106 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 107 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 108 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 109 - protein, 197 aa, synthetic construct
  REGION 1..197: note = Synthetic
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 110 - protein, 5 aa, synthetic construct
  REGION 1..5: note = Synthetic
SYWMH

SEQ ID NO: 111 - protein, 17 aa, synthetic construct
  REGION 1..17: note = Synthetic
EINPTNGRTNYIEKFKS

SEQ ID NO: 112 - protein, 7 aa, synthetic construct
  REGION 1..7: note = Synthetic
GTRAYHY

SEQ ID NO: 113 - protein, 11 aa, synthetic construct
  REGION 1..11: note = Synthetic
RASDNLYSNLA

SEQ ID NO: 114 - protein, 7 aa, synthetic construct
  REGION 1..7: note = Synthetic
DATNLAD

SEQ ID NO: 115 - protein, 9 aa, synthetic construct
  REGION 1..9: note = Synthetic
QHFWGTPLT

SEQ ID NO: 116 - protein, 7 aa, synthetic construct
  REGION 1..7: note = Synthetic
GYTFTSY

SEQ ID NO: 117 - protein, 6 aa, synthetic construct
  REGION 1..6: note = Synthetic
NPTNGR

SEQ ID NO: 118 - protein, 6 aa, synthetic construct
  REGION 1..6: note = Synthetic
TSYWMH

SEQ ID NO: 119 - protein, 13 aa, synthetic construct
  REGION 1..13: note = Synthetic
WIGEINPTNGRTN

SEQ ID NO: 120 - protein, 6 aa, synthetic construct
  REGION 1..6: note = Synthetic
ARGTRA

SEQ ID NO: 121 - protein, 7 aa, synthetic construct
  REGION 1..7: note = Synthetic
YSNLAWY

SEQ ID NO: 122 - protein, 10 aa, synthetic construct
  REGION 1..10: note = Synthetic
LLVYDATNLA

SEQ ID NO: 123 - protein, 8 aa, synthetic construct
  REGION 1..8: note = Synthetic
QHFWGTPL

SEQ ID NO: 124 - protein, 116 aa, Mus musculus
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 125 - protein, 107 aa, Mus musculus
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 126 - protein, 9 aa, synthetic construct
  REGION 1..9: note = Synthetic
QHFAGTPLT

SEQ ID NO: 127 - protein, 8 aa, synthetic construct
  REGION 1..8: note = Synthetic
QHFAGTPL

SEQ ID NO: 128 - protein, 116 aa, synthetic construct
  REGION 1..116: note = Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 129 - protein, 107 aa, synthetic construct
  REGION 1..107: note = Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 130 - protein, 13993 aa, Homo sapiens
TCCTGGCATCAGTTACTGTGTTGACTCACTCAGTGTTGGGATCACTCACTTTCCCCCTACAGGACTCAGATCTGGGAGGCAATTACCTTCGGAGAAAAACGAATAGGAAAAACTGAAGTGTTACTTTTTTTAAAGCTGCTGAAGTTTGTTGGTTTCTCATTGTTTTTAAGCCTACTGGAGCAATAAAGTTTGAAGAACTTTTACCAGGTTTTTTTTATCGCTGCCTTGATATACACTTTTCAAAATGCTTTGGTGGGAAGAAGTAGAGGACTGTTATGAAAGAGAAGATGTTCAAAAGAAAACATTCACAAAATGGGTAAATGCACAATTTTCTAAGTTTGGGAAGCAGCATATTGAGAACCTCTTCAGTGACCTACAGGATGGGAGGCGCCTCCTAGACCTCCTCGAAGGCCTGACAGGGCAAAAACTGCCAAAAGAAAAAGGATCCACAAGAGTTCATGCCCTGAACAATGTCAACAAGGCACTGCGGGTTTTGCAGAACAATAATGTTGATTTAGTGAATATTGGAAGTACTGACATCGTAGATGGAAATCATAAACTGACTCTTGGTTTGATTTGGAATATAATCCTCCACTGGCAGGTCAAAAATGTAATGAAAAATATCATGGCTGGATTGCAACAAACCAACAGTGAAAAGATTCTCCTGAGCTGGGTCCGACAATCAACTCGTAATTATCCACAGGTTAATGTAATCAACTTCACCACCAGCTGGTCTGATGGCCTGGCTTTGAATGCTCTCATCCATAGTCATAGGCCAGACCTATTTGACTGGAATAGTGTGGTTTGCCAGCAGTCAGCCACACAACGACTGGAACATGCATTCAACATCGCCAGATATCAATTAGGCATAGAGAAACTACTCGATCCTGAAGATGTTGATACCACCTATCCAGATAAGAAGTCCATCTTAATGTACATCACATCACTCTTCCAAGTTTTGCCTCAACAAGTGAGCATTGAAGCCATCCAGGAAGTGGAAATGTTGCCAAGGCCACCTAAAGTGACTAAAGAAGAACATTTTCAGTTACATCATCAAATGCACTATTCTCAACAGATCACGGTCAGTCTAGCACAGGGATATGAGAGAACTTCTTCCCCTAAGCCTCGATTCAAGAGCTATGCCTACACACAGGCTGCTTATGTCACCACCTCTGACCCTACACGGAGCCCATTTCCTTCACAGCATTTGGAAGCTCCTGAAGACAAGTCATTTGGCAGTTCATTGATGGAGAGTGAAGTAAACCTGGACCGTTATCAAACAGCTTTAGAAGAAGTATTATCGTGGCTTCTTTCTGCTGAGGACACATTGCAAGCACAAGGAGAGATTTCTAATGATGTGGAAGTGGTGAAAGACCAGTTTCATACTCATGAGGGGTACATGATGGATTTGACAGCCCATCAGGGCCGGGTTGGTAATATTCTACAATTGGGAAGTAAGCTGATTGGAACAGGAAAATTATCAGAAGATGAAGAAACTGAAGTACAAGAGCAGATGAATCTCCTAAATTCAAGATGGGAATGCCTCAGGGTAGCTAGCATGGAAAAACAAAGCAATTTACATAGAGTTTTAATGGATCTCCAGAATCAGAAACTGAAAGAGTTGAATGACTGGCTAACAAAAACAGAAGAAAGAACAAGGAAAATGGAGGAAGAGCCTCTTGGACCTGATCTTGAAGACCTAAAACGCCAAGTACAACAACATAAGGTGCTTCAAGAAGATCTAGAACAAGAACAAGTCAGGGTCAATTCTCTCACTCACATGGTGGTGGTAGTTGATGAATCTAGTGGAGATCACGCAACTGCTGCTTTGGAAGAACAACTTAAGGTATTGGGAGATCGATGGGCAAACATCTGTAGATGGACAGAAGACCGCTGGGTTCTTTTACAAGACATCCTTCTCAAATGGCAACGTCTTACTGAAGAACAGTGCCTTTTTAGTGCATGGCTTTCAGAAAAAGAAGATGCAGTGAACAAGATTCACACAACTGGCTTTAAAGATCAAAATGAAATGTTATCAAGTCTTCAAAAACTGGCCGTTTTAAAAGCGGATCTAGAAAAGAAAAAGCAATCCATGGGCAAACTGTATTCACTCAAACAAGATCTTCTTTCAACACTGAAGAATAAGTCAGTGACCCAGAAGACGGAAGCATGGCTGGATAACTTTGCCCGGTGTTGGGATAATTTAGTCCAAAAACTTGAAAAGAGTACAGCACAGATTTCACAGGCTGTCACCACCACTCAGCCATCACTAACACAGACAACTGTAATGGAAACAGTAACTACGGTGACCACAAGGGAACAGATCCTGGTAAAGCATGCTCAAGAGGAACTTCCACCACCACCTCCCCAAAAGAAGAGGCAGATTACTGTGGATTCTGAAATTAGGAAAAGGTTGGATGTTGATATAACTGAACTTCACAGCTGGATTACTCGCTCAGAAGCTGTGTTGCAGAGTCCTGAATTTGCAATCTTTCGGAAGGAAGGCAACTTCTCAGACTTAAAAGAAAAAGTCAATGCCATAGAGCGAGAAAAAGCTGAGAAGTTCAGAAAACTGCAAGATGCCAGCAGATCAGCTCAGGCCCTGGTGGAACAGATGGTGAATGAGGGTGTTAATGCAGATAGCATCAAACAAGCCTCAGAACAACTGAACAGCCGGTGGATCGAATTCTGCCAGTTGCTAAGTGAGAGACTTAACTGGCTGGAGTATCAGAACAACATCATCGCTTTCTATAATCAGCTACAACAATTGGAGCAGATGACAACTACTGCTGAAAACTGGTTGAAAATCCAACCCACCACCCCATCAGAGCCAACAGCAATTAAAAGTCAGTTAAAAATTTGTAAGGATGAAGTCAACCGGCTATCAGGTCTTCAACCTCAAATTGAACGATTAAAAATTCAAAGCATAGCCCTGAAAGAGAAAGGACAAGGACCCATGTTCCTGGATGCAGACTTTGTGGCCTTTACAAATCATTTTAAGCAAGTCTTTTCTGATGTGCAGGCCAGAGAGAAAGAGCTACAGACAATTTTTGACACTTTGCCACCAATGCGCTATCAGGAGACCATGAGTGCCATCAGGACATGGGTCCAGCAGTCAGAAACCAAACTCTCCATACCTCAACTTAGTGTCACCGACTATGAAATCATGGAGCAGAGACTCGGGGAATTGCAGGCTTTACAAAGTTCTCTGCAAGAGCAACAAAGTGGCCTATACTATCTCAGCACCACTGTGAAAGAGATGTCGAAGAAAGCGCCCTCTGAAATTAGCCGGAAATATCAATCAGAATTTGAAGAAATTGAGGGACGCTGGAAGAAGCTCTCCTCCCAGCTGGTTGAGCATTGTCAAAAGCTAGAGGAGCAAATGAATAAACTCCGAAAAATTCAGAATCACATACAAACCCTGAAGAAATGGATGGCTGAAGTTGATGTTTTTCTGAAGGAGGAATGGCCTGCCCTTGGGGATTCAGAAATTCTAAAAAAGCAGCTGAAACAGTGCAGACTTTTAGTCAGTGATATTCAGACAATTCAGCCCAGTCTAAACAGTGTCAATGAAGGTGGGCAGAAGATAAAGAATGAAGCAGAGCCAGAGTTTGCTTCGAGACTTGAGACAGAACTCAAAGAACTTAACACTCAGTGGGATCACATGTGCCAACAGGTCTATGCCAGAAAGGAGGCCTTGAAGGGAGGTTTGGAGAAAACTGTAAGCCTCCAGAAAGATCTATCAGAGATGCACGAATGGATGACACAAGCTGAAGAAGAGTATCTTGAGAGAGATTTTGAATATAAAACTCCAGATGAATTACAGAAAGCAGTTGAAGAGATGAAGAGAGCTAAAGAAGAGGCCCAACAAAAAGAAGCGAAAGTGAAACTCCTTACTGAGTCTGTAAATAGTGTCATAGCTCAAGCTCCACCTGTAGCACAAGAGGCCTTAAAAAAGGAACTTGAAACTCTAACCACCAACTACCAGTGGCTCTGCACTAGGCTGAATGGGAAATGCAAGACTTTGGAAGAAGTTTGGGCATGTTGGCATGAGTTATTGTCATACTTGGAGAAAGCAAACAAGTGGCTAAATGAAGTAGAATTTAAACTTAAAACCACTGAAAACATTCCTGGCGGAGCTGAGGAAATCTCTGAGGTGCTAGATTCACTTGAAAATTTGATGCGACATTCAGAGGATAACCCAAATCAGATTCGCATATTGGCACAGACCCTAACAGATGGCGGAGTCATGGATGAGCTAATCAATGAGGAACTTGAGACATTTAATTCTCGTTGGAGGGAACTACATGAAGAGGCTGTAAGGAGGCAAAAGTTGCTTGAACAGAGCATCCAGTCTGCCCAGGAGACTGAAAAATCCTTACACTTAATCCAGGAGTCCCTCACATTCATTGACAAGCAGTTGGCAGCTTATATTGCAGACAAGGTGGACGCAGCTCAAATGCCTCAGGAAGCCCAGAAAATCCAATCTGATTTGACAAGTCATGAGATCAGTTTAGAAGAAATGAAGAAACATAATCAGGGGAAGGAGGCTGCCCAAAGAGTCCTGTCTCAGATTGATGTTGCACAGAAAAAATTACAAGATGTCTCCATGAAGTTTCGATTATTCCAGAAACCAGCCAATTTTGAGCAGCGTCTACAAGAAAGTAAGATGATTTTAGATGAAGTGAAGATGCACTTGCCTGCATTGGAAACAAAGAGTGTGGAACAGGAAGTAGTACAGTCACAGCTAAATCATTGTGTGAACTTGTATAAAAGTCTGAGTGAAGTGAAGTCTGAAGTGGAAATGGTGATAAAGACTGGACGTCAGATTGTACAGAAAAAGCAGACGGAAAATCCCAAAGAACTTGATGAAAGAGTAACAGCTTTGAAATTGCATTATAATGAGCTGGGAGCAAAGGTAACAGAAAGAAAGCAACAGTTGGAGAAATGCTTGAAATTGTCCCGTAAGATGCGAAAGGAAATGAATGTCTTGACAGAATGGCTGGCAGCTACAGATATGGAATTGACAAAGAGATCAGCAGTTGAAGGAATGCCTAGTAATTTGGATTCTGAAGTTGCCTGGGGAAAGGCTACTCAAAAAGAGATTGAGAAACAGAAGGTGCACCTGAAGAGTATCACAGAGGTAGGAGAGGCCTTGAAAACAGTTTTGGGCAAGAAGGAGACGTTGGTGGAAGATAAACTCAGTCTTCTGAATAGTAACTGGATAGCTGTCACCTCCCGAGCAGAAGAGTGGTTAAATCTTTTGTTGGAATACCAGAAACACATGGAAACTTTTGACCAGAATGTGGACCACATCACAAAGTGGATCATTCAGGCTGACACACTTTTGGATGAATCAGAGAAAAAGAAACCCCAGCAAAAAGAAGACGTGCTTAAGCGTTTAAAGGCAGAACTGAATGACATACGCCCAAAGGTGGACTCTACACGTGACCAAGCAGCAAACTTGATGGCAAACCGCGGTGACCACTGCAGGAAATTAGTAGAGCCCCAAATCTCAGAGCTCAACCATCGATTTGCAGCCATTTCACACAGAATTAAGACTGGAAAGGCCTCCATTCCTTTGAAGGAATTGGAGCAGTTTAACTCAGATATACAAAAATTGCTTGAACCACTGGAGGCTGAAATTCAGCAGGGGGTGAATCTGAAAGAGGAAGACTTCAATAAAGATATGAATGAAGACAATGAGGGTACTGTAAAAGAATTGTTGCAAAGAGGAGACAACTTACAACAAAGAATCACAGATGAGAGAAAGCGAGAGGAAATAAAGATAAAACAGCAGCTGTTACAGACAAAACATAATGCTCTCAAGGATTTGAGGTCTCAAAGAAGAAAAAAGGCTCTAGAAATTTCTCATCAGTGGTATCAGTACAAGAGGCAGGCTGATGATCTCCTGAAATGCTTGGATGACATTGAAAAAAAATTAGCCAGCCTACCTGAGCCCAGAGATGAAAGGAAAATAAAGGAAATTGATCGGGAATTGCAGAAGAAGAAAGAGGAGCTGAATGCAGTGCGTAGGCAAGCTGAGGGCTTGTCTGAGGATGGGGCCGCAATGGCAGTGGAGCCAACTCAGATCCAGCTCAGCAAGCGCTGGCGGGAAATTGAGAGCAAATTTGCTCAGTTTCGAAGACTCAACTTTGCACAAATTCACACTGTCCGTGAAGAAACGATGATGGTGATGACTGAAGACATGCCTTTGGAAATTTCTTATGTGCCTTCTACTTATTTGACTGAAATCACTCATGTCTCACAAGCCCTATTAGAAGTGGAACAACTTCTCAATGCTCCTGACCTCTGTGCTAAGGACTTTGAAGATCTCTTTAAGCAAGAGGAGTCTCTGAAGAATATAAAAGATAGTCTACAACAAAGCTCAGGTCGGATTGACATTATTCATAGCAAGAAGACAGCAGCATTGCAAAGTGCAACGCCTGTGGAAAGGGTGAAGCTACAGGAAGCTCTCTCCCAGCTTGATTTCCAATGGGAAAAAGTTAACAAAATGTACAAGGACCGACAAGGGCGATTTGACAGATCTGTTGAGAAATGGCGGCGTTTTCATTATGATATAAAGATATTTAATCAGTGGCTAACAGAAGCTGAACAGTTTCTCAGAAAGACACAAATTCCTGAGAATTGGGAACATGCTAAATACAAATGGTATCTTAAGGAACTCCAGGATGGCATTGGGCAGCGGCAAACTGTTGTCAGAACATTGAATGCAACTGGGGAAGAAATAATTCAGCAATCCTCAAAAACAGATGCCAGTATTCTACAGGAAAAATTGGGAAGCCTGAATCTGCGGTGGCAGGAGGTCTGCAAACAGCTGTCAGACAGAAAAAAGAGGCTAGAAGAACAAAAGAATATCTTGTCAGAATTTCAAAGAGATTTAAATGAATTTGTTTTATGGTTGGAGGAAGCAGATAACATTGCTAGTATCCCACTTGAACCTGGAAAAGAGCAGCAACTAAAAGAAAAGCTTGAGCAAGTCAAGTTACTGGTGGAAGAGTTGCCCCTGCGCCAGGGAATTCTCAAACAATTAAATGAAACTGGAGGACCCGTGCTTGTAAGTGCTCCCATAAGCCCAGAAGAGCAAGATAAACTTGAAAATAAGCTCAAGCAGACAAATCTCCAGTGGATAAAGGTTTCCAGAGCTTTACCTGAGAAACAAGGAGAAATTGAAGCTCAAATAAAAGACCTTGGGCAGCTTGAAAAAAAGCTTGAAGACCTTGAAGAGCAGTTAAATCATCTGCTGCTGTGGTTATCTCCTATTAGGAATCAGTTGGAAATTTATAACCAACCAAACCAAGAAGGACCATTTGACGTTCAGGAAACTGAAATAGCAGTTCAAGCTAAACAACCGGATGTGGAAGAGATTTTGTCTAAAGGGCAGCATTTGTACAAGGAAAAACCAGCCACTCAGCCAGTGAAGAGGAAGTTAGAAGATCTGAGCTCTGAGTGGAAGGCGGTAAACCGTTTACTTCAAGAGCTGAGGGCAAAGCAGCCTGACCTAGCTCCTGGACTGACCACTATTGGAGCCTCTCCTACTCAGACTGTTACTCTGGTGACACAACCTGTGGTTACTAAGGAAACTGCCATCTCCAAACTAGAAATGCCATCTTCCTTGATGTTGGAGGTACCTGCTCTGGCAGATTTCAACCGGGCTTGGACAGAACTTACCGACTGGCTTTCTCTGCTTGATCAAGTTATAAAATCACAGAGGGTGATGGTGGGTGACCTTGAGGATATCAACGAGATGATCATCAAGCAGAAGGCAACAATGCAGGATTTGGAACAGAGGCGTCCCCAGTTGGAAGAACTCATTACCGCTGCCCAAAATTTGAAAAACAAGACCAGCAATCAAGAGGCTAGAACAATCATTACGGATCGAATTGAAAGAATTCAGAATCAGTGGGATGAAGTACAAGAACACCTTCAGAACCGGAGGCAACAGTTGAATGAAATGTTAAAGGATTCAACACAATGGCTGGAAGCTAAGGAAGAAGCTGAGCAGGTCTTAGGACAGGCCAGAGCCAAGCTTGAGTCATGGAAGGAGGGTCCCTATACAGTAGATGCAATCCAAAAGAAAATCACAGAAACCAAGCAGTTGGCCAAAGACCTCCGCCAGTGGCAGACAAATGTAGATGTGGCAAATGACTTGGCCCTGAAACTTCTCCGGGATTATTCTGCAGATGATACCAGAAAAGTCCACATGATAACAGAGAATATCAATGCCTCTTGGAGAAGCATTCATAAAAGGGTGAGTGAGCGAGAGGCTGCTTTGGAAGAAACTCATAGATTACTGCAACAGTTCCCCCTGGACCTGGAAAAGTTTCTTGCCTGGCTTACAGAAGCTGAAACAACTGCCAATGTCCTACAGGATGCTACCCGTAAGGAAAGGCTCCTAGAAGACTCCAAGGGAGTAAAAGAGCTGATGAAACAATGGCAAGACCTCCAAGGTGAAATTGAAGCTCACACAGATGTTTATCACAACCTGGATGAAAACAGCCAAAAAATCCTGAGATCCCTGGAAGGTTCCGATGATGCAGTCCTGTTACAAAGACGTTTGGATAACATGAACTTCAAGTGGAGTGAACTTCGGAAAAAGTCTCTCAACATTAGGTCCCATTTGGAAGCCAGTTCTGACCAGTGGAAGCGTCTGCACCTTTCTCTGCAGGAACTTCTGGTGTGGCTACAGCTGAAAGATGATGAATTAAGCCGGCAGGCACCTATTGGAGGCGACTTTCCAGCAGTTCAGAAGCAGAACGATGTACATAGGGCCTTCAAGAGGGAATTGAAAACTAAAGAACCTGTAATCATGAGTACTCTTGAGACTGTACGAATATTTCTGACAGAGCAGCCTTTGGAAGGACTAGAGAAACTCTACCAGGAGCCCAGAGAGCTGCCTCCTGAGGAGAGAGCCCAGAATGTCACTCGGCTTCTACGAAAGCAGGCTGAGGAGGTCAATACTGAGTGGGAAAAATTGAACCTGCACTCCGCTGACTGGCAGAGAAAAATAGATGAGACCCTTGAAAGACTCCAGGAACTTCAAGAGGCCACGGATGAGCTGGACCTCAAGCTGCGCCAAGCTGAGGTGATCAAGGGATCCTGGCAGCCCGTGGGCGATCTCCTCATTGACTCTCTCCAAGATCACCTCGAGAAAGTCAAGGCACTTCGAGGAGAAATTGCGCCTCTGAAAGAGAACGTGAGCCACGTCAATGACCTTGCTCGCCAGCTTACCACTTTGGGCATTCAGCTCTCACCGTATAACCTCAGCACTCTGGAAGACCTGAACACCAGATGGAAGCTTCTGCAGGTGGCCGTCGAGGACCGAGTCAGGCAGCTGCATGAAGCCCACAGGGACTTTGGTCCAGCATCTCAGCACTTTCTTTCCACGTCTGTCCAGGGTCCCTGGGAGAGAGCCATCTCGCCAAACAAAGTGCCCTACTATATCAACCACGAGACTCAAACAACTTGCTGGGACCATCCCAAAATGACAGAGCTCTACCAGTCTTTAGCTGACCTGAATAATGTCAGATTCTCAGCTTATAGGACTGCCATGAAACTCCGAAGACTGCAGAAGGCCCTTTGCTTGGATCTCTTGAGCCTGTCAGCTGCATGTGATGCCTTGGACCAGCACAACCTCAAGCAAAATGACCAGCCCATGGATATCCTGCAGATTATTAATTGTTTGACCACTATTTATGACCGCCTGGAGCAAGAGCACAACAATTTGGTCAACGTCCCTCTCTGCGTGGATATGTGTCTGAACTGGCTGCTGAATGTTTATGATACGGGACGAACAGGGAGGATCCGTGTCCTGTCTTTTAAAACTGGCATCATTTCCCTGTGTAAAGCACATTTGGAAGACAAGTACAGATACCTTTTCAAGCAAGTGGCAAGTTCAACAGGATTTTGTGACCAGCGCAGGCTGGGCCTCCTTCTGCATGATTCTATCCAAATTCCAAGACAGTTGGGTGAAGTTGCATCCTTTGGGGGCAGTAACATTGAGCCAAGTGTCCGGAGCTGCTTCCAATTTGCTAATAATAAGCCAGAGATCGAAGCGGCCCTCTTCCTAGACTGGATGAGACTGGAACCCCAGTCCATGGTGTGGCTGCCCGTCCTGCACAGAGTGGCTGCTGCAGAAACTGCCAAGCATCAGGCCAAATGTAACATCTGCAAAGAGTGTCCAATCATTGGATTCAGGTACAGGAGTCTAAAGCACTTTAATTATGACATCTGCCAAAGCTGCTTTTTTTCTGGTCGAGTTGCAAAAGGCCATAAAATGCACTATCCCATGGTGGAATATTGCACTCCGACTACATCAGGAGAAGATGTTCGAGACTTTGCCAAGGTACTAAAAAACAAATTTCGAACCAAAAGGTATTTTGCGAAGCATCCCCGAATGGGCTACCTGCCAGTGCAGACTGTCTTAGAGGGGGACAACATGGAAACTCCCGTTACTCTGATCAACTTCTGGCCAGTAGATTCTGCGCCTGCCTCGTCCCCTCAGCTTTCACACGATGATACTCATTCACGCATTGAACATTATGCTAGCAGGCTAGCAGAAATGGAAAACAGCAATGGATCTTATCTAAATGATAGCATCTCTCCTAATGAGAGCATAGATGATGAACATTTGTTAATCCAGCATTACTGCCAAAGTTTGAACCAGGACTCCCCCCTGAGCCAGCCTCGTAGTCCTGCCCAGATCTTGATTTCCTTAGAGAGTGAGGAAAGAGGGGAGCTAGAGAGAATCCTAGCAGATCTTGAGGAAGAAAACAGGAATCTGCAAGCAGAATATGACCGTCTAAAGCAGCAGCACGAACATAAAGGCCTGTCCCCACTGCCGTCCCCTCCTGAAATGATGCCCACCTCTCCCCAGAGTCCCCGGGATGCTGAGCTCATTGCTGAGGCCAAGCTACTGCGTCAACACAAAGGCCGCCTGGAAGCCAGGATGCAAATCCTGGAAGACCACAATAAACAGCTGGAGTCACAGTTACACAGGCTAAGGCAGCTGCTGGAGCAACCCCAGGCAGAGGCCAAAGTGAATGGCACAACGGTGTCCTCTCCTTCTACCTCTCTACAGAGGTCCGACAGCAGTCAGCCTATGCTGCTCCGAGTGGTTGGCAGTCAAACTTCGGACTCCATGGGTGAGGAAGATCTTCTCAGTCCTCCCCAGGACACAAGCACAGGGTTAGAGGAGGTGATGGAGCAACTCAACAACTCCTTCCCTAGTTCAAGAGGAAGAAATACCCCTGGAAAGCCAATGAGAGAGGACACAATGTAGGAAGTCTTTTCCACATGGCAGATGATTTGGGCAGAGCGATGGAGTCCTTAGTATCAGTCATGACAGATGAAGAAGGAGCAGAATAAATGTTTTACAACTCCTGATTCCCGCATGGTTTTTATAATATTCATACAACAAAGAGGATTAGACAGTAAGAGTTTACAAGAAATAAATCTATATTTTTGTGAAGGGTAGTGGTATTATACTGTAGATTTCAGTAGTTTCTAAGTCTGTTATTGTTTTGTTAACAATGGCAGGTTTTACACGTCTATGCAATTGTACAAAAAAGTTATAAGAAAACTACATGTAAAATCTTGATAGCTAAATAACTTGCCATTTCTTTATATGGAACGCATTTTGGGTTGTTTAAAAATTTATAACAGTTATAAAGAAAGATTGTAAACTAAAGTGTGCTTTATAAAAAAAAGTTGTTTATAAAAACCCCTAAAAACAAAACAAACACACACACACACACATACACACACACACACAAAACTTTGAGGCAGCGCATTGTTTTGCATCCTTTTGGCGTGATATCCATATGAAATTCATGGCTTTTTCTTTTTTTGCATATTAAAGATAAGACTTCCTCTACCACCACACCAAATGACTACTACACACTGCTCATTTGAGAACTGTCAGCTGAGTGGGGCAGGCTTGAGTTTTCATTTCATATATCTATATGTCTATAAGTATATAAATACTATAGTTATATAGATAAAGAGATACGAATTTCTATAGACTGACTTTTTCCATTTTTTAAATGTTCATGTCACATCCTAATAGAAAGAAATTACTTCTAGTCAGTCATCCAGGCTTACCTGCTTGGTCTAGAATGGATTTTTCCCGGAGCCGGAAGCCAGGAGGAAACTACACCACACTAAAACATTGTCTACAGCTCCAGATGTTTCTCATTTTAAACAACTTTCCACTGACAACGAAAGTAAAGTAAAGTATTGGATTTTTTTAAAGGGAACATGTGAATGAATACACAGGACTTATTATATCAGAGTGAGTAATCGGTTGGTTGGTTGATTGATTGATTGATTGATACATTCAGCTTCCTGCTGCTAGCAATGCCACGATTTAGATTTAATGATGCTTCAGTGGAAATCAATCAGAAGGTATTCTGACCTTGTGAACATCAGAAGGTATTTTTTAACTCCCAAGCAGTAGCAGGACGATGATAGGGCTGGAGGGCTATGGATTCCCAGCCCATCCCTGTGAAGGAGTAGGCCACTCTTTAAGTGAAGGATTGGATGATTGTTCATAATACATAAAGTTCTCTGTAATTACAACTAAATTATTATGCCCTCTTCTCACAGTCAAAAGGAACTGGGTGGTTTGGTTTTTGTTGCTTTTTTAGATTTATTGTCCCATGTGGGATGAGTTTTTAAATGCCACAAGACATAATTTAAAATAAATAAACTTTGGGAAAAGGTGTAAAACAGTAGCCCCATCACATTTGTGATACTGACAGGTATCAACCCAGAAGCCCATGAACTGTGTTTCCATCCTTTGCATTTCTCTGCGAGTAGTTCCACACAGGTTTGTAAGTAAGTAAGAAAGAAGGCAAATTGATTCAAATGTTACAAAAAAACCCTTCTTGGTGGATTAGACAGGTTAAATATATAAACAAACAAACAAAAATTGCTCAAAAAAGAGGAGAAAAGCTCAAGAGGAAAAGCTAAGGACTGGTAGGAAAAAGCTTTACTCTTTCATGCCATTTTATTTCTTTTTGATTTTTAAATCATTCATTCAATAGATACCACCGTGTGACCTATAATTTTGCAAATCTGTTACCTCTGACATCAAGTGTAATTAGCTTTTGGAGAGTGGGCTGACATCAAGTGTAATTAGCTTTTGGAGAGTGGGTTTTGTCCATTATTAATAATTAATTAATTAACATCAAACACGGCTTCTCATGCTATTTCTACCTCACTTTGGTTTTGGGGTGTTCCTGATAATTGTGCACACCTGAGTTCACAGCTTCACCACTTGTCCATTGCGTTATTTTCTTTTTCCTTTATAATTCTTTCTTTTTCCTTCATAATTTTCAAAAGAAAACCCAAAGCTCTAAGGTAACAAATTACCAAATTACATGAAGATTTGGTTTTTGTCTTGCATTTTTTTCCTTTATGTGACGCTGGACCTTTTCTTTACCCAAGGATTTTTAAAACTCAGATTTAAAACAAGGGGTTACTTTACATCCTACTAAGAAGTTTAAGTAAGTAAGTTTCATTCTAAAATCAGAGGTAAATAGAGTGCATAAATAATTTTGTTTTAATCTTTTTGTTTTTCTTTTAGACACATTAGCTCTGGAGTGAGTCTGTCATAATATTTGAACAAAAATTGAGAGCTTTATTGCTGCATTTTAAGCATAATTAATTTGGACATTATTTCGTGTTGTGTTCTTTATAACCACCAAGTATTAAACTGTAAATCATAATGTAACTGAAGCATAAACATCACATGGCATGTTTTGTCATTGTTTTCAGGTACTGAGTTCTTACTTGAGTATCATAATATATTGTGTTTTAACACCAACACTGTAACATTTACGAATTATTTTTTTAAACTTCAGTTTTACTGCATTTTCACAACATATCAGACTTCACCAAATATATGCCTTACTATTGTATTATAGTACTGCTTTACTGTGTATCTCAATAAAGCACGCAGTTATGTTAC

SEQ ID NO: 131 - protein, 173 aa, Homo sapiens
AATATAAAAGATAGTCTACAACAAAGCTCAGGTCGGATTGACATTATTCATAGCAAGAAGACAGCAGCATTGCAAAGTGCAACGCCTGTGGAAAGGGTGAAGCTACAGGAAGCTCTCTCCCAGCTTGATTTCCAATGGGAAAAAGTTAACAAAATGTACAAGGACCGACAAGG

SEQ ID NO: 132 - protein, 446 aa, synthetic construct
  REGION 1..446: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 214 aa, synthetic construct
  REGION 1..214: note = Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 446 aa, Homo sapiens
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 214 aa, Homo sapiens
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 226 aa, synthetic construct
  REGION 1..226: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 137 - protein, 226 aa, Homo sapiens
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 138 - protein, 8 aa, synthetic construct
  REGION 1..8: note = Synthetic
GYSFTSYW

SEQ ID NO: 139 - protein, 8 aa, synthetic construct
  REGION 1..8: note = Synthetic
IYPGDSDT

SEQ ID NO: 140 - protein, 15 aa, synthetic construct
  REGION 1..15: note = Synthetic
ARFPYDSSGYYSFDY

SEQ ID NO: 141 - protein, 6 aa, synthetic construct
  REGION 1..6: note = Synthetic
QSISSY

SEQ ID NO: 142 - skipped

SEQ ID NO: 143 - protein, 9 aa, synthetic construct
  REGION 1..9: note = Synthetic
QQSYSTPLT

SEQ ID NO: 144 - protein, 5 aa, synthetic construct
  REGION 1..5: note = Synthetic
SYWIG

SEQ ID NO: 145 - protein, 18 aa, synthetic construct
  REGION 1..18: note = Synthetic
IIYPGDSDTRYSPSFQGQ

SEQ ID NO: 146 - protein, 13 aa, synthetic construct
  REGION 1..13: note = Synthetic
FPYDSSGYYSFDY

SEQ ID NO: 147 - protein, 11 aa, synthetic construct
  REGION 1..11: note = Synthetic
RASQSISSYLN

SEQ ID NO: 148 - protein, 7 aa, synthetic construct
  REGION 1..7: note = Synthetic
AASSLQS

SEQ ID NO: 149 - protein, 7 aa, synthetic construct
  REGION 1..7: note = Synthetic
GYSFTSY

SEQ ID NO: 150 - skipped

SEQ ID NO: 151 - protein, 11 aa, synthetic construct
  REGION 1..11: note = Synthetic
PYDSSGYYSFD

SEQ ID NO: 152 - protein, 7 aa, synthetic construct
  REGION 1..7: note = Synthetic
SQSISSY

SEQ ID NO: 153 - protein, 6 aa, synthetic construct
  REGION 1..6: note = Synthetic
SYSTPL

SEQ ID NO: 154 - protein, 122 aa, synthetic construct
  REGION 1..122: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 155 - protein, 107 aa, synthetic construct [sequence omitted]

SEQ ID NO: 156 - protein, 452 aa, synthetic construct
  REGION 1..452: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 232 aa, synthetic construct
  REGION 1..232: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 159 - protein, 230 aa, synthetic construct
  REGION 1..230: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 160 - RNA, 23 nt, synthetic construct
  misc_feature 1..23: note = Synthetic
  source 1..23: mol_type = other RNA
cagatctgttgagaaatggcggc

SEQ ID NO: 161 - RNA, 24 nt, synthetic construct
  misc_feature 1..24: note = Synthetic
  source 1..24: mol_type = other RNA
cagatctgttgagaaatggcggcg

SEQ ID NO: 162 - RNA, 25 nt, synthetic construct
  misc_feature 1..25: note = Synthetic
  source 1..25: mol_type = other RNA
cagatctgttgagaaatggcggcgt

SEQ ID NO: 163 - RNA, 22 nt, synthetic construct
  misc_feature 1..22: note = Synthetic
  source 1..22: mol_type = other RNA
agatctgttgagaaatggcggc

SEQ ID NO: 164 - RNA, 23 nt, synthetic construct
  misc_feature 1..23: note = Synthetic
  source 1..23: mol_type = other RNA
agatctgttgagaaatggcggcg

SEQ ID NO: 165 - RNA, 24 nt, synthetic construct
  misc_feature 1..24: note = Synthetic
  source 1..24: mol_type = other RNA
agatctgttgagaaatggcggcgt

SEQ ID NO: 166 - RNA, 25 nt, synthetic construct
  misc_feature 1..25: note = Synthetic
  source 1..25: mol_type = other RNA
agatctgttgagaaatggcggcgtt

SEQ ID NO: 167 - RNA, 21 nt, synthetic construct
  misc_feature 1..21: note = Synthetic
  source 1..21: mol_type = other RNA
gatctgttgagaaatggcggc

SEQ ID NO: 168 - RNA, 22 nt, synthetic construct
  misc_feature 1..22: note = Synthetic
  source 1..22: mol_type = other RNA
gatctgttgagaaatggcggcg

SEQ ID NO: 169 - RNA, 23 nt, synthetic construct
  misc_feature 1..23: note = Synthetic
  source 1..23: mol_type = other RNA
gatctgttgagaaatggcggcgt

SEQ ID NO: 170 - RNA, 24 nt, synthetic construct
  misc_feature 1..24: note = Synthetic
  source 1..24: mol_type = other RNA
gatctgttgagaaatggcggcgtt

SEQ ID NO: 171 - RNA, 25 nt, synthetic construct
  misc_feature 1..25: note = Synthetic
  source 1..25: mol_type = other RNA
gatctgttgagaaatggcggcgttt

SEQ ID NO: 172 - RNA, 25 nt, synthetic construct
  misc_feature 1..25: note = Synthetic
  source 1..25: mol_type = other RNA
tctgttgagaaatggcggcgttttc

SEQ ID NO: 173 - RNA, 20 nt, synthetic construct
  misc_feature 1..20: note = Synthetic
  source 1..20: mol_type = other RNA
ctgttgagaaatggcggcgt

SEQ ID NO: 174 - RNA, 25 nt, synthetic construct
  misc_feature 1..25: note = Synthetic
  source 1..25: mol_type = other RNA
ctgttgagaaatggcggcgttttca

SEQ ID NO: 175 - RNA, 25 nt, synthetic construct
  misc_feature 1..25: note = Synthetic
  source 1..25: mol_type = other RNA
tgagaaatggcggcgttttcattat

SEQ ID NO: 176 - RNA, 25 nt, synthetic construct
  misc_feature 1..25: note = Synthetic
  source 1..25: mol_type = other RNA
gagaaatggcggcgttttcattatg

SEQ ID NO: 177 - RNA, 25 nt, synthetic construct
  misc_feature 1..25: note = Synthetic
  source 1..25: mol_type = other RNA
agaaatggcggcgttttcattatga

SEQ ID NO: 178 - RNA, 25 nt, synthetic construct
  misc_feature 1..25: note = Synthetic
  source 1..25: mol_type = other RNA
aatcagtggctaacagaagctgaac

SEQ ID NO: 179 - RNA, 24 nt, synthetic construct
  misc_feature 1..24: note = Synthetic
  source 1..24: mol_type = other RNA
atcagtggctaacagaagctgaac

SEQ ID NO: 180 - RNA, 25 nt, synthetic construct
  misc_feature 1..25: note = Synthetic
  source 1..25: mol_type = other RNA
atcagtggctaacagaagctgaaca

SEQ ID NO: 181 - RNA, 23 nt, synthetic construct
  misc_feature 1..23: note = Synthetic
  source 1..23: mol_type = other RNA
tcagtggctaacagaagctgaac

SEQ ID NO: 182 - RNA, 24 nt, synthetic construct
  misc_feature 1..24: note = Synthetic
  source 1..24: mol_type = other RNA
tcagtggctaacagaagctgaaca

SEQ ID NO: 183 - RNA, 25 nt, synthetic construct
  misc_feature 1..25: note = Synthetic
  source 1..25: mol_type = other RNA
tcagtggctaacagaagctgaacag

SEQ ID NO: 184 - RNA, 22 nt, synthetic construct
  misc_feature 1..22: note = Synthetic
  source 1..22: mol_type = other RNA
cagtggctaacagaagctgaac

SEQ ID NO: 185 - RNA, 23 nt, synthetic construct
  misc_feature 1..23: note = Synthetic
  source 1..23: mol_type = other RNA
cagtggctaacagaagctgaaca

SEQ ID NO: 186 - RNA, 24 nt, synthetic construct
  misc_feature 1..24: note = Synthetic
  source 1..24: mol_type = other RNA
cagtggctaacagaagctgaacag

SEQ ID NO: 187 - RNA, 25 nt, synthetic construct
  misc_feature 1..25: note = Synthetic
  source 1..25: mol_type = other RNA
cagtggctaacagaagctgaacagt

SEQ ID NO: 188 - RNA, 23 nt, synthetic construct
  misc_feature 1..23: note = Synthetic
  source 1..23: mol_type = other RNA
agtggctaacagaagctgaacag

SEQ ID NO: 189 - RNA, 24 nt, synthetic construct
  misc_feature 1..24: note = Synthetic
  source 1..24: mol_type = other RNA
agtggctaacagaagctgaacagt

SEQ ID NO: 190 - RNA, 25 nt, synthetic construct
  misc_feature 1..25: note = Synthetic
  source 1..25: mol_type = other RNA
agtggctaacagaagctgaacagtt

SEQ ID NO: 191 - RNA, 23 nt, synthetic construct
  misc_feature 1..23: note = Synthetic
  source 1..23: mol_type = other RNA
gtggctaacagaagctgaacagt

SEQ ID NO: 192 - RNA, 24 nt, synthetic construct
  misc_feature 1..24: note = Synthetic
  source 1..24: mol_type = other RNA
gtggctaacagaagctgaacagtt

SEQ ID NO: 193 - RNA, 25 nt, synthetic construct
  misc_feature 1..25: note = Synthetic
  source 1..25: mol_type = other RNA
aaagatcaggttctgaagggtgatg

SEQ ID NO: 194 - RNA, 24 nt, synthetic construct
  misc_feature 1..24: note = Synthetic
  source 1..24: mol_type = other RNA
aagatcaggttctgaagggtgatg

SEQ ID NO: 195 - RNA, 23 nt, synthetic construct
  misc_feature 1..23: note = Synthetic
  source 1..23: mol_type = other RNA
agatcaggttctgaagggtgatg

SEQ ID NO: 196 - RNA, 23 nt, synthetic construct
  misc_feature 1..23: note = Synthetic
  source 1..23: mol_type = other RNA
gccgccatttctcaacagatctg

SEQ ID NO: 197 - RNA, 24 nt, synthetic construct
  misc_feature 1..24: note = Synthetic
  source 1..24: mol_type = other RNA
cgccgccatttctcaacagatctg

SEQ ID NO: 198 - RNA, 25 nt, synthetic construct
  misc_feature 1..25: note = Synthetic
  source 1..25: mol_type = other RNA
acgccgccatttctcaacagatctg

SEQ ID NO: 199 - RNA, 22 nt, synthetic construct
  misc_feature 1..22: note = Synthetic
  source 1..22: mol_type = other RNA
gccgccatttctcaacagatct

SEQ ID NO: 200 - RNA, 23 nt, synthetic construct
  misc_feature 1..23: note = Synthetic
  source 1..23: mol_type = other RNA
cgccgccatttctcaacagatct

SEQ ID NO: 201 - RNA, 24 nt, synthetic construct
  misc_feature 1..24: note = Synthetic
  source 1..24: mol_type = other RNA
acgccgccatttctcaacagatct

SEQ ID NO: 202 - RNA, 25 nt, synthetic construct
  misc_feature 1..25: note = Synthetic
  source 1..25: mol_type = other RNA
aacgccgccatttctcaacagatct

SEQ ID NO: 203 - RNA, 21 nt, synthetic construct
  misc_feature 1..21: note = Synthetic
  source 1..21: mol_type = other RNA
gccgccatttctcaacagatc

SEQ ID NO: 204 - RNA, 22 nt, synthetic construct
  misc_feature 1..22: note = Synthetic
  source 1..22: mol_type = other RNA
cgccgccatttctcaacagatc

SEQ ID NO: 205 - RNA, 23 nt, synthetic construct
  misc_feature 1..23: note = Synthetic
  source 1..23: mol_type = other RNA
acgccgccatttctcaacagatc

SEQ ID NO: 206 - RNA, 24 nt, synthetic construct
  misc_feature 1..24: note = Synthetic
  source 1..24: mol_type = other RNA
aacgccgccatttctcaacagatc

SEQ ID NO: 207 - RNA, 25 nt, synthetic construct
  misc_feature 1..25: note = Synthetic
  source 1..25: mol_type = other RNA
aaacgccgccatttctcaacagatc

SEQ ID NO: 208 - RNA, 25 nt, synthetic construct
  misc_feature 1..25: note = Synthetic
  source 1..25: mol_type = other RNA
gaaaacgccgccatttctcaacaga

SEQ ID NO: 209 - RNA, 20 nt, synthetic construct
  misc_feature 1..20: note = Synthetic
  source 1..20: mol_type = other RNA
acgccgccatttctcaacag

SEQ ID NO: 210 - RNA, 25 nt, synthetic construct
  misc_feature 1..25: note = Synthetic
  source 1..25: mol_type = other RNA
tgaaaacgccgccatttctcaacag

SEQ ID NO: 211 - RNA, 25 nt, synthetic construct
  misc_feature 1..25: note = Synthetic
  source 1..25: mol_type = other RNA
ataatgaaaacgccgccatttctca

SEQ ID NO: 212 - RNA, 25 nt, synthetic construct
  misc_feature 1..25: note = Synthetic
  source 1..25: mol_type = other RNA
cataatgaaaacgccgccatttctc

SEQ ID NO: 213 - RNA, 25 nt, synthetic construct
  misc_feature 1..25: note = Synthetic
  source 1..25: mol_type = other RNA
tcataatgaaaacgccgccatttct

SEQ ID NO: 214 - RNA, 25 nt, synthetic construct
  misc_feature 1..25: note = Synthetic
  source 1..25: mol_type = other RNA
gttcagcttctgttagccactgatt

SEQ ID NO: 215 - RNA, 24 nt, synthetic construct
  misc_feature 1..24: note = Synthetic
  source 1..24: mol_type = other RNA
gttcagcttctgttagccactgat

SEQ ID NO: 216 - RNA, 25 nt, synthetic construct
  misc_feature 1..25: note = Synthetic
  source 1..25: mol_type = other RNA
tgttcagcttctgttagccactgat

SEQ ID NO: 217 - RNA, 23 nt, synthetic construct
  misc_feature 1..23: note = Synthetic
  source 1..23: mol_type = other RNA
gttcagcttctgttagccactga

SEQ ID NO: 218 - RNA, 24 nt, synthetic construct
  misc_feature 1..24: note = Synthetic
  source 1..24: mol_type = other RNA
tgttcagcttctgttagccactga

SEQ ID NO: 219 - RNA, 25 nt, synthetic construct
  misc_feature 1..25: note = Synthetic
  source 1..25: mol_type = other RNA
ctgttcagcttctgttagccactga

SEQ ID NO: 220 - RNA, 22 nt, synthetic construct
  misc_feature 1..22: note = Synthetic
  source 1..22: mol_type = other RNA
gttcagcttctgttagccactg

SEQ ID NO: 221 - RNA, 23 nt, synthetic construct
  misc_feature 1..23: note = Synthetic
  source 1..23: mol_type = other RNA
tgttcagcttctgttagccactg

SEQ ID NO: 222 - RNA, 24 nt, synthetic construct
  misc_feature 1..24: note = Synthetic
  source 1..24: mol_type = other RNA
ctgttcagcttctgttagccactg

SEQ ID NO: 223 - RNA, 25 nt, synthetic construct
  misc_feature 1..25: note = Synthetic
  source 1..25: mol_type = other RNA
actgttcagcttctgttagccactg

SEQ ID NO: 224 - RNA, 23 nt, synthetic construct
  misc_feature 1..23: note = Synthetic
  source 1..23: mol_type = other RNA
ctgttcagcttctgttagccact

SEQ ID NO: 225 - RNA, 24 nt, synthetic construct
  misc_feature 1..24: note = Synthetic
  source 1..24: mol_type = other RNA
actgttcagcttctgttagccact

SEQ ID NO: 226 - RNA, 25 nt, synthetic construct
  misc_feature 1..25: note = Synthetic
  source 1..25: mol_type = other RNA
aactgttcagcttctgttagccact

SEQ ID NO: 227 - RNA, 23 nt, synthetic construct
  misc_feature 1..23: note = Synthetic
  source 1..23: mol_type = other RNA
actgttcagcttctgttagccac

SEQ ID NO: 228 - RNA, 24 nt, synthetic construct
  misc_feature 1..24: note = Synthetic
  source 1..24: mol_type = other RNA
aactgttcagcttctgttagccac

SEQ ID NO: 229 - RNA, 25 nt, synthetic construct
  misc_feature 1..25: note = Synthetic
  source 1..25: mol_type = other RNA
catcacccttcagaacctgatcttt

SEQ ID NO: 230 - RNA, 24 nt, synthetic construct
  misc_feature 1..24: note = Synthetic
  source 1..24: mol_type = other RNA
catcacccttcagaacctgatctt

SEQ ID NO: 231 - RNA, 23 nt, synthetic construct
  misc_feature 1..23: note = Synthetic
  source 1..23: mol_type = other RNA
catcacccttcagaacctgatct

SEQ ID NO: 232 - DNA, 23 bp, synthetic construct
  misc_feature 1..23: note = Synthetic
  source 1..23: mol_type = other DNA
gccgccatttctcaacagatctg

SEQ ID NO: 233 - DNA, 24 bp, synthetic construct
  misc_feature 1..24: note = Synthetic
  source 1..24: mol_type = other DNA
cgccgccatttctcaacagatctg

SEQ ID NO: 234 - DNA, 25 bp, synthetic construct
  misc_feature 1..25: note = Synthetic
  source 1..25: mol_type = other DNA
acgccgccatttctcaacagatctg

SEQ ID NO: 235 - DNA, 22 bp, synthetic construct
  misc_feature 1..22: note = Synthetic
  source 1..22: mol_type = other DNA
gccgccatttctcaacagatct

SEQ ID NO: 236 - DNA, 23 bp, synthetic construct
  misc_feature 1..23: note = Synthetic
  source 1..23: mol_type = other DNA
cgccgccatttctcaacagatct

SEQ ID NO: 237 - DNA, 24 bp, synthetic construct
  misc_feature 1..24: note = Synthetic
  source 1..24: mol_type = other DNA
acgccgccatttctcaacagatct

SEQ ID NO: 238 - DNA, 25 bp, synthetic construct
  misc_feature 1..25: note = Synthetic
  source 1..25: mol_type = other DNA
aacgccgccatttctcaacagatct

SEQ ID NO: 239 - DNA, 21 bp, synthetic construct
  misc_feature 1..21: note = Synthetic
  source 1..21: mol_type = other DNA
gccgccatttctcaacagatc

SEQ ID NO: 240 - DNA, 22 bp, synthetic construct
  misc_feature 1..22: note = Synthetic
  source 1..22: mol_type = other DNA
cgccgccatttctcaacagatc

SEQ ID NO: 241 - DNA, 23 bp, synthetic construct
  misc_feature 1..23: note = Synthetic
  source 1..23: mol_type = other DNA
acgccgccatttctcaacagatc

SEQ ID NO: 242 - DNA, 24 bp, synthetic construct
  misc_feature 1..24: note = Synthetic
  source 1..24: mol_type = other DNA
aacgccgccatttctcaacagatc

SEQ ID NO: 243 - DNA, 25 bp, synthetic construct
  misc_feature 1..25: note = Synthetic
  source 1..25: mol_type = other DNA
aaacgccgccatttctcaacagatc

SEQ ID NO: 244 - DNA, 25 bp, synthetic construct
  misc_feature 1..25: note = Synthetic
  source 1..25: mol_type = other DNA
gaaaacgccgccatttctcaacaga

SEQ ID NO: 245 - DNA, 20 bp, synthetic construct
  misc_feature 1..20: note = Synthetic
  source 1..20: mol_type = other DNA
acgccgccatttctcaacag

SEQ ID NO: 246 - DNA, 25 bp, synthetic construct
  misc_feature 1..25: note = Synthetic
  source 1..25: mol_type = other DNA
tgaaaacgccgccatttctcaacag

SEQ ID NO: 247 - DNA, 25 bp, synthetic construct
  misc_feature 1..25: note = Synthetic
  source 1..25: mol_type = other DNA
ataatgaaaacgccgccatttctca

SEQ ID NO: 248 - DNA, 25 bp, synthetic construct
  misc_feature 1..25: note = Synthetic
  source 1..25: mol_type = other DNA
cataatgaaaacgccgccatttctc

SEQ ID NO: 249 - DNA, 25 bp, synthetic construct
  misc_feature 1..25: note = Synthetic
  source 1..25: mol_type = other DNA
tcataatgaaaacgccgccatttct

SEQ ID NO: 250 - DNA, 25 bp, synthetic construct
  misc_feature 1..25: note = Synthetic
  source 1..25: mol_type = other DNA
gttcagcttctgttagccactgatt

SEQ ID NO: 251 - DNA, 24 bp, synthetic construct
  misc_feature 1..24: note = Synthetic
  source 1..24: mol_type = other DNA
gttcagcttctgttagccactgat

SEQ ID NO: 252 - DNA, 25 bp, synthetic construct
  misc_feature 1..25: note = Synthetic
  source 1..25: mol_type = other DNA
tgttcagcttctgttagccactgat

SEQ ID NO: 253 - DNA, 23 bp, synthetic construct
  misc_feature 1..23: note = Synthetic
  source 1..23: mol_type = other DNA
gttcagcttctgttagccactga

SEQ ID NO: 254 - DNA, 24 bp, synthetic construct
  misc_feature 1..24: note = Synthetic
  source 1..24: mol_type = other DNA
tgttcagcttctgttagccactga

SEQ ID NO: 255 - DNA, 25 bp, synthetic construct
  misc_feature 1..25: note = Synthetic
  source 1..25: mol_type = other DNA
ctgttcagcttctgttagccactga

SEQ ID NO: 256 - DNA, 22 bp, synthetic construct
  misc_feature 1..22: note = Synthetic
  source 1..22: mol_type = other DNA
gttcagcttctgttagccactg

SEQ ID NO: 257 - DNA, 23 bp, synthetic construct
  misc_feature 1..23: note = Synthetic
  source 1..23: mol_type = other DNA
tgttcagcttctgttagccactg

SEQ ID NO: 258 - DNA, 24 bp, synthetic construct
  misc_feature 1..24: note = Synthetic
  source 1..24: mol_type = other DNA
ctgttcagcttctgttagccactg

SEQ ID NO: 259 - DNA, 25 bp, synthetic construct
  misc_feature 1..25: note = Synthetic
  source 1..25: mol_type = other DNA
actgttcagcttctgttagccactg

SEQ ID NO: 260 - DNA, 23 bp, synthetic construct
  misc_feature 1..23: note = Synthetic
  source 1..23: mol_type = other DNA
ctgttcagcttctgttagccact

SEQ ID NO: 261 - DNA, 24 bp, synthetic construct
  misc_feature 1..24: note = Synthetic
  source 1..24: mol_type = other DNA
actgttcagcttctgttagccact

SEQ ID NO: 262 - DNA, 25 bp, synthetic construct
  misc_feature 1..25: note = Synthetic
  source 1..25: mol_type = other DNA
aactgttcagcttctgttagccact

SEQ ID NO: 263 - DNA, 23 bp, synthetic construct
  misc_feature 1..23: note = Synthetic
  source 1..23: mol_type = other DNA
actgttcagcttctgttagccac

SEQ ID NO: 264 - DNA, 24 bp, synthetic construct
  misc_feature 1..24: note = Synthetic
  source 1..24: mol_type = other DNA
aactgttcagcttctgttagccac

SEQ ID NO: 265 - DNA, 25 bp, synthetic construct
  misc_feature 1..25: note = Synthetic
  source 1..25: mol_type = other DNA
catcacccttcagaacctgatcttt

SEQ ID NO: 266 - DNA, 24 bp, synthetic construct
  misc_feature 1..24: note = Synthetic
  source 1..24: mol_type = other DNA
catcacccttcagaacctgatctt

SEQ ID NO: 267 - DNA, 23 bp, synthetic construct
  misc_feature 1..23: note = Synthetic
  source 1..23: mol_type = other DNA
catcacccttcagaacctgatct

SEQ ID NO: 268 - DNA, 60 bp, Homo sapiens
  source 1..60: mol_type = genomic DNA
agttaacaaaatgtacaaggaccgacaagggtaggtaacacatatatttttcttgatact

SEQ ID NO: 269 - DNA, 70465 bp, Homo sapiens
  source 1..70465: mol_type = genomic DNA
gtaggtaacacatatatttttcttgatacttgcagaaatgatttgttttcagggaactgtagaatttatttcagtaccctccatggaaaaaagacagggaaaaagaagtatcactctcattgaaaaatggtaagtaaaatgagaacaaataatgatgaacaattcagacaaatgagctgaactgtagaaattattcagtattaaatttagcgttatgttgaagcattatatcttctaaaatttcaggtaatcgtaaatcagattggggcattgaggtgctataaacagttgttcgatataacatatttatctttagtcaaatctgtatctacaaatttataggagtttgatacttagattaacatgtttaaaatactacctatcaataaaaaaattgattttcatttatttatattttggagggtgacactttacactttgcattaccctcccccacatttttttttgccatggggaatttgttaagttgatttaaaatcatgtttctttttatcttaattgaaatatttagctgttgacttaattggggggtggcagaaattcaattacattatttagcttcacatcacaagaagtggattactctttcttttacatttttagattaaaagtaataaagttctctaacagcttaaagtgattcatttcagaagtctttatcctcaaggtaggcttcttaattattctcagaggaaatcaaatcattcgtctaataattatagggaacctccatggtatctcttacatctgtccatttctaaccatctttactcctattttagttcttggtttactacatcaggcacctaaagagagatttctagcattaatctcagttgtctcaagtgattatctccactttgttctcaacgatctttcagtggccgtcatttttctaaagtgtaagagctttggtactataatctcattcccttgagaatgaaaccctgactccttaattgtgcaaaattatctggtcagcctcactgcttatttcttcgtgcctgccctatagcaagcccctccaattatgttctagcgatattaaatcaatttgcagatgcccaagtgttttgtactccctctgacctgcctgttcttcaggtctcacttcagatgttgtctttttctagaaatcctaaaactattagtcgctcctaacatctggaaacttggatattgtttcatcatttaatgtaatttaatatcaataaatcatttatgcatttgtaaattttccagacttggtggctaccgtgtagaatatatgatctgatgtgcctgctcctaaaacctaacacatagtaatcaatttgtttacttttctgcctcccacacaagctccttatagaaaagggctatgttttatttgtgttatatgctcaggactgatatagtgctgagcacaaagtgggtgttcaatgtgtctggactcaagcctcctctccgaaacctgagaatatttcccctttggatagtctaccagatttatctcactgcaccttccagggcaggtgcggtggctcatgtctgtaatcccagcactttgggagtccaaggcgggcacatcatttgaggtcaggagtccgagaccagcctggccaacatggtgaaaccttgtctccattaaaaatacaaaaatgacctgtgcgtggtggcatgcgcttgtagtcccagctactcgggaggctgaggcaggagaatcccttgaacccaggaggcagaggctgcagtgagcccaaatccaccactgcattccagcctgggtgacagagcaaggctccgtctcaaacaaaacaaaacaaaacaaaaaaacagatttatctcaacttccttgaaaatagctggtcccacagtatccaaagccaggcttgataaaggcaaagaaactcagccactctgtgtatatgctacaaatacaaacttaaacttataaaatggtggctcatgcctgtaatccctgtgctttgggaggctgaggcaggagggtcacttgagcccaggaattcaaggctgccacgagccatgattgtgccactgtactacagcctgagtaacagactctctctaaaataaattaacaaaaatatttgaataaacttatatcgccaccatggtgtactttctcccagcatttcacattgagacattatgaggctctgcgggctactccaatgatgtcagtttcattaccatcttttgggatgttcattccactcttgggaagttcttccccctctggtttgtcttcttatagtcagtgttttctccaagcattctaggccacccacaccatcccctaaggaccgttgaggtttcttggatagccagtgattactgtgcttctatatacacctaccagttttgcactaaagtagaaataacgctttctgctttttgaggtttctcctccgctttactcatgagtaatttttggcaaatgatccaggtattcccttctcactgggtcatatggacatttccctttcagtttaccttcaccttagacttaaacagaaacactaacgccattccacagagagggtcgctgaaatctcaccccaactctgaggacttttctggttttgacaaaggatagagtttctctagccccaatagatccaccctgtccttatccagaggttgaaattgtacttccattgacattctataggaattggtcaggagctgttaaatttcctatgacttttagtacaatctcagatacaaactgttttaagtaattgcttataagcacaggctctagagttccaacccgttttggaattctggcctttctgctatctatgtgcccttgagcaagtctcttaaatcttctgctccatacatttctgatgtataaaataggataagaatagtgtctatgtcacagggttgttatgaggattatctgacacaatatatgtggggagcttagtgggttgcttaaacacgataatggctcagttaaggttggcaattttgatgatgaaaatggtgatcttatggttctgatagtcatgatatctgttaattcattcttacatgctttcaaatttccctgagaactacagagagaagaattagactcagtccctgctttgaagagcttcacagtcttggagagaaaaggcacaaccagccagtaaaataagggttgatagactggagctgtacaagagggcaagaacaagatccctggggaatggaatgaagctatcttactaagaatggggaataagtcaaggtctatagaccattcaaggttctgagttacaactaacagaaagtgactctagttactttaggtagaattatataaattactagaaggatatcagatacctcatggaatcaataggaaagcctgagagctaggtttaacaatgggcagaaactataaaggaagagcaggctaggcatccagaatatatctactatcaagctatgaggtaaatctgaattattttgctgtcatgccagagtccattgctttaccaggagcaccattgcactggacattgcttgctatcgtcactgcaaaaaaaaagtatctaaattatttattcttgctgtgtgtcgctcattctacaactagggtctctggcagaagcatctgatagtcagaggatggatatttttttctgtaaaggggcagttagtcgatatttttgtccctgcaggtcatgtagtctctgtcacaactactcaactctgctgttttagggtgaaaaaaaccatgtataatatataaatggatggacatgttgtgtttaaataaaactttatttaccaaggcagatggcaggctggaattgctgatgggccatagattgatgactgctgttacaatacaggtttaggccacagaactaggttcttattgccagagatcagggaaaataaatagctgaatttgttcttcattgtagtggcagtcaacacctcgtgtcctactaagtcccatacaatggtagattcttgaatccttggagagatttagaagctatgcattcctgaatacacaaatgttcagtacagccttcatgtggtgttctcaacaaaaatctgctgagttttttagtaagccaatttgggttaaatcaaaataaatttgagctagtaagaagggctatattggtatttttcttaaatggtacatgtatggtagtattagctatgaccgcaataatgctgtgacacaaatcaccctagaacttgctgactgaaaacaacaggcatttctggctgtttaggttacttgtaccttgactgactcagctgggcttggccgggaagctctgcttcaagatgtgggttagtagagctaggttccatgatgcatgttggattaaggtctgctatatatgtctttgctctggacctaagatgaagggtcagtgacatgttttcatgaccagtcaccaaagtataaacctaaacctcccaagaacactcatggcctctgctcctgtgaagttcactgacatttgattggccaaagcaaattacgttgccaaatctgacatcaatagaatgagaaagtagactcttcctacagtggaaggggggagagatgttaatatttgctgaaccataattcaaattattagagatagttaataatttataacaggattgttttacagctagaaaatgccttagtggttttgccctgcttcattattttataagggagaaaactatatttcaaagatgttatgtgacttgcctaagcacatactaaatagtacatctgcgtatttctcgttcagtcatctctattaaatgtcataaggtaaaacagacattacgctttagatgagaacaggaagatatttaaaaagccagactaatttatgactgctcattcattatctaacatacctgcttccatacattgtctttcagcaaatagaatttcaacatgtatctcaacaagttacacatcatcaaagtattaaaagccttattttcacggacattctgattgttcactaacagtcaacacagtcaacagtaaatccacaagcaccatgaacgtatggatagatatgcctatatgtaagattactagttaactttttgggaaattaatattactgctaaattcagttttcattgattttcgggtgggtaactaactgcttataaaatatacctataggaaacatgtaaaaacaaagaatttacataagatctatctgggattagagaaacaaagctcttttcttctttcaatgaacacttaagaagggcctactaaatgtcagatgttatactctattgtcgttagtttctttctgataccatctttactatcctgtaagccattagtgagtgtttcccacttttagaaaatgacttcccaaagactaagagggtagaacagttcaaatgctatcatcaaaatgaaggcagtttctagactacaaattgctgtacagccataagatggcattattgtgtaacaacaaggtccctgtcaaacatcaagaaagtaatgttttaaaatcagtatttccttcttggaattctacttagtcagatatgttgggttgacttacccacccatataagtagttaaattcaagatggttctgagctttagatatgatgtaaaatagacatgagactaagagtatttatttctatattattacatttctatagcaaaaaaagaaacctctaaaaaaattagagaaagaagtataagggcatttaattaatcaaatgttcttattaataatatttcataaaataaaggaaatgttgaaaaataaaatatgaacacagtgttgtataaagtaagtcagaaataagagaacacttactgtatgaatctattatatgaaactaaaaaattaggcactttggagtttagaggtcagataggagttacctttgtgagttgtgattaacaagggctgaagcaggcttctagggactgggtaatgttttgtttcttgatgtggatgctgcttgcattgatgtgtttcctttagggaagtttatggaaccatacacttgtgatgtgtacacatttctgtgtgaatgctggactgaactaaaatttatacttacattttaaaaataataaatatgtactttaagtacaggagggacataaatcataattgttttaataaagaaggtgtagctttaggaaaagcataccgcattgttctatttttggcatttcaccatggacccctgaacattttataattgcccagtacagcctgtcagataaatatgtgggcactactgctttagagaaagtattttccaaatttatcaccaccattctggaaccacagttccacaggtcattaggatgctgagggtgggacagttggaagctgcttccccagggatttctccattgttcagttctcatagcagtaagcctgcagcattaaagcactcacaggctctggctgccatttgtgctacatgaagaaaaatgagaaaatgaaagaaacgaaaggagagaaaacaggagtagaaaccaaataaatcagattcgaagagaaagtccctaatcgtctctgtattcagacagtagggaatgagcatgcaattctgcaagcccattcctaatgtgagtttctgataagaatttgctatctcagttgtaatctttctactgagacattcaataaataattttctttcatgtcaaggtaaggaaataacctcacattacccatataaggaaagaatagtatactaatcctcacgttatcactatagaattttatcttttaaagtggaagctagagggacagatgcataatctgggagcttttccagaattgtttgttcctaaatgagacattcagctatagcagcattagcagcctcttctatttgtcacaatgccatatgtagttgggaagatacatgataagcacatacaggatgctgacaactcacgaaggattatcattgcagtcttagaattaatctctaccatatttgaagatttttatgcaagactggatcttcattccacaacccaggtataggtatatgtgccaatacaaaatacttactgttggtttctgagcttggtagaattaggaagtaaggggtaaaaaacaagcaagtcctgaacttaagaaagttgttcatatcctattccatggccactgttttactgactggaagaaaggaatcccgtggattcagtcatggggacaatgcagaagaagatacagtagacagacagagaagtacaaatttctatttatgttccacttttcaaaattttttatctttaaatgttatttttaacgtttgtgggtacatagtgtgtatatatatatatatatatatactgtatatatatatatatatactgtatatataaacacacatatataaatatatactatatataaacatatactatatataaatagatttactatgtatatacacacatacatagtgtatatatgtggggtatatatatatgtggggtacatatatatatgtggggtacatatatatatatgtggggtacatatatatatgggttacacatatatatgtgtatatatgtggggtacatatatatatatggggtacatgagatgttttgatataggcatgcaagtatcatggaaaatgggatgttcatcccctcaagcaattattctttgttttacaaacagtaaaattgtatttttttaattgtttaaaaatgtacaattaaattgttttgactatagtcaccctgttgtgctagcaaatagtaggtggtgttcattctttctaactattttttgtaccaagtaaccatttcaacctccccctcaacccccccactacccttcacagcctctggtaaccatccttccaccctctatctccaagtgttcagttgttttgatttttacctgccacaaataaatgagaacgtgtgatgtttgtctttctgtgcctggcttatttcatttaacacaatgacctccagttccatccatgatgttgcaaatgaaaggatctcattctttttatggctgaatagtactctattacgtatatgtatcacattttctttgtccattcatctcttgatggacgctcaggttgcttccaaatcttggctattatgaacagtgcttcaacaaacgtgggagtacagatatctctttgatatactgatttgctttctttgggatatatacccagcagtgggattgctggatcatatggtagccctgtttttagtttctgaggaacctccaaactattctccattttggttgtactaatttacattcctgccaacagtgtacaggagtccccttttcttcacactctctccagcatttgtaattgcctgtcttttggatatgtcgttttaattggggtgagagaatatcccattgtagttttgctttgtatttctgtgatgattagtgatgctgagcatattttgatacacctgtttgccatttgtatgtcttcttttgagaaatgcctattcgaatcttttgtctattttctgatctaattattagactttttcctatagagttgttttaactccttatatattctggttattaatcccttgtgagatgggtaatttgcaaatattttctccctttctgttggttgtctcttcactttattgattgtttcactgcggaggagctttttaacttgatgtgatccatttgtctatttttgcttttgttgcctgtgcttataggatattactcaagatttgttttacccagacagatatcctggagagtttctccaatgtttccttgtagtagtttcttagtttgaggtcttagattttagtcgttaatccattttgatttgcagttctctgatggccagtgatggtgagcattttttcatgtgttttttggctgcataaatgtcttcttttgagaagtgtctgttcatgtccttcacccactttttgatggggttgtttgtttttttcttgtacgtttgtttgagttcattgtagattctggatattagccctttgtcagatgagtaggttgcgaaaattttctcccattttgtaggttgcctgttcactctgcatcaattttgcatcaatattttttgcatcaattttgcatcaatatttatcacatggtagagttcagcagtgcagccatcgggtcccaagcttttctttactgggagagtttactaaggcttcagttgtgttactcgttactggtccgttcaggttttggatttctttatggtttaatcttggtaggttgtatgtgtctaggaatttatccattacctctagatttttcaatttgttggcatatagttgctcatagtagccactaatgagcctttgaatttatctggtattaattgtaatatctcctttctcatttctgattttacttatttgggtcttctcccttttttcttcattagtctggctcaagatttgtcaattttgtttaccttttcaataaaccaacttttcgttttgttgactttttgtatgttttctttattacaaagtcatttatttctgctctgatctttattatttctttcttccactaattttgggttcgatttgctcctgcttgtctaggtctttaagttgcattgttaggtgatttatttgaagtttttttcttttttgatagaagcacctacagctgtaaatttctctcttagtactagttttgctgtatcccataggttttggtatgtggtttttctttcattttttgaagacatttttcaatttccttcttaatttcttcatagacccagtggtcatttagtagcatattgtttaacttccatgtgtttgtatagcttccaaaattcctcattgttgatttctagtctcattccattgtggtcagagaagatgcttgatattatttcattgttactgaatttttaagactgcttttgtgacctactgtgtggtctatctttgataataatctgtgtgctgagaagaagaatgtgtattctacactcattgaattaaatgtcctgtaaatattagatccatttattctatagtgcagattaagtccaatgttactactttggttgagcttctgtctgggagatctgcccaatgttgaaagtggggttgttgaaggctcctgctgttattgtattgaggtctttctctctcctgagctcgaataatatttgctttatatatctgtactccagtgttgtatatatatttgctttatatatatatactccagtgttgagtgcatatataattgttatatcctgttgctgaattgacccttttgtcattatataatgaccttctttgtctcttcttacagtttttgtcctgatatcgatttggtctgatataagtagagctagtccagctatgtttgggtttccattggcatggaatatctttttccatccctttatttccagtctgtgtgtagctttataggtgaagagtgcttcttgaggtcaacacaaaattgggtcttattttttcatccattcagacactgtatctatttttcttttcttttcttctctctcttttttttctatctttctttcttttcttttctttcttgagacagcatctcactctgttgcccaggctggagtgctgtgacaggatctcagttcgctgcaacctccgcctcctgggttcaagagattcttgtgccttagccccccaagaggctgagattacagtcacccacaatcacgcgtggctaatttttgtatttttagtagagttggggtttcatgatgttggccaggctggtctcgaactcctgacctcaagtgatccacctacttcggcctcccaaagtgctaggattacaggcgtgagccaccatgcctggtcgagacactgtatctctcttgattggagagtgttgtccaggagctgagcctagaaatgggtcctcacaacactgtccagtgccctatcctactgtgactaagctggtatccaagatgcaagacagaatcctctttacttttcactctcctctccttaagcaaaagtaaggagtcacttttgttactgtgggctgcactgcttgaggtgggggaggaatggtgcaagcactcccttagccatgccagctggtgtctccctaggtcacatgccaccctagtcctctagctcttagcccaggctatgactagaagttatctaggaattgcagttcgtgtgtcccagactgcctctcaggtttacctgggactccagagcactttggcccacaatggtgaggcttgccaagaaacttgagttctgactgctgggatgggcgattcccttctggctggggctagttcaaatattccctccgtgcacaggcactgactgaggccagcatggcttttttctctactgtgacagcacagcagttagttcaatgtaaagtcccccagtagctgtactgtcccttccaaaagtgcaaagattccctctctgcactgcacgtccagtgctgggtgattgggaaggtgtggtgttggtgattcaagactgtgtctcctgcctttcctcaatgcctcttttagcaagatgaagttaaaaccaggtaccatgattccgtacctggtttttggttctggtgacagtgcttttctgtctgctcatagttgttacaatttgatgtttcagtgagggagattagttatataggcttctattctgacatcttgcaccactccgtctcacttttttgaattgttgaaaccatggtttatggggaaatgttctgttctggtttttaccctcaagatcttctttgaaaatcatgatttactaagtaactagttggatagaattcatttttaagaaaataatgaataagtagttcccttagatatagcaagcaatgttcttataaactgacacaccaaaacacttaccagttcacctaaacactgaaagtatttaaaaatttatttttaacctttaagtttttctccatttgagtatcttgtgcactatacgctctcctacatatgtctacaatctttggtctgatttattttattggttgttgaatgtcagaaaccacctgaattttgcttgtatatataatgcccagtagagtgcacattggtaatcaatacgacttgatgataataatggtaatgaacactataatttgatagcatcaacttggcatgtatgccagaaataaaccatcataaacttcgagaaaaacaagtctcattaatctgtcatttattataatctccctccatcaagcgtagcctctatatactgtcttttttgccatatgtaagtttcttcttgtatatttcagattagagcttgcctcctcccacacactatcttctatttcacactgctctgttcaagtgtttatgtttctgctgaaggctgtataatctaagataatccctagctattgtaaatatcattcactggccagtcattgcattttgcacaccactgagagagtgattgtgagccaaatagaggagattttgtttccacccagcaacccaaaatgaattgtgtgatttgactattgatgccaattacatggaagttttactaccagaggcaaagctttaaactgcaaaagattaaccccatatagttggtgtcatgaggtatttaatgtatattggactatcaaagcgtctccttacaacaaaccaattatcaatgaataatattcttatcttattgtgacttattgaaactctttaaaaaacatgaaccattcagcacactataccgtctaaattagatatctaaactagccttcatttacatttccttgtttagttttaaatataatttcttctgaattcttagttgttaagagcaggtggtgaaagaatttaaagaagaaaatttgttgtatttattaagtgaaagagctttttgaaatgcattaaatagaagtttgtttctgcccatcagcttatcttctgcatattcctatttatagttcaaagcacttaagagttgcagagtgatttgctttttatgatcaccgcttcatccaggatcatttgccttatatcttcaacattttcagattcttataaatcttcacagttcacttagaggttagtttaaaactttcttcagataagaaattgactagatataaaattcacataatctcagagttagccatagatcaatatccccagtctttttctcaaagtaattgaacatttttgtttcccaggatagttgtatacaagcattttcatcctcaccatagatgattcttgttcttcagtatatgaggggactgtatttgcctgcttgttatctaaataaacatttttattattgtcaaaggaaaaagactctaatacagaaagaaattttgtgtccataggactagcctcattttggaggctgatattaatatttgactttattcattgtgtatttcttggttttgtcatttcacaaatttcccttctctaaatatgcattttttctctacctggaatagatgagttttaaaagacattactgtaaaataaaactgtgctaaatgtacttttcccagaaactctctttattttatcaattataatttgctttggctaagtgttacagaagctcaaggtagaaatgacttaaataaaagagaagcctatttctttctcctgaaaaaccctggaagtttgcagccaaggcagatatgttggccccgctcatttctgttttctactgagtgagttatctttacttggtggaatttcatccacatgttgcaagatggagctctcatgacatccacattccaagaaggatgtaagaaggccagaagaagggtcaaagggtgcataccaattgtcttttagggaagtttcgtgggagctgccatgttatatttccacttacgcttcattggccagatgtctgtcacatggccacatctaagctgcaagtggggctgggaaatgtgtttatttcacatgtgctctgctaaaaactgttcagtcatgaaaaaaaaaaaaagaatattggaagagaactaacagtcttcaacatgtatatttaaaaagagcgttatttttcttttacttatttatttttttttgagtgagagtgttgctggggctggtctcaaactccctggatgaagtaatcctcccacctcagccttctgagaagatgggattacagaggcacaccactgcacccggctcctgttttgttttgttttgttttgttttatttaaggagccctaagaagagtgtaaaaagggcaactttgtctttagttaacccttccctttttttttctttcctaattttatgaactctgattcagcccgcatcttactctggcttactaaattctgccaatataagagtagaagttttcatcatttccctaggtacctactcttctgtttctccatattctcaaagtaatttctcattcatcaacttttgtctgacttagatttaagctactgcttaggaaatgtgagaaatggagaatttggttgtcatagcgctcctacttagagggctaatgctttcctaaattttactaaagttggatcctaagttatataagttacctaaatttaattagcaagttttgcttactaattcactgactaaccactataaaacaaggacctaagtttaaagctgatattatgacaatgtactgaaatttcaagacattactttttctggacacgtaggatggaccttgcataagataaaagagcttatttttaggcacacctatgtgccaggcatgggttcagagccttacatatatcatttcattctcaagagaaccttaattttagagacgaataattttgagttttagaataaataattgtcttaagctcacacatgaagtatgtgccagagtctatatcgtgaccccagttatgaattcttttccactacatagcctgcctccatggcatttcacataggatttcttatgctcaaggattatgttcttccttccagatctatagacatcagtagaaacaacacaaatacgtataatgactttttaaagtctcatatacaagaccaaagtactttaaaaagatgtttaaatttttgaaggtgatatctttatgttcgagatcagtagcagaaatggattgtaattcattacaagatgcttagcaaagccaagtggtaaaggattttctgatgtgcttgaggagtcctgacttgctccttagcaaattcctacacaccattcagcattcagtgcaaataccacctcttggaatccttctctgttcttgatcaattattccattgtctggattcatagaactctctgtttacctatttatcttacaggttattatcatgagtttatatgtcttacatagctgtttgcaacatgttaccattcagtggagtactttttcctacagggaagaggctgtactatataccttttgtcacccctaagtggaatgcagtatggtttcatgttagatgttcaatacatgttaatattttattgatgagcccttaaatgtatataataagcttctcactcaaaagctgtaatattctagaatgaataagtttcttgagaaactctttggttaacagaatctccatttccatcatagtgtattcaataaattattaatgcagggtaaatttccttatatttcctttcatttcgctatactaaggtcaaatcatgacttttaggtgtggatacagcaatatataaaataatctaagacctggatatatgtttttctaattagggaattggagatacatgaacagtctctcatagagaaaaatatatccattagtcactaaagtcagtataacattctggtgaaaggtttgcttgcttgcttgtctgcttacttgcttgtttcaccttctattctaggaggatgtttgtcacaattatgaaattctaacgctttaaaaatctgtttaccctatgaaagttgcaaattcttgcctgtaaggcaattttcacaagaattattttcatattagttttagtttatccgtggatagtctcaaattcctcttgaaaatatcagtgttctcaagggaaaactgcccattacaagccacagatgtggttttttgagagatttctgcctgaaatacaagttttaaaatattctttgtagtttcctagtatggctagatacttgtaggaaatgacaatttagatgcacatttgaaaattcttagcctttgagtaatttatatgtttgaataaatttaaactatctttgttcataaaaaggcaaattttgtctgtttgtcttgaaatgcttttttagtttcttcttcagttctttcacttgtctttctgtaatattcaaaattatagcatagtacttgaaaaccaaggatggtccagtctgtgtcctttgttttgaaggaattttggaatcatcagagatataattctggaatttctttgggtaccttccataaaatagtagcatcctgataaggacgtagatgctaacttttatttttatctcattttctttcccctatctctcagtaatgtaggagtgacccaaggtagctgtttattgggccctcaccaaagtcaatcaatattagtcagctacaaccacaataatgctgtgtgacaagcataccccaaaagaacaataagcacttatcctcatattcacaggcctactttgagtaatctctgattcagaatctgactgtaggttgaccaaacatgtctgatctatgtgtctcattctggggctcaagctggaatggcagtagatatttggggcacattcctccatggctgagttctaaagatctcagaagggtaagcagaaacacatgatggttcttaagacccagatttataactagcgttctctcttccatccatagtcaattgtcaaagcaagtcacgtggcatatccaaagatcagggatagagaaatacacttggcagttagtgaacccatttcaagagtgtgggtgtataacactactacaaaacagaactgagacttgcaacttgaaataccacaccattctagtagaattttattacagtattaattaaacatttttgataaaacacagacaactctgttagaaagcccaacttacaatttgttgatcccacttggaaaggtattctgaatccattttttcacaagttttctttctaattcaatgtgttggggtacttttattttacccagaaaagtaatattataaagcataaagttaatgccccctgtaaaaatagaatactgaaacaaactttaagtcacaattttattatttactgacttgagaatctccttgtgcttcagcacacttggtagttggatttccaaatagcatcatgactttccatacccagctgtctgctgctcaaaactaccataagctgactatcacagctcacaggatacccttgaagaagaccatctggtttatgtctcttggcaaaatttgtttaaaaacaagcagctgcatgacaaagtaagaaagagtctctgtctagctgtggtaaataagtagctttgcaatactcataataaagcatttttctaatccaagccaacatcttcaaaaggaaagcattattttcttacaactgaaagaaaaatctacagaattcagtgatgagggaaggactccattaaatttgtttcagcagcttgagatagtgagaaaaacaaaacaaaataaaaacctgtttgtgattagatacagtcttgtaataactagttttaaaagtatttatttagaatctaatacttggatattattttaattctttatattaagagatttacatttcataaaagcttgtcctttgaaccctaatactcatattgtgatggtgagccggaatgagtaaggtcacttactatatagtcggtatataataattcgttgaataactctctctcctcctgttactggcagtgaatccatacgggtcttcagcaaccttaattcatgcctcctcagaagaaagaattcgaccaaggaggcataaggcagaagaagagactgaggcaagtgttagagcaggaatgaaagtttatttaaaaactttagagcagaaatgaaaggaaataaagtacatctggaagggggccaagtgggcgacttgagatattaagtgtcctgtttgaactttgatttatggttttgtattttggcatacttccatggtcttatataccttctccccaattcttcccttggggtagggggctatctgcatgcacagtggtctgctgacacttgggaggggagcatgcacggtgtatttactggagttgtattcaagaggtgttcccttaccagtcaagtgtttccagaggaaggtcgtataccagttacactatgacattttgcctcttagtgcgcacgcgggagcccatttgcccaactcctgcgatcttatcgggaagctgctgatcaccaatttcaggtgtttttatctattgggagactgcctttccctggtattggctgtgaccaattattattttaaagacacagtttaataaccacctgactgaccatcacttgatggttgcctgacattcctggttggggtggcgggggtggggggtctcctaccctgctcatatctgactagctatctactgtaacactactattaggtatgtctgtatcccattacactcactctctattacaaaaaagagtaccttattcatttttcaaaagaatgagtgaattttctgctccagttaagtgcttttccttacctctatgtagagtgtcaaactagccatgcttaaggattgattctggtattaataatataaattcttgcattatgagtgtctcagtttgggttcttacataagcaaatcctgagacaaggatgtgagttcaaatagtttctttgggcagtgattctaggaagctgtggtaggatgttggagttgtgagaaagttaagagaaagcaacctgtaaagagtatgttatcaagccaaacaccactgtgagaactagggcttaattccctggagaaagtctgggaaggcataaaacacttgtctcagagttatcttagacaaggtgcaggagctgaggtgtttatatttcaactcttgttagtcacttttaaagggccgctcagggtgtcagggtattaatttcctcttgcaactacttccaacctgtttttgcatgtggttgtagctactttttctgcgtcaaagatagactttaggctaagacatgcagtttccggctgtaggacatcaaccagagtaccctggagtgtccaggcccaagataatgggtgagctccacctggatctgtggcaatgtgagggaggggcattattaccctagcaagggccactgcacttccctatccagctggtcactcttctgcgtatttgtgttacttttctgactcctgcattcctctggatcctgattcaccatcagcttttgtattctgaggcttgtgtggctgtgtctttctatttcttccttattgacttacttgtttaatcttttgctaatgtatacctttgatattcctatgtactcacctttggttagctctgtgaattattgagtcttgctttacagctttaaaaagatcatagccatggtttgttttttaagtgtgattcataacagttagtgacaaaattagaagtggaaattaaaggtcacgtgcacaatcctacacaagccaatttcttgaacgacataaaatacttgaatttgtcttttactatcaccaatatagaaataatttgggggtatttctcaaagtattgatttaacaaaactttatttttgtgtgaagattttttcaagtctcctggaaattattcaaattattgtctttaaagtcaaaggaaaggttaatattagggttacttttctttggtcacagaattgctggaggcttctatgacttgctttgaagcagctgactttatgcacagttttggttagaaattcactaccaattccaacattttcaaagtcctaaaagaccaaaagtacaattcttgaaatatccctagagaaagcaagaataaattagttttcactagaaatgaagacaaatttttctcattagttcattttgcctcatgatcagaattctttgccacatgaaaatattttggttaataggttgtaacataaagagtaatatgaatagagatctagcccaattagatgacagaaaaagagcaaagcttttgaaatgtataaagaagaaagtttaagagagacatgaacaaggctggatgtagtttaaggttgaagaacacagaaaaatgagataagtgaagttaagtgaaaatccttcccttactgagaagaaagagaactcaaaattatgtgaaggtttttgttttactttgttttgcttcttaataaggaaacaacagtggcaaatctggggcattaggaatggtggaacttctataacagtgatgtttcaaaggtcacaaaggaaattaaattgtagattgggtttaaaagcactggaattacttccagccccattttttcaggattttgtatgtagcaggaaatagatcaccgcatagctaaaggggagaatttaggttttaactggtctcagtgcaaactgcttcatagttcctttgcatttggtggtgtatgtaaaacatgaaaggtataaacatttattatcataactttattattatacaactatttattggctgcataggaccatgtgtcttcttgcaggtataatcaaaaataaaaagacagaaatgtatcttatatgaaggctgccattatcgctcttatcaatggccataaaatcagatttcttacatgtacaacatatgaaaatatattaaatatgaaacgttttccattaaataattctgtaaatgattttcataacatttctgtccatgatgtgtaaatctgtagatcaaatactgcaagtgtacagaacttaaaatgctttggtcaaaaaaattctcttattaatatgacaatggcgttcaaaagtaaaaaggtaaaaatacagtttagcattaataagtaaactcagaaagtaaaatatattgatcatacagtctgtattagtctgttctcatgctgctctaaagaactgcccaagactgaataatttataaaggacagaggtttaattgactcacagttccacatggctggggaggcctcagggaaaacttacaaccatgccagaaggggaagcaatcacatccttcttcatatggtggcagaaaggagaagtgctgagcaaagggggaaaagccccttataaaaccattggatctcatgagaactcactatcacaagaacagcaccatggggataaccgcccctgtaattcaattacctccaactgggtccctcacatgacacatggggattacaggaactacaattcagaatgagatttgggtggggacatagccaaaccatatcatttcatccctggcccctcccaaatggcacgtcttcacatttcaaaacacaataatgccttcccaacagtcactcaaagtcttaactcactctagcattaacccaggagtccaagtccaaagtctcatctgagacaaggcaagtcacttctgcctaggagccataaaatcaaaagcaagttagttactttctaggtacaatggaggtaccagcattggttaaatacacccattccagatgggagaaattggcccaaacaaaggggctccaggctccatgcatgtcaaatccaatgaggcagtaattaaatcttaaagctccaagataatctcctttgactctgtgtctcacatccaggtcacgctgatgcaaggtgggctcccacagccttgggtagctccactcctttggcgttgcagggtacagcccccttcctggctgctttcacaggctagcattgagtgtctgtggctgttccatgcacacggtggatcaagccctcttctcacagctctactaggcagtgccccagtggggactctatgtgggggctccaaccccacatttctcttctgcactgacctagtagaggttctctctgagggccccatctctgcagcaaacttctgcctggatatctagacatttccatatatcctctgaaatctaggtagaagttcccaaagctcagttcttgacttctgtgtacccacaggctcaacaccacatggaagctgccaaatcttggggcttgcaccctctgtagccatgggctgagctctatcttggctccttttagccatggctggagtggctgggatgcagggcacctagtccctaggctgcacatagcaggggggctctgggcccggcccaggaaaccattttgccttcctaggcctctggtcctatgatgggaggggctgccatgaaaacttctggcatttcctggagacaattttcccattgccttggtgattaacatttggctcctcgttacatatgcaaatttctgtagtcagattgaatttctcctcagaaaatgagtttttcttttctattgcatcttcaggctgcaaattttctgaacttttatgctctgcttcccttttaaacataatttccaattccaaaccatatctttgtggatacataaaactgaatgcttttaacagcacccaagtcaaatcttgaacactttgctgtttagaaattccttccaccagatgccctaaatcatctctctcatgttcaaaattccacagatctctcggacaggggcaaaaagccaccagtctctttgctaaagcgtagcaaaagtgacctttactacagttaccaagaagtttctcatctccctctgagaccacctcagtctggactttattgtccatatcactatcagcatttgggtcaaagccattcaacaagtctctaggaagttccaaactttcccacatcttcctgtcttctgacccctccaattctctaggaagttccagagtttcccatactttcctgtcttcttctgagtcctcccaactgtttcaacctctgcctgttacccagttccaaagttgcttccacatttttgggtttctttatagcagtatcccactctctgtggtaccaatttactgtattagtctgttctcatgctgctataaagaactacctgagactgggtaatttataaaggaacgaggtttaattgactcactggtttgcatggctggggaggcctcaggaaacttacaatcactgtggaagcagaagcaaacacatcctttgtcacatgatggcaagaaggagaagtgccgagcaaaggaggaaaagccccttatgaaaccatcagctctcatgagagctcactatcataagaacagcagcacgggagtgaccaccccccatgattcagttacctcccactggatccctcccatgacatctggggattatgggaactgcaattcaagacgggatttgggtggggacacagccaaaccctatcactgcctttaagatctatgaattgtttttctacattgaagaaatttgtagacatttgttttttattctctttttggtttgctgagagcagtagtctcttggaagtgagtttcatttacattttttcctttgggtgtcaaatatataaaatattttttaaaatcaatggctgagggataaatgagggttggtagaaagggaaaggataagggttgtagttaacggaccatgttttaatgtgtctataattagatggaagttgtctcttaccattcaacatcctggatggccttcctatagaggacattttcatcttcttttgtcgtccagagctagcatatgtgtttcatgtctttagatagtgaaaatcaattcagattttcatggaaaatgctctctcttcatcctaaagacagaattgtcacggctatttcgtttattgaaaagtgaaacctgctacagttagcatatagttaattccatatatgttacctgtatcatttaatacattcacaaagtaaaaaaccaaattttgccaagttcttgatagtaacaatagtaaaatggaatttctgttggcatattttcatgttattctgaaatgctgaatggatttattaagattgataacaatatgttgtacctttatgttctaaaatttaataacagttattcttccaaacatgccagcgtcctcaatagttgacagtctgcaaaatattctattatgcaatcagccatatctccttcttgagctttctttaagtacttgtatcctgtatttttccctgaaatctagcagtaagttgaagaaatgataaacaaaattgtatcattgtatataatggctatttataaatactgatgtacattttcataatgttatttaaatgattacttcagatatgtatttctgttttctacattttaacttctcaacatattaggttgttatcttagattggattttctaagacatagactctaagacagagatttccatgcagatggtttcttagtgagtgcttttaagaaaaatgcctgggagggagtaaaggcagcaggattgaggagagagaggagttgaatgatgatgcagtgaaatggaagatcttatctggtcccatgtggggttgggatgaccatttagagtattaccgtatgaagcaaggaaggtggaccattgtatccctaatccattagtcattagatgtgagcctcccaactaccagcttccgggcaatatcatcttggtcaaagaagtgcctgtctgctgagagaaatttcttggaagagcttaggtatgatctgttagtagataacaatcccagcaactgggagaatgaatgcttcatttgtaattggtgaatctcggtggcacaccacagtgttcgctacggtaatctttatacttattgagacatatattgtttttaagtaaaacttgtatacctagtaatgcatttaggtagtgtattggccaagctagtcctttttggaaacaatttcattgtggtataaatgatatacaataagctacacatttaaatagtacaatttgataagttttgacatatgcaaataccagtgaaactattagcacagtcaagataataaacatatccaatatcctcacaagtctgttttcggtaatcactctctcctgcccctcccttgattcccaggttaccactgatgctgactggcattatagataagtttgcgttttatagaattttataaaagtagaatcatgaagtatgtaccttatttctggtctgccttctttcactcacaaactactaactttcattttgtgaataatatgatctccgtgtgattacacacattaagaaagtcaataaaaacattgtaatttgattccgatactagtttatgggtctacattatgtatttaaagatactatcatgtttggattagcccttaggtatcaagtaaccctggagttgaggcagttactgttttatttgctatcaatctttttataagctcaccttccactaattacccaaaaatatttgatttaatacctgagtacagaaaaagaacatattcacaatgtttaaattttataatttaatattataatttacaacagtttaaaataactagtcctgaaattgagtatttctcacctaatattctgctctatttattacccactccacgaaaaaatttcccagaaaaatcacaaacatgtctcttgagtgtattattacccaacccacagaagaatttccaaaaacagtcgcaaacattctctcttgagtgcaaccattttctcgggccagccattcattgaaacaaaatagcctgtataacttctgtgggtagacatttgtaggttgttcttttgaggcattagtgctattgcatttaagataaagaaataacgaattcacaaattttataaagttctcttaaatttaccaagtcaatagcaccaccactttgattcctataacctgtgacttagttaggcttctcaattccaagaacataccaccattatttcttataatgtcctcagattaaaacatgtgttgtcctttctttatacctccctgcactttaattgaatattttgccatttactggattctgtacactttttatgttttcatgccctcccttttcattctcctggacatcaatcgcaagtcagtagataccttgtaagagatctctctccacccaaatcactgtttcttttgaaagagttaacctttctgaagcagcactcctgcttcacatttcccagcacaggaaccttcagtgtttctctgttgatagctttgatttcaaacgggtagccccaggagaaaagagagctgaaggatatgttttgcttggctgctttgtttgacctgcaaggaacattaaaaggcaatgaaaccgggcagagactggactgatcaacaccatcagtttagcacttattgctatactttctaaggctttacaattttatgtgtcccacgtgtcccctgaaggcatttgattttctaacctcctaggccagaggctaacatccagaattcatagtctggcacttataaaccctaacttagcttctgtcacttgtatctctatgcaactctacagcaaaatgtacttattttctctaaaatacccttattccatacttctttggagtgtaattttatatataaaagtcctcaccacctgtaaacattaggaagctttgattcgaagaagcagaagccatctccatttaactgaagtgaaatgaaacttactggagggataatgtatagttcttggaaatgaaggaaaagaaggcaactgtggcagagggagggcagcactgagagtcaggaactccagggaagttactttttggatgctgtcattaaaatgcttcagcttcaaattgtactgactctacctttctcaattctagattcaaatttctaaatgtaattggcataaattaggaacttgtcccaccagtttatagagtttacagagcactatgattataggtccccctaagatcatgcagaatgagggaagagtaatttcatccaaggaaaagcagggtaccattcaccaaagagtggagatacttattcagaacaattaatgttagatgccacaaaaaaactaaccctgaaatcaaagtggcctaatgaatgcttatttattgctcatataaagtctgatataaatcaggtagcattccttcacagtgactcagggacctaaattgctttcatcttgtggctctgccatgtcaacattgaaacttcagaggtattgacaaaggagagctgagaaattgacactaactatcaagtacctcaggcaagaagtgatgcatcacttcctacactttcccactttccaggattcaatcacaattgcactcacgaaacctcaagggaacatgagaaacataatgaaatcaataccaagatatccaaagagtactaattttcttcaccacagaagggatattggacagacaaaaaattttgggacagaggaatatccactaaatgatccttccatgccctgctcagatgctacattttccattaaggctctcttgaccaacccataaggagatgatctccaactcctctgaacatctggaacattgcattctacttatttttactgtctcctattgtaataattgttggctattccttattagacaaattataagcttttatgggacggtgatcaaaagtcctaatttatcttgtcttcctacagcattcaaagcattgtagaccttcaatacatagcagtcttcgaagccagagtattagtattttttctaaaactgtattcatcgcaacgaactcgatggatgttcaataaaaatgtattgaatagtcattcatgcagatagcaaatttcttaaaacatttcttactcaatgtttacttatattcatcatgaatataaatttaatataataatatcaaatcccattacatttagtacaaagtaaatataccaataattttatatatgtttaagtgaatttttatatttcatattatttgtgaatgatacacatcgtcgtggaaaatttacaacgttgaagtaaattatacaatatagggtatttgtattaaaatacatttcatagaaggtgctttaaatttaaaaagttagcatgaatacatattaaatgtaaaagagtctttaaaatgaaaaatatttcacacttgtgggtttgatctaaaaatatcagtcgctatatgatatggctagacagatttgagtaggtataattttaatgaataatattgtgtgatattttagggtccaaaattgaatgtttctattaagtaacagatatttaaatgccaagcagaagctacactggcaaaacaagatttagtatgttatcttaagtgtatcctaaatcctctttcatcttatggagaagaaatacatgcttttagtttccagccactcctgtctatattcatttaaaataaactattatttatatcagatatgatgccatgtgagtccacaatttgtagtatagagaaataatttcattgaattattttggctgtttgtacctgagacctgattctgaatcatttatttttctatttaagttgtgctattttatgatcctacctatggctatgttcaaaacaatgacaaaatatattttaccctgatctttttttcattattatactttaagttctgggacacacgtgcagaacgggcaggtttgttgcataggtgtacacgttccatggtggtttgctgcacacatcaacccatcatctacattaggtatttctcctaatgctatccctcccctaaccccccatcccctgagaggccccggtgtgtgatgttccccgccctgtgtctatgtgttctcatcgttcaactcccacttatgggtgagaatatgtggtgtttggttttctgttcctgtgttagtttgctgagaatgatggtttccagcttcatccatgtccgtgcaaaggacatgaactcattcttttttatggctgcatagtattccatggtgtatatgtgccaattttctttatccagtctatcattgatgggcatttgggttggttccaagtctttgctattgtaaatagtgctgcaataaacatacgtgtgcatgtgtctttataggagaatgatttataatactttgggtatatatgcagtaatgggattgctgggtcaaatggtatttctggttctgtatccttgaggaatcgccacactgtcttccacaatggttgaactagtttacagtcccaccaacagtgtaaaagcgttcctttttctccacatcctctccagcatctgttgttgcctgacttttgaatgttcgccattctaactggtgtgagatggtatctcattgtggttttgatttgcattcctctaatgaccagtgataatgagctttttttgatatgtttgctggctgcataaatgtcttgttttgagaagtgtctgttcatatccttagcccactttctgatgggtttttttttttcttgtaaatttgtttaagttccttatagattccgggtattagccctttgtcagatgtatagattgcaaaaattttctcccaatctgtagtttgcctgttcactctcatgatagtttcttttgcagtgtagaagctctttagtttcattagatcccatttgtcaattttggcttttgttgctgttgcttttggtgttttagtcatgcagtctttccccgtacttgtgtcctgaatggtattgcctaggttttcttctagggttttgatggttttcggttttaggtttaagtctttaatgcatcttgagttgatttttgtataaggggtaaggaaggggtccagtttccgttttctgcatatgactagccagttttcccaacaccatttattaaatagggaatcctttccccgttgcttgtttttgtcaggtttgtcaaagatcagatggttgtagatgtgtggcattatttttctggcctctgttctgttccattggtctatatatctgttttgctaccagtaccagtctgttttgcttactgtagccttgtagtatagtttgaagtcaggtagcatgatgcctccagctttgttcattttacttaggattgtcttggctatacaagctcttttttggttccatctgaaatgtaaagtaatttttcctaattctgtaaagaaagtcagtggtagcctgttggggatagcattgaatctgtaaattaatttggacagtatggccattttcactccactgattcttcctaaccacgagcatgaaatgttttttccatttgtttgcgtcctttcgtattttcttgaacagtggtttgtagttctccttgaagaggtccttcacatcccttgtaagttgtattcctaggcactttattctctttgtagcaattgtgaataggagttcacccacgatttggctctctgtttgtctattcttggtgtataggaatgcttgtgatcttttcactttgattttgtattctgagacttggctgaagttgctcgtaatgttaggggcagccagagagaaaggctgggttacccacaaagggaagcccatcagactaacagtggatcactctgcagaaaccctacaagctagaagagagtgggtgccaatattcaacactcttcaataagggaattttcaacccagaatttcatatccagccaaactaagtttcataagcaaaggagaaataaaatcctttacagacaagcaaatgctgagtgattttgtcaccaccaggcctggcttacaagagctcctgaaggaatcactaaacatggaaaggaaaaaccggtaccagccactgagagaaacataccaaattgtaaagaccatcgaccctatgaagaaactgcctcaactaacaggcaaaataactaaccaacatcataattacaggatcacattcacacatgacaatattaactttaaatgtaaatgggctaaatgccccaattaaaagacacagactggcaaattggataaagagtcaggaccatcagtgtggtgtattcaggagacccatctcacatgcaaacacacacataggctcaaagcaaagggatggcggaatattttccaagcaaatggaaagcaaaaaaataaaaaattaaaaaaaataaaaaaaaaaacaggggttacaatcctagtctctgataaaacagactttaaaccaacaaagatcaagaaagacaaagaagggtattacattacataatggtaaagggagcaacgcaacaagaagaactaactattctaaatatatatgcacccaatacaggagcacccagattcgtaaagcaagttcctagagacctacaaagagacttacactcctacacagtaatagtgagagacttttatactccactgtcaatattagattaacgagacagaaaattaccctgatctttaacattccaggcaagtatgttttcagtcatacatagtacgtgaatctgttatattttaaatccaagcttttggaggacaaatgatttcacagttatacaactcagcctctcccagactcaagtctctcacttgatgcagtattcccaccccatcttcaataaaagaagaaagcatgaaacatcgcattgatatagggaaagtatatctgagttttctatatagcatataaagaagtaaaataactctgtgatttgtgatgagatagatttagctgcaacaatgaggtctggatattaatattggattaacttcctttgagattgcaacttccatgtggcattcagcactttccttgttttctcatatgtaggccgagaataaaattatcctgtttctatttcccaacatgaaaggtcttttaactgtttgaagacatatgtttcatgtgaaaccaaggaattgctactgcaattttgtaagacagaagttagcaaacttattctgtaaggaacaagatagtaaatattataggctttgcgggccatatggtctatctcacaactattaaactctgccatttttttttttttttgcaaaagcaaccatagacaatatgtaaatgaatgagtgtgattatattccaataaaactttatttttaaactacaagactagttcttaaaataaaatttaacaaagaactattatatgatccaataattccacttctgggtatatacccaaaataattgaaaacaggtattcacacagatatttgtacacccaagttcatagcagcatattttactgtagagaaaaggtagaaacaaatgtacactgtgtacactgaccaagaatggagaaacaaaacgtggtgtatccatacagcagaacagtattctgctttaaaaaggaatgaaattctgtcacatcctaaaatagggatgaactttggagatattccacctagagtagttaaattcttagacaaatagttgcatggttgccagggaccagggaggggtaggggaaatgagcagttcttgtgtcattggtatagagtttcagtttagaaagatgaagagttttggagatagatggtggtgatgtttacatatccatgtgaatgtatctaatgccactgaactgtatacacttacaatggttgaaatattttgtatattttcccacagttaaaaaagacaagcccatgtgcccaaattgctgatataaatctgtattcctattagtgagcagtaatgagccatgtttagtgcagctgatatggttaagatggaacaattgcatgtaatgcaatggtgggtaacatctaataaaaattggtactcaccaatttccatttccattgtggatataaacacccttcacattatgaatcatgttatttgttatttgtgtacttacttattaaatcacttttctccatctctctcacctctatgactctgtacatgtacaaacaacatacacactgtgaacttctttagaatgacagttacattttcgttacttgattcattgtttggccaagtagatacccagtaagcataactagcaagtgaataatgtatatgtgatcattttccttttcaaaaaccacttcttatctctaccacaccaccacacattattatatgctacacttcttatataaaacaaaaaagaataggccaggtgtggtggctctcgcctgtaatcccagcaatttgagaggccgaggtgggcagatcacttgaggccaggagttagagtccagcccagccaacatggtgaaaccccatctctgctaaaaatacaaaaattggccaggcgtgttggcgcatgcttgtaatcccagctacttgggaggctgaggcaggagaatcactggaacccaggaggcggaggttgcagtgagctaagatcatgccactgccctccagcctgggcaacagagcaagactctgtctcaaaaacaaacaaagaataaataagctttaataaactcttttgcagaagagttttcatttttaaacatagaggaaaaagaagatgaggatgaacgatagtgatgcccagaagagaaaattgtgtaagacaaaatcaattgaccaggcgtggtggctcacgcctgtaatcgcagcactttgggaggctgagacgggcagattatgaggtcaggagttcgaaaacagcctagccaatatggtgaaaccctgtctctactaaaaatacaaaaattagccgggcttggtggtgcacgcctatagtcccagctacttgggaggctgaggcagaagaatctcctgaacccagcaggcggaggttgcagtgagccaagatcacgccactgcactccagcctgggtgacagagcgagactacatctcaaaaaaaaaaaaaaaagattaaatcaattttagattgatatagagataaataattccctaagtacaattaaagcaaggatcagaccctgatgagtaaggatgatctaacaaagggctgacaatataaagtgaatgcgattgtcaccaaataaacatctcccaagagctttgggacaatcttcagctaagcatattctgcactttacatcgttctgccatatgccctatgtagcttttcatcctccagaaatttgaatagcacacagtattttttggcaccgagtaattgtggaacttaaacttcctacaaagagagaagctgtttcagtcagctaagtgaccagttagtaagcaacttctattctcttagtgtcgatagggttaaagacagtcataagctccccaaacccttggccgcctcaattttttcatttaaatacttatcttcttatcttcatctattttttagagtcacacataaactctgtctatgaggttcttaaagacctggacaatatatttaatctctgtatccttaaatctcaccatgatatctcaaagatggtagataaaactgttttccctcacttctctgactccataccaatacttaatcgatcgggaaagttttataaaccatttaccgttgaatataaatatatatacacacacaaacatatatatatttatatatatttatgtatatatatatttatatatctatatatatttatgtatatatttatatatatttatatatatttttatatatttatatatttatatatatttatatatttatatatatttatatatatttatatacttatatatatttatatatatatttatatatatttatatatatttatatatatttatatatttatatatatttatatatatatttatatatttatatatatttatatatttatatatatttatatatatatttatatatttatatatatatatttatatatttgtatttatatatatttatatatacacatatttatatatttatttatatatgtttatatatatatatatatggcactatatatcagtgagcaaaggcatcataatctttatttttatttctgtattcatttattcaacatatagtaattctgatcctgtctcaaaaactctacagaaattaacaaagaacacattgtttctgcctttttggagctgaatgtttagtgggggaaaatgtattttaaggcaataatcatatacatgagtgtatatttacaagccgcagtaggtgctatgaatgaaaagtataaggagctatgagatattctaacaaaaagaaatgctgcagtctagagactgagagtaattatctgtgaggaaaagtattgaagcagagacctgcaactcagtaaagagtgtagggagtcacatgaacaaagtcttgagatgttggtagagatcttggaatgttcgaaattctaaaagaaagcccttgtgtatggggtatggtgagagagggagagcgtggcaagttttacgttgtacaagtcattgtaggccacaaaaataagtatagattttatctaagtgcaacggaaaaccattacaagtttattttttgattcttaaattttgttttacttatttatttatttcaagacacaatctcactctgttgcccaggctggagtgcagtgacatgatcacggctcactgcagccttgaactccttgggctcaagcaatcctcccatatcaccctcctacctcagcttcctgggtagctgggactactacaggcatttgccactatgcctggctaactttaaacaattattttgaagagacagggtcttgttacgtttctaagtttctaaggctgatcttggattactgggcccaagcaatcctcccgcctcatcctcttaaaatggtgggattacagatatgagccacctcacccagcaaggtagtgacataatcattgtttttgctgaaaacaatgtactttagggaacctccctgaaaacagaatggccacttagaataccaaagcatagaccaagccttgagatggcaatggaagtggaaataaacatgtctttgatacttatgttaatggagctattagtagcaacttttgacagatttgaaagtggagttgaaagacagtgaagtgtcagggatgatccctgagcatattagataatattgagctggagaacagaaaagaaggggcagatttggaggtggagacctttctagatttatattcaatttgagatacatctgaaacatctacatgaaacgtggatgaaatatctgtgaccaagatacatacgtgggatacctaggcatgtaaataatatttaaagttatgtgagtgggtgcaataacctaaagagagactgtaggtagaagattgatcaggacaaatctttgaggaacttttttttttaattattattatactttacgttctgggatacaggtgcagaacgtgcaggtttgttacataggtatacacgtgctatggtggtttgctgcacccatcaacccgtcatctacattaggtatttctcctaatgctatccctaccctagccccccacccccaacaggcaccagtgtgtgttgttcccctccctgtgtccatgtgttctcactgttccactcccacttacgagtgagtccatgcagtgtttcattttctgttcctgtgttagtttgctaagaatgatggtttccagcttcatccatgtccctgcaaaggacatgaagtcatcctgttttatgaggtacttttaatgcatttagtgactgggtaaggaataaccctgaaaaggagcacaaaaaccatcgaggtatgaagaaagccaggagatgtagtttcacaaagcagccagtggacagtaatggacaccagcataagcttaagacagcagtgatagtgttcatcagtgctgaaaactcattggaagtggctgaccatatgttgattgcgaatgcaatgccttctcccatctcctattgcttttcactgtttccgtcatgtccagagtgaaaacactggggaggcagaactgtgtaccaagaaaaatgttttgtgtcagaggaaacaagagctcattaatttccttttgacccgcatggataggaatacgataagcaggagaaaaataaaacctgctaaactaggcaaataaagcttaatagaaaagagagaaaatattgcttacaaagggaagaagacatttgccacaatattagctaaaaagagagtgaggaaaatccacatttaccatttttattatgtgtttccaaagtcgacattaagtgaatattatcactttgaagacagagaatagattgtaagcagaagaggaagtgggattttttaaatggcatatatttaaatgttgaaaaacccatttccaatactcccactgagatatatgcaataaaaacactccgcaatcagtaatatacatgtgaagctcctaaaaatcctccattgagaagattctataaaactataaagtattattatttctgctatcagaaaataaacacatactttctaagaacaagatgaagaccttaattcatcttcccaggaatatacttgtgtacttaatggttcaaggcgtttgtgatgcaaaaaaagtgggacattatgtacaaaatagttttaatcagaaaatgaaggaagtttggtatggtatggttctagggttgcaagttttcctgcaaaaagggagtacttatggctaggtgatgcagccaaggaagcatctctgaggacaggttatcctggctcctattccaaaatatttaggtctccttgagtgttcgatccttgaaccaaggacaagaatagaaagaaataaaagccacttgtataaaggttgccttcattttgtgtcgaatattgacaggagaaagccagatttgtagcttttccagggataagtgagtctcagacacagttaagagcttggctttggaattagattgacctgagtttgaatcgtggctccatcaatctctgattgtgtgaatttttggcaagttttagctgaacctcaattttctcataagtaaaataggcataatacagctatgattctgacccttgccattagcactctgagctacttgtcagtgtatcctggtctcttcctcaagcacttttccactttgttatttcaagattctttgaaattatgaacagtagctcttgatcaccaaacaaatgggagaccatagattcaaaggaacattttagtaggatatttgataagctttgtatttttttgtgctttgaaaattagagtagatttagatttaaggaactataaagctatatgtttacaaaactttattcagttgggcagttctgactcagtaactgaaataaaattgatgaaaacaaaacatatgttttactgttgtgttcacatttaaagattatttaatgcataaccaaaataggttcagcaaatgtttatacatacatgtacttagatatttgtaattcttatggtagagcatgggaatgaatgagaattctcctaggctggaataaaactaatgtctaactaagaattggcctttcacttcacgaatatatatcattatatataaaagattcatcctttgttaacttatttgtttagcttagtactttctaagtacttttctatcctgtcttggtccctatataagaatgatactattataaaaatgaacatcctattatttcataatgattagcatatcagcatatttaagagcaatgcctatatctttaccttcattattattttcatgtttaaaatcctgataattctagaaaaacaaggttgtaattatccattcataataaaagtcacaccaagttattcttgataattaacttggcatagtggagtgcactgagcaattaataattaattacgtggttactagctcaaattttattgagaaccaaggagttaaaattaatagtattgtgtttgcaatgagttaattactttgtttctttgtttttttttttggaatagtgccataattagtatcctaatagaaacagaatataaagtgggtttctgagtgcttgcttgtcgaggtgtgtaacaacatatattttaattataatttgagtcactaattgttgtcaaatacaattttaatttcttagttcagagattaaaagatatgtaaacagctccaaaaaagaatatgataagatggtttatacaagtaaataaataactatatgacttcagaagttttatttaacaacagttgtataagtgttttaactaatgttaacaatgccttttttgtaaatctcctctaccacaaatatgagataaatttaagaacggcattttcattaaatattgattattaaaattcatatataagtattggctgtacctgagcatttacagtgaaaccatgaaggactggcaggtttggggctgtttgataatatccttccttaatctttcttcgtagcatcattgtttaattttggctgaccacaatcacttatgaaaagaaacagctcaaataattagtataacctctttctcttcataaaaccttcaaatcaatgtggtcataattatgaaagtgtaagattataaaatcttctcatttaaatatacatcttcatattagcagacttggctaatttcttcatttatccttgacatccacacttaaactctgtcaaactggtcattcatagactcattattcattcaacaaagattagtgtgaccctactatgattctgagggctagaaatataaaactgaataacatataaaccctgatttctgtatttatatatacagaaaggaattatgaactcacagttttaatgcacagtgacgagtcctcagttcatgtctgccttgggagctaggcatataccaaagcaggactgtctaacacagtttgggggtctcagtctggaaggatccgggtgagatgatccttgaattgagttctaaaaaatgaggagaaattatccagataatggaaagtttaggagaaattaaccagataatggaaagtttaggaaagaaacactatttatatgtcaaggacaaagtttgaccaaatgtgtctctgaatctaactggctttatgtaagataaaattaaaaaataattgtcactaaggtttttcacatctgcagcaactaccatcatatctggctcaatatttaaaagaaatacttaacgatttttggcatttattgctactataatagattgtgctatttaaagagaaattctagatacatgccaaagtattttcaggtgaagtaatgtgattcgtgggttttgttttgacaaacttccaaggaaattatttttaatataaagagcaatggaggaagattaaacaagattcgcaaatgttgataattgctcaaggtggtgacaggtgcatgggggtccataatatactgttctctttacttatatatgtctttgaagattttcatgatgtaaaattttaaaatcagaattacagtaatacctctttctttgacaatcgttttcaaacttggttcaacaactgaatttaaattacctacatggctataaaaataaattagatatcctggttatttggtctggggtgggtcctggccatatctgtagggtgtgtgggtgtgtgtgcatttagcttcacatttgactctgaagtacgtggaaaaccaattctgggctctacatatgcaattccaatgcctgacattaggtggtgctggttgtgagccatgatttcaggtttgacttgtagagttaacagaagtgatatcaccaccgatcccacagaaatacaaactaccgtcagagaatactacaaacacctctacacaaataaactagaaaatctagaagaaatggatacattcctcgacacatacactctcccaagactaaaccaggaagaagttgaatctctgaatagaccaataacaggagctgaaattgtggcaataatcaatagcttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcataccgaatccagcagcacatcaaaaagcttatccaccatgatcaagtgggcttcatccttgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacatatctcaaaataataagagctatctatgacaaacccacagccaatatcatactgaatgggcaaaaactggaagcattccctttgaaaactggcacaagacagggatgccctctctcaccgctcctattcaacatagtgttggaagttctggccagggcaatcaggcaggagaaggaaataaaaggtattcagttaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacagaaatcacaagcattcttatacaccaacaacagacaaacagagagccaaatcatgagtgaactcccattcacaattgcttcaaagagaataaaatacctaggaatccaacttacaagggatgtgaaggacctcttcaaggagaactacaaaccactgctcaaggaaataaaagaggatacaaacaaatggaagaacattccatgctcatgggtaggaagaatcaatatcgtgaaaatggccatactgcccaaggtgatttacagattcaatgccatccccatcaagctaccaatgcctttcttcacagaattggaaaaaactactttaaagttcatatggaaccaaaaaagagcccgcatcgccaagtcaatcctaagccaaaagaacaaagctggaggcatcacactagctgacttcaaactatactacaaggctacagtaaccaaaacagcatggtactggtaccaaaacagagatatagatcaatggaacagaacagagccctcagaaataacgccacatacctacaactatctgatctttgacaaacctgaggaaaacaagcaatggggaaaggattccctatttaataaatggtgctgggaagactggctagccatatgtagaaagctgaaactggatcccttccttacaccttatacaaaaatcaattcaagatggattaaagatttaaacattagacctaaaaccataaaaaccctagaagaaaacctaggcattaccattcaggacataggcatgggcaaggacttcatgtccaaaacaccaaaagcaatggcaacaaaagccaaaattgacaaatgggatctaattaaactaaagagcttctgcacagcaaaagaaactaccatcagagtgaacagggaacctacaacatgggagaaaattttcgcaacctactcatctgacaaagggctaatatccagaatctacaatgaactcaaacaaatttacaagaaaaaaacaaacaaccccatcaaaaagtgagcgaaggacatgaacagacacttctcaaaagaagacatttatgcagccaaaaaacacatgaaaaaatcctcaccatcactggccatcagagtaatgcaaatcaaaaccactatgagatatcatctcacaccagttagaatggcaatcattcaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattactgggtatatacccaaaggactataaatcatgctgctataaagacacatgcacacgtatgtttattgcagcactattcacaataacaaagacttggaaccaacccaaatgtccaacaatgatagactggattaagaaaatgtggcacatatacaccatggaatactatgcagccagaaaaaatgatgagttcatgtcctttgtagggacatggatgaaactggaaaccgtcattctcagtaaactatcgcaagaacaaaaaaccaaacactgcatattctcactcataggtggggtgggaattgaacaatgagatcacatggacacaggaaggggaatatcacactctggggactgtggtggggtggggggaggggggagggatagcattgggagatatacctaatgctagatgacgagttagtgggtgcagcgcaccagcatggcacatgtatacatatgtaactaacctacacaatgtgcacatgtaccctaaaacttaaagtataataaaaaaaaaaaagttcgttcctcttgttagaattttcatacaaatgcaagactccttattttcaaagagaaaacaaaattttatataaactaatacctgagaagaataggaaaaattaatgatgtatgggataggtactgataagaacctcttcttttgtgtacattgtgataaactaatgtttaacttgagtcgatgtgctggaaatacttgttaaattttcaagaattttgagatctgttaaacacaaccattatcaaaaattaaattatggtaacttacattaaatgaaatatattgttcacaaatgtactagatgcacaaaagtcatcactacctaattcttttattatatttcactattatctacgggtttgttacctgtataatggaatgactgtataatggtgtagtgctaacttcatgtttggtgacattataatgatagcttaaaattaaacatactgggaatattcaaatatgtccttcctctcaggggatctgattgttagacatttaccagcatagcactgtcttaattaacatcatataattgtcttttatccttaagatgttgagccagtaaatatcacaggagagtttgttccttaggttttaatacttgctttaccagtttactgtagcatcatgtgaaaccagcttctagaccagctgtctttaattggtatgtacccaagctaatccttgtcaggctatcatcaacccaagcacttttgaagttatttttattattacgctacagaaagattttatggcctttttgctcttttctttaccctagccctaggatcatataatttcatctcctatttattcctatttatttccttatctaaaatcttacttaactagcttgcttgcctgcctgtctgcctgtctgcctgcttgcttggtttttctttttttctttcttttctttacttctgctttcctttcctttcttttgtttcttccttccttccttccttcttcctttcctttccttctgttccttttccttttccttttcctttgcgttcctttgctttccttgctttcttcgcttgctttcttttcagtaccacaaaaaaactaacaataacaaaaatattgctacaaaaattgtgacctcccaattctatcctcatttcacggacccataaaatgattatatatctatgccaatgcgttctaactacatgatattattattgatgataagagaatccaaccctcaagaaatgaagtattataatgttttagaacacacagaagcatttttaaccaaaatgttccatgtgattttatagggtgcatgttttaccttttcttatttggaaaagtagcaaaagtattaacaaataaaataagttacatactctttgtatataaatctttcgtgtacataataatgccatttcccttctagtgcttccattattgaaaaaaaatagacatatagcaacgtgctagacattgaagttaactgttaaaaagtcatcagagggtaatgttaaaggtggtatttcaggtaataaagcatatgcagaatgattctattggctttatttccaacatgagtgttattagtaccaaggacactgaagtttaaaggaggggagtgatgagactgatcattactttgtcttctgtaacaggacttaggtggcccagagtgatccttgttggttgattggttagttgaacttctatttttgttactaggcctgcagatctgtggaatctccagagtgctgtgtaatgttacataggccacacaccagacactgttccctgaaggactgaaggtggaattggaacaatatgtagacagttcttgttgcagttaccaaggaaatttggttaggattcaggcctccatgtcagaaaaggaagtctattttatgtgctagaaaaataaccctaatcatcttatcttgggtctctctggggagcatgatgatctacctggtcttgtaaactgtatagaaccggctgggttgagacacatatgtctcataacagagcttccacttaaggctataaaatagtggtccctctattgggtatatgccttatggaaaaatatacgttatgctggtacctattataaaaaccaatgcaaaatgattagagatttctaatccaagtgttatcctcatctgctcccaaaatacagcaatgaaaaaatagaaaataaaaaaggatatatttacagaactgtatttacagaaatatatgttgatgacaagataatcaaatacatatcaaatcatgatgtgtatttacagaaatatatttacagaaatgtaaagcagagaatggggcggaagtccctgacagacttgacactgaaccacaaaacaacagtggaagccaggaatttggatgctgcgggatagttgggattaaaattgcttcaagcaaggtgtggaactggagacaaactcagtattcaaaaccagaggctgggctgtggtgcttgcattcgtgaaagaaaacctggggaaaaagtgcttcttcaggctgtagcctgacgctatggcttgcctaaacttatgggcctatgtaggtgtaagaataaagacatagccttgctgctgggaactgtgttgcatctgttctcttgcttgatgaatggattgaaattatccccagtaacattgcagtgcaagaactttaaaccttaaaggggtggctggagaccacgggacgcgatgcacatgcaactactaaatgctagatgaggatacctgaggagagagaacaagacacaaacgtgaacatggacctccaatagaaatgacaatcgaaaacatacatttatcttctctctctcccaaattgttcaaagattacgctaagggaataaaaacaatacagtcataaaatgacaaataaaatggaagaagagacatctgcacagacatatcaagtcaatgagatgaaaattaggacaagtaagccaatttgtttttcaggatcccagaaaaacttgtggattgaagacgctaggtgttttagaaggccatatattcccgagaggggctggaaataaaggggttgttttgaatcccttaaaaggggcaattacatttcatgtccccagctcagggtagttaattagacactactcctctctcatcccagcagacgtgagtgagtggtttgctttctggtgaggctgaaacaacttttggcatttggacaacaggattagttgatggaagagagagccaccatactgaaaatacaggattaagttaaagtcaacatatttaatggtgagaccagcagtccctcttcaccttcttggctcttaagtttctatcctctgggcagtagattataaatttattctcctgagagcctacctgcaatacgaaaactatttttagtcaagttttattgctataacaaaataccatagactggatgccttataaacaacagacctttatttctcaccgttctggaggctgggaagttcaagatcaaggcactggcaggtttggtgtctggtgaaagctcatttcctgtcttctcactgtaacctcacaaggcagcaggtccaagggaactgtcttgggcctcttttgtaagggtactattctcattctggatggctttggccttatgacctaattaccttccaaaggccccacttcctaatgtcatcaccttgagggtgaggatttcaacgtataaattttgcaagaacacactcagatcatagtaacctacaattaatgatatgtaatgatccaccagtgaaatagccagatctgtgcctaatgaccatatattgaagcccaccaagtataagccctacatacgcacaaggaatttccaaaagcatttaatgcctcagttttaagagacaatcaaggatttccagatatttgaacatgtctcaaagatggaagacagaaaccaacataaaaacaaaaagcagaaatattgggaaaaaaacaatagccacataaatgaaacagaaatctctgcagaaagagagacaaggcatagaccttaagaaaactttttaaagatagctataaaattattatctcacagtgagactagtcactgtattcataagaaagaacaagagactatagaaggtggtatttccgagaaagataagggttatttaaattaatagtatagcaggttaagtttttaaattcttaattgtataaatttatgggttaccagtgtaattttgttatatgaatatattttatagtggtgaaggcagagcttttagtatatccaccactggaataacatacatggtatgcatagcagttttagagcaatgaaactactctgtgtgatactataatggtggatacctttcattatatatttgtccaaacccatagaagatacaacatcacaagcaaactctaatgtaaactatggacttcgagtgttaatgatatatcaatgtatgtaagtcagttataacaaatgtaccactctggtgtgggatgttgatagcggggtaggctgtgtgtaatgaggaagggggccagagagtatacgggaaatctctgtactttcccctcgattttgctataaatgtaaaattgctttaaaaaatcaagtctattaaaaatgaagagcagaaataaaagttcaaaagtagaagataaatttgaagaaatatctcataaagcagtataaatagccaaaagcatggaaaatgggagagtataagaatcttagaagatcattctaggtaagtgtttcacaaatagaggtgattttgtcctgcagaggacgtttgacaatgtacggagacctttttcgttgtcacaaataaggaaaggatgtactactggcctcttgtgaagagtgacgcaagaggaatgcaccatgcagccatccacaaccaagaattactcatttcaaagtgtcaatagtgtttatgtttagaaaccttgttctatgtaatccaacattcaaataatagatgttaaaaataaaacagaagagaggcaactgtggagagaaaattatcaaagaagtgacaacatcagttttcttagaactgaagaaaatgagtgtccaggtttaaagggcttactaaatgcccagcacagcaaatgagctaaaaatctacaccaaggctcatcatcataaatttttacaaaaacaaggacaaagagaagttcacagatcagaaatcagaatggctttggacttttcagcaatagccatggaaaatagcagaaaggaacacttcctttaaacttctaggaataattaactttggctttgatttcatatcctatatgaaattatgttcacaaattctgtgatgctcttctattcaagagttacagctaaatgtccctgtcattgagtgtggactagattcagtgactcttttgtgatgatatgtcatttccaagtgtaggttgtagaaagactgtggtttatgtcttgtgttctccatccacgcctatctctctctgtgtctctctttctctctctctctctctctctctctctctcatgcattgcttcttctgtgggaacacatgtcatgagcagccctatggagatgcacgtatgctgagaaactaaggcctcctgcaaagagccacaagaatgagcttagaagtgcatcatttaacaccagacaaatctcgagaggctgcaagcatagacaacagcttaactacaacctcataaaataagctatgccagtcccaccctgctaagccactctcagattcttgaccttcagaaactgccaaataataaacatttttgtttttaagctgctaaattttggaatgacttgttattcaacaatatataaattgtacaccctgataaactataaattgtgcggagaaaataattttcagagatagccttatgtactcccctaaaccttttttccccagaaatctattttcaaaaatatactctaccaagtgaagaaagaaaccaagaaagacaaagaatgtttagttgtgattggagatcagaaggagagtgttttaggcatgattcgccttgttatgctgcaataataagcaattccaaaatattagtggctagaaacacaaaggttgattttgtatctttgcaacatatccaacacaagtcagtcaagggttctgatcattgtagtcactcaggaatctaagctgattcagtctctagtattctatcattctaccgtaacaataagaagctttagcgttcaccatgtcagtcaaaaatgcgtggaaaattttaaactgactcttacatgcctctatgtaggagtgatatatgtcattttcatttcacttcatcatccaaaggaagtcacgtggtcatgcctaactctgaggggacaggggattataatcactaatatttttaagagaggtaagagaattaggaaaatattggagcattagtattacctgcaaagcctaggatccggaaaacagagaacccaacgtgggaggaagatgcaaagtggtgccagaaagatggtggaggagatcctgtaacaacagctgtataacagttgtagagagaaacctgtacagattggagaagaaaatagtgctccaggaattttgttaccaagaaaaaaaaaatctaatgtattcgaatgtattgagaggtggtattcttattgtgtaagagtataatatatgacatgtatgacagacagaagtaccatttaaaattttattaaacataagaattagatccctatctcacactgtacataaacataaaaatggtaaattgatggattgcctaaacatgaaaggcaaagcattaaaatatataaaaatattttggggcatgtagggcaaaagaggtgtcccttaagcaacagcacaaagcaaaaattgtaaagaaaaagacatatatttgatcattttaattaaaaacctgtacaatgcaaagacttcataaacaaagaaaagacataggctcagatagtatatttgcaatactaaaacaataaaggatttatgtcataatttaagaaaaatatttacctatcaatagaacaaaataaaaataacctaacaggaaaatatttttaaagtttaaaaaataatagacagttcacagaacatctgtatgtcaaatacacttatgaatagatgctaactctcatttattattggaaaaattacttcatattagtcggttttcatgctgctgataaagacatacatgagactgggtcatttatacaggaaaaaggatttaatggacttacagttccacacagctggggaggcctcacaatcttggcagaaggcaaggaggagcaagtcacatcttacatgaatggcagaaggcaaagaaagtcccctttataaaaccatcagatctcatgagactaatttactatcacgagaacagcatgggaaggacctgcccccatgattcaattacctcccacgaggtccttcccacaacatgtaggaattcaagatgagatttgggtggggacactgccaaaccatatcgtactaagtgagataacgtactcccatcggactggaacaagtaaaaaataaaataagattgtgaaaggtcacaaactctcattcttgtagaaatttaagttggtacaaccaatttagagtgtaatttagcaataatctagttaaactgaggatggcatgtcctaccaaacagcattccagttaatcacattgaagatagcatgattgttctcaggcttccatattttctgttgaacggtaagttgtcagtctaattggcattcctttaaaatagacttgtttcctcttgatgattttcacattgtaattttgtctgattttcagcacttcaccggtctttaaatgattatgtaagattttcattgctgaggaggaaatcctatcacctcaaatactatcttattagttatattttcattttattttaccatgattttaaatgaattccttaaaaaattaggttagagaactaataaatgataaagagaaaaattattcttcatcctgatagctgttatgtgagggtggaatggtatggctggtttttttcactgtcattttgtaacttggaaattcttattttttaatacaatgacaatattttcatagttaaaaaacactaaaactaaaacaatagtctgttaatgacaaatttaagggcgatttcattaagttaaaaatccatgttaaggcattataagatgaataaatactccaaatgaaaacaaagtgaaggccacttttatttaccttttgtacatgatacttatttttcaacagttgtcatgttagaaaaagttgaaaaaataaaacaagatacacccccttttggcctctaaccttccaaaattgtaatgggtagacttcaaattaaatatctaattcgactcctttccacttctaccctcatagcattagagacatttggaaaaatgaaaaaaatatagtattttatggcagggtacctcaaccccagagccacagacccgtaccaatccgttgcctgttagggacccgattcgcaaagcagtaggtgagcagcagcgggctagggaaggaagcttcatctgtatttacagctgctcctcatcactccatttctgcctgagctccacctcctgtcagatcagcagccacattaaattcccataggagcacaaaccctattgtgaactactcatgcaagggatctaggttgcatgctccttatgagaatctaatgctgcatgatctgtcactgtctcccatcaccccccagatgggaccatctagctgcaggaaaggaagctcaaggctcccactgaatctacattatggtgagttgtataattatttcattatatattataatgtgataataatataaataatgtgtgcaataactgtaatgcacttgaatcatatggaaaccatcctccaacccggtccacggaaaaattgtcttccacaaaatcagttcctggtgccaaaagggtcagggaccactgttttatgggataagtgaaagctggatttgaatccaactctaccttttacttcccatgtgaacattaaaagatgacttaatgtctctgaatctttttttccttctaagtaataactaccttgcagagttattacttgaattggccataaggtaaagaggacaccaaatacagtaaataataacataataataaagaaaccttatgaattctattcagtgatatccatattgaggtatttaaggtgaatgatactgatgtcagcagtttacctggatatgcatcaaaaatatgattgattgatggatggatcggaggaaggaaaagatatgtgatcaaaacgagtatagtaaaatgttcatggtggaatctagatatatagatatcacttgtaatttttaaaaatgttattgcatttttgaaatttgttatgttaaaatattgggtgaaaattctgatgccctggccatgtcccaaatcagttgaatcagatctctggtgtattttttttttaagatccttggatgactccattgtacagcaagtttgcaaaccactattctaacttctcaccacccccttctttatagcttacaaaattatatttttaaatgtcaaatgtcacaatttcactttccttcttcaagaactgattagacttctagcttctcctatgctctcatcttgcataacgttcatttgcacgtggttaaaactgataggcaataaataataacccatctgtcttttcctgacccaacataactaaaaccttcctttcaaggcgaaaatgaactataatctcacagacaagagtagctatagacaactaattctttctggtctctcgtggtcatgggactattcaccactgccgtccctgctgtctatttggtggaccagccagttttactgtgacccataccatggctcttgagaagcatgaagctgccgaaatcgcagttgtagggttaatggaaaacatggtgaagtatagcttacatatatatacaattttggcacaaaatgaagcctaagaaattgtaccgggctgggtgtggtgactcatgcctgtaatcccagcactttgagagaccaaggtgggcggatcacctgaggtcgggagttcgagaccagactgagcaacatggagaaaccctctctacttaaaatacaaaattagccaggcatggtggcgcatgcctgtaaccccagctactcaggaggctaaggcaagagaatctcttccaccctggaggcataggttgtggtgagagaagatcacaccattgcactccagctggggcaacaagagcgaagctccgtctcaataaataaataaataaataaataaataaataaataaataaataaataaaagaaaaagaaattgtaccaggaagtggattgggaatgctctatatattctccccctccaagaagtttttttaatttattgtattgtgaaagatctgcagctatttattttgtgtggtactcttttgcaataaactacttctgcataaatgaactcaaaaactttgatattcatgaaccttcataatgttgatctagtacatcttgcccaactagcccttgggccattccttccatgccactggttgaaaaatatgctccccaccccagtaacatcagcatcagacctctgcaaccagaaagattccataggtaatgaaatccaaatgctggcatttggaagtcaaaccagtgtattttgaaatggaaaggcaaggggaagtgcatggagcaagtacagcatgtgctacaacttcagttcataccctccttaaaatactgtgcaactaaactgtcaattgcccattgatagagatagtatctttttcagctttacaccttccgaattcaacacactgctagcctcaggactaagcgctcaataaatgtttatcaaaagattgagtaaatgaatcagtggtgaaataaaatgtacaaacacatacgcttcatacacgtttaggaagcatttctagtgagctttgtggatattttatttcttaaaggttctttttctttccatgttcatgttattctttattttttaaaatgttgcttctgcttatttctctttctacggcagctatatttacttgcctgagagcacagcatatactgatgttaatcaaactttagatgaaacacctaaaatgatagaaaagaatctgctctaattaaataataaagcaacccctacaggtttagacatgtgccttccggtgtgggaaagaaaaaatttaatgaatgtagtagttttatgccaagaacatttccctcgtgcacctgtgttttaataagatagaatataaaatagcaaaaggggcccgacttttgtgatgatatttactcataaggtagtaagtcagatgtgatgacattttctttgacttagagctgctatacttgggtcagatttcagttcagtaaatttgcagtgaagttgtctttctaacatggtgtcatcctggaactgccctgctcccacagttacatacaggctagggagtgggtaggagtgggggtgaaatcctcttaatgtttatggtgtcagtagattcaaactaaaattagccttacagccatactcctaataaggggcccctggcatatttaattgatttaacaaatttatcaaaaatagataaactgaaatctgccttgaaattaattactgtatctctatttttataagaaaatattttggacccgttccctctgccttatgggtgcaactcccggcagaataggtcatgtctcctgaaaatagttcttattttctttactacttatgacctcttatagcctagaagtttttccttgctatttctagttgaaatgctaatctggcataatttctagttgaaatgctaatctggcataatttcagaactaattttctgttaatgccacttggaacatctaaattcctcctttttcaataatactatatttgtgtgttgcaaacacacacaaatcatactatatttgtgtgtatatatagtaaattccaacttataaaagtaaaattggaatttttttctttctttggaaaattattttataattgagaattataaatgtcacttttttaaatgctgcaacctttgagattggtttcaataaagtaaaacttagtaaacattaagaaaaatgatagcttgatatgttcactaatatggtaaatgaaaacttttatgtgtgtataggctcttgttaattataactaccttcacaggaaaaacagtcttgtggaaggtaatggtgccagtgagagaaaatagagaaagtagaatagtagtaagagagaaaatagaatttcccgtttagaataaagctaagtaaaaagtgaattgtttcagttttatttatgtatttgtaagtgtggtctcatgccatatatattcaaagatagacagagatcttaatctttaattttttatggccaaagaaaatgagtcccatgtaaaagggacattttcgtaagctgatctcaagtggtgtattttgatttgccgcagcagcctatggaagaaatatcattttggctgcctaagaagaaaaatgtcatacctttctattttttattcatactccgcatatttggaattcgtattctcaccatacttttgggaagataacacgaaattgccttacttgcctcgaaatcaaaatctgccattcgttttaaaataaaatggctttttcatctcattttcctttagcagaaaaaagtaagttaaatttttttgtagctgtgttaagtgtactgttcctccctcgttgagaacgaaacaaagacatatgattccatttactgtaattgttttggtttgcccaattacaatagcagtaaaatcatttacacatagtaaaatgtttgagccttaaagggcaagaagactaaatttagctgaagatatacaactttcatgtgccaatgaggaaatgtataaaatcagttatacatattttcctcattcatagatattgaaaatattcatatatgtgtacatgtatttgataacttaatattttctaaataacagaaattatatactgtatatatatttggatattatgattattgttattgccaccattgagcacttactatgggctcagaaaacttcactgtattatttttaattcttataacaaccctgtgaaatatatattatgcctatttcacaaatgagaactctgaaatcaggaaattaaatcacttgctgaagtttacaaagcactgttaggcaataaaacagaatttcaaactcacgtttgcttgtcttcaaattttgtatgctgtacaacctgattgttttactacttgttcttaatttcaaaaaaaatcttcatactaaaagatgatactttgggagttctaaagaacatgtttttggccgggcgcggtggctcacgcctgtaatcccagcactttgggaggccgaggcagaaggatcacgaggtcaggagatcgagaccgtcctggccaacaaggtgaaaccccgtctctactaaaaaatacaaaaaattagccgggcgtggtggcgggcgcctgtagtcccagctactcgggaagctgaggcgggagaaaggcgtgaacctgggaggcagagcttgtagtgagccgagatcgcgccgctgcactccagcctggttgacagagcgagactccgtctcaaaaataacaaaacaaaacaaaacaaaacaaaaaaaaccatgtttcttttcagaaggtatactcatttgaagtggataccaattatttgtattaaaattacttatggataaatttgaatctgcaaaaattaagtgcaacattattttttggcacctgttataggaggtaaaaatggtgattcaatatagtttctacccttagggagcatttagtgtggtttagacgtaatgacactaatgtaaattagagtagaataaattctttaaagcaggacaaaaggagtgttaaagactacaaagtttcagttatggaagataaaattctggagatctattatacagcatagtgcctatagctaataatgctgtttcggacacttaaaatttcctaagaaatcttatgttgtgttcttaccaccagtaacaacaataataaaggggacaagaggaaattttgagtgatgacagatatgtttatagccttcatggtggcgatggttttgtgggtgtatacttattcccaaactctttgattttatatatatatatatatatatatatatatatatatatatatatatgatgaaactctttgagatatatatatatatatgtaaaatatgtacagctttttgtatgtcaatcatacttcattaaggtggttacaaaaaagaatcaagaaaaataatcaccctaataaaaagagtgttattaaagaggggagggattacttctaattggtagtatcagaaaggttctcatggaggaaggtatagaaaaatttaggacaatataagcaacgatactttaagaaaaggaataatataaacaaaaataaaccaaaagatgaagaaccaccaaggatgtttctgaaaaatcaagagaaattccattttgatttgagagtatgaggtacttgtcatgatatataatgagaagtgtctagaaatatgttattttgagcaaatgctgcttagaaaggaggttaggttatttattaatccattcctttgaaatataaatttcatgatcagaaacacgtgagtgtaactatccaataacaacatgatttatttccaagaggatagtatattataggagaagtaggattttttctctttcacaagctaatacagtgtttactgattcaaatgacacttaaaaaaaacaagaaacaaaaaacatacactataagctatccttattcagctatgttattgtcttgcattcctgattagaaacatacaagttagactcccaaagtctcagttttctcatttgcaaaatgaataaaatgatagtacctaccccaaggtgctgtggagtatatttaatcatgtacttaatctgaagttttaattagagtgcatagcatatatttgtggttgcttttgctcctatttctgggtaaaagttcctacacctattatatatgttttgtgtttgaatgaagagaaaggcatcattatttcaaatatcccctctagccaacaaatgaaagctaagacttggaagctaggatgtctaacaccatatcctcttcatgtggactatatagtagaggatatgtaaattaagactcaattaaatgctcacattttataaaggagacccttgaatatatcaaatcctcagagagtatgtcagtttaaacttattaagaaatttaaattatttaacttagcctgctgagtcagtttctgtagctgatttctccaaaaggtcttggtattaaaagtagcattacagatcactaatagcaaactatttctgctactttgggtatttttacagggtgctaattgtgccaaatcaagacattgaaattgcataattttaggttaatattagcataatcctacatattttttcagaagcagtgcaggaatagtacaccctgaatatttttccatcatgatggaattttcaaatatcacaacctttacattgtcagttgtctgctggaacattatattgtgtcaaagagatttataaaagagaagggcaaagaaaatttgcggagttctagacaatagtaatatcagggacacttttgctttctttgttattactaaggagttaaataatcagataccaagcataggtaaagaaatgaatctctaactatttgagtacttatcaatttatctcaaggatatttacggtgttttcccctaagccaaaatagagtgcttaattaaggtccccttagccccctaaaaatcactcaggccaagtttctgatatgtgtgtatacttatatacatattaaagtatatgtatttttgtgtgcactttcagatcaaaaagtcctcgtttatctaaagcaatttcagcttattgatgaggcttttctgttacagcgtcctcctccacagatctgcctgtctagagatttaagcccaataagagtccagcaaagtttaaacaacactttagaaatctaaagaattttaagatttattttaatagtcttagtgataacctgaattgtttgtgagttcttacaaataatgcaatcaacatttaagtaattttattatttttttgtttgtataaatttaaggtatacaagtacatttttgttacatggatatattgcagagtggcgaagtctggttttttgtgtacccatcatgcaaatgatgtaccttttctccattaagcaatttcctatccttcgcccccatcccaccctctcgcccttctgagtctccagtgtctattattccacactctgtgcgcatgtgtacacattatttagcttccacttgtaagtgagaacatgcaatatttgactttctgtttttgagttattccacttaagatgaccaccagttccatccatgttgctgcaaaagacatgatttcattctttactatggctttgtagtatttttcattgtgtatatgaaattgtttattccatacgcaatttgtgtgtgtgtacatatatatatatatatatatatatatatatatatatatatatatatatgcttagacttagaagctaggatagacacacaatggaatactacacaatggaatacattcattcacacacatataaataaaagaatatgtggagatatatctccacatattctttatccaatcatctgtttttaaataatgctattgacttctttagggtgaattttatcaatattgttttggtttaaaacactcaccttaaaagagtcacagtccctaaatgtgcatcctcatatttaaattaggtctcagtaaatttgtgcaaagtgtattctttttaggatggtgttgaacttgctaaattatttatctttaagaatcatcattttgtgtcttttattaatgaaaacaacaattatgtgattgctgatatatttggaaaatgatttctgatgtagattgatttttttattctaaattctgtgtcggtattaaaaatttatagattactaactgtattaatatcgataatactaaattttattgctatttataacttggagtgtactttcatcctcctgaaaaagctgaatgaggtaggcagtattattctgggtttatgtgtgagataactgagactcagaggtaaaatagtgtatccaagcattcatggctcttaaatggaagatataaggggtttgtgaaattactcatggacttttttattcattcattcagttattaaaatgtattcaacatttatcatgtaccaggaacagcgcttagtaccaggaattcaaaggtgcataaaacatcttccttattctaagaggtacatagtgtactggaacaaacagccttgtaaatacataattagaacatgaagtagtatgttaatagaggttttcacaaagctgtggaagcttgtcttatgaagtaactaattccaagggagagaagccttatggaatagtgacattttagatagggtgtcattctaaaatacagcaaaaggcccacagtaaaaaaggaattttggttgttatgaaaattttcagattttctatgttttcagtacagtatacatggtgggctatgtgaatgtttgtatagggaccaaagtaggaagtgaggttgtctgttagagagcgctgagaaaccgaaaatagggagagatgagttggaatatgctgaggaaaagttattaggagttttcaagaaaggccacgacagtggggctagagagaagaggctaaattaaagagtcatttctggtttagaattgataaaatatagagacaagcatgataagaaagaagtcgagaagtaaacgatggtctcaagatttctagcttggaaatcattgactaaaattaaaactaaggactggattaggccattcttgcattgctataaagaaatacctgagactgggtgtttataaagtaaagaggtttaattggctgacgattctgcaggctctacaggaagcatagcaacatctgtttctggggaggcctcagggagcttttactcatggtggaaggcagagcaggtgtaggcatttcacatggcgaaagcagagagagagagttggtggtgggggtgggtggctacctacttttaaacaaccagatcttggagaactcactcattttcatgaggacagtaccaagaggatggtattaaaccgtgagaaaccaccctgatgatccagtcacctctcaccaggccccacctccaacattggggattacaatttaatatgagatttgggtggggacacagatccaaatcatatcaaagacttgcatgggaaaataaggaattgttgacataacatctttgaggttcacatcaaatgttctgatgaggatagtccaagtagcagttggctatatacctcagataagggctgaaatttggagctatgtcataatcagcctagattaagagtcaataatctcctgcccatgggccaattacacccaccacttgtttttgtaaagtagtattgaatcccagccatatccatttgcttatgctccatgtataccttttttttgaacttcaaggcagagttgagtagttgtaacaaaaaccatacggcccacaaagcctgaaatatttgttctcaagatctttatctataaagtttgccaatacctgctgtagatgttagttgaagctttgaaagcaaatgaggtttcataaggcagtgtccatacaagacatttaacaagtttacctataaaaactagaattcctttgaggggaacacatcctagtctccattaagcacagtagaagagtcccctataatgggaaagaggtcactttaggtgttgatgttggtggtacaggtcaaagaaaatttatctttgctgtttattcagaatgcaataagtgaagttatgagaaataagggaaaaaatgtgtagaatttcaacagcgaagagaggggataaaggcatgagaatgagttcctaagctcaagtattataaacactgtgagaaacttaaaatcaaagtatgactccaaacgtatttgaagcctgagaacaaggctcacaacctagggaggattagggatcaataaaatagagtgttacaaagtataatgtcaatccagagttgtaaaaatatcagcattgaatatattgaaagcagtaaaactgaatgaggagactatcattttatatcactgtgtttatttctttgccttgttctataaatatttaaaattataaaatttttattaacagtgagagcagaactaccagagtgagcagatcaaaattgggacagatgcttttcactgcacacacttttatttttctgctgttcatgcattatcttgtacagtgcacatgttttacctaaaaaattaaaatggagtctcctgcttaggaaaaaagtatatattctgtttcaaactatatacaaaaataaaatcccaggtgactaaaaactgacatgagaaaaaaacaaattgataaagcttttacagtaaaatagaggagaatatgttaattaatatagggtaagaaaaaattgcttacacaaatgatgaagcactaatcatgaataaaaataataaagtggactaccttgtatattaataacatctatacatcaaaagacagcactgagagagtaaaaatgaaacccacagagtaggataaattatttggaatacacacataatggatgaaatgtgtgtattcataattataaagaattcctacaaatctttcagaaaagaacagataatccaatagaaaaatgggaaaagttcttgaaaagtgaaccatggcacaaaaagggcttgtggcctgctggcaatattctgtatcttgacctggatggcatttttaaggtgatcactttatagtaaataactaatgtgttttatgcatcatagtaacgttaagatttttgtcatctttacaaaataagaaatccaaacggccaataaatatataaagaatttctaagtcccattaatggtccaggccatgcaaattaaaactaaaatgaaatatcactgcttaccaaccagaatcattgaaatttataagtctgacaattccatgtggtggtgagaatatacagcaattagaaatttcacacaatgttacttggtctgtgaattgtaaatagaagtgtaaaattacactactgcttcttggagtgaaatccatttggcactatttagtaaattcaaagatctgcataacctatagcccaccaatttcacttctatatatacactctacagaaatgcatatgttcatattccaggagacatgtttgggaatgtcatagcagcatagtaatagccccaaaccaaaactacttcagtatttattaatagtaaaatttgctatagtttgaatgtgtctctttccaaattcaggtgtcgataatgtgctagtactaagaggtagggtgtttaagtggtgattaggccatgagggctccttctttgttaataaaaataagacccttataaacaaggcttcacgcagcattcagtcagcttgctctcttgcccttctaccttctgccttgtgaagatacagcaggaaggccctcaccagacaccaaatgccagagcctttatcttggacttcccagcctccagaactgtgagtgaatacattggtattatttgtaaattacccagtctcaggcattttgttataacagcacaaacagactaagacaatcatacagtgagaaattaatcaacaactaataagcaaagaggtagattaatcttgaaactatgatatagagtgttccatttggctgctggaagttttatttcttggtctgggtgatggtcaccatgggtttatatgaatggttccctatattatgtttcacaacaaaaagcatttaaaaagtaaatatatgtaatgtactcagggataggcatggccaaccatggattctatgctgaaataatgattcagatttcatcagcaggctaatgacactgcctatttaaatactttaagtcctgaaattaaagaaggtaatttctcaagaaggaatttctaatttatgggtgggtctattccccaccagagagacactagcatggctcagattctatgttggtcattttatttgcatttaaagtcttaagccaaatagaggtacactaataatgacaacaactactactactcatacttgtggaacactgccagatgctgttttaagaaatttgcattttcatttgtaactgagcttacttgaatcttctctctttttttcttggttaatctaactactggtctatcaattttacttatcttttcaaagaatcaacattttgtttcattgatcttttatatttttgtttcaatttcatttagttctgctctgatctttgttatttcttttcttctggagctttgtgttggctttgttgttgattctctagttccttcaggtgtgatgttaggtagtcagactgtgaactttcaggctctttgatgtaggcatttggtgctagaaaatttcctcttagccttgcttttgctgtatcccagaggttttgaatagattttgttgtgaatgtgatgaaaacggaacatttgtacactgctggtgattgtaaattagtacaacctacatggaaaacagtatgaagatttcttaaagaactaaaagtagatctaacatttgatctggaaatctcactaccgattatgtacctagaggaagagaattcattatatcaaaaagacacttgcacgcatatgtttatagcagcacaattcacagttgcaaagatatggaaccatcctaagtgccagccgaccaatgagtggataaagaaaatgtggcatatattttcatataccgtgaaatactattcagccacataccatgcaatactactcagccgtagaaaataatgaaataatgtcttttgcagcaactttgatggagctggatgccattattctaagtgaagtaattcaggaatggaaaaccaaatactgtatgttctcacttataagtgggagctacgctgtaggtacacaaaggcagacagagtggtagaatggactttgaagactcagaaggggcagagtgggaaggtagtgagggataaaaaattacctttggggtgtaatgtacactacttgggtgacacgtgcactaaaatatctgattttacttctatacaattcattcatgtaaccaaaaatcacttgtattccaaagactattgaatttgaattttttaaaaacattaataaaataaaagatgtaaaaaaagaaatttatatatactcatttattgagctcccacaattaaccttaggaggtaagtacttcataattggtagtatacttatcttttactaaatatttgtattacttgggaagttgagggttggggagaagtagcaaggtactatgatttggggcagataactaacttatttattcgcacatacagtttggaccatgagacacgagctcaggtccctcctcctcacctaatcaaagatgaaatatgtgggatgggatgaaataatcagcagtccaatgctgagtttccagaccgaagtataaagcaacaatggatatgtcagaagtctactagggtgttatttatttaaatctatttcatggaatttactaccaccttaatggcccgaaagtgttaaagtatgccccagagtaccgaattactccctaaatgtaatttatgcttgagaataatctgactaacttgatttagaacatcagaaaataagttatgctgcacataaatgaagcagcagtgtaattttaaataccggttgcacggtgaatgagaattttaatatttgcaaaattctaaaatcacttgatttattatccttatgtttatactgacatttttttgccctttgttaagttccatccatatttcttcttactgccaagaaaaaaaactttttttcctagaaatattacagaaggcaaaaattatatttgtttccctgaatgctatttttgatgtctctacttgtttctcattgttaccatttgcttcattcatgggcagcccaattaatggagcgagacaaatttagggagcacagtgactaattagatattaaattggtaaatctaactttgtaaaaccagaaaaaatatatatatatttttttcatttggaattttccttggtggaaaagagtttaaaagtagtcatgataaaaaatgtaattttacgtagtaaattcaagaatagatttagactgtgctattaacagcacctattaaatactgaaaagtgtattttaaaattttatgtgaggcttgaaatggagtctaaagtattattactcacattaagtgtcatcacatgtaaagcccatgattttattctttaatattttgtttgaatagttacttatttcaacagtaatttcaataataaaattaaatcaactttacagttttcaaaggtttagcagttgcatgctgtaataaatacttcatatttatatatttataaagtgacagcataagtcatttttattaggtccttgaggatgcaaaagtttggattatacgaggagacgagagaaaaagggaagaagggcatttcagaaatatgctaccgatatgcaaattcacaagtcctaagacagtagcaggggtcgggcagaaagtccatcctgcctccctcttgtgggcctggaacaatggtgtaagtggaaggcctgttccccttctcttcctacctccagctctgtcttacagagctacggataccatgagcaagtgtatgaacccttacggttttcttctcttgggagaatgtaaaggaaagataacttgtagaaacttgtagataacttgtaaaaaggaaaagaattcagggtgagagggggatttgttgaatttgatagaggatggcaattaccaatatgatgagtgattgagaaacaagtctgtgcaacaggtttgaaatcgaaaatctttgaggtgtacaggatcctgaaatgaagaatgggcatttatagcagtatgtcagagaaacagtcacctcctagtagctaaaagtgttggcaaaagtatagttcaagtgattgggtaggaaaaacagcaaaccaagagtggagactgatggttgctacaaaggtggagtggtaagtcgtgaccaactggtacttctctgtgctctggttagctgctgactgtttctcagactgtggtagcaggaggagggttggagttagcagtcatttgcatatgagactgccatttaaaaaaaaattttaaattatttcatttttctgactctcaatatgaaaagcacattgtagacaaattgaaaaatatagaaaaattatataagaaaatatagtctcaccagtatggaacaatgctaactatgttgcatagatttttagattctcattcaaaagcaactctttgactccagtgatgcaaatgcatgtaacatatgcaatgtgcaattcatttttaaagggaataaacttacgatatattcataggtcatttattgtgtgttatataccattgaaaatatatgaatgctaaattattagtaaacatgcaaaaacattggcaagatcattttgttgtggaaggatatattgtatctgaataactctagaataccataaatcatcaaaggcaacattcttatttttcactaactacagttagagaatacctcttcggctaccttcggttgccttttttatgctaccaaaatgctgtctgttttacaagattttaaaggttaagcatataattattcattaaatacaatgagtgcaatgtacatgtagatacattattaaattttgggtagttaataaaaataaggggaaaaaacctctagaactatcacttttaattgtttaactgataaagtgaagcttcatcttggaaaaataatttcacaagagagcatgtgcactggtagaaaagtgccattgaaacaagagatatttgggttagaagcctctctctactatttaataccattttcaccttttggcaaattacttggcctctgttttctccaatggaaaatgggaataataattgttatgctgcagggttattgtaggtgtcaatgaaatgatgtgtctggcactataaaagcacagagcccggtgcctggctattagtaactgtttaataaatgttaattcctttctctgcccaggacatcagtaggcagatgtagcaatttaaaacttctagtgttactttaaattcctgaatgaaggtagaggactgaaaagatatcatggtattcaaaagtatgatccattgcttcttaagaatagagttcagaaaagcttgacagattcctgtactctgaggcagcaccatagccggtaatctgtaggatggctattggttttgtgctcacaaatgcttgcttgggcaggccccaggaaatctggtagactgtaagcccagtaagatttcaaatcttactttacggcagtgtttttcaccttgactgtacattgaaatcacctggatgctttgaaaaataacagcgtcagtgtccaacctccagaaatactgattaagttggtctggaatggagccccaggatcactgtttggttattgttgttgctgtgttttaaatgccccagttgattcttatgtgcaactgtcttaggtaaacatacagccctggttcatattatttctgcctcagtctcttttatgactggaaggtgaccaaatgcttgtttcctaatattctttccatgtgtagtattaacacatttgacttgtactaagttcctgcagtattccaatctaaaattttagtgactacaataaaataagaaggattaaagaaggcatcgcatagtttagtatatcggttatttaatgcttacatgtgagcctacaatatgaattatatctgtcatcttattttaaatattgacagaatctttaatgatagtgacgaattattgatttattggtgtgataatggtattttagttatatttttaaagttttatttgtaataactatatgtatttatggggtacagtgtgacgtttcagtgtaatgtttcattgtgtaatgatcaaatcaggtttcttggcagatccatagcctcaaacatttataatttctctgtggtgagaaaatttaaaattctctttcactattttgaaatatacagcacaatattggtaactttgttcatattactatgcaatagaacactagaacttattactcctttcagttgatgaacaggcagttttggatcaagaataatattgaaagtgatagaatttatgaagtaatttttatccaaaaatattttgaaagggaatatattgcttccaaataatttattacaatgttaagatatttgtaaatttctagaattaaaaaaatatatttttaggaaagaaaatgccaatagtccaaaatagttgctttatctttcttttaatcaataaatatattcattttaaagggaaaaattgcaaccttccatttaaaatcagcttttatattgagtatttttttaaaatgttgtgtgtacatgctaggtgtgtatattaatttttatttgttacttgaaactaaactctgcaaatgcaggaaactatcagagtgatatctttgtcagtataaccaaaaaatatacgctatatctctataatctgttttacataatccatctatttttcttgatccatatgcttttacctgcag

SEQ ID NO: 270 - DNA, 50 bp, Homo sapiens
  source 1..50: mol_type = genomic DNA
gtaggtaacacatatatttttcttgatacttgcagaaatgatttgttttc

SEQ ID NO: 271 - DNA, 250 bp, Homo sapiens
  source 1..250: mol_type = genomic DNA
ttttaaagggaaaaattgcaaccttccatttaaaatcagcttttatattgagtatttttttaaaatgttgtgtgtacatgctaggtgtgtatattaatttttatttgttacttgaaactaaactctgcaaatgcaggaaactatcagagtgatatctttgtcagtataaccaaaaaatatacgctatatctctataatctgttttacataatccatctatttttcttgatccatatgcttttacctgcag

SEQ ID NO: 272 - DNA, 60 bp, Homo sapiens
  source 1..60: mol_type = genomic DNA
ttttcttgatccatatgcttttacctgcaggcgatttgacagatctgttgagaaatggcg

SEQ ID NO: 273 - DNA, 148 bp, Homo sapiens
  source 1..148: mol_type = genomic DNA
gcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaag

SEQ ID NO: 274 - DNA, 55 bp, Homo sapiens
  source 1..55: mol_type = genomic DNA
gcgatttgacagatctgttgagaaatggcggcgttttcattatgatataaagata

SEQ ID NO: 275 - DNA, 49 bp, Homo sapiens
  source 1..49: mol_type = genomic DNA
aaagatatttaatcagtggctaacagaagctgaacagtttctcagaaag

SEQ ID NO: 276 - DNA, 60 bp, Homo sapiens
  source 1..60: mol_type = genomic DNA
gaacatgctaaatacaaatggtatcttaaggtaagtctttgatttgttttttcgaaattg

SEQ ID NO: 277 - DNA, 248401 bp, Homo sapiens
  source 1..248401: mol_type = genomic DNA
gtaagtctttgatttgttttttcgaaattgtatttatcttcagcacatctggactctttaacttcttaaagatcaggttctgaagggtgatggaaattacttttgactgttgttgtcatcattatattactagaaagaaaattatcataatgataatattagagcacggtgctatggactttttgtgtcaggatgagagagtttgcctggacggagctggtttatctgataaactgcaaaatataattgaatctgtgacagagggaagcatcgtaacagcaaggtgttttgtggctttggggcagtgtgtatttcggctttatgttggaacctttccagaaggagaacttgtggcatacttagctaaaatgaagttgctagaaatatccatcatgataaaattacagttctgttttcctaaagacaattttgtagtgctgtagcaatatttctatatattctattgacaaaatgccttctgaaatagtccagaggccaaaacaatgcagagttaattgttggtacttattgacattttatggtttatgttaatagggaaacagcatatggatgataaccagtgtgtagtttaatttcaacttgtggtgtcctttgaatatgcaggtaaagatagattagattgtccaggatataatttggttgctaaattacatagtttaggcataagaaacactgtgtttattacacgaagacttaattatttttgcatcttttttagctcaaattgttcatgttgcaatagtcaatcaagtggatttgaattgtagccaatttttaatgccagaaaatactgattaagacagatgagggcaaaaaacacccagtagtttattaaatactttagatatttcaaaatgctggattcacaaaagcagtatcacatttgactttacaagtcttcattctcaaatatgtttccatagtaaatatgccctttaatattaaggagttaagcatttaaacacctatttatatgataagctatttaaacacagaaaatatttttaaaaccttgtgtaattatatgtgtatcaatcaaacttgcatgcacaccagcgttggcatttgtatagagaggaaatgtatggattcccaatctgctttaatatagaagatacattttaaaaatagcactgaagtgaattttgggctaatgtagcataatggggtttctgcctgagaggcagaaacatattagagttatataaaatgttttggggtagatatagaaaccacttgccattttcaatgatatccaacccaaggtagttatatatttcaatttatattttattatcaaattagtacttattgtgaaaaaaatcaagtaacatagaaatttgtaaaagtacctccattctactctttggaggatagttgttcagtatgaattttgctacatatttcaggctgggtttcttggaaagccattgtaaaatggagatttgtatgtagaaggttaactagggagtacttttacgatgaagcaatttgttttgatgtaacttggtgtagttttcttcatgtttcttgttcttgaagtcagttaagctcttgaatctgtgcatttaacatttcatcaaatttagaaacctttcaaccatttttttaaaaaaaatggaactccaattgtacatttattaggctccttaaagtgccccactactcactgatgttatgttcattgtctgtttggtctctcttttctctgtaatttgttttatataatctctattgtcaaattgactaatctttttcaaagtctaatctatggctaatcccatgtagtatatatttttaacatcagacattttcatctcttagaagtaaaagttgggtctttttatttcttccatgtgtctactcaacatgttcagtctttactttcttgactatatggaatacagatataataactgttagaatattcttctctactaattttatcatctgtgtctattctgggttaatttaaattgatttatttttctcctcattaagtgtgttgtttaactgcttctttggatgactggtaatttttgactatatgccagacattgtgaattttaacttagcgcgtgcttgatacttcaaataaattcaaatatattgaaataaatattctcaaacctcgttctggaacacagttaattcacttggaaacaatttgatcttttgagaatcttccttttatgctttgttatgaccagaacagtgtaagtttagggctactttttccccactactgaggcaaaacccttctgagtactctctctgatgtcctgtgaatgataaaatttttcactggggctcgtgggaacaggtggtattactagccacgtgtgagctctggtgattgtttcctttaattcttttgtgaagttctttccttagctttgagtggttttcttgcatacatgaactgatcaagactcagatgaagaataaaataaagctttctacaaatctccaaaatttcctctgtgtatatatcacctctctggtattttgccctgtgatcactagtcagccttgggctgctgaaactctcagcttcatcttttaacaaaagcctcctggcaaggatcactgtccttcaatgtctgatgttcaatgtgttgaaaaccgttgtagcatatattttgtctttttttttttttttttttttttaagtgtttcaggtgtttcaggcaggagattaagttcagcctcctttactccaacttgaaaacaagtccaaaacaaactattttgatgtaatttgatcttttaatacattaacattacacaattttgtgaatatatcataatttaaaattttcagagaatgtctaatggtcctcatttcttgacagtgtggtttagttgaaactgatgaacattttatcaaaacttttcccctcaattggatacttttttttttttgagatggaattttgcttttgtcacccaggctggagtggcatgatctcagctcactgcaacctctgcctccaggcttcaagcaattctcctgccttagcctcccgagtagctgggattacaggtgcccacccccacacctggctaatttttgtatttttagtagagacgagatttcaccatgttggtcaggctggtctagatctccgacctcaggtggtctgcctgtctcagcctcccaaagtgctgggattgcagacgtgagccaccatgcctggccaactggataattttaaaaagaccattttatttagtctattttttctcaatctatagatgagataagaaaaatcattctagatgtccaaggaaaaattctttcagaaaagagctgtgaatgatatcacaaaccccccaaacagttaaggtatttctttcctggttattttatgtccaaaatcatgcatatgaacatgtgcacacacatgagcgtgcacacacacatgaatacatatacacgcacataatgtaccttaggttatctttccattctgagtaattatcgtaaaatgggtaaaatcaaccccgtaagataccttcatcgataaggcaaatcaaagctttggtaatttctgctatcttggcctttgttgattgactaataatgaataagagaatgagtttcaatatttactatgaaattattttagaagacaggatgtagacagtggctgttagcaggcaattgtttggcatgagccagtaatggttactgtgaaaaaaatcaaccaagcagcccatatattaaacaaacacacgcagaagcacgttggagtctgaagcctcatatgtacaattttcagtaaagaaataacttttagatatgaaataaacaaatagatatatgttgtaaacttgtccctatgtattttgatcaaattgcatcatatttttttcactttaaagaagagaatttagtgctttaactgagacttagtgttatcattcaaaatatactgactgccaatagcagtagaaagataatctggttccatgcaactctattttttttcctctgtcgcaagtaaaagacaaaattaagtacatgaattagtgctttttgaagatattccagagcaatataccatgccactatggagaacctctctaaaaatatcccatttttttacctgagaaaaatattgatcatgttatatgccactcaaattggtttattaaattcgttgaatgatatcagcatctcttaatgcattcactaaacaagcagtaattgagtgcatatacaaagttttatcatccaccaaaacagtgacaatccacatgaggctctaatagaagtttagaaagggggttaagtggttaaatgctggactcagaaagattggattcaaatcccaggtcctttagcttaatagttgtagaatcttgtgaaaatatcttaattcttttcatgtctctgatttctcttctctaaaatggaaatataaatgagatgtgtataaagccacttggaatagcattttgcacaaaataattactcattaaatgtaagcccctattataactaatcactctttataagtgattagttcatatcaatacaaactaagacttatttactgaattatcgtctctaaacatccacactgcagaaaaaccaacctggaaatttcataaaaccttatttttatgtagtataatttcttctcaaagcataagggctcttggattaggaattgaggaaaattccaattcagccaaacgcatctgtttcagatagctgacacttctgcctactcatttcctagctaacaagaagaaatgttaatgggagttttcaaaggaaaagctgaacaccatgaaggaaagtgacacaaataatgttagctcatatattgacagggtgaatttgtgtgctttcaagtcccttcagtgaaaataggaaagtagaaattataaaatgccctaacatttaaagctagcatgttcttggagactaggaaaaaataagttttaaaacatgggctatgatagaatgagatggaaaatgtttgtagttgccagtagaaacaataacaattaccattagattaagtatttaaaccagctgaatatttttattaatggaaatggcatctgttttatgaaataatgctgctgaatgaaccatattaaaaatgaccagtatttcctgcagaacgttgtcgcagacatacaagcctgagaccctaaaatcttaaggtattccatttgaaatcgaccttaagacattaacagtagtggtattgtttagatgaaattttttaggctttaaatcaacaaatgttaagcagacatggggagcgaaacaccagtgtgttattctgacatgaataaactgctgtttttagggaaaaaatatagtcttgttaaggttaagctaattggttttctggtatcttttgcaatgttagtgtgttttactgctccataacctatgttatatggtaaatgtgcaatatatttatatatgttgctgtaaagaaatgtaataaaaaactgtttactttgtgatatgaaagtaaaaatttattcattgtcattgagcatacagaagtaaatatggattacatatgtcatattttaatgttcacatggtcccaccatcaaatgttgaaaaacttatagtttaacgtcatattctattgaagaaaaatacactcccttttctcaaatgtgaaatgtccagagagaatggaaaattacatataaagcatgtagttatagcatggtgaccctgctgtgatctctcagatgaggaacaaaagggagaaagaaagagcacactggtgctttggagttgagagaaggcaaaaaaagagtacaaaaatgtcaaagccaagtttagctgctcttcagctctccctttagctgctcttcagctttaccttaccatggttattagtgattgaagaaaattctaaagcactttttaaaggacccaattctgaagagtttagattcagagagcacaatggagttggagtgactcctgctcaaaagtttgagacaagcgagtccatgaaaagaccgtcctcctcttaatggaaatacccaggttttctcattcttctcgccttgctttcagcactcgcagcccagaaagcccttatctaacaggtactgccgttgaaaggtcattgacttgtacaaaaatgatgagtgctgaatagatgtgcataggtcactgacagtatctgctacagagaatgagttttcgtatttttattaggatacacctaacatggcaatctactgcctcaaagaactctataggaggtaagtgaatttatattaatacagattgaattaaaggataatctagaaaaaggcatatgatgtaaaaaaatcagacacaagtatattttctgtatagtcagtttttacattgtgatttcaccagctggctgctgagtttgacggcttcttaacagccacactgctgagattcaaatgctgatagaaactttgatggaaaaatcactggagtaaatatttctaccatctgttgcccttcactgggaccctaacgttaagaataattcataccattgcttgtcctttatatttccccagcagtaataaaatttcataagattttgttttgtggtcacaaagctatcctggtttctgtaactagaagacatacactagcataagggaatcagccggaaaatttactgctaagagaatttgtctctagtcacttactttaaggttacagcaatgtgtaagtgtgggaatacattttaaaatgagcttttcaaagttattagctggtagtggcatgagagttaagtctcttaatacagttaaacagttgggcacttcatccttgcgtaaatattgttacccttttattgctgcttggaaactcctctgcaactttttggcccctatccatcttttcagaagtagtaaataaccaatttactgggagtgtggtaccaggcagaaattccgagaggggctttcaatccttgcccatcaagtgtatctttcagaaataagtatattaaaataattggataatttcagtggcttgttattagacttccgttgtccagcatggcatgtttaagaagatgacagattttcatacattattggaaagaagcaagaacaaaaaaacataacttactgtagtaaccacggtaaagaactgcttaaaatgcaggataaacatgtcatccctaagggattcccattcttagagcatgaaattatcaagagagtaagagactacaaaaaatgagaagaatgctgattgcaaattccaaatagaaaaaatcaaaacaaaactgcgcaccatcattctggaagcaatgagaagcagaaattgtcatttaatgaaatgtaagattaaagttaatagaagtaattttcatgaaataatattttgcaaggacgatgttccagccatattgatcttcgtgttttcttttcacatcccttcttactgttccctagaatgcttgtttctacctttaaatttgcttttctctctaccagagggctctaccctatctccagtttctcaccatgtcccaatctactccctctcagaatttttgtacacttccctttatatatatttgtgctctaattttatattcacagatatgccttttgtaactcccccatcttaaagaaagcacacacgtacgcacacatgcacacacacaaaattgaactctttctgggagatctgcttaactttcttcataactctgtcacttgctgaaactgtagtatgtgttttcatgtttattatcttttccattagaatgaacatattttgggtacttggtctttctcgatcaccaatatacctcggtacgtagaaaaattgattcatatattgaaaatgtaatattcagtagaacgaataaatacataaataaatttaaaaatgatacttttattgtattacctgagacaaatgatccccaagtttgtccttgcttttcatagccaaaacattctctcttacattgagcttccttcacctcttctgtgtacagagcacttaaaattttcacattgcctgatactttaacaatatgatggccctgttctcttacccattggagcatatgttaaataccagaacccatgtaacaaacatatattgtgatcctactgtgtgcaaagcagatactgcttgctgctaggaatacagagctgactaagagctccttttctctttatgagctcacagtctcatgagttcaacgtcttaaggcacaacgtctaaagcaaagggcagtaagtaaacactccagaaagtactggatctggcctaggacaaatggtgggttgtttttccagctgttatttttcctgccccctaattgacagtcctccattacacctctgggatacctagtctgacttgggaaaacctgactttgggaatcagaggcagtctctcttgcttatatatgaggaactctaatggatacttactgtcattagagaaactctgcttctagcctggctccttttgtaaagaaggttgagtccccttggagagcctgcagaacataaccatttgcatgtaatgaacagtttgtaatactttgagattgatgtgcaatttctatttgacaagggaaaaacaattaggattaaccgtggtcgtatatcccagaataccaacgttgtttccacactctaagtgttgttgggtcattatatgagattcataattttgtcctgttgtacccacgtttgcattaccattcagtcttaatttattataccctattaaaagtttttttggtaatttgttcttattgctactcaggcattaaaatgtctgcaggctgtgaaaatgaataaatttaatgtggcagcatagttctcaaaatcctggctttacaactcatagtacaggcttgtattgtaaatcctagttaacatggatttatttgaaaatccaattttactgctaatcttaaataacacatttttcaaacattttatccttgaatttctatttttttataatttatggctgttgtatgtatttacaaaaggacaatgtgtgtacttttaaatactagtaatggattgctgaaacaactgtaactttaaaacaatgcaattgttaaaaaaataaactgtgcagcctggcttaatggaggcttatgaacatatgattaagatatatgctataataagcaaattcactcaactgatagttcataggaactttcaaatttaatctcataaccagtgctatccttcaaagaatggtcagggcaatttaacgagtacatgaccacgcaagataatttcattgaagagtggctgaactgttgaaatattttctagtctccttgggatatcattaagagcagaaattttgaaatggaattgtaatgatgttcagaaaagataagtaggtaactctcttaatacgttttgtgctgctgtaacaaagtacctaagactaggtaataatttgtaatgaacaaaaatgtattggctcacagttctggagactaggaagtctaacattaaggtgtcagcctctggcgagggcctacttgatatgtcatcacatgatggacgattagagggcaagaaagatcaaaagggggctgaactcccacttttataagggaaccaaacccactcgtgagggtggagccctcaatccttaatcacctcctaaagctcccaccccttaatactgtcacaatggcaattaaatttcaacatcagttttggagggaaaaacattgaaaccatagtagtgatactgactactaccacacagggcttgggaggctaccctagctgttgcacccaagagatgaatcttctaatgtgattacctttatcattttttttactttattaaaatacttttattttacatgtatacttttgtctacccaccatttccatgtctgaccactgctactactatgtcctagcataacattccatacatccttaaaaccaagcaaagggtggagttccatctttaaaaactaaacaggcattttggacaacacattcttggcaatggaatctggacaacatttatcaaacatggtagggaaggttctcactctgcattatcaaaacgacagccagatatcaactgttacagaaacgaaatcagatggaaaatttttaacaaattgtttaaactattttcttagagagacttcctccactgccagagatcttgaatagcctctggtcagtcatctggaagcaattcttcacataattcatgaacttggcttccactttaggaagagaaccacctttttctatacttgcttgcatttttgctttaatgtcttctacagaactaggtcctttgggtgttttaggagtttttccttgttttgaaggattcttgtccttttgatcttggtgttgacggttttgagtcttttccattccgatttgacttttgtgcatttttggctggagtatctcatatagatttcttcactggcgctttttcttcagtttcctcatcatcaaaatcatcatcatcatcaaaatcatcatcttcatcagcagcaagttttacttttttctgtggaaccttgctaccacctccaggagcagatcgctttccagatatacttatgagtttcacatcctcctcctgttcgtcttctgactctgtatcttcctccccagctactaaatgctgtccactcacatgcactggccctgaaccacacttcaaccgtaagaccactgatggtgttatttcaaagccctcaagggaaaccatgggctgtacagacattttcaaagctgccagtgttactttaattggactgcctttgtaactcattgcctctgcttcaacaatgtgcaatttatcctttgccccagcccctaaactgaccgttcttaaagataactgttgctcaatttcattattatccaccttaaagtgatcatctttgtcggcctttagttcacaaccaaaaagatagttttggggcctcagaggactcatgtccatcatcgtccatcaggtggcaggacgcacttaggtgggagagaaggcagatgatgataaaggaccactgctcaagagaacagctgtgcaggacagaatcacaccagggagattacctttatcttagaaaacctgaacatcttgtgtactttgacacttctctacatttcacctaacctttaacatcaacacatttattcagaaaacttttacttttggagctgctctgtgtcaggctctatgctaggtgctcaggatattgaaattgatacaatcctaacctattcacatataatccaaggtttgctgaaattgatggacatttaaacaattgaaacatttaagtggtataattagcaaatggacatttaagccataaaaatagcatctaatagatataatagaggtcggtacaccattgatgagtcagagcagaggcaacccaaagagtaactagccagaagaattgggaaagcttcatagagagagcgatatgaaaataagggagagaattgtaaatccatgaaaatgagaaaaagttgaaaagtgatggtgtcagaaaaacttgtggtatgataatgacaagatgagaggaactcttggtaagcgtgttggatgcatggaaagaaatggcacaaaataatgctgaggacattttttattttattgttggttttgttttggttaatttcattttttaaatctagtatgctagtgttcattgtccaaactgtgaatcataaactcagtttgtggatcaacaccggcctttgatttttagtgaaacaaaatagaaaatatcagcattcatcacaaatagatgtttcacagattttttgttttaattgcgactgtgtgtgtgtgggtgtgtgtgtgtgtgtgtgtgtgtgtgtgtgtatgtgagagagagagagagagagagagagagagatggcttggatgtttatcacctccgaatcttatattgaaatgtgatttccaatgttggaggcagggcctggtaggtgtgattggatcatgtgggtggatccttcatgaatgatccctttggtgacaagttagttcatgctatatgtggttgtttaaaagagtatgagacctcaacccccacctgtttcctgctctcccctttgccttccaccatggttggttgtaaacttcctgaggctctcaccagaagtagatgccagtgacatgcttcctgtacagcctgcagaaccgtaagtcaaaagaaaaccccttttctttttaaagcacccagtttcaggtatttctttatagcaatgcaagaagggactaacacagttgtatgtgtatgtgtgtgttgggtgatttctggttgagtgtcacaaggttgtaatatggtgagtgtaaggaagtataagttttagaaaattaagaagccagttcagaaaactaatacttttggaaaatagtacaaaatcaactttacaagaatatacacagaaagatgtaatacaagatttatttcattgcagtaatttataaagttggtttagtgccttgcttttgcatgctgttttaaaaattaccaagaatatgacttcatgtgattttgaaatactcccagcaagataggtagaaaaggtattcttataactcttagacaaaaatttcggaaagtttaaacgctttatcccaaatcataaagctaataaatgaagaatctgggattcaaacaccatattttttttactgttcatcagctagaagttagaaatgttaagccaaaaacattaagtcactgctctgcctaataaatcttgaggaaactaataaaaagaataataccactgactacaggacaaggtcttcctaagagaccttaaatatattaagtgatgaagatgaaacttcttttattcataaaaatgttatttagttatgagtagagctctaattaaacttattttatattgtcatcagtaaagttgagacataacatatttattaatataattataatttgacccatagtgtattaaaagaaggatgttaaaaggagttgttattagagatgatgttagggttgttgatgataataacagtagtcataacataacaaagcacttcataatttaagaagtgccttcaattacattgttactctcatggtaatctctgtttgatatatagatttggcggattctatatcactctaagacataggttactgaggtgacggaggaatttagcaagcggctgtcaaatggaggacatgagcattggattgtgtatggcaagggctgatggtctctaagaaagcctcttggtttccacagggcagaagccctttgaagatcatagccaaggatttagtaattgcctccctttcagaataccctcaagagaaaagcccaccataagacatggttccctacaggcaaaactgcttttccttaaaatttactgttccctgaatatcagccttctttggctcattcaacatagttttcttaagtttcaggacagtgctgcagaccaaaagtttcaacattgaggaaaacaatactacttgtgcagtgaccctacctcagtcagggaggcagatgcctgcctttatgtgagggaataaggaatcaatcatatttccagcactcaagaaagccagtctagtgcagggagagatagatacataaacctcaaagttatgatatagcataatagttttaaatttccataataactgtattttaaaagttttatagaaacagaagagatgacctcagtctggaaaagccagcttggagaatggcaaccaatattaagtggcaaaagctttgggatcccaggcctccagatggagggtgatagcatgggccagacaggtaggttaggaaaactttgcaaaggacattacacggtacacagacaagtctgtgttttagcctataaaccacagttgcagaatgtgtttgagcaaaggcttttggggatgagatttgcacttttcaagatttaagtttgtttaggatacttacggtttgctgtatacttcctgggtttttacattataattacggtttgaactttaaaggaaaactgcagtttagcatacttgaaagagtgcaacttcaagtcatgattggagacagatatttaacagattttgtgatcctgtgatgcttattttcttctcagacataccacatgacaatcatttttaaacagtttatttctactttagcatccatctgaaggtgttgtgtatgttttctgcttgaaaataaagcagtgggctgggtgcggtggctcacgcctgtaatcccagcactttgggaggccgaggcaggcagatcactaggtcaagaaatcgagaccatcctggccaacatggcgaaaccccatctctactaaaaatatgaaaattagccaggcgtggtggtgcatgcctagagtcccagctacttgggaggctgaggcaggagtatcgcttgaacccgagaggcggaggtcgcagtgagccaagatcgtgccactgcactccagcctggcgacagagtgagactctgtctcaaaagaaataaaaaagaaaataaagcagtgaatgcgattaagatggatttattatgatcataaagtactcaggagtcttattttaaaagacagcattactgtaattaaaaatatagggaagaaactaatgctgttttgcgtatcattctcagctctctcaaaatcagatattaagctcttgctgccaaaggagactatactgcacggtgctcacctgcataaactttgagagggttgaattgtgccaagcaattctctcaatacataaattaaccaaatatttgttgacctactgtgtgacaagtattattccaggaaataagagatccagcaatgaaacaagtatggcttcttatagagttcccaaaaaggaaataaaaggatatacgtatagtgatatccctgaattaaatttctcttttgaaaataaaaattctatcataagctgtaactgccaacacttcaatactcattcagcagttttcagggatttgtaccttttgacttatgagaatttggaagtctaattgtatcattgcactggagtcttaaagaaacagataagcgaatgactttgcctgtatcattgttgactgtacttacaatcagaaaggggcacaggacagatgccagggagtaagtggacagcccataaatggaatggtaagaaagaagaactatagtggatttggaaagttcccttcagcattttccctagacaatctttggctgtgtttgcatgatcagtatttcattcacaggatattgagctcttgatatagttctcaaaacccaaaatgaaataagaagtctactctttatttaaattcaaattccagagagttaagtaactttccaggaggtaatctaaatatggcctccttgttggggggggggggggtgtttgaatttgcatataaatagtctcacccttaaaggaaaaccacagatggtggtaatgatgtagtcataatgtacatctccacagtggtggaacaaaatatccacagttttgctttccccagtttcagtgacccatggtcaactgctgtctgaaaataggtgactacaatacaataagatattttaagagagagaaagaaagatcacattcacatgattttcattacaatgtattgttataattgttctatttttattcatgatttttaatctcttaactgcgccaaatttataaattaaaatttatcacaagtacatatagtttatatagggctcagtactatctgcagtttcagacatccactgggagtcttggaatgtatcccctacagataaggggtaaaccactgtatcctatttgtgtgaatgctacaggtgttgtgagctcataacaatatgacatcaacactgaactaatccaggatttggtagtgagagtgatgtatttgcaaggagtgagacgtggtgcctcatccaagcagagaaataattttgaaatttgcctgacaataaaaatcacaatgtgaggtctctctttagagctgcaaagtccaattcagtgccccctagccacataagatactgagctcttaaaatgcggctagtactaattgagatgggcactgagtataacacacatgccagggtttgaatacttagaaccaaaaaggaagtaaatgctcatttattgcatgttaaaattatggttttattatagttgattaaataaaatatataattaaattgacttcattttgcttttaaaaatgtggctatgaaaaatttcaaattatatatgtgtgtgattacatatgtgtgttttcacatatgtaactgatgttacatgtgaaattgattgttacatgtgacatgtaaaacacgttacctaacacgtgcatatgtatgcaacacatatgtaacgtgttacatatataacacgttacatatgtattgttacatgtgtgcttgcattacacacatgcataatatgaaattacatgtaatttcaaattacatgtgtatattttgaaaattacaaattacgtattttgttatttttgctttacaaagtcaaatttaccctatttaataaagcatcatgagttttttataactagtaaactttgagacttttgtaggagaataaataatgcttattataaaaactgattggaaaagtgagctggagcagggagcggaggaaaaaggactagagatcacctttcttcccagctccgctcctctcccaaccttttttctttccattctctcatcccaattcaaaagtgcagagttcacagttggtgtgctgatttagaaaacagatatataaacagccttaaattttctccaggcttttacaatgaaaagaagttcaatatcaaaagtaacaatataatctgtggaaaggtatagggggctatgtttttgaggtagaaactataggtgctcctggccaagcatggtggttcaagcctgtaatcccagcactttgggaagctggggcgagagtattgcttgagcccagaagtttgagtctagcctggcctacagggtgaaactccacctctactaaaaatacacacacacacacacacacacacacacacacacacacacacacacaaaagccttgcgtggtggcgcttgctgatagtcccagctactcaggaggctgaggcgggaagattgcttgaacctgggagacagaggttgcagtgagctgagatagcaccactgcactccgacctgggtgacagagtaagactgtctcaaaaaaaaaagaaaagaaagaaagtataggcactccttatatgcagctgctcacacccctcctccttcacacccctcccccttcacacccctcccccttccccaaaatttgcaaggggaaaaatgtgtgtaattggcagtatttagtggcgtgcaaccgtgagtcatcagactgcacatcctcacttctgctagtggctcagtacccaacagcactcagtgaaaactaactcatttcaaaggtgaaaacaagtgagtttggccaccagggagtgttcaaaactgtcagtgctgaagcaaatgtggagggtgttctgtagtttgttcaggttgatatttgtggtccaacccctagctgaactactaattattaatatctgtcttgatggtgcctcaggagaaagcttctcaaagggaatcaatgttcaaattatagtaggtatcttggccatggaagttattgaattttagccaatacttgctactctttcatttatagtgtgagaatgcagtgtaatgaacctgactctcactgtcctgacttgcctttctcatcgcattcacaataagcacgtcaatacgtatacacatttcatatttctaaagtttactttatttccttattgtacatcgctgtgctgctgatggaagagaaaaggaaaaacactattgattgcaaaactgttttatctttggtggcttagattttttttgtatgatatgtaacgtcttgcatacctaaggcaacacgaagctaaatagatttgcatatagcatgtattttttccaattaaatgtttaattttgttcagagtatactggggacattttgaataatggagaaaagtacaaagaaaattcataattctaccacctatcagcacagtgaaattttatgaagaaacataattttcatgtaaatcatagtgaactcacggtaggttttatttaatacagtaattggagagctggtaggaagacaaaactggttcaaaagagaatacaagaaacaaatgcttctataatgagtgaatttttaaaaaagtattctggaataagattagtgaataagatactaaactcgttgataccctacagcctttggggttatatcctctactgggtaaaaagtcatttacatcatatcagttttctaaaatttgcattgaacttcatagcgttgtaacatgtgtgggcccaaattaatagtaaacagtaagagttgctttactctgaaaatattgaagctcttgtgagggtgtgaggagtttgttagaaaacaacgctaccattattttgaaacacacacgatcatcttttgttttacttctaagttttggataatttttcttaaattatcttattatcttatccattttcttaatttccttaaccttttaaatgtttctcctaggcacttttattgatttttggaatatagttgatatgtgctgaatttttatcatccagttttaattctactgaaaaatctaaaagatgttcatcaactactatatttcaaatgcatacatcccctttcatgctaaagaaactgtatgggaaacacagtctgacattttcaggacctggtatcattaaaagtcttgacactgttaaaattaaacaacgccttttttaaaatcaaaggatacaaaagggctgtgttggtcagaggatacaaaatttcagttagataggagacataagttcatgagatcttttgtacgacatagtgactataattaataataatatgttttcgaaaattactaagagagtcgattttaagtgttctcaccgcaaaaaaatagtatgtgaggtaatgcatatgttaattagctcattttagctagtccacatttttcaatacaatgtgttgtataatacgtgatatatacaacttatattttccaattccaataagtaaaaataaatgtaaattatttgaaataaataaaatgtgaagaacatccacttttcatatgaaaccatgagatattttctgttaaaagattaaatgtccaataaatttttgatgttaacagaaacaaaaatgtttaatatttaaatacatatttgcatgctattgaccccctgaagttcactgctgggctaagtgaaccaactatatcttaagtcaaaaatgctgaaattcttccccaaatcccaaagctcatgaaaacataaacagaaaatttccaaataattctacagggaaaataagacacactatttgatctgatcaaacaacgggatgattatggttaataatgagttacttgtacatttaaaaataactaaaggagtgtgattggattgtttgtaacacaaaggagaaatgcttgaagggatggataccccgttctccatgatgtgattattacccattgcctgcctgtgtcaaaacatctcatgtaccctacaaatatatactcctacgatgtacccacaaaaattaaaataaaaaagagagggacccgaagataagctaatatttaagctcatcatacttattaagataagcaatacataccgaaagtaatagcatttaaaaccagatgttgggggagggttctaacttgttcattaaaattcaaagtcacctgtcttgttttttcttttgtttttgtttttttttttttttttttgagatggagtctcgctctgtcaccccaggctggagtacagtggcgcgatcttggctcactgcaagctctgcctcccgggtttacgccattctcctgcctcagcctcccgagtagctggtactacaggcgctggctaccacgccccgctaatttttttgtatttttagtagagacggggtttcaccgtgttagccaggatggtctcgatctcctgacctcgtgatctgcccaccttggcctcccaaagtgctgggattacaggcgtgagccaccgtgccaggccacctgtcttgttttatcatgatcccgagagtatatatgtatgtgtacagctcatctaaaccctttttctttcaacatgatcaatagattgaacattggagatattttataagaaataatgaagacaactcaatcagcacatatatatattaaatgtggaatctataatgattgcgaagcctgaagcaaactaaatattcagtaataggttctttttttccatggtatatccatttgaatatataacataaatgccttacatttgttttaactatttaaggtttatgttgttagtgtgatgaaatggctggcaaaagtcagaaactcaggaaagtttcaggcttatatctggagcctggttttctttcttcaaggtagaacctctgtgaagtgaaaaattttttttatatctggagcaataatgtagaagcttaaatgtattatccaagttgtcataagcctattatttctttacattactgaagtgaaagacagcattaatggctaaatgccatacttggctataatttatattgtttaggactggaaatgagcctgaaatgtacatttttttccaaaatagttcatgtaatatttgaaacctgacaagtaacctgatgatttcatggaataccatcaaatataaatgtgaagttttaaagacacagggaaatactcagaataaaccccctaaccacaggccagcagaagaactagacttgagaaaatgaatgggaagatagatagtaacaaatgacttctttggcagccttatatatgcttagtcttatagactgttttatggatgctctgcactctatttccagcaagtatggcatttggaacaggaccacacgagacaaactatgagttcacatttcccacaactgcacagatagaaagagggaacaacagaatactccctttcttcttgaaacaataacttctgttgaagctcactggcttcttttcagctgtttctgctagctcctcctccgcctcttgacctctaaggcaatgctcttcaaaatttcaagactgctttctaattgaaacaaaacttataagcacatttcttcccacaaaatgtacatttatttgtaaatcatatatgaatatgactaagcatgtaaacgtatgtgaaaatagaaatcaataaatataaatgcaaacacaaatagaagcattcacagttttcttttgtgtcccagtgagttgttccaaattcctcggaggtaggtatgtcacagtttgagactataccttcaatcctagggtttctggtttcgctctcctcctaggtgatagcatccatttctacggacttaactgccatctttagttgaataactcctctatctttccatcccatatttctcttgattccaaacctgcttgttcacctgagcatatgacacaattcattggctgccgcacatgcagctttgacattttatttaaaatctttccccttccccagccctcatctatttcacagtagtatcttcttcttatctacttgattggtaagcagagtccacatgattccatcatttatctcccattttatatctaatctataagcaagtaatgcaatgcaacttctgtctccaaaaatttattttgaatttgccttctcttcctctgcatctcccccatcttaggccaggtcacctctgccctcttgccagattaggtcacattctcttactactgttgttattctcttcctattcaatcctacaccgcagcaaaatggatcttctcaaaatgtcagctagataaaggcatttctgtgcttaaggccctcatggatttatcttattaggatgaacacccaactctttattatggcttagaatacaatgaattacaacacataatgaatatattatatttctatctttaccattttcttcttaagtcaacctttctcaatccatataggataatcatattagtgcttcctcactttctaaaacatctcagggcctttgcacgtgtttctctgttcttagacccagaatgctcttccttttctctttgtgtagctaggtgcttctttccatttacgtatcacatgaaatgcagtcattccctcctccttccctcactacctcacaaaaagttgatgcctctgttaaaccatgaatggaattttactcggcagtgaatagaggaaaaaccaatggtaaaagcaaccatatgaatgaatgaatgtcaaaaatattatgctgagccaaaagtcatagacacaaatatgggtatttacatgaagttaaagcacagcaaaactcaattacggtaatagaattaagaaagtggttacctctgggtgagggttggaattgagtggacagaggcattagtgactttttcggggtaatggaaatgttgtctattttgttcaggtggtgaatacatagatacattcaattgtcaaaacacatccatccaaacacttagacttttgcactttattatatgcaaattatgcctcaactgaaaaaagtttgttttcaaaattatatcaacagttgaaattcttttaaagatttgattcaaatgagattaattctgtatccatcattgatgtatgatagttttgtatgtagttaaggttattggagataattgaaagttatactcacaagaaggctgcataatatgaagtttatctgccttgatctttaatagctttcgcgatttcaacttcttcacagctctgtaagaaggcagtgtggcatgttgaagcaagcatgtgttttagagtaacacagagctggtatacaaccccatgtctaccaattatcaatgatgtgggtatgttgctggatctcaataatcttccactgtgaaatggaatgtaacacctgactcacaacgcaaaggtatttaccttatgtaatataattcctgcgatcctgggacctcccttaatcccatccacagatgccaggttaaagaccccatcacagactagaacaagttgggatgtcaaaatgaataaatattaatcgaagggcctattgtgattgaacaccacgcagtaggcactctctaatacctaccgtctccctcctttttgggggaaacattctaaatgtgcaaaaaataaagggttatttgctttctggcacttgggatcgatttattgaggatatgttagcagaacagcaaaggtgaaacactaaaagcaccatcaatacacaggcagaggtgaagccataaagcctttattttttaaattaatgcacaatatataagaggtatgttagaatgaacgtccaatccctgaaaggatatacgaaagacattcataaaattacatgggcatgttttcttaatgttcaaaatattgttttaattagtgtattatgagtttattcatgtgtctgtgtgttgtgttatattaatcttttcttgcattgctataaagaaatacctgagactgggtaatggatgagaaaagacacttacttggctcacagttctgcaggctgtaccggaagcatagcagcatctccttctgtggaggcttcgggaagcttccagtcgtggcagaaggcagaacgggagcaggcacttcacctggctagagcaggagcaagagagacagaatgaagtaccacacacgtgtaaacagccagatctcagagaactcactcatcatcatgaggatggcaccaagaggatggtgttaaaccattcatgagaaatccacacacatgatccagtcacctcccaccaggccccacctccaacactgggaattacatttcaagatgagatttgggcggggacacatatccaaatgatatccatgtttaatcagaaaaataaaagttaacagtaacagtgattttactttgtagacctttgctaatggctgaaatctagctccattccgagaacagcctgcggtacacattttgaaagatagttgattaatatgaaagaagccttatctgtagtccttaaggccattatggtttacatatatgagtaaatattccaaagtagccatgccagttaacatatatccagagtctaaaggccactgggcgacaaaagtaaaagatacatagcaattgttactttatatcacagtaattcttgtatattttaaatggatatttgcatttgaggatatccacttaagagttaggtacatggctcttacatttaagtaacatttacttaaatttctggctgcagcaattccacataggtagaaatgaagtctgaattgagttgggggtctttgcagtgctctctctgttcattggctattttgacaatgctgagagatgtggttagccattctttttcatttcatattggcaacctagagagcaattaagccttctccccttaactagatgtatgttttactcatttctggatctttatggctgactttgaatcctagcctgtggtagaaagcatggtgtcagaaggaactatgagttaagactatgcatacttggctttgagtcttgggtatcatacctccctcatagagtgaaggaaccagggattcttcttgaggcccagacccggcatccatgttaagaatacctgtgcaattttgcttcctgatatttaaggtgaaaatgcatgtttgggtcattgtgaggattatgtgagatgttacttttaaatataggcccccttattatatgctctcatagtttcaggcaacacttgtcgtatttgtaacctcagttttaactgtaatgtttccatcaatgtccctcttacctggtacaggggctcttcatattcttggattacaaatctgtgaatgcaaccatgcatcaaaaatattcagaaaaacaatgaatgcctacctctgtactgatgatttataggtgtttttcttgtcattattccctaaacagtacaatgtaataagtatttatatagcatttacattgtattaagtattataagtaatctagagatgttttaaagtatataggaggatgtgtgtaggttgtatggaaatagtatgtcattttatatgtcacttgaacatttgtggatttgctatccgtggggatcctggaaccaatcccccatggatactgagggacaattgtattataagcagcaagagggaaaggaatctgtctattttgcccaaaatcgtgttcccgggacctagcatagctcctggcaaagagtatacaacaaatatgcattgaggagagaacagagggaaccattatccccttattctcgctgttccttcatgtaatgaataaacagtcaaatcttacaagagattttaaaccagtcagagaaaagttggaagttagttagttgttcatacattgagaagcctcgacgctgtgtcatctaggtaatgaaagatctagggaagtttagcagggagaagaagagagatgatagttgtcttcaaatgtttgaaggactgttacggacacaaaaatttaaacttgtgctgaataattccaagaggtacacagtctctcgatagaagctaaagtggggggtgacatttgactcaacaaaaagccatctaaatatcagaactttcaaaagcaggaactggtgcctcaattaatagtgtgttttctagcacttatgatacctgatcataggcaagataatgaaaaattgggacctgggagttatacatgggaatttgtttatcagttgggtgattaggagaggtggccttaaagtcctgttgtgttctaagagtctgtgattctgagtcttatttcccaacaagagaggtacagagcagaagatgggattgggagaaataggataaagataccaggaaatcctaaaggtaagaaaaggaaggcagacctgaagctaactctatacttcaggtgcttgcctagagccagccctacctacttagagaatgttgaagagccagttaaaacatctttaacacggatgtaaaacaaaactatcaaaacctgaagatttcgaatgttctaacctactcgtcagttgggcttttttcacaaatacttcagtaaataggcataaatttattttttaatgatagaaaatatctcttaaagaacttataactgtggataaaagcaccaccataaaaatcttgtggtgaaatatatatatatatatatatatatatatatatataaaattttaaatatggttagctagaatatgacgacaatgtttatgaaacacagagactcttgacaagtcccatgtatacactataaaactttaagttatccactattcactcactaagcttatacttaatgagtgtctgctgtgtcacttattgcggaaggcacaggcggtatagcattgcacaaaacatatgtggtctctgatggagtttttcagtctagtggtgaaagcagtgaatgggtgtacagatgttaaataattgtacaattagttgcatgtgtaaacgtcaaagttcagaagatgacaattgatctacggcaatgtttctcaatctctgacgttttgagccaaatacatctttgttgtggtggactgccctgtccactataggatgtttggcatcacaactgacctctgcccattagatgccaatagtactctcttctttaatcacaaatttgtcccagacatttccaaatgtcccttggggagcaaaatcatccctagttgaaaatcactggtctagggggaggtctttatgaggaagtaacatctaagaaagctggtatgtttacatatagctacagtctattacacatgtatacatatgtaacaagcctgcatgttgtgcacatgtaccctagaacttaaagtataataaaaaaaatgtaacaaaacaatacagtatgataagtgctatgggaccaaagatgaaagggttctactgcacagttatgaactcatagttaggcttttggggtcaaaattttgctgaagatatttgccacccacgtgacctttggcaggtgacttagcttattcatgcctcagttttatccaatgtgaaatggggctggaaagtcccatgtacttcctaataactttgcggaaataatatgtggttatataggaaaaaaaaaaaaatcctagaagtatgcctgctgcgtagtaaaaggaaggagaaggataaagagaaatctgcattttttcttctgtaatggggcagatagtaaatattttaagttttgtggcccaaatagtctctgtcacatttacttgattctgcagttgtggcattggaagcagctatggacaatacttaaattagtaggtgtgcctgtgctttcaataaaattttataaatacaaagtttgcaaaacaaagttgttttttttttttttgtagtttgctgacaccctagtaaagaagcaccattgtcaacgttaaaaattatcaaatttttatttttcaaagttttcaaatttgctttgcttggtctagctcatgaaataagtcaaaagtagcaagacctccacctctaaaataataatagtaatgataacctcaaaaggaaagaagaaatatttttaaagaagaaaaattattgttaaataggattattgtgcagagaaaacctaggagactcaattttaaaatctgtgaaataattttaaaaatactttatgaatagatacataatagcttttattcatattaatgactataaatgcaaatggaaatatttcattcacactgatgacaatgtataaattaaggaggaataaaaattgtagaccctataggtgaaaagcataaaaatatacataagaaaaagcaaaaattgactacgtaggattgttttaggatttaagatttattgtcattaaacttgcaataccagccaagttaacatttgaatttaatacagttataatcagaatgcttttgatgtgtttgggggcaatataatttcaaaggaaataggcaatgatgtaatttaaagtttatatagaaggaaattgtgtgcgtgtatgtgtgtgtataaattggaaacaattttattaataagcatattatggcagcaacatacacttccagatttctactatactttgaagtaattgtgatcaaaaccacagtgtgctggcataaggctagagaaatgggttagtggtttacaagtgagagtccaggaaaacatccaaataagattggatattttagttctgtgtggatagcctatttcacttaataaatagtgtctcgtaattgactattcatgtacctataagtttaactatagaccaaaaaaacgccctactagattaaggagctaactagaaatataaattcatataaacaataaaggaaagtgtaggactttataagcttcatgggagacagatttttggtaagtcaggaagcctggaagacttaaaacataaaattggcagactgaattaactgatagtttaaagcttccatagagcaaaataaatcataaaccaagttttaaaatatataatggatttagagaaggtatttacaaaaatatatgactaatggaggttaataataacaatatgtaagaaggatatgaaatggcattttactataaaggtcaaacaaatgacctataagcataataaatcatattaatctccactagtaataactacacacatctacataatatagatgttacgcctgcatttgatttactttatctgtcttttggcagaactatttgtcaccagataaaaaattctatatcattaccagaaaggtatattattataatgtttattatgttgcagttgtaaaagaaataacagcttttcaattgtttacaaatcctatagaacatttactgaaatacatttacattttgtggcaaacttggatttaaataccgtgttcgtgctttgttttatgccgttttcccatcttttctccaggaatttgattgtgcttcattgaaagctaaaaagaaaaaaaaaataattctggttttggtttaaaaaattaggttaggggttaaaaagttgtacgttgtcttctgtaaaaataaaaaacaagttttctttgtttcttggaggctttatattaaatggatttttaattcatagacagcatattgtgatgaaatttccccatgagcttcacattttgtttcaatagcagaaactaacttggttgcagttactgcccttctgagaacagtgttctggaataattttgacatacatatgtatctctttttaaaacatgtgttaatcttttcataaagaaagttttcccagctgtgtcacctgtgactccaactttctggggggacagggatatgagatgttggaagggaatggcttgaagaaataaagtgcaaaagacgtaatgctttcctgtggtagaaatgtattcagtgaccctgaatgaccttcctactcttgtcccttcatttttcccacaagtatggtctgggcaattataaaaattgacatttgcagtgggctcttctgtaaaagatgctcaatcagaaatgatttattttagaaaaagagatgatataaacatatatatcccctgtctcggaagtgtgaaggttgaaaagcaaggagatgatcttcaaagtgtctaaaatattgatttgtaacatcgttttatgaaagtgcttcagattattttttttcttggatggccccttatgctttggtcagttgatgctaaaatctgaacttctttattttaaaaaaaacttttaattttgaaaaaggaagttcacggtgctgtctaattctttttagatagtcattaatgtaaatgtaagagtcattctgagaaccacatctgctgatatgttccgttaaattacaagttctatgtgtatttgctttgctttcatacaatgaatcttctttactctcttccccacctgccagaaattgccccactcaacgttcataaaaggtccattttcaatcgctatatttatttcagaagcagagatatcatatattcaaattttagttactttccaatatcaagctaataactcacacaaataaatcaaactacagcaaaacagcaatctagcattcaacaaaacctccccaatgcacatatttcaagctgtagatatgtatcatccaccatgctgaaataatgtacatgttcaaatcaaatggaaaactagaatcaaaattgttgattacttcttatcagggcattttattatatttaagaaaaatacaaattaaatcattttcaggaagcaatccttctggctaagatttttttagcataatgcttaaagttaattgttgatctttatctataaattcaaaggtggactaaaaatgcagaatcaatcaggtagtccattttgcatcaggtgaaatatataaagcataaaacagcgagttacatttcctaacaaaattgaattacagtgagtaaaagtgacaggacaaatgcattaagaaaagatggactgaaatggatagagtagaatatatgcatctataaaacacagtcatatataatacactcattttttttcttacgagtgtgagattaatggaagaaaacaacaataataacaaaaccagtgtgatgtgtcagatttcaccttttaattaaaaaattattcacttcagaggggaattttctttcttgggttagctcaatcatgtcagatcttgttcatttaaaaggtcagtttacttgccttctgaggtttttgtttgggaaaaagaaaagaaaatagattttcattggtatcctgggtagaattaattgtttatcattcatttttaagatctccgagaggcagaaaaaggggaactgtgcaacccttttgtccttctggatctcaaaatgaagggatacattctgctacatgaaatgtggaattaagaccatgatgcaacatgataaacaacacaaatttgggggtgtctctgtgctatacattattgaatttttccatgctatacactttttggatgtgtctgtgctatttattcagtttttttaaataaaagtttttgtagactaaattgccctctctactttgcatcgtttttgaacaaaggattttcaagactgataagctcaaatgtatcatttattgtattcaagtagcattcaatttttctttagaagtataatttgtagatattttaacacagaaaacttgcaacactgctcatgataggcacttattatatattttttgaaagactatatggataatgattctaactttgacttttcctgttttgccttcactttagaattaagcagagaatcaaatccatattcctgggggcgatgcttggacaacagtatctctttaaagatctttgtgtgagtcgaaggtgcagccagactgggagttattgtgaagaaacagattcaggaaggttgagaaacttgcctaaggctaatcagatagttactggcaatgttgtttctaaatcactgtttggctccctcattcaatgaatctacactatgtgggactgcctcttgctcctgacatcttttgctgctgaaataaatgaactcaaagcctagaaggtagaaaagagggagttcagaattatattcaggcacaaataccaataaggctattgcccccagaactgcaacttctcttggtttaacagataactatttagctgtgaggtacaactgaggaagtggacacacaagttatcaggagattctgatgtgccagtttatatttcttgtcacaggtaatgattcgaaatttcttaaaacagctgtcctcacagtggagtaacctgggagtacatgaaggcattccaaggagtaggcacagatagttttaagggaatttatttctagatcttctactttattttgtactcttcctgaaaactgaattgcctgaaaaaaaaaaaaaaaaaaaaaagacatctgtagtcaagacctcaggctgtttctcctttctaaccacttgccttttctaaccacttctcccaatttaagaaaaaaagccttatatttcatccaactctgatcttactaaggcttcaaacaaaagaagcatgaatgactttcatgacagggcaacatagctttttgcaagaagagtggttgctaactctttgctttcaactgaacccgaagagaagacctgataagttgtcagccgatagatcattaaaaatacgttttggtaagcaatcatcatgtacttttagcatatgccatagcaggagcacaaatgattaagcaatgctactataatacaattccttccgtttctttctactcacctatttgaataagatttttcatcatttacatctatacagacaaaaattagggatagaattgatgctgaagcctttccaattgtagaattaatttatattcttctgaaggtgtataaattgttaaatacccatccatcttattaagagatgtattttcaataaaattttatttttatgtttatcaaattttataatatacatatattgttttggtcaattgcacgttaataattgtaacaatacctcaattgaaaaggtttgttttttacatttaggacttacagtaacagaaaaaaaacactcattgtgtatacatactgtttaagaaaagtatactaggtgatcaataagattttttcaggcataaacatatatcttagttttaagatatcgatatttacaatgtccctcaaattatattattttcagtcatttaagaatgaaaagtacatttcgaatgcggattttaaatctgcaagggttgactcatttttcaagagtctttttaggggatacagaagcaagaatgtttggagttccctgatcagtatctttaagagaaggtatttgttggtagttcctagcaaattccaacagcctgatgctacttaaaagataatagtaattattttaaataatgcttctgataaaaaacattcatgcacactcagtttaaaaagatatttaaacatttgtagttgtagtttgggaactcatgatacaagtacagtctgtaaatgaagctcttagtttgcaaatatcagagataagctattaaaatgcagaaattgaaattgccctgatatatgcataaattagtgtcatctccatcttgtcagttagagtattttttagattctctctatgtatacatacatatatatatatatatatttatatatatatatatatttgtgtagctgtgcatgtgtgtatttggactaatgggtcaaaggacagtactaacccaattcaataattaaagaaaacataattttgagaattagctttatggtaattgtttgacttaaatgagtagatcagagaagaataagggctttcccttatttaaacaagcttcatttttttatccaaacatttacttagctgattaagcttcacttgtttattttcttcaaagcattcattcaggtgggtactgagtaaactgaaatatcacaccagggaacttcaacaccatccaagtcttaaaggcttcacttgttcacagttggcatttagtgaatgtctaggctactgataatattgtgagtaagttggcagggatcataagaaatgataaaatacagttcttgaaaatgttatggtttgaggaaaagatctatgtttggaattagactgacttggattcaaactctggctgtacctttgggacaaggtgttcagaaactctagcctatgttttttttctgcaaaatgatcctcttttccaggattcctgtagagattcaaagatatgtgaatgtttagaaaaagaatagacttttgatcattgttaattcccttactttccccaattagacttgtaagactgggaagaaagctacacaaaagattgaacaaattatagctgacagaccatagcaaaagatacagggcaaaacttaaaggggaaaactacacattaaattattttaaaccattaaatagcactaacttttgtcagatattacaaccaaacaccactcaaattaaagtaaactgaataaaatgcctgtttttttctgtttactgatgttttcatttgcttcattcatttattggaagatataaaatgtgttagacactgttaggtgctgagtgtataaaaaaatcttattaatacaatttaaacacgcacacacatatatatggttataacaattgatgccatgtatgtactgtttatatgcctatacattattccacagacctggggggagggggatgtagagtcttaccagaaccataggaatcttctcacatcaacatttccttttgaagtttgttcatgaggcaccatccagataatactaccatctgcaatgtggcttgagaagatgttagatttttttattacacataataaggctgtaaagtatttctgtatttaggtagaggtatgtaatacaatatgtatataaaattacatatccaataaaatctggtgttaaataaggactagcttctatgataatatagtctaaaggcttttcatttggtgttatagaaattatgtgaaatatgtttcctggagtagaattattcgcatttcagctctctgacagtggaagaaaagctagagggagaggtgaacaagagagggagcataatggacaaagctttgctggaagccaaaccaccacttcatatgtcaaatctgacaggcctcccattttaggtgtgctgtcattgaagctttcagctgcaccttgcctgtggctaggctattttcaaagattaaaatgcgaaactggaaattaaatgcaacttaattcccaatttaaatttccattatttttgaaaagtaaaagattaaaagaaatgtataattgcaattctggtggaagaggtaattataggaaaggtgggatgtatttcaagtgggggatatagcttactgcagcagagaggaatctaagctatcattcttttgaaattggtctggaaatatgttttcacatggaaaatatactatatttttaggaatttccttgtcatattactgtatccttttctgttagaatataaattctgaattccctattccactgtagatctgcctccgattatattagctcttctgaagttatcaaaaaataatgagatatacaatattccatatatgtcaaagcaattatttttaggttaagtaataaaccaatgacctttaacccggtaatattctgggttgttcataaaaaaactatattcaggtaataatgtctttccacttaagcaactgaaaaaatacacaatacttaacatttggttaattaaatacctactccagacaaaaggattttctgttttcaagttatcttagcaagctgagcaggaagcaatgatatatccaatcagaatatccatggaagctctgctacagtttcaaaaagttctcatcaggcagcttttaaaatgcctactctgaaaatggtccaggttaaagaacaacagcttcctcgtcagatagcagtattgcttggccatgtttcttcctagcacaaaaaagtacctgctcttctctgagtacctacattctaaggactatggcttacataaaacagcatgggttggggcaatttccagcacactgctcactctcgaaaacgtatgatgcaggtgagagtaatgtttttgtttgaatctgctttcactcgtggaagatgaaactacttgcaaagatctgtactttagctattatgagtaacaaaagactcctaaaatattgcacacattgtggggatggagaaccatcatcctgggatttgatggatcctatggtttggctttgtgtccccacccaaatctcattttgaattgtaatccccacaatccccacatgtcaagggagagagaccaggtggaggtaactgaatcatgggagcaatttctcccatgctgttctcctgatagtgagtgagttctcacaagatctgattgttttataaggggctcttcctgcttcactgggcacttcttcctgccacctgtgaagaaggtggcttgctccttctcaccttatgccacgatggtaagtttcctgaggcctccccagccatgctgaactgtgtgtcaattaaacctctttcttttataaattacccagtctcaggcagttctttatagcagtatgaaaatggactaatagagacgtgtctctcagaagtcacagtgatgcttgaacggatccagagctccttcttcaggaaggtcccaactcattctgaagggtctctccaagcccacctctctctgtaaatgggaaaggttttactttgagcactaaaacctgccagaattctcaattttcctaacagtgtgttaataaacacctactcatttagtatccaaaccaggtctgtatttctcaattagagctcaccaggctttcatcataaagtagagcttcaaattgtctgcaatcccactcctatcaaaaacctagaaggaggtaatatttcagagtaatactataaccagatgaccacatctaagaaactgctgaccctacgatgtaaccttctgtccatttttccctttggaaagtctaggatcttttcttataccagcaagttacaagcctggactacactaacttgctttccgcagaagaaaacaccatgagttctgttttcatattaagcacttagtctccatcagacatcaatcgagaaaaaatcattaaaaatcacattttatatttgatgtatatttctcaataatcctatgtattagttcattttcctactgctatgaagaaatacccaagactgggtaatttataagtaaaaagaggcttaatggactcacagtctcacatgactagggaggcctcacaatcatggtggaaggtgaaggggtagcaaaggcatggcttacatggtggcaggcaagagcgtgtgcaggaaaattgccctttataaaaccatcagatctcctgagacttattcactgccataaggacagcacaagtatttagctccctcagcacagaaccatccccgtgattcaattacctcccaccaggtcactcccatgacacatggggattatgggagctacaattcaagatgagatttggatggggacacagccaaaccatatcatcctatttggatgatcaatattatcaaggtatgctcccctgagggggcgtcctttttaccatttaactccaggacaaaagtttatttctttgtaaggacagtgtttatttcttatggtcctattttctcctaagatccagacaccaaaatggccatctatcattgacttaactcctgaattttgcttagagtaacagatttagtgaatctaaatattttctggctgtggaatgttaatttatacatgttcaagttacctttgattcatgtgacagtttgtgccaaaacacactcattatcagaactcagatcattatgttggctcttgttttcgttactaaaggaagaaaaacagtttctcaaaaagaaaattctgatacctaggaagaccattatacctcactcttttctttatctcatcaccacatccaatattataaaagaacttacaaagtaaaaagaaaggtgttctgtagatgtagcgcctggcttgtatggtagcttaaatgaacacagctaaaaatattttatggctagtgtccaaaacagtctggcaccagacaaaataagaatatttaaaattatattttagagttactttaagaggaagggagagagagatgtaggcaggaggaggaggagcaggaggagagggagagagagagagagagagagagagagagagagagagagagaatctggggtttctatggaagggctaagaatatgtagaaaacagtttacaaagaaatatggtccaagaatcgtgtgtacacacacacacacacacacacacacacacacaccccctggaatatttttcagccttaaaaagaagaagatctgtcatttgtcccaacatggatggacctggaggaccttatgctaaatgaaataagccagaccaagaaagaaaaatattgtatgatctcacttatatatggaatctttttttaaaaaaggtcaaatatatacagatagtgaattaaacagtggttaccagggtcagggtagttgtgaggaaatggggcaatgtaggtcataggatacaaatgattaaaatatattaatatattaaaagatataatatacatcatgaggactacagttaataatagtgtgtattcaagatttttgataaatgaatagattatagctgttcttgccacagagtgaaaaatgggtaactgtgaaatgatagatatgataatgttctccacaatggtaactattttacactatatatataaatatctatgcatcttacaccattatgtggtatcccttaaatatatacaataaaatttattttacaaacacatattaggaatgcatattctgatttttaacaatagttaacctcattaatatatttcacactatcatttctagtgtacatgaaaagtagtttattgacattagttgtaaaaaaaaaaaaaatggtcttgagacttttgggtcagagaatgttctggccataaggtaggtttctgcttgcctactagatatcttaacttcgatttcctgaacatcccatcacttcagaatctctcaatcctttctaacatccgcaacattgtttttctttctgcatttcttatattgactgatggatttataattcactttctctgaaaaaccctgcagttatcatatatccctatccattctggctctttattgcccaaatctctaccaaaatcctgtcagcacagcctctgaaatatttctcaaagcatttataatctggctctcatcaacattttcaacactctgttttatcattccactattttacatcatttcattttcatttttaccacaatcactcatccaacaaataagtatttagctccctcagtaattagtattattattattaattataactagatgctgagcatacagaagtgaacatgacagacataatcccagcagggatgtcagactttatgcaagtaatcaaccatgatgaatctcatgagattctgagagagagagagagagattgagagagagagagaaaggggaaccactggtgtccgagttagaaatttgaattagtatctgggtcaccaaaagcttctgtgaagaagtgatatagacttggccacacaaaactaccgtgaaggtggtggaaatttttctatgcagagtaccacatttaaagagctaagcctgagagtgtcagagataaaggaacagaaagaatgtgacagcagattatgtttggaagaaagatgttcaagagaccaagctaaagaggagatggggctagaacctggagggtccttcgggtcctgttgggagttttttctctgcccagaagggctttgtcacgtggttgtcaggaaagagtcatgattagagctttgattcagagacttctttcgctgaagtgtggagaatggttcagagagaagcaaatctgaatggacaaaagaggttattattgtaatcttggcaagaagcgatggtggtcttgactaaaatagttctagtgagaatgtgacaacaaacctgagaaaaatacaggagacgtaattgacgggggttagtgttaagttgaacgattgcagagttgaatttgaggaaagtgtcatatatcattcccagtttctgatgtcatacacctctggagataacactgccatttcttttgaaatgggaaaataataagtgatcagtaagtacgtattggataaaataatgaatggttaaatgcataaggggagaggaaaagagttgcagagaaagagagtaaacgtattttggatgtgttaattttgagatacctttgaaaaatccaagtgaggggttgggtagtcagagaaatgaatgtggatgtcaggacgaaaggtgaccgtgatgaactgtatgtcttcctctaagcacgttatacagcttcatgtcacaagtgactcacttcatgtcacaagtgactcacaaggtcacttgtgacaagcatttgcctggtgcttcatccctaacctccctttctatactcagctaaaatgtcacctacaatacttcttccttgactccaccgtccccactttactgatatgaatacattttaataaaatgatataataatgcttagtttgtaaacctaatgttcctcaagtggtataattatctgatttgtatgtgatcatcaacccaaccatattaggagcaccttgaaggtagaagatttaggttcatgcttaacaccacatctggaccactgtggatttaactttctacaatgattgtattcattaatatattgggtgcccactatattccaagtaatatcctgcacactacgtacaaggaagcataggtcccgtgtgctcatgaaactgtaattttagtaagcagggataggatacaaactgagaaaggaaaacaatttagaaagtgggaaatattatgcacagaattaataaaaaagagaaaaatcttgaaaaagtcttcaatacctcacttggaaggtgattttgaagaagaactgatggacaaactagagtcagccatgtaatgatgtaggggcaaagcattccgggcacaagggacagcttatgcaaagaccttaaaaatgaactagctttgtatgttggagaaggataaagagaactaaggtatctataaggtaattaggaagaggatgagttatttagtcccttagtctttgaagcacattatctcatacttcaattgagtttattcttagtgtcattcttctggatgcaatatttgagataaatgtcttaatgaacgttcacctccctccgtagtaatgcctgagtgtcacaaaaactttttttgtttacatacgtagccatctaatggaaacataaaataggaatcaaaagttgagtttcatgtacaaaaggtaaggactgtacatgtggtcataacaacttcaaaagcacctgaaggtaacctttaaggaagatacaaaggctaggaaatatctaggatccatgaagacagacttacttaaggtcatagtgtgtccagagttggttcccgccggtgggttcgtggtctcgctgacttcaagaacgaagccacggacctctgcggtgactgttacagctcttaaaggtggcacgaacccaaacagcgagcagcagcaagatttattgtgaagagcaaaagaacaaagcttccacaacgtggaaggggacccaagcaggttgccgctgctggcttgggtggccagcttttattcccttactgtcccctcccatgttccatttctgtcctatcagagtgcccttttttcaatcctccccacgattggctacttttagaatcctactgattggtgcattttacagagcgctgattggtgcgttttacaatcctcttgtaagacgggaaggttcctgattggtgcgttttacaatcctcttgtaagacagaaaagttccccaagtccccactcgacccagaaagtccagctggcctcacctctcaatagcattaagaatatagtttcacgagcatatatgaatcaaaacttacatttgccaattttatttgcttgtttatgtgtttccaacatgtcttgtcttagggccaaatgtttccctagagaataactattccaactatcttagttgctgtatttttatgcaaccttcaactctccatactaaaatgtctccagaatagaaaataaatcttttcaaagtttcaaaagaggctctctatatattccccttaaaagtaccaggcagacatatttctaggtttctaacattgcgtgttgccaggaagtatatccaaaccatcacaagttattcatgtaaccaagcacacttattggagtgcttctgcttctgttcttgcttgaaattggaagctccttccaggaaaaaaaaaaaatatctatagaaggggaaaaaagtaattttactttgaaaataaaatatacgtgagcaatagttttattctgtttttaatttaccatagcttccaaagacaacattgttttatagtaggggttagcaagtgttttctgtaatgtaaacgtaaagggccagagagtaaatattttaggctttgttttctatactctgttgcaactattcaactctgctgttagaatgttgaagcagtcatagacaatagagaaatgaagatgtgtcattgtgatccaataaaactttatttacaaaaatggcaatgggctagttacggcttgagggctgcagtttgcagactctcacttcagagctaacagttgttgtcaggagtcacttgtttttggaaacctacaatgaggtactataacaccaaaaagagttatcccttcctttttctctctcactttttgaattatgagaagaattagaaatgtagttaatgataatgtccaaccagtgtaattatacttgttagaaacacagctggaagcctgttgtccagtcttatttctcctctgtgatcctcattttcagaggttgaagtcataagtttgccatgtctactttctgacaggggaattataataatgtggagtcaccttttgtttgtgactttgacaatgcttcattgacttactcaccaattttctaatttttatgaagactttttgccgaaatgtagactcagtcttctctcttgtctactctttctataacaattaacaatgaacttatttacctttttaacatctttttaaaaattttctatacaccttgaaaatgtgaatacaaagtaatgctgcatcatgtatattgccttattcacacatagcctcttatggtatatcatataaaaatggaacaatacagcaacaggttgaatgaacagtaatcaggtaacaggaaaatgagatgtctttaatatttcacttaaaaactcaatttcctaaagcatacatataaatatttggaagtatagttagaagaaaaatatctttaaaatattttaattgattagtcttatttataagataatttttaggaggctggttgcggtggctcacacctgtaatcccagcactttgggaggccgaggtgggcagatcatgaggtcaggaaatcgagaccatcctggctaacacggtgaaactccgtctctactaaaaatacaaaaattagccgtgcatggcagcgcatgcctgtaatcccagctactcgggaggctgaggcaggagaatcacttgaacctgggaggcggaggttgcagtgagccgagatcgcgccactgcactccagcctggtgacagagctagactccgtctcaataataatcataatcataataataatttttaggaagcatcagaaatatataagaaaaagattattttcttaattgctttactaaaaacacctctatgatttttcagtaaaacttgattcttatgtcatgtgtgagtgtgatctgcctctcttgggatactactgtactcatgaggagtgatttttttctccaacgacctctttgtcacgtcaacaggtcacaggaatagtgtaccctaaaaagccacctgccacatgctgctgaaaatgtaaaagtacacacatacacacacacacacacacacacacacacacacacacacaccaaaatcaggtatcacaagctgaaaataaaattgagtccaatttttttttaattgagcagttaatgtccttaaaacaaaatcctatactgcaacaaatacttagccagatcattctgatacctccaaactgtggtgtattccaagatacctctatgatctttgatttgatccacagcttttcagttatcatgcaaataccttcaagttttatctcatttctcagtgcaaactcattaaaaattttcagctgaattcaattttataaacatgttgtgaatgtcctctttatataagcaaggttgtaaggaactggccacataaacagaaaattgaataacatatggtttctggccttagtgatctcatgtgtgagttaggcatatgggcaaaatcagaacactatagagtataagtctaaaatggtagtattttataatagaggatgaagagggtgctgtgggatcataggtgacagatataactcccgttgtgggacttgagaaaggcttcacagtctggaaacatttagttgctattgaacacaaaataagactcactgttgagagaagggagagggagggcatttcaatcaaattaagattctgtggcatattcggaaactgatgtttttaaaaagagtaatgtttattacattcctctacataaattatatttctatgtaatatgaatgacaaatatttaacacaaaatgccttataacatttgaatgaaatccatcatatgacctgttatctatttccatttcctttttgctcatatcattatgaacaatgacctgataaattttttataagactttgctgaattagtaaaggattattaagtttagaatgaacaaagctgaccaatcattcaggcaaatttgaccgttttgttgtcgcttttcttatttctgaaaccatacaattccctgaaatgaataagtacatatttgataacttcctaaattaaggctcaaaacactggtaatctactgggctttcatttgttccttctatttgtctaatcctatctatatttctttatatgagctatgaaaatattagatttattaagttgtcctttatcttaatagagaagaatgtttttctatgacattaagaggaatttgatttttttctttaatgatctacttttaattttggtagagtagcattgataagatcaatattacacattgttaagtatgcattacatgttgataagataaatattacacttaaaatatgtttatcaaatgtatgaatgataaaaacgaattctgaaatgtatgggaaagatcttgaataaaggtctatgtacatttcaaggatgtctacatatgcaaattatcataatataataactattgaatatgattatcttcacatactttctttatttttcatctcttagatgaaattgggtattgttttcttatagctggaacaaagcattacagagaattcttagtgtgatttcattgaaactcactgttatatgagttcaacaaagtttaaattagtccatgacttaatcatcctttataaatcctatcactagtattcggtaaggacaaagtcaattaaaaaattagcaacagaagcattaaaagaaggattaataaatacaaaataagggatgtgatatctttacgtattgctgagatgttagtgctaaggaaaaacttccctgttcataatgtgaggtgggaaaaagaagaactattattgtatatttctcctctctaaaactgcctatctgactgtgtttttctgtgtcagccgtattaacagatgtttaattttactcactttagtatataaggcatcataatgtatgaactatttcaaaggccctatgatggctaattaaataaaaatatattaaatattagctggacaaaataaaatatgtattaattttggaaaaagtagatcaaggttttgcagatcttttcatatcaatatattcatttgctgaataagcttttattgtttaccaatattactagttttatagagatgtagatatcaccacagtatgactaattttatagggacacagatagatagatgttattttattccaatcttatttttacatataacaggtataaatatgcgcttgaaaggagtatatcacttaggagtcagtcagaaaagtaaagatcttctagtctaatacagtggttctcagccaggggtgattctgctgcacgctgagggataaattggcaatttctggagacatttttggttgtgacaattgcaggagtgttactggtattcatttggtagagacagagatattggtagacactgtacaggacacaggaaagtctcttacaacaaagaattattctgtccaaaatgtcagttgtggtgaggttgggaaacactggtctggaagaaggaatttactatgaggaactagttacgaaagtatagagacatttaacaagctgaacaaaggatagtgagatggctcagagattagcaactgtggcatgaagccactactacgtttaggtaaaaataagctaccatttattcttatagtaataataataataattattattattattatttgagatggagtttcgctctgttgcccgggttggagtacaatggtacaatctcgactcacttcaacctctgcctcccagattcaagcgattctcctgcctcagcctcctgaatagctgggattacaggtgtgcaccacccctcccagttaattttttgtatttttggtagaaacggggtttcaccatgttggtcaggctggtctcgaactcctgacctcagatgatccatccacctcagcctcccaaagtgctgggattacaggcatgagccaccacacctggcccactctttcttttttaattattgagaaatataaaaatatgtcaaaagtaacaggtgtggtggagttacagcatgcacataatgggatacagcccattatctaatctcagatggaaactagaaaaaaaagagaagatctttgctaaagcacagattatgtggaaaatcatttagaaaaatagcttatcacaacattaaaattaaatcctttagctgatcatttttccttgctattttttcttttaaaattgagaagacagtgagttttttttctttattgtcattatcttgatgtcaaaaaataatatgcacattataagtgggaaaaaagataagtcgaaatgaaatgaaacaatgcgaggaaaaaaatgtcacaacactcttcaattagaaaaaatgacccccatctttcctccaaatagaaatgacgtaactgaagtagtggaactttctcttccatggcaactctagagaaggggtagatggcatgggattgtggacagatggacacagaaagaggcctcatttattgttattgttaaaacttttacttctagtaatagtgacacctccttcagcatttctttatcaattgtcaatattttttggatcaccagcatcaccttctatatgtatgtctagaaacctcctgttatgaatttacacttctcagagtcaagacagaaatgctgtgaattgggcgataaataaaataccccccttttattgccttgctttgtctcttaaagaaagatgcctgttgggggactatgagaatgctttgtgcttctggacctcaagggacaaatctataataaaaattatgcatagtgatgagaaatatatataatgcaagtttgtagagatcagttaacttatcttgtctaggcaattatttctaaacaatgatttcaaatcattaactataatatagcccattcataccctccatttttgtcaaatccctgtcaccttcaaggacttggccatcccataggctgctctgcttttaatagaggaagatgctgtaactcttggtaccattgccagttatgaatttatccattaatgaacattgcatttaaggcataggtttatctccttctccaggtatgaacctgcaggattcctacctgaagcttaagggagaataaatccacctgggacaatcaaggacagatcaaccaatcagctcaaagcaggtgtgaattacacagtttatttgagtgacaaggtagctaaagcagggataataaaagaagggagtgggttgatgtggacagacgaactatggctttaggaaatttggtagggactgaaacatattttgtgtaatttatgtgggtctaatagcttttgaaacttgtttacaagacctgtgtaagtggtactggcatattcatgcatgagaaaacatcaagggaaaacttaatagttcaaggaggtgacaaagaagagaggaaccaattattttcactagccgtcaaaagcaagaaaataatcagcttgagcccttcggggaaaagataggttaaatattaagtaacagtttgttattattccaagtgttttcttaaagttgctcccatactttcctgttttctctgagggaatttagtttttttgttggtttttttttttttttttttataactgtcattggtcagagcttgatttgatgccagtcaaatttttttaaagagattatgaaaactgcttaaactcttccaaagggaagatgggtcattcttaacatgtgtttcaagaggaagagcataagagcattatatggtaaggctgaaagcagatatcagcgtttaggggccatgaagaggtagagctcacattggtaggatcattgactagaattccagagatcaaaattgtatgttagtctagcattggggaggacttgtagctagtatcttcattctagcttgggagcctaggaatcaggttaggcatcttgcacaggaatgggccgatgggctaaaatctccttgagagagatgattaatccaggacaaaccaagcagtcatgccaatgaattactttaacagggtacttcatatcctcatcctttgggcagcacggtcttcagagatggggcaggccccaggctgcagttgagattctataaactaaggtcaaaaagatgcagcagtgaagaagtcatgcttatcttgtataaatcatgttttcttttctttttaatgaaaatgtacatttaacacattttaaaactaaatattgaccctaaaattccaaccaaaaaatgctacataagtggtatttatttttgaatttccctcatgctcctcccactgtggggacaaggagtggtggtggaagagagatcttttagcaaacctgtgagtagagaattagaaggtaatgggaggaaggtaaaaggaaaacatcatagatggataggctcacaaacattaaaggccttcgtgcctgtccttcatgcctattcatccctctccagtatgtgaatcaatgtacttgttaaatattcattcacctcacatatttagcattaaccgtgtatcagggacgttgttagaccgttggtttacgatgatgtgtaaaatatcatttgtaactcagactaactggaagtgctcaatataataagatgtaatgttatggaacactaagtctgtgctgaagacttatctcctttaatcctaaaacaatcctggtgggtagtctcaatgatcatctccaagtcacagttgaggaaattaaggcttcaagaagttaagaaactggaccaacatcacaaaggtagcatcagagtgacagtttgatttcaaagtgtacttgacttcaaggcccacatttccttgcacgtttaatattgcctttctcaggtaaatataccattaaatgtgatacaactctaagcatttgaattacttacaacgtgcagagttaaaaccagcattatttacactatacttcagctcgtttataagtgaactattattttgtggactaacctatgaaatgtaaccacattgaattcctctgttaggtacaggtttggtgattccagggaatagagtatgactgaatgcacaggtaggggtgaagtgaacccggtcagaaaatttagagagcatcgagcagatcattaagcagctgtctttcaaatgtgcagaacacaactcatttgtaatctagggactatctgtattgattcttcccagggaagttacttatttttatacatatgtggtgtgttctgtccataataccattctacatggtaatgctcaactttattatttaaaaaaactgctaataatgaggtttttctttgtatcacagaagcagcaggagcaagttttctttttccttcccagtttttttaagtactgccaaggaatgtgattttgtcagacttgtatttcctattaagccaatctgcatgactgttccttctactagctttacctgttcactcatttattaattcatcaaatatttgtagagtgactattgtgtgccacatactaatataggcacaaggataaccaaaaacagacaaacgctgtcctttcaaggagctcatatagtaatgggaagttaggaaaggagaaaataaatatgtggtatttcaaatggaagtattaaagtgttaagaagaaaagagaaactaacaagatagggaaaaagtgacaggaacatgatgttttattttttatttatatatattttttgagacagggtctcattctgttgcctaagctggtgtgcagtgacgtgatcatggctcactgcagccttgacctccctgggctcagatgatcctcccacatcagcctcccaagtagccaggtctacaggcatgtaccacgatacccagctaacacgttttcttttcttatagagacagagtctcactgtgttgcccaggctgttcttgaactccggggctcaagcagtccacccacatctacctcctaaggtgctggaattacaggcatgaaccaccatgcccagccgaaattgatgttttatatatggcagtctgggcagacctctttgatgtgatatttgaacagaaatctcaagagagggagtgtattagcccgttttcataccgctagaaagaactgcccgagattgggtaatttataaaggaaagaggtttaattgactcacagttcaatatggctggggaggcctcaggaaacttaaaatcatggcagaaaatgaaggggaagcgaggcaccttcttcacaaggtggcaggaaggagaagtactgaggaaagggggaagagacccttataaaaccatcagattttgggagaattcactcactatcatgagaacagcatgggggaagccaaccccatgattcaattacctccacatagcctctcctttgacacctggggattatggggattataaggattacaattcaagatgagatttgggtggggacacaaagcccaaacatatcattttgctcctggcccctcccaaatctcatgtccctttcacatttcaaaaccaatcatgccttgacaacagtactccaaagtattaattcatttcagcattaacccaaaagtccaagtccaaagtctcatctgagacaaggcaagtctgttctgcctgtgagcctgtaaaatcaaaagcaagttagttacttcctagataaaatggaagcacaggcactgggtaaatatacccattacaaatgggagaaattagccaaaatgaaggggctacaggccccaagccagtccaaaatctatcagggcagtcaaatcttacagctctgaagttgtctcctttgactccatttctcacatccaggtaacactgatgcaagaggtgggttcccatggtcttggtaagctccacccctgtgggtttgcagggtagagcccctctcctggctgcttttacaggctggcattgagtgtctgcagcttttccaggcacgtggtgcaagctgttgatcgctctaccattgtggggtctggtggacagtggccctcttctcatagctccgctaggcagtgccccagtggggactctgtgttggggctccaaccccacatttcccttccacactgtcctagccgaggttctccatgaggtcttcattcctgcagcagacttctgcctggacatccaggagtttccatacatcctctgaaatctaggcagaggttcccaaacttcaattcttgaattctgtgtatccacagactcaacaccacgtggcagttgccaaagcttgggacttgctccctctgaagcaatggtccgaactgtaccttggccccttttatccatggctggagtggctgggacacaaggcaccaagtcctgatgccgcacacagtggtggggttgggggggggacctggtccacgaaaccatttttgcctcctagacctctgggtctgtgatgggaggagccgcaatgaaggtctctgacttgccctggagacattttccccattgtcttgcctattaacattgggctccttgttaaatatgcaaatttctacagccagcctctccagaaaatgggtttttcttttctactgcattgtcaggttgcaaatttttcaaacttttatgctctgtgacctcttgaatgctttgctgcttagaaatttcttctgtcagataccttaaatcatctctcaagttcaaagttccacagatctctaggtcagggtcaaaatgatgccagtctctttgttagtcatagcaagaatgacctttactccagttaccaataagttcttcatctccatctgagaccacctctgcctggacttcagtgttcgtatcactatcagcattttggtcaaaaccattcaacaagtctctaggaagttccaaacttttccacattttcctgtcttcttctgagcctcctaactgttccaacccctgcctattacccagttctaaagttgcttccacattttcaagtatctttatagcagtacctcactacctcagtaccactggtcttaactcctgcgctcaagcgatctgcttgcctccacccctaaagtgctgaaattacagacatggtccattgtgccgagccaaaattgatattttatgtatgacactctgggcagacctctatgaggtgacatttgaacagaaatctcaaggaaggggagaaattatccatttacatatttggggaaagagcattccaggtagaagaaacagaaaatccgtagtcttgaggaatgccgtgtatatgcagtatttttcaaacttgttattttgaaatacatatacacttacaggaagttgcaaaagtattaagaaagatcatgagtacccttcactcatcttcagctaatggttacatcttacataattatatgtaatatcaaagccaggaaaccaggaaattgatgttgatacaatctatgctttattcagatctcacatcttacatagctatgcacaatataaaaaccaggaaattgatattaacacaatctatgccttattcagatctcaccagcttttacatgcacttatctgtgtctgtcattctatgcaattttataccatgtttagagtcatataacaactacccctattttgatacatggtactgaatagttccagcgtcacaaaggaactatctcaagccaccctttaattgtcacacccatccaatctcccattctacttcctgaatcactagcaacccctaatctgttctccatctctatgattttgtcttttcaagggagttttctaagtaaactcatttggggaaagaaaggagatgaattgttctagccacggagtggagaacagagagtaagagtacctattgaagcagagggagtcattgcaataattcaaatgagaaataatggtgattctaaaccaggaagctttcagtgaaaacaatgagaggtacatggattctgggtatttttggaaggtagcactaccaggtttgctgatgaatggggtatggggtgggaaagaaagagaagagcccaggatgagtccaaggtggataaggtgaatagaattgagaaaatggtagaaggatcaagttagatggtagaggggtaaaggtggaagcaataattttgttttggaattgttaggtttgaaatcttgttagacatcccagtaaagtcacaaagagtgcagttggatgaaagtatgggattcagggaagaagtatgtgctagagatgcagatttgagagtcatctgtgtggaggtattattcaaattcaagtccccttggaatgaatggctattcaggcagggtcttcataaaaatgcttgttgcatgcctgtaatcccagcactttgggagtctgaggtgggtggaacacttgaggtcaggagtttgagaccagcctgatcaacttggtgaacccccatctctactaaaaatacaaaaaaaaaaaaagttagctgggcgttgtggcacatgcctgtaatcccaggtacttgggaggctgaggcaggagaattgagccaagattgtgccattgcattccagcctgggcaacaagagcaaaactccgcctcaaaaaaaaaaaaaaaaaaaaaaaaaagcttgttgcttcaaattcatgtcagtctgtaaaattatctgggaaggcagtacaaaaactgtcactttgactacgatgtttctggtgacccatcttcattgatcagtatggaaaaggcatgtctctgaaaatctctgagagtctttgatacagcaagaacataaggataaatcattcttctatgttcatggttgtagaggatcttgaatgtttaatggcagaatagccagatcacactctggcacttctgtatgagaggctgagggatgttactgattcaccccgagaaatatttactactaaggggacagaggcaaaggggatacaagacttcaccctgagctgtagcgctccctccttccctatcctgctttcattcttcacattgttttccttctttcttttttattattatactttaagttctgggatacacgtgcagaatgtacaggtttgttacataggtatacatttgccacggtggtttgctgcacccatcaacccgtcatctaggttttaagccccacatgcattaggtatttgtcctaatgctctccctcaccttttccctgtgtccacattgttttctttctttttgaagcctctcattcactaggtttcaatcctgccttgctagtgttctaactctaaggcctaggcaagttatttcaccgaacttagcctcagtgtcctcatctgcaaaatggatagttttatgatatcttcagcccttaaagtcaatggttctgacagctagggtgtactatcttcttggatatcagtcatctcaagcaagccctccttttttggaccttcttttcacacacttcacataccttagagaacataatacacatcctctttactcagggcttattctttataacaggcttcctaattcaattaactcaacttttcaaaaatattagtgactactgtgatgtaaataaatttgcattttataggggtcttagtaacccagaagggagtggggaaaattaatatatattgagagtttattaagtgctaggtactgtaaatattttcttgtatttaatcctccgagtaattctacaacaaagatattatcattgctattatgtaaataaaagaacaaagtagaaagaaacccacggtcttgtataagctcccctagttggtgggtattgaagggagtatttcaatctttggtagcttctgagtttttgttctctcagggaatctgccagatgtccagggcacctgccaaaccctatgaggctataagaaaaccattaagggtcttagattacccagctttttgggagttagaattctgaatgaaatttagtgttcctgcagctacaaaggaattgagttagggaagtgatgactttatctttagctacattggttattttccttataataatcctggcttggtagattagaggcagcccgagtaacccagaatcgctaaaatagaagtgcgagctcattgcccgctgtccttcactatgtttgcatataggaagcaagaataaaacaagcataaaataggctaactagcttgtcagagctcttcacaccaagtctttgtgagttccaataagacactgactattattaaaaagacagagactccacataagtaggaatttattgttttccttttcagtcaccaaaggacaatcctctgcataggttagcaaaaaatggtactgatcctataatctctaatattaaagtttagatttggcaagctgtacatcttatgttgttcattaacaaaaaacaatattgattggtatcttgtactataacttgtactgtgggtcaaattccaatacagcaaataccattgcaataacaattctacaaaactacatcaaaaaaacctttcatgtttgagccaacagcctgatagtgctaaggactttgagtacagtatgctagaagattcttaacagttatttgtcctggacaacaaaggttgactccattaaaaacatagccatcagtgtgggattatttccaaatcaagcttttggaaaagtcaaatgaaagtttgcaagcaggtggggcatggtggttcatgcctgtaatctcagcactttgggatgctgaggcaggcggatcacctgaggtcaggagttcgagaccagcctggccaacgtggtaaaacccccatctctactaaaaatacaaaaattagctggcttttgtggtgcatgcttgtaatcccagctactcaggagcctgaggcacgagaatcacttgaactcgggaggcagaggttgcagtgagccgggatcatgccactgcactccagcccacatgacagagtgagaccctgtcttcaaaaaagcaaaaaacaaacacgcaaacaaaaaaaaaaaaaaccaaagttggaatgcaataaatgttcattgaatgaatactgaatagggagtttcagctaatccactcaaaatagtgctgaatttccagctctaaggtcaatgcttggcatatatatcctgaaggaatgaatggacacagagtaattttttttctaaaatgcaaattcaattatgtcacttcccttcttaaaatccttcagtagcttcccgtagcctccagcatattattttgaatagtgcttctcaaactttgatgtgcatcagaatcacctggggattttcttaattaactgatgctgattcagtaggtctggggtattgtctgagattctgcatttctagcaagtgctcagggttatagcaatgattttggcctgcagaccatactttgggtagcaaagacataagccacttaacttgacataaaagactgtttagacccttagtttctctctcgctctttccccattttgagcttttgctccggttcatgtttttccctgaaaataccgtgatcttacattgtctgtctggatgctgaattttccctaattctgggcctccatgtagttttaggtttgacatcacaaccaccaaaagatttccccttctcccttaatcttggttaatgtcactctcatgtattatactgttaatgaagcattgaggacataaaacttatcaaatattttatcacaatcaatgatggcaccagtgataacatccaaatgcctgggtgagtaaataagaggagaataggggacttgttgttaaactaagtttgcagagaaaaaatgtactgattataattaaattggatgtttatttgttatgacaaaaaaggagctagagtcttttaatccaccccttggcaccactgcttatctccttgtaacatacgtttgattcccatgtctatttcttccatatgggaaatttcagctccctaaacatcaccaatacaacctgttgataagacaaagttaaatttattgcttactatggtaagaaagaccacagcctggacaaagctttggtagtatttcataaggagaaaggtgaggttggatttcattgggagtatgaagcttggtttaagattggtctttcactgtgggggcacaattaggattgggtaaggatcatggtattacaacttagtttggtggaaacagcacagtgaagatttctagccaagaggctcagagactattaaggtgtgaactctattgatgttttttgttgaagagttgatgggagtttggggaagttactttagtgaacagtcaaattatttgcctggccaagagttatctgtaataggaaagttatgctaatgaagacaatggaaaggtaaaccatgttaatgtcgacagccagctatgtgagcataaggggtaggtagctttggtcctccatgtccaaactgtttgtagtggtaagtgatcttcattctcacatagattgaaagcttcctgaggacagggcaatgtctttgtaaactttaaaatatctatgtcctgcacatcacctgccgtagacaagcatctagtaattgacggttgggtagatactgagggaaaacatgcaccaaataaaaatggcaataggacacaaattcactatcatttggaagaataacagtgttttccactgatatttgctacacacagtggggtccacagagcagcagtaccacttgggagcttattggaaatggagactctcaggcaccaccgcaggtccaatgaattaaactctgctttttttaaggtcatttgtattcaattattatttttttcttttttctttactttcgatgcatttttctttatttgtttttgagatggggtcttgctattttgccgagtctggtcacaaactcctgagctcaaatgatcctcccacctcagcctcctaagtagctgggatcacagatgtgagccaccacacctggcttgtatcacattaaattttgaggagcagtgctttaatatctattccattctcatcacttgatgaggtattattaattccacttatggatgtggaagttgaagccagaaagtttaaatgacttgtacaaggtcaaacagcttacaggtagttgagccaagaggctctcaagtcttctgcctccacaaacccctgttcagctgctgccctacaatggaataaaatatactaatcccagagggacaaatatgctaaaaatctcaatattatacactttggaaggtgcaggtgcattatctttcaattctaatttctctttcaagttttctgatgcataaaaatatgaacagcaggtctgagcaatgtttagatgccgtgctttgatccttttgccattcaagatgtttgatttgcattctgccaaggaatgtctggtaacctccatgatgcagaccacaccattagtcaagagagagctgacgtaccttcatctgagagctggctggctgtgagctgctcagagggaaaggatttctatttacaaattgtatcgattatttataaataaaagttccccttgctttcttcagttgtaaaatctgcagttagagagtcgggaagaagatcaaaactgcatacatttgcatctgccaagcctgataactagttccagaattacagaaatggtgctgaaatagcacctcaagtaccaggctctatcaaatttaatctatccataaggcaactgccaattatattttagagaaaaaatgtagactgaaaagatagacaatccaagtagcaactcctgtaaaattatatgcccataggagcaatcttgaagatataaatattggtatgtttctccttcatttatcatttatctgatcatttgacaagtatttattgaatgcctgttaagggtgtagatatatgtggtgaggctgcaggtgtaagtaggtctttctgaggatatgcatgaagttgatgttcataacttggagatgtgtgtatacagactgaggattccttcagtggatattaagaagtggagtaataggcagtaaagaatacactagtcagttgtggtacataaacacgtcagcaccacttaggtattaacttcctgttttgttttgtgtgtgcttaattacgctgtttattaaacaagcacatcataatctgcagatattgtcataaacagcacaataaagcctgccacatcagaatgtcatctatcaaattaggtgtgttcctcagctgtcccgataggcacacacctgtgcctgtaaataggcgcttggcggagattgcttccaggtgtggatctgttgggcgaccttgggatgtagggcactttggaaccttttcctctagcttcaggaattaacctctgggcttggttccatgccagcttgcattttgctttgggacagtaacatgtaaagaatatgcctgtgaatttagggttactgagaagtcctcatagaagaagtaaaatttccttgaggaatgggagtcttttattcaatccaggtttaatgcaaggcttggtgaacagctccagaaggttaataattgcgtgcgtgtgtgtgtgtgtgtgtgtgtgtgtgtgtgtgtgtatccttttgtcattcaaaagtatacgtatacacacacacctgtacagctgatgataaatatacattgtatcaatgagttcaaatgaagtgtgctattcattcactgaggaatgggctattataatgaactattatgatattagaaattgtcagggcaataagcaaataatacatacggttttcaacaaactttctaagtattgttatcagtgggtttgcttaaatctttttttacaaatttatttatttttttgagacgaagtctcgctctgtcgccaggctggagtgcagtggtgcaatctcggctcactgcaaccactgcctcccgggttcaaaagattctcctacctcagcctcccgagtagctgagattacaggtgtgcgtcaccatgcccatctaatttttgtatttttagtagagacgggttttcaccatgttggccaggacagtctcgatctcttgaccttgtgatccatctgcctcagcctcccaaagtgctgggtttacaggcgtgagccaccgtgcccaggcaatagccccattgctcagtgaatgaatagcacactttattttaactcattgatataatgtatatttatcatcagctatacaggtgtgtgtgtgtgtgtgtgtgtgtgtgtgtgtgtgtgtgtgtgttgaatgacaaaaggatacacacacacactcttattaaccctctggagctgttcagcaaaccttgcattttttactttcattacagtgtgtaaataatttagcaaattctaatttgaacctgatatcaattgagcatttaatatttagccaaatatttatcaagtgctgactgtgttctagatgctggggctgcaatttcgaaacagaccattgaggccctcatggagctcacaataaatgatcttccttaaagtatcaggtctctggtttgttaccgtattttttaaattgttaaggaaagaaaaaggccctatctttttgtagacaaacatgccctaagtgcttccagaaataatctccatcaggtaatgcagactgtgtgtggagtgaaattgagtccaatccatgatccagcagagtttcagcccaggatttctttagagcctttgctacacacaaagttggctgatgtgccattcagcatcccagcagctctttctcttcacactagcaatggcaaagctttgtgcggaggcattgctggctgctctgaactaaaagcatccgtggggaccgaaagaggtttttgcacaccttattaaggtaggcaagtgtgtctgagtgtgtgtgtgcctaaaagctggaagacatctgttgagaggaaagtgctcttctgtgggtctggcagcttttctgtaagtcttctattctgatgcaggagcgtgtgagcagtgggtgggaggagatgctttggtacttggaatgctgaggtccggattaagtggtattgtaatagctagttagaggcagaataaaaagctgggaatcaaagcatttaaaaatgcatccttccattatttgctctcaagttaaaccatattcattctaggggaaattaaaaaaaaaaaaaaaacacagcaagggcaagtagcccaaatctgtaaggtctttgagcttctctgttcgtccagcttttgaagtcttcctacagccaatttgtttggctcctctggagggggcaattcatatccacttccctctcctggagcatttctttcttctatactccatcagggaacaatagagtttaacagtaacaggcaattttttttttttttcaaagcttgtgccctcttctgcgtttaaaggtgttttttaagagactcctgctaggggaatcttggcgcctgtgtgttaagacggcaattaacttttagtatcagtgcttacattaaattttctctctttctgctttactaaagcagtcattaaaattcagtgtgagtaccatgaaactttatcataaaaccctgctttgcttagagaaccttgattgttttctgaaagcagccttctcagtttatatatacatagctgccttccttggaatatcaaattgctttgtgtcacattaagaaacactaggttgaacctctatactgtgttttatctgagaaaaatactactgcaaaaagtttgatttgttcaagttttaggatgaaaatttctttgtaacaagttatttgagttgcatactatgtcatcgtatatctctttagttcaagtaattttgcaattaacatacggttatgtaaagaagataatgatttattttttatttatatttttaaaagttattaagtgaggttttcctttcagtaagagtttagaaaaaatagccagaacaagtaactggacttggaagataaagatacctttgcacttctaaattttacctttgtacacttcggttgtgatttaatcattgaaatgcctctgctttgaagtaaatgcatcacttatggtgtatgctgtgttttaataaagggaaaacagttatgggttctctgttgcacatttgaatgttgttattttttgctgtatttaataacctcttttttctcttgtgaggtttactttggaaatgaggcatgttcaaaaataggctgacattcagcttctatgttttaaatttaaatgctgtctgtgttttatcacatctggaatgtgtggggagaaaagataccaagttttattatttagatttaattgtagaattgcagattgatatttttcaatgcattttcattatagtttctgccatggaggcagcgtgagggctttcaggaagatggagtggtgtaattaccaggtgcgcacgttcattaatccttcctggctagagaaagcttcaagttcttctccagtggcccattcgtaaagctataaatatctaaattgtgtcagccaagaagtcacacagaatggtggctctttttgagttcaatttcatgcactgttgctttggtcttgtgaggaaagctctgaattccttaggatagtcttggttgtgaagttccaaaaacaaaatatcaaatcattaaggatttaatttaaaatacatactcttctttcacaaactagatgattgcagtaatgtggattataaatttttttttttgctttatttctttagagctcctctttttattttgtatgatcaagattatagctgagattttggtgatttttttaaaaagatttatggcttatggtccatcagtctctccactacttcaaacctgtgtacccctgtatattatctgcagtactggaatgtttgcattgtatgtggaagctatatacgatttggtaaaaaataacacttaaaggtcttcgctaagagtgcttatttaatcattaaatatcccttaataaaaataattccagagatattgtctgtgtacaaacttaaaaaaagagaaatataaaatactgtgatgtgaataaaatgtatagcaatacactccaataataccattcttatgttttcccttgttctcaactgaaataactaagctaatagagacgtcagtaaggaatgtgttgtttcttcataatacaactacaaactcatctgataagaacaacctgagagtgaacgttaactttcctcattagaaagattcaatttaacacatatatacaaatacatttttaagataatgatatttgcagagtttttgtattctatggagtaaaggagaattatcacatattcaaagtaaaggtataaaatacatcttaatgttttacttaaattttaaagggtccaaaatatactaaaattgtttttctaattctttcctatgtttaaacgtgccagagtcattggaaataggacattctttttcttaagaagattttgcccaaaatatttaaaactattttcttttcccttgattttacaatttcaatattcatggatttttctactttaaaaataacagtagtttttatgatcttaaaacaaatgtttaagggcactttcgctctctggagactataccatccacatatttattatcagcaaaagaaagggcagggcatacttttatttgaagttgagtataaaaatgtgtctgtgtgtgagtgttattaaaaagataagtgaagagacaaatatagaatccaggaacattttcagcctggcttttactctctctaaaaatctaatgaaacccttgagcatctcttatctcaaggtacattaggaactgtccaacactatgatccgatgggagatcagtatattcatataaagaagaaaatttgttgttagtgaaagtcaagtcttttaaaaaaataatagttacagcatttgcaatatacaagcataatagatttactcaacgcccaccccccatctttaaaaaatcaatttccgacagttgtctactttaaaattgaacatatttgctacctggagggaacattgtaatgtagcccatatgtggtatgcatcctgaagaaaacctgaaattatagaggaagttatcctgccttctttcttctgttgaatgagttaaaatatattaacaatttgcctttcactttgtatttatcattttgtatctttgcatatttacatatacattcatgtgtacaagggcatatatactcacaggtcagggctatttaaacagctatttatttgaatatgccagggaaaatctccaagatataaagaagcagttattagatactatgtcagtatagaattaacagccatcttttttaagatggaagagaaaattaattaattacatacaatttctaacctcaagacattttctttctggagacaaggaatactgaggtgctcacgatagtgaagactcaacaagaccctaataaaatagatgaggataagtaaaactacaatagccaataaaaaacaaaaaacaataaaccatgtttcgctggcatgttggtgagtatctctgtaatatctgtcaataagggtctctgtagatttggagtaatgttcaggaactacctgtactagagaagacagtggagaggactccagtggctaaattctgctgcctttgcttccagaaatgtaaataataaggaggtattgtggcatttcctggaagcagtagtcttgtttcatggtctgactgtataagaatgcctagagaaacataacctcagctgactaaactcccttgatgattgtcactttgtcactgaactctgaccataccttttgcctccagaggcaaaagacgggtgaggaagtgatctcctcatctggtttttaaacaagtatataactagagaactggattatctcctaaacccactcttgtccctggaaaaaggggagtcatcctatccgtttcttagccaatttatgtatactcttagtttgagagcatgagaaggaaaactattttcttttcttaccttggctgggtttttaagaatttatttttagtttaatcaaaataatattttaaaaggtagtaagcctctcataagcagtttgatctgttctaaaataacttcaatttttctttttttaaactttcttttatcttacacacaaagtataatagtaatatgtactcactagaacaaatgaaacaggatggagtcacatagagaaatatatcatattctccctatcccctcccttaatattaacatttaggtgtcatgtgcttctccattaattttcattgcaaaggcctaaattttcttccaagagtgaggagtagcagcacggtagtttggacctgatatagctctctttccctagccttttgcttaagtgctttcctaggggctgactttacttacctaaagatgtttcaagcaagggctcacatttttggtagcagaagacacttactgattgctctcactaataattttgaaaggaatgtcaaaatctgggaggatcatgaaagaaatatcagaaatttcctttcagctgccattctccttaatactgttatcaataaattcagcatctcatatgtgatagcaaaaaaggtgctgccttttgttcttgcatcctgaggttcttacctaataccatggtagcaataaagatggtgagaaaattgcttcttctatggtgttcaggtcctgaacgagcaccctcacctccacagacggtggcaggtattcaagcattttacagactttggagttaaatatagcagtgttattctaatttaggtatgccaccaccagcggcaccggcaactgcaataggaaaaatgattggcaatgccagctatctgatgttttcatgtgccaggtgctgtcagttcttcacagtattacattccatcctcacaacaagagagtgccagtgagtgttgctgtgtgccagtgcccaggctaagggctttgaacacattaccctgttttatcctcataactttccacgttatttttattcctgaatgaagaaacaagttctctgtagagatgctgtcattgatccactcatatcctttcacatccgtttaacattttccctgctgtgcttttactcccaacaactagctccctaatcgctctgttggagggtggccttgaggctgccagagcctatttggtctgtgtaaagagagagatggatctatcctggaatttatgtccctgtgtgtgggaagcccttaatcaatgactgctggttgcagacacataaatacgtgagctttcttgttcccaactgagaaattcagaagtgtgaatggcactgccaccctgggcttttatgccatatatgtgtttggtctgtttcccttcccaatctcacttcattttcccttaccagtgtttcttgaaaacacatcccattagatcatttttgcatgaagcttcatctcagaacctccatttagggaacccaaactaagatattctctaaaatagaaactttattgataaagtttccaaactgtcttagtagatggccaatataagaccaagccaaatctttctgggtccaaattccctgtctttaattaatagactccattacaacacattcttcaatctttagtcagcaaacacttaccacgtgcctattttatggcatattatatttataccatagttaggatattatggttcatgaatattttatatctgtacacctgaaattctattgacctctctgggccacagttttgcatctgtaaaatcagcacaataatgctacttatctcatagagtagacttaaaaacgaatgaaatgatatatgccaagtgttgagaatcacaattggcaattactcatgctcattaaatattagctgtttttatgagtattgtttcattttcggtgcataatatcctatgcaaagaacaaaaggtattggtataggcattgaaacttgaagcatagaagaaaaagttaattaaccggtgccccactagatgcctctaactgctggctccgtgtatccctttagccttggctcgtcacgagaaaaccttggagacatttctgctggactcagcagatcaatttaagaaagatgaatgacatttttcttgaaatgtattcagtcatagctgcctttttctactttcatattttggagttcttagaaaaaattaaggactcctttttttaaagaaaatggtataaaagaaaatgcatatcactttgtcactttattattgtaacctcatcaaagtattcagtgtaaagacagtagccaagtgaactcttcttgtaatgctcggaaaccattttagcaatggtaaaattgctgcaatttatattcgtcaaattgcatgatttgacttattttagaaaagttattaacttctgaagagaatgcttcagaagcatttaaatgagtacaagttatcaccagtgatatacataaatttcatttcaaaatatacttctagaaactgtacttagttagctatagtatttgtacaaggattaattcctatttcattttgtaggaatttatttatgaatgtctatggcctgccagtgtaaagcagacttagagcatcatcttttacaataatctttttttttttaatcaaaggggagatattctggtaaaacaaaacaaaacaaaaacaatagtttattctgcatttttattaagtccctctgtaagtcatccctgaaatgggatatgtagagtcttatatttatttatttctcagaagcttattggaggtgatatgaaggattttaagaccctactaactaacaaaacaacaatttaaaattaattttcaaaataccttaacaaatcttattctccttattttcaaattctttaacaatgtttttcttattactaacataatatcttctgatgtagtcataataatatctaaaatgacaggtctaagtaacttacatggattaattgagtcttctaaatagtaaggtagatggcactattacttctatatgagaaatgaggaagtagaggtataaataagaaattttttggccgggtgcggtggctcacgcctgtaatcccagcactttgggaggccgaggcgggctgatcacgaggtcaggagatcgagaccatcctggcgaacacggtgaaaccccgtctctactaaaaatataaaaaattagcctggcgtggtagtgggtgcctgtagtcccagctactcgggaggctgaggcaggagaatggcgtgaacccgggaggtggaggttgcagtgagccgagatcgcgccactgcactccagcctgggtgacagagcgagactccatctcaaaaaaaaaaaaaaaaaagaagaaatttttttgagtgtatacagttagaaaatggcaaaatgggaattcagacccaaacagtaagactcaaggatacctttcttatcagtatgctaatatgaaaacctaagcatactagaaaatctaagtgccagttggaaaccagaattaacattttggtgtgtaactttctggctgctttttctatgctaacaaacatatatgacatacaaaaatacacacatacacaaattcctgttcactacttcttttatgttaacatcacaatgtaccgtacacagctgtattattttatatttgatttcatattttttctaaagtcagtgtatttgtcaaatatcaacttatctatttaataggaatatgggatgatctttgcttatacatacatacatatgtatataaaaacaaaatcaagtattttaagcgttcaccagaagtcatatgtcaatcagtaaagtatataattttttgctgccaatgacatatatcataaaaacgctacctatcatagaatgaaaatgaaacacagcaatattgggacacctattctcaagcaacagctttgtgatttattagctatctcacatgaaataactcattaacttggtattccaagcagcaaaagaaggatcacttaggtcacttgcaaaataatacaaagctaggtttaggggtgggttgcgcttggtgggatgtagatgaaaccatatgggcccttgagtttataattgctgggatctgcatggtgggtatatggatgtttattacagtatgctagtgagttaagaaagaagaggaattattattgacttacatcatagagtttatgcaaaaattaaacgataatttatttttaaactctagaggtataggtaccatcatgaagggacccacagaactgatgtagccagtaattattggagctggaacagatactctgctgtcagttgttctggttttgtggtcattgttcttgcctttgcaagttaccaactctaagaccttgggcaatactttaagtcttggttgtctcatctgtaaaatggggagagcagtaagtgtcttaaaggtttattctcatgttatatgacttacggtatgtaaaacatctgcgtttagacacatagagggtgcttaatggatgattgctctcattattaggctacatctaatctatgaatttaaaaactgtatagaaatatgtgacagattctttaagagccaaataccaactacagtgaaaaatacttaacacttgctgagctcttagtatgtgtcaggcttaactaccttaatgctcatagcaatcctataagataggtactcttgttatcctattttatatcttctaaaattgaagcaagggaagttaaataataggacaaagatcatacgctatctatccatatatacccatctggctgtctacctgtctccttccatccatccatccacttattcatctacccatccatccactcagttacttctctctctcccaccatccctttccctttccctctccctctccctgtctctgtcactctcctttacttatctatctatcgatggatcggtttatctatcatctatctatctctatcatctatgtatagttgttaataacactaacattttataaattacaagactgaaaaatgttttcattaacttatggtaacaaaagaccacattgtgaataaaaaaagcagtaaacacaggtctctgcacatatgaaagagatgtcctaaacaggaagagatgtcctaaacagtagggatacatagtatcatacaatcaaaacatggcagccctataaaacttacaaagcaatttcatgtaagttatttcatttgactcttaccacaatctatgaggttactatttttatttttctcattttacaggttaaatttaatatggcttccaataaaaaattagtatggttaataaatatcttgacgtcttgctcctataatcctaccgatagtttacagtaattagtaaaataaaataataggaaaaatacctttgatactagtattaaattataatcatatcattaggtaatttcaatttgtgattttcaagaatctgtaatatggtagcttcttcctactgacatgtttgaattcattttaaggcttataattcacaagtaatctatatattatctaaaatgtaaatgcacattcacatggagataataaattagcgtgaaatggctgtattttgctctctataatttttaacatacaggaaatcactgttgtctcaaaaatcaaggaaatatagtatttgaggtgaacttattctttctactattaacacattttaatatagttctctcacagtgcaacagagcaagaagctttcagacacatttgctgctgcaaggagcatgctgtgctgaacttaaaacaccttccctttcaaactccttgggactgtttttttccaagagacttcaaatgcactaaatttagcatccgttggaggcacacccaggcatattatagtgaaagccccaataactgaatgtgttaccactattcacaatgtttatgtgtgtatatgccttatctatgatgtattgcaaattacaaaaattgtgttattattcacagtaacaaaaacacttccagcaaatttctaacagtgatctcttttgaaataacttacatacatgtgtcatgggtcttaaactttgtcacttttatgtttccatcatgttgttttagccagtgagggttttgtttggttttcatttatgattatatactttcaaaaaatagatttcaaagtgtgaatttgattgattgattgactgattcattgagacggtgtttcactcttgttgcccaggctggggtgcaatggtgcgatctcggctcaccacaacctctaccacccaggttcaagcgattctcctgcctcagcctccctagtagctgggattacagatgtgcaccaccacgcctggctaattttttgtatttttagtagagacaggggttcaccatgttggccaggctggtctcgaactcctgacctcaggtgatccaccctcctcagcttcccaaagcactggaattccagacgtgagccaccgcgcccagcccgtgaattttatttttgaaagacaagaatgtccttgcctaattgcataatagtttaacatcatgaagactaaatatgctttttagccatgacaattttatttattattgttttcatttttaattttctcaaagatcctcatcagtgtactctttttggtcttccttataagcgtattttaacaggacataataataagataaatcccaactttttaaagttgtatccgtatgtattactttaaagtgctattaatataaacgaattagaggcaacttttattcaatcagattttaagtaattttaccaaaaatatggccttgataatgtctctgtaacaggttctctgtaatatacatgctgaggattggtttgtctttgcttttgatactattttaattagaaaagtaatggggaatccagacccttctcatttaataatccagagaaaaatcagtccatgttctaatagtttaaatttttctactaaaacccatgtgagaatccatatgagtggaatggagaggagttcagcttcaaagttggcagatttgagatgattctatggcaacagaaatgtgcttgagggaaatcagttgcggcatcttctataattgtgtcacctagattttgccttaggaatttctagatttccatagaacattgtgacctcaaatgctttatcttaataaagaaataaaagcagattagaagaattatttgcctacagtttgtgggagatgggcaagtcttaagagtttattaggtacccagaacgaaacatattttcttgggcctcataatcacattgaaatacaaggatttagttatacacagtgaccagttagtgaatgacagtcttcagtatctagtagacagtaaacatataaagatgtatttgtggccgggcacggtggctcacgcctgtaatcccagcactttgggaggccgaggcgggcggatcacgaggtcaggagaccgagaccatcctggctaacacggtgaaacctcgtctctactaaaaaatacaaaaaaaaaaaaattagccatgcgtggtggcgggcgcctgtggtcccagctactcgggaggttaaggcaggagaatggcgtgaacccgggaggcggagcttgcagtgagcagaggtcaggccactgcactccagcctgggcgacagagggagactccatctcaaaaagaaaaaaaaaaaaaatgtatttttacttttaactacagcgagagaccctggcagcctacagcatacaattagtgttcattatttagattgcatggatttaatgtgaggggtcaattacttgtctaaccagtgagcctagcctcttgctcaatactgcctgcttcatgagggtgaactgtgctggagaaatatattacaggattatctgcagattttttttaaatgagtggttaagtcaaaagttcttgtgaaaattcagagtaataaattattatgaagttgtgtaactaggtaaaggatagtttcttttacacgggtaaagattaacatgaggaggaaaactttagcaatggcatttaattccattcaatatatttatattgagctcctttaaaaatacagggccttgtggtgggtgctgaggacagaacaaaaaccaagtaatacatgaacataacccttgatttcatgatctagtagacctataaaagttgtcgatatctgatgaaaagaaaatggtaaagatattccaaacagtgtatgcaaatccagagataggatggaggggctctacctgaaggatgatgataagaaaaccgtgttgagtgaagggtgatttgtggaattcagataaaatatcagtcttgaatgctgagtgaatactcaatgattgactagatcccatggacagtaatttcttcaattatgacgatgctagtgtttatgactataactatcattctccatgccaggcactttgccatttggtaaatgtatagtgtgctattctaacaagcatgcacagagcttttactttaatgtatccatgagtttattggggttcagaatttaggtaagctttgcaaggtcgtagcatggagtaaaatatctgaaattcagacccatatctaactaagttcaaagactgtacagatatttctcctcctttgtgcagagaaggataggaatggttccatattatcatggacttagtcagatgttttaaaattataatgtcctgtgttaatgaagaagggatgatattcagtgcatattcttaaccgttactttgcttaatgctctcgacttttctgtgagatggatagtgtagataaaatccccaaggggactcagcaagtgcaagtaaaacaatgaaactttaaagccctttgtcaaaacctctctttttctcagaggatggaagggccgtaaaggttggtgaggaaggatggaccatttcctatgtagtcttctgacaatattcaaacaaaaggagagtcagcaaatcccccttgatgtgggaagttttaatacaatttgcagagtgtctctctggagtagacatcctcctctgcaatcgtgtcttctatatagcctcagggctttgggtaggtaatcctctccaaggagagtcctggagagggctgtctaccccccttgcaccatcctctaacattattctatagctcagctccttgtttctgtttcctgccttgtttttgtctgagtctgcaattatgatgtaagcaccatgaaggaaggtatgttgccagtgtttgcatcagcatatcccccgtgtgtagcagcgcaagggatatagtgagccctcaatgtctatttgtagaaaaaagaatgaacgtatcaacgaaatctgatacatattcattgtgtctgttatctccatctctcttgtcctgccttgttatcttgccattttcacaaaaggccccaaggcccatcatttcttgtgtaacttccagagtgttaatttttaaattaaaattaaggctttctacatgagtgtctattatttgagaaaccatgcaagatcgtgtgtgtgtgtgtgtgtgtgtgtgtgtgtgtgtgtgtgtgtgtgttgcactctatattatattgaattctggattttttcttataaataaaattttaaaaatagttctttaaaaataggaataagatgttttaggaggcacagagagcaaaggagaataaaaattgcaggtttggggttgtgcatactaattgccattgagtaaagagagcacactgaggccatttagaagagaattaacgtgttttgtttttgtttttgtttttgtttttgtttttgtttttgttttgagacggagtctcgctctgtcacccaggctgaagtgcagtggtatgatctcggctcactgcaacctccacctcccgggttcaagtgattcttgtgcctcggcctcccaagcagctgggattagaggcgcccacaaccacgccaggctatttgttttttttttttgtatttttagtagagacggggtttcaccatgttggccaggctggtctcgaactcctagcctcaagtcatccacccgactcagcctcccaaagtgttgggattataggtgtgatccactgcacctgaccttatttttattcatttaaaaatattaaatgttactgcatagggagtaatgggcttaacaatgaggtgaccaaaactcctatgtaccatgcagagcaatgtatcaaatgtttttaactataaacttctcaaaaacataaacctaattgttctgcagctgcaggttatatctgccttgtttgagcaaaatttggtggtgaaaatgccttgcttccatttttccttcaataactgatatggtttggctgtgtccccacccaaatctcatcttgaattctactcccataattcctacttgttgtgggagggatccagtgggaggtcatttgaatcatgggggcggtttcccccatactgttctcatggtactgaataagtctcacgagatctgttggttttatcaggggtttctgctcttgcgtctccacattttctcttctgcttccatgtaagaagtgcctttcacctcccaccatgattctgaggcctccccagtcatgtggaactataagttcaattaaactactttttcttcccagtctcaagtatgtctttatcacagcatgaaaacggactaatacaataacctatataattttgaaaagtacttgtctaatagactttcacaatagaaactatatccttatcaactttgaaaagtcattgcttaatgcctttggataactgaattttctaagattattttaattttgaaagttaaattttatcccagtgttgacgatttttgtatgctacttttaaaatattttgtcagtgatttatatctatggtgcaatcttgtaaaaaattaacaatgcaaatgtggctagaccatttaaaaatcaatatgttataattcagcccatttaatcactttagttaaacatcttaggaacaactcagttccatttgagagaagacacagttttctagatgtgtgttgtggcatcatattgctttacaatatcttacataaggtgaattcaaatcatatcattgaatctgttttaaattctgtcatagcttaagattagtgactaaatattggcaggtttatggaagtaggatgtaaacaagacaaaaacaagggtggaacaagtaattttagtatattattcacttgcacagagaaaagtcattcacaccttcttcagctttgtgaagaaaatagactaaaatcctgttgatatagcaactatgttttccgtttcttgtataaaaataaagaaaacttcctattaggaattagccagacattttaattttctctcttctttctctattttcccttacagtctctttgaaggcaggcaaaatttctataaagttttaagaatgttttaagatttttttattgtgaaatattcatagactcacaaggagttgcaaaaacagtacagagatttcctgtgtatacataacccaactttccccagttacatattaaccaaatacagtatattaccaaacccaataaactgacattggcacagtgcaatcaactagactgtagaccttacttggatttcacctgtttttgcacatgctcttttactgtgagtcattatctgttattctatgacattaaccatgtctatagatttatatagttactaccactatcaagataaagaagtgtttcatcaccacaaagtaacttaaaggattatttttataaagtaatgacaaatgtgtcaaaagccattcctgtgttatatagcaagtatgttttgagttattaaaactcactgatcatgtctttcagtgtcataactttgggtttccctccctaactataataatcctgatgaattacagttgatgaatatgagaatatccaactcttcctgactctataaatatattgactgagattgtaatatttatggtgtcttaaggggcgcttgttttattatgatgatgtgaacatgttgagaatagtaagaacagcccagtttagcaaacaggatatgagtcttctatatccagctcaatcgttgccccaacaggggacatctgcctttgctacttaattttccattctggaaaatgtgaagtgtatgagaatgaataatcgtctccgattttccagcacataataatctgaggagagcaggtacagcaatttaggagctgttttcttttggtttccaaaaaaagttccgtccagtggtctaagttagtcgtttactaagtgatagagcaattggctatgctttttgaacggactgataattatgtggatgcagcaaataggatatagacaatgcatctactccattacagtaaaaaagactctgatagcagttaatccacataccaggcacttagcttaggcacagttggaggaaatggaatggtaatagactgtagtatggcatgacaggagctgtagcttgagattcagaattccaactctgcctctcaatatttgagtcctcatggccaagatatgtaaagtgctctgtgcaggtcttggcaaccatccaccacacacttagtatgcaatatctatctttattagtcaaggatctggaaagctagttgatgagacaaatgatagaaacaagagttcattagatgaaataaagtaataaatgatgcaagaatttaaaaaagatttagagaaggaaagggaacagaactcacatgcaagtagagcaactgtgtatcagataatgtgctagctgagttagaaaccatgtctcatattaccctgaaaataattctgcaaagctgtaggtgttatttttttcatttgacaggtgaattcatgaaggcttgaatatagggttaagtgagttgtttcaatgtagttattgattcaaatcaagatctgaatgactctaaatatggtgctatagagatttgaagtaggataaataggatttgaaaaaaagaaaaaatatatagggaaaggaattggtacactgtagcagtgtcataaatgaagcttcagttgtgtgattccagatgatgtatgtgaggcctaatcaaacagctttgtggaatcaaaatttctgctcttgtctccaactggggacgagttggctcgggattaaggtgggcgaccttgggaagactagagtctaagcaggactttagtccctcataagaattatatgaggatgtatatttgcatacaaattcctgggcccaccgagatctgccaaattggaatgtgtggtgatatcacccagggaaacatagagagctgttataattagtcatgaaatatttagtactgaaattatagattatgttaaataatcacttataggggacatagcagggttggcaggttaaccatacagcaaacagggttgtaagtcagggcctagagaattttcaagaggcaggaattctgcagaatgaaggcctggtctcatgcagcaccatggacagctccgaggcactcttgtttctccaaaaacctgaaatcaaaaactttgcttttcatcatgcaacatacccatgtaacaatcctgcataggtactccctagtccaaaattaaagttgaaaaaaaaaactatactttcatttgaatacagttctcttcggctttaccagctctactctggaaggaatatcttttactcaatgaaaggccatcccctgttaatgcctggccaggttctccttatcagtcattcactatctttgtgtgtgagtgactaaacatataatgctatgtttagtggatggagtaagattacctttgcagaggttgtactggcttacccctttggttcttgtagttttcttctattagagttttttccatccctaggtttctatactgttcaaatgggtttaagattcttgaaggtattcctctgaccttgtaatttatgcttgtctcctagcacaacttttttttgtaaaggaggcaccaactatgtggtttgctggcgatggcatacacaaatcaggtgggaggaattaatgagagcagcaattccaatatctggttcttcaagattaacttgtatagtttaattcagcattctaaataagcctcatagatttaaaaatctagaataaacccacatttttaaaaaaagttttatgttatctgtgctgataatgcacgctgtacataataaaatattattttcttttttttaaatttattattatactttaagttttagggcacatgtgcacaatgtgcaggttagttacatatgtatacatgtgccatgctggtgcgctgcacccactaactcgtcatctagcttaaggtaaatctcccaatgctatccctcccccttccccccaccccacagcagtccccagagtgtgatattccccttcctgtgtccatgtgttctcattgttcaattcccaatattattttctaagtggcagtggaagaaacatggaaagttctacttcatccatcggtggattagaatttgtataccatgagatgattaattttcaaaaccagtttgaatctcacaaaataatgaccctgttttttgaaggacaaggcagaacaaggaactaggctgtgccacgttcaagtcacaatctctaacatttgttttgttttgttttgttttgttttgttttgttttgttttgtttaatctctgttgcttgttcactttctcttgtaatctgcattgatttgctacctggctatttgtagattgacttcggctgccaggaatggaatgtttttcataaaggaacatatgccttaatgaaagtaccataagaagggagtagagtgtgaccaattgcctaggtaataagtagtgacaacaatgatattattctagtataaatggaatcagtttttctttgcccagggggcatgataaagaaggcctggctggtatatactaggtgggacacaccaacagtgcctagaatgtcaatggatcaaacctgagggaaccagaagttgaaaagacatatcccaaaagaaagcatttgatgtttaagggttggcttacttagaacacaatgaaaaatattactaaaattaaaactatgattttagctatttttaaatatgacaaattaaatagcagaacattttaataaaacattacttaaggtccacaattttctgtaagtctaatacatgggtcattaaaataaaaaattccccatgatttatggaatcagatttttttaatacaacgaattctaaatggttttataatgccaattccaattaatatcctaattataacatgtcatccagaagggttaatgactaaattttattaatatttgttttctatttattttgatttgtgcagtttatgtgtatagtaacgatagctgcaaattagataccattagcattaaataaggtatatattttaatagaaaattaaagttaagtatttgagctagcctaaaatattcaacaacttaaatttgttttttgtggatcacatttttttgagacaaagtcttgctctgctgcccaggcttgagtgcagtggtgcattcatggctcactgcctcaaccatccaggctcaggtgatcctcccacctcagcctcccgggtagctgaggctactggcgcacgccaccatgcccagctaattttttgtatttttttttagagatggtgtttcaccatctggtctcaaactcctgagctcaagcaatctgcccaccttggcctcccaaaatgccgagattacaggcgtgatccagtgcactcacccctgtgaaccaccattaaatagctaataaaagatgcatgtcaataaaaataaacaacttactagaatgattatgtgaaaatcatttattcttccaaagcatgaattttcaaacacaccttttgttactgttttaagaagggaatcatttccatatatttgcatgtaaatcacttttagtctcagagaactttccataaaagtttttttattactgctgtaaccgatagagctagtggactattaatttaaaaagctgtacataaaaacacatctatagctcaaataatctaggataccttttagtttggggaaatgtagatgaaaatgaagtaattacagaatccttgttaattttcagatttagacagtctaggcaatatctttcaggaatgaagagatatgtgttttttggcatcttggtagagtatattcccattgtaattcttttgtgaagtctagaccagatgtggccataaaaatagacccctactacaataatatatttcatagataatccaataaagtcaaatcttattgcagtaggcttagaactctgtttgcacccatggaatttatatcagtttttggcaaatcctttcatctctgaggatactttttcatctcacatataccctattttctgaacattttgccttcaaagtatacctcatttatcaagaatttctctttattcatctgacttatacaagtggcaataacaacgtctggttcccatgaagtaaccagtgaccctttgaaataatatagcgctggaagaaagaaaaggaaagggagactgatcattcagcaactctttaaaaccatgtcaccgttaaacacatagtttattttatcttttttttagaattgtgaaaacctatattagcatcttcacggatgtctcctttgtttacatccccgcttctgtgccttgcctgcagtagaaaaaaaaaggacatgtgtatccctattccccattgtcttctcattctacatgagaatgagaattcttttaatttcttctctatctacatgaacccacttccattatctgtttgttcagttctttaaatgccctgaagctagctctgtgactgggcagttgaaagttctggacttagcatcaggttaatttgaaaaatacttattgagccaccaccatatgtcagccactactgtagatgttttgaatgtgtcagtgaacaaagcagaaaagatgtatgccctctggattcttgggggtctcaaatagtgaaagacagatacgataagtatattgtatagtatgttcaaaagtgataagtgctgtgaaaaaaaagaagaagggtaaaataagagatggctcatgctggagtacattccaattttaaatagggtatcatggtattcttcattgagaaggtgacatttgagcaaagatctcaaagaatgaggcatggggttgaatcatgtagatatcagcagtaaactcattttgggttcagtaaacagtcaatgcagaattcctaagccatcggtttatctgctgtttggggctggttatctgcagtgtggctagagtgaagtaagtgagagaggtttaggagagaatgttagtgaggtgagggtggacctttgaagccattgtaaggacgtttttctctttctaagtgtgagaagatgatgctgactgagaccagggtgataagaaatagtcatattctgaacgtgtttggaagtggggccaacaaggatttctggatgaattggataaggggcatgggagaaaaatggagtcatgaatggctccaacgtttttgctctgattaactggaagggataaagttgccctaaactgaaataataaagactatagatagaatggggcgattagggaggcattaaatttggatatctgttagacatatcaccagatatattgaataggcaattgaataaatacctttagagttcagcaaaaaaggtccaggttggacgtttaaatttcggaggtgtttgtataagataacatttaaagctgtgatatcagattgtatcactaaggaagaatatagatagaaatgacaacgtgactaaggactctaacattaagaggtggattgacaaaggagaaaacagcacaggataatgaaaaggaatgatcagccaggcatggtggctcacacctgtaatcctagtactttgggaggccgaggcgggcagatcacgaggtcaggagttggagaccagcctggccaacatggtgaaaccccatctctactaaaaatacaaaattagctgggtgtggtggcacacacctgtagtcccagctgctctggaggctgaggcagaagaattgcttgtacctaggagacagaggttgcagtgagccaaaagattgtgccactgcgctccaacctgggcgatggagcgagacttcatctaaaaaaaaaaaaaaaaaaaaaaaaaaaaaaaagaaaagaaaaggagtgatcaatgagatgggaagaaaaacaaaagtgtgtggtgtcctcaaaaactgacgttctattttcaaaacctacattttgggtctccttttactatatcctgactttctagctatataaccaaaaggagaaagcagtaatttttttagatataacatgttaataactctaagggtattcaatgaatctgaataattcagtggtataatgtgaaaaaatatagtattcataggaaaaggaacagaagttagctcaggaaatgacttgaatgaacaccgaagccaaatctccagcgcaggtccacgtattatttgtctcagtggttgaattagcagcaagattccttagtaggatgaaaaaagatgttgtgagcatctgtatctacatgactgaattaaattcctccaacaatgaaatgtagttaacgtagtatctcgaaaagaaccctaagtggaattcagggaacctaaattccaaccatggttttgctgctgactgattgcattcacttcaaatctatcattaacctccttgtgcctcattatcctcatttcaccaaataagaaaaatgaaatattcctccttccctacctcactaggatgttgtggatttaaatgtgtgagaagtgcttgagatgcataaaatttgatggagtgttttattcatgaattcaaggcatctgaagtaatttgaccatgatggacagttgcttccttgcacattttttagagtgacatttccgttactgacccacccatttatgcaacatgttgcctaatctaaatttaggtcaaaacaaattgaccttataggtaagcattatatctattaatattgtatttttgtattattttataatattcatcattcacctattttctcatgcaatatatgttactgaacacatatagattaaaaagccttcatccctaaataacaatgatgggaccttccatttttatatccctctggcatttaaaatgtgcttttatagccatcatctccattgatctctcagtcccttgaggttgatatgacagatatgctttttccattttaaaattacggaactgacagtctcagatgactttaccctccaactactgtgtgaagaagcagggtctggcactgaggtcttctgacatccagtgtagagcactatacttcacaatatggccattggcttactttattacaagcactaaatattttccactgaatacgtaatacctagaggagaatgtcgtgtaaaacagcagcagtagaacagaggattaaatgacccattttcttgaagttatcttagttttaaagggttttttcttcatcactaatgaccatccctgactaagaaattattctcataatacatgataatatctgcgttttccaatgcgacaagaatgttaggatgtctatacatgatcttgacaatccctagctccatcacaatgtgtccaaattcattttatttggctagacaggcatgtagtcttactttcaatggttggctctgctggatgctatgtgatctagaacctgtcacttaccccttctaaacttcaggaattttttatccttaagataacaagaaaactcgtacctgtttcaaagagctgtttgttcaatcacctatccattgattatcttctatatgccaaatgtttttctaggtgctgaattacaggaatgaatcagaagcaaaaagttcttactctcaaggatcttatatgctaatgaaatagatgttaaaaaataacaatttttgtttcattttattttattttattttgttgagacagagtctcactctgtcacccaggctggagtgcagtggcacagtctctgctcactgcaacctctgcctcccgggttcaaatgattctcctgcctcagcctaccgagtagctgggattacaggcatgcgccaccatgcctggctaatttttgtatttttagtagagatggggtttcaccatattggccaggctgttctcgagctcctgacctcaggtgatctgccctccacggcctcccaaagtgctgggattacgggcatgagccagcgcacctggcattaaaaagtaataacaatttttaaatatcaatatgtcttatacagaaaagtgagcagtgtggtagagtgtaactggaatgtgagttgagacataacaccagacagagaagccagagaaggacttttgtttgaggaaatgacatttgaaaaggaacctgaatagtgacagaggcagatacctaaagaatatgttccagacaaaggaaacaaaaagcgtgcaattgcatagtcaacttagcctacttgaggaaaagtgtgagtggattttggtgatggagaggtaagtgccaggagatgaagggagagatctggcatgcatcagatgatgtgcagtcttccgggacgttgtaaagagttgggctttttttgtttataaattaaatgttaagccattggggtttttaaccagaggagttatgtgatatgatctatagttaaattatgtttgttcttggatggagtgtgcattatgggaatttatacagaaacaagatttcacatatatatatatataaaactcagtgtcaatagaaaataataaaaacaaattttatccattgataattctggcattgatagtagtgggtatggtggtaataattgtgtgtaacactcaaactttctgaaaacctacacttgatctgtaaatccaaaagtatatgtagcaaaagccataatctgctcttatttctgcaccacttgcaccagtgtggagtgataaggcaaattattcaggcacctgtgtaagccttcagtgtcctcacccccttgttataactctccactaatattacattggtaaagacgtccctgacctatatgtcactgagacctcaaagaaaagagcaaagctaaagcgtaagggggaaaaaagccagcttaaaaagacttaaaggtttctgggaccaaaaaaaaaaaaaaaaagtctttgaaaaatgagaaaggaaggatagaagaaaagattctcctttggtcaatctggccaacctttggaaataaaaagtattgtgttgcagctaataactatttgtcactgcaggcacttgctgatgtctgccctttaaaatgacccaaactcgttggcctcgaaatcagaagccaaggaaaaaatcttggacataatgttttctgtagaattaccaattttctctctctctctctctctctctctctccctccctctctctctctctctccatatctatatatatatagatgtatatatattttttctgtaggaactaccaattcctatctatagggactgattgagaagtcccttatagcagtttttctttggcttttaggatgcaatgattattggtgagaataactctttcatttcacatttgtcattggcttatttgaatgtaatcctgattcaatcgttatgatctcctttaagtaggaagagaagctggtattacattgtaggattttaattttgtactcatgaaacttttgaaaaacattactcatactcttctgactgtcaaattggcctctaagaggtccacatctcaagaggtatcaagcattggtaactattttttggtgttgttttctcatcataaaatgtacttttattaggtgactttggaaattttattgaatcaatgcatgacactgcctcattctagtaatctgatgaagcaaagctgaaaaacaaaatttgaggattgtcagtatatatacttttatttgcagtcaagagttatgctgcaaaaatggtttattgaagtaacaaaattttagctgatatattaatctgaaagatacagtatacatttttagtatggaaaagatgaggaaaaggaggttctctttcctctaggtatctagagcaaactgtaactgtccttggtatttaatttttggctaaggtactgagattagaggtggggccttagatatgattaattgtcagactgataagctagatatttcattgagtttctgttgtgctctttctttcagatcctctgttcgatgctttgttataaagatttgggcatttcaaaatcttctccatatctggtgtctttccaaacagcaggtcatagactttacacaaagaggaacgacacaggttataagtagaagtgttttaaaccctgagttcctatttcagttttgctttcttaaacatattttccttatgtgataaatgcgagtgttgaatggtgataaataccacccataggctttaaagcctaaatgttgaatttgacactgagagtttaaaggcatcatgaaaatttctccagaactaatgttcaagcaatttaggttttacaggcaactcaatagttttgaatgatgtagttattttgaaaaagtcaccataaaacgctatgtttagggaattggtactttgcatttatcagaagattgtaaatgtcaatcgattggcttgctatttggaatataattttttaaattatagttcaaatcattaggatttaattcatgattttgtactacaaactaaatctatgaaaaatatcagatatttattttaaattagaggcatgtaaaggaaaatataaattttgaaatgccattttactggatttttctcttcagcccaccctaggcatttgttacataaaatatttctgaggaagtcttccactgattttgtaaacaaacatgttttattgaacagttctttgttgactagattaacattgaccattgtatgcaatgcattctcaaaatcttagaagctggttttctttttaatcatataattttacttgttttacagtgaaattaatgcatgtaaaaagtatacctatatagaaagttaaaagaatattgctaactagttactatacttccaaattgcctattttctgtgtcttgcattggacagtagtgattacctctaaaagaaaatggatggtctttgtttcattgaagggatggataatggacataactggcattcttgagcaatgcaattgcaaatacatgtctttgcatttatggtccaatcatcttcttactatgatagcatataattgaaggttcaaataaatgcctcgtcccttcctgtggcatattaaagagaaagaaaaattagaaatactttcaaagctacctcacatactaatggtagagttgtttgagtatttaggtgatttaacaaagctgatgtattttattatgcttgatcattgaggaaaatttatttatcggaatgcttttgagagcatatatattgtcagagataaacacagctggatattaaagaggtaaaaacagattttattcaatacctcgtgaaattaggggagagctgagatccattctaatttgtgcagaggcgacttggttgttttaaggcaagaaggagggagaaggagtgggggttcattcgagttagagaagtaaaaaagtacaaagggctggacagtgtaaatgtgattaggccagctgtgttagctggaagttattgaagttaggattctatcttcccacagagaacaggagacagaggacttatccttcttgatgatgtcatttgaaaagaatggctttcaggtccttgagtgagagacacttctgatttccaagagctacatgttcacaattgtaagcccttttgagtaaatgttctaagaaacggaggtaagagtcctatcaacagatgtgtgttggctagaacaaacattaaattttcctggcagcactgagctttctcaagcaggcacttaagggaaggctagggtcatcctagggacatggccttctggggctagaaaccatactagagtttagtcaagtcttagtgcaagggtttggacagagttgttaagtgctgagagttctgtatttctcactgtcacaaaggaagatcagaagctcctgatacttttttcatcagtacaattgaatatataaatcctatacacaaaaataaactaagcttatacaagcatattggtcaaggaatgttgctggccttattaattagatagcccagttaaaagaagaattttttaatataattaatgttaaagtaggatgatagtatataaaacgtgtctactgtcctgaatacaaactaaactgtttggtttagcatttacctcaagatctcttaatatcccccaaagggtccctaaaaccacaacttatctttgtgctcatgaagtagagaagagacagttaatagacatttctagctgatagactgttgtagagcagagaacgctctgtgtttttgaaaattaaacatatgaatttgcccctcttcccctattaaggaagaagagtttcttaattgtgcgaacacatcaagtgaactattcaattagatttttgtgacccagggtataaacatctggttaaggttacatatttcaaaggaacaaaacactagaaactcttggttttaaatctcatggctggaggataatttgcagcagagatttatctggcaagcatacagaattgctgagactgttctaaagatgtaagtgtgggtgtttgtgtcgtgaaaatagctgtttacatctattaagtggataccgatggttgaaagtgccgtctatgtcaagtttttaccaaatcaacttttgcctcactgtgtcagaccattttacctaatcaacttggactgctaatgtcctttcccctggcaccactatctgtctcttttgcaaagcacagaaacggcatgcatgattgtagtttataaaacacatgtaccaatgtggtctacagcttctgttgagttcgagagggtcagtttctgtaatctcttctggcacagagtcaagaacagcttcactttcctcctgctacctctctacccgtaagtgtgaacccatcactttgctaacactcaggaaggggattacacaaaatagagcaggagccctctgacctgaatatgcatctgagccctagccatagagcttctgattcagtagatctgggatggggcctaaatatttgcatttttaagtgtataagtgatgctgatgctgctggttccaggaccacattttaagaaatatcgataaaggtggagaattaaactgcagctcagaagacctgagttcttgccccagcttgacttttacaatctagcaaatggataaaactcgcaggacttcagttctcttcatctacacagtgagtggttagattggctttgtaatttaaaattaaacagggtttgattctgattcactacacaaggttccaaagaaggaatgatatctcctttcatttcttcactttgtcttctgtccctaggtaatcttatctatgttcctgatttaacctaactaatgtttctgcaaagcttctaatatttacatctccagccctgaaactctcatttgaatgctagtcttatatacatacccccctgcctaattgacatctccacttaaatgtatcagaggcaactcagactcaacaaggaccaaactgaatgttcgaccttgtccttcaaacccgatacacatccaggttcctccatcccagtgaatgacactatccagttaagcaagccaaaagtctggattttttttcctcactcttcctcactgtccgtcaactaccattattaaatctgtcacctggtcctactgatttaaccttctcaatatctctacagtttttctttatgcccattagtatcctagtgcaagctaccatcgtctctcattggaattaacacagtaacccccctacccaccagactgttctgcctacagatagtgtgatatttaataaatataaatctagccttggctagatttctccttcaaaaggttcacattaattttagccttaaaatggtgtgcaaagctttgcatagtctgtcctttgctatgttggcagtattttttactatccctctcatctgctcattctctgtactccaactacactaactttgtttttttttttttttagatttctctaactacagtgctgtaatctctttttcctttgcacgtactattccgtttgtcagggaatctgctcactgtctccacccactccacacactcacgttttcctgcccgtcttaccggtcttgatcggttgtcacttgctcaggaaggtttccctggtcaccccctccacaaattgaattaagtcctcttgctgcatgctgtcctagtgctctttattttcctctcctcatccttaattcagtttgtaattacatgttatttgtgtgaggatttgattattatctgtgtcacccactagatattgggcattctttacttactcaccactgaattcatagaaccacagtaattgtacacaacaaatattcaagagaaatttattgaattgatgaatgaaaagttgtaccttaacatgttcctgacatgtatccaaaaaagagctcccctttggggtctattaggactttggacctaggtaaacgtaaccctagtttcgctcaggtttaaacagtagaaagtaattgggtctcttttgcatgtggctttcctaagggctaaccctgtcttcggaatgagtcaatacagcagagctgttgaaagcagactctagcttcggacaacgttggtccgaatcatggttccgtcatttcttagctgtgtgatttagaataaattaatgttttaaagctttgatttcctcttccttaatctggagatgctaataaagccaacttcgtagaggtattgcgatgagtaaataagcataatttgctgtaaacaccttgcagattgcctgttgtatgctaactaatcaataaattgaagctcttaacatcattatattagatatttccagcattgagtatactatcaggcatgtggtagaagctcaatataaagttttgttaaattgaatagattccatatatggtatttctacagcattatgctccttatttaagtgtctctaagtattttttaagtatcacctcacaaaagacagatgtttaattcattacacatgtgaattgttttagatagaaaataaaataaaaaattcaaacattgaaatcaatagtgtaccttaccttaggattacaccataaaatttctaccaatcgagaataaagtgtacagtctatttcctttctaatacttttaacgcaacaaatgtttattgaacacttactacttctaatctatgacagacataaagatgaataaagcatgccacaatgtttaaaggagctcactatatcataagaaagcggattcacacagacaactctataagataaagtggtaaatttaggctggcctgtgaaacaaaggattataggtatagttaagaggtggaatttattttacttcgaggatttcagttacctttatattctttgtctaacctttcatgtttctctttcttcagaaacagagcacctttttcctgacacattcatttccccctatggagtagagcagttgttttcaaagtgtgggtcccagatcagcatcacggggatggttagaaatgcccattcttgagcctcacaacagacctactgaaacagaaattcttggagagtggagcccgcagatctgtgatcaagccctgtaggcaattctaacgcacactcaagttaaagaaccacgggaagaaaggtccatcctgtaacaagacagatttttttcattagcatcaattttgatcatttatatatatatatatatatatatatatatatatatatatatgcatgctcacaaaaccattcaccttactaggttttagtattccccttcctgtattcatgtggtatgtatgtatacaagatgaacacacatttacctgagacaaggtaagactacacatgtctcatttggggaccagaggctgtaatcttactcaaggtcaaagcgtcttcactgctttctttcactgcttttcaaaagtaaaatttccatgtaggtgtcatttgttttctttttgtgttttagaaaaccgattaaggggtgaagtctggctaaacttagtgtcaggacatttacttagataaaattattttaatttatcttgtaatgttcaatgtgagaagaaaagtccttatgagtagtgtattccttaaataacaacaatttaaaaactaccactgaagtctgtcagagtagttttgcctcatttgtctagataagagaaaaaaggttcacattagggattgcaatttgtctgccaaagtgcagtttatttattcagaaacatttagagaggaatgtgtcagttctgttgcaggcactgtgctgtgacggggagctcaagatgatctcaaaaaatttcacagatggggtgggcagggggcacagagagatgtatttagtggttcagatactatttagactgtggccagcatttctctaaatgcaatccagataacaccttacagaatcatctgggcagcttgataaaagctgtagactcctacccttcatcccaaacctattgaatcagtgtctgtgtgtgaagacctagattgtgactggtaattataccaaagtcttagaagcaactctaggccagtaatactcacatcagaatcagctggagggtttgctataccacagattgctaggttagccttcagagttgctggtccagtaactttggtgcaggtccagattttgcatttccagcaagttaccaggtgatactgatgctgctggccttgatcgtgctttgaaaaccactgctttagctacgctataggaaaaaccatataaggcttttatactggccaatgacttcacaggcctgaattttagaaagcccccttctgcagcttggcctatagattcgaaggaaacagaactaacacaagaaagctagttaggagctagttaaaaatcatcctgacttgccaaggaaaggtgctgaagacctgggtcacagagcaaatgcaaaacactaggactttgtccctagttcaccattaaatcaacttattttctcttaccccctcatattcacgtttactccttactttgtagtggttggacaaaaatcaaataaatctgagaattctaaaatgcacacccttgtttattttctaactcaaatatgccactgttgtctgtgctctgtcaagatttcaacacatctttttctcctgtttgcttttccttttggcatatagtgagtgtgtgtatacacacacacacacacacattttttttgactccttccaatgcccttctgctctccgcagatacacttctgcattctgaataaaaccgaatacatatatatatatatatatatatatatatatatatatatatatatgcacacatattttgaaaaccttatttgaaaagaaagctttcggaggaaacgttatttagccacttaatcgagtcttttactgagggactttttgtcgtcccctaacttcctgtcagcagtccacaggcagcaggaataatgtgggagaagatcaacaggcttatttcaggaggtcaggggccagtgccaccacctgcaggtggagacatcagaagcaggaagcagcccaccagctgcagggagaactccccacagagcctaaccaagatgaagggacttgtaaatttcaaccctcccttttggcttttgtgctaaaaatgtgaatattgaggtctgccctgattaagaactagatacattcctctttgtgactgccacacttccttagcgtattcattttttgtctttcgatctcaagttattattttcaaatgcattgcacgtatctaccatggataccattgcaattggaaggagcaaacgttttgtatgtttacttgacaaagagaagtgactgcccaagccacacagagttctgcacaaatcagtaacttctaacgaacgtttgcacttccgggcttgttctctacctatttcagtcgatgcatttgtattatttacttcaaactccaatactaataatgcctcaatcaggttgcaattgggatttgagcagccagaatttcagaaatttggtttggtccatatctgtgacaggtcagtaaatcagagaagcaagggtttggttgctattataatacattgcttacctatcaatttagttatcagccaaggtggttgttatcatccaaagtggctcattaaccaccttggagactcagtatacaattgcaagtaaccctggaagttgtaaataatcccaactgaatttgtatgagtttggtaaggttaagtggaaaccagctgcttagggccttgattataaatgaagttaggagtggaagaagtaacaaaaccccaggcaaattcattaaacattttttcccttcaactttatgctcacgaatgtgttgagactcttctgaatccataaaacacctttcagcatcatctgggcagcttgataaaggctgtagactgcctgcccttcatcccaaacctactgaatcagtgtctgtgtgtgaagacctagattctgactggtagttataccaaagtcttagaagcaactctaggccagtagtactcacgtcagaatcagctggagggtttgctataccacagattgctaggctagccttcaaagttgctggtccagtaactttggtgcaggtccagaatttgcatttctagcaagttaccaggtgatgctgatgctgctggccttgatcatgctgtgaaaaccactgctttagctaggctataagaaaccatataacatggacaaggcaaatgaaaaggttggaattcttctgaatcccaacacatttgtgagcataaagtcgaagggaaaatgattcttctgaatccagacacatttgtttaaggataaactgttttttccttctgaaaatttaatgtctgattctcgttcattcattcatcaaaagttatcaactatcaactataggtaggaactgtgcaatatgctggtgataaagagatgaaagacacagcccctcccttcaaccagctcctagttgaggtggcaagtcagctgtataatcaagtaattgcaagactgtgcactgaaaagggtgaccacagggtgtgatggccacccagggctgtggaatcagtcccaaaatgaagaatgaaagcagggaagggtaattcagaaagaagaaacagttcgcataaagacccatagataaacatcaatcagatgtggttaagacaaaagtaagtttctggaggctgaggaccttctcagctatatgtttgcagtgcttggtatagggctttatgcatctacatggaagacagaaagggccacatcacagtggacaaggcaaatgagaaggaggcagtatcagaagatgagggtacaccggagatcctagttatatatgggcattgtgttcatctcaggagttactgagtaatgggaccttgactcaaatgaatctcaagtctgtttttgcctaatcttggttttaggactaggattagcatacaaccgcactaggagcctagttatacgaaaggctgcattgcggacctgatacagttcaatatacatactgtcaccttgcaaatagggttacgttagttctcaagactgccaatcctctgtgctctaatccttttggcttttttttttttttttttaactgtctcactctgtcatccaggtgaagtgccctgggatgatctaagctcactgaaacctccgcgtcccaggttcaggtgattctcatgccacagcctcccaagtagctgggattacaggtgctctggcgccaccaggccctgctaagttttgcatttttagtagagacagggtttcaccatgttgcccagactgatctcaaacgcctgacctcaagtgatctgcccgctttcctttggcttttaacactatagagcaagggtccccagccctggggccacagaccagtacaggtcagtgacctgttaggaaccggggccccacatcaggaggtgagctgcagggccgccagcattaccacttgagctccaccttctatcagctcagcagcggtattagattctcataggatcacgaaccctattgtgaactgtccacacgagggatctaggttgtgtgctccttatgagaatctaatgcctgaagatctgaggtgcaacagttttatccccaaaccatcgcctcccacgcacctctccccacaaccccacccgcccctgatccatggaaaaattgtcttcctctaaaccagtctctggtgccgaaaaggttggggattgctgctatagggcgatggttttcacatttgatcctgcatcaaaatttccaggtgactcattaaaatactgattgctgtgccccactcgtaggagttctgataaggtagctgtggggtgagacctgagaatttacttttctaataagttcccaggtcatgctgatattgctttgataaccaaagcaatatcagctttggttatcaatatataaccaaagccacatagagggggagaagttccttgggtttagcccagtgtttactgcgaccaccaaaattgctggagcttaaccatggctcagagagttatgttctgttcactctgtaggctgctattccctgtcaccttttgaactatgatggaggggaagagctgccagctcaggagatttcacttttttctctgcataattgaaaatccagaaacacagggttttgggaaagctatagaacagatcatcagtgatcagtgtttaataaagtaaagcaataaactttactgtgtaaaataggatactttattatataaattttgtccccttcccccacctcacaggccaataaaataatatacttcttgtccctgggtgtaatgttattggaaacctttgaatgtaggagaggcatgggcttgtaagttgcagaaaactgctagcctaggattgagaatttcatggataatccaaaaatagatgattttacagttataagccttacgtgaacttgaggtaagaaaacacaatgcctttatagtcttctcagttgctccacatgccctctgagattctgttctgcccagcctctctggttgtcacatctctgggcattaacagaaagttcacatactctttgtctctgatgataatccttctaggtccatatagaagatccctatccaaaccatcccccaaacaaacctattggttaaatattttctccaccgaaggcactttcttagattctaagtgccctgtaggcaggcttcctctctgatttgggagagtacaaattgcgacaaggttaaatcatagcctgggaatttgacctaaaattcactcttctcccatatgcattcatgaaccttctgctggtttttaaaagaagctacttaatgtcagctcgaagaggttggaaggggttaaaaacatgagcatggcagtaagaagatttatgaaggatctgagaagattatgacttgatcagatggtattttgtcagctagccacatttgtgaagacttgaaaactagggaggcttgtccttctaagagggggcactgctgggacctggattctgtggaaccgtattagtagaataaacaataacctttgcttgtatcaaatgaacttctattctcatgtgtcttttgacatatttttattaatcatatcactgggacctccttgctgaaagatatctccgttccccattctgatgactcccaactaggagtgagatcaaatgaagatggcatggaccatttctccatgtgacagctctctgtggttgccttttaacacttctaatgccctttctcttaagaattcccatttgtcgtctggcactggtgctgtgatcaataaaaatgtaatggagtgaggcttagaaacatgaggaaatttactcaagctatccatttattgatgtgtccatttgtgttgtcagggaagaaaaactttttcactcccctcttaggttcattacttggggggctgcaaattaaactgacgacagatagattggcaatagaaaagacaaagtttattcagagaagtatgtgggagctcacagaaaacatagctcaatgaagttagaatttggggcttatgtactattttaacaagggttttgaaaagaagagtgttagaatttcaagccacaaagttggtgggaaatatgaaagaaactaatgaaaggtaatgtttgttttagtaaagtctgtttatgtaattttcttttcccagcgacaacttctcatctctggtgacaggagtcactctttacccctggtgcaagaaactttccttaaaggaggatttaaaacagttgaattatttcagaaatctttgcttttaggcagatagggggagtacagaaaaagccccttcccgtatctgttgatcctcaaatggctttagctcaaaacaatttttacatcacgatggcataatgtagatctcttcaatgtgttcatttattccacagatatttgtgaagtacatgatatatgccaggtacttgggatacaagaatacataagtatgtccctagtctcgtagaacttacactctagtagtgagctagagaataaatgatattatttattatatgcatacacatatgatttcagatagtgatccatattggaaataaagctggttaagggaatagaaaatgatattgaaggtggacttgtttagattgggtggattggcatggcttctctagggggcagtatttgagcagatatgagagcagatattctccaatttgggcaaaaacattccaggcagaggaaacaagggcaagggcactgagttcaaaagagacttgacctagccaacaaatagcaaggattccagtgtaagagaaggtggggaaggaaggaggtgcaagtataggcaagggcaagatcacacgggatcttgcaggccgtgataaaagaatttaactctttcataattttgacaggacatcattgaagaatttagaaaaatagagtggagatacctgatctgctttcttcaaagagttcattcatcattgctgagtagaggttagactgaaatggaagcaatagtgaatacagggagatagcacaggaagccacgttactagtccacatcagaggtggttcagactagggtggagtggtggggtcagttagagagctggtatttaggatacattttaaagacaaagctgacaggatttgctgtgatgaattagatgtaaagtatgagaataattgagaattatttctaagttctttgctggggaaaagtggaggaggaaaaagttagggtacaaggtgtgatgaaatcaagagtctctcttattatcagagtctcattagatatccaagtggaaatgctggaaagaaagttgggtagatcagtctgaagctgaagacagatactgtgactggaataataacgtaagagttggccggacacagtggctcactcctataatcccagcactttgggaggccaggataggagaattacttgagcccaggagtccaagaccagcctgggtaacacagcgagacctcgcctctacacacacacacgcgcgcaaaaattaatcgggtgtggtggcacatgcctgtagtcccagatactcaggaggccgaggctgaaggatcacttgagcctgggaagtcaaggctgcagtgagccgtgatcacaccgctgcactccagcctgggcaacagagtgagaccctgtctcaaaataaataaataaataatgtggcagtcataggcccttagatggtttttaaagacatgggactggatgaagtcttctaggaggagagtttgggaaaagagcccgagaattgactgcacctttcaaaacaggaggaagaaaaaaaatactcaaaggagacaaaagcaacttctgtgatttatagagaaaaccaggcaagtgggatgaagaaagtccttcatgatagaatcaaaaacagtgtcaaatgttgaaaatacaattagacaaacacaaaagaatagaccattgggttttgcaatatggagctcatacttgaccttgataaaagacattttcactggaagcatgcatcaaaaaactatttgtggtaggttaaaatgtagtaggaggtgaggatatacagacagtggctttcactgtgcagatactgctgctcatgcactaattaaaagacatttgttgagtatctactatgttgtatccattgctaaatagtaacagctgggtttagtcaggtagaacagcatcaaaatcattatagtatcccaagataggtacagtaaaatctgtgaaggaatcagagtagtctcttctccaacagagcgtaagacccagcttcacggagaaggtggtagattagctcatctgggaggctgagtagaagcttgtcattatagagggagaacatcagaagtgtggacaacagcttgaataaccttgaaaggacaaaagaggacggtctgccctggaaatattaagaagtctcacatgattagacacaagatattaggggaaaggcataaggtgaattgagtcaatgaggtcaaagagaagctagctggaggaacaggcgatcataaaatgagtaaaagtatatattcaaagattctttttagaagggctacacaggatggataaggggagagagagagttgaggcacagagacaaattggaaaggtgcaatcataaccagagacatgaaaaacccatagaaatctgatgtagattatgtggtccccaaggttgaacaattaagtacgctttcagttgttatgcccatgatattaacatattttataactgcaataagtgctgaagctaaagataaatacaaacaatgtaattcttattctgtgagaaaatgttgtagctggaagttaaacatgtttcttagctaaagaaaaatattgtgtgatctggattacttaatgttataatttagcaacaaaatgttgacattgagccttgcataatcaaaaaagtagtctattcaataaccacattctcagaaaaaaaacaagaaaatattagaaacaatgataaattatcgtagtaatttaattcagtattctattgttttatttggatttaggaaaggcagaaatgttgaaatattaatatatatccctgtaataatataatttgtgtctgagaggtaggaatgagggcatgaggtcaaagtttgataatgaacttcaaagctataactatgatcaggaaattaaaattggacaataaattcctagaatcgtcaggagttgcttgtgaaatcgagaaaggaaaggatatacacaaaaataaagaacagccaatgctctcaaaggagtctaacttttataatagtcttctgtgttagagctgaactcttctggtttagaaggacactctgttgcctggaaatagggcatggaaaaagtcatcagagtcatgtcatctttcattcttcccatgaacgaaatcgaggccctgaaaagtcacctgtgtttgctgtattttattgcaactaagatgtgcatttttaaattgatacataataattgtacatatttgtgggatacatgtgatattttgatgcatgcataccatgtgtaattatcaaataaggatatttctgtatccgtcacctcaaacatttaccattgctttgtgttgggaacatttcacgtattttattatagctattttgaaatacaaaatagattgtcattaactatagtcaccctactggatgcaccttgtttttaatatttctgaaaacagatacgtctcataggtgatggtgtcacagctgtgcattagttattattgcctgtgcaggtgcaaacgtaactattcatattgttgtcaattaattaaatagttacatttatttatatgcgtttattatactaataaacacaatattgagatagttgagctctagttttgactctgctgttaactagctgcgttactttaatttacttaactaatttggctttcaaattcctgataagtaaaattacaacatgagtttctcctgctataatagcctgagaaatcggtgaaacacatgaattcagatgttgatgctatttaatagcgggattccagatatctacttgccattatgggagggagagaggaggtggactggaggctgtgatttccctaggaggttgttaaaattggccaggtgaggaaagctgagacagaccataaatatgaagcatgatacctagccctcagtgttgaaagaaaatcaaatctcatctttgtggtctaaatatcagtatgatacaatcctctgtgtagacatatcctctgccctattgttttctttctaaaagctaaagcccaggtgtgatcacatccctccgttatttacaaatttctgatgatgatgattcttctaatatctacattccttaccattaccatgatgtccaaaacctattataatctattcgtctccaagtgccatgttgtggtcaccctatgcaccctctaaacccaccatatgaccttcccgctgctacttgaatacagttggccctctacctcgttgtgtctttgcattgcctatttaattgcctttccattctctaaatcactctttcgctggaccagcaacatcagcaccatctgggaattcattagaaatatagatcctcaggcctcatctcagacctgcttgatcagaaacattggagagtggagatgagcagcctgtatttttatcagccctctaggtaatttgatgcacactaaagtttgagaaccactggtctagagcattcttctttaactctcttctaaaaattattagaatgaattcgagggacgggatctccttgaaagccaagaacatttctttgtcatctttctgacttcagggcgtagtacactttttggcccataattaaagctcgataaatgcattctatgccaataaatcagctaatcaaatatattattcatgcccttgaggtatctgaaatttgtttgcagaatgtaatatataactatagagtaacaagagaataatttattgccatagataataaaacaatatcctctgtataataaatcctagcctctgctcaatgggcaaaaacgggactggggtttcagattttaaaaagattattggtaattaaatcacctggagaagcacttgctgcagagatgggacttgaagcatcataataaactgttgtttattatgattcggtcagagctgatggaatcacagggattgtgtgaggtatggaaagtggttgacattgaattccaggctgcacagttgggacttgatatgataaccaaaaagaaagaatgtctggggtggtagcaagctctaaatttagacaatctaggcttatcctaaggagaatatagatacagataactgaagtttgattaaagggaacctggtgtatcacaaatagtaaaaagctgtagttagtctatgcagctatcagctagccacataatacttttgggcaaatacattataaaccaaaagaatgacatggcttatctctgtaacaaagtggctcattgttctttattctactgttatccttaagaaaaaaattttagtaaatttgttatgctatactcaacttcaagaagggatagcgcttataaaaaaattgtttaaagaaacaggcctatttctctttgggagaagccacggagaaacgaaaagaatggaacgtgtgtttctgcccagatggcaataaaatgtagggtaaatttctgtcttttaaaactgtattttttccatccctctgtatatacacatatcctaggactgttataaaatgctgcatgcgtatgtgaaaatggaaccttattgggctgtttgatggacctttaaaatatatttgttggtttggggtacatactagctatgcaatataatccgcattatttcttatgtaaacaatggataaactgtttcacagtccagacatttatttggtcactgtttgtagaatgtctattttatttacttctgaatttgtattccagagatctgccttcaatgttggatacttccactgtaatattctaggagatgctcactttctttttcagcatctgacacagtaccatctgcctcctcttttcttgccacaagtaataacaattttataaaggaggatcacattacagaattataggtggtaaactttctaccaccagatttacccaagaacctgaaacacattttttcaaaaggaaatagaatgtccttcttgtgactacatcggaattttgcttgcagcattatgctttttttttccccctagtgtagctagccatgtggaactgaagccattagccagctcctcatcctataaatgctattacctgggaaaagaggcagaaaatatactctcttctccagttagagtctaaaggaagagaacaatatgggtagttgtgtttaccacaaattgatagaactcctttattttaaatgctaaaaccaaataacttgtttatatgacttcaacattgactatcacacactgttgcatgataacagagtgaaaactacctctattggatttaagtggggaatctatgtctcattctcattctttttttactgtggaaactagttgattccaggatcagccttagctccaacttgccacactttgagttttggtttttcacttgcattgtcacaggaaacttctataggataaatcgaggaagattttactctgcaacgtgttgcagaattaaacatttaaagtggcaaaaccttcgtgtgtaggttgtctccccagagaatgtaaaaatgaattgaaggcagcacctaataggtaaacgacagccaatcaaacaagaacaaatgaaatttgactggcaaaatcaaattgaaaatgtataacgctgaatctcagaatataggaggatgcatagaaactaagctgtactattataaaagtcatagccattgaaaaataatgactggttaatttggttttctttacctcatggatgtgaatggttagattttgatgttggtgttatttgacgtgtgtttgtcaagaagttgccttagtcggctcgcatttaggataaaaaaaatattttaagaaatgtttaagagattatgttggagacattagaaacaaaataattatgcagagggcaggactatcaaaatataatagaaaaattacaccgctcttttatgatttcctcctttttggcatttaacacaaaactttatgattacacacaccacgcactccagaaatgcttaaaggaagatgagaggaaaattcaatagaagtagcaggcatttctgtgaggacagcagaatgatcacttcatctctgtattttttttttttcaaatttctgtatctgtacaatgtcttttccagctctaatattctgtgatttggtaatttccgcactcagattttctttaatgaattttgtatgatattacctatttttataccagatattacctggctctaatttctttttcaccctaggaaataaaagtatcgggtgaatttcccattttcttatgttattgatacaggtctctgttggatatccccacgattaactttcctgcagcatgttcgatggtggcttaaagaagaaaccatgtatcagagccccttgtctatatagacttttagataaagagaaatacatatcacagaattattctgggcgcatagagtctctaaatgcaaaaaaaaaattgtattgtagctgttgattcttctcagatagattgagtgtagagagagagcattccaaaaactgagcagaagaaacacagtctgaatcaaataacatgaaattttagctaacaagtaaataacacttttttcagaatatgcaaataatattggtttattatgaaaaatgtataggctgatagatgagcatagagaaaaaattataaatatcttctttaatatcactttccccagcaaaccacttttaacattttgatacattttcatgttcaaacatttcctaatagtcttttttcctgttatataaatatgaattttaaacattcgtatgtttatgaaaaggcaataagatactgctcttttataacaggctttctgaacttcacaacatgcagtgtattctaacatgctccttgtgttcttaactaataaaaaacctcacgttatttaaaaaaccatcttaaacataattatccattaagagaagaggttggggtagagagtttcagactatcaatatcaaagttatattttctgtaagtattttaatttttaagtgtagctataggtatatgattataaaaccaatagcagagaaaagataccacctttgaatatagttttccttggttccatgaaaatggcctcctttctttttgccagtccctcagtatcattaactcatttttctgtaaatgccatcattgtatcacatgtcctcaggaaaaggcacttttctcttttaagctagtgtttgttcttgttctaattttatggcaatttaacgagtaacaatcctgtttctataaatactgtttcctaattaatctattgcattctatccatgagaatttagatgactttctttgtaagagaaatctctgtagcatgagattcttctttgctcttaaatttcattctttcacatttttaaatgacctgatagtattttgttgtatttgtgctgattttttttaaccaatcttaccttgttgaacatgtaagttgtttctaatatttgcaatgatcaaaatgtggatccaacttcactaaagcgttaagaatctaaaacaaaacaaagaacaaaaagttggctgtcatcttgcttggaccaccccgtgagttactattttcttgtttccggtcacagttcatcctaaatcatttcagtacacaaaatgttttttaaagtttgggacagggggtagagaatgtcaattattcctccaaggcagtcatatgagcattgagtatcatgtggaatagttgttacttgtaaagttatggggcatcaaacccagtcaatatgtttctggaattgaaaaagtccctggacattctaatgatactgttgttcactttgcacctactgttaccactactttgatctgtcaacactgcccgtaatggttaattttgtgcatcaacttgactgggctacaaggtgcccagatatttggtcaaacattattctgggtgattctgtgcaagtgttatcagatgagattaacatttaaattggtagactgagtaaagtagattgcccttcctaatgtgagcagacttcatgtaattaattaaaggcctgaatagaagaaaaacactgaccctcccctgagcaaaagggaatcgttctgcccgactgccttcaaactgggacatgggctttttcctgccttcagactttaaccacaatattagctgttcttgtatctcaagtctgctctacttcgattggaactacactatcagctctctcgggtctccagcttgcttgttcaccctgtataccttgggagttgtcagtctccatagttgcctccataattgcatgagccaatttcttaccacatacaaacacacacagagacacacacacacacacacacacacacacacacacatataattatatatgtgtgtgtatacatattctcttattccttttgtttctctaaggaaccctaatatactccttattactctttctactgccttagagatcttcaaggccaagagcgtaatcctccatcctggctctttttcctaatcattaatgatcaactcatagccatttagctcaactaaaaataatttgttcatgaagctttacactcccacatactgaggaacgtggtacctaagatcaaacagtcactgcctcatcaaatgcattcctcttcaaccccatacaaatgtccccagatggaactcacaccataaaaatattagatcccattgacttttctgctttctcaaggatcattgcagagcttgaaaaagatggctcctccctttgcctaagcaggttaacttggtgtaaaagtacatgtaagatttggcacaaaggaaaataaatcagttttgcctgggtcctaagaaacatttccctctgcctcatggtaattgtacctgccagttgattgcattactcaagtggagaccatgaagtgaagtggtagaacaagaagaaatccctataattttattaagtatggtgaaaaatacagatatgtagagaaatgactgggattagatggagcaaaacataattcgagatcctgatacaaattgtacttcctggctcaagggagggagcagaacattccctgctacatgggaataataataaatgcctgataaaaatgcagatatatcatagactacagaagctgaagtggattcttatggtcccctactcagacagcctctccttcagatgaagaaactgaagcacagaaagctcatcctagtgtttcatattgaaaaacccattcaagtctattttaataacctgttaccaaaaatgagggaaataatttaactttaatgtttcactttgcattacccttttcctgactagacttctatccttttcttgagttgagctcattaactactatgaaattatggttatgggtagaggttaattttatacctgtccatcttctggcatcttatttacactaaaaatcatttttaaatggcttcattttaaaaaatattatttcagttgacattttaaaagacacatcatttatgtactacagaatatgcattttatactctcctttattaattttattattttccaggtagaccaatcaaatgaatcagaaattcttggttagatctattagacagcataagtatgtttttcatcattaaattaagatgaaaacacaattttactttaaagtgtttgacgtttccagcctttataaagtcaacacttaatcacatctgaaatttgcaggaaaaaattttgaaagccttcaattattaacattatttcgggagaaaaagccactttgccgcagaactttcacttttctctcgtgaattaagtctgatacaaattattcattatggtgaagtttaaacataatagagtctagctacttccacaaaaatactattcaatgagtttctacattgacatctaactgaccttgtaattaatgttgtacacgatccttttattatatgctggattatcaaatatgacttattagcagtataaagacacaaagttctgaaatgtaatttatagccatgaaaaggaactgagctttgtgtgacagttaaatttgaagagatcaggtgattattatgaagcatgaataataatgcatattaaactcacgtttttgtttaaatcattaatatgattgttttagaagaaagtctacctctatcatatgggcaataaaatgtgtataagagcaaacatttgtgtatgtgaaataactcaaattaaaaccagttttccacattaattcttacagtttttaaaatttaaatcatttaatgtatcacacatagctttattcattttaagctataaatgttacaatttctgtttaagctgttaatataagctttgtaagagcaattctgtataaatatagaattgtcattattcactaatagctaccatttatttagtgcttgttgagtgcaaaagtactgcactgagatctttgcatatgttctcttaatgttacaattcttacctgaggcatttctgtttctgctggaatatggtctctctgaattgaacaagggaggcatttttggttgttatgatgaaaggtggacactgctggcactaacgtgtgttggtaagcgactagactcttcatgatgcgtaaacagtgtttcctcatacccctgcacattcaaatagaggaaaaccttgtttatagttaatttcccctagaatgtaaatccatttaacatataaacacaaagcgtgttttgtgtggatgttttttactggagcagggagacaggagaggaaatgcagttttgatagttgctgaatttttcaagaatgcagcaattatagaacaatttctagaagtttcctaggagctcttttccatagcagaaaactaggacttaatagccttgcgactcatggtacttgagtgttccatacaactcacctatattcaggggacatttgaaaaattctacattaaaggggattcttaacataggcgcaagtgtctggcatcttcaataggtcttctggtgtggccatgaaaacattcacacgtttcaaagtattttaaaataaaataaaacatatattgttgtgttatgaattattttctttcttttttatatgatggttagatcactgtgcagacaagtttatgagatctattcatttcatttcagggtggtaaatgagggtgttactaaatgttggttctaaaaagggagacattgggtattacagaattcagaacagctctaagccctgtgcacatttagcattagaggacacaggcaaatctggcctccagtcctggcagcttcttcactatgtatatgatgttgggtgggttgctttacctctctagtttttacttttatttctaagctagggctattcatagttctttatcatgtggttactgtgaagtagcaaagcacctgacataattagagcagataaaatgctcaacaaatattgcttatcagaaggattatgtattacctcccgaaatacatcaaaaatatattttccaattcaaagaatatgtagtacaaaaatcatgcctaaattaacagagttgcagtagcccaaggagagaagataatcattattgatttcttcttcctttttgctaagcagttctctgtctctgcctcctcagttgttgtccatcccactcccccactcccaagccctgaactctgaggggtttgctgccgtggccggttctgtagtcattgctgtccaatgatgaaaacacaaaatactgcaacagaacactatgcctgtcagcttagctcccttctttctgctaaatgacactcaatcctattcttttgttctaaaggatatcctaaatgaatagccactggggggaaaaaaggttatataagattgtgcactgtgtgaaactgatgcaaccagatcaatgatgtgaatttctcttaactatttactgggatctagaaacaggtctctcaacttagcagtgtttacgaatataataggccttccttatacatacatctgaagccaatctgagtcaggaagagtcgtggtctgataaatattttgaaaacttgcatttgttctattaaagcaaactgtttattaatagtgtgccttattttttaaagcaaaacatttataaacagtagtcattacaggcacttcagtgtacggagtgatcaattgttagacctttaggaatcgattgtttcgtggagcttcggcttataattgaaatgtcatcagaaggagtgtaagacatagcttcaggagaggccatttatgcgcttttgttttcagctaagttatagagtcatcatgtgaagaaagattcttctcttagtaaaaatcctttaatggttggaataacacttgatatttaatatttctttctactttatatccacatttattcaagtgctaacgcgtgtggggcagcaatgaagcactttattccaacattatagttctcatatctgcgtatgattatttttcatttatcgttagcatatatataatgatgacttttaaagtacactgtattatattcactggaataatgattagctattaataatttgaacactatccaggaaattactgaacatgtcctacaagataaacctcgtatgatattgtctccaaataacagtgctaaccaagaagagtgctaccaagttcaaaagtaatcacagggagtaacctaaatgcagctccgttgggttaaaaatagtttctctaaattatatgttccctaagtttgagatcgatttctacaaggggataaaatgtttttataaattctcagtgataagtcatgtgattaagaacccccaactttttttccaaagacatttgcatctctgatcaaaataacaagatccagtcttagttataaattggggaattttcatcaaaataaggagctactcgttgcataagaagactagtacaacttaaagccaatttaatttcaatgaatgcatgatcagctccattgccaattgagtgtttttcttattcatcagaagatgggttcatcatcgtgtttcatatcaactgttctcaaaccatattgcccatttaaataaatatagatttgtctcgaaattctaaattcatgtcatatttcataaatagcctatggtcctatttattactttaaaatattatagatataatatttttattctaaagtaactgtgttatacaaccaaattattcatttaaatatgtgactttttaaataagtaaatgacttatttaagtaaagtcattaaaattttccagtctgtccttcatccacctgatctttgaatgagttaggaacaatacaggaaactaatacaaacttaattttgattacaaaagatgaaatcattctgttatttattcaacacactatgtgtcaataaaatcttatactgtgaaagaattcgtctaagtccatttgctgttgcttgtaacagaatacctgaaaatgggtaatttacaaagaaaaggagtttacttcttacagttacggaggctgagaagtccaaggttgaggggccacatctggtcagagccttctcccatccaagtactaaccaggtcgaacctcacttagcttccaagatcagataagagtgggcgcgtttaggctggtgtggctgtagacttgttagagcctttttgctcatggggacacagcagagccctgaggcagtgcaggacattacatggcaagaaggctgagtattctaatgtgttcatgtctctcttcctgttcttataaaatcatgaatcctactcccatgataacccattaacctattaatttatgaatggatgaatccattcataagggcagagccctcatgatgcaatcacctcttaaaggcacaatctcccggtgctgccacgttggggattaagtttccaacacatgaaatttgggggacacatttaaactatagcaaaattgtaataaaatgttatatagaagcaatgttcttactgattataattgttatattggtaaagtgttaagtcctctaaccaagggatatatttcagcttattataatagttttaaatttacaattcaatatgaataacatctggtaaaagttcttttcaagaaatgggaaaattagaaatgtttagaagaaaataattcaataaatattaagttcaaactggattcatagtttatgtgaaattctgggaaccaattgcaaggggagaaaatagttacaatagcaatggtgaggatgagaataagagcaggtatcaacgttaattgagggtgtgttatagttctaatcgtgctatgcccactacatgacttttccctgtgtgaggtttccgagcttcttcgtagtaatcctaaattgagctggagagaggctagggtaacttactcacgctcatagagccatagagtagtaaaacctgtatttgaactctggcctgtctgacatcattctgtggtcttttaaaccaccactgcttctccatattaaaactccaaatctaggtgaaaagaagaaaactcagaacatgttctgcaacaaaatataacaaaatataatgtatataaacacttatacataatatcactaatatctttactatgaaaagactctgatacgaacattttacataattcatgcagaagtgttaatcacattgtctgtgatgagctgtgtatgtatctgataaaattctggcaaccagacatcaactcgtaggcatagatctgtaacactaaatatttgcctcgagaaacttaaagaaataaagacaaatgaatgaataggaacatggaactgagtacaagataaaatcctcctaaagcaatcgatgtacttgctgctgcgttattgttctaagcaaaagaagcatggcgaagggagatgtgaagctaaaaacagaatgcttagaaggagatgatagcaggagggaagcaaagatgggaccaagctcccaaaaggcgggctttgaacaaacaaaacagaaagctaagcctttgacggatgcacgggatgcaagaaactttagtcaggaaagaggaggcgaagaaaaaccctccaaagaaaaggtgaacaatattttaataggcaaattgacagatagcaagagatatataccatgctatgttttctcattgcagctgaagacaaactggggttatttatgctttgaaaaagcgtaaatctaaaaaacaattgtggaggaagaagcgatgaaaacacgtgttaatacagaaaacatggctccaaggctttaaacttccttgtgagataaatgcatttacattttccgtagtagctaatatatatatatatacatatatatatatatatctgggaaaataatacacagtgattttctttctttttttcatctacttatgtgagaaaaaagtaggctatctgaaagcttttcagttaaatgaggaagaaagttaggtgatcttgtaaataatatatatgttcaagataatgtaaggcccttgtgtagttttcaaaacttatctttaatagcagtttcttctggggatggggtagttcaaagttgaaatgttagaaagatgttaactttttttcctttttacttctccctttcaggatggaattaacaaatttgattacaaatagatctcagagagaggcaaatgcattgaatccagaagtaacataaaattagatcatgtttagttatgcccgaggtcacatggtgataaaaatgaggataaactgaaattgtctgtgagccagattagtttattttatgccagtcctaggaaaaagacacatcatggtaggatacatcctttttttttttaattatactttaagttttagggtacatgtgcacagtgtgcaagttagttacatatgtatacctgtgccatgttggagtgctgcacccattaactcttcatttaacattaggtatatctcctaatgctgtccctcccccctccccccaccccacaacagttcccagggtgtgatgttccccttcctgtgtccatgtgttctcattgttccattcccacctaagagtgagaacatgcgctgtttggttttttgtccttgcgatagtttactgagaatgatgtattccagtttcatccatgtccctacaaaggacatgaactcatcattttttctggctgcatagtattccatggtgtatatgtgccacattttcttaatccagtctatcattgttggacatttgggttggttccaagtctttgctattgtgaatagagccgcaataaacatatgtgtgcacgtgtctttatagcagcatgatttatagtcctttgggtatatacccagtaatgggatggctgggtcaaatggtatttctagttctaggcccctgaggaatcgccacactgccttccacaatgaacagacacttctcaaaagaagacatttatgcagccaaaaaacacatgaaaaaatgctcaccatcactggccatcagagacatgcaaatcaaaaccacaatgagataccatctcacaccagttagaatggcaatcattaaaaagtcaggaaacaacaggtgctggagaggatggggagaaataggaacacttttacactgttggtgggattgtaaactagtacattcttaacatcaatttattcctaaaagcaatgttcatagggcacactgtaggccatagatttgcctcacaaatttaaaggcctaagccctcaacatgcacagcagtatactcagagactatttgtaaagatgacgattctggaactttttaatgaccccaatcattagcaatgattaaaattaatattcaacattctatatttaccaaggcaataaagtagactaatctattttaaaagggttttaaaatgaagagatgaaacaaaccaaatgattttgatttaaacttcatgaaaacataagttgcattaatcaggtgattttgttttatgagcattctgattgaagtgatcatatttagccccgggagaataagagaaggtaaagtatgggtatggcactgaatttactgagatgattatattgtttgagttaaagaacttgtattaagaaacaagtatgtgccaaacattgtgctaggagcaagcaatgctaaaattacatgggtagaaagagagaatgaaatatctagaatgagttagaaacatcagtgttttccaatgtggagccctgacttcacatgaaaattctcattttcaaacaaggtagtttatgaaaactggactattagcaagacagggtgggcatgccatcagtatagtacctggtgtaaaactagaaattttaatcatttgtgctttcattttataatcagtaaaatccaaggtaggacaaacttttactttttctgtataatggactgatatttgaattatacccaactttaattttttgccagaaattatgctttattgtttctctaaaatggtactatagatctttatttatttctatatatttatatgatttttacatatatgtgcatttacatgtatatacatccataaactatatacatatatacacataaattacaaatatgtgtacctacgtacatatatatgcatatatcacgcaaatacaggcacattttcaatacccctttttgatttttttccttgaagagcatagcatctgaatttattatggatttatttttaatttatggtcatgttctttgagtgcttttggtgtttatctggttgccccaaactcgctagcattgtaaagaagatgtgcaaagcctgaatctagactgactttcatattgactttattagtcaaaaaaagtagatgaaaatgtaacagtccgtgttaaaaatgggaataagacagatgttcaagccctagcttcagcagtttttagctgagatttactggaagaaaacattttctgaactgtaaaacatgcaaaatgcctacgtgacagacttcattaacattattaaatgctatgatatagtaaaagaatttgtaaactgtcaagtgctttgtcaacattaggaatttagttattataggtatttccatatacatgttgtatttagaattccctttaattttatacttagggttgatttgtattttaactaagtcactttatatatctggtcccattatacaagtatacttttccttaggataagaaagtgatctttatatatgtttatcaacccaaatgcccatcagtgatggactggataaagaaaaggtggcacatacacaccatggaatactatgaatccataaaaaagaacgagttcatgtcctttgaagggacatggataaagctggaagccatcatcctcagcaaactaacacaggaatggaaaaacagacaccgcattttctcactcataattgggagttgagcaatgagaacacatggacaccgggaggggaacatcacacaccgaggcctgtcgcgaggtggggggcaaggggagggagagcattaggacaaatacctaatgcatgcggggcttaaaacctaaatgacgggttcataggtgcagcaaaccactatggcacatgtgtacctatgtaacaaatctgcacgttctgcacatgtttcccagaacttaaaatttaaaaaactttaaaaaaagaactgtagatactgatccaaaaaaaatgttcattaatgggggttaaatgattatttctaagtagactactcttgaacccttgaatctttaagaattttctttgctattgaagccattcaaactctattttattaaagctgtcgttattctagtagattttaaacagtaatacctgaatacattagaaatatgcaaatctgcattacatatggcatctgcagagcagaggagtttggtcatctggactcatgctaaagtctccgaaaaatccgcttgtcttaatgatggttgactcgctaatgctatgcgtatatagtcttattttaagtgattgaatgatgtggctaataacccctctgttagatgcactcagaacctcacctacctgggtcctcagctctccagtgaaatctctactttaagtttattttctaacatggtaagagccttcagtttatgttatgctcaggcccgtcactgtgaataaaatattagaaatggactttttttttttgtatttttttaatggatcccttggaactttaaaaaaattatttatttgagctttctactgttatcacagtgtctcctaagcatggcctcccgttttttgttggtaatataattcttacgttattcaaattagtaaccattatttttctcatggctagaattctggaaactattaggaaatcactgagcataattgaatggctgtttatttgaagagctatgtcaaggcagcatagagttgtattttcttgcaggggctctggagtcaaagagcctgggttcaaaccttggctccaccactttctatctgtggggcattgggcgtgttacatttgtgaaacttttgtttctccatttgtaaagtgaggtttgggggatgattaaaccagataactcatgtgaaatatttaatggaaatgtatttggtaggggatttattatttttaaatttggattgcacatgacacatgtcagggatcatgctatgcattttggatagaaagatggctaagatatcatgcctgactcttaaaaacttacctaatggtaaatgacgagttaatgggtgcagcacaccaacatggcacatgtatacgtgtgtaactaacctgcatgttgtgcacatgaaccctaaaacttaaagtataataaaaaaaaaaacttataatcaactgtagtagaaagagatctgaatggcttgccatttagctaggcacatggtatatgtgcttaattcatactagcagccactacagttgtcatgattaataatgagcttccaactgcacagaatgcttttaatccatagaaaatcaaatcagaaacaagtttttgtaaaattaatgtgaaaggagcaacaattaaaatgcaagattgacatttattttctaaattggttctattttctttcacatttacaaaatttataagaaaattctttatttctatgtgatataaagaactagaatgtactttgatgtgaattattgttgccagtgctgttcaacttttatccataatttactaagcacctacatttagacaaaggcattatccatccctttggggaggatttcagatgattcatacacagacctggtctcgaggaatttaagattttctttggggagggaaataaggactttaaccaactcaagagtacttagagaattttctgaaaataattttatcaatgaaaacttgttatattaaaagaaactgtcattctgacttccacaaatctaggcttgaaactatggataacgagatattttctattactctcactcacgtcattttcacaaagtgaaaaggtacattttaactagtgaaagaatagaggaaatggaagtagctcgaggcagtggacgatgattcaaaaagacagggccctattatttgatcaagttatgcaacgactctgggcctgtttcttcacctctggaaggaggaataatctccaagccctttcagactcttttggtaattcacctccagcacatcttctaaatgccagcattaactgtcctctgatttgtctcatgtttttctagccccatgctctcctgttcgccatttaccctcatgcaaggtacaaattacacccatcatcacaagacacttgctcaagtcccattgcccccttgaagacctgccacacctactctctcaaaaaccatcatttcctgaaagtcctatacagctcatttggtatttacagtgtactgccacaagccactaagcatcgttttgtgaatacatgacttacagacttagcttgagtaaagatacttgaaaatgaacaccatttcttggctatcttcctattttgatgtacccttcaggcctatgaattttagtataatagataaccaataattatttcttggttctttcctgcacatctgaataaccctatgcaaagtgatagaatgtttttctataaggaggtcctacactggagattgtgtatttcttaatgctgttgaaggaagagatgtgtatctaaaataaatagactctaacaaacattaatttatatttctattatctgttttgtgtattgagatatctcacaaaaataactaaacattttggcattattgatattacatatttgccatgaatatttgtaaatgaagaaaaatatatatacatcagtaattatcttggcaaactcttcaattatgcaatattgttacatagattacatatctaagtgaacactggagttttaacaatattgtgtgttcataaatgttttatttattattgccactaattcttattgccatttcaagaactatgtataagttgttctaaaaactattaaagtataggtgaccatggtcactactgcctactttggtaaaggccaaatatgtgaagactttttaatgtgttaacaaacgttgaaggttttttaacctgttaacaatcagtaggactcttgaaattatttcctaagagagtaaattttacaacttgcaaagcatgattaacctcttgtaattataaaccatctcttgtagttatgtagcattttgttaatgagcaaagaaccattgtggttcctttttacatttcttaaaataattctccgtaacctcattgatatctccagtaaatttagataagcttttttttttaaaggagggttaaaatgacattttaaactaatttttcttgttagttatacagagttgaactatctgagggttttattgacagtcataaaaaatttgttattttctgtgaaatatagagaatttaattcattatcatattattaattctgtgggccattgtcttaattctagaggcacaagctgttttcatcccactgaaatagaggaatcaaagtatgttccttgctcaaagcacaaaagtgacatactacatagtatgcttcttgagtagtcgtaaatctcatgtgttaaattacatcccaaagatttcagtatgttttatgactttaataatttatggtaatttctaatctggcctttgttgacctgtcttgctttttaaatttttagtttttcgacaaaataattaacatattttaataatcttccaaaggtgtttaaaatggcattgtatagagatagctgaaggcttttgagcttctgtgttgtaaacactttcttaataaaacatgaattgctaccagatgatccagcaatcccactactgggcatttatccaaagaaaaggaaatcagtatctttgaagagatagctttgttcccatgtttactgcagcacttttcatactagccatgatatggaatcaacctaaacgtccatcagtggatgaattgaaaagaaaatgtggtatgaaacagaaattgctgctttaatttatattaaacacactcatattcttctcagctgttaagtattgagttatagatttaaagaattctattgtgaagactaaagtgactattaaagtaagaaattattttttccattatatttaacttatttcatactttaatgttagcgccaatgagcaagactattgaatacaaaaactaattaagtagtggtgatagtacagtatataagggagaacattcttttagaaaggaacaataacagggagcaatagaaacaatgaatgagtgtaaggtcacttagtgttaaaacagctaaaatatagtacaaataagttgcgttttaatagtgattttatataattacaccttgatgttttatttgttacaagaattgtccaggaagatttctctaaagaccaaaggcactcttcccctaaataactccaaagccagtcctgtgtttctataaaaaaaaaaaaaaaaaaaaaaaaaaaaaaaaagtgaaaataacgtgatgaacatttttgaggaagtataaaaccaaaatactccactgcatagctgtttctgcaggtattgtattgatatattacattattcagctttggagtctccacatccaatgttacatcatcactctaaattaaacatgtatagataaatgaaataaatgagatagcatatgaaaatctcatagcccagcccctgcactatttaaaatagaaataccaaagaattgtattcctcatctgaaagctatttagtggtggtgtttcaaataaaaattccatctactgctgttgcttccattgtatctttttctctgcggtactgaaagagaaagagacccagaaggggccttgtctgaagtgtccctcttttaagctgttgctgctttaagcacagggtggacaaatgtaataggagtttcataaaggtggaataaaccagcggattacggtgtgggtgaatactttcagatgttaaccaggagctctgcttgcatgctgggagttgcccatgcctcttctagattgaggcacattatcatgcacaacctaactccaagaaatcttttaaaccactggaaattgaacccagaacatgtctctaagccagccttttcatcctgacaccgaatcatagcatgagccagtctgtcagggatgctgctgctctctaggcaaattttaaatgttgaaataatgaatcatgttttcttgaaaaccatgtacaccaaagaaaagttagtcattttatagatgatgaatattaacattttcttagacaatctgataaattatcagatctcacttttggctctttttaagacagttatgcctcagaaatattaataaacccccaagcccttatactgatcagtatgttcactactagctatgagaaattcttgaagttcttgtaattattgtattatttccttactttcattttattagtatgtgaataatatttttaaaaattctagtgtatgtcttgtatatattttaacaacatgacttttaattaatgtcttgataacatttcttctagtgtatgttttcagtaacatgattattaactgtaactttaaaaacctgtggattagatgggaccattttaaaatgttttaaacctggaaaatctgatggctttaggtttagttcaagctatagatcacctgtggagaatggaactgccaaaaaaaaaaatagctgtagcagccctttgagtattctaaaatagggatgttatccagagcattggtttctaaagcttccattatttattgatgttgagctttcaggatttagctacaatatttactcaacatctaagccatgcttttttatcagtcatgttttatatcttttataatcaaactgcttatcactgaaaaaaatatataagtttctatgtatctggaagaattctctggtgtttcttagatatggattttgatgtgtggaataagaattcaattcaaggataacagagatgttgtcctgaaaaaaatcgaagaaaatcagcttttctttaacattctgtcaaagctcctgactattagtttatcagcactgttttgccaaaggtgtcttctcttctcttctttgaaaaaaatcatctgctgctgctacgccgcaagtgtgttcccgctgtgcctgagaagatgtgtggcataaaaaaatgggcatggcctgagttaaaagtgctacatttaagccagagctggcttatttattagttgtctaatcataggaaaatgacagagcatgcttttctcttgcaatatccgttgctgaaaattaaacacatgagcagagctttcagagaggttgactggcctctcagacagcacctcataggatggcctgtgttgaagcatctcctttaaccagggtctgtccctcagcattgggttggctcacctagattggattgtcccagcagaaaaaaaaaacccaaaattcagaatcatatccaaaccggaatactctttcattcacattacttgtactaccttttcagaaactggatacctgagtgtgtgagggtaacttagaaacttatctcatggttagaagttttagaattagagagcgatgatcatgaaacggacttcatgatcagaagcaatggagcaaggaatgagatgtctttgaggagtatttccctgaggctgtggataacgctgacgaataatccccaccttaaaagtgggttgaccactctagtagctgtaaggtgggagggttctttcttcagagataaatctgtgctcttcacttgcccatttcccaggttttcatgtaggtagaagaaacacctgtaatctgaagacactcttccttcagctttgttagtgacagggatttaaatatgtctttcacacattttccttagatagttaaatttcacttttcctgtttgtttttctctgaaggtattctaactcccctcctaatggacttctagagctttctaattctatgcaatttctgttgatttgttctggtaaactttgaaggtaatctctgattcaacttcttggagattctatcatgtcatctctgtttattaactttatgttactcatggtttcttgatgaggactcattaaacataatgtaagtagaaaattattaactacataatatttactacgggttgttatttctgatagtagctagctgtaagattccaattgttcttcaaatctttgtctcagtgatctctgtgtagttcttgactacttcaaataacttcctagaaggatagggatttaataatctcttaataggaacacttaacacactgctggtgggaacgtaaattagttcggtcgttgaaagcagtgtggtgatttctcaaataacttacaaaagaattaccatttgacccagcaatcccattattgggcatatacccagaggaatagaaatcattctaccataaagacatatgcacgttgtgtatgttcattgcaacactactcacaatagcaaagacatggattcaacttaaatgcctatcaatgaacagactgaataaagaaaatgtggtacatatacaccatggaatactatgtggccatgaaaaagaatgagatcatgtcctttgcagcgacatggatggagccagtggccattatccttagcaaacttatatggaaacagaaaaccaaatactgcgtgttctcacttataaatggaagctaaatgatgagaacatatggacacaaagaggggaataacacacactggggcctactggagggtggaacacaagtggagggagaagatcaggaaaaataattattgggtactatgtttagtacctgcgtgagaaaataatctttacaccaaacccccgcaaaatgcagttcacctgtatagcaaacctgcacgtgtacccctgaacctaatttaaaagttataaaataaacgtatcttattttcagtacaatacaccacagagtagaagggttaaaagagattgcttctgaggaggtgagatgggggtaaggacagcacaagagcattttggggggtgatgaagctgttctgtgtcttgcctgcgatgatggctacacgactaagcccttgtcagaactcacagaactttacttcaaaaggagcggattttactacacatcaattccaataacaaatactttgtctttaagcaaagggatacctaaatatagcgtattgaatggatctccagaaaaacacatttttcagttcatgtttcagcctaggcctcatctcatccaggaaaccttgtcttgcttgcctttacatacatgtggcaatcagtagtttcttttagggctcggactgaacactcaatgaacttcaatcttagcgcttgtcgtagcagattgacatggtttatttatatgtgtcattctctgtagtaaaaggaaaggatcaaggccattcacttttgtagtgattgtgcatggcagtatttggcacatagtagattattaattatggaacttctgttttcacacacacacacacacacacacacacacacacttcagagctattttcatttaaatatttgctttagtctccaaagcccctctgcctcaacaccaacccttctatctcattattcatcagcttttctcctattacgaaactacttaggaaagcccacttatttagcttatgatggcaaaaataaatatttgtacttttttttttttttttagtcatcgcttcatagaacagcctctgtcctctgcttatgccatgtctgaatatatgctggaggtaaaaagagttcctggttgagagcttcaatttgagaaactatctgagattactttccaggttccaccgtggaacctgtctgaccttgaacaaatgacctcgaacaagtggctgaaatctcttctatttcgtcaactgtaaaatgggggaaaaccatgtctatctcatggggttcatgtgaaggttaagaaattgcttattcagtgtttagcacagtgcctgatatgcataaagctcctaggaatattagctgttattgtatttccttaaagaagcccatagctctatatgccctttcattatatgttttagtagcccaatttaacatatggataaaatatttttaagttaaatgatttgctaatggattgttgaacgagtggcagacacccatattatagacgaaggtcaagtccataacatacagtacatttccccactttcatttcccattaccaaaattcattattctcctgagaaactcattatagaattcatgtcagattcatctgtgtgttcccagcagtgccttatatccagaaataacactgagtcattgtctagatgtagcagaggtggaatcctccaaagagaagcctcagagtggccaggtttgccaagtatagggatgccttgattactggccttactctttatgctcgtgaattcctaagttttattcctcctgtagtcatagattggcttttaagctacaagctgaagagagagaaaacctcttccacctcgttggaatatgtctcttcaatccatttgagccaatttaggacatgagactgctcttagtctagaaccagtcatcaggagaattccaggtctgattgactcggactagcgggtcaatatcagggcaaaaattccaacgcacaacacgatgtatcagtaaggagaacctcaaaattatttcttaacgtccagatcatgttcctatttttatatatctattttctcacataagtcattaaaatgatgtacctgtgcgggtcctttaatgatactcaaagatcttgaattataggctaataactaacttaataagctgcagaaattaacatttctgctacgtttatgtagcattttcccacatgtacttcagaggcttgagaaaagaccctgaaataatgactgaataacagctttactcacttaatttcaaatttgttaattcttctgggaaataccgtcaacatccattttattatttttctcaattacatgtacgtttctacatcagtggataagttaaggagaagaattccctcatgataattttttcatgctcgaaaattttgaatcaattttttattttacattatactctttcctagtcattagaaagggagtggtggttaagataggcaagaatgctttataaggatactactctcgtttcaattcttaacatcaaaaaccttaacagtgtgtagactataaaataaaatatctagggatcagagcattgtgctgaactttgcaggttttttagtcaataatatatatgacgtgttcacagaattctttgtcaacaaagtacttttggagctccaggccatttaagttggtttttgtactttttctttttcttcggaagactttttttgttctatttacctggaagtgtttcttttttggtactgtgaattaaaatgagaccaatctactaggcaggaaaaaaccttaattagattgttgacacagacaaataagaatgtcaattagcatctactgtcacatgcctctccagactgcttctaggatgagtggcctcaagcagctacatcatctttatactcctaaagcatcaaggaaacttggagtgacaattcatatcatgaacacatccacagtgatgatgattgtgcttcttcccccccacccaacaacaaaggatgaatgccaattaatgtattcagttttttgcgtcaaaggctggatcacttgtgcaatgagggtaatcatcctgaccagacaggccatacaatccatattgtgtgaattaaagataatatgcgtgaaacaccttactctggatgtggttcatagcagtagcaaaaagatgaaaactatggtatgctaacattttagagatctgtactctattttaaataattttataaaagtgcatatacaataaaaagtgcacgtatcacaagtatatgcctcaaaatctaaagccagtcatgtaatcagcatccacttcaagaaagaaaacaaaacagtacccctggttcctctttgcaatcattagtctcccaagagtaatcaccgatctgatctgtgacagcatagattggttttgccctactatatttttgctgaattatacaatatatgctctttaatgtctggcttcttagtgcattgtatttgtgtatcagctattctcttgtgtgtagttattaaacaatcattttatgggctgcataatattccatagggtaaatataacagttttattgataacttagctattacaaatagtgctgttgcagacatatattctattacatgtcttttggtataagaatttacacatttcacatgggtgtatacccagaactgagattgctaaatattggggcacattgtatacattttgatttagtagataagatattgccagatatcgtaaatgcacagtttgataaatatagagatttatactttttctagagaaaagccatcaatatcagtgtatgtgtatatatatacgcgtgtgtatatatacgtatatatatacgcgtgtgtatatatacgtatatatacacacatatatatacgtatatatgtgtatatatatacgtatatatatacacatatatacatatatgtgtgtgtgtatatatatatatgaaacaactcagaagcagaaagataccccatgttctcacttataagtgaaagacaaataatgtataaacatgtacacatggacatagagtgtgtagtgataagcattggagactgaagtgtgggggtgtgcaagggaatcagtgataaattaatggctacaatgtacataatttgggtgatggatacactaaaaatccaaagttcaccactatccaacatactcacataataaaattgcacttgtaccccttacattcatacaaataaaaaattatttaaataaaaataaatatgtgtatatgtatgcatacatacatatgcatatacatatgtgtttgtgtgtgtgtatataacttacacttaaaataagcatggatgctgcaatgaatgctcaatttacaagggttgtccatccaaacttgtggcaagtatctcacctctcaagttgttttcttttttcttcatatatttcttgcttttgtctaggaaggaataatttggcttgcctttcaagagtgtacagtcagcatgataacccaaacacttaagacacgtgctaacccatgtggatcccttgagagaaggaaaacagtggtccttttactgggcagatagagcccggggccaggtttcgtggcttgaagatttcagcttctctgcgcctctcagctcagtgcctctggaagcaatttacaacttgtgaggccatactcaaaggccctgttattaattccccgccttccgagaccccatttcagaggatctcaattgctctcagagtgaatttactgtttcctgaattccgtaatcccaatagcaggtctgttgtcctcattagatagcttaagttagagtcggcagtgtaattggcaactgagctactaagtatccaatgcttatgtggaaaatatgttccctattgcaaacaactgatattcatattcaatttggcaccatcatctatctataaagcagatactacttgtgtttattaagttttatcccaaataattattttagtaataatgcttgaaaataggccttggtcatttgcatgtctgtatatggcatatcctgagtctttgtatgtattagaaagatcactcgttttgacttgatggtttaataaaagatgtccctcactttgggcagagacatttgaaaaaggcactccaaccagggacctaagaggtgaatgagatgcagctctgaatcaggtcacacggcctcaggaaggaaacatcttggttttcacatccctcacttctcgatgtcatgtgcaatacacaaatgacccctcaacacacacacaggcacatacacaaacacacactcactcactcactgtattgtctctttccttgactaagtccttcttactaactcaagctctaaagcttttttacttacctaaggtgagtgtgtgaggatttgaggtttcaatattaaaattcagaaacatttaaagttcattttaaatattagtaaaaaaaaatcttgacaaaatacaattatagacaaaaagaaaattcagaatatttggaatttaaggttgaggttacagccctatttatgaaatattagaagaaaaatgctggagagaataaagcaggtttatgagtctgatagaaagcataaccagatgattatgcatatatttgcatatgcaaagctttctaggcaatctgaacatttaaacctacaaatgtggctgcgatgaacagccacagaagagcaggctagaacagaagaggaggctagaacagaagagcaggcagaagttgtaaatgaaatgttaattttcaatggttgatctcccaagtactggaacagatttgtgctgttttcaaggttttggttcaaagaatccagtagtgtattgaattgttttgtggcacttccctgttattttgctttgtaagctacctcaatccatgaagtggctatgagccccttatacaacactgttgatttttttttccttatctacgcaaaagatttttgattcagggccaggcatggtggctcacgcctgtaatcccagcactttgggaggccgaggcaggcggatcatgaggtcaggagatagagaccatcctggctaacacggtgaaaacccacctctactacaaatacaaaaaatcagccgggcgtagtggcatgtgccggtagtcccagctactcgggaggctgaggcaggagaatcacttgaacccggtagccgagatcctgccactccactccagcctgggcgacagagccagactccatctcaaaaaaagaaaaaaaaaaaagattttttattcaggtggctatcagactcattaaatagaagccttaggttaagttcacgggttgctagttggaagcctccatggactatgttcataaaataatagaaaggagttatgcaggacttcttgaaatgttatttaaaaagtcagaataggctttctattacttgtctgaggtcaaatacatgtagtgctttctgaccatttcatccagggtgttagctaggacaataagaggtgcttaaaaattattagattgagtaaatgagaaagcccttagaaacataggaacagaatgacccttgctttggatctaatattgactcccacgcctaaatccctttggagaactcctttattttctcttccatcaagagcaggtataaattaaaaacaccattaaaggggccatctagctcagctgaagctttcatcacacatgtaggggaggtatggttgggagggatctttttatcctttaggtcttcaatttacataggacttttgaataatcaaatagccccaaagagctgatcttaggactagttgtaattgagactatttctccatggggtagaaaaatctagttgtaggaaaactgagaagtagatgtatgttaacctcaaaggctgttttttacaaaggatgttaaagcatcatctttgctcagaaagggagcaataaaacaaatgagtggaaataacaaaaggaaataatggccaggtgcagtgcctcacactagtaatcccaacactggggggctgtggtgtaaggatcgcttgaggctagcagttcaagaccagcctgagtaaaataggcctcatctctacaaaatagatagatagatagatagatagatagatagatagatagatagatagccgggcgaggtagtgtgcccctgtagccccagctactcaggaggctgagatgggagaatcgtttgagcccatgaggtcaagtctatggtgagctgtgctccctcctgccactgcactccagcctgggtgacagagtgagatcctgtctcgaaaacaaaaggcatactttttagatgtaatggaatagagtacttccaaacctggctgcctgctggagttgtattggaagaggttgcacgacttcagtggagatggcctagatgcctgctcagcagtcatctagttaaagcaactaagaacatgtaatatgaaactgcaaaaagagatcgtgtacgtaaaatcactctgggctcctcagatagagtaataaacacaactcctgacagccaaataaaaagagaaataatacagcccttgacttccttggttgctttgacatactaagtaggtgttacaggttgggttctctgggaaacagactctaaaacatttttatttttactttatttgttgttattattattattattattattttagacagaattttgctctcgttgtccatgttggagtgtaatggcacaatctcgtctcactgtaatttccgccttatgggttcaagtgattcttctgcctcaaactcccaagtatctgggattacaggcaagtactaccacgcctggctaattttgtatttttagtagagacggggtttcatcatgttggtcaggctggtctcaaacacccgacctcaggtgatccacccacttctgcctcccaaagtgctgggattacaggcgtgagccactacgcccggccagactctaaaataaagtttaatatgcagaatacttatcagggaatgcccactggaccaatacatattcaagagagggcttagaagcaggattggacagaaagagaagttgagctgtaatgcaggcccaataacagccttagtgttaagcaggctgagagattcagcagttaatgagacagtcaacccaaacagttttataggcatcaaaagtatgatcagcatggtgtcagtttcctgtgtcacttgtcccacagtatgataccaaaattaaagagaccagatgacatgcaacacaagcagtgtgcactctgttgttgagaagccaatttcgtcatgcaattaagcagttttatactctgcagctgtactttaaggggagctgagatggaacatcatatgtctcaccataaccagaaaggcagatgagaaatgttctatcgccacctcccacaaggtaagggacttccctaaagatacagaggtgggtggaatattgccttggtagacttcctctcaagactgcctatcttcccatgttggaaggatcacagagcatttgtcaagacgtgggtcaatctgcagttgaactttgtgtatgtggcctatgtggatacttataatatcattgggcacctccatagagctgtttcccaattgaccaaacatatgggaagcttcagagcttcgaatgacccttcagagtagtcctgagaacagtgagccttactactcctgcattaatcagtcattggatgatagccttctcagaaataagtcatgaccttgtgcaagggggctcttcatggctgggaccacccctaaaactgagagctgaaggctgtctgccaccagcccttccacctgctgggacaagttctttattgaagggaaatctgagtagttcatcagcgtccatcacagtagtcaagccgttcattcttccttcttatgacaacattgtgcttattgttatgtaatccctttccagaacattttaggttaagttttaaaaataatgcatataaatagacaattcaaatactggggaaaaaaagcttgcacttatattgttatagaaatgtgcacacttaaagagctgatttcttctgggtatttacataactttatttaaaaatccatccatttttaattagctgtttttaatatgcagttagctaagatattataagccatatattaggctaatggacatttaacagcttagttaagttcttttaatggaaatgctgacaaacctttgtctgtaattatagcaacactgtgattacagaaggaggtgcctctccttgttgtttgcagccctaaaattccatgtggctataagtaacaaagtccattattagataaacacaagtcatacttggcattacttgcattactcgtctccttgctttatttgaatcattttttaaagttgtaaaatgtttttcaaaactcagaatagtggccagttaataatatgattcctcttatattatgagattttaaaaaatagttcaccagtttctggtggcctctatacccattggcaagtcctagccattgtgaattaagtaaacaattctttatggaaattttttaatccttaaaccctataagtttttattcatcatgtcaggtcacttgtcaaagggtttaacattcagaattcaacaaaagtttatcaaacacctattacaggacgtgcaattttgggcgcactgggatttcagcaattaacaatcaagatatgatttgtatcgacatggatattacattctctcacaggagacagaaaacaaaataactagaaaatatacataaagagactttaaaatggggtaaaattacagattgtgacaggatgaccactttggttcagaatatctaggacatttttttctttttttttcccctccctccctctttcttttttttctttttctttttctttcttttctttctttttctttctgcctttcggagtcttgctctgttgcccaggctggagcgcagtggtgcaatctcagctcactgcaacctctgcctcccatgttcaagcttttcgtgtgcctccgcctcccaaataactgggactagaggcatgcaccaccaggcccagctgatttttgtatttttagtagagatggggtttgaccatgttgcccaggctggtctcaaacttctgacctcaagcgatccacccgcctcagcctcccaaagtgctgggatttacaggcgtgacccaccaggcccaagcaaggacatttttttctgagccatgttatttaaacagagatctgaatgacaagaaggggccagctctgtgatgtaggggaagaaaaatatgttccttctacccttctaggctgcccagctggagtcctacaaagttagagtgacaaaagacagattaacaagaggaaaagcctagaagtttattaaaatattcagtgcacatacacctggtagaaactcagtgatgagtaactcaaaggggtggttagaatgttgggtttatatagcatctgaacaaagaacagtaaacttgtagagaaatgacaaaacaaagaaaaaaggggtttaggtatttagggttgccaaactgtaggaaggtaaatatatgggagaaacatggagtatagtttgtttatgccaagtctatcttgagatcaacttttcgtattcttcatggccataacaatttcccaggagagagggcttatagcagttatcatttctcagaagtttctgcttttatttagacaagggaagcactgggaaggcttctttttgcttatattgattcttacttgcctctaactaaaagtaatctttatgtcaaagtgccatattttggagtggtatatattgatctcctataataacaatcaaaaggaacagtattctaggcaggagtaccactaatgcatagtgtttggtgtaaagacaagttaacatattcatggggcaacaacaacaataagccaatatggctaagacattgaggatgagtgagttggagaagtaggcaatggccagctcatataaagacttgttcgtttttataaattgtttagattttattgtaattatggtggcaagtgattggagagtattagcttcactttgactggcttatcgaaaacggaatgtagggggtgaaagtggaataaaaagaccagtcattaattgagtagtccgtgtgagagatgatagtggcttggacaaggacgattgtactggagagattgaagcgactgatttcagatttgtagtcaacaaggcttaattggtaggagaaaaaaataaatcagtgttaactctttaatgtttaacttgaataattatgatgagggtattaccatttattgagatgtagaatattataaagtaagagcagatttgttcaaaaagtatcaagaatctttatttggacatgctagtttggggatgcttattagagaccctaggaaactgaatataaatgtggattttagagaagagcttagggctggcagatgcacattaaggatctgtctagagccatggcgctagagacctccaggagaacataaatagtctcaagatcaagccctgagacactcagatgtttagaagtggaacagaagagggacatccaatatagaataccaagaattaggaggggaatcaagagagtgtggcaatatgaaagatacaaaaagagtgttgaagggagggagtaattaataaccagcatgttatgaggggctcagtataatgaaaagataagtgactattggatttggcaacatataattttttggtgatctggacaagagcaatttgaacagaatgatggatatggaaggtccagaggagtaggctgagtaaataatataaggtgggaaaatagatacaaagattatagacaactttttcaagaagttttactgtgaaggggcacagcaagctgagacagtgaggataaataatagactcaaggatggtaactttagaataagaaatttcaatctgatgggatttaagtgttagcaaggaagctttaagaagttattttccccattagaatgatctgaaaaatgttttagaacattcctcttatattctattttatcacatttatataactttcagagaattgaaagaggtattaagttattatgaaattttctgagattaataagataacaattataggatgttttcttttagttgaaatacacctactcagcctaatttttataacttcttactgaagtataatatacttcagtagaaaagcatgcctaatataaaggtgcagctagatgaatttgcacaaactgaacacatccctttaaccagcacttagattaaaaacagaaccttgatgatacctcagaggcccccttctgccccttttcagtctctccgtgctacccccatggataagcattatcgtgatttctaataccatagattaattttgccagtttttgaattttatgcaaatggatctatttcacctaattgtaaatatataacattgtcatagcaaggcactcattgccttacactgaaaattacattgactctttgccacaagcttagacttgctttctcattttattatcatcaagcctatagctttcacactataccttgttcctgctcttccctactctatttcttggtagatattctatatcagtcttagagtgcagtttgcagaacccctccatcagaatctcctagggagcttgttaataatgcagattcctaggcccctcccatggtttatgaatctgagagtgaggcagacaagactataccctctcatgcctctataatgtaataatgtcttcctagaatgttctttgctgcatctcttattaaagaaatcttatgggccgggcagggtggctcacgcctgtaatcccagcactttgggagcctgaggcgggcggatcacatggtcaagagatcgagaccatcctggctaacacggtgaaaccccatctctactaaaaatataaaaaattagccgggcgtgctggcaggcgcctgtagtcccagctactcgggaggctgaggcaggaaaatggtgtgaacccgggaggtggagcttgcagtgagctgagatcacgacactccactccagcctgggtgacagagcgagactctgtctcaaaaaaaaaaaaaagaaagaaagaaaaaaagaagtcttatgtttcctttatggccagagcacaacattgtcatgaagtcatctaaaatttcccactagaggtaacatctccttcccctgtctagctcttttaaagcattacctccatttgccttgtatcatagctgcttgtacacctgtctgtctttccgctgaggttataatcctctggagggtcatgactttgcattcctttgtgtctcccattagcagccagcacagtgccttgcatactgttagttctaaataacttctctctctctctctctctctttttttttttttttttttttttttttttttttttgagacagagtctcgttctgtcacccaggctggagtgcagtgcaatggcatggtcacagctcactgcaacctccccatcgtgggctcaaatgattctcctgcctctgtcttccagtagctgggattataagtgtctgccaccacgcctggctaatttttgtatctttagtggagacggggtttcaccatgttgcccaggctggtctcgaactcctggtctcaagcagtctgccctactcggcctcccaaagtgctgagattacaggcgtcagctgctgcgcgcatccctaaataaacttttttttttttggcatgaaatctgtaacactggaaagatgttattgccttagaataattaagagattaaatgtagaatctcaaaaacattcatttttttccatgaaaactttaccaggcctcaagggataggaaaattatgggtacagaattgagaatctgtaggaacttgcaagataaacaacggtttcacaagaaagaccttgttggagagttaaattttcagacagttgtaataacttcacattaaagttttgtcaaaaaataagtatctgcatgttttgtttgccttccaatgccctcattttatttgattttttcccataagtaactatagtgaaagcacgaaaatgtgtttctgtgtttgtgtgcctgtatgttaattgtgactgtttctattgcattgttattgcagaacctaggcacgcactctgtaggcttgggtgctttctccaactgaaaaaaatcctacatatggataaattatttttacagccagtgtttaattttacaagtggtccccctccttctgtttttaggatggcagagagaatacatatttacttaccattatcacttactcatgctttgagcttgaaggaaatgagacagaaaaatgaagtaacattaacttctctctggaactatgtttctcatattagagctttatctgaggagttcacttcctctctcttcaatgctttgttcctctccagtcgattcaaatgtcctcttaaagcagaagttccgaacctctttctgtgacttcaggagagcatgagaatgtaaatataagttttaggactaaattttcaaagactttttccactcagctctcttttcctcttcggtttgttgttgtcgttgttgttgttgtcgttgttgttgttgctgctgctgctgctgtttttccccttccacttccgtaactgagctcttagggtccatctggaatctgattgcaattaaaaaaaaaaaagtttatttttacctccttgtacgtgctttctcctaaagcaggagtcagaagccttttttctttgaagggctagttagtaaatattttaggcttgtcgtctttgtcgcaattactcaactacgctgttgtagtatgaaagcagacaatacatacctgaatgagcatggttttgttcctagcaaactttacgcacagagaaatttggatatcgtataatttttatgtgttgcaaagttgtattattcttttgatttctccccaaccatttaatatgtaaatcccattcttagcttgtgtgccatacgcacacaggcagcaaatgcgagttgtcacacaggctatagtttctgactttatgtcttaaagtaaacagtaataatcattctctttttccaaacagtccactaatctccctttgtattcagcccttgcatagtaaacgccgtttcttcatcatcctgatttttattctgagaaaatactgtatattgttcccatgcactagggttcggggaaatttaaaaggatgtaggatctccttttcattggtcctaaaattgcactggggaggcaggtcatgtttatgaacagataaatagtatcataatataatcatgcatttctatggctagcatttagaactatagcttttgatgtcatgtggtttttatatggttgattatttttttcttatttataaaatgaaaaagtttgagaatttttcatctccttaatgtattcccttatttgagggaaaagtatttacctactacataggaatttatcttaaaattttctttgtctatctatttttatggaatataatcgagcaactattttactaattaatactttaatatcattatgaaaatgttctcatatttttaaccttataagatcagataattgctatgccaatctatggttgaaatgggttcttatacttaacgctatgctctttcttctgagatgtaaaaatatgtttaaatcagaatttatataggtgtcaattcaaaatgacagtagttcattattttgattagtataaatgttcacaactaattctattctcttatctattaagtcaccaaataaagtatatttgttttaaatatttaacagtttaaattattctttgaaaacttatgagtctaaagtaagaacaattaacccattcattttgcaagtgggatagttgaattttacttgcaatccagggatttttgacagtttgaaatatacatacataccatgtatgtttaggaaaacatttaaaaagagggggttgtaaaataataatagttcttccatgattttttagccataatgtttataatataaaatatgtatactcttgttattgaatgtagtatgtttctaatttaccagaaggcaagagaataatcctggagaatttctcaaggcatcttcgaactctttgatttattgctcacatatagtaatttgccaaatgacgccctagtgaactgaaagaattaatgccccgtcctaagtcactttcaccgagggactgaaaacctgcagcattttgccaattagaggaggaaacaatctaccttgcagagtcaggagtactggataaaggagctaagagtgttgctttttttccccttcttactttaaaaatcccaattcatcccatgtctttcttaaaggctaagtgaagtagtaagtacgtttttgcaacatacgaatttagcagactggccttgtgtttatttttggccggaaccattacacttatttccaaccctctcctttatttgttggttgataatgggctaattttgaatctttactgtcaaaagaacattaagagaagcagccctgcctgcatcgcaggctatgtctgtcctttgccgagtattaaacactaaaaaaaaattaagaaaatactaacaaaatgacaaagcattaagaaaataaaactagatgttaaaggaaatgagaaaataggaaaggatgctgtacctggagtgattttttttccccaggctacctaagatgatcaaaaaagagctaatttctcttaggtttctattaaggaattactagaatatcgggcacaccaggaaactttatcagtggacctgtcctgaaccaaattttcttaatgtatatatgataatttgttaccacatcccagattattttacaggaattaaaatatatttgaaacactgacagggaaaattgggtaagacattgatagatactacaatctgtacttgaaactgcactcaaggaattcgttagtcaagaaagaacacaatgactgtgggcccctctgggttttggaacctcttttgtaaagcatttttttttttcccaaatagaagatattatttttgaaaaggttaaataaaaaatctttgttcactatatagtttcctcctaaggagtaaattaatttatataaaatattgcaatataaataacaattttaaaatctcaaaagagcagtgttttaaaaataatgtagaaacattaagaaatgacttcaaatgataagaatgtcattggagagcaaagggtttttaatattacatatcgtggcacgtatatcagcacccaaccgctcaagatacagagttctttacaaaaatcaaacagaaggaaatgtgccaccttgttcataaactatatttaataataagccaggcagataaagtcactttcacaaataatgagcaagcccatggtaatataattcatttacaataagatttatctcatggaattcttagactgtgctttgaaatttaaataattctgataaatgccaacagaatagagaaatcaattccagagcaattactaacacgttgcattacctttctaacattaatatttctcttcatacatatcattgaagagaaaatgaggatggaaaataaaaagatcaggtaatatatttgctttctcatctagggttgttatgatcttcaagatgaagttttattttttactcctagcaaatgatattcttttttattttagtttttattattttatttttctgtaaattattggggtacaggtggtatttggttacatgagtaagttcttttttttgatatttctgagattttttttttattctactttaagttttagggtacatgtgcacaacgtgcaggtttgttacgtatgtatacatgtgccatgttggtgtgctgcacccattaactcgtcatttagcattaggtatatctcctaatgctatccctcccccctccccccaccccacaacaggccccggtgtgtgatgttccccttcctgtgtccatgtgttctcattgttcaattcccacctatgagcgagaacatgcggtgtttggttttttgtccttgcgatagtttgctgagaaaaccacgaggtaccatctcacgccagttagaatggcgatcattaaaaatcaggaaacaacaggtgctggtgaggatgtggagaaacaggaacacttttacactgttggtgggactgtaaactagttcaaccattgtggaagtcagtgtggcgattcctcaggcatctagaactagaattaccatttgacccagccatcccattactgggtatatacccaaaggattataaatcatgctgctgtaaagacacatgcacatgtatgtttattgcggcactattcacaatagcaaagacttggaaccaacccaaatgtccgacaatgatagactggattaagaaaatgtggcacatatacaccatggaatactgtgcagccataaaaaaggatgagttcacgtcctttgtagggacatggatgaagctggaaaccatcattctcagcaaactattgcaatgagtaagttctttagtggtaatttgtgagatcctggtgcacccatcacacgagtagtatacactgcaccatatatgttatcttttgtccctcggcaccccttttctaccccccaagtctccaaagcccattgtatcattcttatgcctttgcatcctcatagcttagctcccacgtatcagtgagaacatatgctgtttggttttccattcctgagttacttcacttacaatgatagtctccaatcgcatccaggtcattgcaaatgctgttaattcattcctttttatggctgagtagtattcatatatatatatatagacacacgtacatacatatgtatatataccgcagtttctttatctacttgtcgattgatgggcatttgggttgatacttgcacacacatgtttatagcagcataattcacaattgcaagtgatattctcaggaagcatgatgtaagtgacagagacttactttgtagactgcactcattcacttgttctctgaatgtgctctaggcagcctgagtttctactatgtcagtgttacatagatgagaaaccccatgggtggtttccacagaggctgcaatactatttttgataccaaaaatctgtttggttttgtgagccccagatgcccatatggaaaactgaagtgttgatacctctttgtagccctctgatgaactgcatggttcaccttcctcagcagtttgagcggggtggggagagcgcctgcttcctagccatccgattggcctgaatcatcaaaaatgctatcatgaaacaggttctgtttatctgctccagattacacccatcatgttctagagtgctggtttcatgcttgaatctagatcaagcctgctttcctcccctgcctgtactccctgtggctacctacagtcctgctgctgacagataatctaaaccaatagcacctaattagcctatttgctcatgtgttttttccatcgtggtataatgtcctccttgtcaatttagggtgaaaatgtagcaacacgttgctgatggtttaatttctggaatgcaggtaatgaatgtgtttttgcttatccaagtcttcccatcagatgtcaaatatagaagaacagtgttcagaggtcctaaatttaaattggagtgagaaattcacagcgcccctgaactcaggcaaaatgcactctgacaagtcaaccagatattcacagatggtctggaggatttgaagcctaatttggtgaaataaaattaaatgagtgaaattgtatgcagtcattaatctatcaccatacttaaaatgcttcattgaaatttcttttactgcttcaaatgaaaaaagatcaaactatgttatagaaaagcattcaaaacccttacataacatagataaaacttggttggagacttacagaactttctctgctgcttcgagaaagttacagtgcccacaaatctattgctattagaatattttattgtattcaacactcaattctaccataattatgtatatgagaaaaatatttttacctataaaataattattattaccttttaaaaatctgacattcttccttttttctaaagaaacatatttagatttagcttttattttatttttgtgttgatacatagagattgtacatatttctaagattctagtgatattttgatacaagcgtataatgtgtaatgatcaaatcagggtaattgggatatccaccatctgaaacacttatcatttcttcttttcaatgccatcataccaaaaggaagtaaatagaatttcaaatataaggacagccatgattttacatacatgcctacgattccaccacaaaccataattacgtcccccaaacttttaacatttcagatactttgtcccaggtatttcatgataaggattgggctatgactctgttacagaagggccaaatgactaaaatgtctctgaacaatattgattgcaaatattctacccagttgtcaggtcaatatgttccaattcggaatttataacattgtatctctactcccaaaccatccaatctcacctacctcacttccatattatggtgggtgatctcagattatatttaagctcatggttacttgtcaagtagatatggagtttagcctaacttttgaaatttatgctgagattacccttctcattatagaattaagtaggcagtttccaagtttagatttagcaggcagtttttttcaaatcacttaaaagttatatttttttagggcattgaacaggtttgaaatcctaccaagatgtcatgtacacatagaccaatagaacagaatagagaacacataaataaaactgcacagctacagccaactgttcgtcgacaaagtcaacaaaaaaataagcattgggaaatggattaaagatttaaatgtaagacttcaagctataagaatcctagaataaaatctgggaaataccattctggacattggcttgggaaagaatttttgactaagtccttaaaagcaattgcaaaaaaaaaaaaaaaaaaaaaatgacaagcaaggacttactaaaataaagagcttctgcatggcaaaataaatgatcaacagagtaaacagacaaacaccaaatgggagaaaacttttgcaagttatgcatctgacggtggtgtaatatccagaatctatgaggaacctaaacaattgaacaaacaaaaatcataaaacatcatttaaaaaatgggcaaaagacatgaacagacatttctcaaaagaagatatacacgcagccaataaacatgaaaaatgcgtcacatcactcatcatcagagaaatgcaaatcaaaaccgcaaggagataccatctcacacccgtcagactggctttgttaaaaagtcaaaagacacccaatgctggcaaggccgcagagacaaggggatgcttatacactgttgttgggaatgttaattagttcagccactgtagaaagcagtttggacatttctcaaagaacttaaaatagaactatcatttgacccatcaatcccattactgagtagatatccaaaagaaaacaaatggttctaccaaaaagacacatgcactcacatgtttgtcacagcactatgcacaatagcaaagtaatgggatcaacataggtgtccgtcaacgttggattggataaagtaaatgttgtacacatacaccataaaatactatacagccacgaaaagaagaaaatcatatcctttgcagcaacatagatgcagctagaggccattatcctaagcaaattaacataagaacagaaaaccaaatactatatgtactcagttatgagttggagctaaatgttaggtacttatagaattgaagatggcaacagtagaaactagggactaatagaaggggaaaggaaagggggagacaagggttgaaaagctgcctattgtgtactatgcttactacctggttaatgggatcatttgtatcccaaacctcagcatcacgccatatatccaggtaacaaacctgaacatgtaccctctggatcttaaaagttgaaaaaaaaagatgtcatataaatattcgtggtcactaaaagtatctaatgtattatacataaaaataaaaattgggtgaattggaagtgtattctttgtatcaagtcatgtcggagatcctattctgctttgatcacagtgtgaattcttttgcatttttgttaccagtcacttctttatttattgaactaataattacatattctgataatctgtcagaaagataaaaacattctttgtccatgtgtctgaaaatttttaacctatttttctaatgttttaagtgagaagagcatgttaatactgaaattgtaagcagtagactgaaaaatcatcccaatccatgggttatatattgaattgcttttaactgtattactaaatattaagcttaatttattttatttctacatatccccatttccactataggtgatttgtatgaatttaggaacttccttctctcatccatttttatattaaaactcagactttctaaaacaatatttctatccatccatcgttggtaactatgtactgacatgttttgtgcatccgaaaaatgttagcattagtttgtgcgcacagaagtaattccagtcaccatatgatgagctgatttatttatttcgtaagtgtgttcattattattatctcttcagcacccaaatatataggggacttaatgatacctacaagtaaaaacggaagacaaaaacgccctgctctctacagaggttaaaatgtttttgcaacagggctctagatctcagctgtgaaagtagggacgagatgaggctaggcatgcagtgtcagtataatacaatataatcaacatgtcagcatctaatgcaggtgttgcaaaacaaaatgtacacatgggtagtcaggtaacagaaaagcatgaagtagtaagggctatctatgcaagaggttccaagctgactatatactgaaatatttaaacactatgtggggcaaataaaatggacattagaacagttcgatggtcagttggggacttctgctctttcttccagtctctgaacatatcttaaagccacaatcatctatttttatttattgttatacatttatttataagccagcacccctgtgatttaagttctgttgaaatgctgagttggaaaagatcgatggatgggggaaatttagtgcagaggttttgccccaggttcaaaatcctttataaaatattaatacatggaacaaatattgaacaattaaaccactgataagttaatcaatctgattcaaagtacacctgtgaagagggacatggcaagaaaaatattacagtaagaactagaaacattccttcatggctgcttgatatggatatgtcatgtttaagaaaattcttctttagactgttgagattttttttcctgacaaagaagattcactgtcgaggaaagaaagaggtactgtgaaatttgttattgaaaacatgcacatacttttgtcagaatgagttaaagagtgaacaaaatgtgcctattacttacgtgttgtgctgttttaattcaagattaaaatatttaacgtccacagacaagaccacttttatatgaatattatttttctgctttattgctcaattttattaccatttcaaaacacccgtgttgctttctatggccaaagatgtttagcacttttcatggttatacttctgtacagtccaaaatacaacacttactttacacatacacaaacatccaatgtattttgttttctgtcaagtaaagacaatgtctgtgttattaagttaaatgtcactttcaaatacaggatatgttgatattagaatgttcaactttatttcctcatttaagcaaattacagtgtgaagaatgtaactgcagcaatttataaaaatcatatcacattcaattatgagagcaaacttgttttgtagacttgaactagtttcaattaatcttggagttatcatttcaaaaattctaaacagagagaaatacggagtgtaataatggtaggtctttgggtaagctgcttccaggaaaagaaagcaattatatatgttcacatagcactgacaaggagaaacaaaactttggacggcaaagaacttgcattagtctttttgacatgttcctgtggtgtgatttattacgtagacaatcagctcaacttctcaagtttgatatccttggaatcatttgaaatttaaattttaatgaaaattcattaattccaaggccaaaagaagtgattctaattgcttttgagaatcagactatgaaagaattctttggcaaacttgcactgtcttttctcttttatcattggttgcttcgtaggtacttaattgaaggtcctctgattatcagcacgggctgacatcagttcactccatgcattttaaacagtaggccagatgtttaaaggatcagctgaagcatcgatagcatgctagggtgaataataaaattttcattatctacaagaagcaaataaaaagcataagcattttcccccattatcctgaaggagaagatgaatgcctaagcaacattttaagaatgggttgagtgtggcctgtgggaaaatttgggtagaaaacttgtagttagctaatgtatatactgtttgcctctttagctcaccatatacccacacacatgggcatgcatgcatacagacagacacatacaatacacacaacaaacaggaaattcagatatactgaagaaatgtatttaagggattactaagtttttgtaaataaaatcctttaagatgctgagaaacaatggaagagaagtaggacatgatggctcatactttcgtaatttacttgtttaacgtttgccaaggtttaaattaatgtagatgtttttgtggctaggattaatgatctaacagtttggaataattaggcacttttatcacctagaaagcccagaaacccagcatgcaaaaattctggtatgtctgcattttacacttagatataacagagaaatgacaagtagtcaagtggatagagaaacgaatgattcttcacacatgcacacacacatagaaattgtctttttaatagtattttaatgtaacacatttatgcataatttctccatagtgtttatcttatagtgaatatgtgatgaatagtctctaacattagtggttttatagattaaacataattaaggctttatatattaaagagtcaattggtgacattctaatataaacatgtttatctcatatacattgaaatattagataattcattcgttgagaataaatcgaatgagtcaaaacttttaacctccactttgagctttgtaatagtatccactgaaaatattcatgaaaatttttaagtcatttctatttatatattcagtccaaacatctcacaagtttaaaatgtaaactcaagaatataatttctgtattctacaattggaagcatccatcatatcagatgaacttatatagtttgtgaaattttgcaaactttctgtttagtaaatcttaatgtcaaacattttaacttccaggttgtctttcttttcagttttaatatccgcgatctttgtatactcgttgaatggattctcaataagtaacccacaaatatatatacatactatgtacctacaaaaaataataaaaagtaaagaaatcgacacttatccatacctgtcccatagtaataaactattcataagtatatttgaaagatatgagaatcataaaagttcgtgtttgcacccttttgtgcgtggaatcctaggtttgcattttgtggatctagactttttggagtgtggaaataaatgaaacaaataatcgagacccagtcttatattcaggttatcattttactacataaagcataaataacatttgcagtttgtttctatggctagctctaaagtcttagcaacgagaacattatagaaagacttcaactgtagcttccagcagaacttctgaggttccgtttatggactaagcagcagttgagggggacaaaactcataggcaattgatcactccaaaggatagattgtcttttctaacctaatcaaaagatttatagtgaaggcatattcagattttgttgaaggatatggatatataatcatgtgtgtgtgtgtgtgtgtgtgtgtgttagacatacttaaaacattatttgagtagaaaattctgcacaaatggaaaagtataacatgtgttatatccacacatgttgagcatttacctggctgaaacatcaaaagctgaattgacttaattgaatgttgaatacttaatagttactttgtagtgactcactattaaaacattatctcaagctttgtcagaattaatttttttaaaaaactcagattagtgtcaggtttactgaaacagcagatctgaaattactgtgtttttttttcctttcaataatcagtttctaatccaaaattgaatatcagttccaactctacattcagtttctgttttacttgtttggactggcttttggttctgttttccacatagatcctctctgtgtaagacaaagccatttgtgcagattaaattttactgagcgtgttaacctatttaaaacattcatccaaaaagactagtatgaattcttcatatggcaagctgcttgttttaaaacttccatttattctaaaatcctttttacttatactttttaagaaacgtattcccgatatacaaaagtaacacatgctcattaaaacaaattaaaaatagtattgtataaagagctgatacatttctgccttgccccatttaactttcttaagtgttcatgtgaatcatccattcacatcaagacatttatctgtattcatatgaacgtgttttaatatatataacatatatagaattttatataaactttccttttaaaatagaaatgaaattatatgatatatttattctgtgtctagctcttgtcacgtaattattcaagaacatatttctaggttaatatctgtattcttaggtagcattcactaactcctcatctacttgttttcttccattctaattgtgtttaacatttcttcatacaattggttgtcatttggtcttctttcatggagggtgcataatgttcattctcaccaattctttacactttacataactgcttgatacgaagccagaccttataaatatcaacaaagcaggaacactgtaatcagctatcagtttcagttgagctgaatgaccctgaatatgtgtacacatattttccaggagattttaaaactgacacctcagatttctaagacctggagaaatcagcatgagaaacattgatctatattattccgtgaaatgatttcactaaatagtgaagcatctcccacatgtggactctgtaatttattagaataaagagttcatgtgcttctgaagaacttgaactactcttctggcctccgtacattggtttcttagctataggaaggctgagcatgtttttcctatgcgtttcctttctagctcatcattttagtgacaaaacaatctttcgtggtgttgctctagctatagaattgtttcagattcatttgaccaaaggtggcaaatacaacagtcccaacaaaaacaaaagacctattacagaatgatggaaatgaccccagggaacaatggcacctccacatttcttaattccaaggttataagcagtggtgtggacaattctcaattccaatgctgaatcgccttctaatttcaaatacctgtgctaaaaattatttacgtctactgaaataatgaactggaccccaccaggaatggccgatatgcttgtagtcagagcacaactgtagaaagaaaataacattttaatttatagaggtatgatgatagctgtttcatactgttttcagaacgatgaatggcctgctcagtagtttcttgtcatcgtactgagacactttaatttcttaccagctgagatgaggaatacgagcccagtgtgcaggtgaaattggttaacaggagccattaaaatttggaagagtcagaatagcatcaatcaaaatgctttcagtgtaggaagtaaacatgtactagcctgacccacctgtcttttcttttaggtatgttggtaatattacaatcattttgaggtatccataaacaactgcttagatctgaagaattgtatatctttctttactctgccctggcctggggttatggttctcattgagctctaacctttcagaaaaaaaatgtagagaagtggttcaagaagaatgctttatcttgcttcataaaaatgatagtgatagttttattgaaggcttactatgtgccaggccaaagtgcgttttattatcgttcccattttccaggcaaagaagctggagcacagagaggctaagtgagttgtccaggatggctcagctaacatgctgcagttgggatttgcacccagaccaacttcttttcaaccactgtcccatcctgtgtcttctctactcaaaaagtgtttcagctccaaacctgaaactttaaagaaaaggaaatccttagtggaaagactaggttttagtcacaaattatctccttccttacattatttgtctctttttcaaatactccaagctttgattaaaactgtctatcactaggaacattgtagaattgctaaggtggaattgttaaaagaactcaattccaattaactttgccattgattactgtgtgttctggaggggtgttctttctttcaggttaatgatgctttattgtatatctcaaagattaaaaataacaatgaaggaagtagcaaaccggaacttctctcacaatgcatctttcaatctcgtgctttaaatgaagataaaatcatggctgtggtaaggttgcaggaaggatgatatagattaagtttcttgcaaactgccctctgaattttcaatagctgtagaaggtattggttttccaaaaaattgacaaattgaggattcattcagcagtttttttctaggtctcttaccagaaagtgatcactaaaaagtgtagggaaaccactcaaagttggatagatcattattttcacttaagcattttaatttcttgaaggagctttataatgcaacaaagaatttacagtcctgtgtcaccgcttaaattttctagggtcatcagtaaactcagtggaaataaattagttcatgaatataattgacccttaaattctgtcactgtgcaagtaatcggtgggtctgctggatatggctttcgagcagacaggtcaacttcttcaaacagagaagaagcatagcataaattgaagacaaataacaaactacttgtttcctccttctttggcatcaccctatggatggagtatgcatttataatttaacacaatcaagagatctttattatcctacttttgggtacaactgcttcgtttctcttttgaatctctacagctatttaaaaatctgttttgtaaaattctttaaaaaactaaaacatcagattcatatttcaggtatcttactatcttataccaacttaagcatccagtattatcacccacccttcccctgagtgaatccttagcactgggctcttcctgttttatccctgtgcatgctgagctctttctggccttcaagtctacttccgttgcaactgttgtctgaatggtctctctatgtccttcttactctctaaatatttcggaatttaaagcctggaataatctaccttagtccaaaagatatgctacactattctagttcacaatgatctcacactgccgttgatacacaacatttaatatcaacttaatatctatttcagttcattacgaggtcacttatgctacatcttatattgttgccttggacttttattatctcttcatatatgtgtttatggtgctcccaccctcacgagaagttgcaaataccatgttagctgtctgatggctttctatgttgtcaggtataccatttcccaaccagttggcattcaatgattaagttcattaacaaagaattgtatgtgttgaaaaagatgtttttttcttaatgaagcacttgtttttatttttttaatgaaatccaccctcttaataaattttaagtgcacaatacagtattgttaaatataagcaaaatgttgcatagcagatctttataatttttttaaccctacatgcctgatagtctatacccattgcacagcatctcaccatttcttccctcctccagcccttagcaaccaccattgtactttctgtttctataattttgactactttagatacctcatgtaagtggatgcgtgcagtatttgtccttttacgacttgcttattttatttagcaaaatggctacaagattcatccacattgtagcatatggtaagatttcctttttgtggcagaatgatattccattgtatgtatataacatagctttatacattcccctgtcaatagacatttagtttgttcacacctcttggctactgtaaaaatgctacaataaacatgggaatgcagatatctcttcaagatcctaaattgaattcgtttagataaatatccagatgcgggattgctagatcttatggtagttatattttttatttttttgaggaaactccatattgttttccacaaaagctgcacaattttatatttccaccagcagtctacatctccaattttcctacaccttcaccaacacatgtaatgatcttgggctttttttttttttttttttaataatggttatcctaatccgtgaggtagtatatcattgtggatttgatttgcatttccctggtagttagtgatgttgaacatcttttcatataactgttggtcattttaatgtcttctttggagaaatatctattcaattcctttgttcactttaaaaattgggttgttcgaatttttgttgttgttgttattacgttcctcatgtattttagatattgacaccttatcagatatatggtttgcaaaccttttctctcattctataggttgcttttaattctgttgattgtttcccttgctttgtagaagctttttagtttgatatatttctgcttatctagttttgtttttgttggctgtccttttagcgtcatatccaaaaaaaattattgtgaagaccaatgtcaggaaatttttcccttatgttttcttctatgagtttcatagtttcagatcttatttttaagtctttactccatttcattttgagttgatttttatgtatagtttaagttaaaggtccaattccattctttgcaatgtgtatatccagttttcccagcaccattggttgaagaggatatcctttcccagttgtgtattcttggcacccctattgaaggtgatgctaggtttatttctgggatctctattctgttccattggtctatatgtctgcctttatgacactatcgtgcgctcttgactgaggtagctttggtaattcattttgaaactagcaagtgtgatgcctccagtttattcttcttcctcaagactgttttggctatttggagtcgtttgtggtttcatatgaattttaggaaatttaccttatttctgtaaaaaatgcgattgggattatgataggaattacactgtatctgtagatggtttggatatatagacttttaaatgacacatcagatgtatttccatttatttttgtcatcttcaatttctttcaacaatatttcatagctttcagcacacacatcttttaccttcttggttgggtatttactaagttatttattctttttattgctattgtaaatgagattgttttctaaatttcctgtttttatgttgctagcgtatagaaacgcaactgttgaatgatgactttgtatcctgcaactttgctgaatttgtttattggttctaaccatgtctctgtgtggcgtcactcttaagattttctacgtatcagatcatctaatttgcaaacagatataattttacatcttcctttccaaatttgatgtattttatttctctttcttatctaattgttctggctagtacttctggtacgattttgaaaagaagtggcaaaagtgtgcattcttgtcttgtttctgatcttaagggaaaagattttcagtcttttgccattaaatgtgatattcactgtgggtttttcatatacggtttttattatgttgcggtaatttcgttctattcctagtttgttgtgtgtttttatcatgaaagtgttgaaacttgttaagcgctttttctgcagctattgagatgaccatagatttttagcctttgttctgttaatgttgtgtatcacactgattagttttcataaattgaaccatttttgcattccaagaataaatcctatatggctctcgtgtataatcctttcaatatactgttgagttcagtttgctagtattttaatgagttattttgcttctatatttatcagcggtattgttctgtacttttctcctagtgtcttttattgactttgatatcaggatactgatgccccttgtagaatgagcttggaagtgttctcttctctttaatttttctgaagaatttgagaaggattggtgttaattcttctttaactgttcattagatttcaccagtgatgacatttggtcctgggcttttctttgttggaaggttttggactactgattcaatctccttactagtttcggcctactcagattttctatttcttcaagattcaatattggtagattgcatgtttcaaggaatttgttcatttttttctaggttaacatacagttgtttacagcagtgtcttataatcatttgcattctttttggataccagttgtaatgtctcctctttcatttctgattttacttatttgaattttcctttttttttttttttttttacttaatctacctaaagatttgtcaattttattgatttgtttttaaaaaaactcttagctttgttgatttttctattgttttctatttcaattttggcttttttctgatctaatcttaatatttccttccctctgctaactttgggcttagtttgtccttctttttctaagtctttgaggaagaaaatggcaaggacatgactttctttagcagttggaaggacaatgctgtaaatactcaaaaattaattatttttatagtgacaaaaacaaaataaaaaacacttcaaagcaaatgaaagtttatcatttaatttatcaaatcactaagcagactgcttgatcagagagaagatactcatatgatcacataaaactgaaagattaagaggtaaggacattcatgttatcattacatctaactttcttatttccaagatggagaaactgagggttggagaaaaagaaagatttctttgttagatacaaacagacaggactaaactcagtatagcagcctcctaaattccaaagtatcatgatactgtgattttatgcattcttcagaaaaatagtagagccactggattctggcaaagaagttatataaaatgtcaagttcttcctttgcctcagaaatgaagttttatgttccaaaattgattgggaagttctccttatacctcacatcacgtctactattttacattgtttacttttgaagaatttttttaattgacaaataataattgtacatattcatggagaacctagtgatgtttttatatatgtaatgtatagtgatcagatcagggtaattagcatatccattatctcaaacattggtcatttatttgtgttgggaacattcaacgttctccttctagccatttgaaacttctatattattgctaactatagtcaccattcagtcgtatagagcactagaacttatttctcctatctagctataatttatttttaaatatgctttttgaatctgttactataaattgaatgtcacatcgttttgaaaatattcttaatttatgctcaacaggcaagattacacacctgtgataatatctttaatttaaaacattactctgtttaatttaccagaatatggaaccctagtcattttagaggtggagcaaatttcagtgataatctagtgcaaatttctcatcttatgaatgaggagattgagtctgatataagggacgagattttcgtcaatgagcagcttgttaacattagctctgtgatagaacacaggcacttgtcctcccaggccggtgtttcttctactctatgatgggctgttttgttgtagtttttaaacagcagcattttcaccatgcatagttttcttccaaagttcgttcttaacgtttttgcacagaataactagattttggaagtagaaaaaggaaattctctttgcatccttgtatctctggttattttctttgtcctttgatctctctctcctcccctcccctcccctcccctccccttcccttccctcccctctccttcccttcccttcccttccctcccctctctcacacattagagaaagagttaaggtattaaagaatacataatactattaaatttccttcacatagagaaaggaatgaaaaaaagtgaaaaatggtcctcaccaaatgtccaaacttctgtaggtcatttccatagtatcagcaatgtcctgtatggtgcctcggggatatgtaagcaaatgagcaagtggttagctaattctagctttggcaaacacttgttatggcttacttgaggagaagtcacttctccaaagtgaaaataatgtgcacaggtcaattagaatttttttgtagaaaaggaaaatactttgtagggacatggatgaatctggaaaccatcgttctcagcaaactattgcaaggacaaaaaaccaaacaccgcatgttctcactcataggtgggaattgaacaatgagaacacatggacacaggaaggggaacatcacacaccggggcctgttgtggggtggggggagggtggagggatagcattaggagatatacttaatgctaaatgaccagttaatgggtgcaggacaccaacatggcacatgtatacatatgtaacaaacctgcacgttgtgcacatgtaccctaaaacttaaagtataataaaaaaaaaaaagaagaaaatacctccttatgctcctgacttattttctttttggttcctcagtcctcttctctctctctctctctctctctctctctctctctctctctctctctcacacacacacacacacataccccacatatacaatatgattaaggatatatgtgaataatgaaagcttcttgtgtatagatttagaagtctaatggacaaaatcaatattttcctatgtgcatttaattcccccctttgatttaggtatatagtctttttttaaaaaagagaaaaaaaattaggtgaccttaaggtatagatcctactttcaaaaggtttacagaactagggagaggaacatggacaagatttaaagaactattttaagcagaataaaatgtgatttatgaacaaagcatatattatttgtgcgtatgtgtgtgtgccaacaaagatgcaattaggagattgcacagggagatgtcattagaaccaaccttaacgggtgagaagtctttgaagacatttagaacatggaagatctctgacagagggaacaaaggcatagtgacaaaagtcaagggcatatttaggactggagagtggtatgtgtggcttgagagtgggcgagaaaaaacaacaatgcctctgtaataggaaagtagacagaggcatgacattaagagctttgccagctgtgctaaaagtagtgaacaagagctaacaaagtgaagaaatgtaccttttctgatgtgtatcattcccttattcatatacttcttgagggggaaattcattctgtgttgatctagtaaactactacaggaccaaatgataaaaagaagtataggaaagaatgtttcagcatactttacgagataacttccttgtagctattctccatagtattttgagcatcacaaagcaatgagctgaaactgtctaagccaaaattgacttgtcatctgttagggatgcttagatgagaattctacatttgagagcttcttagattcattgaccactatgtcccattctaagatccatgaatgcgtgacctaactattacaccttcttttagtctgattgtcaattttgtattttcaattgtgcaagtttctaaaactattttaggaagataaatctagcagtggtgtgggaatagacaagagagaaggggaaagactcttcaggaaactaaactcacaatttatgagtattctttattgcccaagtcttcccaaagtctttcatcaagaaagaggcattgcaactctccttttatagtttgtttttattctggagcagtgatgttttggtggagttgttcctcagtgcgtaattaaagggcctatgacaattacagttcatctcctgctgctcaaggtactgcagatatttggatctactactctcattcatttccaattaatgtcagctttagatttccttcagtatgctatgttataaaatttgattatcgttgtgcccaccttcccacttaatttcaagcaggtttctcgattacctgactaaactaatgaaatctgactaacccaatatctgtggacagtagtgtgatgttactgatttttgtatgattagtcaagtcatattcatgccacgttttcatatagtaccataaaggatattcttctcgtggtccttttcttttattctgaacatacaatgagaagaccggtaaagtgggctaggaaattaaagaaaaatacaaatggcaaaaaatatgggtcactcgaagtctagaatagagagcacaatcaattttgaattaaggggtgataaggtgatttggtcaggtgactggtgaaacaggaaagaaactatactttttgaagtgtttcatccatgtgttaagattcatttggggtcaagaatctaaatttcatatccctgggagtggaaactaagtaaaaaaaaaaattatggaccttggtttaatagctagaggagcaagagtgtatctttatgtgacttaacttctatgtgaaaagtgaaccttaagattaattattgggggaatttacttactcaggttctatgcctagatggtctgcccaactaagaaaacttattttcctgttactccatcctatttttcatacttttatactgcacttgcagaaaagcatatatttctacccaatacgaaaattcctgggaacatatttttctacatttcccaaattacttcaaaaagtaaacttaggttatttcatgatctccattacaatggacaggtggccttattgaatgttgtcctgtgaatacaaagatccagagtttaaagaacaaggtgtacttgcatctcccacttagggtttgcttgtggtggagagagaatctagtttgcttaaaaggatgacagtgcagtgccccaaaatatctgatatcattaaaagtctcatatttgtctttcgtaacttctctagggctgtcgatgacaggagacccttaactcctatgccttgattatgtgaataagcacatgaaaatattttagttatcttagttcacttttaaactaagtttcaattatcactagattctaaatatcatcattgagccgttcttaaggaactgattttctacatattcattcacttcacctatatctagtgtgtctactatttgccaagaaaaatttactctcttaattcagcattccatatacttaacatcataaaaagtaggccatttttagttttctaaattatttatttaaacatttctttaaaattacattctatcattacactatatttcaacactacagtaagcagcctattttgtgatttttccttatataaaatacataattgaaattaaaaatgaagttaccaagagccattttcactctggggaatgcacatttataaattatggggttattttttcttcatcagctttcatattattaaactttgtctcttcataattacagagatgactagacacagaagggaatttaacatttggtgtgcatttgtctaacctatactttatgttagaaaatacatttccatttgaaaaaaaatcagtaattgtgggtgtgatcaagagggcagcctgaaagtcgggtgatgtgactcacacctgtaatcccagcatttttggaggccaaggtgggattatcgattgagcccaggagttcaaaaccagcctgggcaacacagtgagagcctgtctctattagggggaaaaaaaaaaaaagaggaagttagcctgaggcaatgtaaatgaaatacatatttcaaggatatttatacatgattcacgttattcatataaagatgtgccagagaagactataggtacgttattttacactattttgctaggattttaagaaattcaatgtgtttttatttcagttaacttagaaaacttacctaacttatacttctcatggacacaaaagtttttaaagataggatcaaaaagcccacatggtgaagcattttgaactggatgaaaaacatctattatctttaaaattttatgatattactgattgtaatagactccctttttaagaaatcattccttatagaacataaggtttacatttacaatcaacaatttctatccttactacaataaaggcacatataaaaagtacagttgcatatttagcaggtttaattgtacattttaatgtagaaatcaattcaattctttcatttatcagcattattacagtgatttcaaattaagcataggtaactttgatatagataaatgatgtacacagcagttaaattttattttcaattatgtagtaattgtataacctaggcagtataatttgtaaactttgtattttattattatgcttctcccacttggcataagcacaacacttcctaaaagcataattttctatagacttaataactccctaaaaacctgttttggacccctatactatttgatataggcagaaaaaaaacataatccatgctcaaatttgaaaaatgactggtcacatttggtataatactaaaggtaaataaaatcaagagtctatgaacatttccggacctgcacatttgttttattaaaatgcataattgtctttagtgtgtttctatttgtttatactctactgattttaattaaaaataccaaaatacgtttattaaaaaactgtcagaatctaagttgttaaatatacttaactaggaaagtaactgtttaaacgagataatttatagagaaatgtggtgtattgccaattagatgtcaagatacaatacaactgataatgaaaaagtagcattttcttagggatggaatacagtgtaaggaacaccccagtaagaatacaaaaattactgaaaaaaaatcttccttcctgaaaaaccaagtgcccttcaagtgcagaacctcatccaactaattgttaggtatcactaaagcctgataccttcaattttctggatcattcaagctgtatttttgagtccttatactagaggaggtaaagagctataaaaacacttaatggtatctgatgtgaactgtggatcactttgacccatcacttctacgtctacatcttggataaattcccattgttgtcatagattgtacaggtttaatggtgcgtttgtggagggggctcgcttatagaaaatggagactctgaagggataaggaataaatgtatcacttcaggtcttttatttgaaattggggtccagagagcctttttgtatcagacttgtcaaaccatttccatttagtaattatatatgcactagcacttattcctacttacctcacctctttatgcccatttccttgtagttgcggttatgcatgaataatttattgcaccccttaccaacaatggaataaaacttccattctgaaagctttccatactcatttccaatagcaatagggtttttttaacggacgtattacaaatgtacgagtcagttgaacatagtattcctctttgtaagaactccaagtggatgcatgctgttgtctcaaatctcaattagaccttgctttgaggtcccttcattgccagtcatctgttctccttcccctgacttgagtatttctccagatatagataatacattttcccaactctgtgttccaagaactgacagtggctttcattcattttgtttgtttgtttgtttcttctcgttctcaagtatcccgcagtctactgtttcttccctccattcgtttgtcctttcagagtttcaaaatccagcataggtacttcttctaaaatgtcttacccttcacatacacacaccacttgagaccccatcagcctctgtccacacagtttggttacattcatagactatttttatacatcaaaatatttgaaaattttagggtaaatctcagtagtcattcatttttgctcttattcaaccaatactagtcaatcagcctgtgccaggttttgttgcaggtaccaggtatccatccataaagaaaacaacgtccctttgttgtggaatttacattttagcaggggaggcaaagaacccaataaatatgataaaatatcagattaaaagtacgatgaaaaaaatcatcagggtaaaggaaaaagggaagcagtattttagcaagagtggtgaagagaggaggctgagagtgtgacatctgagcagagacctaaatcaagtcaaggaatgaaacatgctactatctaaagaaatgagtcaggataaggaactagtaagagccgaggcccagagatgtgaatatgctgttccaggaacagcaaagagactggttgatatgatgtgaaaaatgagaagaaaccttatgatatgtgtcaagagaaaaaaaaaatttaaaagcatgcttgggaacggaggcctccagatgaaaaaaaaaaacacagttcaaatccttgttcatgcatttagtttgctttgcaatcttgggcaaaatgttaaatttctgtacgttttatcttcctcatttttaaaataggcacaaggacatctacttaataggttcattgtgaggagtaaatgagatgatatatctaggatgcctggcattatatcatacacttaataatacactgaataaataatagttatgtctatttatttccttatcgtttttattattatttcaatgcacagacctgttcataagataatgataaatattagtggcagaaactgaagatgttataaattattaggaggcgggaccactcagttcaatgtatctgttttaatatagtcagcaaaagtgtgaagataccaacaattaaatttcaatgcattcttccatttcactagttttataaactgatgaactaccagaatgtcaatgtatgaattgcatactcattcttaacaaacagatttgcaaaattatgtgtaaaattagccctcagccttccaatttgttattgtcatatttcatggaaatacataatctgtaaatttttgttttaatgatatgtgaaactgcctaaagtagagtcttggcaactacttcacatttgtcctccagagatagtggataaaagtgtcaataaatgaacactctatattcactaatcacaggcaagggacaaggaacagagtggtcacaaaataccacaaaattaaagcacattccaaattaaatatatatgtttttattacagataatgtttgctagactctttctaattatctgcaaagattttaggaatgttttaatgttttaatatttacacacctgtgtatttcaagttcagtcaaacactattgttaaaactaaatcttctcatctctaataataagatgtgaacttatcttggaaggtggttattaggatgggagagataatgtatttcattcaaagtaaaaatatttctctgtttctatctttctctttctctgtcatctatttatcatctatatccaggtatctatgcacctatgtagactagcattcaatgaaccatagatattattagtagtagaattgttactaatattaaaataagaagtatttaagaagaaacatgtcctaaagcataaggtcaattattactctcatgttttttggcatatgaagcctaaaaagtgtcaatttcaagagagtattaataaagattgtgataactgaaaggttcctgcttgaaattttgtgtggtcttacaaatatataaactctaagcatttcagtgagccaattactgactaggcactatgtcttatgactcttttgtcatagtatgtaaaaaacaaagagtagagacatcataaaaattatagtagatgggcactagggaattacgcaaaataatttgtagatttaatgtgaaaccaaaacatctgttcaagtcaatttcccacaggtcatgtggcaaagagtatgagttccagactgaggagaggaaaaggttgttcttccacagggaaataaactgagtgtaataaacataatttttcttcttaagcattatttaaaacaaaaaaaatgccattaaatctatctttcctgcctctcttatcaatgctcccttccctttcaccacttgtttcaaactccaagccttgggattttattttggctttttgccttaatgtaactaaaatgagagcatcacaaatatgaagctcatcaaataatttagcagcattttcccctgtttttaactttctctttggaaacgtagatttcgaaatttaagggcccaaaatatgaaatgcaattataataggccatttgttcattcagcttgataaacttgaataaatagtattgaacttttaatgcaaaaagaacaaaacaaaatagaactctccacgaagaaacttttcaatgtttgcatttctgtgtgaggagaagggtaatgaatgtgggaaccttaatggaatccatgttcttccagtgatgacaagggtcaaaatggagaaaaatggtcactttctacccagtacattatattagttctatgtggacaactataacatagctgatgctggttttcaggccataaatgtaggtatgtattttcctactatttataaggcaaaatttctatttgtttaatgatttctatataggtagattattctgtctttaggattaaaaacgacctgtagaccaagagactttctaatgtccaccttagagtatatggcttttactgttacagtttccatttcctttgcttgcccctttgagagaaggaaaggagacatttgggatacatacatcaatgaggagctattaatgaataaatgaatgaaattgtcagtcaatttatccacatgatcatcaattgccaataattttatcacctctgtgggattaagtagaggtaacagtttagaaatttgattttttgaaagcatttaaaatgttcaaatatatcactctggtaactaagggaaagtgtattattttcttatgcttagtcttattttggttttgcctttttaatttaaattgaacacttatatcaaagagcttgcaggattataatttgaatttttgaagcaaagatcattttcttaacatcaaacaaagagtagatacaataggaataaaatcggcagaaaaacaagagtatcaaggacagacggggagggtgggtctgtgttagcatgtattgctatgaagaaatagccgagactgggtaatgtatttttaaaaagagctttaatcgattcatgattctgcaggttgtacaggaagcaggacaccagcatctactcagcttctggggaggcctccgggagcttttactcatagtggaagatgaaacaggagtaagcatgtcacatggccagagcagaagccagggggaggttgccacacatttaaaaaaaaaaaaaacaaaacagatcgctcaagaactcagctgctatcatgaggacagcatcaagctgtgagggatccacctccgtgactcaaacatctcacaccaggccccaagtccaacacttggcattatatttcaacaagaaaaaaagtttaattggctgatggttctgcaggctgtacaggaagtgtggcacaggcatttgcttggctcctggggaggcctcagggagtttttgctcatggcagaaggtgatgcccacacactttaaaaaaaaaccagatctcatgaaaactcactcactacactgaggacaatacaaaaccatgagggatctgtccccatgacccaaaaacctcccgccaggccccaccaccaacattgggaattatatttccacttgagatttgagtggcggcaaatatccaaactatatcagggctcatgtccagttatatgtcaacatgcctgcattcgaaacatcctgtccaaatcactgccttgtcataatacttatatttttctttattgaatacgaacacaagaagattaaataatagcatttctactttaaaacagtgggcaccatattaacattggaataatagtagtaataacgatagtaataacaatgatataggctgggtgcggaggctcacgcctgtaatcccagcactttgggaggccaaggcgggcggatcatgatgtcaggagatcgagaccatcctggctaacacagtgaaaccccgtctctactaaaaatacaaaaaaattagctgggcatggtggcaggcacctgtagtctcagctacttgggaggctgaggcaggagaatggcgtgaacctgggatgcagagcttgcagtgagccgagatcgtgccactgcactccaacctgggcgacagagcgagacttcatctcaaaaaaaaaattaaataaataaataaataaataataacgataaaaggatatgtgtaggttttttttttaataggctgttaacattaataggcattgtgatttcagggatatcatcaaacatcctggtcctaagacatcccctattgaataggaagggcttaagttaaacttctcatgagccacaattttctgattatatgtttggtgtgtgtaatagccacctcagtgatgatttgattagcctggacccttacataatcattgaagtatacccatgttcctttatatacttctttagtgttgaaagctcaaaattaagcaaaatagtccccttgataatgtttagattcttaacatttgctttctaaagctggcaaatactctcttcccagtgtcatgaagttaaataacatgttgcttagtgaggactttaatgttgccatgccataggaagaccttattcgaaatccccttacctgggagaatgtcagattattaccccccaacttgtttaacacttttaggattttaaaggtgttcacatttgtattagaacaaaatactattgagaaacatttctagaaaaaaattatctttccaaattaaaatcagtggtatgtaatgtaggagtctgattataatgattaaaatacatgggctttgggcatactgcctaggtgaaactcctggtttattgcatcactattagtataacctatgggagttaacctacgtaagcctcagttaatttttctctcaaattgatctaataatcgtctctcataggcttgttttgatagatatttcagtgtatataatatacttaggacagtgcctgatatcagtaagtctccttatatgctatttttctttctattttaattatttatgcaagagaaactattatgctttaactcaattaaaataaaatgcctttgtatttattcatgtcaaaggaaatatgcaagtattgcattcacttcctaggtgcctttttgaattgagctttgcatggttagtttgtataaaaggttcagtgaactttctcataatgattttttattgaacatatggaatccattaagtgttagcaaaagtcactatccactgagctgtgtccaggggctgacagttatgtctatctcttgcaaaaataaacacatacataaatgcactaagacgtatattacctgtcgtcatctcttagagcatttccatttttcttttaagttttttctttcaatgggttttttatctttgtgagtacatggtaggtgtatatgtcaacggggtacatgaggaaggtgtatatattgatggggtacaagagaggttttaacacaagcattcaatatgaaatagtcacatcatggagaatgggttatctatcccttcaagcatttgtgctttgtattacaaacattctaattatactctgttagttattttaaaatgtaccattaagttattactgactatagcaaccctattgtgctatgaaacagtagatcttattcttatttttctaacatcttagaacatttccacaaacactacctgcttgttaaatatacctattctaatcttcatataatcaattacttttttcctctagaatgtactatgacacatccatggggaaaatgtagtaatctaattaagactatttcctctcattttatatttaaaagaatgtgctctatcaatttatttacttgtacagccgtaggcaacctctaaaatatttaaagttcttaaaagtcagatatttcagttaatattgtgattatatagttgattttgatgaacatgttcatctaccagaaataaattatacacacacattgatatggttaggctttctgtccccactcaaatctcattttgaattataatccccgtgtgtcaagggagagaccaggtggaggcaattggatcttgagggtggttttgcccatgctgttctcctgatagtgaatcatgagatcagatggttttataaagggctcttcccccttccctcctcactcattctccttcttgccaccttgtaaaggaggtgccttgctttctactatgccctttctactatgcccttcaccttctactatgattgtaagtttcctgaggtctccccagccatgctgaactatgagtcaattaaatccctttcctttataaattacccagtctcaggcagttctttattgcacatatatgtgtgtgtatgtgtatgtgtgtgtgtgtgtatatgtatgtatatatgtatacatatgtgtgtatatgtatgtatatatgtatgtatatatgtatacatatgtgtgtatatgtatgtatatatgtatacatatgtgtgtgtgtatatatgtgtacatatatatatatatatatatatatatatatatatatatatgaacagagagagagagagagagggaggaagggagagagggagggaagcatggagaaagagagagtaatagcctaaatagaaataaaactagctccaagtacaggttcgtcaacactctcctatcatacccccaccaaagttaatgttaaccacttggagccctgttcttccttagttgtggagtactttagcaaaattttaaatctaattatgcctaattcaacgacagtgctaatttgaaagtgttagaaactgaagacctataataataatgagagttacaaaacataaatagtgagacaatgatgaatgtagtggatgcatgtacgagggctatcatttgacagtagagatgatgctcaaggacagacaatgagtctttcaatgtgtggagaatgtgctgctgttacagtgatgtacaggaaagaaacaaaaactgaggaagtatcagtaaacaaaacactcaaacatatgagtatacagctagaataaaagcaacagtactagatgacaataagcccaatgttaactcagaaagcagaaggtttttaagaatttggggaatactgtggctgatgatacttatgtctcaagccacagatgccatatgggctctgcgcccagttgaatcggcaccacctggcagtaagtgggcaggtccacgactgccaggacatcccttccaacacttgtggagatcaccaggaaggggggagagacctgccttgacagattttcaatgtgggcgaaacaggtctattttgagaaaagatgttcaatagaacatatgtcagcaaggaagaagagatgatgcttagttctaaagctccaaagagctggcttacactccaacttggggaaaatgcatccgggaaatgcaagattaatctcatcttagccattcttttgaatggatggacatgacccctttctacttgaagacagaaaacataaccatattgatttcaggttttcttcattggtttccatttaggattgttcctccccatcttctttctgtgtaggcatcccagttcccaagtgttcatgaagcacgtatggccttcaggggatgtgtctgtatacattgttatcttatggatgcacggttttgtctgcaccttggttctgaatgtctttactcttgagcatctgcccatgggtccccttctcaaggcctcaatttcttgagtttaacactgcatggcccatgcagcttttcagttaagcatctcttgctatgaccaactcttttcctcagtcaactcccacactcttttcagggacaggaaaaatgtagccacttgctggctgcactctgaggcctcaagaaatttagtgaatctgcctttgcccttcttgctgatgaaatactgccacatcaggccccctcttcggaaacctacaagcatctaattttcttgcttcctccccaactttctttttgactcccccccatccagagagttcttatgtctactgtactaggaaaaactcattcttaaggtatggttttcaaatcattctctggtctggactttagctacggttttaaatgaagaaacaacccagagccaaaatataatgaaactatttccttcttccacagagtggaaactgctttggggttaaagggccagtgaaccaaatagaaaaggatctcagggaacacagattgaagagagagaagaaaaaatatgaaggcattgttggttctcttttgagtttaaaatctagtggggattgtaagcacacacacatatacacacacacgcttacacacacacaccagtgaagttatgaaggattttgtcactccaacgaccttgaatttgattatctaggtcagttgttaccaaagtggaatgtacatgcccaataatatgcgtgctaaacagttggggtagtgagaaaaaatactttttatttatcttgttctctagaaattaatattttgattgtatattttatagtgtatgtgatgtgtaagttgtgtctacaaaactagtgtcaatgtaatttaaaattacatatgtctgtgaatatatatttatatagggtacatgcttaaaatgtgtttacttctgaggtacatgaacatttttcccccaggcacagaaagacaaataccacatgatgtcacttaaatgtgcaatgtaagaaaagttgaattcatagagatgtagagtagaatcatggttaacagaggcttgggaggtggagtgagggaatagagagttactgttcaaagattacaaagtttcaactagacagagggaatacattttgagatctatttcaggaacattttgagaccctcactctaagtaataggaaatcattactttagttaacatatttgaatatgagttgtgatgttctatatcgtttatttggattctactaacccacacctagatttttatggcattacctttttactcactgtgaatatcctactcatagacagatgccctgggaacttggacttgaggcacccaagaactgagacagtgagatttgggggcacaaggatctatggataagttcatcttagtgatgataaaatcaatttggcatgtttcacggacagtgtgcattttagaaagggtaaagacttggaaacgggatatttttgagcccaagtgtttccaataaatagctgtataatttgaagcaaataattgattttttgttctctttgtgccctcgcctgtaaaatgggagaaatgtattcctttctcatccttctcatgaggccattgagagtatctaatgagatcagactgtgacatagcataataattctcatttcttgaaggcctattatacactttgcaagcactgtatgtgttgtttctacttctcttgttcgtttttcctggaataaatatccccccctcctttacattggattgccattattcaccctgtaaggaaggcttcatggttctcattttcatctgagaaaacttaggctcagagaagatcagtaacttatctaaaacacacacatacacacacagacatatctatgcccattattcttaacctagtttctctattcaggagttatctctgctgtctctgcttctgattataatctgtgtaagctgatccaagtgacacgattacagggaaattgtaagccctttgagagcagagactacctattgatatctacattttaaaatttgattttagccaacctgtttatatgcaatgactaacaggttagtttgacttgcaataaatattccaaatcctagactaagtaaatttattaatgtaatgatttaacttgattttttcattggcatgtttccctgaagtcgtcatgcaaaattgaaaaaaaaaaaagtatagtgtgtgattctagattgaaattcaggaatcctccagggttaccttgtttgctttccaaatagttcagattgcttagtctgaccaacaaggtccctgacacttggaactctgtctatccctctaattgactttgtccctgatgacctcgcccagagatactcttcaccccagctatactgtgttgctagagtttctctgatatcccatgctattgtttcctttgttctcttcataaggtaccatttcccacccgccaactcctgttttcctgatggacttttgtttcaccttacaagatcattgctaatgtatttattttgagaataaaaagtgtaggaaaggtcacgggacaaagctgtacaccagacctttcccagacgaacctagtgtataatctccctagtccaacatcatggcttaaggcagtcgatagatccgtcttaatgtcccttttgagttttctactattattatatgaggatttatttttgtctgaattcctccctagatttgccctagagagcaatgactatttacagtttattcctctttgtatctcttatgttaaggccagaccttggcacatattctagctgattagaagacgtttgttgaatgaccaagtgattgaacaaatgaccatgtgctctgccacagtccggtcagttctactttggtttggttatgtgtttgccacattaaagttgtagcctgggaagttcagttgtgagatgtctgcagaacatgaaaaattggaataatgaggttatttctaaaattgctataatttaaaataaatagtggtttattccatatatgaatatacactggaaacaaagaatttctagaatactggagattcaatgataacatcattgaaattaaataaataataggattatgctagttactttctaatttactagaaattgaccgtgtgcatggcacgtataatgagtatcatgggatagttacaaaaagtggtgcttagtgagtttctgtggaaaatctcggtaccaataaaacggaggatttccagaaatcgatattcctcaaagcttgacagtatttatgcacggttacactttgtgtgtctttcgtttgaatcaatggaaggaggctataactgaaaattattgttttagtgtattatatctttaataataagagttttaagaatctatcattagaaataattattcctcaatttgtaattctcaacatttgaacaaataaatgctctgtgtctatcagttaatcttgcccatgaagatttaataaagcacgctagtttttacaaatgtgattttagagatggtcattacttggtaaaatattttgtgttaacacttccatgaatatgttctgtgggaatatactgcctccacattgcttgctcatgaagacatgatttttcacatcatcctatcagtattttgagaaagagattgatcccatattctatgagcatttgaacattctctagtatttttgtttaatcattaaaacaacccttgaagtctatgtgctacactggttatttccctcttgactttcctttacagataaccctctatcataaacaacctatctatatttgttgtctccacatcatgttgccagccctgctttaacacactgcacattgacttctagcagcaaaggctcatgggaggtactctcatcaaggacactgatggtcctcatgttgctaaatttggtgggtcctctacagtctttatcctagttcaccttattatggaccactgtcaactctgttctgcttaaaacactctgttccttgcttatatgactctacactcttaactcctttgtgaattcctcatctgcccttccattaagtattgacgacatccttcatagttttgatctaggacctcttttcctcttacttgacattatgtgggtaatcttgtctttgaacgcaattaccattcttatgttgatgacccttaagctataattccagcccaaatcatttttctgaggaagctacaagaatacacaaatgtctaatagatctctatttagatgtccctcaggtgcttcaagcttaaaatactcacctgagctcatcacctcatctataaattctgcttctcctccctggctccctgatttatttaatatgaccaccatccacttagttgaataaagcagaagcctggacaccatctatacctccaattaatcactaagttttgttgttaaatacgttcttacattttctctctagaatgtcttattttccccatctttacacccaaaaccaaaagtcagatgaccctgatctcctgcttagatttcaaaacactatctcttgcctagactctggaatttcagtcttgctcctctccaatctatttctacaccctagactctggaatttcagtcttgctcctctccaatctatttctacacaaaagctagagtaattttttaaaaaacaaaaatctgaatgtgttcattttctgcctaaaagccttcagtaattcttatttgttcttccagggatagagtaacaactttcagacctagtttattagctagttctttaaccacaaaggactctctcacttgtctactccccctaacacacttcgccctaacctttgccattcctccctttcccttttccttcccagatggacttaagtcctttcagattcttaaatgtttcttcctccagtctcttacatctcttttccttgtaactctaaaaactacttagcttacgcaaggaaaaaggtctgtacaattcccggaatcagcgatcctaacgttccctgttgtttttttcgttgggacatgaattcattcacagtggctctaaacatcaccacccctgcctatctctcccattcctactttatctgagcttatccatactcttgaagacttacatattttttttctaccaggaaatcattactagccttattatcccactgtccaaaccaataagtctgattaggtatctgtatatatttaatattactatatgtgtttttctaacactctagtagaggagaaggtgtatttctttctgttttttagaagcctgtatttctgctattatagctcttaaggaactctcatgcaattgcctactagaatgtaagttacggtaggataagaactggatcagtcatatcacacatccacatataggacctagcaccatatctaacacacagcaggtactcaatacatttctttcccaaataactaaagagtttaaacaaaccaaaatgattaaatgagaagtaactgttttggtaattcttgtgtccttactagagtctaaattgagtgatttttatatcatcagtttatactcccctttcccaaccccaattctttcttttttaaattttttaaatcaaatatgccttaaaacttcaggatcagttgagtaaaatgatgcttttgtcgtcttttgcaaaataattgtatttcagaattttgatttagatattataaacacacctaaaataatagctttagtcttaagatgaagtgcttcttaaactccctaagatgggttggactatggatatgaacatggacaatatcacattaatttgtgtacacagttctaacacagggtctggcatataagaacaagtcagtaaatagttgttgaatggaattgaaaatttaagtagcaaataaagtattttgacctacaaagcaagaaatcacatttttctttttgtcacagttccttaggaagataattaattttttagtatttaaggatgttaaatatttattttatgttctatttactaggcttctttttatgaaaattaattggtgaaaatagcgtacatatcttcctttaccagaacatttacattttgggcagtaacgctggcttttgttaaaaaagcaaaatatgtgtgaaatttatgtttgagttgatttcaatgcattacatttccattttaaatcttctttgaaatactctatttttgacaccatgaaactgtattagatcttagtatgttagcaatgttttgcagttttagagccataattattttaatgaccactttcagcatatacgttttctacaggaaaaataatctcaagaacatgaaaagtgaaatctatattttgggtttcaaaatgatacattttagctaaaatatcatagttttaatttctcagtgaaaaatatagtgtggtaatttatgaagagactcagtgtttaaaaattatgactctatagtcaagtttatgtttataggacataggttattcaattacatttaaaataattaatttagaaaatgtgatcaatgtaacaaattttacctgttcttttctaaagctaaatttgttgtttgaagtgtttcttctaaaatgctaatgaactatcaatttaattgttgagcttagagttagaaacttaattatattgccagaaataaagaaacaaatggatcccaaaagattcacacattagaaatgtatgccagggaaatgcttttgaatgtgttcaagtcatggcttctaactcgtaacttataacttgtgttatgtctggcttcattcccttaagaaaaaggaataataatgccttcggagagcatcccagctgtaagagctatgcattggtgtctaaaaaagcttctcactcctcataccatcctggtctgggaatttaaaaaattgtcatcttttgataatctgtatcacatagtcttctgcatagtcatatgaggttagaactgccccataacttttgcagggcctatagtaagtgtgcaaatggttgcctgcatgccacatatttaatatttataaggtataaagtcaacagactattaaatatatcctatctgctttccttgacaattatacaatcataatgatatggacatctagattcgatttagaattctctctctctcattttctttttcttctttctttctttctctttctttctttccttcctttctttctttctttcttctttctttctttctttctttctttctttctttctttctttctttctttctgtctgtctgtctgtcttgtttttttaaatagtgcaagcagtttattccctggcaaggaatttggaaaaaactcaaatagcaaaccacttgatacaataaaataaattccttagagttttgtactggaatgaggcagcttggttagagctaaccctaagcctgttatttaggatacattggcttttcttaagcttaaaaaaaattttactgtgttaatgacatttaacatgagatctatcatcttaataaatactacatgcacaatacattattattgactctaggtagaatgttggacagcagatctctagagctaattcatcctacttaactgaaatgtaatgtctgttgattagtaacttcctatttcgccctatccccagcccctggcaaccaccagtccagtctttgattttatgagtttgactgttttagataccttatttcaagtaagtggaatcatgcagtatttgtctgtgtctgtcttgtttcacttagcgtaatcttaaggtccatccatattgtttcatattgcagaatttccttttataaaggctgaatagtattccattgtgtatatataccacatttatctattcatctgccaatgggcatttaggttgtttctgcatcttagctattgtgaatctgttgccttttttccctacctcctttactccatcctgcactgtgaggaactctgtgcacatagatctggtcgccccatttcccacccacatgttcaagtttttcccactcacctcatgcaaagatttaccccttagccatacccagtaactgactttgaaacatttgcccagggagttgagggattctgaatgccagatcatgggagcggggcttctagtgagcatgttggcttggtcctacagactcctaatcagagctttgcctttgaaagcatggggcccaagggcaaggaccctacttgttaaggtctaaattttttttctgaaataaccacatcgagcttttatgtgtagatggcctaaattgggctaacccagaggcagtgacactcaagtagtttacatctaagcgctttccatgtgcttcttttcccatttctgttacttcttacaaaataaaaaatcagcatctcaattaccctgatttgatcattgagcaatctaaaaagtatcaaaatatcacatgtagcccccatatacatacaactgttatatatcactataaataaatatatacacattatatttaaaaatcaatactttaattttacatgtttaacaaatcactagcatatacattccagattgaacttacgagggatgtggaaaagattcagtgactaaataacaataaagtactctaaaaatgaaaatgtgaaatggagacagtataaatctaaaatcatatcacttatgaagtattgtttcaaataaacaataaaatatatcttcaatcaatttaattttattttagttgtataaaatctttcggtcagcattaacctaattggaacactaaataggtacatctaaaaaatataatcccccccaaaaatatgtagctcataagagataatgcattgaacacagataatattggcgttaaaaacagaactctaccacatttgcaacgaaatgtttatctgttcttcctactagaaaataataaaatagttctgcatgagcttgaactcgaagtattaggtgtacaaagaccttttagtgaatgaatgctagctgaaaagcaaattttaaatatgaaaaattagcaagacaaacatttgaatttgtgggagatgagtaaaactcctataaaaatgaattgtttagtgttaaacagattgtgtatgaaatattaatggcatattgtcctgagctccccttccgctgtttccatgtagatgactgaatttcaaacagaaatatgccaggaatgattacgtgaatgaatattactacatgagattgcttaaagagtatttcttcttttgccttctttttactttcgttatttcatttagtagttagaaaatactgtctacaaatatgtgagaactgcttaatttatttttgagacattaattaattcaactaaactatattgactgtgtgagagagattcccttggtgaatatgtggatttttgcggtggtaagaactctcctctggagcgcaaatggtattgctctaggaataaagcatatacctcaggcccagatgaaccagtgcaatctacagtaacaggttcaaagatgacctcatgacctactgtggactaataaaaatcaaggagacctactgcaaaggtttctgggaaattctttttctcttgcgttgaactaagtaatatacatatgtgatagttagagctgcagcctttgtaataccatgacagaagataacctgaaataaggctgacagacacaagagggagacctaagagtactgagagatatggagcaggaccccctgattgaacttcacttgcagcccccttctgcagttttcaatgacgtgaaccagtagaatccctttgtttactgtttttgattaatttgagtgcagctttatgttatgagcaactaatagcatcctcactgtcacaactgccctctatacggcaggcactttgtgatactaaagaaagcagtatacagagtagagcccagtgaataacagggcagatgttgcaattaaactgcctgtttaaattctagctcttccactagctaacttgtgactatctaagtaatttaaccttcctataatcatacctatcttgaagacttgttgtaagatttaaagcacaacagtgctactataaaacaggtatacagtaaagcttagctacttttttattaggccatatgatatcatttcattaaaatcttatagccatgctataaggtattatgatcctcaatttataaataagacagctcaagttttggtcaagtgactttaccaaggtcatagagctagaaaataatgattccaagttacaagccaaacctcttcaatgccaaatttacatcatcccccattacttgaagtgtaagattcacatggacagaaatttttgactgtttgatcactgctatctccttatcatctaaaacagtctctggtccatattaggtgttcaataaatatttgtagagtacataatttccttcacagactccacaatctggtgaaggaggcagacatgtaagagaattatttcaggattccacagttgatgctgtaacagagctaaatataatgaatggaggaggaatgaataagtttgtctgggagcaatgctatggctattgaaataagtcttgctcatgctttgattgaaatggtggatatagatcacacaacaaataacaattagataacagcttgttgggagaaagcgaggatcagtgtttgccataaacatttctcatagctaatgtcaggtgtttgatttctcaacattttatatctttgactttgattttctctgtttttattttttaactccattctcaagaagtctgcacataagagtttcaacatctagcacttcataactccgtcatctcctctcaggcttagagcaaattctgagacgtggatttatcgtcgagtgatttcttcctggcattttatctctgagaccaggatctggttgctaagcatgtagacatagaaatgcatttcttcattgaaccccataggttcaaactagtggataatgagcacaatgtcaatgtgattatttgtaatgggggaaaggttaccggagaatattacacgaccatccacatagactaacattttcctcatgactaagtttacttagcaaaacaaattaaaaacagaagtttgtttagcagcacagaattgaaggaagacaaccagatggttatgaggaagattcatccaaactatgccagaactgaaagaaattaagttcattcagtacaagaattgtctagaataagagaatccattttgtgtcagcacttcccaagttcttgttaatgctaccttaagttcaattcaaaccaggcagcatttattacgtgttgtgctgggtcctaggaggaccgcgttttaagaacttactgtgatcttctagatcaagtttttatttcaatatttctacctcatttctgattcttaggtgttccttatttcccaatttatcccctgcagaaattgaggcaataagatgtctatcttattgcctatggtgttgattatttatgttatattctgttttgtgaagtttgacctctacctaattaaattacattttcaattgtatcttggattgatttattcaataagtattctttaatatttttgcatgaggtcggtcaggtttcatcagacattaggaattaattataaaaatctctagattggtacttggagcttaaaggaataaggtggtggaacgttaaatgaggaggaaagaaccagcagagctgggataaaattcatctctatcatcttcccacctgcttgatctctggcatataatttactatccgtgaacctcaggtttctcttcagaaaagctgcagggttgttgggggaaataaggcaattcctgggcttcagtatgttcaaaacagagcattaatattattatagacttttgatgatttacacaattttagctttttggcaagacatatttactagtactaagtaaaagcacgttgactttctaaaatgaaaatgtgtatgtgaggatgaagaaaaagaaagtgttttgtttgataatatagcattataacactgcacaaaaaaaaaatggtatatgcagagacttccatcacttgcttatgatgccgcattgggatctcattaataagacacttcctcagacacttcctttgtgttcaataaatttcaatttcctcctttccttcagttcacttcaagaaggacggcagcaactttcttgttgccaaacctgacaaatgttttttagtgctgattatactcgagcattctgtagcaaaatgctgtgggtgaaaatgccttccttcttaagggaatttagcttctgtagtaccagaatctccttgttgaatgaacatgtactgcctaagtcttagtaatccctcctttttgagcccattttctggcatctctccctttaatattcctcaaaaagttggatttttcctggacttttcatattacagactttcctttggtcatcctcatccattccgtgattccaactacattttccctccatcctggcatcttctttcttccagacttgtatatgcaactgcttccattcatacacttgaccaaccttttaatttctataagatcaaaaactcagctcacaagctttcccctaccatcgagcggggttcttcttttgcttctttgtttcagacaatggcaccaccatactcgagtaaggcacgttcatttatcaggtcctaccaaatctacaataaactctcttgaatttatccacttgttttcatttgaacagtcatttctttacctgggtagcctgcaccttctacctgcattgattcagcagtctcttcaccactggctctccctccctctcctgcctctcttcttgctccttcaatttattctctactcttcatagtgacttttattaatgcaaatatgaccttataactcccttgcttaaagacccactcatgtttgtctttgtatccataacttccggcctagggcttaacgcatagcaggtgctcagtaaatctgtggtagatgaaagaacaagttgtataaatactgaatggtctgatgtgctctttgttgtgtcaagaaggacattttgcagtcaggatagctacatcagtcctttagtaggcatttgacagcactcgcattattcctcaagagaagatggatgtattgattctgtatttcaaatgacataacttttgtgaaataagaggctgccacggtaatctgagggatctctcaagttcaagggactccacagtgctttgtgtaaggtaacaggctaaagggttcagtcttaaactttcttaagactgtagttcagggttcctatggtggggctataaccctgaattacatcctctttcatttcatgctgataatgagaactacaaaccaaggggtattaggaaagaatccaggtttgatgcagggaaaaataaaaacaactgataatctctagtgtccccaacttcaagaattcctttcttctttacaccaagctttttttctctgccaggacttactttgtcttctacatgtttaagggagaaaaatgagttaacagaaggggaggtacagcatttctatttacttagatgctagagaacaggatgaaaggtatgaaaaatatgaaagtctctctctctctctctccccagccttcccccgcttctctctctctctctctctctctgtgtgtgtgtgtgtgtgtgcacgtgcgtgtgtgtgtgtgtcataatactcaacctttcttttctttcaagcatatgttgtggcagagacaagtgtacatcaaaattcgtggtccctctttcatagtatagagttcttgctaggatccagctgcaagccagcaactacatttcccagccccactggcatctagttagagccatgtgactagttgtgaccaattgaatgtgagtgggagttatgttgcaggcataccttttccatcttcttacttcccatttgctaaccttatggaaaagagtcccaaagacctaggagatgaaaaagcctaaaatggaaggactcagagtccctgaattactgggtagagaaaagctgtttgcagatgggaatgcccattttgtagtattctttcttttcttaagccactaaaattgtgggatctctttgttatagctactggcattaacctcttacgtatacatacagctatgtgctacaaagaggaatagatacattttttaatcgttgaaaggggagaaagaaacatatttaggaggaaaataatttagtctctacaattgaaaagtgttttatgaataatattttgttttggcagcatattaaatctcaggcagctgaactacattaattttcaattctctatatatgtttttgtcttcagggtttagtaacactgatatataacagtttctttcttttaatttccaaatttaaatgtctaagtttgccttctaggcagaaattaagtcccattgtggaatgagattggatcaacacttcaccaagatcattttagttctttgtaatcttaaatgaaataagctaataaagcattaaattagcatgttgtaaaacttcgtgaagttttaatatgcttctaagtggcagctcttagcttattatctctaaagctaaagtcaaaataaatgtctcagttgatgaaatggagatgaggcaacattttatcaaatttaacaaaatattttatatctgaattataaagtccagattatctagtaattatcatataaatgtatttaaccagacatgcatttttctctaatcagtagccctggagtctttggaccacaaatgtgccttatctcaaatgctttaactgtgacattttgctttagactagctcgactacttctacagaaattatacacttcattcacattcatccagatgaaaaaaatacatgtagaaatgatcataataagtaacatttgtttaggatttcagagtttacgaagggtttttctattcactttctcacttgttcttcatgtaaactggtttggtggacaactgtcattatccctgttacctggagcccctgggtcttagggagacttcttgacttctcaaggtcatgaaggtgctaactctgaccgtgtttttattcctactgtgccacacttctcaggtaaaaatcatattgcagacactttaagagaagtacttaagaaaataaattcctccagagaattacatttaagttgtttcattaactgcagtgcataaagaaaggaaaagtgttcccaaacccatgtagtattttgctattgcttatggtaatattctgcacacctaatattgtcagcataattttccatgtaacaaaatgtcctaaatcagcaatgtccaatataactttgtgtgatgataaaaatgttctgtctctgtgctgtccaatacaacagccactagatacacatgactactgagcaatggtaatatggccagggacactaaggaactaaatttttatttaatattaaataacgtttaaatttcaaaagccgcatgcggctagtggttgtcatcagatactgcagttatagaaaattagaatttacctctttaaatactaaacctatttttaatagtaggatttttaaattaaaatagttctaagtgcttttaagtgatacgaagtcaaatgcaagatttctgttttaatagtactctcaacccagagacaatcttcatgcatccttatacatgttctttgttgccttattctagttttattttaacattaaatgcctctgttctacttgatattgacttgcttcagagaacaccaagtatagtggaaagaaacacacacatgaggacttgaggctaccaaccaggttcaactaaatgcactctgatttaattgtagtattgggatcccctgttgcatttattgaagaagaaaaaaactttgcaaccaaaaagatatttgaaagcaactgttcttcttggacacatgatccctcataaagtggggcttcctgcttttcagagacttaatttctgttcatattcatttcagcaatagtaataatgatgatggcgatgatgataataatcatgatgatgcctaagtgttgtagtaatgcttcttctgagccagacgttagtcaaattactttctctacattaattcaggcaatcatcacaacaatcccacaggacaggttttattattatacttatttagctagcaaatgatataactaggttaagttacttgcccaaggtcatactgccaagacagtggctctagtgtccctgcttctgaccatatgttatgctgcctatcctagagcttttctcttctaaaatagtaaaataatatattctttgtttgtttcatacttttttttttttttttttttttgagagggagtttcgctctttcgcccaggctggagtgaggtggcgcaatctcagctgactgtaacctctgcccccaccaggttcgagtgattcccctgcctcagcctccgaagtacctgggataataggtgcccaccaccatgcctggctaatttttgtgttttcagtagagacagggcttcaccatgttgaccaggctggtctcgagttcctcagctctggcagtccgcccgccttggcctcccacagtgctgggattacatgcatgagccactacacccggcccatacataaatattttaagcgaagtacacatgcatgatcatcatacttttaataatttcatttaactgtttccaaagaatgttagtatgaggttttctttttttctttttataatttcaacttttattttagattcagcgggtacatgttccctggatatagtgcatgatgatgaggtttgctatatgaatgatcccaccacccaggtagcgagcatggtaaccactagttcttcaacccttgcctgttcccttcctccctccttcctctgtagtccccagtgtctattgttcctgtctttatgtccatgtgcactcaatgtttagctcccacttttaagcgagaacatgcagtactcgttgtctgttcctgcgttaacgtgcttaggatagtggcctccaattgcatccatgttgttgcacaggccatgattttgttagtttttatggctgtgtagtattccatggtgtatacgcgccacattctttatcctgtccaccattaatgggcacctaggttgattgcatgtctttgccattgtgaatagtgctgtgatgttatatgtactttttggtatattcaaagagaaatgctattttcctcttgacatatttatgtcaatttaacatatttatgtcccttttctttttaggagcaccattctcttcctttaacattataaataaaatattttttgcttttctgtttttgtaagtgcagttttattgacagagtgagacatacacgtcgatattgtgactagctgcatgtcttctattatttagaggtctcactcaaatgtagattatcaaattctgttagtgaagagggtagaacagcagaactaatgctggtttccttctctagcattatttgatgataaactaagatgataataccccccaggtcttagatacctgcagtaggacaggcaccctacatttaatgctcctaggaatccttcaaagtgatagcatagttattatacagtaattgagaaaactgatgttcataagttagaaatttttccgaagttgcaaagaaagtgaatggaagaattataccaagttctggccgggcgcagtagctcatgcctgtaatctcagcgcttcaggaggccgaggcgggcggatcatgaggtcaagagattgagaccatcctggccaacatggtgagaccccgtctttactaaaaatagtaaaattagctgggcgtggtggcacgcacctgtaatctcagctactcgggaggctgaggtaggagaatcacttgaacccgggaggcggagtttgcagtgagccgagatcgtgccattgcactccagcctgggcgacaagagcaaaactccgtctcagaagaaaaaaaaaaaaaaaaaaagaggattataccgagttctctttgattccaagcccaaacaaatccttttttgcaatatatgacattgtttccctgtttgcattccccattctgtgtatcacacatcctgtggcctgatcaaaattcattttcagattctgaatttattttccattgaatctatataaactataaagacagaagatatatgtatgtgtgtatacccacgtttctcttccagtgtcaactgataaaaatagatttcaaagtctcaataacctttaattccctttttctcttaaaaattctttagaacttgtacatgacattctgactctagcagattttagaaaacagagaggccattagatattcataccttactattcagatgaagtattcaatgctaaattatgtaatttatctgctttgcaaattgtatggtcagattgagttccacaaaggagagataatttttaatataggcattctgtagcttccctaattattgaattagtttagagcaaaatccttaaattgtatcgttgctatgctcaaattttgtatacttgtccacgtaggctatattaagatttcattgaattttggtttctttctcagtgataattcaatatatcaactcaccactcagatttgcctttgggaaaatccaggccccttttctggatttttagagcagattttaaaaaagtgattctgtatatgtgttgaaattaaccacatctcattgcttttgaatgattgaggtaatgtatacctactactttaaaaaaaatgacttacttagaaggtgtccatagttttataagttccattgaactggtttatattgtatttagaaaggaaaactactccttttatccttaagggtgaaaacctggattttattatacaattaacacatatttattttttattatgaaatatatcacaatataaacgtttacagggagtgtttaaagtggtgttgtccaatggaaatataatgtgagtcaaatacgtagttttcaattttctactagccatattagaaaaagaaacagagaaattaatgtaataggatactttatttagcctagtatatccaaatcacaattatttaaatatgtaatcaatataaaaattactaattatgtatttaacctttttctttagtaagtctctgaaatctagtgtatattttacatttatggcacattgcaatttgcattagtcacatttgaattgttcaatagccacaggtggctaatggctaccgtgttggacagcacaggtttaaagaataatatgaacatctgtgttccaacattctgagtttcaaataagaagaacaccatcagtattttgggagaagctccctatgttaccccttgctaatcaccttccttccccccagagccaaaagtaaccattatcttgaatttctagtaaacaatgctcattttttaaaaaacgtatgttcaacacctgtatttgtatctttaaagagtagctagttttagtttgcctggatttgaactttatattaagggaaccaccccatctctaatcttctctgtgaattcttttctctcaatactatgttttacatatttacgttcatcaatgtgcaactcattgtatgtatataacacaatgtatatattttacatgcgtatggacatttgggttgtttttatgtttttgttcatcacaaaccacaacacacatgtgttcttgtatatgttttatagtgcatgtttaaaaatttctcaacagtattcgctagtagtattgtcaggtcatagggtatgcacacataaatagaaatgattgattagctgcaatttgtagtgcacacatatttgctatgtaagtgatccatgtttaagactttaactgaatttaaaaaatattttattggagccaatctaaatgagctaagggtttgtattgtttacataagcaaagattacacttactgggtcaattcggttgattaactttggatatataaaatatatagctagttgttaaatagatataattattaattggcattacttttgtttgtatataaaaatttcaaaatatccatgacttaagcaaggtaaacacccactgggtggcttaagcaacagaaatgtatttcttgcagttccggaagttgaacgtctaagattaaggtgatgacagggttggtttctggtgagtcctcccccattggcttgcagatagccgccttctccttcatgacctttcctctgtgtatgtgcatcccttgtagctgttcttccttttatgaggacattagacttattggattaaggtcctacccatatgaactcatttaaccttaattacccctttaaaggccctacctccacttgcaggggttaaaacttcaacatatgaatggggttgaggagacctacttcagtccataacagtttctatattctgaagatggtctttaattaactaaacagttaatgttactttactgggaatgtcttttggatgggggaataagctgatgatatgagaagggttggtgaatttctcataagtgtgaaatttgttgggccggcccagcatgattttcaatcaaatacgctttggggacaagtaggttgaatcactacgagaggtttaaaagaaagcaagttgtaattgcaacttttaattgaaagaaagacaggctttgttgatgtgccagcaagactgataactggctttaacgtagatagtaaggcagcagattcaatccactgatcgtgatctactagtgaatttcaaagccttatgcaatagaactacaaaccctttccttgcccaccttgcaggtggatccataggcaaaatgaacatttgcaaaaaagccgctatgtttcagaatttgtgctagggctttaatatctataatttctccaaatcctcacaatttaagaattaattcaacttagccccatgaatagggtgaaaattctgagatttaacaaactaaaataagttatctgaagacagacaaatagaaagagttgagatattctatttgaatgtaaaattttcaaaaagtagaatgacagcgtcaggaattacagtctcagtgttgaacacaagacttaggaacaaatttgctgcatgtaatttcattgagatgggacaaagtacagcatacgtaaggaagttttagaacaaataagataattattttacgagctttgaaacatgtgtaagaaagatacgaataaaagtataatcacatttgactaaaacatgaataccttaaaactgaaaagcactgagattatcattatataattttgaatattttaaaccacaatgctttgggagtgcactgtaatattttagaattggaattttaacttactggcttaaaaagtaatgtactttgttttaaattcaaagattatcttgtaaattcagttcgatctattgaaaaaattataaaattcggcaagaagccaaagaagaacaattatgtagctcaagataattaaattttcatgtttggctttagaaatatattcgtcgtgacatagtacatggtaatctagtgagcccagacaagtagttttctctttttgtcaaagggaacaatttgatgcgtgttcaagttgcttaaataaaattttgtatgtgctttctcatcacaagagaacaatatgatttttgaaattatttttactttataaaagaaaaaaaaaagccctcacagagaaaaaagaaaaaaatgatgatgtctttgaaaaacaaagttaatacagctttacatatatttgacctacatcagggttaatatttttcaaggtgaaacattagatgctggaacttgcaaaaacaggcaatcctcctttagatgaaacggacactctaagggttaattcattcactgagacctattgtgaagtaagccctacagagactgaaaaagttaaatgcaactcacaaaagttgctagaagagtcatgatgttaaaataaaataagtacacaatgtatgctgcaagtatacttagagccatgctaggtgcggttgagaagttcaatacaggtccaagataatagctgcttctcctatagaacatgtcttctcattggagggataagacctgtgtctatgaaacaggcgtaattacatagctctggaactatatatgccgaaataaatgagacagtaagtgttattgtactataaagaatgaagaaatcatgatgagaagtaacagttaatgaatgttttctagaaagagtaggatctgaattggccttaggttgtaagcagagtttatagatagagtagtggtatgtcagagtcactctgggtgcttaaacatacaaatccccaagtctcacccaaatgtgtcttcagatgaaaggaaaaaacaaatgacttgagctcccccgcaaagaacacgggtggtatattgagcagccaaggagtgaccagagtggcaggcccatgttgagggacaaaagaggacaattagaatatgattaatacaaatttacagtgggatgagttgttagcctgaggagcttgaatgtgaacctctgtgcaaaaaggagtcattaaatacttttgaaaaaggtgggatgggaagaaaatgacattctcaagacaattagatcgaacagtattaagcatgctgacttattaagttatgcaccttgagagggtggaatgagggaaaagggtctttatctggagtaagacaggaagaagctaagctgtaattcttactggactgtaaattatgtgcagatatattatctgtcatgttcgtgggcgcattctcagtacatagcacttgaaacaggtactcgataaattgtcaaatggatgcatggagtgatttccatgcaaaatctaatattgtatagtattagaagggggaaaaaagcatggcattatgctagcagaaatgtcatttggtattgaggatgaaacattttcaacagtttgcaaagccatccactcaaacattctgtcactttccaataattttgaaggatgttctttctacttctaccttattacacaatgagttgagtaagataaagaagtcatgtgcaacaaaacagagggagattttctgaaaggcactacaccaggaagttgttgtactcttgcttcatcttgccatcttggatatacttctggcgctacctccaggccagttcctcgttacatatgtcatttacttcccacatgctagactcaccgagttaatcattttgctgcagttaacacattttagcagagtgtaggtttatgggtgagaaggaaatcaatgatgtttcaatacagggttcttttcccatcccccttatttccacttagaactgtctctcaagtcttaatttgcctctaaacttttttcccagcttacattcttttctgaaaaatgcaacgacgatgccaatgtttgttgacctgaaatacattgtaaaacattcataatactttgagcagagcttccaaactcccatttgcctcttttatctcccttaccttggcccctttttgaaggcaatgtgatatttaatccgtttctattgatgcttcaaaattattgaaaaactggtaattgtatttttccctttacttatcagttgctagttgacaatgagtgtttgcccaaacaataaccaatcaaaaggtaaaaaggagattccagacatatctgagaagaaattctttggaagaagcccgtaaatggaatgggaattcaaacaaagccgtttccaaaagaaatactaaatggtctctaaatgcaaaaggattgctccccaagcattttatgggagcataaaaagctcccaacacattttatgacaatacttctactcaatgacttcttgtgttgacatatttgttgcactcgacgttagtatttacagcttcttatcccaaatatttacttaactgaagccctgatgtttttaaaaacttttcatctgtgtttaacagcccattttacagaaacttatttgtttcatcaggcagatatttactgagaacttgcaagtgccatatattctaaaaatgctgatgataaaactgtgaacacaatagattctcatggtgcttatggtcagggctagcacacacacttgtgaaatgatcactgatgatcaaaggcataaacactacatttggaagaaataccgagggatccagaagtatcttggaaacactagcaagtatagcagatggtgggattggtgcttcaaagaacttcttgtggaagatgttacgtatgtaccttctctgtgccaggcactgctaggaagtgctggagagaaaaagatgtgctagataccgcctctgtcctatgtgcttgtgctttgtggggaggtgagtaggataatcccagttctcatgcagtgtaatgagtaccatgacggaaatgcactccaagaactaggcagcatgaccagagataggacatttgagaaagacttcactcgggtggtactatcttagtctgggtgctaaaatagatgtgatagatgagtaagggtgacccggaagcaggagggaaagggaggggctttcagaacaacaagtgcgaggacattaaggtgaaatagagtataatagtattcccagatccttgggattgttctccattaggctaaaacaaaggtgttttctcttctttaagatttcatgactgcagattgcataacagaaggtcatttaatagacctctaaactgaaggaattcttgaattaaatcacaacatatcttccatggccagagaaaccattgcctccttatgtcgacattactaacagcaccagcacctgctgctcaggccagcgggagggttgggtgttgctgcctaggtaatgctcaccaactgatgtcctgccatgagtagttttgccaagttccacaaaaaaaacttagtgttctatcagcatctaatgagaattacagtcattagttaaataaaagaactattagataaggagcagaatgaacaacacacaatccatcagcttggtgaatggtatcagatggtttctgggtgctgggcagctgtgcatccaagtagacagggagaatatatatgtcctttgccttatgtacttgtttctctaatccaaaggcacagcaatccgtggaagctgctatgataaggtgtttagtggtgaaaatgtcttgaaagccagtagattattaaagtgatgtttttaaaaatgcagatggagagtaagtactttttatctagagtagtagttctcaaagggaggtcccgggatcagcagcgttagcatcacttgggaacttagacctgcatgggccccattccagatctcacttgaaaactctagggggtgtagcccggcagtctttgttgtgaccagctctccagggggttctgacactccaaatgttcaagtttcagaacgctactcacaggccatcatgctcggcatcacctgaaagcttgttagaactagaaagtcttggccccaccccaagcctactaaatcagagtttttgggagtagggccaagaaaactgtgggttaacaaggtctccaagtgattcttattcatgtcaaaatttgaaaagcgtcgatcgaactgttggttctcagctttgattgcgtatctgaatcacctggggagacagttgagctattccgggcccagatcacatctagaccaattgaatcagaatctatggaggcaggacccagacatcagtattttaaaatatttcttgaatgatcccagagtgtagctaaggttgagaaacactgttctaggattaaaggattaatgtgtttgagagtatgttaagatcttaggcaaatcacaagggtgttaagaactaccatcttcgcaaaaggagaatgtgcctcagatattctggtactgctttgattttaccttcagtagtcttacctattttgagtatgcttagtagtactaatatgaggcttattactaatatgttaaaatttgtcttttaattaagtgggtctaaacgttttaatctttaatctctgacccaactagaacttttctaaacattttcataatagtctccaccttgtcttctgaccttcacttatgttctttcagggttcttcgtgtgttactagtaatagtaatggcaagtgtttattgaacacttactatgtgaagattctaactggcttttaataatcacatcagctctgggaggtagaaggtagggatcctccttgcttatcaggtgagaaaactgtactatagagaagttagcaacttttcccaggtcataatatgtgacagctaaagggagcataatggttggaataaaataaatctactctagttgtaccgaaggctcatatttgtctcacgtacttgatttggtcgaggcccaaggggtcaatttccaatgcttggattcctggatatgtagagttgtattaaaaatgctaaaaacctattatgtatcatacaatcatacatatcacctaaagtattatggaaatgaatctgtattattaagggaaaaaggcctgtgtgaagaacaactgaaacttcattttaattgaaattaaataacatgcatcatacactaaaagtgcacgttatgaccccatgaattacttcaggtggctttgattcatgttacatacactaacaaatatagaagagtgatataatgcttcttaattaactactaatggaagtttactatttaactgcttcttatgtaagaatgtaaatgttttctgaaatatcagaacttttcattaggaagcacttttaaaaatagcaaaactgatatgcactatgatttccatatacattaaattgaacttgtaaatgatgttataaattatagaaaccaaggggatgttcaaattagatatttgtctaaataaatcatgtatggattgaacaaatactcattgagaaataaatgtattccttttctttcaattatctaggattccttgtttatctcttcagaagcaaaatgtcttctgtccgttttatttccagttaaacattcttcagattatgtaaataagttaacttccaatcctcttatttctgtttatctcaccactcttctaatttagacgtgatcaatatcttatctttttgcatttcatagacatcaggatccagaataattgagtgagctcaaaacaacaatggcaagaatgatgttttcagaaaactcagcaatcattcgtttaataaatattcattgcctaccaactataagcaaagtattggctaggccatgtggggtatacaaaaatgtattaaatatggctcattctccctaagaacttacacctattagacaaagtacatgcataaaaattataatgtataatagaaaataaatacaagccctagaatgcacagttgaagtacgatttgcatttattataaaaagaaagatgaattggctgggcacggtggctcacgcctgtaatcccagcactttgggaggccaaggtgggcagatcacgaggtcaggagttcgagaccatcctggccaacatggtgaaactctatctctactaaatatacaaaaattagccgggtttggtggtatgcacctgtaatcccagctacttagcaggctgaggcaggagaattgtttgaacctgggaggtggaggttgcagtgagccaagatctggccattgcactccagcctgggcaacagcaagattccatctcaaaaaaaaaaaaaggaaagaaaagaaaagattaatttcctgttagctaaatcaaggaaggcttcatggagaaaaaaatatttcaacacacacttgacgtagcagtgggatcaggctgatgttagggaagaatgaatgacattctacactgagaaagagatattcagtatatatatgaagagcagtagagaaactaacaagtggaaatagactcaatttacaatacttgcctgcctggagtactctatacgttgactgtaagttgcagtttactcagaacaatcccactttctacttgtttatcctatgtaatcatttattgggcctccttttgctctcaaaaatatccttgtttggataatagattatcactctgttcctaaatgaactgccctgtgtcctatcccagtaaaagggtgcattcgggcccttcgtaactgcctccactacatggttgattgaaaccagagcttggcattaagaagttagctgaacaatcagatttctattcttggaaaacccaagaatttcagaatagatacagaagctgtatagctttaataacatgacagagttgtagccttgaaagctatgtacaattcagaattatgagggagaagaaattgaagaaacagtagcagccgggtaaatgcagaaacaaatgagggagacacctagggggtgactgaggcacaataatggaagagaagtgcagtgaaattgcttgaactcttactgatgagatttctactgttgccttgaatccaggaccacctatatgttcattctttgtcatgctcagagttatgacagatgctgttattgaattccccagagactcccttatcgtctcacctcaaaccttacaataatcccttctatctttctatccatccaagctggcttaagtaaagtctatgatccatattcctagtaaacagagaagggaaagagactgaaggcaaaggccccaattagtaggctattgcaatatttcagggaaaaggcaatggccatcacattgttgtcccaggaatgagaatagaaatgaaagaagataatgaaagttgaaaggactgggggggcttgacaactgtttagacttgaggagtcagataaaataggaagccaaagataattcagaatattttgattttgattttcatcaccaaataagatagtagtactatgaagaaaaaatggttaaaaaacaataataataaagagaactcctccaaatagtaccaagggagggagtttaatagaggaaattaattccgtaggtgatgagagtcctgagaagccaaacgagaaaagatcaaaacaacccagggattggcagtcgcaggaagctgttctcacttatggctggggctttaagcacaaggtgacatgagatttcagaatttgaagtcgtctggaggcagctaggatcaggtggggcctgtcctgttcggcaggacctgcaaccacaggaggaggatgcgtcaagcagaaagttggaacacaagaggggattcagccataagccacaaaataccttccagagcagagagaaggagaaataccctgaattccgtattttccctgccatttagttccctgctattgccacacattgacgtattccatccagagaagtccattggcatatgagtctgggaaatgtagttcccagggggacatgatcttaagggaaatagacaatgactggtgcaacaactgacctgtgtgaggcaggagggaaaaaacaggaataatatagtttttctctagatcccttcatgcacaaagatgcaaaagaaatgtgttggcttaatgagccattctgggtggccctgtaggtggctgtcctacgaataagatttttagacaaaacagagatgacttcaaatgtcacaagaaaagtatcagacaggaattaatattgacttgatctgtcacaggcgtcaatgatttgcattaagccaacgatcttcattgttaatgtctgggaaattgccagcagcattacgactacttgtgtggattagtgtaacggattcccccactaacattcaggaaatcatgtcaagcacagagtgcctatgtaagagtggttgtgtctattcactacatttcttggactaataacacacttagccttcctgaattgccaacatgtacaaaaccagattggggttttttagttgttcatggaactatcatttattgggtagctcctgtagaagcaagatacagaaactctaattaggaataagacagtccctgtacttcaaagagctctcaggggaggcacacaagtaaacaagcaattattatcatacgttaggataataccgtcatggtgataaccactgagtgatagccaaacacatggaagaggtacccaagtctaacttggggtagtcagagactgctttcaaggatatccgagtaagtgttagctaagacatgatacgtatttctaggagggaaattttcaaggcaaggtggagattgtgcagtgacgcccagagcctggattattttggtgactgctagtatttcagaatgacttcagcaaaagttgtagagaagatagaagacaacaaagtataagcagaggccagataatgaggacctggaacagtggtttgctggtaaatgtttaacaagaggctcttggcggggagagagagtgtctgatttgcagcatttggcaaattttgttgcacaaatgctccagcatagccaatttcaagctaccagtgtgacgtcattgaatgcagaattggaaagaaacgggcagtagcacagcattgtatagttattttcattacccagatataatagataaaatatccagatggtatttaatagatatggatgcaaaatttaaatatatgtacattcatgtgcttcatgttactgaatgcgcacaacattcattatccattcattcacgtgttaatttaacaaacatttctgagcctctgctctgtgccaaacgcagttctagctgctggaattacagcactgaaaaaaaaaatttgtcctcactgaggtaagacaaacattattatgcccattttacagctgagaaattaagacatatgaggattaagcagtatagttaaaatcacacaattggtacatgaaggaatcaaagaggaaatcagctctcagattttaaatccagggactcgtttctgctataccatactacctacctagttgagctggattttatcatggtttccctatttttatcaccatgtggttggataagtaaaataaatatatgtgacctttcaaataaatttgggtcatttttcttggaagctcatctggtgtgaactttaaaatactgcaattaataatgattataataccctggaactctgtagcaacctcttttgaagaactccaaggagcctctaaatgtatcaaactaagttcttcaagtgaattagttatcatctgagagtaatatagacttttaaaaatgcattaattgtattaaccctttcaggcccatagacttaagtgtttctttctccaaataaaaatagtaatctctgtccattttctttagagaataatgaagtaattttcattgaatatgtagtcaacataattacttcaattcaatcgtgaaggattttaaaaattatttatgtctactaacttaaagacatgcatagatttcaagaacttaaaaatgcatattgcctctttgccctatgcctcataaaacaaaattatgataacgttgtgtgttacagaaaaacgcactgattgtaatgaagggtgcttcaaaggccatgaacttggaaagcaacttatttacagagacccccagcaatagcagctaaaagattgactgactccctttattttcagttatccttcagacacttttgacctcttcctgtgcctttctagtcatgtgcaatcttgtggatatctcttccttcctcttgttattttctatttcctctgtttctatttgtttctaaaaataatcatgtttgaatataggattagcttccttcccatctccccattaccaatctctcactataccgctatgttattaatcttcctgagaaatatatcaggttcattacattagttaccagctcaaaacgtatcagtggctttctagtcctcacaggctcaagttaatctgcatattctgactttcatattctgggttcatgcaaacttttcaactttccctcttatacctacttaggaggaccctcaggttccatcatgctcatgtttcaagccagaagttctcctgcctcttcctctatgtagactccacatagactatgatatcctgcttctcttttaatcctccatcttcagctcacagccacactcctctgtgaacagttaaatgattctcccacctcttacctcctatagcacttatttttcatgcagcatttttgagacttaattaaatctacagttttaaaaaatgtttttctaccacagtctcttattcatactaaaactttcaagtctatccattttgcttatacaaccacaccgttaggtcttttaggtccaagaatacaagagaatggcaaagcacgttgtttacatccacacatactgtgtaaattcaggtaattttttttaatcctatgatcctcaattacctcacctgtaaaataggtactactcatactgcagaactcttgttggaattaaataaatgagtgtattaaaaatgctcaacaagatttggcacaaaatcggtactcagtaaatgctaatcattattccctttctcttcaaagctccacaattctgtattcatatcaccctctttatatcatttgcaaaaatgtatcctattccaactctttccacctagcctcaacatttacaaacactcctggtgggaagggaaagcttttgaggagagcacatctatactcatttacttctcagggatgcaagctgccctgcttactgagggcatatgttcatagtcacaccggagcccactgtccccttatactctcaaatgggcagtagcaaatcatcttgatcggtagtaatgacctgtctctaaattttcacatgcatcagataatttcttttttagtaagtgttatcttacatatatgccaaaatatcaccattatatggaacactagctgaaagaaaaattattcagtagtcttaattttctagctaacataaattctctccattttcatcatccatttagattaaagactttactgttagctgaatattcagagactttattctgatttttaaaatttatgaggttcataatgttaagacttcaagggtgagctgtttgtgtcatttataatgcgtgactagacagtaactagaaaatggattgttgactttacaagatttctccccaccacgtccccccaaacctgtgctgctgtgtatttggcctgaaatctttacttctagtcaatctttggacctaaagcctaccagcttttagcatcctttaagattgacgtgtctctgggagaccaatagatgctaaaccaaatttcgtatgcacttggcaatataggataataacaaccatactccctgcaattgtttcctaacacagatgtaacaaattaccacaagctgggtggcttaatagacatttattctctcacaaatctggaagctaggtgtccaaaatcaaggtcaattatccctctgaaggctctggggaagaattcttccttgcctcttccagcttctggtagccccaggtgttccttgatttcaagcagcacaagttcaacatctgctcctgacctcacataaccctcttctttgtgtgtctttctgtgtccactcttttctttattattattattattattattattattattattatactttaagttttagggtacatgtgcacaatgtgcaggttagttacatatgtatgcatgtgccatgctggtgtgctgcacccattagctcatcatttagcattaggtatatctcctaatgctatccctccccccctccccccaccccacaacagtccccagagtgtgatgttcccattcctgtgtccatgtgttctcattgttcaattcccacctgtgagtgagagtatgcagtgtttggttttttgttcttgcgatagtttactgagaatgatgatttccaatttcatccatgtctctacaaagaacatgaactcatcatttttttatggctgcatagtattccatggtgtatatgtgccacattttcttaatccagtctatcattgttggacatttgggttggttccaagtctttgctattgtgaatagtgccgcaaaaggacaccagtctttggatttagagcccaccctaaattcatggtgatgtcattttgaaattcttaactaattacatcttcaaagaccctatttccaaatctggtgacattcaaggtttcagggacatgtgactattcaggggaaactattcatcccaccacatcccccttgaaaattctggaaaatgtagtaataaaggcttctgataaattagtgtggaaagtattcacggttataaattactaaaaagtctcactgtgagctcttaatcaaaaggccctataaaacatttatttgcttgattaaaactacacatccgatattttggttttggatttattattatttttagacttggaataactattttatgtgaaatagattccataactgaagcagcatacctctcaatttcccaacatttattttattattttttgtcttcacactacttaataactgaggaaaaatcatttagaccaaagttcaccttggttgacaccatccagacagctacaggaaataacaatggaaactaaatctctaagaaaaagagtctttcatgtgaaatattgcagagttgattctagatatatagctgttggaagaatggatactattacatagatatggcagagtggtatccagcacctttcaacaaagatctttcagagtcagtcttattatgtctggagaatttacccagggcttaggtgcttttactgacaatctaaccacctgcaccccacccaccgtctaaagctaaagtttattggaagacttaggaaatcagtcttcggaatgtttctgagactggtacacccaccacttcattaaagtgcttcacttcacttcattagacaagaagtaaaatacttgtcaggaaattatttatagtaccatgtatatgggtatcttatttaatactacttaatgatggtactacaagttatataaaatggagaaataagtcatcaagtttgacaataatgatatttgatattatcattatcttttttattcgttcccacagaagtactctgttattggtttagaaaaatgatatttgatataataaagaaggaaaaggtggtaatattctttattttttgtatctttataccccagctctttcaccaatctcccccatctctgtagttctcctctggtgtccccaggcagtgaactattcccagtggttagggaacatctcattgagtaagttacatcaacatttcttcacatttcaggacaacaggaacagtgccaaatcctagcccattgttcaactctcaagccttattatcctaataacacatccatcccaagaaagaattcatcaagatcagagaggaatacgtataattttttatagtacagtatttaaaatgaaacagcttttggcccgcgtggtctcagtgggctcaagggggaaattcaggatgctagctcatctcacaccaagtttaataaagggtgtcctataaaaagctaatttcttgctggtaaattgctttttaagtaatccttgctgttgcaagagacccattcatagcgctgacactgggagccatgttggaaaggctagatatgctctgggagataaggtaagatccaggtggaatcttctctttacagaatgacaatgtatatagctaatattgtcctttgaggctagtttgcatgcagttgctggtatggcactgctcagcagcctgctgcagataagaatgagtgatgatgccctagattttaatggaacttttagagtgcatgcagcagtggggtgcagtcttcagcaaagaaaaacgagctgacttgcaggcatgagagatcatcaagaaagataaagaaataggacatccactctaggttaggcaaggctttttagaggatattatggaaatgagcaagaaccaatttaatttttataatgccactccatttaactttaaaatacaaggtcaaggtactgtgtttttcataatgattaaagatttggagcactctttctgttgaaacatactgcatctgtttggcagaaaaaaaaagtgacaaagaataaaactgggatcagagaacaacaaaaacatattctgtcacttgcctaacacaagttaaaaagcaaaggaaaaagagacaactctgatggacatgttcatccttatcccaacagaaggatttatttacctaaggtcctattatttcaagttactttgatcccaggatggtaacataaaatgtacattttaaaataaaatggaagtataagatcaataaaaaccacatatctgtggataaaacagcagattcaatcttgtggctgaaagtttgctttaacccaacatttggtaaactattcactctgtaatttattaaaagacatactgttattataaaactatctcagtttgcatcttgttggttctgtcaaaatttcatcctgctaattctcaacttgtaatatctctgatatacatgattaatctattttaggaataaaacaaaaactacctttatcttacgcatttctaggaagtgtttttagatgtaaagtaggggtaattgtagtatagtggaaaggattttgaacttgaagccagaacatatgtctctgccaaaaactaggtgtgtgaccttaaataagttacttagcttcctgaatcttagtttgtttagcttttttctataaagtggcacacctatccacatcacagttttgttgtcaaaattaaataaaatactatattagaaagaaacttttagaaagaaatttataaactgaaatgtactatacaagtttaaatcattctcattattttcttaccctaaaattttgaccttatttttcttagcaaatggctgaatctgtaaaatttaacccccacgcagcatctggattcaagagaactacggtcatttctttatacagaatactaattatacacatatagcaaaacacaagttttttccaactactctgtgtttttaaagattcagtgtgggcagaaggaattttatcaactatgttaggggaaaaaagtctgaagaaatgaaaataatgagaaaaagcactgttgatttaagtgcaggaacataaaacttcaaggcaaatgtgaggccaactgagttcatatatatcctcacaaaatgatttagttaatttaaaaacttttctaataagcaacacaggtaatcccaaattctatcttttatagctctaagagtccccataatttattcagcaattatttaccacccacttattataagaaaagccctgggataagtcttgagaagaaactaacaaaaacaaaacttgattgtttgctctcaaaaagctgggtctaaaataggcaaggtaagattttgttttgaggagcccgtattttccagcactgtccattgtaacattaaaatagtttgccaaaatcctcactctgtgggtgtatttgcctagggtgctaaaattgcttaaaaactttgttatttggctaactaaaatcactgaatagtaaacagtagcattagagatggcagagacattaggtgtcatgcagttcaactgcttcacctagcagacaaagacattaagttccatttcttaaatttaactatctggttgaggatacacagtagcagagctaaatcaagaacctcttggggttagagtttttgtttatgcattactttgttttggaattaaaaacagtgcctgtttgctaagttaaattgaaaatatgctctgaaggagaaaaacagctataaaaatagacttaacttccaaactatggatcacaataaactaaagaaataatttctgtagcaataaactccaacactttccataggaccagaaaggcttgagaaagaggagaacaaaaaaatgctttggggcttaccatatatatggagaaagctaaatgaataaaccagttgaaagacagcgagttatactagtaacaatattactgatatcggagctctcacttataaattgtatattatgatcatagtgactaggtactttatatctgctttctcattccttcctcacattaattcacatgtaggacagattacctcttctgtttctatccagaggcctagagctcaggccctcatcgaagacagacagagctatcatccttattctaaaaaaaaactaagaccccagacatagctgtgctacttatagactagaatgtgagagaaaaagacaagctttcatcatgggcttaacaaactgaaacacttcttcaattttgagattgagaaacttagctaatgctaggtgtaaagatgatatgctaccttcataaccttggtgaggagaaattagcatttctctcagtcctagaaggaggatgaccatgaaggtcttcattctcttgagaagataatcaaatgcttcactgccctgttaacggtttactcaatattcaccaagaaaagtagatgggattatttttgcagacacttatacgggtaatttattctgataagcagagacatacctttagtgcataaattgttccctttgtgctctttgtaataaacatcaccatagagaacaaacacgaagtaatgacattgaattaaaagacaccatagaggcaacagcgactggaatttgtgaaagtaaaaggatagtgcaaacagttgtgcgttgcattctgctctgaagattaacaagctgggtcaggctttgaccatcatgatgagcaggagatttttctaatggaaatccccaatcaagttcctgctgcacccagaaaggaacggcttacagaaatcttacatttctttgcacataccaaattgcttggcatattctatcacaaggtttactttccagggaatgtgatcaagaaatcatgatcctaattcctagttaaccctcaaagtttctcagaacagtcagtgcatcactgtcaacttttgtgcaatgtggaaatcagaattggtcacacgtttttccggccactgttttagattcatataatattagtgaaatcatgtcagactggtatagccatgaatttatacttcatgaataggcactcaataaatagtggattaaatcgaccgatttgatttttacctccaataatttcaaaaatatcattgaagacaaggttgttgaagctgtcacttttcttgctgaacctttgttgtgccaggaggaacagatggtaaaatcaaaagtgattagagaatcagtggggtgggggtgagattggaggggagaggtcttcccagtgagacccgctagcgtcttccctgagcagtatgttaacccaagacaattttagaaatctgtgcccctaagttgcttgacatccaaagcacacttgatgcatcctacatttctaaatatttttattgttgtttctcggtagtaatcatctggtttagtcactctaaaagtcaaggatgaaattttaaaatgcaaataaaagtgcctactttctctctttccaattcctttttgttttattgaggtataatttacatgcacaaaaaaatcgcctttttaaagtgtacagtttgatgagttttgacaaacatatgcagtcctacaaccacgtccgtgatcagaataggaaatatttttatcacttcaaaaagtttccttgtactcccgttgcagtcagtctcctgccccaccccagcccctggaaaccactgataggtaaaagcacttttaatctgaaaggtatttaatgtatggcagtgtcagtggtaataataacaagatttattcattggttcactgtatttttgagcacttatatgtgcccgttgtatgcaacccattatgctcaacccctgccctcctcaccagggataaactagtggcagagatagacaaagaagccgtctctctatcacccctatcttatagaacattcttcaatgttagaaatgcagtataatgtggccattgagaacttgaaatgtgcttagtgggaatgaagaactgaagttttaactttatttaatttcaattaatttaaatttatatagccacatgtggctaatgactatcccactggaaagtacagcttctatacaatatgataatatgatacattataacgcaggagtttaaccaagtgctaaagctttactatcaccagggtcactggtgttatgtgaaaagaaaacttacaatagaaaaataaatcctttaaatagtcacagacctgagaaagtttccttctcaagggaacacacattggctcattcaaaggaggttaaaaactagcatttaaggtaatttcatgaagctttcctttggatttctcatgcttattgtatacataaataggcaattttcgatgggacctaataaatcactgttttttatttgaacattttaacaaaattatcaaacagcattgcatttatgttcaacctatttgttctgagaaagacaacgattaagtagaagtcatcaaagttaccagaacaatttttgttcttatgttttagaaggcattgaaggtgtttaaaatgtacacttatagagtcagagtactatgcaactgtggcccttatagtttatccgtcatgcatctaaagccattgttacatctgtttctaattgtgcatggattgtccaagatacacaattggaaattccattttatttatcaatttgaagaggtttcacccatgtggtcactatgatcactatggagtcacattaaattgagaagtctccagaagttgcagtatttatttaaaattctaactttcttcagaggaacaaattctccatttctggattctgaatcctcattagccataaggttgttgtaagaatttgcagctaataggaacacatcctggggagagaccagttgaaaagtaacttggttctgagtgaaattatacagagacagtttctacttcaggtggtgttgctaatgaagctatcatggtaattttagcccatatgatccctaaacgacttcagaaccacttttcatccactaagaacccacttcaaccactgccacgttcactaccacagtataatatggaacaccctctggaattcagtaagtaacttcttaactcattggctatagagctttgcctttgtaaattctttccttttgcagtaaaagagattgtttcaaagtaatccaattagtccctaggcatgtctagaaaggtagagtcaacaacagtaaggtaatagtccttataagatatgtaagaaattatcagtcatttactttaaaataatttgtacacttttccttttatatggttcttctatgttgaagccagtggtcatccagtgattaagattagccaaactcaaaaggctaaaactaaattcaaatggtattattttgctttaattttatgcaatgctatgtatttaaatttcatgaaagtttcgtatggcattgctatcaatttcagtcaggataaatttcccgtgaaataatccacaattttcaactgtacgttgggtacaggtaaggaaacacccttaagagcttatccagttattagctggtattataaatttcaagtaattcaatgttcaattaataaacagttactttaaatgggaaagtatgagtcaagagttagtacaaaggagaatcttaaaagatgaacatcaaagaatcttactattgatttgttggtgcctttgcttgcacttctccaaattgacttgacgttttaaatttgtactgataatcatcagagtcaaatctgcttttaggcaaaaagtatccgctagttattcccctactatgaaagtgatgagatgaattgatcatgtctccagtgtatggatggatgtctttgaggaagacctactgaccttatgtttatcttctgtcagcatggtgtgactatgtggagagacagtgctatttgctaaatactttgtttttcaaataaaaagatttcacagattatgcattgtagaatttataagtattcttttatgtctttgaatgtgccaatacaatttttatgaagttggaactattttatctattttaatgaaattgtaagccttctgtgaattcttttattaattttattctgaagaaaatctgaccaggttagggaaatcaggtcaggttacgacgtgatcccagtggaaaagctgaactgtggactgtgatttaaaatagggaagaggtactgaagtgttgtttttatttttgtttacaaatcagcctttctaactattatgtactcccatccttctatctttttctccaccagaacgtattaacaggcatgcatataattaatgcttttcttgagataatattaaaattaacttcatctgtcaggccgtctgggctaaaagtacacagtcagatctgggtaacatttgagttgatgtaaatatgcccacacatactgacaatgcttaccatttattgtgtgaatgaaaagcagtgtaaatattgtttgttctactagggaagctccacattttaatcaaactttgaccgtatttctaaaatgccagagcatctggaattgttaaaggaactgatagtttttgtgtttttaactgttaggatacttgaaatccaaagggtaaagaaactcagctgatttatacgtttcttcctctttattttaatgtgataaaatgtagtttttgtcatgggctgacaaacagtggtagactacactaactctgcgtttgctgggtttaatcttaccctctcaaggcatggaatgggagctcacttcagacccagccatgcttcactgtccactgccttctcatggatatagtgtgaacattaattagatgaattccataaagtgctttaagctctttggagaaagatactcgctgcataattattcttaactcccatacgctcttatgatataaaccattctgccaggaaatcctttttagggattatcacttaaaatgaaattttcattattaaaagcaggaagaatatacatctactgacagacgaaaatgtgcttaaggcgactgcttttaaataggcagaaatcctgaactatggagccatccatgcctgaaaatactgagtaataatgaaaactggtagcaaatttggaatattaatcatcacattaagttgcaaagaaaaaaaaatacaagccacatgccctttaaaaatacgtgcacaaatctttattctagaaatatataactttaggcctaaaaaagtacaaaaagtaaattattttatggctctgaaagtatccttaatttactcaggtgacaacaattagtgtttaaagagttagttttcaatcttagctacaagttggaattactctggaagctctaaaaaaacaaaaaacaaaaaaaaatagagatgcctagttcccacctgcagaaattctgatttgatttttctggtgcgagacctgagaataggaatttttttaaagcttccctagtgattctagtgtgccacctaggttgccttaaggtaaacctcatattatgcagaacctagcaatcacctatcctgattttatagacgaagatcataagacccaagagggcaaattgatttattcaagattgaatatacaaatgatagaagattcacataagatgcagtatacagagtggcttgtggattcttgccaatgcaggcagcagaattttctttagggttcacccagttcaggcacctctttgcagcagcacttgactaaggttcttctgattggatcattatatgggcaaaaagaaaaagcttaattgaaaagagctgaacccacattgtggaatggaagatatacagtttacacgttataaatgattaatattcatgaaagcatactgccctttcctcttcccttcccatagatgacatcattgcattggtgtagttaggttggtggtttcttgttgttgatcttggttctgacacagttcatcacttattatcctggcttattatctacttctacattcattgttcactcactcactaattaattcaacatggtttttattgttttggaccggttatatgcctgcaacgctacgtaaggctgaggatattacaatgaacaggaaacaaccctgaagtttaaggtatcaagcctttgagttactgtcttttatcatagctgatataaaattgaagccccactttttttgttttcaattactgaaaattcagtgctaaaaaaatgtggatttttattcaactagataaagtactacaattaggtttccactgaccttggctgtttttgttcccagttgccattacataaatctgtgccactcacaacttaggaagggtgtaacattctctgtaatagtttgcctttcgaatagtgtttggattcattactgtccctcgcagtttggaataatgaccactgaataatcagtgtttggagactaaattagtgctgcaaaattccctcaaattacctactgttcttttccctgtcgatgtatcctcatattcactatgattaccctgagaagaaagatattgttgagaaccactttacctactcgaagttttggtatttcaaagattcatacttatgtcatgttgattacattagcactaatactattggcagaattctaattcacgttattttctttttttccaatttctctccatgcctatgtgttgtcccttcgcagctataaagccatggccgattcatgggtgcttttgttaaggcgttcagcagtcacgtttgtagatttttgaatgggacttagagcccttttttgttctttatgtatttctctatttctcagcaaaggaaatgcagacatgcaagaaatagtgatcaaatgtcctgtgtactattgtgggtgtcattaatggtatagggagaaatagaaaatagttgcaaagatgcatttaacaaataaacgaggtcttgagattcaccatgaatgtggccccttctatgaaaagtagttaacatccaactgcaaagttgtactggatcagtttgactttaacctttagctaatatgaaaatatggaattgtgtggtggtgctcacaaaaaagaaaactcatttttcttaattatcatcaattaacatgtactgactacccatgagggaaagttaatttgctcttgagtggaaccagttatttgccctattatttctcccttgcttattcccctctccctccctcctccctttccattcaacaaagaaaaatagataaagcaatttctgattagccagtgaaagcctctaacataaaatttccaaagatgtgccataaattatccacaaaatgtaaaacttttcaattttggtttgcattttcttttttcttattataaaggtaataagtgctcattatagaatttgaaaaatataggaagttgcacggaagacgaataaaatcagccataatcctacaaacctattgacacttgtacatatgtttgttatctctaatgcattcattatgataatgcatcttttcaaccaatagagtaatcactggtgactttcaaatttgcctactcatttttcactctgtggacttactttactacctcttgccctttttcagtaaatgaataaatatttaagtaagtaaatacaaatgtaataacttatgcgctcaagcacacagatacacacagagagaatttggaacttcggaaatgccatcctctccctagggccgcaagtgagttgataagcacgtaaggaaggataatcaggggagccttctcgtattgcccagatggctcaaaattcgtcatctctaccaaacaactatttggagctttgaagaaatatccatgacccctttgaattcttcagtttctttcgcgttcactttgagaaccaagtgacaagtgaatttcctgacttggtcttttaaacctgttagcgcagttccattgagattttgtgggcacaagattgcaatgaagagatcaacagggagaaattcatttccctatatatgtgcgattaatccggagtgctaagggcagatataaagcaggtgcctactcctgtataacttggaataaaaccatttccaaaggctgatgatcctcaagtcttgttctgcaaatgactgatgtataacttcaggccaatttttctccagttagtctgtgtcactgggagtcccatttctcggggagcagccccatgctttgtcaggtgcggagcccacagaaggttaatgcgaaaagaaggcctcttgccagactgttttccagatgatacgtagggttattagtttgagctccttaagaagatttttctcacctgtcctaccaacttatgtttatttcattggtgttagagggtttcagtggcggaagtaaaatatttagcggggaagggacagcgttcatgggaattttgcctaacttaattttgtatctttagctcattcgtagtcattgtactttgtgttttgtcaactgaattttgtttgcatacaaaggcacaaaatgtttgcttcagacctgtcactcttatttttagcatggttagacaaaaactgagatgctttaattgtctaacttatcccagtttaagtgctgcaaaatctcccaggcaatgtcatgggcaactaagggataaaatcagagatttaaaggtgccaggtttcccacgcttctaacagttggcgttttgggtgtatacaatccctcagctttcttctttagtttatggagtcttgtggagggaatagcaggtttttagctaaaattatcatgctgtcgagttgggtctctagtgcatcctgaagagcttgcattatttacagaggctgggctatcattttaaatcctgatgcttcaatgcccgttatcattcttgacaaactcttccagcccgtggtctgttttcctctgtttgcttccatttactttcctgagcaaccagctgagcaaagatttacataacttttgtttaaacaaaccctgtacagttcactctttcagccagtatgtaaacacttttgagacacagttacatttttctattttagtcccagattctgtttatttgctacattttttgtgcccacatttttgtctttgttaagtctcttacagattcacatgaaaaaccagaaaccgtggctgctcaaaagtcattaataatgagatttttagctactgtttctgcttgtaaattcttcatttcacataatacagtctcaaaaggccacagagaattcagcctcgcttatctctgtgttgcagatgatggcttctagccttacccaatcccagtgcagcttgcttgccatccaggagtcgaatttgtttccatctgacattagcgtattaaaaagattggagatcaacaagcaacaatgttcttgtagaaaggtaatcaaggtttagagcctgtgtgtcatgagactcctagcatttgaaaccgctaaggggttgaccaccattgtcccaagcacctgtttaagattctttcctatgataagggacctaaagtgattagcatactgataagattttcctagaataacctatttatttcagtattattctttcaaatcttaattaccatcttttcctttacccagggtcttctttctacctctacgacacatttaattacctatattccccaacctgtaccatattaaattttgaatggaagttttatagggtaatttattggaaggatggccttgagtgtcattatgttcaatgaatgccctattttgacaaagagatgactaaatgttattgaaatctttttaatccaccacgcttctgcttagatgtaaatgcaaatctgttctttacatttgtgattgaattgaacttgaaaagtaccgccatattgattccttctgcaaataaaatataattacatttccctaaactttctacactctcccaagagattggctggctttgtattgtagatttttggtgatcacagaggacaatgcattatcataagaccaataagatttatttttaccttggtaaagaattttaatttatttctagtttcattttcatttatatccatctcttctcaccctctgctctacaaaagtatatatgactatataaattgaaaaaaatatcaagtgcaaaattacagaaataaataattaggttattttagtggaggaaggtttgttgtgggtggaggaggagaggagtgagccaagaaaaacgagggaccatacgtgatcatatttttgcagctattttaaattgtttgtgtatatactttaaaatattataaaataaaattttaagtgcaatgcatatttggagccaatgatgagggataacttcagaaacgtagcatcatcatctagtgctttcatagtcctttcaacatttccagatagttttaatggcctgctcatggaggcaatgccctaattttaacatatctcttcacaactctgatttcttgcttcctaacattaaatgtcttcaaagcttctttcaccactaattccttatcaagaggataagccagtttattctttaagaaaaactagctacacaaaaccgtaagtcattccaacataaatccttcactatcctctctctatagatttggttttgattcctcctgctgaaattcaaccttctttcttcagctatccacacgtcttaccctctaacttccctcaggagtgtctattagctcccattacagtgaccacagtaatatagtaatcccctgctgttctcactctccacttccttacactgcgttttaagtctcttcatattctttatcaccttgtatcatgcatcggttttcttagttgtttattttatgttgccttcataaattccatgagagctcactgccgtatctttagaacatggaacagtgcttggaacataatgggcattccttaaatagctgtagaataaactttcaaaatcaacaataatgtatttgccaaatccattggcttctctgccattttatcttgttcaataccactgcgatattccccttcctttttttttttttttaaagtctgtaaccctttagcttctgtaatattcctagttttttattcctctcatgtgtcaaaatcatcagttgaggcttattgttttctctttctcactctgacctcacctttgtttacatctcatcttctggctttggctatcctgttttttatctctgttccaacctgtatttctagccctactacctggacatgacatgtggatatctccgtatgaccgcagtttccatatgactttgcaaattcatccctgctctcccctccaaagtcatccccacaattgacttcctgttccttccaacctattaaggttcaaacccacttttgctcctcctttgcaggctacacttttccttctcagtacctcttttttttccaagttcttagataaaagtcatagtaccttacgttgtaattgccactggtctggtctttctgcctgctttcctttccatttgtaatcacattatccattccaatccatttataatactgtgatcagccataaaaataacatttatcatatcgtttgtctccttaaaacctgtagtagatcccctctatttacaagatctggtataaaatcacccttcctgatattcaatgcctgttttaatataatctcaatattatgcgtcataaatccccctgtgttcttgcactttttatttcttatacatctcatcaaccatgtcttatcaactctcaaaacctgtattggttttcaggaaaactcataaattattcttttgtagaccttttgtttgtcatctttgaagatctctctctgaactacaatattttgtctgtataatcaatttggaaattcatcaggtattgaaatatgacatgtcttctattgtcttgaacattaattaaaactttatttgactttttatatgcttacatcttgtttcctcacggagtgttaacctactagaaagtaatagtttaatcttatatttattttaattcagatttagtagcatactttacacgtggtaggatgtgtaactgccttacaccttgcttacgtgagttattaatgttttcgtatatttaatctgaggatgtactagcaatgttaaaactgtaccgcatgaaattgagtaattgaactatttgttttaaatgtgttgcttaacttattgtaccattttctcataatcacagctcaagttaactttgtggttgtacgtattatttcttgtgaaatgccaacaaacttagagcaaggaaaataacaggtataatcatactataaaggcaaccttaacactagcatagtctcttagctcatatggtaactacaataatgtacagtgacaaagagaatattgtactttcttagcacacactttcctactactctactgttgtggataaaaacagacatactttaggagaaactatgttatttccaaataatgccttaaaggttactccaggaaaaggcatttacataaactatctaggaaaagaaccttttaaataatataaagagctcacccaaaaggactgaagtgtttagttgaaaaaaagtaaaaatgtcgaagactttgaaaaatagtttcttgcagtatattttcatcgcttccacttacgttatgaagacattaagcgctagtttatcaaaaactatttttgtacatgtcttctaatgacagaacaatgtcaacatgattttcatcattgagaatgcgtaaagaaaccctttgtacagttttttctatgaatgttcccctaagattaaagcaaatttccaacacgaattaggcactccgaaaggaggaggggagggaggggagcaagtgctgcaaaacttcctgttgggtactatgttcactatctgggtgatggaatcaacagaagcccaaacctcagcatcacgcagtatacccttgtaacaaaccaacacatgtacccctgagtctacattaaaaatagagattaaaaaaaggaaatcagtatataatctaataaatacctctcaagctttctcatttttaaaataaaattttagattattattttaggaataaaataggctcttcattgtatataagttcatttctgagttgcaaaaatcctctctttatgtttttttccccgtattagcatgtttttctcctgtttttccccactcaacttggctgccacaatcagaaagcacaaagacaattttttcttgcgcttgtaaatcaaaaccttagcatcagacaaaataactgctccaggtctgtcaaatagattcatttgagctttcttcatgcattgaatacggcagaatttctgacctgaagaaatctagccttttccaaatttgctttaagaacattttgcaataaatttaatataataaaaggaaaaaacacatcaggctagaatttggaaccgattgttattaaaaatctcaagtctatcaatttaacttcaacaaattacttaatttctgtgatggttaatttcatgtgtcaacttggctgggccgcagggtaccgagacatttggtcaaacattattctgggtgtgtttatgaggctgtttctggagagattcacatttgaatcagtagagggagcaaagccgattgttctcccttgtgtgggtgggtctgatccaatcaattgaggacctaagtccaatcgattgaagacctaatcaaaaagcctgattaaaaggaactcctgcctgatagctaaagctggaacacccatcttttcctgcctttgagcttgaattgaaaccttgggtcttcttgagtcttaagcctccagttctggggctggaacttaacgtcattggctttcttggttctcatgcctttggactcagacaggaactacatcattggctttcctgggtctccagcttgctgactgtaaatcttgggacttctccagattcgtaatgagccaatttattacaataagtctctccctctctggtttcgagagagagagagagagagacagagagagaaatgagagcacaagaacgtgagtgtgagagtgccctaatataatttctctaaatatcactggttactcttcaaagttataaaattggtataaaaggtgacctcaatttttcatggagttaatgtatgaaagtcacaattaaaaaggaagaattagttctggtgtcctgaaagttatttgaataaattaatatgctatggaggctttaaaatactatgaaaatttaatattgtattattcttagtgttgctatttttaaatagcactttttcttttcctttttttttttttttttttttttttttgagatggagtctcactctgttgcccaggctggagtgcagtggcatgatctcggctcactgcaagctccactgcccgggttcacgccattctcctgcctcagcctcccaagtagctgggactacaggcggccgccaccacgtccgggtaattttttgtatttttttagtagagacggagtttcaccgtgttagccaggttgttctcgatctcctgacctcatgatccacccaccttggcctcccaaagtgctgggattacaggcatgagccaccatgcccggcttaaatagcactttttcttgtgagtcactttttaaatatttgtgcaaaccttgttgccattctactcaagctaatatcctaaaccgaggacattataacatttcaggagtcaaaacttcagacacttaacatagtatcctcaggttcatccatgttgtcataaatgacaggattttattcttttatatgactcaataatatcccattgcatatatatgcaatattttctttattcatccattattaaacacttaagttgattctatatcttggctattgtgaataatgctgcaataaacatgggaatgcagatatctctatgacatactgattttatttgctttgtctctgtccccagtagtggaattgctgtatcgtatggtagttctatttttaagttttcgaggaacctccataccgtcctccataatggatgtactcatttacattcccaccaacagtgcataagggttcccttttctccatattcttgccaacactttttatcttttgtattttgataatagccattctaactggaatgagatgatatctcattgtggttttgatttgcattttcctgatagtgatgttgaacattttttcatatgttgtattaactaagccaaacacagaaagacaaatgcagcttgttctcattcatatgcacaatctaaaaacatcgatctcatagaagcagtaaatggacggtggtcaccaaagaatgggggaagtaggggaaaagcgagaatggggagaggattgtcaatgggtacaaagtcacgattagaaaggaagaattagttctggtgtcctgttgcatagtatggagactattgtcaacagtaaggtattgcgtatctcaaaacggctagaagagagggttttgaaggtttctaccccaaataaatggtaaatgtttgaggtgatatgctaattttcttgatttgatcaagtaaaggtcttaattgtttggcaattaagactcatgaatacaaataaaggtcttaattatttggcaaagcatgctgagttttgtaaacaattcagtagtgatttttgagaataggtcaatagcaaatattaattaaaatgtcttctatttatgacctacagctagatggtaaacagatagatgatagatagataactgatagataactaatagatgacagataaatgataaatagataaatatagataatcgagagagaatacctttcccttcacacacgtgcatataggcacactccatttctatcatagttaccaggattcagacattttgtctcactatttttctcaatgtgaacatgcatataggaatattatagtttttgttctgtgcccattttagttcgttttttaatatttcaggacaaaggcaatatggcggtttcactttgtttttcatttttgcttatactttttaaagctcagtgtagaaaagtttgaaaatacacaaaagtattaaattaagacagctgggcacagtggctcacgcctgtaatcccagcacttcgggaggccaaggtgggtggatcacgaggtcaagagatcgacaccatcctggccaacatggtgaatcccgtctctactaaaaatacaaaaattagctgagcatggtggtgtgtgcctgtagtcccagctactcgggaggctgaggcaggagaatcgcttgaacccgggaggcagaggttgcagtgagccgggatcacaccactgtattccagcctggtgacagagcgagactctgtctcagaaaaaaaacaaaacaaacaaacaaaaaagcacctatagtctttctcccataggttgccttcttaatgggttttacaccttttgatgttttcttgagttctgtcccattagcaagtagtattgtacaaaaaaaattttatcatcttttatttaatattttattgatgtttaataattagaattattttaaattttatatgtcattttaaaatgcaatacaatatagtaaactcccagatgtgattgtaaataattaattattctcccattattgggcattgggactgcttccacattttggtcactgcagtgaacatccttgtacatgaatctgtatgttgaagttgatttcattccacactccccttcattcaaggggctccaaccattctcgttttctttcagcttctttatatccaggcatataaagttccttcctgactcgggagcgtcatacatgctgttttctccatctggataagtagttaattctgttcttctttgtgcatctcccgtttcagtaacttcatctccaaagcctttccaggtcactttatctaaagttacaccataatcttgcaaatcctcaactattgagcattattagtctccgttatcattattctccattattctctgtgaaagcatcccgtgattttcttttgtccctattaccacaatatgtgtttattccgtgtatgtacatctttgtttgtttattgtttgtctatacctgcaatgaaatgcctaaggtcaggaactgtctgatgcaggatgcaatgcgctcaataaatatttactgaacaaattaattcatttgctcagtcttgcaggcaaatggtacttctgtatatttaaatatctaaaatgaaagcgttactcgttactgttggttgtcaatcaaaatttaaatgtcgatgtttaagcgtgaaagacctctgtcaagttaatctgtacttacccaaaggctattatgtagaagcgacataaatattttcctaaatgttgattttcatattttaagaagacaatgaatgtttcaaagcattttcttctacacagctatttattctggagagtggggcatatgtttcttaatattgttaaaattggcaaggggatactgttgctatatacaaagaacacctaatcatcatgcagacgttttgtttctggctctcagttatgaaaagcagagattttaaaaagttacctttatatgctaaattaggaatggcagaaggtaatattctaatgtttataagtggttcttctctgagtccttggtttctatgtttatgaattctctttttgaaagaaattatagttattattaccaggtctattcttttacattgtttctaattctatggtgatcttcaaaatagagtatcaattttaaatacttgggaatgaaattattcttcccatatcatttctttgtatggcatacattgtgatttgttgtcccatcattgtttcagtatgacctgttactgcaaaaacatattgagataaatcatcccacatactctcggccaggacagacatcacactgttgcagcaacacttcagatgagccccattcaaccttgtgtttttatagagaaggatgccacatgtttatattcatttctgaagattggctcatattatttattgaaacatactagtttaaaaatctgtccatttatataacacctggtctatctacataacttgaattacataaatataaaactaaacttcccctcttctccagtgtatagcttgcaagcaagtgcatgtgaaataaattaaagccttgtttgtgtttttttcatcatgtgagtacaagacttttcaataaaaatgaattacttttgaacatatttgtttggacaacaaacaagagaaaagatctatttgattgatagtggacagaattttcattaagttcaacagcagaaataccacaattgcatcattcaccttcgtgtatcaaaagaaaacagaaaattagatgtgatgaactctacacaaatgttcactatgcatactttacccattaaatacattatcaagaatcatgtcagcatgacattctaatatagcagctttacaaaaacatgtaatctaatctagggatgctgttgtcctctttaaatcagcttcaaacatattctgggttgatatttctcattcttttttgatccacattgtttattcacataatgattatatttaactgaagataacagcattatcaaagtgaaagacaaaatagatgtttaataggaaagtgagtatcgaatcatcttttttctaccaaaaacatctataattatgaagtatttggttaattattttcacaataatttaaaagtgtacaacttgccgatttttttgtactttctacttttcatgtctcgcatatatctctttaatatctaagtatttgagtcagaaaagagccagtaccgaataatgggaatctcactgaaatgtgataacaatctggggcctggtcctgggacctttatctgcaggacaacttggacaaatatttagacccccaattcctcgtctttaccctaggaataataacacatttttctgacctcatacttcacgtggatctcaaatggaacaatcatctgatagcactttatgaagtatatgaaagcaataaattatcacaataagataattgcaattattctttggcatagtattagtgatgtctttatctgtctgacaaaatcaacatttctgtatggtaactgcctttccttgttttaacagaagatcatgccagaaaagatgagtaggtagatacttaacttgttgttcctgaatctggaatgtattgcagatgtcccagactgatctttgttcttttttttccttacaaatttcttttcacattgacagtgtgatatttctttaaatgtgcaatacatagctaaccttatttgtttgtgtttactaattaaaatatctaaactgcttaaaggagaaaattcagttttaagttttattgatttatacccttcttcaatccacataggattagggtagtatgtaacaaaatttcaaactataaatgaaatattgagttttgtattaaggccaaggatgaggaaaaaaaaagtaagtatatatggaaaaagaatggtattgaatgggagttttgatggagcatgttgacatcatgataatacctattatctttatattctgaatgtcagaacaaaattagagcaattttcccttatttccctacaatacgtctgtcttaataattctaagctttcctgatttcagtagtaatctgtattttgcaaaaggcagcatgtttataagatatcaagtaaactaagtttatggaacttgtaacagcatttttaacaacatttctccctagatagttcatggtagacatgaatttattcaaaactagtatgtagaaaaataccattaacaaaagctctgaaattatattagaggagctgaataatgttacttgagaaagaataaaatgttatttatgatttttggtatcttttacccactatatatggccatatctctgaaaaactttagtaatatgtactaatgcaaatatggtagtaaattatgtctacaggtgctgataccatagtagataaagtatgataactttattttaaaatatcatatttaaataattaatatacagtactgggaaagactattttatctattctctcactcttgaataaaaaaatccagaaaaaaataccttgttttggtaagattatatcaatttatttcccaaatgggtagagggttatttttttctgatcataaacgtatgtctcttcattataaaaatccactaaaagtgatagaagaaaaccaaaagaataaatgtaaacaatgatgccattttccaaaaatcaccttcgacatttttctggatattgatacagtctaaatctcttttcggaagactccctcctgtgtaggttccccaactactctgcaatcttatttcctcttgttctgttcttgtagaaaggagacccattgtcaccatgtcaaataacacaaaatggtgcacgtataagatcattgtctctgtccattatttgccagaggacctcaaactttttcaggtggtgggcaactggatgtcatgctgctccttgtacaacagaacacaattcattatttatatggttatttcattttaagaaaatttaactttcattagctggaaaaaaaaagaagtggtttttaagttgtttagaaatgtgaaattcaattttcatactgcaaaagagattcaactgcaaacacaggcacacatgtctggtgtaagaacgagttgtcatacaaacccaaattagctgcctccacgttgtctttgttaacaagtgtttgtttgctccttgttccatcattcagaaatgctctttagcaggaattgatggaacacagtcgcagtgacctcttcctgtctttaaaaatcgagatgacatttgcccatctgcagtgttaacatagttcctcaaagaccactgacagtggggtaggactgtattgcgcaagttctctcatttccctagaatataattggtccagggccagagattttagctcatttagagcagcaaggtgctcttttaaaattccctcacctattttgggcttcatttcccttatacggttatgccttttccagtctgatgaacattctccttgacagagcagacaagcaaaaggagctgcacactgctgctttctgtgtcgtctctatccctaaccttctcccttctgccccaatcagtgaaccttcgtctttctggttcttcttcctccaaatggaagtaaaaaggccctgaatgttgtctttaccattatcacgagcctcaattcattccaagctcagcttttcctcactgtttatacagttctatattgttcttctaatatttgccctcagttctctgtccctcgtttcttcccatgttcatactctattagaatctgagcacctttgaggttgtccatacagtggcacacatctttgttttatactcactgggatgatttgccattatattgtcaaaattttattctaaagagcttttacaggctttcttgagccattttctcttgaaattcaagatcgttgaatctctacgctttttccttcttaatctaataaacatacacccccacatacacacgtgtgttcctgaaagacagatgccacttgactcgtcttatagattgtctaaattgatcattgtgtgtggggataaaagggtgaattgtataatatccctgatggttcacgaagtctgttcctgtataacctgattagtcttctgaactcttttaaattctgtctgcaaatgactgaggtttggcaatcagcctatttcagttagttgttttcttgcataagaagggtccatatgtactgtgtgaagtaagagagagaaagtacttagatttgctggatgccctgattgttagcatggctaaggtattgtgtaagtaaggagagcagttaaaaatgatattgtttttatttcttaattgaggtaaaattttatataagatgaaacagacttatttgggagaggaggaagagtttgttcttacataacatttcaacctgtcatatttagttgagaacttcaatctgtcaagatactttgtataatattcagattctgccatctaatatattttccacgctttcttactgggtgtgacagtaacttatactgtggcaggtgtataagttagtaaagatattaaatgctcaatctgttaacttttgtgaagtggtcccactgataaagtgacacctcaataaaataaaaatttccattacctcagaaagctttttcatgctaccttccagtcaattcccagccccaataggcacctattcttctgatttatatcaccatagattagttttgtctttttaaaaatttgtataaatgaaatcatacaaaatgtactattttgatcagcatactacttttgagattcatccatgtaagtgtatcagctgttcattcctttattgatgattaatattctattgtatagatataccacaatttatttatctattctccttttgatggacattcaggtggttttcagtttttggctgttatgaataagatgctgtggacatttgtgtacaagccatttgtgagcatatgttttcatttagtttgagtaactctgtagaagtggaatggctgggtgaaatgtttaaatttatgagatattgtcaaacagcacctaaacagttttctaaagtggttgtgccattttgcaatgccaccagtgatgatggagagttccagttactctacatctttgtcaatatttggtcttgtcagtcattttaatttttgctatcttacagaatatgtaggtatattgttgtggttttaacttatattcctctgattactagcactattaagcatcttttcatggatttattggacattcatatagattatgtgtgttgaagattattacctttatgattattgggtgaaaatagtatcattttgaggtcattcatataacttgaagactgggaatgacagacattttcctgttttgtttcttttctttttactttatctgaagagtctactagaatgcagtgttgctgcctgagcagcagggcattagctttgtaaaagctctgttccttggcaaccccaccactaatatgaagtgcagaacatttgaattgtctttgaccagcttcagcatcagcactattttttttttttgctagacccctagtaggtatttaaaagtacagaaatagaatttaatcatgctttttaccaaatgtgctatgctcttagagattctttcaacgtgcataaaaattctgcagtttcaccacataccagtaaaagaaactcagtcactcatttagccatttagtaaaaagaacaaattaactgatgagcatagtggagacctcaaaggtaaagaagacaatgtccctgaaataaagacaatcataaattttcaatcaaaataatgaaatttaggctgggcatggtggctcatgcctatgatcctagcactttggaaggctaaggtgggaggattgtttgaggccaggagttcaagaccagcctcagcaaaaaagtgagaccctgtctccacaaaaaaattttaaaaattatctgggtgtggtggtatgcaccggtggtctcagctactcaagaggctgaggtggaggatcaccagagctcaggggttggagactacagtgagctatgattgtaccactgcactcaaacttgcatgacagaatgagtccttgtctctaataataacaaaatttaatttttatagactgtgaaaaaccattatgtagatacagttcaagtacagtatgattttataggatagataacttttgcttgaaaatgtattcccaatttataggatagataacttttgcttgaaaatgtattcacaatagagttagtatttggggcacacctttatccatttaacaaacatgttttgagcactgccaggtagcaacacgttactaggcactagagtgagaaaagattacagttcctgctctcatggatctcatggtctagtcaactggaatgaaaggattacataagtagaggtaaagacacacatgatggaggatggagaatagtcaaaggtctggagaatgaccaggacgtcactgtgagttgtctaattgcactgaagcatggatgaagaattggaaagtcattgtaagaagcctaaaaaggtatctctcagggatgctatgaggttctgaatgttatgtacgctatttgggcttcaacaggcaggcactgagtattcagtataaatttttgagcagggaatccaccagaagaactatgcatctggaggattaatctggaaagattgtgtagaatgttatgcagtgaaagagtctgagatgaaacagttaggagggtgtattaataacataggtgaagtgtaatgaataaccaggctggaggaaaagcaataacgatggaatcaaccgggcaagaagtataacaattaggatcagtaaaatagaatttggattggaggaatgaaaaaaaaagggacaaaacaaagttgaactgctggtatccatactggaaaatacagatgtcattcaaataaataatgtaatgaatataagaaaccagttttaggagtgaagtggatgttggcttgaaaatatttcctttgaggtttcagtcaaatgaaaaggtcctgaaatgctacgtggtagcctaagaaggaagcgttcctagagagaaaaaaattagaaaagatttacatttgataatttaatcttttccttcatacaagctaaattgataagaaagtaaaacctatagttttcaccactcttttacaaatatccctaaccttttagatattcacatgaataattgagaaaaatctaacagatgacttgcttatgtcatttgtctgctttatccttaggttcctctggcttatatattgttcaataaaatacagatcattgatattgtacaatgtactgataatggggagtgaatccatgcttgtgcattcttttttttttttttttttgatttgcagagggcgtgcccagtcaacaagagaggcacaattgtttttatcatcacctcttctcatctaattccatgaaggagagtagtattaccatacaacagataatgagttggaaaacaagaaacctaacctcagaacttaaggcttggggaaaaataaaagagtaatttgtgtttaatgcctgtataacttggcaagagggacatataaggcttagtgatgcccaacatgtgcttagatgtggattgttagttgatgtcttgggggttctgtaatctaagctaaatgctcaaaatcaattaattgatgttagacacagagatctgctttgatccctctttatcgtatttctaggccttcccattctcaagagcctgagaaacgacagctttccttaataacttgttatttgtggtaggagatgaaactttgataaaaacacaattatttttaaatgtctctttttcactctaggctgttgtatgtatttcaaaaagttacttttgaccctttccagaatgagaaagcaatcaagaagattataatatcttgcttagttttctgctcaatttatcaacaaatatttcttaagcaattattaagctgagcagtgctcagcgctgtacttggtgatataggaaatggggaaaagactgtctttaaggcctttataatagtaattacctcaacttgtctgtttcttttccttaccatttcgccaaattcattgatctatcttgttctcaaagcaatcgccatagttatattgtaacacagcattttctagggtgtccccattaagttgagagtgttgacaagaaaatacaagcttatttatcattgtaaaacttgagacacctagtagttaccctaaattaaatatttgttggagtcagtcacactaaagagaacacttactgcattgaacaatttacctacattagacagcatttaaagactatgccacagcaaaggcccatggaattcttgtgaacacagaatagaagtgtattaaggaacaagcttaattctgttctcttaaagcacaacactttctcaaaacatattttgaaatcacctttgaccattttttttaactaataggtgggtgggagttagggtaggaaaacacaagcagcttcatcaaaacgatattctattttcttcaaatttgtggggaatcatacggcctctcaattttctacattatgctaattatgatattaatctctctgccagcaaatgaaaataatacatattagatgtagcaaatgtcaataatgacaaaattagtcatcatgcagatactcagggattcccaaaatatgtttggattatgattgctagctttgagtttgcccagaatcgtttcaataaaaataagggactcaaacacatttggagcaaaactcacatcataaattttagacatagctctgccaataatgctctcagttatattttcagtcctaatatttcctctgagttccagaccagtatcttcaactgtctgattgatactctctccttcatttctgtctccaatgcattaagtcctgtgtatttactttccaaatgccacttggttccatgcacttctctccatttctgccactgactcctcctcaatccaagcgaccatctttcctcactttaactaccatgatatctcctgcttggtctccttacttctattcccgggctcctccaatccattcatcctccagcagagaatgatgactagcaccttccacagtgtctggctaataggaggtatccaatcaataattgacttacagagtgaaaatataggcatggcaaataccagtagagaactacagggttttagaaccaatgacattagatacttccatcaaatatttacagtgtataatcaagttgacttgcacattgtcttatttttgaaaaacaattttgttggctttttctatatgcacacatacatattgtatcaccctctacccgccaaatggcttttgaagaagtatttatgtggctccaaattgataatacctctagagagaagagaaattagaaattttaaaatgacctatgcttcctttcgaatatcacgtcctgagacagtgttttttgagttacgtgcaatatgttccacgatgaaacatttaatgtgttcagaggcatgctagtaatcatgtagaaagaattttatgcctgaagtcacatgttctataaccaggatcacttaataagaaaacaagtacagctgtggacaagatgcctttttatcagggaaaggccaatttgttttctttgcaaatctaagtaaatggagagaaaaacacagcccttaaatgttttctatttgtcctgaagttctcatgaatgagttagaaggcgagaaggattaaataaatccttgaacgtagagagagctaacatttattttagcaaactaaaacctattcgctttgcaaagttctgttctgtactttgtaacaacagttttctttaaaacaagagccaccaattcaaatgcctttacagaatgattgaatgctttcatgccccacctaaaggcattcaaatcattaatcaaacaaagttctaacgccaaaacatgtctgggaccagatttaaaatgtagccctcagtttcagagggcaaaaacttaacatatttatattttcctcactttaggtaacactgtattgaatctctgcttgaaattgaggagcacgtgattttttctttttggcccagggcagcatttcttggaagagaaagaaaaacaacccaagatacccttacaaaacatgtagtacttaaagctctttatgatgaattaattttggtatacacattaatagcagtgataataacaaatctatatatatatatataattgatatgaataagataaatacatcaaaaggaaatttcattacaatttgatattaggtaaatgtcccattaaaataaattgctactgtacataattttccttcagttcattggcaggatgtttgctttggaaaataaacagtctatttctagttttagaaggaattctcattattcttttatagcaaccattatcaggagcagatgggaaattgtaccaagagcatatctactattatacctcacaggaaaaagagagtattaaatgaaatctaacaaggcctgctcctgactctagttcctgtaacaaatgaacacacacatttgtatggtttcagcatttgtattagtaaggtacaataaatgtttactgaaattgaaaaaaaaaaagataacaggagaaagaagaggctaaaaaggtgcattttatttctgatcgttcattgtaaagactgctcctttttaaaataatcaaattttattttatatacagagggtacatgtacaggcttgtcacaggggaatagcgcatgatgctgaggtttggggtacagatctcatcacccaaacagtgagcatagtacctacctgatgagtagtttttcaaccaatgcgcaccctccctccttcccacatctactagtccgcggtatctgttgttcgcatatttacgtccatatatgctctatgtttagctcccacttataagtgagaacatatagtgtttgtttttcctgttcctgcgttaatttgcttatgattatggcctccaactgcatccgtgcttccgcaaaggacatgatttcattctttttatgactatgtagtatttcatggtgtatatgtaccacattttctttatccaatctaccattgtttcacaactagatggattccatgtctttgctattgtgaatagcacaagacaggacctttttatttgactgagttccttgcaaattactaataaaagatctggaggtccttagttaaaagttgaatctgtagtgccgttcaaatttagagatgtattttctgttcaagagaagaaagccctcattcggtcatgcttaatattcagctgtaaagtccaaaacatatgagaatgacacaaatggaaacattttataaatacctatacaaaggaggggcacttagttcccctaggcctcttaaaagtcctctagaaagagggtacttttatgctaactattaaagatgagtaacgaatttgtcctatacaacttaacagtatcgtcaaggaagtagaaagttactcagttttactgggcattggagctaagcttgaaagtgaggaggagaagcggcaggagacggagccgagaaggcagtggggagaagaggaggatggtcctttccatgctccctgttgtactaacatgtttggatattatcttatacttcatatatggactggattcttgtccttctcattctgagctctccttgaccttgattcttacctcctataactttcattctttctttactcaaaaaaaggccatttatttcagccatttttcactgttttcttatccttcctagttgcttttctatactatttttccactcttttttttttctatactattttgcccttctctccattttcctaactgctagatttccccaattttagccatctttcaattgttctgactatcctcaggtgctcccacaaggttatcagaccttccaccaagacggaatccctcagtctatggacaggctaagttgaatgggtcctggtgctgtgcttagcatatgccttgagtatttgtgcatttattttgcttctttacaaaaatccatcatccgatagaagttgaaagaaacttgctgaagcacattaaaatctctgaaaacagtattggctatattttctaataattagcatgactggttaacttgctttatttatcattgaaaaaagtatcagaaactgtatatcaaactcctgaattcttggcactgacgaagagacacaatgagaatgaccttaggataaaaaaacaagataaagcaccatatttgtaggaaattgcaccataaaagtctgtttcacaactctcccaaatttcattttattacatcttttctcttgaccaatcagtaaactcggttaatgatttacctgtctcaaaataattcatgaacaaaattacaagtaaatctcagtattggattcttgaaacatctccttgttcaatgaagtttcctttttcttccctctatttccctgtatttatcttttcttccagttgcattttatctcttctgtttttttatcttgctccctagtttgtgattttttgccaattttttatttcctacataattcatccaatctgtcattgtacaatttcttataactgcttcttagcttattccttttcttcatttgtcacattctatttttcatctattgtgttttcatgcagttttggaaagttttacaaatagacttttaaaaaaatgtacgtaatgttttcatagaaaaggtagtggtttctttttcttatatccttccctgtataaaaataaaaatgtagcagttctttctttgcctatgtttcctctttccttcccccaatttgaccagacttgaaggacttagatatgtaacagtgttattttctataatttaggaacagcttttgacttaaaaagcagaagagaagttgaaaataatatagtaattctacatgtccttcctgcttcccaactctctgcacatgtttgtaacctcccctttcttttttagtgtatctctttcatatacctttgtccccagaaattctgattcagtagacttagaatggaattctgggcttttatattttgaaaagctccccacgggagttagatatgcacttcttattaagaatgaatgcttaatattggaatcaaaacacaataagctttctaactatgatgaataatccaacagatttaattatgattttctttttgtccagaaccaagactagatgttaattgccagagaaatagataagaatgcctatgacagcagtacattaatatgatatcaaagcttggaaattttattggtaatgaataattcagtacttaaaatatttagaagctatagaattaaaattaattaatgttgttcactgtgtgaataaagttgattgagattttacatttaattttgtaaacccagtgttatcttttccagctcagaaaacaccacatacaagctactactttctgttttgatcccttatttttctttcttatgctttatcactgaaaactctccttgagcaggccatgcactgtaaatatttctcctggttgcaaaaccttctcatacaaatgcagtagactgtgtaatgagctcttctttcacaaaattaaaaaaacctgaaagccctgatttgcgattctatacaaatgagatttagatctaacaattttaaattattgcttcactcttagctgttcaattctatctcttatttgggaaaccgaaataataaaaccattgctgattccacaattaggttgtaaaagtcaccgtagccatcagccatgaagcaaaagtgccaagatcaaaactacaaagcaaagaggctgagataaaaatgctgcagcattagtttatagcattataagcagcaataagaattccttgattgcttaacaaagactcaaaaggcatttactccattaccttacaactcaaagaggtattcctggaccagcagtattggcatttttttgaagtttgtaggaaatgcagaattttggtgcctccacggacctaatgcagcagaacttgcagtttagtaagatctccaggagatttgtatgcgcattaaagtctaggaagcaccgctatggtatacatctgatgtgtgcccatgcattttttaaaagtatgaagtaatagttgtaagtattggacactcttgaaggaacaaataagagccatggtctttactctctaaatacctccctgacatctatgttttaggcaaaatttttttcccatttcagtagtcactgatgcttgcacgatgcagtttattccaaaacaatggtgattctcatgtaatagttcatgttgccttaataatttacgttgcctcaagttctctgcccaggccccaatatacaccgagggctgtactcctcccctaacgcctgctctcatacagtggcatagagcccagttttatgctcttggtcacatcatggagattgcacaccacaggctttaacttctgccgtactctcactgcctctaaccctccatatgcctaagttctacgattctttaaattccaaattgacccagaagtctcctccgctcatccttttcactgagatcatccctcttctggcctaccatttgttgatcaccttgctttttttttatcctactgtatgtagtataacaaattatcacttgcaactgtgtcttattttttcaactagattatgtactgcctaagacctagaaaattgtgcttatttatttgaatctctaggaggatcagtaatgggtattaatactaatgactccatggtgatgatgagcctgaacttcctcccttcctttctttctacctctctcctttcctcccttcttttcttcctccattccttcctctcttcctccctccgcttcttccccacttcccttattcatagattcatgcgttcactcagcaaatgcttactgaaaccttccatgcatcagacattgtactaaacaataggaaactatcatgaataagacacaatatctgacctcaaagaatttatgatataaaagtaatggcataaaccgtgattacttttgcaccaacctaatatatagacacagtttgttatgactggtgtctctattactaagcaatgactgtcacatgcaacgctgatctgaacaggtggtaaagagtgagatgtaagcaatggagcaaagccaactagttacaaggaaatatcacatgtttactagagcacatctcatgggcattcaagagagtatggccaggacagcttgtgaatagttcagtaactgtgcatagttttatattcattgtgaggcaccgtgtcaccggtttgctgatttacagagtattttaattgctaactgtatgctaccaaaatttccagtattcgaaaataattttgcttgaatgtagaaaaagaaaaaagccaagaaatgtatgtgaaacgagagtctaagggagctttacctcagtctcagaaaacatgcattccttccttcatttaggaagcatgtactggggtctactgtcagcttgctattgtgtcaaggagtaggagaatacaaaaatattagagaatatgaatcacatctattaggagagttttctacatacgcacattattctgtcagtgacataaggatttgagtcattcagatttaaatacggtaggtacctcaagttctcagatattatttcattttctaaggttcgtatttagttaatatgttattttaatggccttacaaattctagattatcttttttaaaaagttaaatagaacgtaattgccatttttatttaatggtaaaaagcatttttgtttttgtgtgtacttggttgtaatattctccttttcaattgagctatttttctgatactttactcttaaaatttcattcaggaaaaaagtaaacaatatttaagcttgacaatcataaaaatgctctggtgactatagattattttaaaatttattactgtagcttagggatatcttgatgggatgctcctgaaagcaattaattctcagttttttgtggcttctaatgcaaaatacattgacgcagacagaatttgaaatgaattttcttctaatatagcaattaattttatttaaatatctctagagtttttttttaatactgtgactaacctatgtttgttctttttcacctctcgtatccacgatcactaagaaacccaaatactttgttcatgtttaaattttacaacatttcatagactattaaacatggaacatccttgtggggacaagaaatcgaatttgctcttgaaaaggtttccaactaattgatttgtaggacattataacatcctctagctgacaagcttacaaaaataaaaactggagctaaccgagagggtgcttttttccctgacacataaaaggtgtctttctgtcttgtatcctttggatatgggcatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacag

SEQ ID NO: 278 - DNA, 45 bp, Homo sapiens
  source 1..45: mol_type = genomic DNA
ttaacttcttaaagatcaggttctgaagggtgatggaaattactt

SEQ ID NO: 279 - DNA, 60 bp, Homo sapiens
  source 1..60: mol_type = genomic DNA
tttttgttttgcctttttggtatcttacaggaactccaggatggcattgggcagcggcaa

SEQ ID NO: 280 - DNA, 176 bp, Homo sapiens
  source 1..176: mol_type = genomic DNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 281 - DNA, 50 bp, Homo sapiens
  source 1..50: mol_type = genomic DNA
gaactccaggatggcattgggcagcggcaaactgttgtcagaacattgaa

SEQ ID NO: 282-322 - skipped (41 entries)

SEQ ID NO: 323 - DNA, 250 bp, Homo sapiens
  source 1..250: mol_type = genomic DNA
tgacaagcttacaaaaataaaaactggagctaaccgagagggtgcttttttccctgacacataaaaggtgtctttctgtcttgtatcctttggatatgggcatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacag

SEQ ID NO: 324 - protein, 7 aa, synthetic construct
  REGION 1..7: note = Synthetic
ASSLNIA

SEQ ID NO: 325 - protein, 12 aa, synthetic construct
  REGION 1..12: note = Synthetic
SKTFNTHPQSTP

SEQ ID NO: 326 - protein, 12 aa, synthetic construct
  REGION 1..12: note = Synthetic
TARGEHKEEELI

SEQ ID NO: 327 - protein, 9 aa, synthetic construct
  REGION 1..9: note = Synthetic
CQAQGQLVC

SEQ ID NO: 328 - protein, 9 aa, synthetic construct
  REGION 1..9: note = Synthetic
CSERSMNFC

SEQ ID NO: 329 - protein, 9 aa, synthetic construct
  REGION 1..9: note = Synthetic
CPKTRRVPC

SEQ ID NO: 330 - protein, 20 aa, synthetic construct
  REGION 1..20: note = Synthetic
WLSEAGPVVTVRALRGTGSW

SEQ ID NO: 331 - protein, 9 aa, synthetic construct
  REGION 1..9: note = Synthetic
CMQHSMRVC

SEQ ID NO: 332 - protein, 7 aa, synthetic construct
  REGION 1..7: note = Synthetic
DDTRHWG

SEQ ID NO: 333 - protein, 10 aa, synthetic construct
  REGION 1..10: note = Synthetic
GYTFTNYWMH

SEQ ID NO: 334 - protein, 17 aa, synthetic construct
  REGION 1..17: note = Synthetic
EINPINGRSNYGERFKT

SEQ ID NO: 335 - protein, 7 aa, synthetic construct
  REGION 1..7: note = Synthetic
GTRAMHY

SEQ ID NO: 336 - protein, 11 aa, synthetic construct
  REGION 1..11: note = Synthetic
RTSENIYNNLA

SEQ ID NO: 337 - protein, 7 aa, synthetic construct
  REGION 1..7: note = Synthetic
AATNLAD

SEQ ID NO: 338 - protein, 10 aa, synthetic construct
  REGION 1..10: note = Synthetic
QHFWGTPLTF

SEQ ID NO: 339 - protein, 116 aa, synthetic construct
  REGION 1..116: note = Synthetic
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 340 - protein, 107 aa, synthetic construct
  REGION 1..107: note = Synthetic
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 341 - protein, 9 aa, synthetic construct
  REGION 1..9: note = Synthetic
YTFTNYWMH

SEQ ID NO: 342 - protein, 17 aa, synthetic construct
  REGION 1..17: note = Synthetic
EINPINGRSNYAQKFQG

SEQ ID NO: 343 - protein, 17 aa, synthetic construct
  REGION 1..17: note = Synthetic
EINPINGRSNYAEKFQG

SEQ ID NO: 344 - protein, 17 aa, synthetic construct
  REGION 1..17: note = Synthetic
EINPIQGRSNYAEKFQG

SEQ ID NO: 345 - protein, 7 aa, synthetic construct
  REGION 1..7: note = Synthetic
AATNLAE

SEQ ID NO: 346 - protein, 11 aa, synthetic construct
  REGION 1..11: note = Synthetic
RTSENIYSNLA

SEQ ID NO: 347 - protein, 7 aa, synthetic construct
  REGION 1..7: note = Synthetic
AGTNLAD

SEQ ID NO: 348 - protein, 116 aa, synthetic construct
  REGION 1..116: note = Synthetic
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 349 - protein, 116 aa, synthetic construct
  REGION 1..116: note = Synthetic
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 350 - protein, 116 aa, synthetic construct
  REGION 1..116: note = Synthetic
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 351 - protein, 116 aa, synthetic construct
  REGION 1..116: note = Synthetic
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 352 - protein, 107 aa, synthetic construct
  REGION 1..107: note = Synthetic
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 353 - protein, 107 aa, synthetic construct
  REGION 1..107: note = Synthetic
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 354 - protein, 107 aa, synthetic construct
  REGION 1..107: note = Synthetic
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 355 - protein, 107 aa, synthetic construct
  REGION 1..107: note = Synthetic
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK